(19) United States
(12) Patent Application Publication
(10) Pub. No.: US 2024/0101668 A1
(43) Pub. Date: Mar. 28, 2024

(54) POLYPEPTIDES AND THEIR USE IN TREATMENT OF DISEASE

(71) Applicant: WUGEN, Inc., St. Louis, MO (US)

(72) Inventors: Ayman KABAKIBI, San Diego, CA (US); Alexander S. HAMIL, San Diego, CA (US)

(21) Appl. No.: 18/447,138

(22) Filed: Aug. 9, 2023

Related U.S. Application Data
(63) Continuation of application No. PCT/US2022/015980, filed on Feb. 10, 2022.
(60) Provisional application No. 63/148,012, filed on Feb. 10, 2021.

(51) Int. Cl. C07K 16/28; A61K 39/00; A61P 37/04; C12N 15/86

(52) U.S. Cl. CPC C07K 16/2803; A61K 39/4611; A61K 39/4631; A61P 37/04; C07K 16/2851; C07K 16/2863; C12N 15/86; A61K 2239/11; A61K 2239/21; C07K 2317/24; C07K 2317/565; C07K 2317/622; C12N 2740/15043

(57) ABSTRACT

Disclosed herein are polypeptides, such as monoclonal antibodies (mAbs) and functional fragments thereof, synthetic antigen-binding proteins such as single-chain variable fragments (scFvs), and chimeric antigen receptors (CARs), that can specifically recognize tumor-associated antigens (TAAs) on cancer cells, for example those that express CD33, FLT3, and CLL-1, useful in the treatment of diseases such as cancer.

### CROSS REFERENCE TO RELATED APPLICATIONS

This application is a bypass continuation of International Application No. PCT/US2022/015980, filed Feb. 10, 2022, which claims priority to, and the benefit of, U.S. Provisional Patent Application No. 63/148,012, filed Feb. 10, 2021, the entireties of each are incorporated herein by reference as if written in their entireties.

### INCORPORATION OF SEQUENCE LISTING

The sequence listing that is contained in the file named “WGN0011-201BC1-US,” which is 3.57 megabytes as measured in Microsoft Windows operating system and was created on Aug. 8, 2023, is filed electronically herewith and incorporated herein by reference.

Targeted immunotherapies are based on the recognition of antigens, defined structures on diseased cells or pathogens, by immune receptors that are either soluble, i.e., antibodies, or present on the surface of immune cells, such as chimeric antigen receptors (CARs) in CAR-bearing immune effector cells such as CAR-T cells. Recognition and binding of the antigen by the immune receptor usually triggers effector functions that eventually lead to the destruction of the respective pathogen or cell. Soluble immune receptors include natural or synthetic antibodies, antibody derived molecules and other structures, which upon binding to an antigen trigger the complement system or recruit and in most cases activate effector cells. Alternatively, antigen-targeting cells can be generated through the genetic insertion of engineered immune receptors, such as transgenic T-cell receptors (TCRs) or CARs into T cells or other immune effector cells including natural killer (NK) cells. Commonly, CARs comprise a single chain fragment variable (scFv) derived from an antibody specific for a certain target antigen coupled via hinge and transmembrane regions to cytoplasmic domains of T-cell signaling molecules. The CAR-mediated adoptive immunotherapy allows CAR-grafted cells to directly recognize the desired antigen on target cells in a non-HLA-restricted manner.

One common application of these immunotherapies, though not the only application, is the treatment of cancer. Cancer is a broad group of diseases involving deregulated cell growth. In cancer, cells divide and grow uncontrollably, forming malignant tumors, and invading nearby parts of the body. The cancer may also spread to more distant parts of the body through the lymphatic system or bloodstream. There are over 200 different known cancers that affect humans. Whereas good treatment options are available for many cancer types, others still represent unmet medical need.

Amongst these are hematologic cancers. Cancers of the hematopoietic system can be roughly divided into different subtypes. Leukemias generally affect the primary lymphatic organs, which are the bone marrow as well as the thymus, and arise from hematopoietic progenitor populations, such as, for example, acute myeloid leukemia (AML). Lymphomas on the other hand are usually derived from mature lymphocytes and originate from secondary lymphatic organs.

The current first line treatment for most hematopoietic cancers involves the administration of chemotherapeutic agents (either broad-spectrum or targeted therapies), radiation therapy or a combination of both. In many cases such therapies are combined with or followed by hematopoietic stem cell transfer (HSCT), where the graft versus leukemia (GvL) effect mediated by donor-derived lymphocytes, especially T cells, can lead to the eradication of cancer cells that survived pre-conditioning chemo- or radiotherapies and result in complete remission (CR). Depending on the type of hematological malignancy, the patients' condition, and the availability of hematopoietic stem cell grafts, various versions of HSCT are regularly performed in the clinics. The desired GvL effect is, at present, only achieved in allogeneic HSCT, which at the same time is often accompanied by the occurrence of graft versus host disease (GvHD), a serious and sometimes fatal complication. Moreover, in all cases, persisting cancer stem cells often lead to disease relapse.

In recent years there has been strong progress in the development of targeted immunotherapies, such as CAR-T cells, for the treatment of cancer. However, most broadly target antigens which are expressed on malignant as well as healthy cells, and do so using polypeptides which target antigens in a polymorphically nonselective manner. Additionally, relapse of hematologic cancer in patients transplanted with HSCT remains a problem to be solved. Therefore, there is a need for the development of novel therapies for the treatment of diseases, such as cancer, that enable the utilization of alternative target molecules, and reduce or avoid the side-effects often associated with current targeted immunotherapies in general, and CAR T cell therapies in particular.

### SUMMARY

Provided herein are polymorphically selective polypeptides, including single-chain variable fragments, monoclonal antibodies and antigen-binding fragments thereof, antibody-drug conjugates, and chimeric antigen receptors and engineered immune effector cells comprising them, useful in the treatment of diseases such as cancer, and in some embodiments, in combination with polymorphically mismatched hematopoietic cell transplant in a manner that permits selective killing of the patient's diseased cells while sparing transplanted hematopoietic cells.

### BRIEF DESCRIPTION OF THE SEQUENCES

SEQ ID NOs:1-200: sequences of CDRs and VH and VL chains of polymorphically selective anti-CD33 polypeptides 1-25.

SEQ ID NOs:201-336: sequences of CDRs and VH and VL chains of polymorphically selective anti-CD33 polypeptides 26-42.

SEQ ID NOs:337-528: sequences of CDRs and VH and VL chains of polymorphically selective anti-CLL-1 polypeptides 43-66.

SEQ ID NOs:529-704: sequences of CDRs and VH and VL chains of polymorphically selective anti-CLL-1 polypeptides 67-88.

SEQ ID NOs:705-1144 and 1979-2002: sequences of CDRs and VH and VL chains of polymorphically nonselective anti-CD33 polypeptides 89-143 and 191-193.

SEQ ID NOs:1145-1520 and 2003-2058: sequences of CDRs and VH and VL chains of polymorphically nonselective anti-CLL-1 polypeptides 144-190 and 194-200.

SEQ ID NOs: 1521-1538: amino acid sequences of selected CAR components.

SEQ ID NOs:1539-1598: sequences of exemplary CARs which may be made using the polypeptides disclosed herein.

SEQ ID NOs:1599-1626: Human antibody Fc components which may be combined with polypeptides disclosed herein to form diagnostic or therapeutic antibodies.

SEQ ID NOs: 1627-1802 sequences of exemplary anti-CD33 and anti-CLL-1 IgG1 antibodies comprising Polypeptides 1-88.

SEQ ID NOs: 1803-1978: sequences of exemplary anti-CD33 and anti-CLL-1 IgG4 antibodies comprising Polypeptides 1-88.

SEQ ID NOs:2059-2810: sequences of CDRs and VH and VL chains of polymorphically nonselective anti-FLT3 polypeptides 201-294.

### BRIEF DESCRIPTION OF THE DRAWINGS

FIG. 1 shows the CD33 extracellular domain (ECD) with amino acid (AA) 69 in the left panel, and FLT3 ECD AA267 in the right panel, each in a relatively solvent-accessible position.

FIG. 2 shows control (C) parental Jurkat cells, Jurkat cells expressing CD33-R69 (R69), and Jurkat cells expressing CD33-G69 (G69), and treated with:
A: PD-L1 scFv as a negative control, yielding a mean fold intensity change in R69 and G69 over parental cells of 0.73 and 0.66, respectively;
B: CD33 nonselective scFv as a positive control, yielding a MFI change in R69 and G69 over parental cells of 78.9 and 74.6, respectively;
C: CD33-R69 selective scFv, yielding a MFI change in R69 and G69 over parental cells of 20.3 and 0.58, respectively; and
D: CD33-G69 selective scFv, yielding a MFI change in R69 and G69 over parental cells of 0.59 and 45.9, respectively.

FIG. 3 shows the fold selectivity of polypeptides 1-42 against huCD33-R69 or huCD33-G69 stably expressed in Jurkat cells.

FIG. 4 shows the results of an vitro cytotoxicity assay wherein a culture of CD33^{GLY69} cell targets are treated with CART33^{ARG69}, CART33^{GLY69} or CART33. CART33^{GLY69} and CART33, but not CART33^{ARG69}, effectively kill CD33^{GLY69}-expressing cells.

FIG. 5 shows the results of an vitro cytotoxicity assay wherein a culture of CD33^{ARG69} cell targets are treated with CART33^{ARG69}, CART33^{GLY69} or CART33. CART33^{ARG69} and CART33, but not CART33^{GLY69}, effectively kill CD33^{ARG69}-expressing cells.

### DETAILED DESCRIPTION

Provided herein are polymorphically selective polypeptides, including single-chain variable fragments, monoclonal antibodies and antigen-binding fragments thereof, antibody-drug conjugates, and chimeric antigen receptors and engineered immune effector cells comprising them, useful in the treatment of diseases such as cancer, and in some embodiments, in combination with polymorphically mismatched hematopoietic cell transplant in a manner that permits selective killing of the patient's diseased cells while sparing transplanted hematopoietic cells.

### Embodiments

Accordingly, although other embodiments may be found throughout the disclosure, provided herein are the following embodiments:

Embodiment 1. A polypeptide which selectively binds a first polymorphic variant of a human cancer cell antigen over a second polymorphic variant of the human cancer cell antigen; or selectively binds the second polymorphic variant of the antigen over the first polymorphic variant of the antigen.

Embodiment 2. The polypeptide of embodiment 1, wherein the antigen is chosen from CD33, CLL-1, and FLT3.

Embodiment 3. The polypeptide of embodiment 2, wherein the antigen is CD33.

Embodiment 4. A polypeptide which selectively binds a first polymorphic variant of CD33 over a second polymorphic variant of CD33; or selectively binds the second polymorphic variant of CD33 over the first polymorphic variant of CD33; wherein the binding is at least 2-fold selective.

Embodiment 5. The polypeptide of embodiment 4, wherein the binding is at least 10-fold selective.

Embodiment 6. The polypeptide of embodiment 5, wherein the binding is at least 30-fold selective.

Embodiment 7. The polypeptide of any of embodiments 3-6, wherein the first polymorphic variant of CD33 is R69 and the second polymorphic variant of CD33 is G69; or the first polymorphic variant of CD33 is G69 and the second polymorphic variant of CD33 is R69.

Embodiment 8. The polypeptide of embodiment 7, comprising six complementarity-determining regions (CDRs).

Embodiment 9. The polypeptide of embodiment 8, comprising:
three heavy chain variable (V_{H}) domain CDRs: HCDR1, HCDR2, and HCDR3; and
three light chain variable (V_{L}) domain CDRs: LCDR1, LCDR2, and LCDR3.

Embodiment 10. The polypeptide of any of embodiments 7-9, wherein:
HCDR1 comprises an amino acid sequence with at least 95% sequence identity to an amino acid sequence chosen from SEQ ID NOs:1-25 and 201-217,
HCDR2 comprises an amino acid sequence with at least 95% sequence identity to an amino acid sequence chosen from SEQ ID NOs:26-50 and 218-234,
HCDR3 comprises an amino acid sequence with at least 95% sequence identity to an amino acid sequence chosen from SEQ ID NOs:51-75 and 235-251.

Embodiment 11. The polypeptide of any of embodiments 7-9, wherein:
HCDR1 comprises an amino acid sequence with at least 95% sequence identity to an amino acid sequence chosen from SEQ ID NOs:1-25,
HCDR2 comprises an amino acid sequence with at least 95% sequence identity to an amino acid sequence chosen from SEQ ID NOs:26-50, and
HCDR3 comprises an amino acid sequence with at least 95% sequence identity to an amino acid sequence chosen from SEQ ID NOs:51-75.

Embodiment 12. The polypeptide of any of embodiments 7-9, wherein:
HCDR1 comprises an amino acid sequence with at least 95% sequence identity to an amino acid sequence chosen from SEQ ID NOs: 201-217,
HCDR2 comprises an amino acid sequence with at least 95% sequence identity to an amino acid sequence chosen from SEQ ID NOs: 218-234, and
HCDR3 comprises an amino acid sequence with at least 95% sequence identity to an amino acid sequence chosen from SEQ ID NOs: 235-251.

Embodiment 13. The polypeptide of any of embodiments 10-12, wherein the HCDR1, HCDR2, and HCDR3 have at least 97%, 98% or 99% sequence identity to one of the recited amino acid sequences.

Embodiment 14. The polypeptide of any of embodiments 10-12, wherein the HCDR1, HCDR2, and HCDR3 have the recited amino acid sequences.

Embodiment 15. The polypeptide of any of embodiments any of embodiments 7-9 and 8-14, wherein:
LCDR1 comprises an amino acid sequence with at least 95% sequence identity to an amino acid sequence chosen from SEQ ID NOs:76-100 and 252-268,
LCDR2 comprises an amino acid sequence with at least 95% sequence identity to an amino acid sequence chosen from SEQ ID NOs:101-125 and 269-285, and
LCDR3 comprises an amino acid sequence with at least 95% sequence identity to an amino acid sequence chosen from SEQ ID NOs:126-150 and 286-302.

Embodiment 16. The polypeptide of any of embodiments any of embodiments 7-9 and 8-14, wherein:
LCDR1 comprises an amino acid sequence with at least 95% sequence identity to an amino acid sequence chosen from SEQ ID NOs: 76-100,
LCDR2 comprises an amino acid sequence with at least 95% sequence identity to an amino acid sequence chosen from SEQ ID NOs:101-125, and
LCDR3 comprises an amino acid sequence with at least 95% sequence identity to an amino acid sequence chosen from SEQ ID NOs:126-150.

Embodiment 17. The polypeptide of any of embodiments any of embodiments 7-9 and 8-14, wherein:
LCDR1 comprises an amino acid sequence with at least 95% sequence identity to an amino acid sequence chosen from SEQ ID NOs: 252-268,
LCDR2 comprises an amino acid sequence with at least 95% sequence identity to an amino acid sequence chosen from SEQ ID NOs: 269-285, and
LCDR3 comprises an amino acid sequence with at least 95% sequence identity to an amino acid sequence chosen from SEQ ID NOs: 286-302.

Embodiment 18. The polypeptide of any of embodiments 15-17, wherein the LCDR1, LCDR2, and LCDR3 have at least 97%, 98% or 99% sequence identity to one of the recited amino acid sequences.

Embodiment 19. The polypeptide of any of embodiments 15-17, wherein the LCDR1, LCDR2, and LCDR3 have the recited amino acid sequences.

Embodiment 20. The polypeptide of embodiment 7, comprising a VH domain having an amino acid sequence exhibiting at least 95% sequence identity to a sequence chosen from any of SEQ ID NOs 151-175 and 303-319.

Embodiment 21. The polypeptide of embodiment 7, comprising a V_{H} domain having an amino acid sequence exhibiting at least 95% sequence identity to a sequence chosen from any of SEQ ID NOs 151-175.

Embodiment 22. The polypeptide of embodiment 7, comprising a V_{H} domain having an amino acid sequence exhibiting at least 95% sequence identity to a sequence chosen from any of SEQ ID NOs 303-319.

Embodiment 23. The polypeptide of any of embodiments 20-22, wherein the V_{H} domain has at least 97%, 98% or 99% sequence identity to one of the recited amino acid sequences.

Embodiment 24. The polypeptide of any of embodiments 20-22, wherein the V_{H} domain has one of the recited amino acid sequences.

Embodiment 25. The polypeptide of any of embodiments 7 and 20-24, comprising a V_{L} domain having an amino acid sequence exhibiting at least 95% sequence identity to a sequence chosen from any of SEQ ID NOs 176-200 and 320-336.

Embodiment 26. The polypeptide of any of embodiments 7 and 20-24, comprising a V_{L} domain having an amino acid sequence exhibiting at least 95% sequence identity to a sequence chosen from any of SEQ ID NOs 176-200.

Embodiment 27. The polypeptide of any of embodiments 7 and 20-24, comprising a V_{L} domain having an amino acid sequence exhibiting at least 95% sequence identity to a sequence chosen from any of SEQ ID NOs 320-336.

Embodiment 28. The polypeptide of any of embodiments 25-27, wherein the V_{L} domain has at least 97%, 98% or 99% sequence identity to one of the recited amino acid sequences.

Embodiment 29. The polypeptide of any of embodiments 25-27, wherein the V_{L} domain has one of the recited amino acid sequences.

Embodiment 30. The polypeptide of any of embodiments 20-29, comprising a combination of a V_{H} domain and a V_{L} domain, wherein the combination is chosen from those recited in Polypeptide No.s 1-42.

Embodiment 31. The polypeptide of any of embodiments 20-29, comprising a combination of a V_{H} domain and a V_{L} domain, wherein the combination is chosen from those recited in Polypeptide No.s 1-25.

Embodiment 32. The polypeptide of any of embodiments 20-29, comprising a combination of a V_{H} domain and a V_{L} domain, wherein the combination is chosen from those recited in Polypeptide No.s 26-42.

Embodiment 33. The polypeptide of any of embodiments 30-33, wherein the V_{H} and V_{L} domains have at least 97%, 98% or 99% sequence identity to one of the recited amino acid sequence pairs.

Embodiment 34. The polypeptide of embodiment 2, wherein the antigen is FLT3.

Embodiment 35. A polypeptide which selectively binds a first polymorphic variant of FLT3 over a second polymorphic variant of FLT3; or selectively binds the second polymorphic variant of FLT3 over the first polymorphic variant; wherein the binding is at least 2-fold selective.

Embodiment 36. The polypeptide of embodiment 35, wherein the binding is at least 10-fold selective.

Embodiment 37. The polypeptide of embodiment 36, wherein the binding is at least 30-fold selective.

Embodiment 38. The polypeptide of any of embodiments 34-37, wherein the first polymorphic variant of FLT3 is T227 and the second polymorphic variant of FLT3 is M227; or first polymorphic variant of FLT3 is M227 and the second polymorphic variant of FLT3 is T227.

Embodiment 39. The polypeptide of embodiment 2, wherein the antigen is CLL-1.

Embodiment 40. A polypeptide which selectively binds a first polymorphic variant of CLL-1 over a second polymorphic variant of CLL-1; or selectively binds the second polymorphic variant of CLL-1 over the first polymorphic variant; wherein the binding is at least 2-fold selective.

Embodiment 41. The polypeptide of embodiment 40, wherein the binding is at least 10-fold selective.

Embodiment 42. The polypeptide of embodiment 40, wherein the binding is at least 30-fold selective.

Embodiment 43. The polypeptide of any of embodiments 39-42, wherein the first polymorphic variant of CLL-1 is K224 and the second polymorphic variant of CLL-1 is Q244; or first polymorphic variant of CLL-1 is Q224 and the second polymorphic variant of CLL-1 is K244.

Embodiment 44 The polypeptide of claim 43, comprising six complementarity-determining regions (CDRs).

Embodiment 45. The polypeptide of Embodiment 44, comprising: three heavy chain variable (V_{H}) domain CDRs: HCDR1, HCDR2, and HCDR3; and three light chain variable (V_{L}) domain CDRs: LCDR1, LCDR2, and LCDR3.

Embodiment 46. The polypeptide of any of Embodiments 43-45, wherein:
HCDR1 comprises an amino acid sequence with at least 95% sequence identity to an amino acid sequence chosen from SEQ ID NOs:337-360- and 529-550,
HCDR2 comprises an amino acid sequence with at least 95% sequence identity to an amino acid sequence chosen from SEQ ID NOs:361-384 and 551-572, and
HCDR3 comprises an amino acid sequence with at least 95% sequence identity to an amino acid sequence chosen from SEQ ID NOs:385-408 and 573-594.

Embodiment 47. The polypeptide of any of Embodiments 43-45, wherein:
HCDR1 comprises an amino acid sequence with at least 95% sequence identity to an amino acid sequence chosen from SEQ ID NOs: 337-360,
HCDR2 comprises an amino acid sequence with at least 95% sequence identity to an amino acid sequence chosen from SEQ ID NOs: 361-384, and
HCDR3 comprises an amino acid sequence with at least 95% sequence identity to an amino acid sequence chosen from SEQ ID NOs: 385-408.

Embodiment 48. The polypeptide of any of Embodiments 43-45, wherein:
HCDR1 comprises an amino acid sequence with at least 95% sequence identity to an amino acid sequence chosen from SEQ ID NOs: 529-550,
HCDR2 comprises an amino acid sequence with at least 95% sequence identity to an amino acid sequence chosen from SEQ ID NOs: 551-572, and
HCDR3 comprises an amino acid sequence with at least 95% sequence identity to an amino acid sequence chosen from SEQ ID NOs: 573-594.

Embodiment 49. The polypeptide of any of Embodiments 46-48, wherein the HCDR1, HCDR2, and HCDR3 have at least 97%, 98% or 99% sequence identity to one of the recited amino acid sequences.

Embodiment 50. The polypeptide of any of Embodiments 46-48, wherein the HCDR1, HCDR2, and HCDR3 have the recited amino acid sequences.

Embodiment 51. The polypeptide of any of Embodiments 43-50, wherein:
LCDR1 comprises an amino acid sequence with at least 95% sequence identity to an amino acid sequence chosen from SEQ ID NOs:409-432 and 595-616,
LCDR2 comprises an amino acid sequence with at least 95% sequence identity to an amino acid sequence chosen from SEQ ID NOs:433-456 and 617-638, and
LCDR3 comprises an amino acid sequence with at least 95% sequence identity to an amino acid sequence chosen from SEQ ID NOs:457-480 and 639-660.

Embodiment 52. The polypeptide of any of Embodiments 43-50, wherein:
LCDR1 comprises an amino acid sequence with at least 95% sequence identity to an amino acid sequence chosen from SEQ ID NOs: 409-432,
LCDR2 comprises an amino acid sequence with at least 95% sequence identity to an amino acid sequence chosen from SEQ ID NOs: 433-456, and
LCDR3 comprises an amino acid sequence with at least 95% sequence identity to an amino acid sequence chosen from SEQ ID NOs: 457-480.

Embodiment 53. The polypeptide of any of Embodiments 43-50, wherein:
LCDR1 comprises an amino acid sequence with at least 95% sequence identity to an amino acid sequence chosen from SEQ ID NOs: 595-616,
LCDR2 comprises an amino acid sequence with at least 95% sequence identity to an amino acid sequence chosen from SEQ ID NOs: 617-638, and
LCDR3 comprises an amino acid sequence with at least 95% sequence identity to an amino acid sequence chosen from SEQ ID NOs: 639-660.

Embodiment 54. The polypeptide of any of Embodiments 51-53, wherein the LCDR1, LCDR2, and LCDR3 have at least 97%, 98% or 99% sequence identity to one of the recited amino acid sequences.

Embodiment 55. The polypeptide of any of Embodiments 51-53, wherein the LCDR1, LCDR2, and LCDR3 have the recited amino acid sequences.

Embodiment 56. The polypeptide of Embodiment 44, comprising a V_{H} domain having an amino acid sequence exhibiting at least 95% sequence identity to a sequence chosen from any of SEQ ID NOs 151-175 and 303-319.

Embodiment 57. The polypeptide of Embodiment 44, comprising a V_{H} domain having an amino acid sequence exhibiting at least 95% sequence identity to a sequence chosen from any of SEQ ID NOs 151-175.

Embodiment 58. The polypeptide of Embodiment 44, comprising a V_{H} domain having an amino acid sequence exhibiting at least 95% sequence identity to a sequence chosen from any of SEQ ID NOs 303-319.

Embodiment 59. The polypeptide of any of Embodiments 56-58, wherein the V_{H} domain has at least 97%, 98% or 99% sequence identity to one of the recited amino acid sequences.

Embodiment 60. The polypeptide of any of Embodiments 56-58, wherein the V_{H} domain has one of the recited amino acid sequences.

Embodiment 61. The polypeptide of any of Embodiments 44 and 56-60, comprising a V_{L} domain having an amino acid sequence exhibiting at least 95% sequence identity to a sequence chosen from any of SEQ ID NOs 176-200 and 320-336.

Embodiment 62. The polypeptide of any of Embodiments 44 and 56-60, comprising a V_{L} domain having an amino acid sequence exhibiting at least 95% sequence identity to a sequence chosen from any of SEQ ID NOs 176-200.

Embodiment 63. The polypeptide of any of Embodiments 44 and 56-60, comprising a V_{L} domain having an amino acid sequence exhibiting at least 95% sequence identity to a sequence chosen from any of SEQ ID NOs 320-336.

Embodiment 64. The polypeptide of any of Embodiments 61-63, wherein the V_{L} domain has at least 97%, 98% or 99% sequence identity to one of the recited amino acid sequences.

Embodiment 65. The polypeptide of any of Embodiments 61-63, wherein the V_{L} domain has one of the recited amino acid sequences.

Embodiment 66. The polypeptide of any of Embodiments 56-65, comprising a combination of a V_{H} domain and a V_{L} domain, wherein the combination is chosen from those recited in Polypeptide No.s 43-88.

Embodiment 67. The polypeptide of any of Embodiments 56-65, comprising a combination of a V_{H} domain and a V_{L} domain, wherein the combination is chosen from those recited in Polypeptide No.s 43-66.

Embodiment 68. The polypeptide of any of Embodiments 56-65, comprising a combination of a V_{H} domain and a V_{L} domain, wherein the combination is chosen from those recited in Polypeptide No.s 67-88.

Embodiment 69. The polypeptide of any of Embodiments 66-68, wherein the V_{H} and V_{L} domains have at least 97%, 98% or 99% sequence identity to one of the recited amino acid sequence pairs.

Embodiment 70. A single-chain variable fragment (scFv) comprising the polypeptide of any of Embodiments 1-69.

Embodiment 71. A monoclonal antibody (mAb), or an antigen-binding fragment thereof, comprising the polypeptide of any of Embodiments 1-69.

Embodiment 72. The mAb, or antigen-binding fragment thereof, of Embodiment 71, wherein the mAb is of the IgG, IgM, or IgA isotype.

Embodiment 73. The mAb, or antigen-binding fragment thereof, of Embodiment 72, wherein the mAb is of the IgG1 isotype.

Embodiment 74. The mAb, or antigen-binding fragment thereof, of Embodiment 72, wherein the mAb is of the IgG3 isotype.

Embodiment 75. The mAb, or antigen-binding fragment thereof, of Embodiment 72, wherein the mAb is of the IgG4 isotype.

Embodiment 76. The mAb, or antigen-binding fragment thereof, of Embodiment 72, wherein the mAb is human or humanized.

Embodiment 77. The mAb, or antigen-binding fragment thereof, of any of Embodiments 71-76, wherein the mAb comprises a sequence chosen from SEQ ID NOs: 1201-1368.

Embodiment 78. An antibody-drug conjugate (ADC) comprising the mAb, or antigen-binding fragment thereof, of any of Embodiments 71-77.

Embodiment 79. The ADC of Embodiment 52, having Formula I:

Ab-(L-D)ₚ (I)

wherein:
Ab is an antibody comprising the polypeptide of any of Embodiments 1-43, or the antibody of any of Embodiments 45-51, or an antigen-binding fragment of either of the foregoing;
L is a linker;
D is a drug; and
p is about 1 to about 20.

Embodiment 80. The ADC of Embodiment 79, wherein D is chosen from saporin, MMAE, MMAF, DM1, and DM4.

Embodiment 81. A chimeric antigen receptor (CAR) comprising an extracellular ligand binding domain comprising a polypeptide of any one of Embodiments 1-69.

Embodiment 82. The CAR of Embodiment 81, additionally comprising:
a hinge domain;
a transmembrane domain;
optionally, one or more co-stimulatory domains; and
a cytoplasmic signaling domain.

Embodiment 83. The CAR of Embodiment 82, wherein the hinge domain is chosen from FcεRIIIa, CD8α, CD28 and IgG1.

Embodiment 84. The CAR of Embodiment 83, wherein the hinge domain is CD8α.

Embodiment 85 The CAR of any of Embodiments 82-84, wherein the transmembrane domain is chosen from alpha, beta or zeta chain of the T-cell receptor, CD28, CD3 epsilon, CD45, CD4, CD5, CD9, CD16, CD22, CD33, CD37, CD64, CDS0, CD86, CD134, CD137 and CD154.

Embodiment 86. The CAR of Embodiment 85, wherein the transmembrane domain is CD28.

Embodiment 87. The CAR of any of Embodiments 82-86, wherein the cytoplasmic signaling domain is chosen from CD8, CD3ζ, CD3δ, CD3γ, CD3ε, CD22, CD32, DAP10, DAP12, CD66d, CD79a, CD79b, FcγRIγ, FcγRIIIγ, FcεRIβ, FcεRIγ, FcRγ, FcRβ, and FcRε.

Embodiment 88. The CAR of Embodiment 87, wherein the cytoplasmic signaling domain is CD3ζ.

Embodiment 89. The CAR of any of Embodiments 82-88, wherein one co-stimulatory domain is chosen from 4-1BB, CD28, and ICOS.

Embodiment 90. The CAR of Embodiment 89, wherein the costimulatory domain is CD28.

Embodiment 91. The CAR of Embodiment 89, wherein the costimulatory domain is 4-1BB.

Embodiment 92. The CAR of Embodiment 89, comprising two or more costimulatory domains.

Embodiment 93. The CAR of Embodiment 89, wherein two of the costimulatory domains are CD28 and 4-1BB.

Embodiment 94. The CAR of Embodiment 82, comprising a sequence chosen from SEQ ID NOs: 1539-1598.

Embodiment 95. A nucleotide sequence encoding any of the polypeptides, scFvs, mAbs, or CARs of any of Embodiments 1-94.

Embodiment 96. A vector comprising the nucleotide sequence of Embodiment 95.

Embodiment 97. The vector of Embodiment 96, wherein the vector is a lentiviral vector.

Embodiment 98. The vector of Embodiment 97, wherein the lentiviral vector comprises a VSVG domain.

Embodiment 99. An engineered immune effector cell expressing at the cell surface a CAR of any one of Embodiment 81-94.

Embodiment 100. The engineered immune effector cell of Embodiment 99, wherein the engineered immune effector cell expresses at the cell surface:
a first polymorphic variant of a human cancer cell antigen; and
a CAR that is selective for a second polymorphic over the first polymorphic variant of the antigen.

Embodiment 101. The engineered immune effector cell of Embodiment 99, wherein the cell is a primary cell.

Embodiment 102. The engineered immune effector cell of Embodiment 99, wherein the cell is derived from:
an induced pluripotent stem cell (iPSC);
cord blood;
peripheral blood; or
an immortalized cell line.

Embodiment 103. The engineered immune effector cell of Embodiment 102, wherein the immortalized cell line is NK-92.

Embodiment 104. The engineered immune cell of any of Embodiments 99-103, wherein the cell is chosen from a T cell, an natural killer (NK) cell, an invariant natural killer T (iNKT) cell, a macrophage, and a dendritic cell.

Embodiment 105. The engineered immune effector cell of Embodiment 104, wherein the cell is a T cell.

Embodiment 106. The engineered immune effector cell of Embodiment 105, wherein the T cell is chosen from an inflammatory T-lymphocyte, a cytotoxic T-lymphocyte, a regulatory T-lymphocyte, or a helper T-lymphocyte.

Embodiment 107. The engineered immune effector cell of Embodiment 105, wherein the engineered immune effector cell is deficient in a subunit of the T cell receptor complex.

Embodiment 108. The engineered immune effector cell of Embodiment 107, wherein the subunit of the T cell receptor complex is chosen from TCRα (TRAC), TCRβ, TCRδ, TCRγ, CD3ε, CD3γ, CD3δ, and CD3ζ.

Embodiment 109. The engineered immune effector cell of any of Embodiments 99-108, wherein the engineered immune effector cell is deficient in a cell surface protein that is the target of the CAR.

Embodiment 110. The engineered immune effector cell of Embodiment 104, wherein the engineered immune effector cell is an NK cell.

Embodiment 111. The engineered immune effector cell of Embodiment 110 wherein the engineered immune effector cell is a memory-like (ML) NK cell.

Embodiment 112. The engineered immune effector cell of Embodiment 111, wherein the engineered immune effector cell is a cytokine-induced memory-like (CIML) NK cell.

Embodiment 113. The engineered immune effector cell of Embodiment 104, wherein the engineered immune effector cell is an iNKT cell.

Embodiment 114. A method of treatment of a subject in need thereof, who has a first polymorphic variant of an antigen on the surface of a target cell, comprising:
a. optionally, treating the subject with one or more conditioning regimens to deplete the subject of target cells bearing the first polymorphic variant of the antigen;
b. administering to the subject either:
   a population of engineered immune effector cells that express a chimeric antigen receptor (CAR) that selectively binds the first polymorphic variant of the antigen on the surface of the target cell; or
   a monoclonal antibody (mAb), or antigen-binding fragment thereof, that selectively binds the first polymorphic variant of the antigen on the surface of the target cell; or
   an antibody-drug conjugate (ADC) comprising monoclonal antibody (mAb), or antigen-binding fragment thereof, that selectively binds the first polymorphic variant of the antigen on the surface of the target cell; and
c. administering to the subject a population of donor hematopoietic cells, a plurality of which comprise a second polymorphic variant of the antigen;
wherein the administering of the hematopoietic cells, and the administering of the CAR-expressing cells, mAb, or ADC, may be done concurrently, or sequentially in either order.

Embodiment 115. A method of immunotherapy of a human subject in need thereof, who has a first polymorphic variant of an antigen on the surface of a target cell, comprising:
a. optionally, treating the subject with one or more conditioning regimens to deplete the subject of target cells bearing the first polymorphic variant of the antigen;
b. administering to the subject a population of donor hematopoietic cells, a plurality of which comprise a second polymorphic variant of the antigen; and
c. administering to the subject:
   a population of engineered immune effector cells that express a chimeric antigen receptor (CAR) that specifically binds the first polymorphic variant of an antigen on the surface of a target cell; or
   a monoclonal antibody (mAb) or antigen-binding fragment thereof, that selectively binds the first polymorphic variant of the antigen on the surface of the target cell; or
   an antibody-drug conjugate (ADC) comprising a monoclonal antibody (mAb), or antigen-binding fragment thereof, that selectively binds the first polymorphic variant of the antigen on the surface of the target cell.

Embodiment 116. A method of treatment of a subject in need thereof, who has a first polymorphic variant of an antigen on the surface of a target cell, comprising:
a. administering to the subject:
   a population of engineered immune effector cells that express a chimeric antigen receptor (CAR) which binds the antigen on the surface of the target cell; or
   a monoclonal antibody (mAb) which binds the antigen on the surface of the target cell; or
   an antibody-drug conjugate (ADC) comprising a monoclonal antibody (mAb) which binds the antigen on the surface of the target cell; and
b. optionally, treating the subject with one or more conditioning regimens to deplete the subject of target cells bearing the first polymorphic variant of the antigen;
c. administering to the subject either:
   a population of engineered immune effector cells that express a chimeric antigen receptor (CAR) that selectively binds the first polymorphic variant of the antigen on the surface of the target cell; or
   a monoclonal antibody (mAb), or antigen-binding fragment thereof, that selectively binds the first polymorphic variant of the antigen on the surface of the target cell; or
   an antibody-drug conjugate (ADC) comprising a monoclonal antibody (mAb), or antigen-binding fragment thereof, that selectively binds the first polymorphic variant of the antigen on the surface of the target cell; and
d. administering to the subject a population of donor hematopoietic cells, a plurality of which comprise a second polymorphic variant of the antigen;
   wherein the administering of the hematopoietic cells, and the administering of the CAR-expressing cells, mAb, or ADC, may be done concurrently, or sequentially in either order.

Embodiment 117. The method of any of Embodiments 114-116, wherein the subject is a human.

Embodiment 118. The method of any of Embodiments 114-117, wherein the binding is at least 2-fold selective.

Embodiment 119. The method of Embodiment 118, wherein the binding is at east 10-fold selective.

Embodiment 120. The method of Embodiment 119, wherein the binding is at least 30-fold selective.

Embodiment 121. The method of any of Embodiments 114-120, wherein the antigen is chosen from CD33, CLL-1, and FLT3.

Embodiment 122. The method of Embodiment 121, wherein the antigen is CD33.

Embodiment 123. The method of Embodiment 122, wherein the first polymorphic variant of CD33 is R69 and the second polymorphic variant of CD33 is G69; or the first polymorphic variant of CD33 is G69 and the second polymorphic variant of CD33 is R69.

Embodiment 124. The method of Embodiment 121, wherein the antigen is FLT3.

Embodiment 125. The method of Embodiment 124, wherein the first polymorphic variant of FLT3 is T227 and the second polymorphic variant of FLT3 is M227; or first polymorphic variant of FLT3 is M227 and the second polymorphic variant of FLT3 is T227.

Embodiment 126. The method of Embodiment 121, wherein the antigen is CLL-1.

Embodiment 127. The method of Embodiment 126, wherein the first polymorphic variant of CLL-1 is K224 and the second polymorphic variant of CLL-1 is Q244; or first polymorphic variant of CLL-1 is Q224 and the second polymorphic variant of CLL-1 is K244.

Embodiment 128. The method of any of Embodiments 114-127, wherein the subject is concurrently administered both the population of engineered immune effector cells and the population of hematopoietic cells.

Embodiment 128. The method of any of Embodiments 114-127, wherein the subject is sequentially administered the population of hematopoietic cells, and the population of engineered immune effector cells, mAb, or ADC.

Embodiment 130. The method of any of Embodiments 114-127, wherein the subject is sequentially administered the population of engineered immune effector cells, mAb, or ADC, and the population of hematopoietic cells.

Embodiment 131. The method of any of Embodiments 114-130, wherein the subject is treated with one or more conditioning regimens to deplete the subject of target cells bearing the first polymorphic variant of the antigen before administering of the hematopoietic cells.

Embodiment 132. The method of any of Embodiments 114-130, wherein the subject has already been conditioned with one or more conditioning regimens to deplete the subject of target cells bearing the first polymorphic variant of the antigen.

Embodiment 133. The method of any of Embodiments 114-12, wherein the hematopoietic cells are hematopoietic stem cells and/or hematopoietic progenitor cells.

Embodiment 134. The method of any of Embodiments 114-133, wherein the subject is administered a population of engineered immune effector cells that express a chimeric antigen receptor (CAR) that selectively binds the first polymorphic variant of the antigen on the surface of the target cell.

Embodiment 135. The method of Embodiment 134, wherein the engineered immune effector cells are derived from the subject (i.e., autologous) and the hematopoietic cells are derived from a donor (i.e., allogeneic).

Embodiment 136. The method of Embodiment 134, wherein the engineered immune effector cells and hematopoietic cells are derived from a single donor.

Embodiment 137. The method of Embodiment 134, wherein the engineered immune cells are derived from a first donor and hematopoietic cells are derived from a second donor.

Embodiment 138. The method of any of Embodiments 134-137, wherein the chimeric antigen receptor (CAR) comprises a polypeptide of any of Embodiments 1-69.

Embodiment 139. The method of Embodiment any of Embodiments 134-137, wherein the chimeric antigen receptor (CAR) comprises the scFv of Embodiment 70.

Embodiment 140. The method of any of Embodiments 134-137, wherein the chimeric antigen receptor (CAR) is a CAR of any of Embodiments 81-94.

Embodiment 141. The method of any of Embodiments 134-137, wherein the engineered immune effector cell is one of any of any of Embodiments 99-113.

Embodiment 142. The method of any of Embodiments 114-133, wherein the subject is administered a monoclonal antibody (mAb), or antigen-binding fragment thereof, that selectively binds the first polymorphic variant of the antigen on the surface of the target cell.

Embodiment 143. The method of Embodiment 142, wherein the monoclonal antibody (mAb) comprises a polypeptide of any of Embodiments 1-69.

Embodiment 144. The method of Embodiment 116, wherein the monoclonal antibody (mAb) is a mAb of any of Embodiments 71-77.

Embodiment 145. The method of any of Embodiments 114-133, wherein the subject is administered an antibody-drug conjugate (ADC) comprising a monoclonal antibody (mAb), or antigen-binding fragment thereof, that selectively binds the first polymorphic variant of the antigen on the surface of the target cell.

Embodiment 146. The method of any of Embodiments 142-145, wherein the mAb or ADC is administered prophylactically after transplant to prevent relapse.

Embodiment 147. The method of any of Embodiments 114-146, additionally comprising genotyping the subject and donor to ensure the HSC donor and patient express different variants of the target antigen.

Embodiment 148. The method of Embodiment 147, wherein the genotyping is done using either a protein- (FACS) or DNA- (PCR) based assay.

Embodiment 149. The method of Embodiment 147, wherein the patient is genotyped after relapse from transplant.

Embodiment 150. The method of Embodiment 147, wherein the patient is genotyped before transplant.

Embodiment 151. The method of Embodiment 147, wherein the hematopoietic cell donor is genotyped before hematopoietic cell transplant.

Embodiment 152. The method of any of Embodiments 137-147, wherein the immune effector cell donor is genotyped before transplant of the population of engineered immune effector cells that express the CAR.

Embodiment 153. The method of any of Embodiments 137-147, wherein the immune effector cell donor is genotyped before hematopoietic cell transplant.

Embodiment 154. A polypeptide which binds CD33, comprising:
three heavy chain variable (V_{H}) domain CDRs: HCDR1, HCDR2, and HCDR3; and/or
three light chain variable (V_{L}) domain CDRs: LCDR1, LCDR2, and LCDR3; wherein
HCDR1 comprises an amino acid sequence with at least 95% sequence identity to an amino acid sequence chosen from SEQ ID NOs: 705-7559 and 1979-1981;
HCDR2 comprises an amino acid sequence with at least 95% sequence identity to an amino acid sequence chosen from SEQ ID NOs: 760-814 and 1982-1984;
HCDR3 comprises an amino acid sequence with at least 95% sequence identity to an amino acid sequence chosen from SEQ ID NOs: 815-869 and 1985-1987;
LCDR1 comprises an amino acid sequence with at least 95% sequence identity to an amino acid sequence chosen from SEQ ID NOs: 870-924 and 1988-1990;
LCDR2 comprises an amino acid sequence with at least 95% sequence identity to an amino acid sequence chosen from SEQ ID NOs: 925-979 and 1991-1993; and
LCDR3 comprises an amino acid sequence with at least 95% sequence identity to an amino acid sequence chosen from SEQ ID NOs: 980-1034 and 1994-1996.

Embodiment 155. The polypeptide of Embodiment 154, comprising a combination of HCDR1, HCDR2, HCDR3, LCDR1, LCDR2, and LCDR3, respectively, chosen from:
SEQ ID NO.s: 705, 760, 815, 870, 925, and 980;
SEQ ID NO.s: 706, 761, 816, 871, 926, and 981;
SEQ ID NO.s: 707, 762, 817, 872, 927, and 982;
SEQ ID NO.s: 708, 763, 818, 873, 928, and 983;
SEQ ID NO.s: 709, 764, 819, 874, 929, and 984;
SEQ ID NO.s: 710, 765, 820, 875, 930, and 985;
SEQ ID NO.s: 711, 766, 821, 876, 931, and 986;
SEQ ID NO.s: 712, 767, 822, 877, 932, and 987;
SEQ ID NO.s: 713, 768, 823, 878, 933, and 988;
SEQ ID NO.s: 714, 769, 824, 879, 934, and 989;
SEQ ID NO.s: 715, 770, 825, 880, 935, and 990;
SEQ ID NO.s: 716, 771, 826, 881, 936, and 991;
SEQ ID NO.s: 717, 772, 827, 882, 937, and 992;
SEQ ID NO.s: 718, 773, 828, 883, 938, and 993;
SEQ ID NO.s: 719, 774, 829, 884, 939, and 994;
SEQ ID NO.s: 720, 775, 830, 885, 940, and 995;
SEQ ID NO.s: 721, 776, 831, 886, 941, and 996;
SEQ ID NO.s: 722, 777, 832, 887, 942, and 997;
SEQ ID NO.s: 723, 778, 833, 888, 943, and 998;
SEQ ID NO.s: 724, 779, 834, 889, 944, and 999;
SEQ ID NO.s: 725, 780, 835, 890, 945, and 1000;
SEQ ID NO.s: 726, 781, 836, 891, 946, and 1001;
SEQ ID NO.s: 727, 782, 837, 892, 947, and 1002;
SEQ ID NO.s: 728, 783, 838, 893, 948, and 1003;
SEQ ID NO.s: 729, 784, 839, 894, 949, and 1004;
SEQ ID NO.s: 730, 785, 840, 895, 950, and 1005;
SEQ ID NO.s: 731, 786, 841, 896, 951, and 1006;
SEQ ID NO.s: 732, 787, 842, 897, 952, and 1007;
SEQ ID NO.s: 733, 788, 843, 898, 953, and 1008;
SEQ ID NO.s: 734, 789, 844, 899, 954, and 1009;
SEQ ID NO.s: 735, 790, 845, 900, 955, and 1010;
SEQ ID NO.s: 736, 791, 846, 901, 956, and 1011;
SEQ ID NO.s: 737, 792, 847, 902, 957, and 1012;
SEQ ID NO.s: 738, 793, 848, 903, 958, and 1013;
SEQ ID NO.s: 739, 794, 849, 904, 959, and 1014;
SEQ ID NO.s: 740, 795, 850, 905, 960, and 1015;
SEQ ID NO.s: 741, 796, 851, 906, 961, and 1016;
SEQ ID NO.s: 742, 797, 852, 907, 962, and 1017;
SEQ ID NO.s: 743, 798, 853, 908, 963, and 1018;
SEQ ID NO.s: 744, 799, 854, 909, 964, and 1019;
SEQ ID NO.s: 745, 800, 855, 910, 965, and 1020;
SEQ ID NO.s: 746, 801, 856, 911, 966, and 1021;
SEQ ID NO.s: 747, 802, 857, 912, 967, and 1022;
SEQ ID NO.s: 748, 803, 858, 913, 968, and 1023;
SEQ ID NO.s: 749, 804, 859, 914, 969, and 1024;
SEQ ID NO.s: 750, 805, 860, 915, 970, and 1025;
SEQ ID NO.s: 751, 806, 861, 916, 971, and 1026;
SEQ ID NO.s: 752, 807, 862, 917, 972, and 1027;
SEQ ID NO.s: 753, 808, 863, 918, 973, and 1028;
SEQ ID NO.s: 754, 809, 864, 919, 974, and 1029;
SEQ ID NO.s: 755, 810, 865, 920, 975, and 1030;
SEQ ID NO.s: 756, 811, 866, 921, 976, and 1031;
SEQ ID NO.s: 757, 812, 867, 922, 977, and 1032;
SEQ ID NO.s: 758, 813, 868, 923, 978, and 1033;
SEQ ID NO.s: 759, 814, 869, 924, 979, and 1034;
SEQ ID NO.s: 1979, 1982, 1985, 1988, 1991, and 1994;
SEQ ID NO.s: 1980, 1983, 1986, 1989, 1992, and 1995; and
SEQ ID NO.s: 1981, 1984, 1987, 1990, 1993, and 1996;
or a combination of HCDR1, HCDR2, HCDR3, LCDR1, LCDR2, and LCDR3 with at least 95% sequence identity to the foregoing.

Embodiment 156. The polypeptide of Embodiment 154, comprising a V_{H} domain and V_{L} domain, wherein:
the V_{H} domain comprises an amino acid sequence with at least 95% sequence identity to an amino acid sequence chosen from SEQ ID NOs: 1035-1089 and 1997-1999; and/or
the V_{L} domain comprises an amino acid sequence with at least 95% sequence identity to an amino acid sequence chosen from SEQ ID NOs: 1090-1144 and 2000-2002.

Embodiment 157. The polypeptide of Embodiment 156, comprising a combination of V_{H} and V_{L} domains chosen from:
SEQ ID NO.s: 1035 and 1090;
SEQ ID NO.s: 1036 and 1091;
SEQ ID NO.s: 1037 and 1092;
SEQ ID NO.s: 1038 and 1093;
SEQ ID NO.s: 1039 and 1094;
SEQ ID NO.s: 1040 and 1095;
SEQ ID NO.s: 1041 and 1096;
SEQ ID NO.s: 1042 and 1097;
SEQ ID NO.s: 1043 and 1098;
SEQ ID NO.s: 1044 and 1099;
SEQ ID NO.s: 1045 and 1100;
SEQ ID NO.s: 1046 and 1101;
SEQ ID NO.s: 1047 and 1102;
SEQ ID NO.s: 1048 and 1103;
SEQ ID NO.s: 1049 and 1104;
SEQ ID NO.s: 1050 and 1105;
SEQ ID NO.s: 1051 and 1106;
SEQ ID NO.s: 1052 and 1107;
SEQ ID NO.s: 1053 and 1108;
SEQ ID NO.s: 1054 and 1109;
SEQ ID NO.s: 1055 and 1110;
SEQ ID NO.s: 1056 and 1111;
SEQ ID NO.s: 1057 and 1112;
SEQ ID NO.s: 1058 and 1113;
SEQ ID NO.s: 1059 and 1114;
SEQ ID NO.s: 1060 and 1115;
SEQ ID NO.s: 1061 and 1116;
SEQ ID NO.s: 1062 and 1117;
SEQ ID NO.s: 1063 and 1118;
SEQ ID NO.s: 1064 and 1119;
SEQ ID NO.s: 1065 and 1120;
SEQ ID NO.s: 1066 and 1121;
SEQ ID NO.s: 1067 and 1122;
SEQ ID NO.s: 1068 and 1123;
SEQ ID NO.s: 1069 and 1124;
SEQ ID NO.s: 1070 and 1125;
SEQ ID NO.s: 1071 and 1126;
SEQ ID NO.s: 1072 and 1127;
SEQ ID NO.s: 1073 and 1128;
SEQ ID NO.s: 1074 and 1129;
SEQ ID NO.s: 1075 and 1130;
SEQ ID NO.s: 1076 and 1131;
SEQ ID NO.s: 1077 and 1132;
SEQ ID NO.s: 1078 and 1133;
SEQ ID NO.s: 1079 and 1134;
SEQ ID NO.s: 1080 and 1135;
SEQ ID NO.s: 1081 and 1136;
SEQ ID NO.s: 1082 and 1137;
SEQ ID NO.s: 1083 and 1138;
SEQ ID NO.s: 1084 and 1139;
SEQ ID NO.s: 1085 and 1140;
SEQ ID NO.s: 1086 and 1141;
SEQ ID NO.s: 1087 and 1142;
SEQ ID NO.s: 1088 and 1143;
SEQ ID NO.s: 1089 and 1144;
SEQ ID NO.s: 1997 and 2000;
SEQ ID NO.s: 1998 and 2001; and
SEQ ID NO.s: 1999 and 2002;
or a combination of V_{H} and V_{L} domains with at least 95% sequence identity to the foregoing.

Embodiment 158. A polypeptide which binds CLL-1, comprising:
three heavy chain variable (V_{H}) domain CDRs: HCDR1, HCDR2, and HCDR3; and/or
three light chain variable (V_{L}) domain CDRs: LCDR1, LCDR2, and LCDR3; wherein
HCDR1 comprises an amino acid sequence with at least 95% sequence identity to an amino acid sequence chosen from SEQ ID NOs: 1145-1191 and 2003-2009;
HCDR2 comprises an amino acid sequence with at least 95% sequence identity to an amino acid sequence chosen from SEQ ID NOs: 1192-1238 and 2010-2016;
HCDR3 comprises an amino acid sequence with at least 95% sequence identity to an amino acid sequence chosen from SEQ ID NOs: 1239-1285 and 2017-2023;
LCDR1 comprises an amino acid sequence with at least 95% sequence identity to an amino acid sequence chosen from SEQ ID NOs: 1286-1332 and 2024-2030;
LCDR2 comprises an amino acid sequence with at least 95% sequence identity to an amino acid sequence chosen from SEQ ID NOs: 1333-1379 and 2031-2037; and
LCDR3 comprises an amino acid sequence with at least 95% sequence identity to an amino acid sequence chosen from SEQ ID NOs: 1380-1426 and 2038-2044.

Embodiment 159. The polypeptide of Embodiment 158, comprising a combination of HCDR1, HCDR2, HCDR3, LCDR1, LCDR2, and LCDR3, respectively, chosen from:
SEQ ID NO.s: 1145, 1192, 1239, 1286, 1333, and 1380;
SEQ ID NO.s: 1146, 1193, 1240, 1287, 1334, and 1381;
SEQ ID NO.s: 1147, 1194, 1241, 1288, 1335, and 1382;
SEQ ID NO.s: 1148, 1195, 1242, 1289, 1336, and 1383;
SEQ ID NO.s: 1149, 1196, 1243, 1290, 1337, and 1384;
SEQ ID NO.s: 1150, 1197, 1244, 1291, 1338, and 1385;
SEQ ID NO.s: 1151, 1198, 1245, 1292, 1339, and 1386;
SEQ ID NO.s: 1152, 1199, 1246, 1293, 1340, and 1387;
SEQ ID NO.s: 1153, 1200, 1247, 1294, 1341, and 1388;
SEQ ID NO.s: 1154, 1201, 1248, 1295, 1342, and 1389;
SEQ ID NO.s: 1155, 1202, 1249, 1296, 1343, and 1390;
SEQ ID NO.s: 1156, 1203, 1250, 1297, 1344, and 1391;
SEQ ID NO.s: 1157, 1204, 1251, 1298, 1345, and 1392;
SEQ ID NO.s: 1158, 1205, 1252, 1299, 1346, and 1393;
SEQ ID NO.s: 1159, 1206, 1253, 1300, 1347, and 1394;
SEQ ID NO.s: 1160, 1207, 1254, 1301, 1348, and 1395;
SEQ ID NO.s: 1161, 1208, 1255, 1302, 1349, and 1396;
SEQ ID NO.s: 1162, 1209, 1256, 1303, 1350, and 1397;
SEQ ID NO.s: 1163, 1210, 1257, 1304, 1351, and 1398;
SEQ ID NO.s: 1164, 1211, 1258, 1305, 1352, and 1399;
SEQ ID NO.s: 1165, 1212, 1259, 1306, 1353, and 1400;
SEQ ID NO.s: 1166, 1213, 1260, 1307, 1354, and 1401;
SEQ ID NO.s: 1167, 1214, 1261, 1308, 1355, and 1402;
SEQ ID NO.s: 1168, 1215, 1262, 1309, 1356, and 1403;
SEQ ID NO.s: 1169, 1216, 1263, 1310, 1357, and 1404;
SEQ ID NO.s: 1170, 1217, 1264, 1311, 1358, and 1405;
SEQ ID NO.s: 1171, 1218, 1265, 1312, 1359, and 1406;
SEQ ID NO.s: 1172, 1219, 1266, 1313, 1360, and 1407;
SEQ ID NO.s: 1173, 1220, 1267, 1314, 1361, and 1408;
SEQ ID NO.s: 1174, 1221, 1268, 1315, 1362, and 1409;
SEQ ID NO.s: 1175, 1222, 1269, 1316, 1363, and 1410;
SEQ ID NO.s: 1176, 1223, 1270, 1317, 1364, and 1411;
SEQ ID NO.s: 1177, 1224, 1271, 1318, 1365, and 1412;
SEQ ID NO.s: 1178, 1225, 1272, 1319, 1366, and 1413;
SEQ ID NO.s: 1179, 1226, 1273, 1320, 1367, and 1414;
SEQ ID NO.s: 1180, 1227, 1274, 1321, 1368, and 1415;
SEQ ID NO.s: 1181, 1228, 1275, 1322, 1369, and 1416;
SEQ ID NO.s: 1182, 1229, 1276, 1323, 1370, and 1417;
SEQ ID NO.s: 1183, 1230, 1277, 1324, 1371, and 1418;
SEQ ID NO.s: 1184, 1231, 1278, 1325, 1372, and 1419;
SEQ ID NO.s: 1185, 1232, 1279, 1326, 1373, and 1420;
SEQ ID NO.s: 1186, 1233, 1280, 1327, 1374, and 1421;
SEQ ID NO.s: 1187, 1234, 1281, 1328, 1375, and 1422;
SEQ ID NO.s: 1188, 1235, 1282, 1329, 1376, and 1423;
SEQ ID NO.s: 1189, 1236, 1283, 1330, 1377, and 1424;
SEQ ID NO.s: 1190, 1237, 1284, 1331, 1378, and 1425;
SEQ ID NO.s: 1191, 1238, 1285, 1332, 1379, and 1426;
SEQ ID NO.s: 2003, 2010, 2017, 2024, 2031, and 2038;
SEQ ID NO.s: 2004, 2011, 2018, 2025, 2032, and 2039;
SEQ ID NO.s: 2005, 2012, 2019, 2026, 2033, and 2040;
SEQ ID NO.s: 2006, 2013, 2020, 2027, 2034, and 2041;
SEQ ID NO.s: 2007, 2014, 2021, 2028, 2035, and 2042;
SEQ ID NO.s: 2008, 2015, 2022, 2029, 2036, and 2043; and
SEQ ID NO.s: 2009, 2016, 2023, 2030, 2037, and 2044;
or a combination of HCDR1, HCDR2, HCDR3, LCDR1, LCDR2, and LCDR3 with at least 95% sequence identity to the foregoing.

Embodiment 160. The polypeptide of Embodiment 158, comprising a V_{H} domain and V_{L} domain, wherein:
the V_{H} domain comprises an amino acid sequence with at least 95% sequence identity to an amino acid sequence chosen from SEQ ID NOs: 1427-1473 and 2045-2051; and/or
the V_{L} domain comprises an amino acid sequence with at least 95% sequence identity to an amino acid sequence chosen from SEQ ID NOs: 1474-1520 and 2052-2058.

Embodiment 161. The polypeptide of Embodiment 160, comprising a combination of V_{H} and V_{L} domains chosen from:
SEQ ID NO.s: 1427 and 1474;
SEQ ID NO.s: 1428 and 1475;
SEQ ID NO.s: 1429 and 1476;
SEQ ID NO.s: 1430 and 1477;
SEQ ID NO.s: 1431 and 1478;
SEQ ID NO.s: 1432 and 1479;
SEQ ID NO.s: 1433 and 1480;
SEQ ID NO.s: 1434 and 1481;
SEQ ID NO.s: 1435 and 1482;
SEQ ID NO.s: 1436 and 1483;
SEQ ID NO.s: 1437 and 1484;
SEQ ID NO.s: 1438 and 1485;
SEQ ID NO.s: 1439 and 1486;
SEQ ID NO.s: 1440 and 1487;
SEQ ID NO.s: 1441 and 1488;
SEQ ID NO.s: 1442 and 1489;
SEQ ID NO.s: 1443 and 1490;
SEQ ID NO.s: 1444 and 1491;
SEQ ID NO.s: 1445 and 1492;
SEQ ID NO.s: 1446 and 1493;
SEQ ID NO.s: 1447 and 1494;
SEQ ID NO.s: 1448 and 1495;
SEQ ID NO.s: 1449 and 1496;
SEQ ID NO.s: 1450 and 1497;
SEQ ID NO.s: 1451 and 1498;
SEQ ID NO.s: 1452 and 1499;
SEQ ID NO.s: 1453 and 1500;
SEQ ID NO.s: 1454 and 1501;
SEQ ID NO.s: 1455 and 1502;
SEQ ID NO.s: 1456 and 1503;
SEQ ID NO.s: 1457 and 1504;
SEQ ID NO.s: 1458 and 1505;
SEQ ID NO.s: 1459 and 1506;
SEQ ID NO.s: 1460 and 1507;
SEQ ID NO.s: 1461 and 1508;
SEQ ID NO.s: 1462 and 1509;
SEQ ID NO.s: 1463 and 1510;
SEQ ID NO.s: 1464 and 1511;
SEQ ID NO.s: 1465 and 1512;
SEQ ID NO.s: 1466 and 1513;
SEQ ID NO.s: 1467 and 1514;
SEQ ID NO.s: 1468 and 1515;
SEQ ID NO.s: 1469 and 1516;
SEQ ID NO.s: 1470 and 1517;
SEQ ID NO.s: 1471 and 1518;
SEQ ID NO.s: 1472 and 1519;
SEQ ID NO.s: 1473 and 1520;
SEQ ID NO.s: 2045 and 2052;
SEQ ID NO.s: 2046 and 2053;
SEQ ID NO.s: 2047 and 2054;
SEQ ID NO.s: 2048 and 2055;
SEQ ID NO.s: 2049 and 2056;
SEQ ID NO.s: 2050 and 2057; and
SEQ ID NO.s: 2051 and 2058;
or a combination of V_{H} and V_{L} domains with at least 95% sequence identity to the foregoing.

Embodiment 162. A polypeptide which binds FLT3, comprising: three heavy chain variable (V_{H}) domain CDRs: HCDR1, HCDR2, and HCDR3; and/or three light chain variable (V_{L}) domain CDRs: LCDR1, LCDR2, and LCDR3; wherein
HCDR1 comprises an amino acid sequence with at least 95% sequence identity to an amino acid sequence chosen from SEQ ID NOs: 2059-2152;
HCDR2 comprises an amino acid sequence with at least 95% sequence identity to an amino acid sequence chosen from SEQ ID NOs: 2153-2246;
HCDR3 comprises an amino acid sequence with at least 95% sequence identity to an amino acid sequence chosen from SEQ ID NOs: 2247-2340;
LCDR1 comprises an amino acid sequence with at least 95% sequence identity to an amino acid sequence chosen from SEQ ID NOs: 2341-2434;
LCDR2 comprises an amino acid sequence with at least 95% sequence identity to an amino acid sequence chosen from SEQ ID NOs: 2435-2528; and
LCDR3 comprises an amino acid sequence with at least 95% sequence identity to an amino acid sequence chosen from SEQ ID NOs: 2529-2622.

Embodiment 163. The polypeptide of Embodiment 162, comprising a combination of HCDR1, HCDR2, HCDR3, LCDR1, LCDR2, and LCDR3, respectively, chosen from:
SEQ ID NO.s: 2059, 2153, 2247, 2341, 2435, and 2529;
SEQ ID NO.s: 2060, 2154, 2248, 2342, 2436, and 2530;
SEQ ID NO.s: 2061, 2155, 2249, 2343, 2437, and 2531;
SEQ ID NO.s: 2062, 2156, 2250, 2344, 2438, and 2532;
SEQ ID NO.s: 2063, 2157, 2251, 2345, 2439, and 2533;
SEQ ID NO.s: 2064, 2158, 2252, 2346, 2440, and 2534;
SEQ ID NO.s: 2065, 2159, 2253, 2347, 2441, and 2535;
SEQ ID NO.s: 2066, 2160, 2254, 2348, 2442, and 2536;
SEQ ID NO.s: 2067, 2161, 2255, 2349, 2443, and 2537;
SEQ ID NO.s: 2068, 2162, 2256, 2350, 2444, and 2538;
SEQ ID NO.s: 2069, 2163, 2257, 2351, 2445, and 2539;
SEQ ID NO.s: 2070, 2164, 2258, 2352, 2446, and 2540;
SEQ ID NO.s: 2071, 2165, 2259, 2353, 2447, and 2541;
SEQ ID NO.s: 2072, 2166, 2260, 2354, 2448, and 2542;
SEQ ID NO.s: 2073, 2167, 2261, 2355, 2449, and 2543;
SEQ ID NO.s: 2074, 2168, 2262, 2356, 2450, and 2544;
SEQ ID NO.s: 2075, 2169, 2263, 2357, 2451, and 2545;
SEQ ID NO.s: 2076, 2170, 2264, 2358, 2452, and 2546;
SEQ ID NO.s: 2077, 2171, 2265, 2359, 2453, and 2547;
SEQ ID NO.s: 2078, 2172, 2266, 2360, 2454, and 2548;
SEQ ID NO.s: 2079, 2173, 2267, 2361, 2455, and 2549;
SEQ ID NO.s: 2080, 2174, 2268, 2362, 2456, and 2550;
SEQ ID NO.s: 2081, 2175, 2269, 2363, 2457, and 2551;
SEQ ID NO.s: 2082, 2176, 2270, 2364, 2458, and 2552;
SEQ ID NO.s: 2083, 2177, 2271, 2365, 2459, and 2553;
SEQ ID NO.s: 2084, 2178, 2272, 2366, 2460, and 2554;
SEQ ID NO.s: 2085, 2179, 2273, 2367, 2461, and 2555;
SEQ ID NO.s: 2086, 2180, 2274, 2368, 2462, and 2556;
SEQ ID NO.s: 2087, 2181, 2275, 2369, 2463, and 2557;
SEQ ID NO.s: 2088, 2182, 2276, 2370, 2464, and 2558;
SEQ ID NO.s: 2089, 2183, 2277, 2371, 2465, and 2559;
SEQ ID NO.s: 2090, 2184, 2278, 2372, 2466, and 2560;
SEQ ID NO.s: 2091, 2185, 2279, 2373, 2467, and 2561;
SEQ ID NO.s: 2092, 2186, 2280, 2374, 2468, and 2562;
SEQ ID NO.s: 2093, 2187, 2281, 2375, 2469, and 2563;
SEQ ID NO.s: 2094, 2188, 2282, 2376, 2470, and 2564;
SEQ ID NO.s: 2095, 2189, 2283, 2377, 2471, and 2565;
SEQ ID NO.s: 2096, 2190, 2284, 2378, 2472, and 2566;
SEQ ID NO.s: 2097, 2191, 2285, 2379, 2473, and 2567;
SEQ ID NO.s: 2098, 2192, 2286, 2380, 2474, and 2568;
SEQ ID NO.s: 2099, 2193, 2287, 2381, 2475, and 2569;
SEQ ID NO.s: 2100, 2194, 2288, 2382, 2476, and 2570;
SEQ ID NO.s: 2101, 2195, 2289, 2383, 2477, and 2571;
SEQ ID NO.s: 2102, 2196, 2290, 2384, 2478, and 2572;
SEQ ID NO.s: 2103, 2197, 2291, 2385, 2479, and 2573;
SEQ ID NO.s: 2104, 2198, 2292, 2386, 2480, and 2574;
SEQ ID NO.s: 2105, 2199, 2293, 2387, 2481, and 2575;
SEQ ID NO.s: 2106, 2200, 2294, 2388, 2482, and 2576;
SEQ ID NO.s: 2107, 2201, 2295, 2389, 2483, and 2577;
SEQ ID NO.s: 2108, 2202, 2296, 2390, 2484, and 2578;
SEQ ID NO.s: 2109, 2203, 2297, 2391, 2485, and 2579;
SEQ ID NO.s: 2110, 2204, 2298, 2392, 2486, and 2580;
SEQ ID NO.s: 2111, 2205, 2299, 2393, 2487, and 2581;
SEQ ID NO.s: 2112, 2206, 2300, 2394, 2488, and 2582;
SEQ ID NO.s: 2113, 2207, 2301, 2395, 2489, and 2583;
SEQ ID NO.s: 2114, 2208, 2302, 2396, 2490, and 2584;
SEQ ID NO.s: 2115, 2209, 2303, 2397, 2491, and 2585;
SEQ ID NO.s: 2116, 2210, 2304, 2398, 2492, and 2586;
SEQ ID NO.s: 2117, 2211, 2305, 2399, 2493, and 2587;
SEQ ID NO.s: 2118, 2212, 2306, 2400, 2494, and 2588;
SEQ ID NO.s: 2119, 2213, 2307, 2401, 2495, and 2589;
SEQ ID NO.s: 2120, 2214, 2308, 2402, 2496, and 2590;
SEQ ID NO.s: 2121, 2215, 2309, 2403, 2497, and 2591;
SEQ ID NO.s: 2122, 2216, 2310, 2404, 2498, and 2592;
SEQ ID NO.s: 2123, 2217, 2311, 2405, 2499, and 2593;
SEQ ID NO.s: 2124, 2218, 2312, 2406, 2500, and 2594;
SEQ ID NO.s: 2125, 2219, 2313, 2407, 2501, and 2595;
SEQ ID NO.s: 2126, 2220, 2314, 2408, 2502, and 2596;
SEQ ID NO.s: 2127, 2221, 2315, 2409, 2503, and 2597;
SEQ ID NO.s: 2128, 2222, 2316, 2410, 2504, and 2598;
SEQ ID NO.s: 2129, 2223, 2317, 2411, 2505, and 2599;
SEQ ID NO.s: 2130, 2224, 2318, 2412, 2506, and 2600;
SEQ ID NO.s: 2131, 2225, 2319, 2413, 2507, and 2601;
SEQ ID NO.s: 2132, 2226, 2320, 2414, 2508, and 2602;
SEQ ID NO.s: 2133, 2227, 2321, 2415, 2509, and 2603;
SEQ ID NO.s: 2134, 2228, 2322, 2416, 2510, and 2604;
SEQ ID NO.s: 2135, 2229, 2323, 2417, 2511, and 2605;
SEQ ID NO.s: 2136, 2230, 2324, 2418, 2512, and 2606;
SEQ ID NO.s: 2137, 2231, 2325, 2419, 2513, and 2607;
SEQ ID NO.s: 2138, 2232, 2326, 2420, 2514, and 2608;
SEQ ID NO.s: 2139, 2233, 2327, 2421, 2515, and 2609;
SEQ ID NO.s: 2140, 2234, 2328, 2422, 2516, and 2610;
SEQ ID NO.s: 2141, 2235, 2329, 2423, 2517, and 2611;
SEQ ID NO.s: 2142, 2236, 2330, 2424, 2518, and 2612;
SEQ ID NO.s: 2143, 2237, 2331, 2425, 2519, and 2613;
SEQ ID NO.s: 2144, 2238, 2332, 2426, 2520, and 2614;
SEQ ID NO.s: 2145, 2239, 2333, 2427, 2521, and 2615;
SEQ ID NO.s: 2146, 2240, 2334, 2428, 2522, and 2616;
SEQ ID NO.s: 2147, 2241, 2335, 2429, 2523, and 2617;
SEQ ID NO.s: 2148, 2242, 2336, 2430, 2524, and 2618;
SEQ ID NO.s: 2149, 2243, 2337, 2431, 2525, and 2619;
SEQ ID NO.s: 2150, 2244, 2338, 2432, 2526, and 2620;
SEQ ID NO.s: 2151, 2245, 2339, 2433, 2527, and 2621; and
SEQ ID NO.s: 2152, 2246, 2340, 2434, 2528, and 2622;
or a combination of HCDR1, HCDR2, HCDR3, LCDR1, LCDR2, and LCDR3 with at least 95% sequence identity to the foregoing.

Embodiment 164. The polypeptide of Embodiment 162, comprising a V_{H} domain and V_{L} domain, wherein:
the V_{H} domain comprises an amino acid sequence with at least 95% sequence identity to an amino acid sequence chosen from SEQ ID NOs: 2623-2716; and/or
the V_{L} domain comprises an amino acid sequence with at least 95% sequence identity to an amino acid sequence chosen from SEQ ID NOs: 2717-2810.

Embodiment 165. The polypeptide of Embodiment 164, comprising a combination of V_{H} and V_{L} domains chosen from:
SEQ ID NO.s: 2623 and 2717;
SEQ ID NO.s: 2624 and 2718;
SEQ ID NO.s: 2625 and 2719;
SEQ ID NO.s: 2626 and 2720;
SEQ ID NO.s: 2627 and 2721;
SEQ ID NO.s: 2628 and 2722;
SEQ ID NO.s: 2629 and 2723;
SEQ ID NO.s: 2630 and 2724;
SEQ ID NO.s: 2631 and 2725;
SEQ ID NO.s: 2632 and 2726;
SEQ ID NO.s: 2633 and 2727;
SEQ ID NO.s: 2634 and 2728;
SEQ ID NO.s: 2635 and 2729;
SEQ ID NO.s: 2636 and 2730;
SEQ ID NO.s: 2637 and 2731;
SEQ ID NO.s: 2638 and 2732;
SEQ ID NO.s: 2639 and 2733;
SEQ ID NO.s: 2640 and 2734;
SEQ ID NO.s: 2641 and 2735;
SEQ ID NO.s: 2642 and 2736;
SEQ ID NO.s: 2643 and 2737;
SEQ ID NO.s: 2644 and 2738;
SEQ ID NO.s: 2645 and 2739;
SEQ ID NO.s: 2646 and 2740;
SEQ ID NO.s: 2647 and 2741;
SEQ ID NO.s: 2648 and 2742;
SEQ ID NO.s: 2649 and 2743;
SEQ ID NO.s: 2650 and 2744;
SEQ ID NO.s: 2651 and 2745;
SEQ ID NO.s: 2652 and 2746;
SEQ ID NO.s: 2653 and 2747;
SEQ ID NO.s: 2654 and 2748;
SEQ ID NO.s: 2655 and 2749;
SEQ ID NO.s: 2656 and 2750;
SEQ ID NO.s: 2657 and 2751;
SEQ ID NO.s: 2658 and 2752;
SEQ ID NO.s: 2659 and 2753;
SEQ ID NO.s: 2660 and 2754;
SEQ ID NO.s: 2661 and 2755;
SEQ ID NO.s: 2662 and 2756;
SEQ ID NO.s: 2663 and 2757;
SEQ ID NO.s: 2664 and 2758;
SEQ ID NO.s: 2665 and 2759;
SEQ ID NO.s: 2666 and 2760;
SEQ ID NO.s: 2667 and 2761;
SEQ ID NO.s: 2668 and 2762;
SEQ ID NO.s: 2669 and 2763;
SEQ ID NO.s: 2670 and 2764;
SEQ ID NO.s: 2671 and 2765;
SEQ ID NO.s: 2672 and 2766;
SEQ ID NO.s: 2673 and 2767;
SEQ ID NO.s: 2674 and 2768;
SEQ ID NO.s: 2675 and 2769;
SEQ ID NO.s: 2676 and 2770;
SEQ ID NO.s: 2677 and 2771;
SEQ ID NO.s: 2678 and 2772;
SEQ ID NO.s: 2679 and 2773;
SEQ ID NO.s: 2680 and 2774;
SEQ ID NO.s: 2681 and 2775;
SEQ ID NO.s: 2682 and 2776;
SEQ ID NO.s: 2683 and 2777;
SEQ ID NO.s: 2684 and 2778;
SEQ ID NO.s: 2685 and 2779;
SEQ ID NO.s: 2686 and 2780;
SEQ ID NO.s: 2687 and 2781;
SEQ ID NO.s: 2688 and 2782;
SEQ ID NO.s: 2689 and 2783;
SEQ ID NO.s: 2690 and 2784;
SEQ ID NO.s: 2691 and 2785;
SEQ ID NO.s: 2692 and 2786;
SEQ ID NO.s: 2693 and 2787;
SEQ ID NO.s: 2694 and 2788;
SEQ ID NO.s: 2695 and 2789;
SEQ ID NO.s: 2696 and 2790;
SEQ ID NO.s: 2697 and 2791;
SEQ ID NO.s: 2698 and 2792;
SEQ ID NO.s: 2699 and 2793;
SEQ ID NO.s: 2700 and 2794;
SEQ ID NO.s: 2701 and 2795;
SEQ ID NO.s: 2702 and 2796;
SEQ ID NO.s: 2703 and 2797;
SEQ ID NO.s: 2704 and 2798;
SEQ ID NO.s: 2705 and 2799;
SEQ ID NO.s: 2706 and 2800;
SEQ ID NO.s: 2707 and 2801;
SEQ ID NO.s: 2708 and 2802;
SEQ ID NO.s: 2709 and 2803;
SEQ ID NO.s: 2710 and 2804;
SEQ ID NO.s: 2711 and 2805;
SEQ ID NO.s: 2712 and 2806;
SEQ ID NO.s: 2713 and 2807;
SEQ ID NO.s: 2714 and 2808;
SEQ ID NO.s: 2715 and 2809; and
SEQ ID NO.s: 2716 and 2810;
or a combination of V_{H} and V_{L} domains with at least 95% sequence identity to the foregoing.

Embodiment 165. The polypeptide of any of Embodiments 154-164, wherein
the HCDR1, HCDR2, and HCDR3 and/or the LCDR1, LCDR2, and LCDR3, or
the V_{H} and/or V_{L} domains,
   have at least 97%, 98% or 99% sequence identity to the recited amino acid sequences.

Embodiment 166. A single-chain variable fragment (scFv) comprising the polypeptide of any of Embodiments 154-164.

Embodiment 167. A monoclonal antibody (mAb), or an antigen-binding fragment thereof, comprising the polypeptide of any of Embodiments 154-164.

Embodiment 168. The mAb, or antigen-binding fragment thereof, of Embodiment 167, wherein the mAb is of the IgG, IgM, or IgA isotype.

Embodiment 169. The mAb, or antigen-binding fragment thereof, of Embodiment 170, wherein the mAb is of the IgG1 isotype.

Embodiment 170. The mAb, or antigen-binding fragment thereof, of Embodiment 170, wherein the mAb is of the IgG3 isotype.

Embodiment 171. The mAb, or antigen-binding fragment thereof, of Embodiment 170, wherein the mAb is of the IgG4 isotype.

Embodiment 172. The mAb, or antigen-binding fragment thereof, of Embodiment 170, wherein the mAb is human or humanized.

Embodiment 173. An antibody-drug conjugate (ADC) comprising the mAb, or antigen-binding fragment thereof, of any of Embodiments 167-172.

Embodiment 174. The ADC of Embodiment 173, having Formula I:

Ab-(L-D)ₚ (I)

wherein:
Ab is an antibody comprising the polypeptide of any of Embodiments 1-43, or the antibody of any of Embodiments 45-51, or an antigen-binding fragment of either of the foregoing;
L is a linker;
D is a drug; and
p is about 1 to about 20.

Embodiment 175. The ADC of Embodiment 174, wherein D is chosen from saporin, MMAE, MMAF, DM1, and DM4.

Embodiment 176. A chimeric antigen receptor (CAR) comprising an extracellular ligand binding domain comprising a polypeptide of any one of Embodiments 1-69.

Embodiment 177. The CAR of Embodiment 176, additionally comprising:
a hinge domain;
a transmembrane domain;
optionally, one or more co-stimulatory domains; and
a cytoplasmic signaling domain.

Embodiment 178. The CAR of Embodiment 177, wherein the hinge domain is chosen from FcεRIIIa, CD8α, CD28 and IgG1.

Embodiment 179. The CAR of Embodiment 178, wherein the hinge domain is CD8α.

Embodiment 180. The CAR of any of Embodiments 177-179, wherein the transmembrane domain is chosen from alpha, beta or zeta chain of the T-cell receptor, CD28, CD3 epsilon, CD45, CD4, CD5, CD9, CD16, CD22, CD33, CD37, CD64, CDS0, CD86, CD134, CD137 and CD154.

Embodiment 181. The CAR of Embodiment 180, wherein the transmembrane domain is CD28.

Embodiment 182. The CAR of any of Embodiments 177-181, wherein the cytoplasmic signaling domain is chosen from CD8, CD3ζ, CD3δ, CD3γ, CD3δ, CD22, CD32, DAP10, DAP12, CD66d, CD79a, CD79b, FcγRIγ, FcγRIIIγ, FcεRIβ, FcεRIγ, FcRγ, FcRβ, and FcRε.

Embodiment 183. The CAR of Embodiment 182, wherein the cytoplasmic signaling domain is CD3ζ.

Embodiment 184. The CAR of any of Embodiments 177-183, wherein one co-stimulatory domain is chosen from 4-1BB, CD28, and ICOS.

Embodiment 185. The CAR of Embodiment 184, wherein the costimulatory domain is CD28.

Embodiment 186. The CAR of Embodiment 184, wherein the costimulatory domain is 4-1BB.

Embodiment 187. The CAR of Embodiment 184, comprising two or more costimulatory domains.

Embodiment 188. The CAR of Embodiment 184, wherein two of the costimulatory domains are CD28 and 4-1BB.

Embodiment 189. A nucleotide sequence encoding any of the polypeptides, scFvs, mAbs, or CARs of any of Embodiments 154-188.

Embodiment 190. A vector comprising the nucleotide sequence of Embodiment 189.

Embodiment 191. The vector of Embodiment 190, wherein the vector is a lentiviral vector.

Embodiment 192. The vector of Embodiment 191, wherein the lentiviral vector comprises a VSVG domain.

Embodiment 193. An engineered immune effector cell expressing at the cell surface a CAR of any one of Embodiment 176-188.

Embodiment 194. The engineered immune effector cell of Embodiment 193, wherein the engineered immune effector cell expresses at the cell surface:
a first polymorphic variant of a human cancer cell antigen; and
a CAR that is selective for a second polymorphic over the first polymorphic variant of the antigen.

Embodiment 195. The engineered immune effector cell of Embodiment 193, wherein the cell is a primary cell.

Embodiment 196. The engineered immune effector cell of Embodiment 193, wherein the cell is derived from:
an induced pluripotent stem cell (iPSC);
cord blood;
peripheral blood; or
an immortalized cell line.

Embodiment 197. The engineered immune effector cell of Embodiment 196, wherein the immortalized cell line is NK-92.

Embodiment 198. The engineered immune cell of any of Embodiments 193-197, wherein the cell is chosen from a T cell, an natural killer (NK) cell, an invariant natural killer T (iNKT) cell, a macrophage, and a dendritic cell.

Embodiment 199. The engineered immune effector cell of Embodiment 198, wherein the cell is a T cell.

Embodiment 200. The engineered immune effector cell of Embodiment 199, wherein the T cell is chosen from an inflammatory T-lymphocyte, a cytotoxic T-lymphocyte, a regulatory T-lymphocyte, or a helper T-lymphocyte.

Embodiment 201. The engineered immune effector cell of Embodiment 199, wherein the engineered immune effector cell is deficient in a subunit of the T cell receptor complex.

Embodiment 202. The engineered immune effector cell of Embodiment 201, wherein the subunit of the T cell receptor complex is chosen from TCRα(TRAC), TCRβ, TCRδ, TCRγ, CD3ε, CD3γ, CD3δ, and CD3ζ.

Embodiment 203. The engineered immune effector cell of any of Embodiments 193-202, wherein the engineered immune effector cell is deficient in a cell surface protein that is the target of the CAR.

Embodiment 204. The engineered immune effector cell of Embodiment 198, wherein the engineered immune effector cell is an NK cell.

Embodiment 205. The engineered immune effector cell of Embodiment 204 wherein the engineered immune effector cell is a memory-like (ML) NK cell.

Embodiment 206. The engineered immune effector cell of Embodiment 205, wherein the engineered immune effector cell is a cytokine-induced memory-like (CIML) NK cell.

Embodiment 207. The engineered immune effector cell of Embodiment 198, wherein the engineered immune effector cell is an iNKT cell.

Embodiment 208. A method for treatment of cancer in a patient comprising administering to a cancer patient, a therapeutically effective amount of: a monoclonal antibody (mAb), or an antigen-binding fragment thereof, of any of Embodiments 167-170; an antibody-drug conjugate (ADC) of any of Embodiments 173-175; or an engineered immune effector cell of any of Embodiments 193-207.

Embodiment 209. The method of Embodiment 208, wherein the cancer is a hematologic malignancy.

Embodiment 210. The method of Embodiment 209, wherein the hematologic malignancy is multiple myeloma.

Embodiment 211. The method of Embodiment 210, wherein the hematologic malignancy is acute myeloid leukemia (AML).

### Polypeptides

Disclosed herein are polypeptides, such as monoclonal antibodies (mAbs) and functional fragments thereof, synthetic antigen-binding proteins such as single-chain variable fragments (scFvs), and chimeric antigen receptors (CARs), that can specifically recognize tumor-associated antigens (TAAs) on cancer cells, for example those that express CD33, FLT3, and CLL-1. In some embodiments, the mAbs, scFvs, or CARs recognize polymorphic variants of CD33, FLT3, and CLL-1 expressed on cancer cells; in some embodiments, they are selective for one polymorphic variant over other polymorphic variants. Also disclosed are immune effector cells, such as T cells, natural killer (NK) cells, and invariant natural killer T (iNKT) cells that are engineered to express CARs that specifically recognize the tumor-associated antigens (TAAs) CD33, FLT3, and CLL-1 or polymorphic variants of CD33, FLT3, and CLL-1. Also disclosed are methods for providing an anti-tumor immunity in a subject with CD33, FLT3, and CLL-1-expressing cancers using the disclosed monoclonal antibodies and immune effector cells which express CARs.

Antibodies that can be used in the disclosed compositions and methods include whole immunoglobulin (i.e., an intact antibody) of any class, fragments thereof, and, using the term more loosely, synthetic proteins containing at least the antigen binding variable domain of an antibody (e.g., single-chain variable fragments, scFvs). The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge. Human antibodies can also be produced in phage display libraries. The techniques of Cote et al. and Boerner et al. are also available for the preparation of human monoclonal antibodies.

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementarity determining region (CDR) of the recipient antibody are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin.

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, a humanized form of a non-human antibody (or an antigen-binding fragment thereof) is a chimeric antibody or fragment (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The embodiments of the disclosure include polypeptides, specifically monoclonal antibodies (mAbs), antigen-binding fragments thereof, synthetic antigen-binding proteins such as scFvs, and chimeric antigen receptors (CARs), which are defined by reference to structural characteristics, i.e., specific amino acid sequences of either the Complementarity-Determining Regions (CDRs), heavy chain or light chain variable domains (V_{H} or V_{L}), or full length heavy or light chains (HC or LC). The monoclonal antibodies or antigen binding fragments thereof of the disclosure bind to, e.g., CD33, FLT3, or CLL-1 or polymorphic variants thereof.

Also disclosed are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the non-modified antibody or antibody fragment.

Techniques can also be adapted for the production of synthetic single-chain antibodies (actually antibody-like fusion proteins) specific to an antigenic protein of the present disclosure. Methods for the production of single-chain antibodies are well known to those of skill in the art. A single-chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

The monoclonal antibodies or antigen binding fragments thereof of the disclosure, comprise at least one, usually at least three CDR sequences, in combination with framework sequences from a human variable region or as an isolated CDR peptide. In some embodiments, an antibody comprises at least one heavy chain comprising three heavy chain CDR sequences situated in a variable region framework, which may be a human or murine variable region framework, and at least one light chain comprising the three light chain CDR sequences provided herein situated in a variable region framework, which may be a murine or human variable region framework.

### Anti-CD33 Polypeptides

In some embodiments of the disclosure are provided anti-CD33 polypeptides, including mAbs, antigen binding fragments thereof, and synthetic fusion proteins such as single-chain variable fragments (scFvs), comprising one or more complementarity-determining regions (CDRs) which recognize and bind CD33. In some embodiments, the anti-CD33 polypeptides, including mAbs, antigen binding fragments thereof, and synthetic fusion proteins such as single-chain variable fragments (scFvs) selectively bind a first polymorphic variant of CD33 over a second polymorphic variant of CD33; or selectively binds the second polymorphic variant of CD33 over the first polymorphic variant. In some embodiments, the binding is at least 2-fold, 10-fold, or 30-fold selective.

In some embodiments, the first polymorphic variant of CD33 is R69 and the second polymorphic variant of CD33 is G69; or first polymorphic variant of CD33 is G69 and the second polymorphic variant of CD33 is R69.

In some embodiments of the disclosure are provided anti-CD33 polypeptides, including mAbs, antigen binding fragments thereof, and synthetic fusion proteins such as single-chain variable fragments (scFvs), comprising one or more complementarity-determining regions (CDRs) which recognize and bind CD33. Sequences of the CDRs and V_{H} and V_{L} domains for the anti-CD33 polypeptides described herein for binding CD33 are provided in Tables and Examples below.

Provided herein therefore, are a heavy chain variable (V_{H}) domain CDR1 (HCDR1), a V_{H} domain CDR2 (HCDR2), and a V_{H} domain CDR3 (HCDR3), and polypeptides comprising them, wherein the HCDR1 comprises an amino acid sequence chosen from any of SEQ ID NOs 1-25 and 201-217; HCDR2 comprises an amino acid sequence chosen from any of SEQ ID NOs 26-50 and 218-234; and HCDR3 comprises an amino acid sequence chosen from any of SEQ ID NOs 51-75 and 235-251. Also provided are a HCDR1, a HCDR2, and a HCDR3, and polypeptides comprising them, wherein the HCDR1 comprises an amino acid sequence chosen from any of SEQ ID NOs 1-25; HCDR2 comprises an amino acid sequence chosen from any of SEQ ID NOs 26-50; and HCDR3 comprises an amino acid sequence chosen from any of SEQ ID NOs 51-75. Also provided are HCDR1, a HCDR2, and a HCDR3, and polypeptides comprising them, wherein the HCDR1 comprises an amino acid sequence chosen from any of SEQ ID NOs 201-217; HCDR2 comprises an amino acid sequence chosen from any of SEQ ID NOs 218-234; and HCDR3 comprises an amino acid sequence chosen from any of SEQ ID NOs 235-251.

Also provided is a V_{H} domain comprising one or more of these CDRs. The V_{H} domain of the anti-CD33 mAb or antigen binding fragment thereof may comprise any one of the listed HCDR1 sequences in combination with any one of the HCDR2 sequences, and in combination with any one of the HCDR3 sequences. However, in certain embodiments, the provided HCDR1, HCDR2, and HCDR3 sequences are derived from a single common V_{H} domain, the examples of which are described herein.

The anti-CD33 mAbs, antigen binding fragments thereof, and synthetic antigen-binding proteins such as scFvs may additionally comprise a light chain variable (V_{L}) domain, which is paired with the V_{H} domain to form an CD33 antigen binding domain.

Provided herein therefore, are a light chain variable (V_{L}) domain CDR1 (LCDR1), a V_{L} domain CDR2 (LCDR2), and a V_{L} domain CDR3 (LCDR3), and polypeptides comprising them, wherein the LCDR1 comprises an amino acid sequence chosen from: SEQ ID NOs 76-100 and 252-268; LCDR2 comprises an amino acid sequence chosen from: SEQ ID NOs 101-125 and 269-285; and LCDR3 comprises an amino acid sequence chosen from: SEQ ID NOs 126-150 and 286-302. Also provided are a LCDR1, a LCDR2, and a LCDR3, and polypeptides comprising them, wherein the LCDR1 comprises an amino acid sequence chosen from any of SEQ ID NOs 76-100; LCDR2 comprises an amino acid sequence chosen from any of SEQ ID NOs 101-125; and LCDR3 comprises an amino acid sequence chosen from any of SEQ ID NOs 125-150. Also provided are a LCDR1, a LCDR2, and a LCDR3, and polypeptides comprising them, wherein the LCDR1 comprises an amino acid sequence chosen from any of SEQ ID NOs 252-268; LCDR2 comprises an amino acid sequence chosen from any of SEQ ID NOs 269-285; and LCDR3 comprises an amino acid sequence chosen from any of SEQ ID NOs 286-302.

Also provided is a V_{L} domain comprising one or more of these CDRs. The V_{L} domain of the anti-CD33 mAb, antigen binding fragment thereof, or synthetic antigen-binding protein such as an scFv may comprise any one of the listed LCDR1 sequences in combination with any one of the LCDR2 sequences, and in combination with any one of the LCDR3 sequences. However, in certain embodiments, the LCDR1, LCDR2, and LCDR3 sequences are derived from a single common V_{L} domain, examples of which are described herein.

Also provided are mAbs, antigen binding fragments thereof, and synthetic antigen-binding proteins such as scFvs comprising the CDRs, V_{H} domains, and/or V_{L} domain disclosed herein. Any given anti-CD33 mAb (and certain antigen-binding fragments thereof) or scFv comprising a V_{H} domain paired with a V_{L} domain will comprise a combination of six (6) CDRs: a V_{H} domain CDR1 (HCDR1), a V_{H} domain CDR2 (HCDR2), and a V_{H} domain CDR3 (HCDR3), a V_{L} domain CDR1 (LCDR1), a V_{L} domain CDR2 (LCDR2), and a V_{L} domain CDR3 (LCDR3). Although all combinations of six (6) CDRs chosen from the CDR amino acid sequences described above are permissible and within the scope of the disclosure, certain combinations of the six (6) CDRs are provided herein.

In some embodiments, the combination of the six (6) CDRs is chosen from the combinations recited in each of Polypeptide No.s 1-42. In some embodiments, the combination of the six (6) CDRs is chosen from the combinations recited in each of Polypeptide No.s 1-25. In some embodiments, the combination of the six (6) CDRs is chosen from the combinations recited in each of Polypeptide No.s 26-42.

In some embodiments, the anti-CD33 mAbs, antigen binding fragments thereof, and synthetic antigen-binding proteins such as scFvs comprise a V_{H} domain chosen from any of SEQ ID NOs 151-175 and 303-319, with amino acid sequences exhibiting at least 90% sequence identity, or at least 95%, 96% 97%, 98% or 99% sequence identity to one of the recited amino acid sequences. In some embodiments, the anti-CD33 mAbs, antigen binding fragments thereof, and synthetic antigen-binding proteins such as scFvs comprise a V_{H} domain chosen from any of SEQ ID NOs 151-175, with amino acid sequences exhibiting at least 90% sequence identity, or at least 95%, 96% 97%, 98% or 99% sequence identity to one of the recited amino acid sequences. In some embodiments, the anti-CD33 mAbs, antigen binding fragments thereof, and synthetic antigen-binding proteins such as scFvs comprise a V_{H} domain chosen from any of SEQ ID NOs 303-319, with amino acid sequences exhibiting at least 90% sequence identity, or at least 95%, 96% 97%, 98% or 99% sequence identity to one of the recited amino acid sequences.

Alternatively, or in addition, the anti-CD33 mAbs, antigen binding fragments thereof, and synthetic antigen-binding proteins such as scFvs comprise a V_{L} domain having an amino acid sequence chosen from any of SEQ ID NOs 176-200 and 320-336, and amino acid sequences exhibiting at least 90% sequence identity, or at least 95%, 96% 97%, 98% or 99% sequence identity to one of the recited amino acid sequences. In some embodiments, the anti-CD33 mAbs, antigen binding fragments thereof, and synthetic antigen-binding proteins such as scFvs comprise a V_{L} domain having an amino acid sequence chosen from any of SEQ ID NOs 176-200, and amino acid sequences exhibiting at least 90% sequence identity, or at least 95%, 96% 97%, 98% or 99% sequence identity to one of the recited amino acid sequences. In some embodiments, the anti-CD33 mAbs, antigen binding fragments thereof, and synthetic antigen-binding proteins such as scFvs comprise a V_{L} domain having an amino acid sequence chosen from any of SEQ ID NOs 320-336, and amino acid sequences exhibiting at least 90% sequence identity, or at least 95%, 96% 97%, 98% or 99% sequence identity to one of the recited amino acid sequences.

Although all possible pairing of V_{H} domains and V_{L} domains chosen from the V_{H} and V_{L} domain amino acid sequences listed above are permissible and within the scope of the disclosure, some embodiments provide certain combinations of V_{H} and V_{L} domains. Accordingly, in some embodiments, anti-CD33 mAbs, antigen binding fragments thereof, and synthetic antigen-binding proteins such as scFvs comprise a combination of a V_{H} domain and a V_{L} domain, wherein the combination is chosen from those recited in Polypeptide No.s 1-42, e.g.:
SEQ ID NO: 151 and SEQ ID NO: 176;
SEQ ID NO: 152 and SEQ ID NO: 177;
SEQ ID NO: 153 and SEQ ID NO: 178;
SEQ ID NO: 154 and SEQ ID NO: 179;
SEQ ID NO: 155 and SEQ ID NO: 180;
SEQ ID NO: 156 and SEQ ID NO: 181;
SEQ ID NO: 157 and SEQ ID NO: 182;
SEQ ID NO: 158 and SEQ ID NO: 183;
SEQ ID NO: 159 and SEQ ID NO: 184;
SEQ ID NO: 160 and SEQ ID NO: 185;
SEQ ID NO: 161 and SEQ ID NO: 186;
SEQ ID NO: 162 and SEQ ID NO: 187;
SEQ ID NO: 163 and SEQ ID NO: 188;
SEQ ID NO: 164 and SEQ ID NO: 189;
SEQ ID NO: 165 and SEQ ID NO: 190;
SEQ ID NO: 166 and SEQ ID NO: 191;
SEQ ID NO: 167 and SEQ ID NO: 192;
SEQ ID NO: 168 and SEQ ID NO: 193;
SEQ ID NO: 169 and SEQ ID NO: 194;
SEQ ID NO: 170 and SEQ ID NO: 195;
SEQ ID NO: 171 and SEQ ID NO: 196;
SEQ ID NO: 172 and SEQ ID NO: 197;
SEQ ID NO: 173 and SEQ ID NO: 198;
SEQ ID NO: 174 and SEQ ID NO: 199;
SEQ ID NO: 175 and SEQ ID NO: 200;
SEQ ID NO: 303 and SEQ ID NO: 320;
SEQ ID NO: 304 and SEQ ID NO: 321;
SEQ ID NO: 305 and SEQ ID NO: 322;
SEQ ID NO: 306 and SEQ ID NO: 323;
SEQ ID NO: 307 and SEQ ID NO: 324;
SEQ ID NO: 308 and SEQ ID NO: 325;
SEQ ID NO: 309 and SEQ ID NO: 326;
SEQ ID NO: 310 and SEQ ID NO: 327;
SEQ ID NO: 311 and SEQ ID NO: 328;
SEQ ID NO: 312 and SEQ ID NO: 329;
SEQ ID NO: 313 and SEQ ID NO: 330;
SEQ ID NO: 314 and SEQ ID NO: 331;
SEQ ID NO: 315 and SEQ ID NO: 332;
SEQ ID NO: 316 and SEQ ID NO: 333;
SEQ ID NO: 317 and SEQ ID NO: 334;
SEQ ID NO: 318 and SEQ ID NO: 335;
and
SEQ ID NO: 319 and SEQ ID NO: 336.

In some embodiments, anti-CD33 mAbs, antigen binding fragments thereof, and synthetic antigen-binding proteins such as scFvs comprise a combination of a V_{H} domain and a V_{L} domain, wherein the combination is chosen from those recited in Polypeptide No.s 1-25, e.g.:
SEQ ID NO: 151 and SEQ ID NO: 176;
SEQ ID NO: 152 and SEQ ID NO: 177;
SEQ ID NO: 153 and SEQ ID NO: 178;
SEQ ID NO: 154 and SEQ ID NO: 179;
SEQ ID NO: 155 and SEQ ID NO: 180;
SEQ ID NO: 156 and SEQ ID NO: 181;
SEQ ID NO: 157 and SEQ ID NO: 182;
SEQ ID NO: 158 and SEQ ID NO: 183;
SEQ ID NO: 159 and SEQ ID NO: 184;
SEQ ID NO: 160 and SEQ ID NO: 185;
SEQ ID NO: 161 and SEQ ID NO: 186;
SEQ ID NO: 162 and SEQ ID NO: 187;
SEQ ID NO: 163 and SEQ ID NO: 188;
SEQ ID NO: 164 and SEQ ID NO: 189;
SEQ ID NO: 165 and SEQ ID NO: 190;
SEQ ID NO: 166 and SEQ ID NO: 191;
SEQ ID NO: 167 and SEQ ID NO: 192;
SEQ ID NO: 168 and SEQ ID NO: 193;
SEQ ID NO: 169 and SEQ ID NO: 194;
SEQ ID NO: 170 and SEQ ID NO: 195;
SEQ ID NO: 171 and SEQ ID NO: 196;
SEQ ID NO: 172 and SEQ ID NO: 197;
SEQ ID NO: 173 and SEQ ID NO: 198;
SEQ ID NO: 174 and SEQ ID NO: 199;
and
SEQ ID NO: 175 and SEQ ID NO: 200.

In some embodiments, anti-CD33 mAbs, antigen binding fragments thereof, and synthetic antigen-binding proteins such as scFvs comprise a combination of a V_{H} domain and a V_{L} domain, wherein the combination is chosen from those recited in Polypeptide No.s 26-42, e.g.:
SEQ ID NO: 303 and SEQ ID NO: 320;
SEQ ID NO: 304 and SEQ ID NO: 321;
SEQ ID NO: 305 and SEQ ID NO: 322;
SEQ ID NO: 306 and SEQ ID NO: 323;
SEQ ID NO: 307 and SEQ ID NO: 324;
SEQ ID NO: 308 and SEQ ID NO: 325;
SEQ ID NO: 309 and SEQ ID NO: 326;
SEQ ID NO: 310 and SEQ ID NO: 327;
SEQ ID NO: 311 and SEQ ID NO: 328;
SEQ ID NO: 312 and SEQ ID NO: 329;
SEQ ID NO: 313 and SEQ ID NO: 330;
SEQ ID NO: 314 and SEQ ID NO: 331;
SEQ ID NO: 315 and SEQ ID NO: 332;
SEQ ID NO: 316 and SEQ ID NO: 333;
SEQ ID NO: 317 and SEQ ID NO: 334;
SEQ ID NO: 318 and SEQ ID NO: 335;
and
SEQ ID NO: 319 and SEQ ID NO: 336.

In some embodiments, anti-CD33 antibodies, antigen binding fragments thereof, and synthetic antigen-binding proteins such as scFvs may also comprise a combination of a variable heavy chain domain and a variable light chain domain wherein the variable heavy chain domain comprises a V_{H} sequence with at least 90% sequence identity, or at least 95%, 96% 97%, 98% or 99% sequence identity, to the variable heavy chain amino acid sequences shown above and/or wherein the variable light chain domain comprises a V_{L} sequence with at least 90% sequence identity, or at least 95%, 96% 97%, 98% or 99% sequence identity, to the variable light chain domain amino acid sequences shown above. The specific V_{H} and V_{L} pairings or combinations above may be preserved for anti-CD33 antibodies, antigen binding fragments thereof, and synthetic antigen-binding proteins such as scFvs having V_{H} and V_{L} domain sequences with a particular amino acid sequence percent identity to these reference sequences disclosed herein.

For all embodiments wherein the variable heavy chain and/or light chain domains of the antibodies, antigen binding fragments thereof, and synthetic antigen-binding proteins such as scFvs are defined by a particular amino acid sequence percent identity to a reference sequence, the V_{H} and/or V_{L} domains may retain identical CDR sequences to those present in the reference sequence such that the variation is present only within the framework regions.

### Anti-FLT3 Polypeptides

In some embodiments of the disclosure are provided anti-FLT3 polypeptides, including mAbs, antigen binding fragments thereof, and synthetic fusion proteins such as single-chain variable fragments (scFvs), comprising one or more complementarity-determining regions (CDRs) which recognize and bind FLT3. In some embodiments, the anti-FLT3 polypeptides, including mAbs, antigen binding fragments thereof, and synthetic fusion proteins such as single-chain variable fragments (scFvs) selectively bind a first polymorphic variant of FLT3 over a second polymorphic variant of FLT3; or selectively binds the second polymorphic variant of FLT3 over the first polymorphic variant. In some embodiments, the binding is at least 2-fold, 10-fold, or 30-fold selective.

In some embodiments, the first polymorphic variant of FLT3 is T227 and the second polymorphic variant of FLT3 is M227; or first polymorphic variant of FLT3 is M227 and the second polymorphic variant of FLT3 is T227.

Provided herein therefore, are a heavy chain variable (V_{H}) domain CDR1 (HCDR1), a V_{H} domain CDR2 (HCDR2), and a V_{H} domain CDR3 (HCDR3), and polypeptides comprising them.

Also provided is a V_{H} domain comprising one or more of these CDRs. The V_{H} domain of the anti-FLT3 mAb, antigen binding fragment thereof, or synthetic antigen-binding protein such as an scFv may comprise any one of the listed HCDR1 sequences in combination with any one of the HCDR2 sequences, and in combination with any one of the HCDR3 sequences. However, in certain embodiments, the provided HCDR1, HCDR2, and HCDR3 sequences are derived from a single common V_{H} domain, the examples of which are described herein.

The anti-FLT3 mAbs, antigen binding fragments thereof, and synthetic antigen-binding proteins such as scFvs may additionally comprise a light chain variable (V_{L}) domain, which is paired with the V_{H} domain to form an FLT3 antigen binding domain.

Provided herein therefore, are a light chain variable (V_{L}) domain CDR1 (LCDR1), a V_{L} domain CDR2 (LCDR2), and a V_{L} domain CDR3 (LCDR3), and polypeptides comprising them

Also provided is a V_{L} domain comprising one or more of these CDRs. The V_{L} domain of the anti-FLT3 mAb, antigen binding fragments thereof, or synthetic antigen-binding protein such as an scFv may comprise any one of the listed LCDR1 sequences in combination with any one of the LCDR2 sequences, and in combination with any one of the LCDR3 sequences. However, in certain embodiments, the LCDR1, LCDR2, and LCDR3 sequences are derived from a single common V_{L} domain, examples of which are described herein.

Also provided are mAbs, antigen binding fragments thereof, and synthetic antigen-binding proteins such as scFvs comprising the CDRs, V_{H} domains, and/or V_{L} domain disclosed herein. Any given anti-FLT3 mAb (and certain antigen-binding fragments thereof or scFv comprising a V_{H} domain paired with a V_{L} domain will comprise a combination of six (6) CDRs: a V_{H} domain CDR1 (HCDR1), a V_{H} domain CDR2 (HCDR2), and a V_{H} domain CDR3 (HCDR3), a V_{L} domain CDR1 (LCDR1), a V_{L} domain CDR2 (LCDR2), and a V_{L} domain CDR3 (LCDR3). Although all combinations of six (6) CDRs chosen from the CDR amino acid sequences described above are permissible and within the scope of the disclosure, certain combinations of the six (6) CDRs are provided herein.

Although all possible pairing of V_{H} domains and V_{L} domains chosen from the V_{H} and V_{L} domain amino acid sequences listed above are permissible and within the scope of the disclosure, some embodiments provide certain combinations of V_{H} and V_{L} domains.

In some embodiments, anti-FLT3 antibodies, antigen binding fragments thereof, and synthetic antigen-binding proteins such as scFvs may also comprise a combination of a variable heavy chain domain and a variable light chain domain wherein the variable heavy chain domain comprises a V_{H} sequence with at least 90% sequence identity, or at least 95%, 96% 97%, 98% or 99% sequence identity, to the variable heavy chain amino acid sequences shown above and/or wherein the variable light chain domain comprises a V_{L} sequence with at least 90% sequence identity, or at least 95%, 96% 97%, 98% or 99% sequence identity, to the variable light chain domain amino acid sequences shown above. The specific V_{H} and V_{L} pairings or combinations in parts (i) through may be preserved for anti-FLT3 antibodies having V_{H} and V_{L} domain sequences with a particular amino acid sequence percent identity to these reference sequences disclosed herein.

For all embodiments wherein the variable heavy chain and/or light chain domains of the antibodies, antigen binding fragments thereof, and synthetic antigen-binding proteins such as scFvs are defined by a particular amino acid sequence percent identity to a reference sequence, the V_{H} and/or V_{L} domains may retain identical CDR sequences to those present in the reference sequence such that the variation is present only within the framework regions.

### Anti-CLL-1 Polypeptides

In some embodiments of the disclosure are provided anti-CLL-1 polypeptides, including mAbs, antigen binding fragments thereof, and synthetic fusion proteins such as single-chain variable fragments (scFvs), comprising one or more complementarity-determining regions (CDRs) which recognize and bind CLL-1. In some embodiments, the anti-CLL-1 polypeptides, including mAbs, antigen binding fragments thereof, and synthetic fusion proteins such as single-chain variable fragments (scFvs) selectively bind a first polymorphic variant of CLL-1 over a second polymorphic variant of CLL-1; or selectively binds the second polymorphic variant of CLL-1 over the first polymorphic variant. In some embodiments, the binding is at least 2-fold, 10-fold, or 30-fold selective.

In some embodiments, the first polymorphic variant of CLL-1 is K224 and the second polymorphic variant of CLL-1 is Q244; or first polymorphic variant of CLL-1 is Q224 and the second polymorphic variant of CLL-1 is K244.

In some embodiments of the disclosure are provided anti-CLL-1 polypeptides, including mAbs, antigen binding fragments thereof, and synthetic fusion proteins such as single-chain variable fragments (scFvs), comprising one or more complementarity-determining regions (CDRs) which recognize and bind CLL-1. Sequences of the CDRs and V_{H} and V_{L} domains for the anti-CD33 polypeptides described herein for binding CLL-1 are provided in Tables and Examples below.

Provided herein therefore, are a heavy chain variable (V_{H}) domain CDR1 (HCDR1), a V_{H} domain CDR2 (HCDR2), and a V_{H} domain CDR3 (HCDR3), and polypeptides comprising them, wherein the HCDR1 comprises an amino acid sequence chosen from any of SEQ ID NOs 337-360 and 529-550; HCDR2 comprises an amino acid sequence chosen from any of SEQ ID NOs 361-384 and 551-572; and HCDR3 comprises an amino acid sequence chosen from any of SEQ ID NOs 385-408 and 573-594. Also provided are a HCDR1, a HCDR2, and a HCDR3, and polypeptides comprising them, wherein the HCDR1 comprises an amino acid sequence chosen from any of SEQ ID NOs 337-360; HCDR2 comprises an amino acid sequence chosen from any of SEQ ID NOs 361-384; and HCDR3 comprises an amino acid sequence chosen from any of SEQ ID NOs 361-384. Also provided are HCDR1, a HCDR2, and a HCDR3, and polypeptides comprising them, wherein the HCDR1 comprises an amino acid sequence chosen from any of SEQ ID NOs 529-550; HCDR2 comprises an amino acid sequence chosen from any of SEQ ID NOs 551-572; and HCDR3 comprises an amino acid sequence chosen from any of SEQ ID NOs 573-594.

Also provided is a V_{H} domain comprising one or more of these CDRs. The V_{H} domain of the anti-CLL-1 mAb, antigen binding fragment thereof, or synthetic antigen-binding protein such as an scFv may comprise any one of the listed HCDR1 sequences in combination with any one of the HCDR2 sequences, and in combination with any one of the HCDR3 sequences. However, in certain embodiments, the provided HCDR1, HCDR2, and HCDR3 sequences are derived from a single common V_{H} domain, the examples of which are described herein.

The anti-CLL-1 mAbs, antigen binding fragments thereof, and synthetic antigen-binding proteins such as scFvs may additionally comprise a light chain variable (V_{L}) domain, which is paired with the V_{H} domain to form an CLL-1 antigen binding domain.

Provided herein therefore, are a light chain variable (V_{L}) domain CDR1 (LCDR1), a V_{L} domain CDR2 (LCDR2), and a V_{L} domain CDR3 (LCDR3), and polypeptides comprising them, wherein the LCDR1 comprises an amino acid sequence chosen from: SEQ ID NOs 409-432 and 595-616; LCDR2 comprises an amino acid sequence chosen from: SEQ ID NOs 433-456 and 617-638; and LCDR3 comprises an amino acid sequence chosen from: SEQ ID NOs 457-480 and 639-660. Also provided are a LCDR1, a LCDR2, and a LCDR3, and polypeptides comprising them, wherein the LCDR1 comprises an amino acid sequence chosen from any of SEQ ID NOs 409-432; LCDR2 comprises an amino acid sequence chosen from any of SEQ ID NOs 433-456; and LCDR3 comprises an amino acid sequence chosen from any of SEQ ID NOs 433-456. Also provided are a LCDR1, a LCDR2, and a LCDR3, and polypeptides comprising them, wherein the LCDR1 comprises an amino acid sequence chosen from any of SEQ ID NOs 595-616; LCDR2 comprises an amino acid sequence chosen from any of SEQ ID NOs 617-638; and LCDR3 comprises an amino acid sequence chosen from any of SEQ ID NOs 639-660.

Also provided is a V_{L} domain comprising one or more of these CDRs. The V_{L} domain of the anti-CLL-1 mAb, antigen binding fragments thereof, or synthetic antigen-binding protein such as an scFv may comprise any one of the listed LCDR1 sequences in combination with any one of the LCDR2 sequences, and in combination with any one of the LCDR3 sequences. However, in certain embodiments, the LCDR1, LCDR2, and LCDR3 sequences are derived from a single common V_{L} domain, examples of which are described herein.

Also provided are mAbs, antigen binding fragments thereof, and synthetic antigen-binding proteins such as scFvs comprising the CDRs, V_{H} domains, and/or V_{L} domain disclosed herein. Any given anti-CLL-1 mAb (and certain antigen-binding fragments thereof or scFv comprising a V_{H} domain paired with a V_{L} domain will comprise a combination of six (6) CDRs: a V_{H} domain CDR1 (HCDR1), a V_{H} domain CDR2 (HCDR2), and a V_{H} domain CDR3 (HCDR3), a V_{L} domain CDR1 (LCDR1), a V_{L} domain CDR2 (LCDR2), and a V_{L} domain CDR3 (LCDR3). Although all combinations of six (6) CDRs chosen from the CDR amino acid sequences described above are permissible and within the scope of the disclosure, certain combinations of the six (6) CDRs are provided herein.

In some embodiments, the combination of the six (6) CDRs is chosen from the combinations recited in each of Polypeptide No.s 43-88. In some embodiments, the combination of the six (6) CDRs is chosen from the combinations recited in each of Polypeptide No.s 43-66. In some embodiments, the combination of the six (6) CDRs is chosen from the combinations recited in each of Polypeptide No.s 67-88.

In some embodiments, the anti-CLL-1 mAbs, antigen binding fragments thereof, and synthetic antigen-binding proteins such as scFvs comprise a V_{H} domain chosen from any of SEQ ID NOs 481-504 and 661-682, with amino acid sequences exhibiting at least 90% sequence identity, or at least 95%, 96% 97%, 98% or 99% sequence identity to one of the recited amino acid sequences. In some embodiments, the anti-CD33 mAbs, antigen binding fragments thereof, and synthetic antigen-binding proteins such as scFvs comprise a V_{H} domain chosen from any of SEQ ID NOs 481-504, with amino acid sequences exhibiting at least 90% sequence identity, or at least 95%, 96% 97%, 98% or 99% sequence identity to one of the recited amino acid sequences. In some embodiments, the anti-CD33 mAbs, antigen binding fragments thereof, and synthetic antigen-binding proteins such as scFvs comprise a V_{H} domain chosen from any of SEQ ID NOs 661-682, with amino acid sequences exhibiting at least 90% sequence identity, or at least 95%, 96% 97%, 98% or 99% sequence identity to one of the recited amino acid sequences.

Alternatively, or in addition, the anti-CD33 mAbs, antigen binding fragments thereof, and synthetic antigen-binding proteins such as scFvs comprise a V_{L} domain having an amino acid sequence chosen from any of SEQ ID NOs 505-528 and 683-704, and amino acid sequences exhibiting at least 90% sequence identity, or at least 95%, 96% 97%, 98% or 99% sequence identity to one of the recited amino acid sequences. In some embodiments, the anti-CD33 mAbs, antigen binding fragments thereof, and synthetic antigen-binding proteins such as scFvs comprise a V_{L} domain having an amino acid sequence chosen from any of SEQ ID NOs 505-528, and amino acid sequences exhibiting at least 90% sequence identity, or at least 95%, 96% 97%, 98% or 99% sequence identity to one of the recited amino acid sequences. In some embodiments, the anti-CD33 mAbs, antigen binding fragments thereof, and synthetic antigen-binding proteins such as scFvs comprise a V_{L} domain having an amino acid sequence chosen from any of SEQ ID NOs 683-704, and amino acid sequences exhibiting at least 90% sequence identity, or at least 95%, 96% 97%, 98% or 99% sequence identity to one of the recited amino acid sequences.

Although all possible pairing of V_{H} domains and V_{L} domains chosen from the V_{H} and V_{L} domain amino acid sequences listed above are permissible and within the scope of the disclosure, some embodiments provide certain combinations of V_{H} and V_{L} domains. Accordingly, in some embodiments, anti-CD33 mAbs, antigen binding fragments thereof, and synthetic antigen-binding proteins such as scFvs comprise a combination of a V_{H} domain and a V_{L} domain, wherein the combination is chosen from those recited in Polypeptide No.s 43-88, e.g.:
SEQ ID NO: 481 and SEQ ID NO: 505;
SEQ ID NO: 482 and SEQ ID NO: 506;
SEQ ID NO: 483 and SEQ ID NO: 507;
SEQ ID NO: 484 and SEQ ID NO: 508;
SEQ ID NO: 485 and SEQ ID NO: 509;
SEQ ID NO: 486 and SEQ ID NO: 510;
SEQ ID NO: 487 and SEQ ID NO: 511;
SEQ ID NO: 488 and SEQ ID NO: 512;
SEQ ID NO: 489 and SEQ ID NO: 513;
SEQ ID NO: 490 and SEQ ID NO: 514;
SEQ ID NO: 491 and SEQ ID NO: 515;
SEQ ID NO: 492 and SEQ ID NO: 516;
SEQ ID NO: 493 and SEQ ID NO: 517;
SEQ ID NO: 494 and SEQ ID NO: 518;
SEQ ID NO: 495 and SEQ ID NO: 519;
SEQ ID NO: 496 and SEQ ID NO: 520;
SEQ ID NO: 497 and SEQ ID NO: 521;
SEQ ID NO: 498 and SEQ ID NO: 522;
SEQ ID NO: 499 and SEQ ID NO: 523;
SEQ ID NO: 500 and SEQ ID NO: 524;
SEQ ID NO: 501 and SEQ ID NO: 525;
SEQ ID NO: 502 and SEQ ID NO: 526;
SEQ ID NO: 503 and SEQ ID NO: 527;
SEQ ID NO: 504 and SEQ ID NO: 528;
SEQ ID NO: 661 and SEQ ID NO: 683;
SEQ ID NO: 662 and SEQ ID NO: 684;
SEQ ID NO: 663 and SEQ ID NO: 685;
SEQ ID NO: 664 and SEQ ID NO: 686;
SEQ ID NO: 665 and SEQ ID NO: 687;
SEQ ID NO: 666 and SEQ ID NO: 688;
SEQ ID NO: 667 and SEQ ID NO: 689;
SEQ ID NO: 668 and SEQ ID NO: 690;
SEQ ID NO: 669 and SEQ ID NO: 691;
SEQ ID NO: 670 and SEQ ID NO: 692;
SEQ ID NO: 671 and SEQ ID NO: 693;
SEQ ID NO: 672 and SEQ ID NO: 694;
SEQ ID NO: 673 and SEQ ID NO: 695;
SEQ ID NO: 674 and SEQ ID NO: 696;
SEQ ID NO: 675 and SEQ ID NO: 697;
SEQ ID NO: 676 and SEQ ID NO: 698;
SEQ ID NO: 677 and SEQ ID NO: 699;
SEQ ID NO: 678 and SEQ ID NO: 700;
SEQ ID NO: 679 and SEQ ID NO: 701;
SEQ ID NO: 680 and SEQ ID NO: 702;
SEQ ID NO: 681 and SEQ ID NO: 703;
and
SEQ ID NO: 682 and SEQ ID NO: 704.

In some embodiments, anti-CD33 mAbs, antigen binding fragments thereof, and synthetic antigen-binding proteins such as scFvs comprise a combination of a V_{H} domain and a V_{L} domain, wherein the combination is chosen from those recited in Polypeptide No.s 43-66, e.g.:
SEQ ID NO: 481 and SEQ ID NO: 505;
SEQ ID NO: 482 and SEQ ID NO: 506;
SEQ ID NO: 483 and SEQ ID NO: 507;
SEQ ID NO: 484 and SEQ ID NO: 508;
SEQ ID NO: 485 and SEQ ID NO: 509;
SEQ ID NO: 486 and SEQ ID NO: 510;
SEQ ID NO: 487 and SEQ ID NO: 511;
SEQ ID NO: 488 and SEQ ID NO: 512;
SEQ ID NO: 489 and SEQ ID NO: 513;
SEQ ID NO: 490 and SEQ ID NO: 514;
SEQ ID NO: 491 and SEQ ID NO: 515;
SEQ ID NO: 492 and SEQ ID NO: 516;
SEQ ID NO: 493 and SEQ ID NO: 517;
SEQ ID NO: 494 and SEQ ID NO: 518;
SEQ ID NO: 495 and SEQ ID NO: 519;
SEQ ID NO: 496 and SEQ ID NO: 520;
SEQ ID NO: 497 and SEQ ID NO: 521;
SEQ ID NO: 498 and SEQ ID NO: 522;
SEQ ID NO: 499 and SEQ ID NO: 523;
SEQ ID NO: 500 and SEQ ID NO: 524;
SEQ ID NO: 501 and SEQ ID NO: 525;
SEQ ID NO: 502 and SEQ ID NO: 526;
SEQ ID NO: 503 and SEQ ID NO: 527;
and
SEQ ID NO: 504 and SEQ ID NO: 528.

In some embodiments, anti-CD33 mAbs, antigen binding fragments thereof, and synthetic antigen-binding proteins such as scFvs comprise a combination of a V_{H} domain and a V_{L} domain, wherein the combination is chosen from those recited in Polypeptide No.s 67-88, e.g.:
SEQ ID NO: 661 and SEQ ID NO: 683;
SEQ ID NO: 662 and SEQ ID NO: 684;
SEQ ID NO: 663 and SEQ ID NO: 685;
SEQ ID NO: 664 and SEQ ID NO: 686;
SEQ ID NO: 665 and SEQ ID NO: 687;
SEQ ID NO: 666 and SEQ ID NO: 688;
SEQ ID NO: 667 and SEQ ID NO: 689;
SEQ ID NO: 668 and SEQ ID NO: 690;
SEQ ID NO: 669 and SEQ ID NO: 691;
SEQ ID NO: 670 and SEQ ID NO: 692;
SEQ ID NO: 671 and SEQ ID NO: 693;
SEQ ID NO: 672 and SEQ ID NO: 694;
SEQ ID NO: 673 and SEQ ID NO: 695;
SEQ ID NO: 674 and SEQ ID NO: 696;
SEQ ID NO: 675 and SEQ ID NO: 697;
SEQ ID NO: 676 and SEQ ID NO: 698;
SEQ ID NO: 677 and SEQ ID NO: 699;
SEQ ID NO: 678 and SEQ ID NO: 700;
SEQ ID NO: 679 and SEQ ID NO: 701;
SEQ ID NO: 680 and SEQ ID NO: 702;
SEQ ID NO: 681 and SEQ ID NO: 703;
and
SEQ ID NO: 682 and SEQ ID NO: 704.

In some embodiments, anti-CLL-1 antibodies, antigen binding fragments thereof, and synthetic antigen-binding proteins such as scFvs may also comprise a combination of a variable heavy chain domain and a variable light chain domain wherein the variable heavy chain domain comprises a V_{H} sequence with at least 90% sequence identity, or at least 95%, 96% 97%, 98% or 99% sequence identity, to the variable heavy chain amino acid sequences shown above and/or wherein the variable light chain domain comprises a V_{L} sequence with at least 90% sequence identity, or at least 95%, 96% 97%, 98% or 99% sequence identity, to the variable light chain domain amino acid sequences shown above. The specific V_{H} and V_{L} pairings or combinations in parts (i) through may be preserved for anti-CLL-1 antibodies having V_{H} and V_{L} domain sequences with a particular amino acid sequence percent identity to these reference sequences disclosed herein.

For all embodiments wherein the variable heavy chain and/or light chain domains of the antibodies, antigen binding fragments thereof, and synthetic antigen-binding proteins such as scFvs are defined by a particular amino acid sequence percent identity to a reference sequence, the V_{H} and/or V_{L} domains may retain identical CDR sequences to those present in the reference sequence such that the variation is present only within the framework regions.

### Chimeric Antigen Receptors (CARs) and CAR-Bearing Immune Effector Cells

Also provided herein are chimeric antigen receptors (CARs; and transgenic T-cell receptors, TCRs) comprising polypeptides as disclosed herein, e.g. as disclosed in Tables 2, 3, 12 and 13, and immune effector cells expressing them. A CAR is a recombinant fusion protein comprising: 1) an extracellular ligand-binding domain, i.e., an antigen-recognition domain, 2) a hinge domain, 3) a transmembrane domain, and 4) a cytoplasmic signaling domain, 5) and optionally, a co-stimulatory domain.

Methods for CAR design, delivery and expression, and the manufacturing of clinical-grade CAR-T cell populations are known in the art. CAR designs are generally tailored to each cell type.

The extracellular ligand-binding domain of a chimeric antigen receptor recognizes and specifically binds an antigen, typically a surface-expressed antigen of a malignant cell. The extracellular ligand-binding domain specifically binds an antigen when, for example, it binds the antigen with an affinity constant or affinity of interaction (K_{D}) between about 0.1 pM to about 10 μM, or about 0.1 pM to about 1 μM, or about 0.1 pM to about 100 nM. Methods for determining the affinity of interaction are known in the art. An extracellular ligand-binding domain can also be said to specifically bind a first polymorphic variant of an antigen when it binds it selectively over a second polymorphic variant of the same antigen.

An extracellular ligand-binding domain suitable for use in a CAR may be any antigen-binding polypeptide, a wide variety of which are known in the art. In some instances, the extracellular ligand-binding domain is a single chain Fv (scFv). Other antibody based recognition domains (cAb VHH (camelid antibody variable domains) and humanized versions thereof, lgNAR V_{H} (shark antibody variable domains) and humanized versions thereof, sdAb V_{H} (single domain antibody variable domains) and “camelized” antibody variable domains are suitable for use. In some instances, T-cell receptor (TCR) based recognition domains such as single chain TCR (scTv, single chain two-domain TCR containing VαVβ) are also suitable for use. In some embodiments, the extracellular ligand-binding domain is constructed from a natural binding partner, or a functional fragment thereof, to a target antigen. For example, CARs in general may be constructed with a portion of the APRIL protein, targeting the ligand for the B-Cell Maturation Antigen (BCMA) and Transmembrane Activator and CAML Interactor (TACI), effectively co-targeting both BCMA and TACI for the treatment of multiple myeloma.

The targeted antigen to which the CAR binds via its extracellular ligand-binding domain may be an antigen that is expressed on a malignant myeloid (AML) cell, T cell or other cell. Antigens expressed on a malignant myeloid (AML) cells include CD33, FLT3, CD123, and CLL-1. Antigens expressed on T cells include CD2, CD3, CD4, CD5, CD7, TCRα (TRAC), and TCRβ. Antigens expressed on malignant plasma cells include BCMA, CS1, CD38, CD79A, CD79B, CD138, and CD19. Antigens expressed on malignant B cells include CD19, CD20, CD21, CD22, CD23, CD24, CD25, CD27, CD38, and CD45.

Typically, the extracellular ligand-binding domain is linked to the intracellular domain of the chimeric antigen receptor by a transmembrane (TM) domain. A peptide hinge connects the extracellular ligand-binding domain to the transmembrane domain. A transmembrane domain traverses the cell membrane, anchors the CAR to the T cell surface, and connects the extracellular ligand-binding to the cytoplasmic signaling domain, thus impacting expression of the CAR on the T cell surface.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMFI, CD150, IPO-3), BLAME (SLAMF8), SELPLG (CD162), LTBR, and PAG/Cbp. Alternatively, the transmembrane domain can be synthetic and comprise predominantly hydrophobic amino acid residues (e.g., leucine and valine). In some cases, a triplet of phenylalanine, tryptophan and valine will be found at each end of a synthetic transmembrane domain. In some embodiments, the transmembrane domain is derived from the T-cell surface glycoprotein CD8 alpha chain isoform 1 precursor (NP_001139345.1) or CD28. A short oligo- or polypeptide linker, such as between 2 and 10 amino acids in length, may form the linkage between the transmembrane domain and the endoplasmic domain of the CAR. In some embodiments, the CAR has more than one transmembrane domain, which can be a repeat of the same transmembrane domain, or can be different transmembrane domains.

NK cells express a number of transmembrane (TM) adapters that signal activation, that are triggered via association with activating receptors. This provides an NK cell specific signal enhancement via engineering the TM domains from activating receptors, and thereby harness endogenous adapters. The TM adapter can be any endogenous TM adapter capable of signaling activation. In some embodiments, the TM adapter may be chosen from FceR1γ (ITAMx1), CD3ζ (ITAMx3), DAP12 (ITAMx1), or DAP10 (YxxM/YINM), NKG2D, FcγRIIIa, NKp44, NKp30, NKp46, actKIR, NKG2C, CD8α, and IL15Rb.

The CAR can further comprise a hinge region between extracellular ligand-binding domain and said transmembrane domain. The term “hinge region” (equivalently, “hinge” or “spacer”) generally means any oligo- or polypeptide that functions to link the transmembrane domain to the extracellular ligand-binding domain. In particular, hinge region is used to provide more flexibility and accessibility for the extracellular ligand binding domain, and can confer stability for efficient CAR expression and activity. A hinge region may comprise up to 300 amino acids, preferably 10 to 100 amino acids and most preferably 25 to 50 amino acids. Hinge region may be derived from all or parts of naturally-occurring molecules such as CD28, 4-1BB (CD137), OX-40 (CD134), CD3ζ, the T cell receptor α or β chain, CD45, CD4, CD5, CD8, CD8a, CD9, CD16, CD22, CD33, CD37, CD64, CD80, CD86, ICOS, CD154 or from all or parts of an antibody constant region. In some embodiments, for example, the hinge sequence is derived from a CD8a molecule or a CD28 molecule. Alternatively, the hinge region may be a synthetic sequence that corresponds to a naturally-occurring hinge sequence or the hinge region may be an entirely synthetic hinge sequence. In one embodiment, the hinge domain comprises a part of human CD8a, FcγRIIIα receptor, or IgGI, and have at least 80%, 90%, 95%, 97%, or 99% sequence identity thereto.

After antigen recognition, the cytoplasmic signaling domain transmits a signal to the immune effector cell, activating at least one of the normal effector functions of the immune effector cell. Effector function of a T cell, for example, may be cytolytic activity or helper activity including the secretion of cytokines. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3δ, CD32 (Fc gamma RIIa), DAP10, DAP12, CD79a, CD79b, FcγRIγ, FcγRIIIγ, FcεRIβ (FCERIB), and FcεRIγ (FCERIG).

First-generation CARs typically have the cytoplasmic signaling domain from the CD3 chain, which is the primary transmitter of signals from endogenous TCRs. Second-generation CARs add cytoplasmic signaling domains from various co-stimulatory protein receptors (e.g., CD28, 4-1BB, ICOS) to the cytoplasmic signaling domain of the CAR to provide additional signals to the T cell.

A “costimulatory domain” is derived from the intracellular signaling domains of costimulatory proteins that enhance cytokine production, proliferation, cytotoxicity, and/or persistence in vivo. Preclinical studies have indicated that the second generation of CAR designs improves the antitumor activity of T cells. More recent, third-generation, and later generation, CARs combine multiple costimulatory domains to further augment potency. T cells grafted with these CARs have demonstrated improved expansion, activation, persistence, and tumor-eradicating efficiency independent of costimulatory receptor/ligand interaction.

For example, the cytoplasmic signaling domain of the CAR can be designed to comprise the signaling domain (e.g., CD3ζ) by itself or combined with any other desired cytoplasmic domain(s) useful in the context of the CAR. For example, the cytoplasmic domain of the CAR can comprise a signaling domain (e.g., CD3ζ) chain portion and a costimulatory signaling region. The co-stimulatory signaling region refers to a portion of the CAR comprising the intracellular domain of a co-stimulatory molecule. Examples of such molecules include CD27, CD28, 4-1BB (CD137), OX40, CD30, CD40, ICOS, LFA-1, CD2, CD7, LIGHT, NKG2C, B7-H3, and a ligand that specifically binds with CD83, CD8, CD4, b2c, CD80, CD86, DAP10, DAP12, MyD88, BTNL3, and NKG2D.

In some embodiments, the cytoplasmic signaling domain is a CD3 zeta (CD3ζ) signaling domain. In some embodiments, the co-stimulatory domain comprises the cytoplasmic domain of CD28, 4-1BB, or a combination thereof. In some cases, the co-stimulatory signaling region contains 1, 2, 3, or 4 cytoplasmic domains of one or more intracellular signaling and/or co-stimulatory molecules.

The co-stimulatory signaling domain(s) may contain one or more mutations in the cytoplasmic domains of CD28 and/or 4-1BB that enhance signaling. In some embodiments, the disclosed CARs comprises a co-stimulatory signaling region comprising a mutated form of the cytoplasmic domain of CD28 with altered phosphorylation at Y206 and/or Y218. In some embodiments, the disclosed CAR comprises an attenuating mutation at Y206, which will reduce the activity of the CAR. In some embodiments, the disclosed CAR comprises an attenuating mutation at Y218, which will reduce expression of the CAR. Any amino acid residue, such as alanine or phenylalanine, can be substituted for the tyrosine to achieve attenuation. In some embodiments, the tyrosine at Y206 and/or Y218 is substituted with a phosphomimetic residue. In some embodiments, the disclosed CAR substitution of Y206 with a phosphomimetic residue, which will increase the activity of the CAR. In some embodiments, the disclosed CAR comprises substitution of Y218 with a phosphomimetic residue, which will increase expression of the CAR. For example, the phosphomimetic residue can be phosphotyrosine. In some embodiments, a CAR may contain a combination of phosphomimetic amino acids and substitution(s) with non-phosphorylatable amino acids in different residues of the same CAR. For instance, a CAR may contain an alanine or phenylalanine substitution in Y209 and/or Y191 plus a phosphomimetic substitution in Y206 and/or Y218.

In some embodiments, the disclosed CARs comprises one or more 4-1BB domains with mutations that enhance binding to specific TRAF proteins, such as TRAF1, TRAF2, TRAF3, TRAF4, TRAF5, TRAF6, or any combination thereof. In some cases, the 41BB mutation enhances TRAF1- and/or TRAF2-dependent proliferation and survival of the T-cell, e.g. through NF-kB. In some cases, the 4-1BB mutation enhances TRAF3-dependent antitumor efficacy, e.g. through IRF7/INFβ. Therefore, the disclosed CARs can comprise cytoplasmic domain(s) of 4-1BB having at least one mutation in these sequences that enhance TRAF-binding and/or enhance NFκB signaling.

Also as disclosed herein, TRAF proteins can in some cases enhance CAR T cell function independent of NFκB and 4-1BB. For example, TRAF proteins can in some cases enhance CD28 co-stimulation in T cells. Therefore, also disclosed herein are immune effector cells co-expressing CARs with one or more TRAF proteins, such as TRAF1, TRAF2, TRAF3, TRAF4, TRAF5, TRAF6, or any combination thereof. In some cases, the CAR is any CAR that targets a tumor antigen. For example, first-generation CARs typically had the intracellular domain from the CD3 chain, while second-generation CARs added intracellular signaling domains from various costimulatory protein receptors (e.g., CD28, 4-1BB, ICOS) to the cytoplasmic signaling domain of the CAR to provide additional signals to the T cell. In some cases, the CAR is the disclosed CAR with enhanced 4-1BB activation.

Variations on CAR components may be advantageous, depending upon the type of cell in which the CAR is expressed.

For example, in NK cells, in some embodiments, the transmembrane domain can be a sequence associated with NKG2D, FcγRIIIa, NKp44, NKp30, NKp46, actKIR, NKG2C, or CD8α. In certain embodiments, the NK cell is a ML-NK or CIML-NK cell and the TM domain is CD8α. Certain™ domains that do not work well in NK cells generally may work in a subset; CD8α, for example, works in ML-NKs but not NK cells generally.

Similarly, in NK cells, in some embodiments, the intracellular signaling domain(s) can be any co-activating receptor(s) capable of functioning in an NK cell, such as, for example, CD28, CD137/41BB (TRAF, NFkB), CD134/OX40, CD278/ICOS, DNAM-1 (Y-motif), NKp80 (Y-motif), 2B4 (SLAMF)::ITSM, CRACC (CS1/SLAMF7)::ITSM, CD2 (Y-motifs, MAPK/Erk), CD27 (TRAF, NFkB), or integrins (e.g., multiple integrins).

Similarly, in NK cells, in some embodiments, an intracellular signaling domain can be a cytokine receptor capable of functioning in an NK cell. For example, a cytokine receptor can be a cytokine receptor associated with persistence, survival, or metabolism, such as IL-2/15Rbyc::Jak1/3, STAT3/5, PI3K/mTOR, MAPK/ERK. As another example, a cytokine receptor can be a cytokine receptor associated with activation, such as IL-18R::NFkB. As another example, a cytokine receptor can be a cytokine receptor associated with IFN-γ production, such as IL-12R::STAT4. As another example, a cytokine receptor can be a cytokine receptor associated with cytotoxicity or persistence, such as IL-21R::Jak3/Tyk2, or STAT3. As another example, an intracellular signaling domain can be a TM adapter, such as FceR1γ (ITAMx1), CD3ζ (ITAMx3), DAP12 (ITAMx1), or DAP10 (YxxM/YINM). As another example, CAR intracellular signaling domains (also known as endodomains) can be derived from costimulatory molecules from the CD28 family (such as CD28 and ICOS) or the tumor necrosis factor receptor (TNFR) family of genes (such as 4-1BB, OX40, or CD27). The TNFR family members signal through recruitment of TRAF proteins and are associated with cellular activation, differentiation and survival. Certain signaling domains that may not work well in all NK cells generally may work in a subset; CD28 or 4-1BB, for example, work in ML-NKs.

Methods of Making CARs and CAR-Bearing Cells

The chimeric antigen receptor (CAR) construct, which encodes the chimeric receptor can be prepared in conventional ways. Since, for the most part, natural sequences are employed, the natural genes are isolated and manipulated, as appropriate (e.g., when employing a Type II receptor, the immune signaling receptor component may have to be inverted), so as to allow for the proper joining of the various components. Thus, the nucleic acid sequences encoding for the N-terminal and C-terminal proteins of the chimeric receptor can be isolated by employing the polymerase chain reaction (PCR), using appropriate primers which result in deletion of the undesired portions of the gene. Alternatively, restriction digests of cloned genes can be used to generate the chimeric construct. In either case, the sequences can be selected to provide for restriction sites which are blunt-ended, or have complementary overlaps.

The various manipulations for preparing the chimeric construct can be carried out in vitro and in particular embodiments the chimeric construct is introduced into vectors for cloning and expression in an appropriate host using standard transformation or transfection methods. Thus, after each manipulation, the resulting construct from joining of the DNA sequences is cloned, the vector isolated, and the sequence screened to ensure that the sequence encodes the desired chimeric receptor. The sequence can be screened by restriction analysis, sequencing, or the like.

A chimeric construct can be introduced into immune effector cells as naked DNA or in a suitable vector. Methods of stably transfecting immune effector cells by electroporation using naked DNA are known in the art. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cell, e.g., T cells. Suitable vectors are non-replicating in the immune effector cells of the subject. A large number of vectors are known which are based on viruses, where the copy number of the virus maintained in the cell is low enough to maintain the viability of the cell. Illustrative vectors include the pFB-neo vectors (STRATAGENE™) as well as vectors based on HIV, SV40, EBV, HSV or BPV. Once it is established that the transfected or transduced immune effector cell is capable of expressing the chimeric receptor as a surface membrane protein with the desired regulation and at a desired level, it can be determined whether the chimeric receptor is functional in the host cell to provide for the desired signal induction (e.g., production of Rantes, Mip1-alpha, GM-CSF upon stimulation with the appropriate ligand).

Engineered CARs may be introduced into CAR-bearing immune effector cells using retroviruses, which efficiently and stably integrate a nucleic acid sequence encoding the chimeric antigen receptor into the target cell genome. Other methods known in the art include, but are not limited to, lentiviral transduction, transposon-based systems, direct RNA transfection, and CRISPR/Cas systems (e.g., type I, type II, or type III systems using a suitable Cas protein such Cas3, Cas4, Cas5, Cas5e (or CasD), Cash, Cas6e, Cas6f, Cas7, Cas8a1, Cas8a2, Cas8b, Cas8c, Cas9, Cas10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966, etc.). Zinc finger nucleases (ZFNs) and transcription activator-like effector nucleases (TALENs) may also be used. See, e.g., Shearer R F and Saunders D N, “Experimental design for stable genetic manipulation in mammalian cell lines: lentivirus and alternatives,” Genes Cells 2015 January; 20(1):1-10.

Amino acid sequences for selected components which may be used to construct a CAR are disclosed below in Table 1.

TABLE 1
Amino acid sequences of selected CAR components.

| Functional domains | SEQ ID NO: | Amino acid sequence |
|---|---|---|
| CD8α signal peptide | 1521 | MALPVTALLLPLALLLHAARP |
| (variant 1) | | |
| CD8α signal peptide | 1522 | MALPVTALLLPLALLLHAA |
| (variant 2) | | |
| CD8α signal peptide | 1523 | MALPVTALLLP |
| (variant 3) | | |
| CD8α signal peptide | 1524 | PVTALLLPLALL |
| (variant 4) | | |
| CD8α signal peptide | 1525 | LLLPLALLLHAARP |
| (variant 5) | | |
| CD8α hinge | 1526 | TTTPAPRPPTPAPTIASQPLSLRPEACRPA |
| | | AGGAVHTRGLDFACD |
| CD28 | 1527 | FWVLVVVGGVLACYSLLVTVAFIIFWV |
| Transmembrane | | |
| (Tₘ) domain | | |
| Surface glycoprotein | 1528 | MALPVTALLLPLALLLHAARPSQFRVSP |
| CD8 alpha chain | | LDRTWNLGETVELKCQVLLSNPTSGCS |
| isoform 1 precursor | | WLFQPRGAAASPTFLLYLSQNKPKAAEG |
| (NP_001139345.1) | | LDTQRFSGKRLGDTFVLTLSDFRRENEG |
| | | YYFCSALSNSIMYFSHFVPVFLPAKPTTT |
| | | PAPRPPTPAPTIASQPLSLRPEACRPAAG |
| | | GAVHTRGLDFACDIYIWAPLAGTCGVLL |
| | | LSLVITLYCNHRNRRRVCKCPRPVVKSG |
| | | DKPSLSARYV |
| 4-1BB costimulatory | 1529 | KRGRKKLLYIFKQPFMRPVQTTQEEDGC |
| domain | | SCRFPEEEEGGCEL |
| CD28 costimulatory | 1530 | RSKRSRLLHSDYMNMTPRRPGPTRKHY |
| domain | | QPYAPPRDFAAYRS |
| CD3 zeta (ζ) | 1531 | RVKFSRSADAPAYKQGQNQLYNELNLG |
| | | RREEYDVLDKRRGRDPEMGGKPRRKNP |
| | | QEGLYNELQKDKMAEAYSEIGMKGERR |
| | | RGKGHDGLYQGLSTATKDTYDALHMQ |
| | | ALPPR |
| P2A peptide | 1532 | GSGATNFSLLKQAGDVEENPGP |
| (GGGGS) linker | 1533 | GGGGS |
| (GGGGS)₂ linker | 1534 | GGGGSGGGGS |
| (GGGGS)₃ linker | 1535 | GGGGSGGGGSGGGGS |
| (GGGGS)₄ linker | 1536 | GGGGSGGGGSGGGGSGGGGS |
| hCD34 | 1537 | MPRGWTALCLLSLLPSGFMSLDNNGTA |
| | | TPELPTQGTFSNVSTNVSYQETTTPSTLG |
| | | STSLHPVSQHGNEATTNITETTVKFTSTS |
| | | VITSVYGNTNSSVQSQTSVISTVFTTPAN |
| | | VSTPETTLKPSLSPGNVSDLSTTSTSLATS |
| | | PTKPYTSSSPILSDIKAEIKCSGIREVKLT |
| | | QGICLEQNKTSSCAEFKKDRGEGLARVL |
| | | CGEEQADADAGAQVCSLLLAQSEVRPQ |
| | | CLLLVLANRTEISSKLQLMKKHQSDLKK |
| | | LGILDFTEQDVASHQSYSQKTLIALVTSG |
| | | ALLAVLGITGYFLMNRRSWSPI |
| Human-Herpes | 1538 | MPRGWTALCLLSLLPSGFMSLDNNGTA |
| Simplex Virus-1 | | TPELPTQGTFSNVSTNVSYQETTTPSTLG |
| (HSV) - thymidine | | STSLHPVSQHGNEATTNITETTVKFTSTS |
| kinase (TK) | | VITSVYGNTNSSVQSQTSVISTVFTTPAN |
| | | VSTPETTLKPSLSPGNVSDLSTTSTSLATS |
| | | PTKPYTSSSPILSDIKAEIKCSGIREVKLT |
| | | QGICLEQNKTSSCAEFKKDRGEGLARVL |
| | | CGEEQADADAGAQVCSLLLAQSEVRPQ |
| | | CLLLVLANRTEISSKLQLMKKHQSDLKK |
| | | LGILDFTEQDVASHQSYSQKTLIALVTSG |
| | | ALLAVLGITGYFLMNRRSWSPTGEGGG |
| | | GGDLGGVKLPHLFGKRLVEARMASYPC |
| | | HQHASAFDQAARSRGHSNRRTALRPRR |
| | | QQEATEVRLEQKMPTLLRVYIDGPHGM |
| | | GKTTTTQLLVALGSRDDIVYVPEPMTY |
| | | WQVLGASETIANIYTTQHRLDQGEISAG |
| | | DAAVVMTSAQITMGMPYAVTDAVLAP |
| | | HVGGEAGSSHAPPPALTLLLDRHPIAVM |
| | | LCYPAARYLMGSMTPQAVLAFVALIPPT |
| | | LPGTNIVLGALPEDRHIDRLAKRQRPGE |
| | | RLDLAMLAAIRRVYGLLANTVRYLQGG |
| | | GSWWEDWGQLSGTAVPPQGAEPQSNA |
| | | GPRPHIGDTLFTLFRAPELLAPNGDLYNV |
| | | FAWALDVLAKRLRPMHVFILDYDQSPA |
| | | GCRDALLQLTSGMVQTHVTTPGSIPTIC |
| | | DLARTFAREMGEAN |

Cell-Specific Variations

The CAR components and construction methods disclosed above are suitable for use in T cells and other immune effector cells, but are not exhaustive. Certain variations may be useful in subsets of cells, and are known in the art.

For example, in NK cells, the TM domain may be chosen or adapted from NKG2D, FcγRIIIa, NKp44, NKp30, NKp46, actKIR, NKG2C, or CD8α. NK cells also express a number of transmembrane adapters that are triggered via association with activating receptors, providing an NK cell specific signal enhancement. For example, the TM adapter can be chosen or adapted from FceR1γ (ITAMx1), CD3ζ (ITAMx3), DAP12 (ITAMx1), or DAP10 (YxxM/YINM). In certain embodiments, the TM domains and adapters may be paired, e.g.: NKG2D and DAP10, FcγRIIIa and CD3ζ or FceR1γ, NKp44 and DAP12, NKp30 and CD3ζ or FceR1γ, NKp46 and CD3ζ or FceR1γ, actKIR and DAP12, and NKG2C and DAP12.

In certain embodiments, in NK cells, the hinge domain may be chosen or adapted from, e.g., NKG2, TMα, or CD8.

In certain embodiments, in NK cells, the intracellular signaling and/or costimulatory domain may comprise one or more of: CD137/41BB (TRAF, NFkB), DNAM-1 (Y-motif), NKp80 (Y-motif), 2B4 (SLAMF)::ITSM, CRACC (CS1/SLAMF7)::ITSM, CD2 (Y-motifs, MAPK/Erk), CD27 (TRAF, NFkB); one or more integrins (e.g., multiple integrins); a cytokine receptor associated with persistence, survival, or metabolism, such as IL-2/15Rbyc::Jak1/3, STAT3/5, PI3K/mTOR, and MAPK/ERK; a cytokine receptor associated with activation, such as IL-18R::NFkB. a cytokine receptor associated with IFN-γ production, such as IL-12R::STAT4; a cytokine receptor associated with cytotoxicity or persistence, such as IL-21R::Jak3/Tyk2, or STAT3; and a TM adapter, as disclosed above. In some embodiments, the NK cell CAR comprises three signaling domains, a TM domain, and optionally, a TM adapter.

The choice of costimulatory domain may also depend on the phenotype or subtype of the NK cell; for example, in some experiments, 4-1BB may be effective as a costimulatory domain in memory-like (ML) NK cells (including CIMLs) but less efficacious in NK cells. Additionally, signaling domains that may be harnessed that are more selectively expressed in ML NK cells include DNAM-1, CD137, and CD2.

Immune Effector Cells

Immune effector cells as disclosed herein may include T cells, NK cells, iNKT cells, and others, for example macrophages, and subtypes thereof.

Any of these immune effector cells may be transduced with a CAR using techniques known in the art. The resulting CAR-bearing immune effector cells may be used in the immunotherapy of disease, for example cancer, by adoptive cell transfer (ACT) into a subject in need. CAR-bearing immune effector cells include CAR-T cells, CAR-NK cells (and subtypes thereof, such as CAR-ML NK cells and CAR-CIMLs), CAR-iNKT cells, and CAR-macrophages.

Immune effector cells for use in ACT may be autologous or allogeneic. In some embodiments, the use of allogeneic cells permits deliberate polymorphic mismatch between donor and recipient, which offers certain advantages discussed below.

### T Cells

T cells are immune cells which express a T cell receptor (TCR) on their surface. Effector T cells include cytotoxic (CD8+) T cells, helper (CD4+) T cells, viral-specific cytotoxic T cells, memory T cells, gamma delta (γδ) T cells.

T cells may be primary T cells, or may be derived from progenitor cells. T cells can be derived from various sources, including peripheral or cord blood cells, stem cells, or induced pluripotent stem cells (iPSCs), Methods of enriching/isolating, differentiating, and otherwise producing T cells are known in the art.

iNKT Cells

Invariant natural killer T cells, also called iNKT cells or type-I NKT cells, represent a distinct lymphocyte population, characterized by expression of an invariant T cell receptor α-chain and certain TCR β-chains (Vα24-Jα18 combined with Vβ11). iNKT TCR-mediated responses are restricted by CD1d, a member of the non-polymorphic CD1 antigen presenting protein family, which promotes the presentation of endogenous and pathogen-derived lipid antigens to the TCR. The prototypical ligand for invariant receptor is α-galactosylceramide (αGalCer). Upon binding of the invariant TCR to CD1d-αGalCer, iNKT will expand. The CD1d gene is monomorphic and expressed by only a few cell types, limiting the potential toxicity of NKT cells in the autologous or allogeneic settings.

NK Cells

Natural killer (NK) cells are traditionally considered innate immune effector lymphocytes which mediate host defense against pathogens and antitumor immune responses by targeting and eliminating abnormal or stressed cells not by antigen recognition or prior sensitization, but through the integration of signals from activating and inhibitory receptors. Natural killer (NK) cells are an alternative to T cells for allogeneic cellular immunotherapy since they have been administered safely without major toxicity, do not cause graft versus host disease (GvHD), naturally recognize and eliminate malignant cells, and are amendable to cellular engineering.

NK cells may be primary NK cells, or may be derived from progenitor cells. NK cells can be derived from various sources, including peripheral or cord blood cells, stem cells, or induced pluripotent stem cells (iPSCs), Methods of enriching/isolating, differentiating, and otherwise producing NK cells are known in the art.

Memory-Like NK Cells

In addition to their innate cytotoxic and immunostimulatory activity, NK cells constitute a heterogeneous and versatile cell subset, including persistent memory-like NK populations that mount a robust recall response. ML-NK cells can be produced by stimulation by pro-inflammatory cytokines or activating receptor pathways, either naturally or artificially. ML-NK cells produced by cytokine activation have been used clinically in the setting of leukemia immunotherapy.

Increased CD56, Ki-67, NKG2A, and increased activating receptors NKG2D, NKp30, and NKp44 have been observed in in vivo differentiated ML NK cells. In addition, in vivo differentiation showed modest decreases in the median expression of CD16 and CD11b. Increased frequency of TRAIL, CD69, CD62L, NKG2A, and NKp30-positive NK cells were observed in ML NK cells compared with both ACT and BL NK cells, whereas the frequencies of CD27+ and CD127+ NK cells were reduced. Finally, unlike in vitro differentiated ML NK cells, in vivo differentiated ML NK cells did not express CD25.

Cytokine-Induced Memory-Like Natural Killer Cells (CIML-NKs)

NK cells may be induced to acquire a memory-like phenotype, for example by preactivation with combinations of cytokines, such as interleukin-12 (IL-12), IL-15, and IL-18. These cytokine-induced memory-like (CIML) NK cells (CIML-NKs or CIMLs) exhibit enhanced response upon restimulation with the cytokines or triggering via activating receptors. CIML NK cells may be produced by activation with cytokines such as IL-12, IL-15, and IL-18 and their related family members, or functional fragments thereof, or fusion proteins comprising functional fragments thereof.

CIML NK cells may be identified by their method of production. CIML cells can be produced by differentiated cytokine-activated (i.e., CIML) NK cells.

CIML NK cells typically exhibit differential cell surface protein expression patterns when compared to traditional NK cells. Such expression patterns are known in the art and may comprise, for example, increased CD56, CD56 subset CD56dim, CD56 subset CD56bright, CD16, CD94, NKG2A, NKG2D, CD62L, CD25, NKp30, NKp44, and NKp46 (compared to control NK cells) in CIML NK cells (see e.g., Romee et al. Sci Transl Med. 2016 Sep. 21; 8(357):357). Memory-like (ML) and cytokine induced memory-like (CIML) NK cells may also be identified by observed in vitro and in vivo properties, such as enhanced effector functions such as cytotoxicity, improved persistence, increased IFN-γ production, and the like.

NK cells can be activated using cytokines, such as IL-12/15/18. The NK cells can be incubated in the presence of the cytokines for an amount of time sufficient to form cytokine-induced memory-like (CIML) NK cells. Such techniques are known in the art.

CD33, FTL-3, and CLL-1-Specific Chimeric Antigen Receptors (CARs)

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). Disclosed herein are CD33, FTL-3, and CLL-1-specific chimeric antigen receptor (CAR) that can be that can be expressed in immune effector cells to enhance antitumor activity against CD33, FTL-3, and CLL-1-expressing tumor cells.

As discussed above, the disclosed CAR generally comprises: an extracellular ligand binding domain, a hinge domain, a transmembrane domain, a cytoplasmic signaling domain, and optionally a co-stimulatory domain. The extracellular ligand binding domain comprises the CD33-binding region and is responsible for antigen recognition. In another embodiment, the extracellular ligand binding domain comprises a FLT3-binding region. In yet another embodiment, the extracellular ligand binding domain comprises a CLL-1 binding region. The transmembrane domain connects the extracellular ligand binding domain to the cytoplasmic signaling domain and resides within the cell membrane when expressed by a cell. The cytoplasmic signaling domain transmits an activation signal to the immune effector cell after antigen recognition. For example, the cytoplasmic signaling domain may optionally contain costimulatory protein domains, such as CD28, 41BB, and ICOS, that are able to enhance T-cell activation by T-cell receptors.

### Antibodies

Provided herein are antibodies comprising the polypeptides disclosed herein. In some embodiments the antibodies comprise the V_{H} and V_{L} chains disclosed herein.

Various forms of antibodies disclosed are contemplated herein. For example, the antibodies can have human frameworks and constant regions of the isotypes, IgA, IgD, IgE, IgG, and IgM, more particularly, IgG1, IgG2, IgG3, IgG4, and in some cases with various mutations to alter Fc receptor function or prevent Fab arm exchange or an antibody fragment, e.g., a F(ab′)2 fragment, a F(ab) fragment, a single chain Fv fragment (scFv), etc.

In certain embodiments, an antibody provided herein is a human antibody. Human antibodies can be produced using various techniques known in the art. For example, human antibodies may also be generated by isolating Fv clone variable domain sequences selected from human-derived phage display libraries. Such variable domain sequences may then be combined with a desired human constant domain.

An antibody as provided herein may be a chimeric antibody, e.g. comprising a non-human variable region (e.g., a variable region derived from a mouse, rat, hamster, rabbit, or non-human primate, such as a monkey) and a human constant region, or a “class switched” antibody in which the class or subclass has been changed from that of the parent antibody.

An antibody as provided herein may be a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Antibodies disclosed herein may also be bispecific or trispecific—i.e., that comprise an antigen-recognition domain that comprises one of the polypeptides disclosed herein and one or more other antigen-recognition domains that binds to another antigen. For example, one arm of the antibody may bind a polymorph of an antigen on an AML cell, and the other arm may bind CD3 or another T-cell target to bring T-cells in proximity to tumor cells. In an example of a trispecific antibody, the antibody would also bind another target on T-cell such as CD28 to enhance activity and persistence of recruited T-cells.

In some embodiments, a humanized antibody comprises, in addition to the variable regions, a human acceptor framework, e.g. a human immunoglobulin framework or a human consensus framework. Human framework regions that may be used for humanization include but are not limited to framework regions selected using the “best-fit” method, framework regions derived from the consensus sequence of human antibodies of a particular subgroup of light or heavy chain variable regions, human mature (somatically mutated) framework regions or human germline framework regions, and framework regions derived from screening FR libraries.

In certain embodiments, an antibody provided herein is a multispecific antibody, e.g. a bispecific antibody. Multispecific antibodies are monoclonal antibodies that have binding specificities for at least two different sites. For example, one of the binding specificities is for CD33 and the other is for any other antigen. In certain embodiments, bispecific antibodies may bind to two different epitopes of the same antigen. Bispecific antibodies may also be used to localize cytotoxic agents to cells which express a target antigen. Bispecific antibodies can be prepared as full length antibodies or antibody fragments. Techniques for making multispecific antibodies include, but are not limited to, recombinant co-expression of two immunoglobulin heavy chain-light chain pairs having different specificities, “knob-in-hole” engineering, engineering electrostatic steering effects for making antibody Fc-heterodimeric molecules, cross-linking two or more antibodies or fragments, using leucine zippers to produce bi-specific antibodies, using “diabody” technology for making bispecific antibody fragments, and using single-chain Fv (sFv) dimers.

Amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Sites of interest for substitutional mutagenesis include the variable regions and framework regions. Amino acids may be grouped according to common side-chain properties:
(1) hydrophobic: Norleucine, Met, Ala, Val, Leu, He;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Amino acid substitutions may be introduced into an antibody of interest and the products screened for a desired activity, e.g., retained/improved antigen binding, decreased immunogenicity, or improved ADCC or CDC. Conservative substitutions are known in the art and are preferred. Non-conservative substitutions will entail exchanging a member of one of these classes for another class.

Antibodies may also comprise modifications to glycan chains substituting certain residues such as Asn 297. For example, antibodies may be engineered or treated to be afucosylated to improve ADCC.

Antibodies comprising the CDRs, variable heavy and light chains disclosed herein may be made by methods known in the art.

For example, variable antibody domains may be cloned into IgG expression vectors (IgG conversion). PCR-amplified DNA fragments of heavy and light chain V-domains may be inserted in frame into, e.g., a human IgG1 constant heavy chain containing recipient mammalian expression vector. Antibody expression may be driven by an MPSV promoter and transcription terminated by a synthetic polyA signal sequence located downstream of the CDS.

Antibodies may be produced using recombinant methods and compositions. Nucleic acids encoding the antibodies described herein are provided. Such a nucleic acid may encode an amino acid sequence comprising the V_{L} and/or an amino acid sequence comprising the V_{H} of the antibody (e.g., the light and/or heavy chains of the antibody). Expression vectors comprising (i.e., transformed with) such nucleic acids are provided, as are host cells comprising such nucleic acids. In one such embodiment, a host cell comprises (1) a vector comprising a nucleic acid that encodes an amino acid sequence comprising the V_{L} and an amino acid sequence comprising the V_{H}, or (2) a first vector comprising a nucleic acid that encodes an amino acid sequence comprising the V_{L} of the antibody and a second vector comprising a nucleic acid that encodes an amino acid sequence comprising the V_{H} of the antibody.

The host cell may be eukaryotic, e.g. a Chinese Hamster Ovary (CHO) cell or lymphoid cell (e.g., Y0, NS0, Sp20 cell). Host cells comprising a nucleic acid encoding the antibody may be cultured under conditions suitable for expression, and the antibody recovered from the host cell or culture medium.

Suitable host cells for cloning or expression of antibody-encoding vectors include other prokaryotic or eukaryotic cells described herein. For example, antibodies may be produced in bacteria (e.g., E. coli), in particular when glycosylation and Fc effector function are not needed. In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. Additional suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts.

In some embodiments, an antibody provided herein has a dissociation constant (Kd) of <1 μM, <100 nM, <50 nM, <10 nM, <5 nM, <1 nM, <0.1 nM, <0.01 nM, or <0.001 nM, and optionally is >10⁻¹³ M. (e.g. 10⁻⁸ M or less, e.g. from 10⁻⁸ M to 10⁻¹³ M, e.g., from 10⁻⁹ M to 10⁻¹³ M). In one embodiment, Kd is measured by a radiolabeled antigen binding assay (RIA) performed with the Fab version of an antibody of interest and its antigen, or using a surface plasmon resonance assay, e.g., WO2015089344.

### Antibody-Drug Conjugates

Also provided herein are immunoconjugates comprising an antibody as disclosed herein, or an antigen-binding fragment thereof, conjugated to one or more drugs (e.g., cytotoxic agents such as chemotherapeutic agents, growth inhibitory agents, toxins, or radioactive isotopes). Immunoconjugates allow for the targeted delivery of a drug or other cytotoxic agent to a tumor, enhancing the therapeutic index by maximizing efficacy and minimizing off-target toxicity. Antibody-drug conjugates (ADCs) disclosed herein include those with anticancer activity. The antibody may be covalently attached to the drug moiety through a linker.

An exemplary embodiment of an ADC comprises: an antibody (Ab), or an antigen-binding fragment thereof, which targets a tumor cell, a cytotoxic moiety such as a drug (D), and a linker moiety (L) that attaches Ab to D. In some embodiments, the antibody is attached to the linker moiety (L) through one or more amino acid residues, such as lysine and/or cysteine.

An ADC may have Formula I:

Ab-(L-D)ₚ

wherein:
Ab is an antibody as disclosed herein, or an antigen-binding fragment thereof;
L is a linker;
D is a drug; and
p is about 1 to about 20.

The antibody (Ab) may comprise a polypeptide disclosed herein.

The drug moiety (D) of the ADC may include any compound, moiety or group that has a cytotoxic or cytostatic effect, or may be a diagnostic or detectable agent.

The linker (L) is a bifunctional or multifunctional moiety that has, e.g., reactive functionalities for attaching to the drug and to the antibody. A linker may have a functionality that is capable of reacting with a free cysteine present on an antibody to form a covalent bond, or a functionality that is capable of reacting with an electrophilic group present on an antibody. Linkers can be susceptible to cleavage (cleavable linker), such as, acid-induced cleavage, photo-induced cleavage, peptidase-induced cleavage, esterase-induced cleavage, and disulfide bond cleavage, at conditions under which the compound or the antibody remains active. Alternatively, linkers can be substantially resistant to cleavage (e.g., stable linker or noncleavable linker). In some aspects, the linker is a procharged linker, a hydrophilic linker, or a dicarboxylic acid based linker.

Examples of cleavable linkers include acid-labile linkers (e.g., comprising hydrazone), protease-sensitive (e.g., peptidase-sensitive) linkers, photolabile linkers, or disulfide-containing linkers. The linker may be, for example, any one of N-succinimidyl-4-(2-pyridyldithio)2-sulfo-butanoate (sulfo-SPDB), N-succinimidyl-3-(2-pyridyldithio)propionate (SPDP), N-succinimidyl 4-(2-pyridyldithio)pentanoate (SPP), N-succinimidyl 4-(2-pyridyldithio)butanoate (SPDB), N-succinimidyl iodoacetate (SIA), N-succimmidyl(4-iodoacetyl)aminobenzoate (SIAB), maleimide PEG NHS, N-succinimidyl 4-(maleimidomethyl)cyclohexanecarboxylate (SMCC), N-sulfosuccinimidyl 4-(maleimidomethyl)cyclohexanecarboxylate (sulfo-SMCC) and 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CXl-1).

The number of drug moieties (e.g., p) that can be conjugated to an antibody may be limited by the number of free cysteine residues (which may be naturally occurring or introduced into the antibody amino acid sequence, or generated using reducing conditions prior to conjugation). In some embodiments, p may be 1 to 10, 2 to 8, or 2 to 5. In some embodiments, p is 3 to 4.

In some embodiments, the drug moiety (D) may be chosen from an anti-cancer agent, anti-hematological disorder agent, an autoimmune treatment agent, an antiinflammatory agent, an antifungal agent, an antibacterial agent, an anti-parasitic agent, an anti-viral agent, an anesthetic agent, a cytotoxin, or a radiotoxin.

In some embodiments, D may be a maytansinoid, a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder and a DHFR inhibitor.

In some embodiments, the drug (D) may be an anticancer agent. Anti-cancer agents include, and D may be, for example:
1) inhibitor or modulator of a protein involved in one or more of the DNA damage repair (DDR) pathways such as:
   a. PARP1/2, including, but not limited to: olaparib, niraparib, rucaparib;
   b. checkpoint kinase 1 (CHK1), including, but not limited to: UCN-01, AZD7762, PF477736, SCH900776, MK-8776, LY2603618, V158411, and EXEL-9844;
   c. checkpoint kinase 2 (CHK2), including, but not limited to: PV1019, NSC 109555, and VRX0466617;
   d. dual CHK1/CHK2, including, but not limited to: XL-844, AZD7762, and PF-473336;
   e. WEE1, including, but not limited to: MK-1775 and PD0166285;
   f. ATM, including, but not limited to KU-55933,
   g. DNA-dependent protein kinase, including, but not limited to NU7441 and M3814; and
   h. Additional proteins involved in DDR;
2) an inhibitor or modulator of one or more immune checkpoints, including, but not limited to:
   a. a PD-1 inhibitor such as nivolumab (OPDIVO), pembrolizumab (KEYTRUDA), pidilizumab (CT-011), and AMP-224 (AMPLIMMUNE);
   b. a PD-L1 inhibitor such as Atezolizumab (TECENTRIQ), Avelumab (Bavencio), Durvalumab (Imfinzi), MPDL3280A (Tecentriq), BMS-936559, and MEDI4736;
   c. an anti-CTLA-4 antibodies such as ipilimumab (YERVOY) and CP-675,206 (TREMELIMUMAB);
   d. an inhibitor of T-cell immunoglobulin and mucin domain 3 (Tim-3);
   e. an inhibitor of V-domain Ig suppressor of T cell activation (Vista);
   f. an inhibitor of band T lymphocyte attenuator (BTLA);
   g. an inhibitor of lymphocyte activation gene 3 (LAG3); and
   h. an inhibitor of T cell immunoglobulin and immunoreceptor tyrosine-based inhibitory motif domain (TIGIT);
3) a telomerase inhibitor or telomeric DNA binding compound;
4) an alkylating agent, including, but not limited to: chlorambucil (LEUKERAN), oxaliplatin (ELOXATIN), streptozocin (ZANOSAR), dacarbazine, ifosfamide, lomustine (CCNU), procarbazine (MATULAN), temozolomide (TEMODAR), and thiotepa;
5) a DNA crosslinking agent, including, but not limited to: carmustine, chlorambucil (LEUKERAN), carboplatin (PARAPLATIN), cisplatin (PLATIN), busulfan (MYLERAN), melphalan (ALKERAN), mitomycin (MITOSOL), and cyclophosphamide (ENDOXAN);
6) an anti-metabolite, including, but not limited to: cladribine (LEUSTATIN), cytarbine, (ARA-C), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEV), methotrexate (RHEUMATREX), and raltitrexed;
7) an antimitotic, which are often plant alkaloids and terpenoids, or a derivative thereof including but limited to: a taxane such as docetaxel (TAXITERE), paclitaxel (ABRAXANE, TAXOL), a vinca alkaloid such as vincristine (ONCOVIN), vinblastine, vindesine, and vinorelbine (NAVELBINE);
8) a topoisomerase inhibitor, including, but not limited to: amsacrine, camptothecin (CTP), genistein, irinotecan (CAMPTOSAR), topotecan (HYCAMTIN), doxorubicin (ADRIAMYCIN), daunorubicin (CERUBIDINE), epirubicin (ELLENCE), ICRF-193, teniposide (VUMON), mitoxantrone (NOVANTRONE), and etoposide (EPOSIN);
9) a DNA replication inhibitor, including, but not limited to: fludarabine (FLUDARA), aphidicolin, ganciclovir, and cidofovir;
10) a ribonucleoside diphosphate reductase inhibitor, including, but not limited to: hydroxyurea;
11) a transcription inhibitor, including, but not limited to: actinomycin D (dactinomycin, COSMEGEN) and plicamycin (mithramycin);
12) a DNA cleaving agent, including, but not limited to: bleomycin (BLENOXANE) and idarubicin;
13) an aromatase inhibitor, including, but not limited to: aminoglutethimide, anastrozole (ARIMIDEX), letrozole (FEMARA), vorozole (RIVIZOR), and exemestane (AROMASIN);
14) an angiogenesis inhibitor, including, but not limited to: genistein, sunitinib (SUTENT), and bevacizumab (AVASTIN);
15) an anti-steroid or anti-androgen, including, but not limited to: aminoglutethimide (CYTADREN), bicalutamide (CASODEX), cyproterone, flutamide (EULEXIN), nilutamide (NILANDRON);
16) a tyrosine kinase inhibitor, including, but not limited to: imatinib (GLEEVEC), erlotinib (TARCEVA), lapatininb (TYKERB), sorafenib (NEXAVAR), and axitinib (INLYTA);
17) an mTOR inhibitor, including, but not limited to: everolimus, temsirolimus (TORISEL), and sirolimus;
18) an apoptosis inducer such as cordycepin;
19) a protein synthesis inhibitor, including, but not limited to: clindamycin, chloramphenicol, streptomycin, anisomycin, and cycloheximide;
20) an antidiabetic, including, but not limited to: metformin and phenformin;
21) a cytotoxic antibiotic, including, but not limited to:
   a. tetracyclines, including, but not limited to: doxycycline;
   b. erythromycins, including, but not limited to: azithromycin;
   c. glycylglycines, including, but not limited to: tigecyline;
   d. antiparasitics, including, but not limited to: pyrvinium pamoate;
   e. beta-lactams, including, but not limited to the penicillins and cephalosporins;
   f. an anthracycline antibiotic, including, but not limited to: doxorubicin, daunorubicin, epirubicin, idarubicin, pirarubicin, aclarubicin, and mitoxantrone;
   g. a bleomycin such as the classical bleomycin A2 (BLENOXANE) and pingyangmycin (also known as bleomycin A5)
   h. another antibiotic, including, but not limited to: chloramphenicol, mitomycin C and actinomycin D (dactinomycin, COSMEGEN); and
22) another agent, such as Bacillus Calmette-Gudrin (B-C-G) vaccine; buserelin (ETILAMIDE); chloroquine (ARALEN); clodronate, pamidronate, or another bisphosphonate; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,p′-DDD, LYSODREN); nocodazole; octreotide (SANDOSTATIN); perifosine; porfimer (particularly in combination with photo- and radiotherapy); suramin; tamoxifen; titanocene dichloride; tretinoin; an anabolic steroid such as fluoxymesterone (HALOTESTIN); estrogens such as estradiol, diethylstilbestrol (DES), and dienestrol; a progestin such as medroxyprogesterone acetate (MPA) and megestrol; and testosterone.

In some embodiments, the drug moiety (D) may be a toxin. Plant-derived protein toxins include ribosome inactivating proteins (RIPs) such as shiga toxins, type I (e.g. trichosanthin and luffin) and type II (e.g. ricin, agglutinin, and abrin), as well as saporin, gelonin, and pokeweed antiviral protein; and bacterial toxins include Pseudomonas exotoxin and Diphtheria toxin.

In some embodiments, the drug moiety (D) may be a diagnostic or detectable agent. Such immunoconjugates can be useful for monitoring or prognosing the onset, development, progression and/or severity of a disease or disorder as part of a clinical testing procedure, such as determining the efficacy of a particular therapy. Such diagnosis and detection can be accomplished by coupling the antibody to detectable substances including, but not limited to, various enzymes, such as horseradish peroxidase, alkaline phosphatase, beta-galactosidase, or acetylcholinesterase; prosthetic groups, such as, but not limited to, streptavidinfoiotin and avidin/biotin; fluorescent materials, such as, but not limited to, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, umbelliferone, fluorescein, fluorescein isothiocyanate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I, ¹²⁵I, ¹²³I, and ^{m}I), carbon (¹⁴C), sulfur (³⁵S), tritium, indium (¹¹⁵In, ¹¹³In, ¹¹²In, and ^{m}In), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ⁶⁴Cu, ¹¹³Sn, and ¹¹⁷Sn; and positron emitting metals using various positron emission tomographies, and nonradioactive paramagnetic metal ions.

In some embodiments the drug moiety D is chosen from saporin, MMAE, MMAF, DM1, DM4. In some embodiments, the drug is saporin.

### Treatment Applications

The polypeptides, including antibodies and functional antigen-binding fragments thereof, CAR-bearing immune effector cells, and compositions described herein, antibody-drug conjugates, and pharmaceutical compositions comprising them can be used in the treatment or prevention of progression of proliferative diseases such as cancers and myelodysplastic syndromes. The cancer may be a hematologic malignancy or solid tumor. Hematologic malignancies include leukemias, lymphomas, multiple myeloma, and subtypes thereof. Lymphomas can be classified various ways, often based on the underlying type of malignant cell, including Hodgkin's lymphoma (often cancers of Reed-Sternberg cells, but also sometimes originating in B cells; all other lymphomas are non-Hodgkin's lymphomas), non-Hodgkin's lymphomas, B-cell lymphomas, T-cell lymphomas, mantle cell lymphomas, Burkitt's lymphoma, follicular lymphoma, and others as defined herein and known in the art. Myelodysplastic syndromes comprise a group of diseases affecting immature leukocytes and/or hematopoietic stem cells (HSCs); MDS may progress to AML.

B-cell lymphomas include, but are not limited to, diffuse large B-cell lymphoma (DLBCL), chronic lymphocytic leukemia (CLL)/small lymphocytic lymphoma (SLL), and others as defined herein and known in the art.

T-cell lymphomas include T-cell acute lymphoblastic leukemia/lymphoma (T-ALL), peripheral T-cell lymphoma (PTCL), T-cell chronic lymphocytic leukemia (T-CLL), Sezary syndrome, and others as defined herein and known in the art.

Leukemias include acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) hairy cell leukemia (sometimes classified as a lymphoma), and others as defined herein and known in the art.

Plasma cell malignancies include lymphoplasmacytic lymphoma, plasmacytoma, and multiple myeloma.

Solid tumors include melanomas, neuroblastomas, gliomas or carcinomas such as tumors of the brain, head and neck, breast, lung (e.g., non-small cell lung cancer, NSCLC), reproductive tract (e.g., ovary), upper digestive tract, pancreas, liver, renal system (e.g., kidneys), bladder, prostate and colorectum.

Methods described herein are generally performed on a subject in need thereof. A subject in need of the therapeutic methods described herein can be a subject having, diagnosed with, suspected of having, or at risk for developing, or at rick of progressing to a later stage of, cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans, or other animals such as chickens. For example, the subject can be a human subject.

Generally, a safe and effective amount of a therapy, e.g. an antibody or functional antigen-binding fragment thereof, CAR-bearing immune effector cell, or antibody-drug conjugate, is, for example, an amount that would cause the desired therapeutic effect in a subject while minimizing undesired side effects.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration. Where the product is, for example, a biologic or cell therapy, the mode of administration will likely be via injection or infusion.

Standards of Care and Conditioning Regimens for Immunotherapy

Standard of care treatment for cancers, such as AML, can involve anti-cancer pharmaceutical therapy including chemotherapy and targeted therapy, as well as hematopoietic stem cell transplant (HSCT).

For example, the combination of cytarabine (cytosine arabinoside or ara-C) and an anthracycline such as daunorubicin (daunomycin) or idarubicin is the first-line chemotherapy for AML. Other chemotherapeutics that may be used to treat AML include cladribine (Leustatin, 2-CdA), fludarabine (Fludara), mitoxantrone, Etoposide (VP-16), 6-thioguanine (6-TG), hydroxyurea, corticosteroids such as prednisone or dexamethasone, methotrexate (MTX), 6-mercaptopurine (6-MP), azacitidine (Vidaza), and decitabine (Dacogen). In addition, targeted therapies may be used in appropriate patients, such as midostaurin (Rydapt) or gilteritinib (Xospata) in patients with FLT-3 mutations; gemtuzumab ozogamicin (Mylotarg) in CD33-positive AML; BCL-2 inhibitor such as venetoclax (Venclexta); IDH inhibitors such as ivosidenib (Tibsovo) or enasidenib (Idhifa); and hedgehog pathway inhibitors such as glasdegib (Daurismo). Although the rate of complete remission can be as high as 80% following initial induction chemotherapy, the majority of AML patients will eventually progress to relapsed or refractory (RR) disease, and five-year survival rate are about 35% in people under 60 years old and 10% in people over 60 years old. See, Walter R B et al., “Resistance prediction in AML: analysis of 4601 patients from MRC/NCRI, HOVON/SAKK, SWOG and MD Anderson Cancer Center,” Leukemia 29(2):312-20 (2015) and Döhner, H et al., “Acute Myeloid Leukemia,” NEJM 373 (12): 1136-52 (2015).

Adoptive cell transfer (ACT) therapy is also possible in the treatment of cancers such as AML, either with or without a conditioning regimen. Currently, hematopoietic stem cell transfer (HSCT) is used; other therapies such as transplant of NK cells, chimeric antigen receptor (CAR) T cells (CAR-T) and other CAR-bearing immune effector cells are in development.

Hematopoietic Stem Cell Transplant (HSCT)

Hematopoietic stem cell transplantation (HSCT) is a potentially curative therapeutic approach for a variety of malignant and nonmalignant hematopoietic diseases, such as AML, CML, ALL, Hodgkin and non-Hodgkin lymphoma, multiple myeloma, myelodysplastic syndrome, neuroblastoma, Ewing sarcoma, gliomas, and solid tumors. HSCT for AML is typically allogeneic and requires HLA-matching between donor and patient for several reasons. The first is to prevent HvGD, but an additional benefit is the graft-versus-leukemia (GvL) effect wherein donor immune cells recognize patient leukemia cells as being foreign to them and attack them. In some cases, for example where the patient may not be able to tolerate an allogeneic transplant, an autologous transplant may be used, often after careful purging to attempt to remove leukemia cells.

Typically, when HSCT is performed in patients with malignant disorders, preparative or conditioning regimens are administered as part of the procedure to effect immunoablation to prevent graft rejection, and to reduce tumor burden. Traditionally, these goals have been achieved by using otherwise supralethal doses of total body irradiation (TBI) and chemotherapeutic agents with nonoverlapping toxicities, so-called “high-intensity” pre-HSCT conditioning. However, as it was recognized that immunologic reactions of donor cells against malignant host cells (i.e., graft-versus-tumor effects) substantially contributed to the effectiveness of HSCT, reduced-intensity and nonmyeloablative conditioning regimens have been developed, making HCT applicable to older and medically infirm patients.

Conditioning regimens are known in the art. See, e.g., Gyurkocza and Sandmaier B M, “Conditioning regimens for hematopoietic cell transplantation: one size does not fit all,” Blood 124(3): 344-353 (2014). Conditioning regimens may be classified as high-dose (myeloablative), reduced-intensity, and nonmyeloablative, following the Reduced-Intensity Conditioning Regimen Workshop, convened by the Center for International Blood and Marrow Transplant Research (CIBMTR) during the Bone Marrow Transplantation Tandem Meeting in 2006.

Immunotherapy with CAR-Bearing Immune Effector Cells

CAR-bearing immune effector cells have been used in treatment of AML with varying results. Clinical trials with CAR-T cells targeting AML antigens such as CD33 and CD123 have been registered and are proceeding, but have not to date seen unequivocal success. One problem is the difficulty in targeting a suitable targetable surface antigen that is not also expressed on healthy cells. CAR-engineered cells from the immortalized NK-92 cell line targeting AML antigen CD33 have also been tested.

There are multiple scenarios where therapy with CAR-bearing immune effector cells would be useful in AML. In one scenario where a patient with AML is treated with CAR cell therapy, the CAR present on the surface of the CAR-bearing immune effector cell recognizes and binds to an AML cell antigen, such as CD33, FLT-3, or CLL-1, and the AML cell is targeted for killing. The CAR cell therapy will also target the same antigens on the patient's own hematopoietic stem cells. Thereafter, the patient receives hematopoietic stem cell transplant (HSCT), optionally undergoing preliminary procedures to extinguish the CAR cells and condition the patient for HSCT beforehand, and the engrafted donor stem cells then attack the remaining AML cells. Although this is an effective therapy for many patients, AML may nevertheless relapse (e.g. in about 50% of cases), and further treatment with the same CAR cell therapy is typically not feasible because the engrafted stem cells and their progeny will recognize the newly-infused CAR cells as foreign and destroy them.

Polymorphic Targeting of Cancer Antigens

Polymorphic Targeting. Another approach to the use of CAR-bearing immune effector cells in the treatment of AML exploits natural variation in AML target antigen polymorphism to solve this problem. Certain AML antigens, such as CD33, FLT-3, and CLL-1 occur as polymorphic variants. For example, in a given population, an AML antigen exists as two predominant polymorphs, e.g. A, in which a given base pair in the genomic sequence of the antigen is A-T, and B, in which the base pair is C-G at the same position. This will lead to a different amino acid residue being translated, and provided that the base pair occurs in a coding region, an antigen with a different amino acid residue and thus a different primary and, thus, tertiary structure. If the change is significant, and the residue is in an solvent-exposed position on the cell surface that is accessible to an antibody, an antigen-binding fragment thereof, or a synthetic antigen-binding protein such as an scFv, then a CAR may be designed to bind a single polymorph selectively over the other(s). And a CAR-T cell, or other immune effector cell, bearing such a selective CAR, can target and kill AML cells of a single polymorphic form. See, e.g., Table 2 below, setting forth three AML antigens and their common polymorphisms:

TABLE 2
Polymorphisms in AML Antigens

| POLYMORPHISM | A | BB |
|---|---|---|
| CD33 | Arg 64.3% | Gly 35.7% |
| ARG69GLY (R69G) | | |
| FLT3 | Thr 40.1% | Met 59.9% |
| THR227MET (T227M) | | |
| CLL1 | Lys 26.3% | Gln 73.7% |
| LYS244GLN (K244Q) | | |

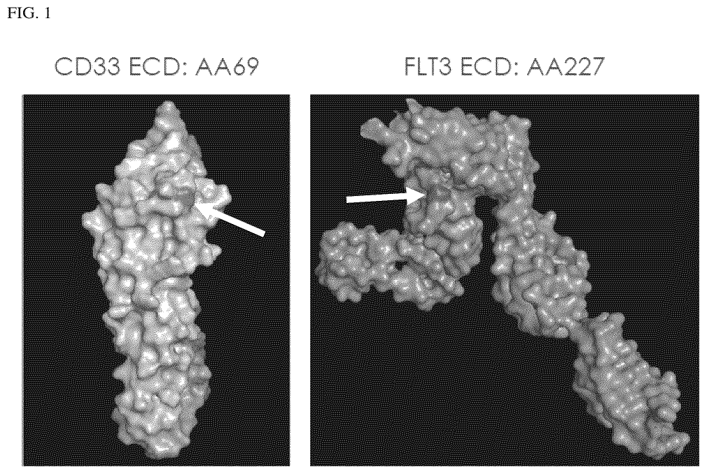

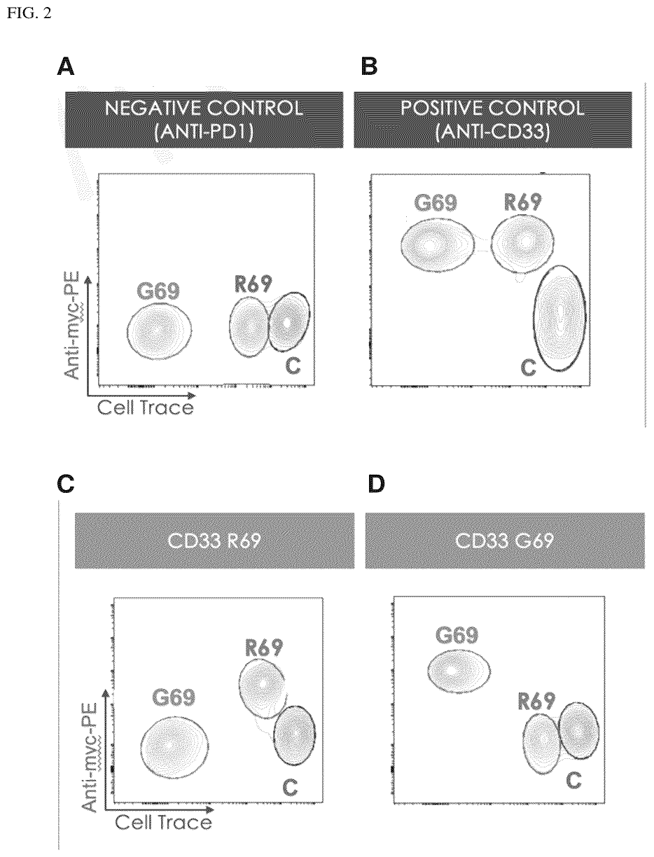

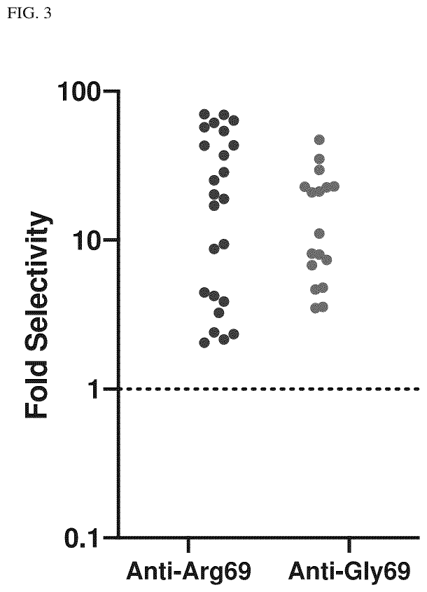

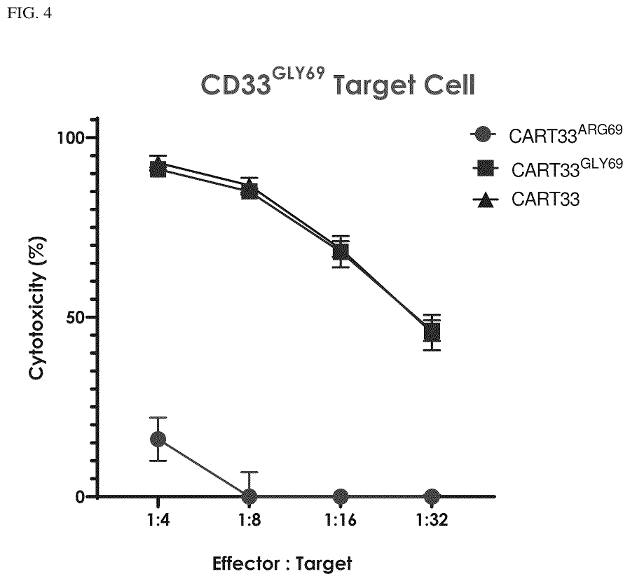

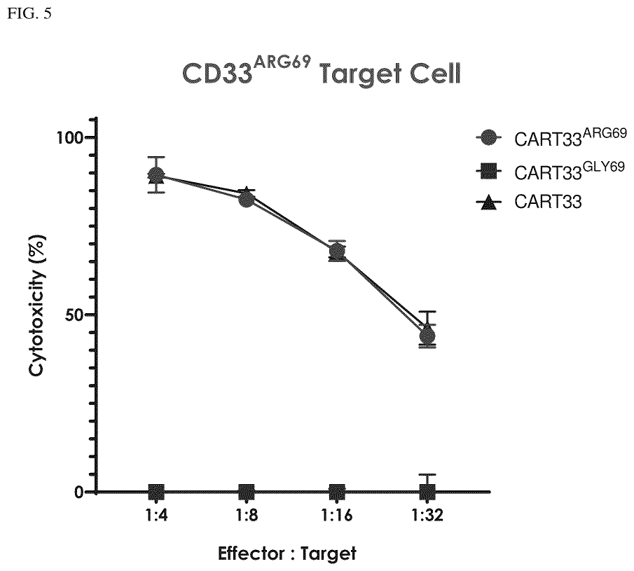

See also FIG. 1 showing the positions of the CD33 extracellular domain with amino acid 69 in the left panel, and FLT3 ECD AA267 in the right panel, each in a relatively solvent-accessible position.

Patient-Donor Mismatch. When the patient has one polymorphic form of an AML antigen, and a donor of cells for use in HSCT has another polymorphic form of the antigen, creating a “mismatch” of AML antigen polymorphisms, several useful treatment scenarios arise.

When the donor provides polymorphically “mismatched” stem cells for HSCT, and those cells are engrafted into a recipient patient, CAR-bearing immune effector cell therapy with a CAR selective for the patient's polymorphic variant may be used—even after HSCT transplant—to target and kill any remaining cells bearing the patient's polymorphic form of the antigen. Because the cells selectively target the patient's polymorphism, the donor's engrafted cells will be spared. Treatment may be either prophylactic, or upon signs of relapsing disease. Thus, relapse is prevented or treated, and the patient can achieve disease-free survival.

The HSC and the T cells or other immune effector cells that will be engineered to express a CAR may both come from the same donor, polymorphically mismatched to the intended recipient. As shown below in Table 3, the donor must be homozygous for either one polymorphism or the other (i.e., cannot be heterozygous), and the receiving patient can be either homozygous for the other polymorphism or heterozygous.

TABLE 3
Treatment Options with Anti-CD33
Polymorphic Antibodies and CARs

| | Donor Genotype | | |
|---|---|---|---|
| Rs2455069 | AA | AG | |
| A > G Arg69Gly | (ARG/ARG) | (ARG/GLY) | GG(GLY/GLY) |
| AA(ARG/ARG) | — | — | CAR-A |
| AG (ARG/GLY) | CAR-G | — | CAR-A |
| GG (GLY/GLY) | CAR-G | — | — |

In another variation, the HSC may come from one mismatched donor, and the immune effector cells that will be engineered to express a CAR will come from a different donor. If the CAR-bearing immune effector cells are CAR-T cells, these cells may have the T-cell receptor disabled, e.g., by genetic disruption of one or more of its components (such as TRAC), e.g., using CRISPR or another genome editing tool, or a technology such as PEBL.

### Pharmaceutical Compositions

Also disclosed is a pharmaceutical composition comprising a disclosed molecule in a pharmaceutically acceptable carrier. Pharmaceutical carriers are known to those skilled in the art. These most typically would be standard carriers for administration of drugs to humans, including solutions such as sterile water, saline, and buffered solutions at physiological pH. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. The solution should be RNAse free. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo or polynucleotide chemistry and hybridization described herein are those well-known and commonly used in the art.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “antibody” refers to a polypeptide that includes canonical immunoglobulin sequence elements sufficient to confer specific binding, or e.g. immune-reacts and/or is directed to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (V_{H}) domain, followed by three constant domains: C_{H}1, C_{H}2, and the carboxy-terminal C_{H}3. A short region, known as the “switch”, connects the heavy chain variable and constant regions. The “hinge” connects C_{H}2 and C_{H}3 domains to the rest of the antibody. Two disulfide bonds in this hinge region connect the two heavy chain polypeptides to one another in an intact antibody. Each light chain is comprised of two domains—an amino-terminal variable (V_{L}) domain, followed by a carboxy-terminal constant (C_{L}) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the C_{H}2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “Complementarity-Determining Regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity.

The term “antigen” refers to a molecular entity that may be soluble or cell membrane bound in particular but not restricted to molecular entities that can be recognized by means of the adaptive immune system including but not restricted to antibodies or TCRs, or engineered molecules including but not restricted to transgenic TCRs, chimeric antigen receptors (CARs), scFvs or multimers thereof, Fab-fragments or multimers thereof, antibodies or multimers thereof, single chain antibodies or multimers thereof, or any other molecule that can execute binding to a structure with high affinity.

The terms “specifically binds” or “specific for” or “specifically recognize” with respect to an antigen-recognizing receptor refer to an antigen-binding domain of said antigen-recognizing receptor which recognizes and binds to a specific polymorphic variant of an antigen, but does not substantially recognize or bind other variants.

The term “monoclonal antibody” (mAb), as applied to the antibodies described in the present disclosure, are compounds derived from a single copy or a clone from any eukaryotic, prokaryotic, or phage clone, and not the method by which it is produced. mAbs of the present disclosure may exist in a homogeneous or substantially homogeneous population.

As used herein, the term “binding affinity” refers to the strength of binding of one molecule to another at a site on the molecule. If a particular molecule will bind to or specifically associate with another particular molecule, these two molecules are said to exhibit binding affinity for each other. Binding affinity is related to the association constant and dissociation constant for a pair of molecules, but it is not critical to the methods herein that these constants be measured or determined. Rather, affinities as used herein to describe interactions between molecules of the described methods are generally apparent affinities (unless otherwise specified) observed in empirical studies, which can be used to compare the relative strength with which one molecule (e.g., an antibody or other specific binding partner) will bind two other molecules (e.g., two versions or variants of a peptide). The concepts of binding affinity, association constant, and dissociation constant are well known.

As used herein, the term “sequence identity” means the percentage of identical nucleotide or amino acid residues at corresponding positions in two or more sequences when the sequences are aligned to maximize sequence matching, i.e., taking into account gaps and insertions. Identity can be readily calculated by known methods. Methods to determine identity are designed to give the largest match between the sequences tested. Moreover, methods to determine identity are codified in publicly available computer programs. Optimal alignment of sequences for comparison can be conducted, for example, by the local homology algorithm of Smith & Waterman, by the homology alignment algorithms, by the search for similarity method or, by computerized implementations of these algorithms (GAP, BESTFIT, PASTA, and TFASTA in the GCG Wisconsin Package, available from Accelrys, Inc. See generally, Altschul, S. F. et al., J. Mol. Biol. 215: 403-410 (1990) and Altschul et al. Nucl. Acids Res. 25: 3389-3402 (1997). One example of an algorithm that is suitable for determining percent sequence identity and sequence similarity is the BLAST algorithm,

An “antibody fragment” refers to a molecule other than an intact antibody that comprises a portion of an intact antibody that binds the antigen to which the intact antibody binds. Several examples of antibody fragments include but are not limited to Fv, Fab, Fab′, Fab′-SH, F(ab′)₂, diabodies, linear antibodies, single chain variable fragments (scFvs), and multi-specific antibodies formed from antibody fragments. In some embodiments, the antibody fragment is an antigen-binding fragment.

Reviews of current methods for antibody engineering and improvement can be found in R. Kontermann and S. Dubel, (2010) Antibody Engineering Vols. 1 and 2, Springer Protocols, 2^{nd} Edition and W. Strohl and L. Strohl (2012) Therapeutic antibody engineering: Current and future advances driving the strongest growth area in the pharmaceutical industry, Woodhead Publishing. Methods for producing and purifying antibodies and antigen-binding fragments are well known in the art and can be found, in Harlow and Lane (1988) Antibodies: A Laboratory Manual, Cold Spring Harbor Laboratory Press, Cold Spring Harbor, NY, chapters 5-8 and 15.

A “diseased cell” refers to the state of a cell, tissue or organism that diverges from the normal or healthy state and may result from the influence of a pathogen, a toxic substance, irradiation, or cell internal deregulation. A “diseased cell” may also refer to a cell that has been infected with a pathogenic virus. Further the term “diseased cell” may refer to a malignant cell or neoplastic cell that may constitute or give rise to cancer in an individual.

The term “cancer” is known medically as a malignant neoplasm. Cancer is a broad group of diseases involving upregulated cell growth. In cancer, cells (cancerous cells) divide and grow uncontrollably, forming malignant tumors, and invading nearby parts of the body. The cancer may also spread to more distant parts of the body through the lymphatic system or bloodstream. There are over 200 different known cancers that affect humans.

The term “malignant” or “malignancy” describes cells, groups of cells or tissues that constitute a neoplasm, are derived from a neoplasm or can be the origin of new neoplastic cells. The term is used to describe neoplastic cells in contrast to normal or healthy cells of a tissue. A malignant tumor contrasts with a non-cancerous benign tumor in that a malignancy is not self-limited in its growth, is capable of invading into adjacent tissues, and may be capable of spreading to distant tissues. A benign tumor has none of those properties. Malignancy is characterized by anaplasia, invasiveness, and metastasis as well as genome instability. The term “premalignant cells” refer to cells or tissue that is not yet malignant but is poised to become malignant.

The term “chemotherapy” refers to the treatment of cancer (cancerous cells) with one or more cytotoxic anti-neoplastic drugs (“chemotherapeutic agents” or “chemotherapeutic drugs”) as part of a standardized regimen. Chemotherapy may be given with a curative intent or it may aim to prolong life or to palliate symptoms. It is often used in conjunction with other cancer treatments, such as radiation therapy, surgery, and/or hyperthermia therapy. Traditional chemotherapeutic agents act by killing cells that divide rapidly, one of the main properties of most cancer cells. This means that chemotherapy also harms cells that divide rapidly under normal circumstances, such as cells in the bone marrow, digestive tract, and hair follicles. This results in the most common side-effects of chemotherapy, such as myelosuppression (decreased production of blood cells, hence also immunosuppression), mucositis (inflammation of the lining of the digestive tract), and alopecia (hair loss).

The term “immune cell” or “immune effector cell” refers to a cell that may be part of the immune system and executes a particular effector function such as alpha-beta T cells, NK cells (including ML-NKs and CIML-NKs), NKT cells (including iNKT cells), B cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes and macrophages. Preferred immune cells are cells with cytotoxic effector function such as alpha-beta T cells, NK cells (including ML-NKs and CIML-NKs), NKT cells (including iNKT cells), ILC, CIK cells, LAK cells or gamma-delta T cells. “Effector function” means a specialized function of a cell, e.g. in a T cell an effector function may be cytolytic activity or helper activity including the secretion of cytokines.

The term “side-effects” refers to any complication, unwanted or pathological outcome of an immunotherapy with an antigen recognizing receptor that occurs in addition to the desired treatment outcome. The term “side effect” preferentially refers to on-target off-tumor toxicity, that might occur during immunotherapy in case of presence of the target antigen on a cell that is an antigen-expressing non-target cell but not a diseased cell as described herein. A side-effect of an immunotherapy may be the developing of graft versus host disease.

The term “reducing side-effects” refers to the decrease of severity of any complication, unwanted or pathological outcome of an immunotherapy with an antigen recognizing receptor such as toxicity towards an antigen-expressing non-target cell. “Reducing side-effects” also refers to measures that decrease or avoid pain, harm or the risk of death for the patient during the immunotherapy with an antigen recognizing receptor.

The term “combination immunotherapy” refers to the concerted application of two therapy approaches e.g. therapy approaches known in the art for the treatment of disease such as cancer. The term “combination immunotherapy” may also refer to the concerted application of an immunotherapy such as the treatment with an antigen recognizing receptor and another therapy such as the transplantation of hematopoietic cells e.g. hematopoietic cells resistant to recognition by the antigen recognizing receptor. Expression of an antigen on a cell means that the antigen is sufficient present on the cell surface of said cell, so that it can be detected, bound and/or recognized by an antigen-recognizing receptor.

The term “hematopoietic cells”, refers to a population of cells of the hematopoietic lineage capable of hematopoiesis which include but is not limited to hematopoietic stem cells and/or hematopoietic progenitor cells (i.e., capable to proliferate and at least partially reconstitute different blood cell types, including erythroid cells, lymphocytes, and myelocytes). The term “hematopoietic cells” as used herein also includes the cells that are differentiated from the hematopoietic stem cells and/or hematopoietic progenitor cells to form blood cells (i.e. blood cell types, including erythroid cells, lymphocytes, and myelocytes).

A donor hematopoietic cell resistant to recognition of an antigen by an antigen-recognizing receptor means that said cell cannot as easily be detected, bound and/or recognized by an antigen-recognizing receptor specific for said antigen or that the detection, binding and/or recognizing is impaired, so the cell is not killed during immunotherapy.

The term “fratricide” refers to the observation that the antigen associated with disease may be, in addition to diseased cells, present on immune effector cells engineered, such as T cells expressing an antigen-recognizing receptor, such as a CAR. In that case the side-effects of the antigen recognizing receptor will affect the immune effector cells engineered to express the antigen recognizing receptor. Such side-effect is also known in the art as fratricide.

In general, the term “receptor” refers to a biomolecule that may be soluble or attached to the cell surface membrane and specifically binds a defined structure that may be attached to a cell surface membrane or soluble. Receptors include but are not restricted to antibodies and antibody like structures, adhesion molecules, transgenic or naturally occurring TCRs or CARs. In specific, the term “antigen-recognizing receptor” as used herein may be a membrane bound or soluble receptor such as a natural TCR, a transgenic TCR, a CAR, a scFv or multimers thereof, a Fab-fragment or multimers thereof, an antibody or multimers thereof, a bi-specific T cell enhancer (BiTE), a diabody, or any other molecule that can execute specific binding with high affinity.

The term “target” or “target antigen” refers to any cell surface protein, glycoprotein, glycolipid or any other structure present on the surface of the target cell. The term also refers to any other structure present on target cells in particular but not restricted to structures that can be recognized by means of the adaptive immune system including but not restricted to antibodies or TCRs, or engineered molecules including but not restricted to transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

The term “target cells” as used herein refers to cells which are recognized by the antigen-recognizing receptor which is or will be applied to the individual.

The term “system for use in immunotherapy” as used herein refers to the constellation that two kinds of compositions are needed to perform the combined immunotherapy as disclosed herein. Therefore, the system (or set or kit or the combination of compositions) comprises a) an antigen-recognizing receptor wherein said antigen-recognizing receptor specifically recognizes an antigen on target cells in said individual; b) hematopoietic cells resistant to recognition of said antigen by said antigen-recognizing receptor.

“Chimeric antigen receptor” or “CAR” refer to engineered receptors, which graft an antigen specificity onto cells, for example T cells. The CARs disclosed herein comprise an antigen binding domain also known as antigen targeting region, an extracellular spacer domain or hinge region, a transmembrane domain and at least one intracellular signaling domain or at least one co-stimulatory domain and at least one intracellular signaling domain.

In general, a CAR may comprise an extracellular domain (extracellular part) comprising the antigen binding domain, a transmembrane domain and an intracellular signaling domain. The extracellular domain may be linked to the transmembrane domain by a linker. The extracellular domain may also comprise a signal peptide.

A “signal peptide” refers to a peptide sequence that directs the transport and localization of the protein within a cell, e.g. to a certain cell organelle (such as the endoplasmic reticulum) and/or the cell surface.

An “antigen binding domain” refers to the region of the CAR that specifically binds to an antigen (and thereby is able to target a cell containing an antigen). CARs may comprise one or more antigen binding domains. Generally, the targeting regions on the CAR are extracellular. The antigen binding domain may comprise an antibody or an antigen-binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in a scFv the variable portions of an immunoglobulin heavy chain and light chain are fused by a flexible linker to form a scFv. Such a linker may be for example the (GGGG₄S)₃. In some instances, it is beneficial for the antigen binding domain to be derived from the same species in which the CAR will be used in. For example, when it is planned to use it therapeutically in humans, it may be beneficial for the antigen binding domain of the CAR to comprise a human or humanized antibody or antigen-binding fragment thereof. Human or humanized antibodies or fragments thereof can be made by a variety of methods well known in the art.

“Spacer” or “hinge” as used herein refers to the hydrophilic region which is between the antigen binding domain and the transmembrane domain. The CARs disclosed herein may comprise an extracellular spacer domain but is it also possible to pass such a spacer. The spacer may include Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The “transmembrane domain” of the CAR can be derived from any desired natural or synthetic source for such domain. When the source is natural the domain may be derived from any membrane-bound or transmembrane protein. The transmembrane domain may be derived for example from CD8alpha or CD28. When the key signaling and antigen recognition modules are on two (or even more) polypeptides then the CAR may have two (or more) transmembrane domains. Splitting key signaling and antigen recognition modules enables for a small molecule-dependent, titratable and reversible control over CAR cell expression (Wu et al, 2015, Science 350: 293-303) due to small molecule-dependent heterodimerizing domains in each polypeptide of the CAR.

The cytoplasmic domain or the intracellular signaling domain of the CAR is responsible for activation of at least one of the normal effector functions of the immune cell in which the CAR is expressed. “Effector function” means a specialized function of a cell, e.g. in a T cell an effector function may be cytolytic activity or helper activity including the secretion of cytokines. The intracellular signaling domain refers to the part of a protein which transduces the effector function signal and directs the cell expressing the CAR to perform a specialized function.

The intracellular signaling domain may include any complete or truncated part of the intracellular signaling domain of a given protein sufficient to transduce the effector function signal. Prominent examples of intracellular signaling domains for use in the CARs include the cytoplasmic sequences of the T cell receptor (TCR) and co-receptors that act in concert to initiate signal transduction following antigen receptor engagement.

Generally, T cell activation can be mediated by two distinct classes of cytoplasmic signaling sequences, firstly those that initiate antigen-dependent primary activation through the TCR (primary cytoplasmic signaling sequences) and secondly those that act in an antigen-independent manner to provide a secondary or co-stimulatory signal (secondary cytoplasmic signaling sequences, costimulatory signaling domain). Therefore, an intracellular signaling domain of a CAR may comprise a primary cytoplasmic signaling domain and/or a secondary cytoplasmic signaling domain.

Primary cytoplasmic signaling sequences that act in a stimulatory manner may contain ITAMs (immunoreceptor tyrosine-based activation motifs signaling motifs). Examples of ITAM containing primary cytoplasmic signaling sequences often used in CARs are that are those derived from TCR zeta (CD3 zeta), FcR gamma, FcR beta, CD3 gamma, CD3 delta, CD3 epsilon, CD5, CD22, CD79a, CD79b, and CD66d. Most prominent is sequence derived from CD3 zeta.

The cytoplasmic domain of the CAR can be designed to comprise the CD3-zeta signaling domain by itself or combined with any other desired cytoplasmic domain(s). The cytoplasmic domain of the CAR can comprise a CD3 zeta chain portion and a costimulatory signaling region. The costimulatory signaling region refers to a part of the CAR comprising the intracellular domain of a costimulatory molecule. A costimulatory molecule is a cell surface molecule other than an antigen receptor or their ligands that is required for an efficient response of lymphocytes to an antigen. Examples for a costimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C, B7-H3. The cytoplasmic signaling sequences within the cytoplasmic signaling part of the CAR may be linked to each other in a random or specified order. A short oligo- or polypeptide linker, which is preferably between 2 and 10 amino acids in length, may form the linkage. A prominent linker is the glycine-serine doublet.

As an example, the cytoplasmic domain may comprise the signaling domain of CD3-zeta and the signaling domain of CD28. In another example the cytoplasmic domain may comprise the signaling domain of CD3-zeta and the signaling domain of CD27. In an further example, the cytoplasmic domain may comprise the signaling domain of CD3-zeta, the signaling domain of CD28, and the signaling domain of CD27.

As aforementioned either the extracellular part or the transmembrane domain or the cytoplasmic domain of a CAR may also comprise a heterodimerizing domain for the aim of splitting key signaling and antigen recognition modules of the CAR.

A CAR may be designed to comprise any portion or part of the above-mentioned domains as described herein in any combination resulting in a functional CAR.

A “chimeric antigen receptor” has at least an antigen-specific variable region (typically a single chain variable region comprised of antibody heavy and light chain variable regions) linked to an effector cell signaling domain: typically an intracellular domain of a T-cell receptor, exemplified by (but not limited to) the zeta domain of CD3. Upon binding of the antigen-specific region to the corresponding antigen, the signaling domain mediates an effector cell function in the host cell (such as cytotoxicity). The CAR may optionally but does not necessarily comprise additional domains, such as a linker, a transmembrane domain, and other intracellular signaling elements as described above.

The term “genetic modification” or genetically modified” refers to the alteration of the nucleic acid content including but not restricted to the genomic DNA of a cell. This includes but is not restricted to the alteration of a cells genomic DNA sequence by introduction exchange or deletion of single nucleotides or fragments of nucleic acid sequence. The term also refers to any introduction of nucleic acid into a cell independent of whether that leads to a direct or indirect alteration of the cells genomic DNA sequence or not.

The terms “engineered cell” and “genetically modified cell” as used herein can be used interchangeably. The terms mean containing and/or expressing a foreign gene or nucleic acid sequence, which in turn modifies the genotype or phenotype of the cell or its progeny. Especially, the terms refer to the fact that cells can be manipulated by recombinant methods well known in the art to express stably or transiently peptides or proteins, which are not expressed in these cells in the natural state. Genetic modification of cells may include but is not restricted to transfection, electroporation, nucleofection, transduction using retroviral vectors, lentiviral vectors, non-integrating retro- or lentiviral vectors, transposons, designer nucleases including zinc finger nucleases, TALENs or CRISPR/Cas.

The term “therapeutic effective amount” means an amount, which provides a therapeutic benefit.

Immunotherapy is a medical term defined as the “treatment of disease by inducing, enhancing, or suppressing an immune response” Immunotherapies designed to elicit or amplify an immune response are classified as activation immunotherapies, while immunotherapies that reduce or suppress are classified as suppression immunotherapies. Cancer immunotherapy as an activating immunotherapy attempts to stimulate the immune system to reject and destroy tumors. Adoptive cell transfer uses cell-based cytotoxic responses to attack cancer cells Immune cells such as T cells that have a natural or genetically engineered reactivity to a patient's cancer are generated in vitro and then transferred back into the cancer patient.

As used herein, the term “transplant” means administering to a subject a population of donor cells, e.g. hematopoietic cells or CAR-bearing immune effector cells.

The term “treatment” as used herein means to reduce the frequency or severity of at least one sign or symptom of a disease.

As used herein, the term “individual” refers to an animal. Preferentially, the individual is a mammal such as mouse, rat, cow, pig, goat, chicken dog, monkey or human. More preferentially, the individual is a human. The individual may be an individual suffering from a disease such as cancer (a patient), but the subject may be also a healthy subject.

As used herein, the term “fold selective,” means having an affinity for one target (e.g., a first polymorphic variant of an antigen) that is at least x-fold greater than its affinity for another target (e.g., a second polymorphic variant of an antigen), wherein x is at least 2, and may be higher, e.g., 10, 20, 50, 100, or 1000. In preferred embodiments, the fold selectivity is therapeutically meaningful, i.e., sufficient to permit cells expressing one target to be killed and cells bearing the other target to be killed.

### EXAMPLES

### Example 1: Identification of Targets for Polymorphically Selective Polypeptides

Polymorphically selective polypeptides may be identified for antigen targets which, optimally, 1) have a targetable portion in in extracellular domain 2) that is solvent-exposed and accessible to binding by a polymorphically selective polypeptide such as an scFv, 3) has a high population frequency so that donor patient mismatch is possible, and 4) has a high antigen density on target cells.

For example, CD33 ARG69GLY has a high population frequency, with a minor allele frequency (MAF) of 0.42. Similarly, CLL-1 LYS244GLN has a MAF of 0.35, and FLT3 THR227MET has a MAF of 0.40.

### Example 2: Identification of Anti-Human CD33 scFv Clones

Selective anti-human-CD33 scFv clones were discovered by standard screening methodologies of a human antibody library using two recombinant polymorphic forms of human CD33 extracellular domain antigens (CD33^{R69} and CD33^{G69}). Various panning tactics were employed to encourage enrichment of thermostable clones of a desired affinity range. The scFvs were screened for selective binding between two single nucleotide polymorphism (SNP) variants of human CD33 (Arginine 69 and Glycine 69) by flow cytometry and bio-layer interferometry (BLI), for example as described below in Examples 5 and 6. Selected sequences are disclosed below in Polypeptides 1-42.

Additional anti-human-CD33 polypeptides may be identified using these methods.

TABLE 4a
Sequences of Anti-CD33 R69-Selective Polypeptides (CDR Sequences)

| Poly- | | SEQ | | SEQ | | SEQ | | SEQ | | SEQ | | SEQ |
|---|---|---|---|---|---|---|---|---|---|---|---|---|
| peptide | | ID | | ID | | ID | | ID | | ID | | ID |
| No. | HCDR1 | NO | HCDR2 | NO | HCDR3 | NO | LCDR1 | NO | LCDR2 | NO | LCDR3 | NO |
| 1 | YSFTGYYIH | 1 | GWINP | 26 | CARDQ | 51 | RASQTI | 76 | SASTLH | 101 | CQQAY | 126 |
| | | | NSGGT | | WDGYN | | NDWLA | | S | | STPWTF | |
| | | | NYA | | SGYFD | | | | | | | |
| | | | | | YW | | | | | | | |
| 2 | FTFSDYYMS | 2 | SGISGS | 27 | CARTFG | 52 | RASQSI | 77 | TASTLQ | 102 | CQQYD | 127 |
| | | | GYSTY | | RGPDW | | SRYLN | | S | | DLPLTF | |
| | | | YA | | YFDLW | | | | | | | |
| 3 | FTFSNSDMN | 3 | SAISGS | 28 | CARGR | 53 | RASQSI | 78 | GASTL | 103 | CQQSY | 128 |
| | | | GGSTY | | EDDYG | | SSYLN | | HS | | RIPYTF | |
| | | | YA | | DYVFD | | | | | | | |
| | | | | | YW | | | | | | | |
| 4 | GTFSSYAIS | 4 | GWINP | 29 | CAREH | 54 | RASQNI | 79 | GASTR | 104 | CQQYD | 129 |
| | | | NSGNT | | GDMDV | | NSDLA | | AT | | SLPFTF | |
| | | | GYA | | W | | | | | | | |
| 5 | NTFTSYGIS | 5 | GWINP | 30 | CARES | 55 | RSSQSL | 80 | LGSDR | 105 | CMQGL | 130 |
| | | | NSGGT | | WFGEL | | LHSNG | | AS | | QTPITF | |
| | | | KYA | | YYGMD | | YNYLD | | | | | |
| | | | | | VW | | | | | | | |
| 6 | YTFTAYYTH | 6 | GWMNP | 31 | CAREA | 56 | RASQSI | 81 | EASTLE | 106 | CQQAN | 131 |
| | | | NSGHTS | | YDSFD | | SSYLN | | T | | SFPFTF | |
| | | | YA | | YW | | | | | | | |
| 7 | YTFTDYYM | 7 | GWINP | 32 | CARDS | 57 | RASRGI | 82 | GASSLQ | 107 | CQQSY | 132 |
| | H | | NSGGT | | RIAVAA | | NNWLT | | S | | RIPYTF | |
| | | | NYA | | SSFDY | | | | | | | |
| | | | | | W | | | | | | | |
| 8 | FTFSSYAMS | 8 | SDISGS | 33 | CARPGS | 58 | RASQS | 83 | AASSLQ | 108 | CQQSY | 133 |
| | | | GSGTY | | DGEFD | | VSSFLN | | S | | TTPLTF | |
| | | | YA | | YW | | | | | | | |
| 9 | GTFSSDAIN | 9 | GGFDPE | 34 | CARGPS | 59 | RSSRNI | 84 | KASSLE | 109 | CQQAIS | 134 |
| | | | DGETIY | | GYDFEF | | SHWLA | | S | | FPLTF | |
| | | | A | | DYW | | | | | | | |
| 10 | DTFTTYAIS | 10 | GWINP | 35 | CAREGI | 60 | KSSQSV | 85 | WASTR | 110 | CQQYF | 135 |
| | | | NSGVA | | VGATD | | LHSSKN | | ES | | TTPPTF | |
| | | | TYA | | AFDIW | | KNYLA | | | | | |
| 11 | DTFTNHYM | 11 | GWINP | 36 | CARDL | 61 | RASQSL | 86 | AASSLQ | 111 | CQQAN | 136 |
| | H | | NSGGT | | VPAAV | | GSWLA | | S | | SFPLTF | |
| | | | NYA | | GGYFD | | | | | | | |
| | | | | | YW | | | | | | | |
| 12 | FTFSSHWMS | 12 | SAISGS | 37 | CARDD | 62 | QASQDI | 87 | DASNL | 112 | CQQSY | 137 |
| | | | GGSTY | | NSGSQ | | DNYLN | | ET | | STPLTF | |
| | | | YA | | ADW | | | | | | | |
| 13 | YSFTGYYM | 13 | GWINP | 38 | CVKDR | 63 | RASQGI | 88 | AASSLQ | 113 | CQQSY | 138 |
| | H | | NSGGT | | GDRVV | | RNWLA | | S | | RTPYTF | |
| | | | YFA | | TSYLDY | | | | | | | |
| | | | | | W | | | | | | | |
| 14 | YTFTGYYM | 14 | GIINPS | 39 | CARAA | 64 | KSSQSV | 89 | WASTR | 114 | CQQYY | 139 |
| | H | | GGSTSY | | PYYYD | | LYSSNN | | ES | | TTPLTF | |
| | | | A | | SSGYYS | | KNYLA | | | | | |
| | | | | | GGYYF | | | | | | | |
| | | | | | DYW | | | | | | | |
| 15 | FTFSIYEIH | 15 | SAISGS | 40 | CARSY | 65 | RSSQSL | 90 | LASNR | 115 | CKQTS | 140 |
| | | | GGSTY | | CGGDC | | LHSNG | | AS | | HIPLTF | |
| | | | YA | | WDYYY | | YNYLD | | | | | |
| | | | | | YYGMD | | | | | | | |
| | | | | | VW | | | | | | | |
| 16 | FTFSDNSMN | 16 | SYISSS | 41 | CARGR | 66 | RSSQSL | 91 | SASNLQ | 116 | CMQAL | 141 |
| | | | GSTIYY | | ASSWP | | LHSNG | | S | | QTPPTF | |
| | | | A | | NWFDP | | YNYLD | | | | | |
| | | | | | W | | | | | | | |
| 17 | FTFSSYAMS | 17 | SGISYD | 42 | CAREW | 67 | RASQGI | 92 | ESSTLE | 117 | CQQSY | 142 |
| | | | SDKIGY | | EGFDY | | SNNLN | | T | | SAPLTF | |
| | | | A | | W | | | | | | | |
| 18 | YTFTDHYM | 18 | GWINP | 43 | CAKDK | 68 | RSSQSL | 93 | LGSNR | 118 | CMQTL | 143 |
| | H | | NSGGT | | FGDEGS | | LHSNG | | AS | | RTPLTF | |
| | | | NYA | | GWYGD | | YNYLD | | | | | |
| | | | | | FQHW | | | | | | | |
| 19 | FTFSSYWM | 19 | SGFSGS | 44 | CAREW | 69 | RASQNI | 94 | DAKDL | 119 | CQQAN | 144 |
| | H | | ARTYY | | SGFDY | | GPWLA | | HP | | TFPMTF | |
| | | | A | | W | | | | | | | |
| 20 | YMFTGYYIH | 20 | GWINP | 45 | CAKDR | 70 | RASQSI | 95 | GASSLQ | 120 | CQQSY | 145 |
| | | | NSGGT | | FGSGN | | DRWLA | | S | | STPWTF | |
| | | | NYA | | YGYMD | | | | | | | |
| | | | | | VW | | | | | | | |
| 21 | FTFSSYAMS | 21 | SAISGS | 46 | CARELS | 71 | QASQDI | 96 | AASGL | 121 | CQQAN | 146 |
| | | | GGSTY | | HDYGG | | SNNLN | | QS | | SFPLTF | |
| | | | YA | | NSDFD | | | | | | | |
| | | | | | YW | | | | | | | |
| 22 | YTFTDYYIH | 22 | GWINP | 47 | CARDH | 72 | RASRSI | 97 | AASSLQ | 122 | CQQSY | 147 |
| | | | NSGGT | | RIAVAG | | RTWLA | | T | | STPYTF | |
| | | | NYA | | SYFDY | | | | | | | |
| | | | | | W | | | | | | | |
| 23 | YPFTAHYIH | 23 | GWINP | 48 | CARDV | 73 | RASQGI | 98 | DASNL | 123 | CQQAN | 148 |
| | | | NSGGT | | EMATIG | | NNWLA | | ET | | SFPPTF | |
| | | | NYA | | AYWYF | | | | | | | |
| | | | | | DLW | | | | | | | |
| 24 | YSFTSYGIS | 24 | GWISA | 49 | CARAR | 74 | RSSQSL | 99 | DATNL | 124 | CMQAL | 149 |
| | | | YNGNT | | GAGTFF | | LHSNG | | PT | | QTPFTF | |
| | | | NYG | | DYW | | YNYLD | | | | | |
| 25 | YTFTGYYM | 25 | GRINPN | 50 | CARDD | 75 | RASQSI | 100 | AASN | 125 | CQQGY | 150 |
| | H | | GGSTT | | FYYYY | | NDWLA | | LOS | | STPPTF | |
| | | | YA | | LDFW | | | | | | | |

TABLE 4b
Sequences of Anti-CD33 R69-Selective Polypeptides (VH and VL Sequences)

| Polypep- | | SEQ | | SEQ |
|---|---|---|---|---|
| tide No. | Full VH | ID NO | Full VL | ID NO |
| 1 | QVQLVQSGAEVKKPGASVKVS | 151 | DIQMTQSPSSLSASVGDRVTITC | 176 |
| | CKASGYSFTGYYIHWVRQAPG | | RASQTINDWLAWYQQKPGKAP | |
| | QGLEWMGWINPNSGGTNYAQ | | KLLIYSASTLHSGVPSRFSGSGSG | |
| | KFQGRVTMTRDTSTSTVYMEL | | TDFTLTISSLQPEDFATYYCQQA | |
| | SSLRSEDTAVYYCARDQWDGY | | YSTPWTFGQGTKVEIKR | |
| | NSGYFDYWGQGTLVTVSS | | | |
| 2 | EVQLLESGGGLVQPGGSLRLSC | 152 | DIQMTQSPSSLSASVGDRVTITC | 177 |
| | AASGFTFSDYYMSWVRQAPGK | | RASQSISRYLNWYQQKPGKAPK | |
| | GLEWVSGISGSGYSTYYADSV | | LLIYTASTLQSGVPSRFSGSGSGT | |
| | KGRFTISRDNSKNTLYLQMNSL | | DFTLTISSLQPEDFATYYCQQYD | |
| | RAEDTAVYYCARTFGRGPDW | | DLPLTFGGGTKVEIKR | |
| | YFDLWGRGTLVTVSS | | | |
| 3 | EVQLLESGGGLVQPGGSLRLSC | 153 | DIQMTQSPSSLSASVGDRVTITC | 178 |
| | AASGFTFSNSDMNWVRQAPGK | | RASQSISSYLNWYQQKPGKAPK | |
| | GLEWVSAISGSGGSTYYADSV | | LLIYGASTLHSGVPSRFSGSGSGT | |
| | KGRFTISRDNSKNTLYLQMNSL | | DFTLTISSLQPEDFATYYCQQSY | |
| | RAEDTAVYYCARGREDDYGD | | RIPYTFGQGTKLEIKR | |
| | YVFDYWGQGTLVTVSS | | | |
| 4 | QVQLVQSGAEVKKPGASVKVS | 154 | EIVMTQSPATLSVSPGERATLSC | 179 |
| | CKASGGTFSSYAISWVRQAPG | | RASQNINSDLAWYQQKPGQAPR | |
| | QGLEWMGWINPNSGNTGYAQ | | LLIYGASTRATGIPARFSGSGSGT | |
| | KFQGRVTMTRDTSTSTVYMEL | | EFTLTISSLQSEDFAVYYCQQYD | |
| | SSLRSEDTAVYYCAREHGDMD | | SLPFTFGPGTKVDIKR | |
| | VWGQGTTVTVSS | | | |
| 5 | QVQLVQSGAEVKKPGASVKVS | 155 | DIVMTQSPLSLPVTPGEPASISCR | 180 |
| | CKASGNTFTSYGISWVRQAPG | | SSQSLLHSNGYNYLDWYLQKPG | |
| | QGLEWMGWINPNSGGTKYAQ | | QSPQLLIYLGSDRASGVPDRESG | |
| | KFQGRVTMTRDTSTSTVYMEL | | SGSGTDFTLKISRVEAEDVGVYY | |
| | SSLRSEDTAVYYCARESWFGE | | CMQGLQTPITFGQGTRLEIKR | |
| | LYYGMDVWGKGTTVTVSS | | | |
| 6 | QVQLVQSGAEVKKPGASVKVS | 156 | DIQMTQSPSSLSASVGDRVTITC | 181 |
| | CKASGYTFTAYYTHWVRQAP | | RASQSISSYLNWYQQKPGKAPK | |
| | GQGLEWMGWMNPNSGHTSYA | | LLIYEASTLETGVPSRFSGSGSGT | |
| | QKFQGRVTMTRDTSTSTVYME | | DFTLTISSLQPEDFATYYCQQAN | |
| | LSSLRSEDTAVYYCAREAYDSF | | SFPFTFGPGTKVDIKR | |
| | DYWGQGTLVTVSS | | | |
| 7 | QVQLVQSGAEVKKPGASVKVS | 157 | DIQMTQSPSSLSASVGDRVTITC | 182 |
| | CKASGYTFTDYYMHWVRQAP | | RASRGINNWLTWYQQKPGKAP | |
| | GQGLEWMGWINPNSGGTNYA | | KLLIYGASSLQSGVPSRFSGSGSG | |
| | QKFQGRVTMTRDTSTSTVYME | | TDFTLTISSLQPEDFATYYCQQS | |
| | LSSLRSEDTAVYYCARDSRIAV | | YRIPYTFGQGTKLEIKR | |
| | AASSFDYWGQGTLVTVSS | | | |
| 8 | EVQLLESGGGLVQPGGSLRLSC | 158 | DIQMTQSPSSLSASVGDRVTITC | 183 |
| | AASGFTFSSYAMSWVRQAPGK | | RASQSVSSFLNWYQQKPGKAPK | |
| | GLEWVSDISGSGSGTYYADAV | | LLIYAASSLQSGVPSRFSGSGSGT | |
| | KGRFTISRDNSKNTLYLQMNSL | | DFTLTISSLQPEDFATYYCQQSY | |
| | RAEDTAVYYCARPGSDGEFDY | | TTPLTFGQGTKVEIKR | |
| | WGQGTLVTVSS | | | |
| 9 | QVQLVQSGAEVKKPGSSVKVS | 159 | DIQMTQSPSSLSASVGDRVTITC | 184 |
| | CKASGGTFSSDAINWVRQAPG | | RSSRNISHWLA WYQQKPGKAPK | |
| | QGLEWMGGFDPEDGETIYAQK | | LLIYKASSLESGVPSRFSGSGSGT | |
| | FQGRVTITADESTSTAYMELSS | | DFTLTISSLQPEDFATYYCQQAIS | |
| | LRSEDTAVYYCARGPSGYDFE | | FPLTFGGGTKVEIKR | |
| | FDYWGQGTLVTVSS | | | |
| 10 | QVQLVQSGAEVKKPGASVKVS | 160 | DIVMTQSPDSLAVSLGERATINC | 185 |
| | CKASGDTFTTYAISWVRQAPG | | KSSQSVLHSSKNKNYLAWYQQK | |
| | QGLEWMGWINPNSGVATYAN | | PGQPPKLLIYWASTRESGVPDRF | |
| | KFQGRVTMTRDTSTSTVYMEL | | SGSGSGTDFTLTISSLQAEDVAV | |
| | SSLRSEDTAVYYCAREGIVGAT | | YYCQQYFTTPPTFGPGTKVDIKR | |
| | DAFDIWGQGTMVTVSS | | | |
| 11 | QVQLVQSGAEVKKPGASVKVS | 161 | DIQMTQSPSSLSASVGDRVTITC | 186 |
| | CKASGDTFTNHYMHWVRQAP | | RASQSLGSWLAWYQQKPGKAP | |
| | GQGLEWMGWINPNSGGTNYA | | KLLIYAASSLQSGVPSRFSGSGSG | |
| | QKFQGRVTMTRDTSTSTVYME | | TDFTLTISSLQPEDFATYYCQQA | |
| | LSSLRSEDTAVYYCARDLVPA | | NSFPLTFGQGTKVEIKR | |
| | AVGGYFDYWGQGTLVTVSS | | | |
| 12 | EVQLLESGGGLVQPGGSLRLSC | 162 | DIQMTQSPSSLSASVGDRVTITC | 187 |
| | AASGFTFSSHWMSWVRQAPG | | QASQDIDNYLNWYQQKPGKAP | |
| | KGLEWVSAISGSGGSTYYADS | | KLLIYDASNLETGVPSRFSGSGS | |
| | VKGRFTISRDNSKNTLYLQMN | | GTDFTLTISSLQPEDFATYYCQQ | |
| | SLRAEDTAVYYCARDDNSGSQ | | SYSTPLTFGGGTKLEIKR | |
| | ADWGQGTLVTVSS | | | |
| 13 | QVQLVQSGAEVKKPGASVKVS | 163 | DIQMTQSPSSLSASVGDRVTITC | 188 |
| | CKASGYSFTGYYMHWVRQAP | | RASQGIRNWLAWYQQKPGKAP | |
| | GQGLEWMGWINPNSGGTYFA | | KLLIYAASSLQSGVPSRFSGSGSG | |
| | QNFQGRVTMTRDTSTSTVYME | | TDFTLTISSLQPEDFATYYCQQS | |
| | LSSLRSEDTAVYYCVKDRGDR | | YRTPYTFGQGTKLEIKR | |
| | VVTSYLDYWGQGTLVTVSS | | | |
| 14 | QVQLVQSGAEVKKPGASVKVS | 164 | DIVMTQSPDSLAVSLGERATINC | 189 |
| | CKASGYTFTGYYMHWVRQAP | | KSSQSVLYSSNNKNYLAWYQQK | |
| | GQGLEWMGIINPSGGSTSYAQ | | PGQPPKLLIYWASTRESGVPDRF | |
| | KFQGRVTMTRDTSTSTVYMEL | | SGSGSGTDFTLTISSLQAEDVAV | |
| | SSLRSEDTAVYYCARAAPYYY | | YYCQQYYTTPLTFGQGTKLEIKR | |
| | DSSGYYSGGYYFDYWGQGTL | | | |
| | VTVSS | | | |
| 15 | EVQLLESGGGLVQPGGSLRLSC | 165 | DIVMTQSPLSLPVTPGEPASISCR | 190 |
| | AASGFTFSIYEIHWVRQAPGKG | | SSQSLLHSNGYNYLDWYLQKPG | |
| | LEWVSAISGSGGSTYYADSVK | | QSPQLLIYLASNRASGVPDRFSG | |
| | GRFTISRDNSKNTLYLQMNSLR | | SGSGTDFTLKISRVEAEDVGVYY | |
| | AEDTAVYYCARSYCGGDCWD | | CKQTSHIPLTFGQGTKVEIKR | |
| | YYYYYGMDVWGQGTTVTVSS | | | |
| 16 | EVOLVESGGGLVKPGGSLRLS | 166 | DIVMTQSPLSLPVTPGEPASISCR | 191 |
| | CAASGFTFSDNSMNWVRQAPG | | SSQSLLHSNGYNYLDWYLQKPG | |
| | KGLEWVSYISSSGSTIYYADSV | | QSPQLLIYSASNLQSGVPDRFSGS | |
| | KGRFTISRDDSKNTLYLQMNSL | | GSGTDFTLKISRVEAEDVGVYYC | |
| | KTEDTAVYYCARGRASSWPN | | MQALQTPPTFGQGTKLEIKR | |
| | WFDPWGQGTLVTVSS | | | |
| 17 | EVQLLESGGGLVQPGGSLRLSC | 167 | DIQMTQSPSSLSASVGDRVTITC | 192 |
| | AASGFTFSSYAMSWVRQAPGK | | RASQGISNNLNWYQQKPGKAPK | |
| | GLEWVSGISYDSDKIGYADAV | | LLIYESSTLETGVPSRFSGSGSGT | |
| | KGRFTISRDNSKNTLYLQMNSL | | DFTLTISSLQPEDFATYYCQQSYS | |
| | RAEDTAVYYCAREWEGFDYW | | APLTFGGGTKVEIKR | |
| | GQGTLVTVSS | | | |
| 18 | QVQLVQSGAEVKKPGASVKVS | 168 | DIVMTQSPLSLPVTPGEPASISCR | 193 |
| | CKASGYTFTDHYMHWVRQAP | | SSQSLLHSNGYNYLDWYLQKPG | |
| | GQGLEWMGWINPNSGGTNYA | | QSPQLLIYLGSNRASGVPDRFSG | |
| | QKFQGRVTMTRDTSTSTVYME | | SGSGTDFTLKISRVEAEDVGVYY | |
| | LSSLRSEDTAVYYCAKDKFGD | | CMQTLRTPLTFGGGTKVEIKR | |
| | EGSGWYGDFQHWGQGTLVTV | | | |
| | SS | | | |
| 19 | EVQLLESGGGLVQPGGSLRLSC | 169 | DIQMTQSPSSLSASVGDRVTITC | 194 |
| | AASGFTFSSYWMHWVRQAPG | | RASQNIGPWLAWYQQKPGKAP | |
| | KGLEWVSGFSGSARTYYADSV | | KLLIYDAKDLHPGVPSRFSGSGS | |
| | KGRFTISRDNSKNTLYLQMNSL | | GTDFTLTISSLQPEDFATYYCQQ | |
| | RAEDTAVYYCAREWSGFDYW | | ANTFPMTFGQGTRLEIKR | |
| | GQGTLVTVSS | | | |
| 20 | QVQLVQSGAEVKKPGASVKVS | 170 | DIQMTQSPSSLSASVGDRVTITC | 195 |
| | CKASGYMFTGYYIHWVRQAP | | RASQSIDRWLAWYQQKPGKAPK | |
| | GQGLEWMGWINPNSGGTNYA | | LLIYGASSLQSGVPSRFSGSGSGT | |
| | QKFQGRVTMTRDTSTSTVYME | | DFTLTISSLQPEDFATYYCQQSYS | |
| | LSSLRSEDTAVYYCAKDRFGS | | TPWTFGQGTRLEIKR | |
| | GNYGYMDVWGKGTTVTVSS | | | |
| 21 | EVQLLESGGGLVQPGGSLRLSC | 171 | DIQMTQSPSSLSASVGDRVTITC | 196 |
| | AASGFTFSSYAMSWVRQAPGK | | QASQDISNNLNWYQQKPGKAPK | |
| | GLEWVSAISGSGGSTYYADSV | | LLIYAASGLQSGVPSRFSGSGSG | |
| | KGRFTISRDNSKNTLYLQMNSL | | TDFTLTISSLQPEDFATYYCQQA | |
| | RAEDTAVYYCARELSHDYGGN | | NSFPLTFGGGTKVEIKR | |
| | SDFDYWGQGTLVTVSS | | | |
| 22 | QVQLVQSGAEVKKPGASVKVS | 172 | DIQMTQSPSSLSASVGDRVTITC | 197 |
| | CKASGYTFTDYYIHWVRQAPG | | RASRSIRTWLAWYQQKPGKAPK | |
| | QGLEWMGWINPNSGGTNYAQ | | LLIYAASSLQTGVPSRFSGSGSGT | |
| | EFQGRVTMTRDTSTSTVYMEL | | DFTLTISSLQPEDFATYYCQQSYS | |
| | SSLRSEDTAVYYCARDHRIAV | | TPYTFGQGTKLEIKR | |
| | AGSYFDYWGQGTLVTVSS | | | |
| 23 | QVQLVQSGAEVKKPGASVKVS | 173 | DIQMTQSPSSLSASVGDRVTITC | 198 |
| | CKASGYPFTAHYIHWVRQAPG | | RASQGINNWLAWYQQKPGKAP | |
| | QGLEWMGWINPNSGGTNYAQ | | KLLIYDASNLETGVPSRFSGSGS | |
| | KFQGRVTMTRDTSTSTVYMEL | | GTDFTLTISSLQPEDFATYYCQQ | |
| | SSLRSEDTAVYYCARDVEMAT | | ANSFPPTFGQGTKLEIKR | |
| | IGAYWYFDLWGRGTLVTVSS | | | |
| 24 | QVQLVQSGAEVKKPGSSVKVS | 174 | DIVMTQSPLSLPVTPGEPASISCR | 199 |
| | CKASGYSFTSYGISWVRQAPG | | SSQSLLHSNGYNYLDWYLQKPG | |
| | QGLEWLGWISAYNGNTNYGQ | | QSPQLLIYDATNLPTGVPDRFSG | |
| | SLQGRVTITADESTSTAYMELS | | SGSGTDFTLKISRVEAEDVGVYY | |
| | SLRSEDTAVYYCARARGAGTF | | CMQALQTPFTFGQGTKLEIKR | |
| | FDYWGQGTLVTVSS | | | |
| 25 | QVQLVQSGAEVKKPGASVKVS | 175 | DIQMTQSPSSLSASVGDRVTITC | 200 |
| | CKASGYTFTGYYMHWVRQAP | | RASQSINDWLAWYQQKPGKAP | |
| | GQGLEWMGRINPNGGSTTYAQ | | KLLIYAASNLQSGVPSRFSGSGS | |
| | KFQGRVTMTRDTSTSTVYMEL | | GTDFTLTISSLQPEDFATYYCQQ | |
| | SSLRSEDTAVYYCARDDFYYY | | GYSTPPTFGQGTKVEIKR | |
| | YLDFWGKGTTVTVSS | | | |

TABLE 5a
Sequences of Anti-CD33 R69G-Selective Polypeptides (CDR Sequences)

| Poly- | | SEQ | | SEQ | | SEQ | | SEQ | | SEQ | | SEQ |
|---|---|---|---|---|---|---|---|---|---|---|---|---|
| peptide | | ID | | ID | | ID | | ID | | ID | | ID |
| No. | HCDR1 | NO | HCDR2 | NO | HCDR3 | NO | LCDR1 | NO | LCDR2 | NO | LCDR3 | NO |
| 26 | YTFTEN | 201 | GWMN | 218 | CAREG | 235 | QASQDI | 252 | AASSLQ | 269 | CQQTSS | 286 |
| | EMH | | PNSGN | | GDWPY | | RNYLN | | S | | TPLTF | |
| | | | TGYA | | YYMDV | | | | | | | |
| | | | | | W | | | | | | | |
| 27 | YTLTG | 202 | GWMN | 219 | CARASS | 236 | RASQDI | 253 | GASSLQ | 270 | CQQTYS | 287 |
| | YYMH | | PSSGNT | | DRYYY | | RNNLG | | S | | SPPTF | |
| | | | GYA | | DGVWY | | | | | | | |
| | | | | | FDLW | | | | | | | |
| 28 | FTFSTY | 203 | SAISGS | 220 | CARDG | 237 | RASQGI | 254 | QASTLE | 271 | CQQSYS | 288 |
| | AMH | | GGSTY | | YGDYPF | | DNYLA | | S | | IPWTF | |
| | | | YA | | DYW | | | | | | | |
| 29 | YTFTG | 204 | GVINV | 221 | CARVS | 238 | RASQSI | 255 | DASNLE | 272 | CQQGN | 289 |
| | YYLH | | RRGST | | GSYYQP | | SRWLA | | T | | SFPPIF | |
| | | | RYA | | W | | | | | | | |
| 30 | YTFSN | 205 | GWMN | 222 | CVRDG | 239 | RASQSI | 256 | GASSLQ | 273 | CQQTY | 290 |
| | YYMH | | PDSGT | | TMVQGI | | SSWLA | | S | | RTPLTF | |
| | | | TGYA | | FDYW | | | | | | | |
| 31 | GTFSTY | 206 | GGIIPIV | 223 | CARSG | 240 | RASQGI | 257 | GASSVQ | 274 | CQQSYS | 291 |
| | AIT | | GRANY | | GHDLD | | GNDLG | | S | | TPITF | |
| | | | A | | YW | | | | | | | |
| 32 | FTFSSY | 207 | SSISGS | 224 | CARDN | 241 | RASQSV | 258 | ATSTRA | 275 | CQQYG | 292 |
| | GMH | | GDTTY | | PYGDY | | SSSYLA | | T | | SLPLTF | |
| | | | YA | | GGSFDY | | | | | | | |
| | | | | | W | | | | | | | |
| 33 | YTFTSY | 208 | GIIDPS | 225 | CARDY | 242 | RASQGI | 259 | DASNLE | 276 | CQQAN | 293 |
| | YMH | | GGSTN | | YGSGSY | | SNNLN | | T | | SFPLTF | |
| | | | YA | | YGLDY | | | | | | | |
| | | | | | W | | | | | | | |
| 34 | YTFTD | 209 | GIINPS | 226 | CARVD | 243 | RASQGI | 260 | AASTLQ | 277 | CQQSYS | 294 |
| | YYMH | | GGSTR | | GRRWL | | RNDLA | | N | | TPWTF | |
| | | | YA | | QSDYW | | | | | | | |
| 35 | YTFTD | 210 | GIINPS | 227 | CARVD | 244 | RASQGI | 261 | AASTLQ | 278 | CQQSYS | 295 |
| | YYMH | | GGSTR | | GRRWL | | RNDLA | | N | | TPWTF | |
| | | | YA | | RSDYW | | | | | | | |
| 36 | GTFSSY | 211 | GIISPS | 228 | CARTD | 245 | QASQGI | 262 | AASTLQ | 279 | CQQSY | 296 |
| | AIS | | GRSAG | | YGGHK | | NNYLN | | R | | QTPLTF | |
| | | | YG | | WYFDL | | | | | | | |
| | | | | | W | | | | | | | |
| 37 | YTFTG | 212 | GVISPS | 229 | CARAG | 246 | RASQSI | 263 | AASSLQ | 280 | CQQSYS | 297 |
| | YYLH | | GGGTS | | FGEGVF | | SSYLN | | S | | TPLTF | |
| | | | YA | | RHW | | | | | | | |
| 38 | YSFTSH | 213 | GWIKP | 230 | CARGS | 247 | RASQGI | 264 | TASTLQ | 281 | CQQSYS | 298 |
| | AIS | | NSGDT | | DDYYG | | SNYLA | | S | | TPLTF | |
| | | | KYA | | SYYFDY | | | | | | | |
| | | | | | W | | | | | | | |
| 39 | FTFRNY | 214 | SAISGS | 231 | CARVK | 248 | RASQGI | 265 | GASNLE | 282 | CQQAN | 299 |
| | GMG | | GGSTY | | FYGMD | | SNDLA | | T | | SFPFTF | |
| | | | YA | | VW | | | | | | | |
| 40 | YTFTD | 215 | GWMSP | 232 | CARAD | 249 | RVSQGI | 266 | EASTLE | 283 | CQQGY | 300 |
| | YHMH | | NSGNT | | YYGSD | | SSYLN | | S | | STPPTF | |
| | | | GYA | | YVKFD | | | | | | | |
| | | | | | YW | | | | | | | |
| 41 | YTFPN | 216 | GWINP | 233 | CARDR | 250 | RSSQSL | 267 | LGSNRA | 284 | CMQST | 301 |
| | YGIS | | NSGGT | | DILTGY | | LQSNG | | S | | HWPLTF | |
| | | | KYA | | YHFDY | | YNYLD | | | | | |
| | | | | | W | | | | | | | |
| 42 | YTFTD | 217 | GWINP | 234 | CARLN | 251 | RASQGI | 268 | AASSLQ | 285 | CQQSYS | 302 |
| | YFMH | | NSGNT | | DYGDY | | SNNLN | | S | | TPPTF | |
| | | | GYA | | GGPATL | | | | | | | |
| | | | | | DYW | | | | | | | |

TABLE 5b
Sequences of Anti-CD33 R69G-Selective Polypeptides (VH and
VL Sequences)

| Polypep- | | SEQ ID | | SEQ ID |
|---|---|---|---|---|
| tide No. | Full VH | NO | Full VL | NO |
| 26 | QVQLVQSGAEVKKPGASVKV | 303 | DIQMTQSPSSLSASVGDRVTI | 320 |
| | SCKASGYTFTENEMHWVRQA | | TCQASQDIRNYLNWYQQKPG | |
| | PGQGLEWMGWMNPNSGNTG | | KAPKLLIYAASSLQSGVPSRF | |
| | YAQKFQGRVTMTRDTSTSTV | | SGSGSGTDFTLTISSLQPEDFA | |
| | YMELSSLRSEDTAVYYCAREG | | TYYCQQTSSTPLTFGPGTKVD | |
| | GDWPYYYMDVWGKGTTVTV | | IKR | |
| | SS | | | |
| 27 | QVQLVQSGAEVKKPGASVKV | 304 | DIQMTQSPSSLSASVGDRVTI | 321 |
| | SCKASGYTLTGYYMHWVRQ | | TCRASQDIRNNLGWYQQKPG | |
| | APGQGLEWMGWMNPSSGNT | | KAPKLLIYGASSLQSGVPSRF | |
| | GYAQQFQGRVTMTRDTSTST | | SGSGSGTDFTLTISSLQPEDFA | |
| | VYMELSSLRSEDTAVYYCAR | | TYYCQQTYSSPPTFGQGTKLE | |
| | ASSDRYYYDGVWYFDLWGR | | IKR | |
| | GTLVTVSS | | | |
| 28 | EVQLLESGGGLVQPGGSLRLS | 305 | DIQMTQSPSSLSASVGDRVTI | 322 |
| | CAASGFTFSTYAMHWVRQAP | | TCRASQGIDNYLAWYQQKPG | |
| | GKGLEWVSAISGSGGSTYYAD | | KAPKLLIYQASTLESGVPSRF | |
| | SVKGRFTISRDNSKNTLYLQM | | SGSGSGTDFTLTISSLQPEDFA | |
| | NSLRAEDTAVYYCARDGYGD | | TYYCQQSYSIPWTFGQGTKV | |
| | YPFDYWGQGTLVTVSS | | EIKR | |
| 29 | QVQLVQSGAEVKKPGASVKV | 306 | DIQMTQSPSSLSASVGDRVTI | 323 |
| | SCKASGYTFTGYYLHWVRQA | | TCRASQSISRWLAWYQQKPG | |
| | PGQGLEWMGVINVRRGSTRY | | KAPKLLIYDASNLETGVPSRF | |
| | AQNFQGRVTMTRDTSTSTVY | | SGSGSGTDFTLTISSLQPEDFA | |
| | MELSSLRSEDTAVYYCARVSG | | TYYCQQGNSFPPIFGGGTKVE | |
| | SYYQPWGQGTLVTVSS | | IKR | |
| 30 | QVQLVQSGAEVKKPGASVKV | 307 | DIQMTQSPSSLSASVGDRVTI | 324 |
| | SCKASGYTFSNYYMHWVRQA | | TCRASQSISSWLAWYQQKPG | |
| | PGQGLEWMGWMNPDSGTTG | | KAPKLLIYGASSLQSGVPSRF | |
| | YAQKFQGRVTMTRDTSTSTV | | SGSGSGTDFTLTISSLOPEDFA | |
| | YMELSSLRSEDTAVYYCVRD | | TYYCQQTYRTPLTFGPGTKV | |
| | GTMVQGIFDYWGQGTLVTVS | | DIKR | |
| | S | | | |
| 31 | QVQLVQSGAEVKKPGSSVKV | 308 | DIQMTQSPSSLSASVGDRVTI | 325 |
| | SCKASGGTFSTYAITWVRQAP | | TCRASQGIGNDLGWYQQKPG | |
| | GQGLEWMGGIIPIVGRANYAQ | | KAPKLLIYGASSVQSGVPSRF | |
| | KFQGRVTITADESTSTAYMEL | | SGSGSGTDFTLTISSLQPEDFA | |
| | SSLRSEDTAVYYCARSGGHDL | | TYYCQQSYSTPITFGQGTRLEI | |
| | DYWGQGTLVTVSS | | KR | |
| 32 | EVQLLESGGGLVQPGGSLRLS | 309 | EIVMTQSPATLSVSPGERATL | 326 |
| | CAASGFTFSSYGMHWVRQAP | | SCRASQSVSSSYLAWYQQKP | |
| | GKGLEWVSSISGSGDTTYYAD | | GQAPRLLIYATSTRATGIPAR | |
| | SVKGRFTISRDNSKNTLYLQM | | FSGSGSGTEFTLTISSLQSEDF | |
| | NSLRAEDTAVYYCARDNPYG | | AVYYCQQYGSLPLTFGQGTK | |
| | DYGGSFDYWGQGTLVTVSS | | VEIKR | |
| 33 | QVQLVQSGAEVKKPGASVKV | 310 | DIQMTQSPSSLSASVGDRVTI | 327 |
| | SCKASGYTFTSYYMHWVRQA | | TCRASQGISNNLNWYQQKPG | |
| | PGQGLEWMGIIDPSGGSTNYA | | KAPKLLIYDASNLETGVPSRF | |
| | QKFQGRVTMTRDTSTSTVYM | | SGSGSGTDFTLTISSLQPEDFA | |
| | ELSSLRSEDTAVYYCARDYYG | | TYYCQQANSFPLTFGPGTKV | |
| | SGSYYGLDYWGRGTLVTVSS | | DIKR | |
| 34 | QVQLVQSGAEVKKPGASVKV | 311 | DIQMTQSPSSLSASVGDRVTI | 328 |
| | SCKASGYTFTDYYMHWVRQA | | TCRASQGIRNDLAWYQQKPG | |
| | PGQGLEWMGIINPSGGSTRYA | | KAPKLLIYAASTLONGVPSRF | |
| | QKFQGRVTMTRDTSTSTVYM | | SGSGSGTDFTLTISSLQPEDFA | |
| | ELSSLRSEDTAVYYCARVDGR | | TYYCQQSYSTPWTFGQGTKV | |
| | RWLQSDYWGQGTLVTVSS | | EIKR | |
| 35 | QVQLVQSGAEVKKPGASVKV | 312 | DIQMTQSPSSLSASVGDRVTI | 329 |
| | SCKASGYTFTDYYMHWVRQA | | TCRASQGIRNDLAWYQQKPG | |
| | PGQGLEWMGIINPSGGSTRYA | | KAPKLLIYAASTLONGVPSRF | |
| | QKFQGRVTMTRDTSTSTVYM | | SGSGSGTDFTLTISSLQPEDFA | |
| | ELSSLRSEDTAVYYCARVDGR | | TYYCQQSYSTPWTFGQGTKV | |
| | RWLRSDYWGQGTLVTVSS | | EIKR | |
| 36 | QVQLVQSGAEVKKPGASVKV | 313 | DIQMTQSPSSLSASVGDRVTI | 330 |
| | SCKASGGTFSSYAISWVRQAP | | TCQASQGINNYLNWYQQKPG | |
| | GQGLEWLGIISPSGRSAGYGR | | KAPKLLIYAASTLQRGVPSRF | |
| | KFQGRVTMTRDTSTSTVYME | | SGSGSGTDFTLTISSLQPEDFA | |
| | LSSLRSEDTAVYYCARTDYGG | | TYYCQQSYQTPLTFGGGTKV | |
| | HKWYFDLWGRGTLVTVSS | | EIKR | |
| 37 | QVQLVQSGAEVKKPGASVKV | 314 | DIQMTQSPSSLSASVGDRVTI | 331 |
| | SCKASGYTFTGYYLHWVRQA | | TCRASQSISSYLNWYQQKPG | |
| | PGQGLEWMGVISPSGGGTSYA | | KAPKLLIYAASSLQSGVPSRF | |
| | QKFQGRVTMTRDTSTSTVYM | | SGSGSGTDFTLTISSLQPEDFA | |
| | ELSSLRSEDTAVYYCARAGFG | | TYYCQQSYSTPLTFGGGTKV | |
| | EGVFRHWGQGTLVTVSS | | EIKR | |
| 38 | QVQLVQSGAEVKKPGASVKV | 315 | DIQMTQSPSSLSASVGDRVTI | 332 |
| | SCKASGYSFTSHAISWVRQAP | | TCRASQGISNYLAWYQQKPG | |
| | GQGLEWMGWIKPNSGDTKYA | | KAPKLLIYTASTLQSGVPSRF | |
| | QKFQGRVTMTRDTSTSTVYM | | SGSGSGTDFTLTISSLQPEDFA | |
| | ELSSLRSEDTAVYYCARGSDD | | TYYCQQSYSTPLTFGGGTKV | |
| | YYGSYYFDYWGQGTLVTVSS | | EIKR | |
| 39 | EVQLLESGGGLVQPGGSLRLS | 316 | DIQMTQSPSSLSASVGDRVTI | 333 |
| | CAASGFTFRNYGMGWVRQAP | | TCRASQGISNDLAWYQQKPG | |
| | GKGLEWVSAISGSGGSTYYAD | | KAPKLLIYGASNLETGVPSRF | |
| | SVKGRFTISRDNSKNTLYLQM | | SGSGSGTDFTLTISSLQPEDFA | |
| | NSLRAEDTAVYYCARVKFYG | | TYYCQQANSFPFTFGPGTKV | |
| | MDVWGQGTTVTVSS | | DIKR | |
| 40 | QVQLVQSGAEVKKPGASVKV | 317 | DIQMTQSPSSLSASVGDRVTI | 334 |
| | SCKASGYTFTDYHMHWVRQA | | TCRVSQGISSYLNWYQQKPG | |
| | PGQGLEWMGWMSPNSGNTG | | KAPKLLIYEASTLESGVPSRFS | |
| | YAQNFQGRVTMTRDTSTSTV | | GSGSGTDFTLTISSLQPEDFAT | |
| | YMELSSLRSEDTAVYYCARA | | YYCQQGYSTPPTFGQGTKVEI | |
| | DYYGSDYVKFDYWGQGTLVT | | KR | |
| | VSS | | | |
| 41 | QVQLVQSGAEVKKPGASVKV | 318 | DIVMTQSPLSLPVTPGEPASIS | 335 |
| | SCKASGYTFPNYGISWVRQAP | | CRSSQSLLQSNGYNYLDWYL | |
| | GQGLEWMGWINPNSGGTKYA | | QKPGQSPQLLIYLGSNRASGV | |
| | QRFQGRVTMTRDTSTSTVYM | | PDRFSGSGSGTDFTLKISRVE | |
| | ELSSLRSEDTAVYYCARDRDI | | AEDVGVYYCMQSTHWPLTF | |
| | LTGYYHFDYWGQGTLVTVSS | | GQGTRLEIKR | |
| 42 | QVQLVQSGAEVKKPGASVKV | 319 | DIQMTQSPSSLSASVGDRVTI | 336 |
| | SCKASGYTFTDYFMHWVRQA | | TCRASQGISNNLNWYQQKPG | |
| | PGQGLEWMGWINPNSGNTGY | | KAPKLLIYAASSLQSGVPSRF | |
| | AQKFQGRVTMTRDTSTSTVY | | SGSGSGTDFTLTISSLQPEDFA | |
| | MELSSLRSEDTAVYYCARLND | | TYYCQQSYSTPPTFGQGTKLE | |
| | YGDYGGPATLDYWGQGTLVT | | IKR | |
| | VSS | | | |

### Example 3: Identification of Anti-Human CLL-1 scFv Clones

Methods analogous to those above in Example 1 have been used to discover selective anti-human CLL-1 scFv clones. Selective anti-human CLL-1 scFv clones were discovered by standard screening methodologies of a human antibody library using two recombinant polymorphic forms of human CLL-1 extracellular domain antigens (CLL-1-K244 and CLL-1-Q244). Using these antigens various panning tactics were employed to encourage enrichment of thermostable clones of desired affinity range. The scFvs were screened for selective binding between two single nucleotide polymorphism (SNP) variants of human CLL-1 (Lysine 244 and Glutamine 244) by bio-layer interferometry (BLI).

TABLE 6a
Sequences of Anti-CLL-1 K244 Selective Polypeptides (CDR Sequences)

| Polypep- | | SEQ ID | | SEQ ID | | SEQ ID | | SEQ ID | | SEQ ID | | SEQ ID |
|---|---|---|---|---|---|---|---|---|---|---|---|---|
| tide No. | HCDR1 | NO | HCDR2 | NO | HCDR3 | NO | LCDR1 | NO | LCDR2 | NO | LCDR3 | NO |
| 43 | YTFTNY | 337 | GWISPY | 361 | CARES | 385 | RASQSI | 409 | DASNLE | 433 | CQQSYS | 457 |
| | YMH | | SGDTK | | MDRLD | | STYLN | | T | | TPVLTF | |
| | | | YA | | YW | | | | | | | |
| 44 | FTFSSY | 338 | ADISGS | 362 | CAREG | 386 | RSSQSL | 410 | LGSNRA | 434 | CMQAL | 458 |
| | AMH | | GGLTY | | DQYSSS | | LHSNG | | S | | QPPPTF | |
| | | | YA | | SFFDY | | YNYLD | | | | | |
| | | | | | W | | | | | | | |
| 45 | FTFDEF | 339 | SYISGD | 363 | CAAGY | 387 | QASQDI | 411 | AASTLE | 435 | CQQSYS | 459 |
| | GMN | | SGYTNC | | GGYYF | | DIYLN | | S | | TPPTF | |
| | | | A | | DYW | | | | | | | |
| 46 | YTFTSY | 340 | GMINPS | 364 | CASVDS | 388 | RASQSI | 412 | DASNLE | 436 | CQQAN | 460 |
| | YMH | | AGSTSY | | SGWYA | | STYLN | | T | | SFPPTF | |
| | | | A | | PFDYW | | | | | | | |
| 47 | FTFDEY | 341 | SAIGAG | 365 | CASSLG | 389 | RSSQSL | 413 | AASSLQ | 437 | CMQGI | 461 |
| | AMH | | GSTYY | | PELRGV | | LHSNG | | S | | QWPWT | |
| | | | A | | DYYYY | | YNYLD | | | | F | |
| | | | | | GMDVW | | | | | | | |
| 48 | FNFDDY | 342 | SVIYSG | 366 | CTRHDF | 390 | RASQSI | 414 | AASSLQ | 438 | CQQDY | 462 |
| | AMH | | GSTYY | | DYW | | STYVN | | S | | SYPYTF | |
| | | | A | | | | | | | | | |
| 49 | FTFSDY | 343 | SLISGD | 367 | CARDL | 391 | RASQSI | 415 | AASTLQ | 439 | CLQDYS | 463 |
| | ALH | | GGSTY | | GGERSY | | STWLA | | S | | YPPTF | |
| | | | YA | | W | | | | | | | |
| 50 | YTFTDY | 344 | GIINPSD | 368 | CARDEL | 392 | RASQSI | 416 | AASSLQ | 440 | CQQSY | 464 |
| | YMH | | GSTTYA | | PDSSG | | SSWLA | | S | | DIPLTF | |
| | | | | | WYGYF | | | | | | | |
| | | | | | QHW | | | | | | | |
| 51 | GTFSSY | 345 | GEIIPFF | 369 | CARAE | 393 | QASQDI | 417 | AASTLQ | 441 | CQQSY | 465 |
| | AIS | | GTANY | | YGGDL | | SNLLN | | S | | NTPWTF | |
| | | | A | | DYW | | | | | | | |
| 52 | DTFTRH | 346 | GIINPR | 370 | CARRD | 394 | QASQDI | 418 | QASSLE | 442 | CQQAN | 466 |
| | YVH | | GGTHY | | CSGGSC | | HNYLN | | S | | SFPLTF | |
| | | | A | | YSDLD | | | | | | | |
| | | | | | YW | | | | | | | |
| 53 | GTFSSY | 347 | GWINPD | 371 | CATFGE | 395 | RASQNI | 419 | GASILQ | 443 | CQQAN | 467 |
| | AIS | | SGDASY | | EAFDIW | | GSWLA | | S | | SFPLTF | |
| | | | A | | | | | | | | | |
| 54 | GTFSSY | 348 | GWIDPK | 372 | CATEGS | 396 | RASQGI | 420 | EASTLQ | 444 | CHQYN | 468 |
| | AIS | | NGDTN | | HHPYY | | GNWLA | | S | | AYPWT | |
| | | | YA | | YYGMD | | | | | | F | |
| | | | | | VW | | | | | | | |
| 55 | YTFTGY | 349 | GWINPN | 373 | CARPNT | 397 | QASQDI | 421 | AASSLQ | 445 | CQQYN | 469 |
| | HMH | | TGGTN | | AMVPP | | SNYLN | | S | | SYPLTF | |
| | | | YA | | YYYYY | | | | | | | |
| | | | | | GMDVW | | | | | | | |
| 56 | YTFTSY | 350 | GWMNP | 374 | CARVS | 398 | RASHSI | 422 | DASNLE | 446 | CQQAD | 470 |
| | DIN | | NSGNT | | ATGTY | | SSWLA | | T | | SFPLTF | |
| | | | GYA | | GLDYW | | | | | | | |
| 57 | YTFNN | 351 | GIINPIT | 375 | CASGEQ | 399 | QASQDI | 423 | GASNL | 447 | CLQHNS | 471 |
| | YGIT | | GVTTY | | QLVLFD | | NDYLN | | QS | | YPLTF | |
| | | | A | | YW | | | | | | | |
| 58 | YTFTDY | 352 | GWMNP | 376 | CAADVI | 400 | RASQGI | 424 | DASNLE | 448 | CQQSY | 472 |
| | YLH | | NSGNT | | TAYGM | | SNYLA | | T | | NVPPTF | |
| | | | GYA | | DVW | | | | | | | |
| 59 | FTFSNA | 353 | ADISYD | 377 | CTTEEL | 401 | RASQSI | 425 | DASNLE | 449 | CQQAN | 473 |
| | WMS | | GTNDY | | RFGGFD | | SSYLN | | T | | SFPLTF | |
| | | | YA | | YW | | | | | | | |
| 60 | GTFSSY | 354 | GGIIPM | 378 | CARDL | 402 | RASQSI | 426 | DASSRA | 450 | CQQYK | 474 |
| | AIS | | FGTAN | | GYSNA | | GTYLA | | T | | SYPLTF | |
| | | | YA | | GGTLH | | | | | | | |
| | | | | | YW | | | | | | | |
| 61 | YTFTNY | 355 | GIINPSG | 379 | CARAE | 403 | QASQDI | 427 | GASSLQ | 451 | CQQHN | 475 |
| | YMH | | GSTSYA | | WDILTG | | SNYLN | | S | | SYPWTF | |
| | | | | | YYIDY | | | | | | | |
| | | | | | W | | | | | | | |
| 62 | YTFTDH | 356 | GWISAY | 380 | CARAE | 404 | RASQGI | 428 | DASNLE | 452 | CQQTSS | 476 |
| | FVH | | NGNTN | | YSYGFD | | HNYLA | | T | | FPYTF | |
| | | | YA | | YW | | | | | | | |
| 63 | YTFTGY | 357 | GVINPS | 381 | CARDRS | 405 | QASQDI | 429 | DASNL | 453 | CLQHNS | 477 |
| | YVH | | GGGSPS | | DVDYG | | SNYLN | | QS | | YPLTF | |
| | | | YA | | MDVW | | | | | | | |
| 64 | YTFTDY | 358 | GLIDPS | 382 | CARDV | 406 | RSSQSL | 430 | AASTLQ | 454 | CMQGT | 478 |
| | YMH | | GGSTNS | | GFGELS | | LHSNG | | S | | HWPPTF | |
| | | | L | | FDIW | | YNYLD | | | | | |
| 65 | YTFTGY | 359 | GWINPN | 383 | CAREIG | 407 | RASQSI | 431 | AASSLQ | 455 | CQQSYT | 479 |
| | YMH | | SGGTN | | GYDNY | | GTYLN | | S | | DPWTF | |
| | | | YA | | YYYGM | | | | | | | |
| | | | | | DVW | | | | | | | |
| 66 | YTFNTY | 360 | GWMHP | 384 | CARGTT | 408 | RASQSI | 432 | SASNLQ | 456 | CQQSYS | 480 |
| | YMH | | NTGNT | | SDAFDI | | FSYLN | | S | | TPITF | |
| | | | GYA | | W | | | | | | | |

TABLE 6b
Sequences of Anti-CLL-1 K244 Selective Polypeptides (VH and VL Sequences)

| Polypep- | | SEQ ID | | SEQ ID |
|---|---|---|---|---|
| tide No. | Full VH | NO | Full VL | NO |
| 43 | QVQLVQSGAEVKKPGASVKV | 481 | DIQMTQSPSSLSASVGDRVTI | 505 |
| | SCKASGYTFTNYYMHWVRQA | | TCRASQSISTYLNWYQQKPG | |
| | PGQGLEWLGWISPYSGDTKY | | KAPKLLIYDASNLETGVPSRF | |
| | AQTLQGRVTMTRDTSTSTVY | | SGSGSGTDFTLTISSLQPEDFA | |
| | MELSSLRSEDTAVYYCARESM | | TYYCQQSYSTPVLTFGGGTK | |
| | DRLDYWGQGTLVTVSS | | VEIKR | |
| 44 | EVQLLESGGGLVQPGGSLRLS | 482 | DIVMTQSPLSLPVTPGEPASIS | 506 |
| | CAASGFTFSSYAMHWVRQAP | | CRSSQSLLHSNGYNYLDWYL | |
| | GKGLEWVADISGSGGLTYYA | | QKPGQSPQLLIYLGSNRASGV | |
| | DSVKGRFTISRDNSKNTLYLQ | | PDRFSGSGSGTDFTLKISRVE | |
| | MNSLRAEDTAVYYCAREGDQ | | AEDVGVYYCMQALQPPPTFG | |
| | YSSSSFFDYWGQGTLVTVSS | | QGTRLEIKR | |
| 45 | EVQLVESGGGLVKPGGSLRLS | 483 | DIQMTQSPSSLSASVGDRVTI | 507 |
| | CAASGFTFDEFGMNWVRQAP | | TCQASQDIDIYLNWYQQKPG | |
| | GKGLEWISYISGDSGYTNCAD | | KAPKLLIYAASTLESGVPSRF | |
| | SVKGRFTISRDDSKNTLYLQM | | SGSGSGTDFTLTISSLQPEDFA | |
| | NSLKTEDTAVYYCAAGYGGY | | TYYCQQSYSTPPTFGGGTKV | |
| | YFDYWGQGTLVTVSS | | EIKR | |
| 46 | QVQLVQSGAEVKKPGASVKV | 484 | DIQMTQSPSSLSASVGDRVTI | 508 |
| | SCKASGYTFTSYYMHWVRQA | | TCRASQSISTYLNWYQQKPG | |
| | PGQGLEWMGMINPSAGSTSY | | KAPKLLIYDASNLETGVPSRF | |
| | AQKFQGRVTMTRDTSTSTVY | | SGSGSGTDFTLTISSLQPEDFA | |
| | MELSSLRSEDTAVYYCASVDS | | TYYCQQANSFPPTFGGGTKV | |
| | SGWYAPFDYWGQGTLVTVSS | | EIKR | |
| 47 | EVQLLESGGGLVQPGGSLRLS | 485 | DIVMTQSPLSLPVTPGEPASIS | 509 |
| | CAASGFTFDEYAMHWVRQAP | | CRSSQSLLHSNGYNYLDWYL | |
| | GKGLEWVSAIGAGGSTYYAD | | QKPGQSPQLLIYAASSLQSGV | |
| | SVKGRFTISRDNSKNTLYLQM | | PDRFSGSGSGTDFTLKISRVE | |
| | NSLRAEDTAVYYCASSLGPEL | | AEDVGVYYCMQGIQWPWTF | |
| | RGVDYYYYGMDVWGQGTTV | | GQGTKVEIKR | |
| | TVSS | | | |
| 48 | EVQLLESGGGLVQPGGSLRLS | 486 | DIQMTQSPSSLSASVGDRVTI | 510 |
| | CAASGFNFDDYAMHWVRQA | | TCRASQSISTYVNWYQQKPG | |
| | PGKGLEWVSVIYSGGSTYYAD | | KAPKLLIYAASSLQSGVPSRF | |
| | SVKGRFTISRDNSKNTLYLQM | | SGSGSGTDFTLTISSLQPEDFA | |
| | NSLRAEDTAVYYCTRHDFDY | | TYYCQQDYSYPYTFGQGTKV | |
| | WGQGTLVTVSS | | EIKR | |
| 49 | EVOLVESGGGLVKPGGSLRLS | 487 | DIQMTQSPSSLSASVGDRVTI | 511 |
| | CAASGFTFSDYALHWVRQAP | | TCRASQSISTWLAWYQQKPG | |
| | GKGLEWVSLISGDGGSTYYA | | KAPKLLIYAASTLQSGVPSRF | |
| | DSVKGRFTISRDDSKNTLYLQ | | SGSGSGTDFTLTISSLQPEDFA | |
| | MNSLKTEDTAVYYCARDLGG | | TYYCLQDYSYPPTFGQGTKV | |
| | ERSYWGQGTLVTVSS | | EIKR | |
| 50 | QVQLVQSGAEVKKPGASVKV | 488 | DIQMTQSPSSLSASVGDRVTI | 512 |
| | SCKASGYTFTDYYMHWVRQA | | TCRASQSISSWLAWYQQKPG | |
| | PGQGLEWMGIINPSDGSTTYA | | KAPKLLIYAASSLQSGVPSRF | |
| | QSFQGRVTMTRDTSTSTVYM | | SGSGSGTDFTLTISSLOPEDFA | |
| | ELSSLRSEDTAVYYCARDELP | | TYYCQQSYDIPLTFGGGTKVE | |
| | DSSGWYGYFQHWGQGTLVT | | IKR | |
| | VSS | | | |
| 51 | QVQLVQSGAEVKKPGSSVKV | 489 | DIQMTQSPSSLSASVGDRVTI | 513 |
| | SCKASGGTFSSYAISWVRQAP | | TCQASQDISNLLNWYQQKPG | |
| | GOGLEWMGEIIPFFGTANYAQ | | KAPKLLIYAASTLQSGVPSRF | |
| | KFQGRVTITADESTSTAYMEL | | SGSGSGTDFTLTISSLQPEDFA | |
| | SSLRSEDTAVYYCARAEYGG | | TYYCQQSYNTPWTFGPGTKV | |
| | DLDYWGQGTLVTVSS | | DIKR | |
| 52 | QVQLVQSGAEVKKPGASVKV | 490 | DIQMTQSPSSLSASVGDRVTI | 514 |
| | SCKASGDTFTRHYVHWVRQA | | TCQASQDIHNYLNWYQQKPG | |
| | PGQGLEWMGIINPRGGTHYA | | KAPKLLIYQASSLESGVPSRFS | |
| | QKFQGRVTMTRDTSTSTVYM | | GSGSGTDFTLTISSLQPEDFAT | |
| | ELSSLRSEDTAVYYCARRDCS | | YYCQQANSFPLTFGGGTKLEI | |
| | GGSCYSDLDYWGQGTLVTVS | | KR | |
| | S | | | |
| 53 | QVQLVQSGAEVKKPGASVKV | 491 | DIQMTQSPSSLSASVGDRVTI | 515 |
| | SCKASGGTFSSYAISWVRQAP | | TCRASQNIGSWLAWYQQKPG | |
| | GQGLEWMGWINPDSGDASYA | | KAPKLLIYGASILQSGVPSRFS | |
| | RKFQGRVTMTRDTSTSTVYM | | GSGSGTDFTLTISSLQPEDFAT | |
| | ELSSLRSEDTAVYYCATFGEE | | YYCQQANSFPLTFGGGTKLEI | |
| | AFDIWGQGTMVTVSS | | KR | |
| 54 | QVQLVQSGAEVKKPGASVKV | 492 | DIQMTQSPSSLSASVGDRVTI | 516 |
| | SCKASGGTFSSYAISWVRQAP | | TCRASQGIGNWLAWYQQKP | |
| | GQGLEWMGWIDPKNGDTNY | | GKAPKLLIYEASTLQSGVPSR | |
| | AQKFQGRVTMTRDTSTSTVY | | FSGSGSGTDFTLTISSLQPEDF | |
| | MELSSLRSEDTAVYYCATEGS | | ATYYCHQYNAYPWTFGQGT | |
| | HHPYYYYGMDVWGQGTTVT | | KVEIKR | |
| | VSS | | | |
| 55 | QVQLVQSGAEVKKPGASVKV | 493 | DIQMTQSPSSLSASVGDRVTI | 517 |
| | SCKASGYTFTGYHMHWVRQA | | TCQASQDISNYLNWYQQKPG | |
| | PGQGLEWMGWINPNTGGTNY | | KAPKLLIYAASSLQSGVPSRF | |
| | AQKFQGRVTMTRDTSTSTVY | | SGSGSGTDFTLTISSLQPEDFA | |
| | MELSSLRSEDTAVYYCARPNT | | TYYCQQYNSYPLTFGQGTKL | |
| | AMVPPYYYYYGMDVWGQGT | | EIKR | |
| | LVTVSS | | | |
| 56 | QVQLVQSGAEVKKPGASVKV | 494 | DIQMTQSPSSLSASVGDRVTI | 518 |
| | SCKASGYTFTSYDINWVRQAP | | TCRASHSISSWLAWYQQKPG | |
| | GQGLEWMGWMNPNSGNTGY | | KAPKLLIYDASNLETGVPSRF | |
| | AQKFQGRVTMTRDTSTSTVY | | SGSGSGTDFTLTISSLOPEDFA | |
| | MELSSLRSEDTAVYYCARVSA | | TYYCQQADSFPLTFGGGTKV | |
| | TGTYGLDYWGQGTLVTVSS | | EIKR | |
| 57 | QVQLVQSGAEVKKPGASVKV | 495 | DIQMTQSPSSLSASVGDRVTI | 519 |
| | SCKASGYTFNNYGITWVRQAP | | TCQASQDINDYLNWYQQKPG | |
| | GQGLEWMGIINPITGVTTYAQ | | KAPKLLIYGASNLQSGVPSRF | |
| | NFQGRVTMTRDTSTSTVYME | | SGSGSGTDFTLTISSLQPEDFA | |
| | LSSLRSEDTAVYYCASGEQQL | | TYYCLQHNSYPLTFGQGTKL | |
| | VLFDYWGQGTLVTVSS | | EIKR | |
| 58 | QVQLVQSGAEVKKPGASVKV | 496 | DIQMTQSPSSLSASVGDRVTI | 520 |
| | SCKASGYTFTDYYLHWVRQA | | TCRASQGISNYLAWYQQKPG | |
| | PGQGLEWMGWMNPNSGNTG | | KAPKLLIYDASNLETGVPSRF | |
| | YAQKFQGRVTMTRDTSTSTV | | SGSGSGTDFTLTISSLQPEDFA | |
| | YMELSSLRSEDTAVYYCAAD | | TYYCQQSYNVPPTFGQGTKV | |
| | VITAYGMDVWGQGTMVTVSS | | EIKR | |
| 59 | EVQLLESGGGLVQPGGSLRLS | 497 | DIQMTQSPSSLSASVGDRVTI | 521 |
| | CAASGFTFSNAWMSWVRQAP | | TCRASQSISSYLNWYQQKPG | |
| | GKGLEWVADISYDGTNDYYA | | KAPKLLIYDASNLETGVPSRF | |
| | DSVKGRFTISRDNSKNTLYLQ | | SGSGSGTDFTLTISSLQPEDFA | |
| | MNSLRAEDTAVYYCTTEELRF | | TYYCQQANSFPLTFGQGTKV | |
| | GGFDYWGQGTLVTVSS | | EIKR | |
| 60 | QVQLVQSGAEVKKPGSSVKV | 498 | EIVMTQSPATLSVSPGERATL | 522 |
| | SCKASGGTFSSYAISWVRQAP | | SCRASQSIGTYLAWYQQKPG | |
| | GQGLEWMGGIIPMFGTANYA | | QAPRLLIYDASSRATGIPARFS | |
| | QKFQGRVTITADESTSTAYME | | GSGSGTEFTLTISSLQSEDFAV | |
| | LSSLRSEDTAVYYCARDLGYS | | YYCQQYKSYPLTFGGGTKVE | |
| | NAGGTLHYWGQGTLVTVSS | | IKR | |
| 61 | QVQLVQSGAEVKKPGASVKV | 499 | DIQMTQSPSSLSASVGDRVTI | 523 |
| | SCKASGYTFTNYYMHWVRQA | | TCQASQDISNYLNWYQQKPG | |
| | PGQGLEWMGIINPSGGSTSYA | | KAPKLLIYGASSLQSGVPSRF | |
| | QKFQGRVTMTRDTSTSTVYM | | SGSGSGTDFTLTISSLQPEDFA | |
| | ELSSLRSEDTAVYYCARAEW | | TYYCQQHNSYPWTFGQGTK | |
| | DILTGYYIDYWGQGTLVTVSS | | VEIKR | |
| 62 | QVQLVQSGAEVKKPGASVKV | 500 | DIQMTQSPSSLSASVGDRVTI | 524 |
| | SCKASGYTFTDHFVHWVRQA | | TCRASQGIHNYLAWYQQKPG | |
| | PGQGLEWMGWISAYNGNTNY | | KAPKLLIYDASNLETGVPSRF | |
| | AQKFQGRVTMTRDTSTSTVY | | SGSGSGTDFTLTISSLQPEDFA | |
| | MELSSLRSEDTAVYYCARAEY | | TYYCQQTSSFPYTFGQGTKLE | |
| | SYGFDYWGQGTLVTVSS | | IKR | |
| 63 | QVQLVQSGAEVKKPGASVKV | 501 | DIQMTQSPSSLSASVGDRVTI | 525 |
| | SCKASGYTFTGYYVHWVRQA | | TCQASQDISNYLNWYQQKPG | |
| | PGQGLEWMGVINPSGGGSPSY | | KAPKLLIYDASNLQSGVPSRF | |
| | AQKFQGRVTMTRDTSTSTVY | | SGSGSGTDFTLTISSLOPEDFA | |
| | MELSSLRSEDTAVYYCARDRS | | TYYCLQHNSYPLTFGGGTKV | |
| | DVDYGMDVWGQGTTVTVSS | | EIKR | |
| 64 | QVQLVQSGAEVKKPGASVKV | 502 | DIVMTQSPLSLPVTPGEPASIS | 526 |
| | SCKASGYTFTDYYMHWVRQA | | CRSSQSLLHSNGYNYLDWYL | |
| | PGQGLEWMGLIDPSGGSTNSL | | QKPGQSPQLLIYAASTLQSGV | |
| | QKFQGRVTMTRDTSTSTVYM | | PDRFSGSGSGTDFTLKISRVE | |
| | ELSSLRSEDTAVYYCARDVGF | | AEDVGVYYCMQGTHWPPTF | |
| | GELSFDIWGQGTTVTVSS | | GPGTKVDIKR | |
| 65 | QVQLVQSGAEVKKPGASVKV | 503 | DIQMTQSPSSLSASVGDRVTI | 527 |
| | SCKASGYTFTGYYMHWVRQA | | TCRASQSIGTYLNWYQQKPG | |
| | PGQGLEWMGWINPNSGGTNY | | KAPKLLIYAASSLQSGVPSRF | |
| | AQKFQGRVTMTRDTSTSTVY | | SGSGSGTDFTLTISSLOPEDFA | |
| | MELSSLRSEDTAVYYCAREIG | | TYYCQQSYTDPWTFGQGTKV | |
| | GYDNYYYYGMDVWGQGTTV | | EIKR | |
| | TVSS | | | |
| 66 | QVQLVQSGAEVKKPGASVKV | 504 | DIQMTQSPSSLSASVGDRVTI | 528 |
| | SCKASGYTFNTYYMHWVRQA | | TCRASQSIFSYLNWYQQKPG | |
| | PGQGLEWMGWMHPNTGNTG | | KAPKLLIYSASNLQSGVPSRF | |
| | YAQKFQGRVTMTRDTSTSTV | | SGSGSGTDFTLTISSLQPEDFA | |
| | YMELSSLRSEDTAVYYCARGT | | TYYCQQSYSTPITFGQGTKVE | |
| | TSDAFDIWGQGTMVTVSS | | IKR | |

TABLE 7a
Sequences of Anti-CLL-1 K244Q Selective Polypeptides (CDR Sequences)

| Polypeptide | | SEQ ID | | SEQ ID | | SEQ ID | | SEQ ID | | SEQ ID | | SEQ ID |
|---|---|---|---|---|---|---|---|---|---|---|---|---|
| No. | HCDR1 | NO | HCDR2 | NO | HCDR3 | NO | LCDR1 | NO | LCDR2 | NO | LCDR3 | NO |
| 67 | DTFTR | 529 | GRVNP | 551 | CAKD | 573 | RASQG | 595 | DASNL | 617 | CQQAS | 639 |
| | HYVH | | RDGRT | | MFPTV | | ISSYLA | | ET | | GFPYT | |
| | | | NSA | | TGTYY | | | | | | F | |
| | | | | | YYGM | | | | | | | |
| | | | | | DVW | | | | | | | |
| 68 | YTFSS | 530 | GWINP | 552 | CARHR | 574 | RASQSI | 596 | ATSSL | 618 | CQQGY | 640 |
| | YDIN | | RNGGT | | WELDS | | SNYLN | | QS | | NIPFTF | |
| | | | DYA | | FDYW | | | | | | | |
| 69 | YTFTS | 531 | GWMN | 553 | CARDD | 575 | RASESI | 597 | DASNL | 619 | CQQYD | 641 |
| | YYIH | | PNDGK | | DYGGY | | SGWLA | | ET | | TWPFT | |
| | | | TAYA | | VAYW | | | | | | F | |
| 70 | MSVTS | 532 | SSIYPD | 554 | CARDE | 576 | QASQSI | 598 | AASTL | 620 | CQQSY | 642 |
| | NHMS | | GKTYY | | EDWFD | | SNWLA | | QS | | STPWT | |
| | | | A | | PW | | | | | | F | |
| 71 | FTFSN | 533 | AVIWP | 555 | CARED | 577 | QASQD | 599 | GASTL | 621 | CQQYD | 643 |
| | HYMS | | DGSKE | | YYGSG | | ISNYL | | QS | | SYPPTF | |
| | | | YYA | | MDYW | | N | | | | | |
| 72 | GTFSN | 534 | GWISA | 556 | CAIGD | 578 | QASED | 600 | DASNL | 622 | CQQAN | 644 |
| | YAIS | | YNGNS | | YFDY | | INKYL | | ET | | SFPLTF | |
| | | | DYA | | W | | N | | | | | |
| 73 | FTVSS | 535 | AVIYS | 557 | CARED | 579 | RASQSI | 601 | DASNL | 623 | CQQAH | 645 |
| | NYMS | | DGKTY | | SSGSH | | STYLN | | ET | | SFPPTF | |
| | | | YA | | FDYW | | | | | | | |
| 74 | YTFTK | 536 | GGIIPIF | 558 | CARGS | 580 | RASQG | 602 | DASYL | 624 | CQQSY | 646 |
| | YEIN | | GTANY | | GWYTP | | ISNNL | | ET | | SAPLTF | |
| | | | A | | LFDYW | | N | | | | | |
| 75 | YTFTD | 537 | GLIDPS | 559 | CARDY | 581 | RASQS | 603 | DASAR | 625 | CQQYR | 647 |
| | YYIH | | GGSTSI | | DILTGS | | VSSYL | | AT | | SSVTF | |
| | | | A | | GFDPW | | A | | | | | |
| 76 | YTFTT | 538 | GIINVS | 560 | CAKEP | 582 | QASQD | 604 | DASNL | 626 | CQQAN | 648 |
| | YYMH | | AGTTS | | YPHQS | | INNYL | | ET | | SFPLTF | |
| | | | YA | | GWFFD | | N | | | | | |
| | | | | | YW | | | | | | | |
| 77 | YTFTG | 539 | GWIST | 561 | CARDT | 583 | SASQS | 605 | DVSTR | 627 | CQQYY | 649 |
| | HYMH | | DNGNA | | ADYYF | | VGSSY | | AT | | STPLTF | |
| | | | NYA | | DYW | | FA | | | | | |
| 78 | GTFSR | 540 | GWMN | 562 | CARGD | 584 | QASQD | 606 | DASNL | 628 | CQQSY | 650 |
| | YPFS | | PNNGD | | YPYMD | | ISNYL | | ET | | SIPYTF | |
| | | | TGYA | | VW | | N | | | | | |
| 79 | YTFTS | 541 | GWMN | 563 | CARDY | 585 | RASQG | 607 | AASSL | 629 | CLQTN | 651 |
| | DYMH | | PNSGG | | ITGPSD | | IRNDL | | QP | | SFPWT | |
| | | | TNYA | | W | | G | | | | F | |
| 80 | FTFTSY | 542 | GWMN | 564 | CARGH | 586 | RASQSI | 608 | DTSSL | 630 | CQQGY | 652 |
| | YMH | | PNSGN | | SRTDY | | SSWLA | | QS | | STPLTF | |
| | | | TGYA | | GMDV | | | | | | | |
| | | | | | W | | | | | | | |
| 81 | FTFSD | 543 | SIIYPD | 565 | CAREG | 587 | QASQD | 609 | GASTL | 631 | CQQSY | 653 |
| | HYMS | | GKTYY | | SYGDY | | ISNYL | | QS | | STPWT | |
| | | | A | | DGMD | | N | | | | F | |
| | | | | | VW | | | | | | | |
| 82 | GTFSN | 544 | GGIIPIF | 566 | CAREA | 588 | RASQS | 610 | GASTR | 632 | CQQYA | 654 |
| | YDIS | | GTANY | | EEGGW | | VSSYL | | AT | | FSPITF | |
| | | | A | | FDPW | | A | | | | | |
| 83 | YTFTD | 545 | GWMN | 567 | CAKDT | 589 | RVSQG | 611 | DASNL | 633 | CQQSY | 655 |
| | YYMH | | PNSGY | | PGSGW | | ISSYLN | | ET | | STPLTF | |
| | | | TAYA | | SSGMD | | | | | | | |
| | | | | | VW | | | | | | | |
| 84 | GTFSN | 546 | GWINP | 568 | CARVG | 590 | RASQSI | 612 | DASNL | 634 | CLQTH | 656 |
| | YAIS | | NSGGT | | YYDSS | | SSWLA | | ET | | SFPLTF | |
| | | | NYA | | GGGM | | | | | | | |
| | | | | | DVW | | | | | | | |
| 85 | YTFTG | 547 | GIINPI | 569 | CASGA | 591 | RASQS | 613 | DASNL | 635 | CQQAN | 657 |
| | YYMH | | GGLTT | | YGDYV | | VSNWL | | QT | | SFPLTF | |
| | | | YA | | DWYF | | A | | | | | |
| | | | | | DLW | | | | | | | |
| 86 | YTFTT | 548 | GWINP | 570 | CARLT | 592 | RSSRSL | 614 | LGSYR | 636 | CMQGT | 658 |
| | YGIS | | NSGDT | | TATDS | | LHSNG | | AS | | HWPPT | |
| | | | NYA | | FDLW | | YNYLD | | | | F | |
| 87 | YSFTN | 549 | GWMN | 571 | CTTDE | 593 | RASQSI | 615 | DASNL | 637 | CQQAN | 659 |
| | YYIH | | PYTGQ | | ETMDF | | SRYLN | | ET | | TFPITF | |
| | | | TGYA | | HLW | | | | | | | |
| 88 | YTFTG | 550 | GRINP | 572 | CARET | 594 | RSSRSL | 616 | LGSDR | 638 | CMQGT | 660 |
| | YHIH | | NSGGT | | YSGSY | | LHSNG | | AS | | HWPPT | |
| | | | DYA | | EESFD | | YNYLD | | | | F | |
| | | | | | YW | | | | | | | |

TABLE 7b
Sequences of Anti-CLL-1 K244Q Polypeptides (VH and VL Sequences)

| | | SEQ ID | | SEQ ID |
|---|---|---|---|---|
| Polypeptide No. | Full VH | NO | Full VL | NO |
| 67 | QVQLVQSGAEVKKPGASVKV | 661 | DIQMTQSPSSLSASVGDRVTI | 683 |
| | SCKASGDTFTRHYVHWVRQA | | TCRASQGISSYLAWYQQKPG | |
| | PGQGLEWMGRVNPRDGRTNS | | KAPKLLIYDASNLETGVPSRF | |
| | AQKFQGRVTMTRDTSTSTVY | | SGSGSGTDFTLTISSLQPEDFA | |
| | MELSSLRSEDTAVYYCAKDM | | TYYCQQASGFPYTFGQGTRL | |
| | FPTVTGTYYYYGMDVWGQG | | EIKR | |
| | TTVTVSS | | | |
| 68 | QVQLVQSGAEVKKPGASVKV | 662 | DIQMTQSPSSLSASVGDRVTI | 684 |
| | SCKASGYTFSSYDINWVRQAP | | TCRASQSISNYLNWYQQKPG | |
| | GQGLEWVGWINPRNGGTDYA | | KAPKLLIYATSSLQSGVPSRFS | |
| | QKFQGRVTMTRDTSTSTVYM | | GSGSGTDFTLTISSLQPEDFAT | |
| | ELSSLRSEDTAVYYCARHRWE | | YYCQQGYNIPFTFGQGTKLEI | |
| | LDSFDYWGQGTLVTVSS | | KR | |
| 69 | QVQLVQSGAEVKKPGASVKV | 663 | DIQMTQSPSSLSASVGDRVTI | 685 |
| | SCKASGYTFTSYYIHWVRQAP | | TCRASESISGWLAWYQQKPG | |
| | GQGLEWMGWMNPNDGKTAY | | KAPKLLIYDASNLETGVPSRF | |
| | AQRFQGRVTMTRDTSTSTVY | | SGSGSGTDFTLTISSLQPEDFA | |
| | MELSSLRSEDTAVYYCARDD | | TYYCQQYDTWPFTFGPGTKV | |
| | DYGGYVAYWGQGTLVTVSS | | DIKR | |
| 70 | EVQLLESGGGLVQPGGSLRLS | 664 | DIQMTQSPSSLSASVGDRVTI | 686 |
| | CAASGMSVTSNHMSWVRQAP | | TCQASQSISNWLAWYQQKPG | |
| | GKGLEWVSSIYPDGKTYYADS | | KAPKLLIYAASTLQSGVPSRF | |
| | VKGRFTISRDNSKNTLYLQMN | | SGSGSGTDFTLTISSLQPEDFA | |
| | SLRAEDTAVYYCARDEEDWF | | TYYCQQSYSTPWTFGQGTKV | |
| | DPWGQGTLVTVSS | | EIKR | |
| 71 | EVQLLESGGGLVQPGGSLRLS | 665 | DIQMTQSPSSLSASVGDRVTI | 687 |
| | CAASGFTFSNHYMSWVRQAP | | TCQASQDISNYLNWYQQKPG | |
| | GKGLEWVAVIWPDGSKEYYA | | KAPKLLIYGASTLQSGVPSRF | |
| | DSVKGRFTISRDNSKNTLYLQ | | SGSGSGTDFTLTISSLQPEDFA | |
| | MNSLRAEDTAVYYCAREDYY | | TYYCQQYDSYPPTFGGGTKV | |
| | GSGMDYWGQGTLVTVSS | | EIKR | |
| 72 | QVQLVQSGAEVKKPGASVKV | 666 | DIQMTQSPSSLSASVGDRVTI | 688 |
| | SCKASGGTFSNYAISWVRQAP | | TCQASEDINKYLNWYQQKPG | |
| | GQGLEWMGWISAYNGNSDY | | KAPKLLIYDASNLETGVPSRF | |
| | AQNLQGRVTMTRDTSTSTVY | | SGSGSGTDFTLTISSLQPEDFA | |
| | MELSSLRSEDTAVYYCAIGDY | | TYYCQQANSFPLTFGQGTKV | |
| | FDYWGQGTLVTVSS | | EIKR | |
| 73 | EVQLLESGGGLVQPGGSLRLS | 667 | DIQMTQSPSSLSASVGDRVTI | 689 |
| | CAASGFTVSSNYMSWVRQAP | | TCRASQSISTYLNWYQQKPG | |
| | GKGLEWVAVIYSDGKTYYAD | | KAPKLLIYDASNLETGVPSRF | |
| | SVKGRFTISRDNSKNTLYLQM | | SGSGSGTDFTLTISSLQPEDFA | |
| | NSLRAEDTAVYYCAREDSSGS | | TYYCQQAHSFPPTFGQGTRLE | |
| | HFDYWGQGTLVTVSS | | IKR | |
| 74 | QVQLVQSGAEVKKPGSSVKV | 668 | DIQMTQSPSSLSASVGDRVTI | 690 |
| | SCKASGYTFTKYEINWVRQAP | | TCRASQGISNNLNWYQQKPG | |
| | GQGLEWMGGIIPIFGTANYAQ | | KAPKLLIYDASYLETGVPSRF | |
| | KFQGRVTITADESTSTAYMEL | | SGSGSGTDFTLTISSLQPEDFA | |
| | SSLRSEDTAVYYCARGSGWY | | TYYCQQSYSAPLTFGQGTKV | |
| | TPLFDYWGQGTLVTVSS | | EIKR | |
| 75 | QVQLVQSGAEVKKPGASVKV | 669 | EIVMTQSPATLSVSPGERATL | 691 |
| | SCKASGYTFTDYYIHWVRQAP | | SCRASQSVSSYLAWYQQKPG | |
| | GQGLEWMGLIDPSGGSTSIAQ | | QAPRLLIYDASARATGIPARF | |
| | KFQGRVTMTRDTSTSTVYME | | SGSGSGTEFTLTISSLQSEDFA | |
| | LSSLRSEDTAVYYCARDYDIL | | VYYCQQYRSSVTFGQGTRLEI | |
| | TGSGFDPWGQGTLVTVSS | | KR | |
| 76 | QVQLVQSGAEVKKPGASVKV | 670 | DIQMTQSPSSLSASVGDRVTI | 692 |
| | SCKASGYTFTTYYMHWVRQA | | TCQASQDINNYLNWYQQKPG | |
| | PGQGLEWMGIINVSAGTTSYA | | KAPKLLIYDASNLETGVPSRF | |
| | QKFQGRVTMTRDTSTSTVYM | | SGSGSGTDFTLTISSLQPEDFA | |
| | ELSSLRSEDTAVYYCAKEPYP | | TYYCQQANSFPLTFGGGTKV | |
| | HQSGWFFDYWGQGTLVTVSS | | EIKR | |
| 77 | QVQLVQSGAEVKKPGASVKV | 671 | EIVMTQSPATLSVSPGERATL | 693 |
| | SCKASGYTFTGHYMHWVRQA | | SCSASQSVGSSYFAWYQQKP | |
| | PGQGLEWMGWISTDNGNANY | | GQAPRLLIYDVSTRATGIPAR | |
| | AQKFQGRVTMTRDTSTSTVY | | FSGSGSGTEFTLTISSLQSEDF | |
| | MELSSLRSEDTAVYYCARDTA | | AVYYCQQYYSTPLTFGPGTK | |
| | DYYFDYWGQGTLVTVSS | | VDIKR | |
| 78 | QVQLVQSGAEVKKPGSSVKV | 672 | DIQMTQSPSSLSASVGDRVTI | 694 |
| | SCKASGGTFSRYPFSWVRQAP | | TCQASQDISNYLNWYQQKPG | |
| | GQGLEWMGWMNPNNGDTGY | | KAPKLLIYDASNLETGVPSRF | |
| | AQKFQGRVTITADESTSTAYM | | SGSGSGTDFTLTISSLQPEDFA | |
| | ELSSLRSEDTAVYYCARGDYP | | TYYCQQSYSIPYTFGQGTKLE | |
| | YMDVWGKGTTVTVSS | | IKR | |
| 79 | QVQLVQSGAEVKKPGASVKV | 673 | DIQMTQSPSSLSASVGDRVTI | 695 |
| | SCKASGYTFTSDYMHWVRQA | | TCRASQGIRNDLGWYQQKPG | |
| | PGQGLEWMGWMNPNSGGTN | | KAPKLLIYAASSLQPGVPSRF | |
| | YAQKFQGRVTMTRDTSTSTV | | SGSGSGTDFTLTISSLQPEDFA | |
| | YMELSSLRSEDTAVYYCARD | | TYYCLQTNSFPWTFGQGTKL | |
| | YITGPSDWGQGTLVTVSS | | EIKR | |
| 80 | QVQLVQSGAEVKKPGASVKV | 674 | DIQMTQSPSSLSASVGDRVTI | 696 |
| | SCKASGFTFTSYYMHWVRQA | | TCRASQSISSWLAWYQQKPG | |
| | PGQGLEWMGWMNPNSGNTG | | KAPKLLIYDTSSLQSGVPSRFS | |
| | YAQRFQGRVTMTRDTSTSTV | | GSGSGTDFTLTISSLQPEDFAT | |
| | YMELSSLRSEDTAVYYCARG | | YYCQQGYSTPLTFGQGTKVEI | |
| | HSRTDYGMDVWGQGTTVTVS | | KR | |
| | S | | | |
| 81 | EVQLLESGGGLVQPGGSLRLS | 675 | DIQMTQSPSSLSASVGDRVTI | 697 |
| | CAASGFTFSDHYMSWVRQAP | | TCQASQDISNYLNWYQQKPG | |
| | GKGLEWVSIIYPDGKTYYADS | | KAPKLLIYGASTLQSGVPSRF | |
| | VKGRFTISRDNSKNTLYLQMN | | SGSGSGTDFTLTISSLQPEDFA | |
| | SLRAEDTAVYYCAREGSYGD | | TYYCQQSYSTPWTFGQGTKL | |
| | YDGMDVWGQGTTVTVSS | | EIKR | |
| 82 | QVQLVQSGAEVKKPGSSVKV | 676 | EIVMTQSPATLSVSPGERATL | 698 |
| | SCKASGGTFSNYDISWVRQAP | | SCRASQSVSSYLAWYQQKPG | |
| | GQGLEWMGGIIPIFGTANYAQ | | QAPRLLIYGASTRATGIPARFS | |
| | KFQGRVTITADESTSTAYMEL | | GSGSGTEFTLTISSLQSEDFAV | |
| | SSLRSEDTAVYYCAREAEEGG | | YYCQQYAFSPITFGQGTKLEI | |
| | WFDPWGQGTLVTVSS | | KR | |
| 83 | QVQLVQSGAEVKKPGASVKV | 677 | DIQMTQSPSSLSASVGDRVTI | 699 |
| | SCKASGYTFTDYYMHWVRQA | | TCRVSQGISSYLNWYQQKPG | |
| | PGQGLEWMGWMNPNSGYTA | | KAPKLLIYDASNLETGVPSRF | |
| | YAQKFQGRVTMTRDTSTSTV | | SGSGSGTDFTLTISSLQPEDFA | |
| | YMELSSLRSEDTAVYYCAKD | | TYYCQQSYSTPLTFGGGTKV | |
| | TPGSGWSSGMDVWGQGTTVT | | EIKR | |
| | VSS | | | |
| 84 | QVQLVQSGAEVKKPGASVKV | 678 | DIQMTQSPSSLSASVGDRVTI | 700 |
| | SCKASGGTFSNYAISWVRQAP | | TCRASQSISSWLAWYQQKPG | |
| | GQGLEWMGWINPNSGGTNYA | | KAPKLLIYDASNLETGVPSRF | |
| | QKFQGRVTMTRDTSTSTVYM | | SGSGSGTDFTLTISSLQPEDFA | |
| | ELSSLRSEDTAVYYCARVGYY | | TYYCLQTHSFPLTFGPGTKVD | |
| | DSSGGGMDVWGQGTTVTVSS | | IKR | |
| 85 | QVQLVQSGAEVKKPGASVKV | 679 | DIQMTQSPSSLSASVGDRVTI | 701 |
| | SCKASGYTFTGYYMHWVRQA | | TCRASQSVSNWLAWYQQKP | |
| | PGQGLEWMGIINPIGGLTTYA | | GKAPKLLIYDASNLQTGVPSR | |
| | QKFQGRVTMTRDTSTSTVYM | | FSGSGSGTDFTLTISSLQPEDF | |
| | ELSSLRSEDTAVYYCASGAYG | | ATYYCQQANSFPLTFGGGTK | |
| | DYVDWYFDLWGRGTLVTVSS | | LEIKR | |
| 86 | QVQLVQSGAEVKKPGASVKV | 680 | DIVMTQSPLSLPVTPGEPASIS | 702 |
| | SCKASGYTFTTYGISWVRQAP | | CRSSRSLLHSNGYNYLDWYL | |
| | GQGLEWMGWINPNSGDTNYA | | QKPGQSPQLLIYLGSYRASGV | |
| | QKFQGRVTMTRDTSTSTVYM | | PDRFSGSGSGTDFTLKISRVE | |
| | ELSSLRSEDTAVYYCARLTTA | | AEDVGVYYCMQGTHWPPTF | |
| | TDSFDLWGRGTLVTVSS | | GQGTKLEIKR | |
| 87 | QVQLVQSGAEVKKPGASVKV | 681 | DIQMTQSPSSLSASVGDRVTI | 703 |
| | SCKASGYSFTNYYIHWVRQAP | | TCZASQSISSYLNWYQQKPG | |
| | GQGLEWMGWMNPYTGQTGY | | KAPKLLIYDASNLETGVPSRF | |
| | AQKFQGRVTMTRDTSTSTVY | | SGSGSGTDFTLTISSLQPEDFA | |
| | MELSSLRSEDTAVYYCTTDEE | | TYYCQQANTFPITFGQGTRLE | |
| | TMDFHLWGRGTLVTVSS | | IKR | |
| 88 | QVQLVQSGAEVKKPGASVKV | 682 | DIVMTQSPLSLPVTPGEPASIS | 704 |
| | SCKASGYTFTGYHIHWVRQAP | | CRSSRSLLHSNGYNYLDWYL | |
| | GQGLEWMGRINPNSGGTDYA | | QKPGQSPQLLIYLGSDRASGV | |
| | QKFQGRVTMTRDTSTSTVYM | | PDRFSGSGSGTDFTLKISRVE | |
| | ELSSLRSEDTAVYYCARETYS | | AEDVGVYYCMQGTHWPPTF | |
| | GSYEESFDYWGQGTLVTVSS | | GQGTKVEIKR | |

### Example 4: Flow Cytometry (FACS)

For CD33. Jurkat cells were engineered to stably express either the huCD33-R69 or huCD33-G69 variant at >200,000 receptors per cell. Parental, huCD33-R69, and huCD33-G69 Jurkat cell lines were stained with differing levels of CellTrace Violet Cell Proliferation Kit (ThermoFisher, cat. #C34557) to barcode each cell line. Barcoded Jurkat cell lines were fixed with paraformaldehyde and incubated with myc-labeled scFv periplasmic extracts and a secondary anti-myc PE-conjugated monoclonal antibody. Appropriate positive and negative controls were used. Stained cells were analyzed by flow cytometry (CytoFLEX, Beckman Coulter, Inc.) and binding was assessed by change in PE mean fluorescence intensity (MFI) of the barcoded cell populations.

For FLT3, Ramos cells were engineered to stably express either the huFLT3-T227 or huFLT3-M227 variant at >200,000 receptors per cell. Parental, huFLT3-T227, and huFLT3-M227 Ramos cell lines were stained with differing levels of CellTrace Violet Cell Proliferation Kit (ThermoFisher, cat. #C34557) to barcode each cell line. Barcoded Ramos cell lines were fixed with paraformaldehyde and incubated with myc-labeled scFv periplasmic extracts and a secondary anti-myc PE-conjugated monoclonal antibody. Appropriate positive and negative controls were used. Stained cells were analyzed by flow cytometry (CytoFLEX, Beckman Coulter, Inc.) and binding was assessed by change in PE mean fluorescence intensity (MFI) of the barcoded cell populations.

Results from this assay for CD33 are shown in Table 8a, reporting fold change over parental as (−), indicating <2 fold; (+), indicating 2-10 fold; (++), indicating 10-30 fold; and (+++), indicating >30 fold. Data are also visualized in FIGS. 2 and 3.

TABLE 8a
Polypeptide Selectivity - CD33

| | CD33 G69 | CD33 R69 |
|---|---|---|
| | Geometric Mean | Geometric Mean |
| | Fold Change over | Fold Change over |
| Polypeptide No. | Jurkat Parental | Jurkat Parental |
| 1 | − | +++ |
| 2 | − | ++ |
| 3 | − | +++ |
| 4 | − | +++ |
| 5 | − | +++ |
| 6 | − | +++ |
| 7 | − | ++ |
| 8 | − | ++ |
| 9 | − | + |
| 10 | − | + |
| 11 | − | + |
| 12 | − | + |
| 13 | − | ++ |
| 14 | − | +++ |
| 15 | − | + |
| 16 | − | + |
| 17 | − | + |
| 18 | − | + |
| 19 | − | +++ |
| 20 | − | ++ |
| 21 | − | + |
| 22 | − | + |
| 23 | − | + |
| 24 | − | +++ |
| 25 | − | +++ |
| 26 | + | − |
| 27 | + | − |
| 28 | ++ | − |
| 29 | + | − |
| 30 | ++ | − |
| 31 | ++ | − |
| 32 | ++ | − |
| 33 | +++ | − |
| 34 | + | − |
| 35 | + | − |
| 36 | + | − |
| 37 | + | − |
| 38 | +++ | − |
| 39 | ++ | − |
| 40 | ++ | − |
| 41 | + | − |
| 42 | ++ | − |

The foregoing methods may be adapted to demonstrate the binding and polymorphic selectivity of other scFvs against antigens such as cancer antigens. For example, the methods are expected to demonstrate anti-FLT3 scFvs that selectively bind either the T227 or T227M polymorphism.

### Example 5: Bio-Layer Interferometry (BLI)

Discovered scFvs were analyzed for binding to huCD33-R69-His or huCD33-G69-Fc recombinant proteins (for anti-CD33 scFvs; for CLL-1 scFvs, huCLL1-K244-Avi-Tev-His or huCLL1-Q244-Avi-Tev-His were used; for FLT3, huFLT3-T227-His or huFLT3-M227-Fc were used) using BLI on a ForteBio Octet HTX instrument. Streptavidin-coated biosensors were loaded with biotinylated anti-V5 tag monoclonal antibody for 5 min and were then quenched and blocked with 20 μM amine-PEG2-Biotin for 5 min. scFvs were captured on biosensors from scFv clone periplasmic extracts. huCD33 (or huCLL-1, or huFLT3) proteins were then associated with the captured scFvs for 2 minutes, followed by dissociation with buffer (1×HBST [10 mM HEPES pH 7.4, 150 mM NaCl, 0.05% Tween-20], 1 g/L BSA) for 5 minutes. Data was buffer referenced subtracted against a negative control scFv and report points were collected at a time point (115-sec or 119-sec) just before the end of the association step to assess yes/no binding. Data was fitted with 1:1 Langmuir equation and off-rate values were reported.

Results from this assay for CD33 are shown in Table 9a, reporting binding, no binding, or ambiguous.

TABLE 9a
Polypeptide Selectivity - CD33

| | CD33 R69 | CD33 G69 |
|---|---|---|
| Polypeptide No. | BLI/Octet Binding | BLI/Octet Binding |
| 1 | Yes | No |
| 2 | Yes | No |
| 3 | Yes | No |
| 4 | Yes | No |
| 5 | Yes | No |
| 6 | Yes | No |
| 7 | Yes | No |
| 8 | Yes | No |
| 9 | Yes | No |
| 10 | Yes | No |
| 11 | Yes | No |
| 12 | Yes | No |
| 13 | Yes | No |
| 14 | Yes | No |
| 15 | Yes | No |
| 16 | Yes | No |
| 17 | Yes | No |
| 18 | Yes | No |
| 19 | Yes | No |
| 20 | Yes | No |
| 21 | Yes | No |
| 22 | Yes | No |
| 23 | Yes | No |
| 24 | Yes | No |
| 25 | Yes | No |
| 26 | No | Yes |
| 27 | No | Yes |
| 28 | No | Yes |
| 29 | No | Yes |
| 30 | No | Yes |
| 31 | No | Yes |
| 32 | No | Yes |
| 33 | No | Yes |
| 34 | No | Yes |
| 35 | No | Yes |
| 36 | No | Yes |
| 37 | No | Yes |
| 38 | No | Yes |
| 39 | No | Yes |
| 40 | No | Yes |
| 41 | No | Yes |
| 42 | No | Yes |

Analogous methods were used to assess selectivity of binding of polypeptides to CLL-1 K244 or CLL-1 Q244. Results from this assay are shown in Table 9b, reporting binding, no binding, or ambiguous.

TABLE 9b
Polypeptide Selectivity - CLL-1

| | CLL-1 K244 | CLL-1 Q244 |
|---|---|---|
| Polypeptide No. | BLI/Octet Binding | BLI/Octet Binding |
| 43 | Yes | No |
| 44 | Yes | No |
| 45 | Yes | No |
| 46 | Yes | No |
| 47 | Yes | No |
| 48 | Yes | No |
| 49 | Yes | No |
| 50 | Yes | No |
| 51 | Yes | No |
| 52 | Yes | No |
| 53 | Yes | No |
| 54 | Yes | No |
| 55 | Yes | No |
| 56 | Yes | No |
| 57 | Yes | No |
| 58 | Yes | No |
| 59 | Yes | No |
| 60 | Yes | No |
| 61 | Yes | No |
| 62 | Yes | No |
| 63 | Yes | No |
| 64 | Yes | No |
| 65 | Yes | No |
| 66 | Yes | No |
| 67 | No | Yes |
| 68 | No | Yes |
| 69 | No | Yes |
| 70 | No | Yes |
| 71 | No | Yes |
| 72 | No | Yes |
| 73 | No | Yes |
| 74 | No | Yes |
| 75 | No | Yes |
| 76 | No | Yes |
| 77 | No | Yes |
| 78 | No | Yes |
| 79 | No | Yes |
| 80 | No | Yes |
| 81 | No | Yes |
| 82 | No | Yes |
| 83 | No | Yes |
| 84 | No | Yes |
| 85 | No | Yes |
| 86 | No | Yes |
| 87 | No | Yes |
| 88 | No | Yes |

### Example 6: Chimeric Antigen Receptors Comprising ScFvs

Below in Tables 10 and Table 11 are provided examples of chimeric antigen receptors comprising scFvs as disclosed herein that may be constructed and expressed in immune effector cells according to methods known in the art and disclosed herein (CAR Examples 1-60). Tables 10 and 11 are intended to provide examples of how CARs comprising the V_{H} and V_{L} chains of the scFvs disclosed herein may be constructed. Further CARs may be constructed from other scFv V_{H} and V_{L} chains disclosed herein.

The CARs in Table 10 below are of the form:
   |-[(signal)(scFv V_{H})(linker)(scFv V_{L})(hinge)(TMD)(costim)(effector)]-|+ (tag)
   or
   |-[(signal)(scFv V_{L})(linker)(scFv V_{H})(hinge)(TMD)(costim)(effector)]-|+ (tag),
   wherein:
the CD8a signal sequence MALPVTALLLPLALLLHAARP has SEQ ID NO: 1521, or alternatively, one of SEQ ID NO.s 1522-1525 may be used;
the (GGGGS)₄ linker has SEQ ID NO: 1536, or alternatively, one of SEQ ID NO.s 1532-1535 may be used;
the CD8 hinge sequence TTTPAPRPPTPAPTIASQPLSLRPEACRPAAGGAVHTR GLDFACD has SEQ ID NO: 1526;
the CD28 transmembrane domain sequence FWVLVVVGGVLACYSLLVTVAFIIFWV has SEQ ID NO: 1527;
the CD28 costim domain sequence RSKRSRLLHSDYMNMTPRRPGPTRKHYQPYAP PRDFAAYRS has SEQ ID NO: 1530;
the 4-1BB costim domain sequence KRGRKKLLYIFKQPFMRPVQTTQEEDGCSCRF PEEEEGGCEL has SEQ ID NO: 1529;
the CD3z effector domain sequence RVKFSRSADAPAYKQGQNQLYNELNLGRREE YDVLDKRRGRDPEMGGKPRRKNPQEGLYNELQKDKMAEAYSEIGMKGERRRG KGHDGLYQGLSTATKDTYDALHMQALPPR has SEQ ID NO: 1531;
P2A sequence GSGATNFSLLKQAGDVEENPGP has SEQ ID NO: 1532;
the CD34 tag sequence MPRGWTALCLLSLLPSGFMSLDNNGTATPELPTQGTFSNV STNVSYQETTTPSTLGSTSLHPVSQHGNEATTNITETTVKFTSTSVITSVYGNTNSS VQSQTSVISTVFTTPANVSTPETTLKPSLSPGNVSDLSTTSTSLATSPTKPYTSSSPI LSDIKAEIKCSGIREVKLTQGICLEQNKTSSCAEFKKDRGEGLARVLCGEEQADA DAGAQVCSLLLAQSEVRPQCLLLVLANRTEISSKLQLMKKHQSDLKKLGILDFTE QDVASHQSYSQKTLIALVTSGALLAVLGITGYFLMNRRSWSPI has SEQ ID NO: 1537, or alternatively, a variant or truncated CD34 sequence may be used;
the V_{H} and V_{L} domains of Ex.s 1-3 and 31-33, are from Polypeptide 1, e.g., SEQ ID NO: 151 and SEQ ID NO: 176, respectively;
the V_{H} and V_{L} domains of Ex.s 4-6 and 34-36, are from Polypeptide 5, e.g., SEQ ID NO: 155 and SEQ ID NO: 180, respectively;
the V_{H} and V_{L} domains of Ex.s 7-9 and 37-39, are from Polypeptide 6, e.g., SEQ ID NO: 156 and SEQ ID NO: 181, respectively;
the V_{H} and V_{L} domains of Ex.s 10-12 and 40-42, are from Polypeptide 7, e.g., SEQ ID NO: 157 and SEQ ID NO: 182, respectively;
the V_{H} and V_{L} domains of Ex.s 13-15 and 43-45, are from Polypeptide 25, e.g., SEQ ID NO: 175 and SEQ ID NO: 200, respectively;
the V_{H} and V_{L} domains of Ex.s 16-18 and 46-48, are from Polypeptide 30, e.g., SEQ ID NO: 307 and SEQ ID NO: 324, respectively;
the V_{H} and V_{L} domains of Ex.s 19-21 and 49-51, are from Polypeptide 31, e.g., SEQ ID NO: 308 and SEQ ID NO: 325, respectively;
the V_{H} and V_{L} domains of Ex.s 22-24 and 52-54, are from Polypeptide 32, e.g., SEQ ID NO: 309 and SEQ ID NO: 326, respectively;
the V_{H} and V_{L} domains of Ex.s 25-27 and 55-57, are from Polypeptide 33, e.g., SEQ ID NO: 310 and SEQ ID NO: 337, respectively; and
the V_{H} and V_{L} domains of Ex.s 28-30 and 58-60, are from Polypeptide 38, e.g., SEQ ID NO: 315 and SEQ ID NO: 332, respectively.

TABLE 10
CAR Constructs

| | CD8a | VH or | (GGGGS)⁴ | VH or | CD8 | CD28 | CD28 | 4-1BB | CD3z |
|---|---|---|---|---|---|---|---|---|---|
| | Signal | VL | Linker | VL | Hinge | TMD | CoStim | CoStim | Effector |
| CAR | SEQ | SEQ ID | SEQ ID | SEQ ID | SEQ | SEQ | SEQ | SEQ | SEQ ID |
| Ex. | ID NO | NO | NO | NO | ID NO | ID NO | ID NO | ID NO | NO |
| 1 | 1521 | 151 | 1536 | 176 | 1526 | 1527 | 1530 | — | 1531 |
| 2 | 1521 | 151 | 1536 | 176 | 1526 | 1527 | — | 1529 | 1531 |
| 3 | 1521 | 151 | 1536 | 176 | 1526 | 1527 | 1530 | 1529 | 1531 |
| 4 | 1521 | 155 | 1536 | 180 | 1526 | 1527 | 1530 | — | 1531 |
| 5 | 1521 | 155 | 1536 | 180 | 1526 | 1527 | — | 1529 | 1531 |
| 6 | 1521 | 155 | 1536 | 180 | 1526 | 1527 | 1530 | 1529 | 1531 |
| 7 | 1521 | 156 | 1536 | 181 | 1526 | 1527 | 1530 | — | 1531 |
| 8 | 1521 | 156 | 1536 | 181 | 1526 | 1527 | — | 1529 | 1531 |
| 9 | 1521 | 156 | 1536 | 181 | 1526 | 1527 | 1530 | 1529 | 1531 |
| 10 | 1521 | 157 | 1536 | 182 | 1526 | 1527 | 1530 | — | 1531 |
| 11 | 1521 | 157 | 1536 | 182 | 1526 | 1527 | — | 1529 | 1531 |
| 12 | 1521 | 157 | 1536 | 182 | 1526 | 1527 | 1530 | 1529 | 1531 |
| 13 | 1521 | 175 | 1536 | 200 | 1526 | 1527 | 1530 | — | 1531 |
| 14 | 1521 | 175 | 1536 | 200 | 1526 | 1527 | — | 1529 | 1531 |
| 15 | 1521 | 175 | 1536 | 200 | 1526 | 1527 | 1530 | 1529 | 1531 |
| 16 | 1521 | 307 | 1536 | 324 | 1526 | 1527 | 1530 | — | 1531 |
| 17 | 1521 | 307 | 1536 | 324 | 1526 | 1527 | — | 1529 | 1531 |
| 18 | 1521 | 307 | 1536 | 324 | 1526 | 1527 | 1530 | 1529 | 1531 |
| 19 | 1521 | 308 | 1536 | 325 | 1526 | 1527 | — | 1529 | 1531 |
| 20 | 1521 | 308 | 1536 | 325 | 1526 | 1527 | 1530 | 1529 | 1531 |
| 21 | 1521 | 308 | 1536 | 325 | 1526 | 1527 | 1530 | — | 1531 |
| 22 | 1521 | 309 | 1536 | 326 | 1526 | 1527 | — | 1529 | 1531 |
| 23 | 1521 | 309 | 1536 | 326 | 1526 | 1527 | 1530 | 1529 | 1531 |
| 24 | 1521 | 309 | 1536 | 326 | 1526 | 1527 | 1530 | — | 1531 |
| 25 | 1521 | 310 | 1536 | 327 | 1526 | 1527 | — | 1529 | 1531 |
| 26 | 1521 | 310 | 1536 | 327 | 1526 | 1527 | 1530 | 1529 | 1531 |
| 27 | 1521 | 310 | 1536 | 327 | 1526 | 1527 | 1530 | — | 1531 |
| 28 | 1521 | 315 | 1536 | 332 | 1526 | 1527 | — | 1529 | 1531 |
| 29 | 1521 | 315 | 1536 | 332 | 1526 | 1527 | 1530 | 1529 | 1531 |
| 30 | 1521 | 315 | 1536 | 332 | 1526 | 1527 | 1530 | — | 1531 |
| 31 | 1521 | 176 | 1536 | 151 | 1526 | 1527 | 1530 | | 1531 |
| 32 | 1521 | 176 | 1536 | 151 | 1526 | 1527 | | 1529 | 1531 |
| 33 | 1521 | 176 | 1536 | 151 | 1526 | 1527 | 1530 | 1529 | 1531 |
| 34 | 1521 | 180 | 1536 | 155 | 1526 | 1527 | 1530 | | 1531 |
| 35 | 1521 | 180 | 1536 | 155 | 1526 | 1527 | | 1529 | 1531 |
| 36 | 1521 | 180 | 1536 | 155 | 1526 | 1527 | 1530 | 1529 | 1531 |
| 37 | 1521 | 181 | 1536 | 156 | 1526 | 1527 | 1530 | | 1531 |
| 38 | 1521 | 181 | 1536 | 156 | 1526 | 1527 | | 1529 | 1531 |
| 39 | 1521 | 181 | 1536 | 156 | 1526 | 1527 | 1530 | 1529 | 1531 |
| 40 | 1521 | 182 | 1536 | 157 | 1526 | 1527 | 1530 | | 1531 |
| 41 | 1521 | 182 | 1536 | 157 | 1526 | 1527 | | 1529 | 1531 |
| 42 | 1521 | 182 | 1536 | 157 | 1526 | 1527 | 1530 | 1529 | 1531 |
| 43 | 1521 | 200 | 1536 | 175 | 1526 | 1527 | 1530 | | 1531 |
| 44 | 1521 | 200 | 1536 | 175 | 1526 | 1527 | | 1529 | 1531 |
| 45 | 1521 | 200 | 1536 | 175 | 1526 | 1527 | 1530 | 1529 | 1531 |
| 46 | 1521 | 324 | 1536 | 307 | 1526 | 1527 | 1530 | | 1531 |
| 47 | 1521 | 324 | 1536 | 307 | 1526 | 1527 | | 1529 | 1531 |
| 48 | 1521 | 324 | 1536 | 307 | 1526 | 1527 | 1530 | 1529 | 1531 |
| 49 | 1521 | 325 | 1536 | 308 | 1526 | 1527 | | 1529 | 1531 |
| 50 | 1521 | 325 | 1536 | 308 | 1526 | 1527 | 1530 | 1529 | 1531 |
| 51 | 1521 | 325 | 1536 | 308 | 1526 | 1527 | 1530 | | 1531 |
| 52 | 1521 | 326 | 1536 | 309 | 1526 | 1527 | | 1529 | 1531 |
| 53 | 1521 | 326 | 1536 | 309 | 1526 | 1527 | 1530 | 1529 | 1531 |
| 54 | 1521 | 326 | 1536 | 309 | 1526 | 1527 | 1530 | | 1531 |
| 55 | 1521 | 327 | 1536 | 310 | 1526 | 1527 | | 1529 | 1531 |
| 56 | 1521 | 327 | 1536 | 310 | 1526 | 1527 | 1530 | 1529 | 1531 |
| 57 | 1521 | 327 | 1536 | 310 | 1526 | 1527 | 1530 | | 1531 |
| 58 | 1521 | 332 | 1536 | 315 | 1526 | 1527 | | 1529 | 1531 |
| 59 | 1521 | 332 | 1536 | 315 | 1526 | 1527 | 1530 | 1529 | 1531 |
| 60 | 1521 | 332 | 1536 | 315 | 1526 | 1527 | 1530 | | 1531 |

Accordingly, provided herein are chimeric antigen receptors comprising the sequences disclosed in the following illustrative examples.

TABLE 11
CAR Sequences

| CAR Ex. | SEQ ID | |
|---|---|---|
| No. | NO. | CAR Sequence |
| 1 | 1539 | MALPVTALLLPLALLLHAARPDIQMTQSPSSLSASVGDRVTITCRASQTINDW |
| | | LAWYQQKPGKAPKLLIYSASTLHSGVPSRFSGSGSGTDFTLTISSLQPEDFATY |
| | | YCQQAYSTPWTFGQGTKVEIKRTTTPAPRPPTPAPTIASQPLSLRPEACRPAA |
| | | GGAVHTRGLDFACDQVQLVQSGAEVKKPGASVKVSCKASGYSFTGYYIHW |
| | | VRQAPGQGLEWMGWINPNSGGTNYAQKFQGRVTMTRDTSTSTVYMELSSL |
| | | RSEDTAVYYCARDQWDGYNSGYFDYWGQGTLVTVSSGGGGSGGGGSGGG |
| | | GSGGGGSFWVLVVVGGVLACYSLLVTVAFIIFWVRSKRSRLLHSDYMNMTP |
| | | RRPGPTRKHYQPYAPPRDFAAYRSRVKFSRSADAPAYKQGQNQLYNELNLG |
| | | RREEYDVLDKRRGRDPEMGGKPRRKNPQEGLYNELQKDKMAEAYSEIGMK |
| | | GERRRGKGHDGLYQGLSTATKDTYDALHMQALPPR |
| 2 | 1540 | MALPVTALLLPLALLLHAARPDIQMTQSPSSLSASVGDRVTITCRASQTINDW |
| | | LAWYQQKPGKAPKLLIYSASTLHSGVPSRFSGSGSGTDFTLTISSLQPEDFATY |
| | | YCQQAYSTPWTFGQGTKVEIKRTTTPAPRPPTPAPTIASQPLSLRPEACRPAA |
| | | GGAVHTRGLDFACDQVQLVQSGAEVKKPGASVKVSCKASGYSFTGYYIHW |
| | | VRQAPGQGLEWMGWINPNSGGTNYAQKFQGRVTMTRDTSTSTVYMELSSL |
| | | RSEDTAVYYCARDQWDGYNSGYFDYWGQGTLVTVSSGGGGSGGGGSGGG |
| | | GSGGGGSFWVLVVVGGVLACYSLLVTVAFIIFWVKRGRKKLLYIFKQPFMRP |
| | | VQTTQEEDGCSCRFPEEEEGGCELRVKFSRSADAPAYKQGQNQLYNELNLG |
| | | RREEYDVLDKRRGRDPEMGGKPRRKNPQEGLYNELQKDKMAEAYSEIGMK |
| | | GERRRGKGHDGLYQGLSTATKDTYDALHMQALPPR |
| 3 | 1541 | MALPVTALLLPLALLLHAARPDIQMTQSPSSLSASVGDRVTITCRASQTINDW |
| | | LAWYQQKPGKAPKLLIYSASTLHSGVPSRFSGSGSGTDFTLTISSLQPEDFATY |
| | | YCQQAYSTPWTFGQGTKVEIKRTTTPAPRPPTPAPTIASQPLSLRPEACRPAA |
| | | GGAVHTRGLDFACDQVQLVQSGAEVKKPGASVKVSCKASGYSFTGYYIHW |
| | | VRQAPGQGLEWMGWINPNSGGTNYAQKFQGRVTMTRDTSTSTVYMELSSL |
| | | RSEDTAVYYCARDQWDGYNSGYFDYWGQGTLVTVSSGGGGSGGGGSGGG |
| | | GSGGGGSFWVLVVVGGVLACYSLLVTVAFIIFWVRSKRSRLLHSDYMNMTP |
| | | RRPGPTRKHYQPYAPPRDFAAYRSKRGRKKLLYIFKQPFMRPVQTTQEEDGC |
| | | SCRFPEEEEGGCELRVKFSRSADAPAYKQGQNQLYNELNLGRREEYDVLDK |
| | | RRGRDPEMGGKPRRKNPQEGLYNELQKDKMAEAYSEIGMKGERRRGKGHD |
| | | GLYQGLSTATKDTYDALHMQALPPR |
| 4 | 1542 | MALPVTALLLPLALLLHAARPDIVMTQSPLSLPVTPGEPASISCRSSQSLLHSN |
| | | GYNYLDWYLQKPGQSPQLLIYLGSDRASGVPDRFSGSGSGTDFTLKISRVEA |
| | | EDVGVYYCMQGLQTPITFGQGTRLEIKRTTTPAPRPPTPAPTIASQPLSLRPEA |
| | | CRPAAGGAVHTRGLDFACDQVQLVQSGAEVKKPGASVKVSCKASGNTFTSY |
| | | GISWVRQAPGQGLEWMGWINPNSGGTKYAQKFQGRVTMTRDTSTSTVYME |
| | | LSSLRSEDTAVYYCARESWFGELYYGMDVWGKGTTVTVSSGGGGSGGGGS |
| | | GGGGSGGGGSFWVLVVVGGVLACYSLLVTVAFIIFWVRSKRSRLLHSDYMN |
| | | MTPRRPGPTRKHYQPYAPPRDFAAYRSRVKFSRSADAPAYKQGQNQLYNEL |
| | | NLGRREEYDVLDKRRGRDPEMGGKPRRKNPQEGLYNELQKDKMAEAYSEI |
| | | GMKGERRRGKGHDGLYQGLSTATKDTYDALHMQALPPR |
| 5 | 1543 | MALPVTALLLPLALLLHAARPDIVMTQSPLSLPVTPGEPASISCRSSQSLLHSN |
| | | GYNYLDWYLQKPGQSPQLLIYLGSDRASGVPDRFSGSGSGTDFTLKISRVEA |
| | | EDVGVYYCMQGLQTPITFGQGTRLEIKRTTTPAPRPPTPAPTIASQPLSLRPEA |
| | | CRPAAGGAVHTRGLDFACDQVQLVQSGAEVKKPGASVKVSCKASGNTFTSY |
| | | GISWVRQAPGQGLEWMGWINPNSGGTKYAQKFQGRVTMTRDTSTSTVYME |
| | | LSSLRSEDTAVYYCARESWFGELYYGMDVWGKGTTVTVSSGGGGSGGGGS |
| | | GGGGSGGGGSFWVLVVVGGVLACYSLLVTVAFIIFWVKRGRKKLLYIFKQPF |
| | | MRPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYSEI |
| | | GMKGERRRGKGHDGLYQGLSTATKDTYDALHMQALPPR |
| 6 | 1544 | MALPVTALLLPLALLLHAARPDIVMTQSPLSLPVTPGEPASISCRSSQSLLHSN |
| | | GYNYLDWYLQKPGQSPQLLIYLGSDRASGVPDRFSGSGSGTDFTLKISRVEA |
| | | EDVGVYYCMQGLQTPITFGQGTRLEIKRTTTPAPRPPTPAPTIASQPLSLRPEA |
| | | CRPAAGGAVHTRGLDFACDQVQLVQSGAEVKKPGASVKVSCKASGNTFTSY |
| | | GISWVRQAPGQGLEWMGWINPNSGGTKYAQKFQGRVTMTRDTSTSTVYME |
| | | LSSLRSEDTAVYYCARESWFGELYYGMDVWGKGTTVTVSSGGGGSGGGGS |
| | | GGGGSGGGGSFWVLVVVGGVLACYSLLVTVAFIIFWVRSKRSRLLHSDYMN |
| | | MTPRRPGPTRKHYQPYAPPRDFAAYRSKRGRKKLLYIFKQPFMRPVQTTQEE |
| | | DGCCRFPEEEEGGCELRVKFSRSADAPAYKQGQNQLYNELNLGRREEYDV |
| | | LDKRRGRDPEMGGKPRRKNPQEGLYNELQKDKMAEAYSEIGMKGERRRGK |
| | | GHDGLYQGLSTATKDTYDALHMQALPPR |
| 7 | 1545 | MALPVTALLLPLALLLHAARPDIQMTQSPSSLSASVGDRVTITCRASQSISSYL |
| | | NWYQQKPGKAPKLLIYEASTLETGVPSRFSGSGSGTDFTLTISSLQPEDFATY |
| | | YCQQANSFPFTFGPGTKVDIKRTTTPAPRPPTPAPTIASQPLSLRPEACRPAAG |
| | | GAVHTRGLDFACDQVQLVQSGAEVKKPGASVKVSCKASGYTFTAYYTHWV |
| | | RQAPGQGLEWMGWMNPNSGHTSYAQKFQGRVTMTRDTSTSTVYMELSSLR |
| | | SEDTAVYYCAREAYDSFDYWGQGTLVTVSSGGGGSGGGGSGGGGSGGGGS |
| | | FWVLVVVGGVLACYSLLVTVAFIIFWVRSKRSRLLHSDYMNMTPRRPGPTR |
| | | KHYQPYAPPRDFAAYRSRVKFSRSADAPAYKQGQNQLYNELNLGRREEYDV |
| | | LDKRRGRDPEMGGKPRRKNPQEGLYNELQKDKMAEAYSEIGMKGERRRGK |
| | | GHDGLYQGLSTATKDTYDALHMQALPPR |
| 8 | 1546 | MALPVTALLLPLALLLHAARPDIQMTQSPSSLSASVGDRVTITCRASQSISSYL |
| | | NWYQQKPGKAPKLLIYEASTLETGVPSRFSGSGSGTDFTLTISSLQPEDFATY |
| | | YCQQANSFPFTFGPGTKVDIKRTTTPAPRPPTPAPTIASQPLSLRPEACRPAAG |
| | | GAVHTRGLDFACDQVQLVQSGAEVKKPGASVKVSCKASGYTFTAYYTHWV |
| | | RQAPGQGLEWMGWMNPNSGHTSYAQKFQGRVTMTRDTSTSTVYMELSSLR |
| | | SEDTAVYYCAREAYDSFDYWGQGTLVTVSSGGGGSGGGGSGGGGSGGGGS |
| | | FWVLVVVGGVLACYSLLVTVAFIIFWVKRGRKKLLYIFKQPFMRPVQTTQEE |
| | | DGCSCRFPEEEEGGCELRVKFSRSADAPAYKQGQNQLYNELNLGRREEYDV |
| | | LDKRRGRDPEMGGKPRRKNPQEGLYNELQKDKMAEAYSEIGMKGERRRGK |
| | | GHDGLYQGLSTATKDTYDALHMQALPPR |
| 9 | 1547 | MALPVTALLLPLALLLHAARPDIQMTQSPSSLSASVGDRVTITCRASQSISSYL |
| | | NWYQQKPGKAPKLLIYEASTLETGVPSRFSGSGSGTDFTLTISSLQPEDFATY |
| | | YCQQANSFPFTFGPGTKVDIKRTTTPAPRPPTPAPTIASQPLSLRPEACRPAAG |
| | | GAVHTRGLDFACDQVQLVQSGAEVKKPGASVKVSCKASGYTFTAYYTHWV |
| | | RQAPGQGLEWMGWMNPNSGHTSYAQKFQGRVTMTRDTSTSTVYMELSSLR |
| | | SEDTAVYYCAREAYDSFDYWGQGTLVTVSSGGGGSGGGGSGGGGSGGGGS |
| | | FWVLVVVGGVLACYSLLVTVAFIIFWVRSKRSRLLHSDYMNMTPRRPGPTR |
| | | KHYQPYAPPRDFAAYRSKRGRKKLLYIFKQPFMRPVQTTQEEDGCSCRFPEE |
| | | EEGGCELRVKFSRSADAPAYKQGQNQLYNELNLGRREEYDVLDKRRGRDPE |
| | | MGGKPRRKNPQEGLYNELQKDKMAEAYSEIGMKGERRRGKGHDGLYQGLS |
| | | TATKDTYDALHMQALPPR |
| 10 | 1548 | MALPVTALLLPLALLLHAARPDIQMTQSPSSLSASVGDRVTITCRASRGINNW |
| | | LTWYQQKPGKAPKLLIYGASSLQSGVPSRFSGSGSGTDFTLTISSLQPEDFATY |
| | | YCQQSYRIPYTFGQGTKLEIKRTTTPAPRPPTPAPTIASQPLSLRPEACRPAAG |
| | | GAVHTRGLDFACDQVQLVQSGAEVKKPGASVKVSCKASGYTFTDYYMHW |
| | | VRQAPGQGLEWMGWINPNSGGTNYAQKFQGRVTMTRDTSTSTVYMELSSL |
| | | RSEDTAVYYCARDSRIAVAASSFDYWGQGTLVTVSSGGGGSGGGGSGGGGS |
| | | GGGGSFWVLVVVGGVLACYSLLVTVAFIIFWVRSKRSRLLHSDYMNMTPRR |
| | | PGPTRKHYQPYAPPRDFAAYRSRVKFSRSADAPAYKQGQNQLYNELNLGRR |
| | | EEYDVLDKRRGRDPEMGGKPRRKNPQEGLYNELQKDKMAEAYSEIGMKGE |
| | | RRRGKGHDGLYQGLSTATKDTYDALHMQALPPR |
| 11 | 1549 | MALPVTALLLPLALLLHAARPDIQMTQSPSSLSASVGDRVTITCRASRGINNW |
| | | LTWYQQKPGKAPKLLIYGASSLQSGVPSRFSGSGSGTDFTLTISSLQPEDFATY |
| | | YCQQSYRIPYTFGQGTKLEIKRTTTPAPRPPTPAPTIASQPLSLRPEACRPAAG |
| | | GAVHTRGLDFACDQVQLVQSGAEVKKPGASVKVSCKASGYTFTDYYMHW |
| | | VRQAPGQGLEWMGWINPNSGGTNYAQKFQGRVTMTRDTSTSTVYMELSSL |
| | | RSEDTAVYYCARDSRIAVAASSFDYWGQGTLVTVSSGGGGSGGGGSGGGGS |
| | | GGGGSFWVLVVVGGVLACYSLLVTVAFIIFWVKRGRKKLLYIFKQPFMRPV |
| | | QTTQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMKGE |
| | | RRRGKGHDGLYQGLSTATKDTYDALHMQALPPR |
| 12 | 1550 | MALPVTALLLPLALLLHAARPDIQMTQSPSSLSASVGDRVTITCRASRGINNW |
| | | LTWYQQKPGKAPKLLIYGASSLQSGVPSRFSGSGSGTDFTLTISSLQPEDFATY |
| | | YCQQSYRIPYTFGQGTKLEIKRTTTPAPRPPTPAPTIASQPLSLRPEACRPAAG |
| | | GAVHTRGLDFACDQVQLVQSGAEVKKPGASVKVSCKASGYTFTDYYMHW |
| | | VRQAPGQGLEWMGWINPNSGGTNYAQKFQGRVTMTRDTSTSTVYMELSSL |
| | | RSEDTAVYYCARDSRIAVAASSFDYWGQGTLVTVSSGGGGSGGGGSGGGGS |
| | | GGGGSFWVLVVVGGVLACYSLLVTVAFIIFWVRSKRSRLLHSDYMNMTPRR |
| | | PGPTRKHYQPYAPPRDFAAYRSKRGRKKLLYIFKQPFMRPVQTTQEEDGCSC |
| | | RFPEEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGHDGL |
| | | YQGLSTATKDTYDALHMQALPPR |
| 13 | 1551 | MALPVTALLLPLALLLHAARPDIQMTQSPSSLSASVGDRVTITCRASQSINDW |
| | | LAWYQQKPGKAPKLLIYAASNLQSGVPSRFSGSGSGTDFTLTISSLQPEDFAT |
| | | YYCQQGYSTPPTFGQGTKVEIKRTTTPAPRPPTPAPTIASQPLSLRPEACRPAA |
| | | GGAVHTRGLDFACDQVQLVQSGAEVKKPGASVKVSCKASGYTFTGYYMH |
| | | WVRQAPGQGLEWMGRINPNGGSTTYAQKFQGRVTMTRDTSTSTVYMELSS |
| | | LRSEDTAVYYCARDDFYYYYLDFWGKGTTVTVSSGGGGSGGGGSGGGGSG |
| | | GGGSFWVLVVVGGVLACYSLLVTVAFIIFWVRSKRSRLLHSDYMNMTPRRP |
| | | GPTRKHYQPYAPPRDFAAYRSRVKFSRSADAPAYKQGQNQLYNELNLGRRE |
| | | EYDVLDKRRGRDPEMGGKPRRKNPQEGLYNELQKDKMAEAYSEIGMKGER |
| | | RRGKGHDGLYQGLSTATKDTYDALHMQALPPR |
| 14 | 1552 | MALPVTALLLPLALLLHAARPDIQMTQSPSSLSASVGDRVTITCRASQSINDW |
| | | LAWYQQKPGKAPKLLIYAASNLQSGVPSRFSGSGSGTDFTLTISSLQPEDFAT |
| | | YYCQQGYSTPPTFGQGTKVEIKRTTTPAPRPPTPAPTIASQPLSLRPEACRPAA |
| | | GGAVHTRGLDFACDQVQLVQSGAEVKKPGASVKVSCKASGYTFTGYYMH |
| | | WVRQAPGQGLEWMGRINPNGGSTTYAQKFQGRVTMTRDTSTSTVYMELSS |
| | | LRSEDTAVYYCARDDFYYYYLDFWGKGTTVTVSSGGGGSGGGGSGGGGSG |
| | | GGGSFWVLVVVGGVLACYSLLVTVAFIIFWVKRGRKKLLYIFKQPFMRPVQT |
| | | TQEEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGERR |
| | | RGKGHDGLYQGLSTATKDTYDALHMQALPPR |
| 15 | 1553 | MALPVTALLLPLALLLHAARPDIQMTQSPSSLSASVGDRVTITCRASQSINDW |
| | | LAWYQQKPGKAPKLLIYAASNLQSGVPSRFSGSGSGTDFTLTISSLQPEDFAT |
| | | YYCQQGYSTPPTFGQGTKVEIKRTTTPAPRPPTPAPTIASQPLSLRPEACRPAA |
| | | GGAVHTRGLDFACDQVQLVQSGAEVKKPGASVKVSCKASGYTFTGYYMH |
| | | WVRQAPGQGLEWMGRINPNGGSTTYAQKFQGRVTMTRDTSTSTVYMELSS |
| | | LRSEDTAVYYCARDDFYYYYLDFWGKGTTVTVSSGGGGSGGGGSGGGGSG |
| | | GGGSFWVLVVVGGVLACYSLLVTVAFIIFWVRSKRSRLLHSDYMNMTPRRP |
| | | GPTRKHYQPYAPPRDFAAYRSKRGRKKLLYIFKQPFMRPVQTTQEEDGCSCR |
| | | FPEEEEGGCELRVKFSRSADAPAYKQGQNQLYNELNLGRREEYDVLDKRRG |
| | | RDPEMGGKPRRKNPQEGLYNELQKDKMAEAYSEIGMKGERRRGKGHDGLY |
| | | QGLSTATKDTYDALHMQALPPR |
| 16 | 1554 | MALPVTALLLPLALLLHAARPDIQMTQSPSSLSASVGDRVTITCRASQSISSW |
| | | LAWYQQKPGKAPKLLIYGASSLQSGVPSRFSGSGSGTDFTLTISSLQPEDFAT |
| | | YYCQQTYRTPLTFGPGTKVDIKRTTTPAPRPPTPAPTIASQPLSLRPEACRPAA |
| | | GGAVHTRGLDFACDQVQLVQSGAEVKKPGASVKVSCKASGYTFSNYYMH |
| | | WVRQAPGQGLEWMGWMNPDSGTTGYAQKFQGRVTMTRDTSTSTVYMELS |
| | | SLRSEDTAVYYCVRDGTMVQGIFDYWGQGTLVTVSSGGGGSGGGGSGGGG |
| | | SGGGGSFWVLVVVGGVLACYSLLVTVAFIIFWVRSKRSRLLHSDYMNMTPR |
| | | RPGPTRKHYQPYAPPRDFAAYRSRVKFSRSADAPAYKQGQNQLYNELNLGR |
| | | REEYDVLDKRRGRDPEMGGKPRRKNPQEGLYNELQKDKMAEAYSEIGMKG |
| | | ERRRGKGHDGLYQGLSTATKDTYDALHMQALPPR |
| 17 | 1555 | MALPVTALLLPLALLLHAARPDIQMTQSPSSLSASVGDRVTITCRASQSISSW |
| | | LAWYQQKPGKAPKLLIYGASSLQSGVPSRFSGSGSGTDFTLTISSLQPEDFAT |
| | | YYCQQTYRTPLTFGPGTKVDIKRTTTPAPRPPTPAPTIASQPLSLRPEACRPAA |
| | | GGAVHTRGLDFACDQVQLVQSGAEVKKPGASVKVSCKASGYTFSNYYMH |
| | | WVRQAPGQGLEWMGWMNPDSGTTGYAQKFQGRVTMTRDTSTSTVYMELS |
| | | SLRSEDTAVYYCVRDGTMVQGIFDYWGQGTLVTVSSGGGGSGGGGSGGGG |
| | | SGGGGSFWVLVVVGGVLACYSLLVTVAFIIFWVKRGRKKLLYIFKQPFMRPV |
| | | QTTQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMKGE |
| | | RRRGKGHDGLYQGLSTATKDTYDALHMQALPPR |
| 18 | 1556 | MALPVTALLLPLALLLHAARPDIQMTQSPSSLSASVGDRVTITCRASQSISSW |
| | | LAWYQQKPGKAPKLLIYGASSLQSGVPSRFSGSGSGTDFTLTISSLQPEDFAT |
| | | YYCQQTYRTPLTFGPGTKVDIKRTTTPAPRPPTPAPTIASQPLSLRPEACRPAA |
| | | GGAVHTRGLDFACDQVQLVQSGAEVKKPGASVKVSCKASGYTFSNYYMH |
| | | WVRQAPGQGLEWMGWMNPDSGTTGYAQKFQGRVTMTRDTSTSTVYMELS |
| | | SLRSEDTAVYYCVRDGTMVQGIFDYWGQGTLVTVSSGGGGSGGGGSGGGG |
| | | SGGGGSFWVLVVVGGVLACYSLLVTVAFIIFWVRSKRSRLLHSDYMNMTPR |
| | | RPGPTRKHYQPYAPPRDFAAYRSKRGRKKLLYIFKQPFMRPVQTTQEEDGCS |
| | | CRFPEEEEGGCELRVKFSRSADAPAYKQGQNQLYNELNLGRREEYDVLDKR |
| | | RGRDPEMGGKPRRKNPQEGLYNELQKDKMAEAYSEIGMKGERRRGKGHDG |
| | | LYQGLSTATKDTYDALHMQALPPR |
| 19 | 1557 | MALPVTALLLPLALLLHAARPDIQMTQSPSSLSASVGDRVTITCRASQGIGND |
| | | LGWYQQKPGKAPKLLIYGASSVQSGVPSRFSGSGSGTDFTLTISSLQPEDFAT |
| | | YYCQQSYSTPITFGQGTRLEIKRTTTPAPRPPTPAPTIASQPLSLRPEACRPAAG |
| | | GAVHTRGLDFACDQVQLVQSGAEVKKPGSSVKVSCKASGGTFSTYAITWVR |
| | | QAPGQGLEWMGGIIPIVGRANYAQKFQGRVTITADESTSTAYMELSSLRSED |
| | | TAVYYCARSGGHDLDYWGQGTLVTVSSGGGGSGGGGSGGGGSGGGGSFW |
| | | VLVVVGGVLACYSLLVTVAFIIFWVKRGRKKLLYIFKQPFMRPVQTTQEEDG |
| | | CSCRFPEEEEGGCELRVKFSRSADAPAYKQGQNQLYNELNLGRREEYDVLD |
| | | KRRGRDPEMGGKPRRKNPQEGLYNELQKDKMAEAYSEIGMKGERRRGKGH |
| | | DGLYQGLSTATKDTYDALHMQALPPR |
| 20 | 1558 | MALPVTALLLPLALLLHAARPDIQMTQSPSSLSASVGDRVTITCRASQGIGND |
| | | LGWYQQKPGKAPKLLIYGASSVQSGVPSRFSGSGSGTDFTLTISSLQPEDFAT |
| | | YYCQQSYSTPITFGQGTRLEIKRTTTPAPRPPTPAPTIASQPLSLRPEACRPAAG |
| | | GAVHTRGLDFACDQVQLVQSGAEVKKPGSSVKVSCKASGGTFSTYAITWVR |
| | | QAPGQGLEWMGGIIPIVGRANYAQKFQGRVTITADESTSTAYMELSSLRSED |
| | | TAVYYCARSGGHDLDYWGQGTLVTVSSGGGGSGGGGSGGGGSGGGGSFW |
| | | VLVVVGGVLACYSLLVTVAFIIFWVRSKRSRLLHSDYMNMTPRRPGPTRKH |
| | | YQPYAPPRDFAAYRSKRGRKKLLYIFKQPFMRPVQTTQEEDGCSCRFPEEEE |
| | | GGCELRVKFSRSADAPAYKQGQNQLYNELNLGRREEYDVLDKRRGRDPEM |
| | | GGKPRRKNPQEGLYNELQKDKMAEAYSEIGMKGERRRGKGHDGLYQGLST |
| | | ATKDTYDALHMQALPPR |
| 21 | 1559 | MALPVTALLLPLALLLHAARPDIQMTQSPSSLSASVGDRVTITCRASQGIGND |
| | | LGWYQQKPGKAPKLLIYGASSVQSGVPSRFSGSGSGTDFTLTISSLQPEDFAT |
| | | YYCQQSYSTPITFGQGTRLEIKRTTTPAPRPPTPAPTIASQPLSLRPEACRPAAG |
| | | GAVHTRGLDFACDQVQLVQSGAEVKKPGSSVKVSCKASGGTFSTYAITWVR |
| | | QAPGQGLEWMGGIIPIVGRANYAQKFQGRVTITADESTSTAYMELSSLRSED |
| | | TAVYYCARSGGHDLDYWGQGTLVTVSSGGGGSGGGGSGGGGSGGGGSFW |
| | | VLVVVGGVLACYSLLVTVAFIIFWVRSKRSRLLHSDYMNMTPRRPGPTRKH |
| | | YQPYAPPRDFAAYRSRVKFSRSADAPAYKQGQNQLYNELNLGRREEYDVLD |
| | | KRRGRDPEMGGKPRRKNPQEGLYNELQKDKMAEAYSEIGMKGERRRGKGH |
| | | DGLYQGLSTATKDTYDALHMQALPPR |
| 22 | 1560 | MALPVTALLLPLALLLHAARPEIVMTQSPATLSVSPGERATLSCRASQSVSSS |
| | | YLAWYQQKPGQAPRLLIYATSTRATGIPARFSGSGSGTEFTLTISSLQSEDFAV |
| | | YYCQQYGSLPLTFGQGTKVEIKRTTTPAPRPPTPAPTIASQPLSLRPEACRPAA |
| | | GGAVHTRGLDFACDEVQLLESGGGLVQPGGSLRLSCAASGFTFSSYGMHWV |
| | | RQAPGKGLEWVSSISGSGDTTYYADSVKGRFTISRDNSKNTLYLQMNSLRAE |
| | | DTAVYYCARDNPYGDYGGSFDYWGQGTLVTVSSGGGGSGGGGSGGGGSG |
| | | GGGSFWVLVVVGGVLACYSLLVTVAFIIFWVKRGRKKLLYIFKQPFMRPVQT |
| | | TQEEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGERR |
| | | RGKGHDGLYQGLSTATKDTYDALHMQALPPR |
| 23 | 1561 | MALPVTALLLPLALLLHAARPEIVMTQSPATLSVSPGERATLSCRASQSVSSS |
| | | YLAWYQQKPGQAPRLLIYATSTRATGIPARFSGSGSGTEFTLTISSLQSEDFAV |
| | | YYCQQYGSLPLTFGQGTKVEIKRTTTPAPRPPTPAPTIASQPLSLRPEACRPAA |
| | | GGAVHTRGLDFACDEVQLLESGGGLVQPGGSLRLSCAASGFTFSSYGMHWV |
| | | RQAPGKGLEWVSSISGSGDTTYYADSVKGRFTISRDNSKNTLYLQMNSLRAE |
| | | DTAVYYCARDNPYGDYGGSFDYWGQGTLVTVSSGGGGSGGGGSGGGGSG |
| | | GGGSFWVLVVVGGVLACYSLLVTVAFIIFWVRSKRSRLLHSDYMNMTPRRP |
| | | GPTRKHYQPYAPPRDFAAYRSKRGRKKLLYIFKQPFMRPVQTTQEEDGCSCR |
| | | FPEEEEGGCELRVKFSRSADAPAYKQGQNQLYNELNLGRREEYDVLDKRRG |
| | | RDPEMGGKPRRKNPQEGLYNELQKDKMAEAYSEIGMKGERRRGKGHDGLY |
| | | QGLSTATKDTYDALHMQALPPR |
| 24 | 1562 | MALPVTALLLPLALLLHAARPEIVMTQSPATLSVSPGERATLSCRASQSVSSS |
| | | YLAWYQQKPGQAPRLLIYATSTRATGIPARFSGSGSGTEFTLTISSLQSEDFAV |
| | | YYCQQYGSLPLTFGQGTKVEIKRTTTPAPRPPTPAPTIASQPLSLRPEACRPAA |
| | | GGAVHTRGLDFACDEVQLLESGGGLVQPGGSLRLSCAASGFTFSSYGMHWV |
| | | RQAPGKGLEWVSSISGSGDTTYYADSVKGRFTISRDNSKNTLYLQMNSLRAE |
| | | DTAVYYCARDNPYGDYGGSFDYWGQGTLVTVSSGGGGSGGGGSGGGGSG |
| | | GGGSFWVLVVVGGVLACYSLLVTVAFIIFWVRSKRSRLLHSDYMNMTPRRP |
| | | GPTRKHYQPYAPPRDFAAYRSRVKFSRSADAPAYKQGQNQLYNELNLGRRE |
| | | EYDVLDKRRGRDPEMGGKPRRKNPQEGLYNELQKDKMAEAYSEIGMKGER |
| | | RRGKGHDGLYQGLSTATKDTYDALHMQALPPR |
| 25 | 1563 | MALPVTALLLPLALLLHAARPDIQMTQSPSSLSASVGDRVTITCRASQGISNN |
| | | LNWYQQKPGKAPKLLIYDASNLETGVPSRFSGSGSGTDFTLTISSLQPEDFAT |
| | | YYCQQANSFPLTFGPGTKVDIKRTTTPAPRPPTPAPTIASQPLSLRPEACRPAA |
| | | GGAVHTRGLDFACDQVQLVQSGAEVKKPGASVKVSCKASGYTFTSYYMHW |
| | | VRQAPGQGLEWMGIIDPSGGSTNYAQKFQGRVTMTRDTSTSTVYMELSSLRS |
| | | EDTAVYYCARDYYGSGSYYGLDYWGRGTLVTVSSGGGGSGGGGSGGGGSG |
| | | GGGSFWVLVVVGGVLACYSLLVTVAFIIFWVKRGRKKLLYIFKQPFMRPVQT |
| | | TQEEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGERR |
| | | RGKGHDGLYQGLSTATKDTYDALHMQALPPR |
| 26 | 1564 | MALPVTALLLPLALLLHAARPDIQMTQSPSSLSASVGDRVTITCRASQGISNN |
| | | LNWYQQKPGKAPKLLIYDASNLETGVPSRFSGSGSGTDFTLTISSLQPEDFAT |
| | | YYCQQANSFPLTFGPGTKVDIKRTTTPAPRPPTPAPTIASQPLSLRPEACRPAA |
| | | GGAVHTRGLDFACDQVQLVQSGAEVKKPGASVKVSCKASGYTFTSYYMHW |
| | | VRQAPGQGLEWMGIIDPSGGSTNYAQKFQGRVTMTRDTSTSTVYMELSSLRS |
| | | EDTAVYYCARDYYGSGSYYGLDYWGRGTLVTVSSGGGGSGGGGSGGGGSG |
| | | GGGSFWVLVVVGGVLACYSLLVTVAFIIFWVRSKRSRLLHSDYMNMTPRRP |
| | | GPTRKHYQPYAPPRDFAAYRSKRGRKKLLYIFKQPFMRPVQTTQEEDGCSCR |
| | | FPEEEEGGCELRVKFSRSADAPAYKQGQNQLYNELNLGRREEYDVLDKRRG |
| | | RDPEMGGKPRRKNPQEGLYNELQKDKMAEAYSEIGMKGERRRGKGHDGLY |
| | | QGLSTATKDTYDALHMQALPPR |
| 27 | 1565 | MALPVTALLLPLALLLHAARPDIQMTQSPSSLSASVGDRVTITCRASQGISNN |
| | | LNWYQQKPGKAPKLLIYDASNLETGVPSRFSGSGSGTDFTLTISSLQPEDFAT |
| | | YYCQQANSFPLTFGPGTKVDIKRTTTPAPRPPTPAPTIASQPLSLRPEACRPAA |
| | | GGAVHTRGLDFACDQVQLVQSGAEVKKPGASVKVSCKASGYTFTSYYMHW |
| | | VRQAPGQGLEWMGIIDPSGGSTNYAQKFQGRVTMTRDTSTSTVYMELSSLRS |
| | | EDTAVYYCARDYYGSGSYYGLDYWGRGTLVTVSSGGGGSGGGGSGGGGSG |
| | | GGGSFWVLVVVGGVLACYSLLVTVAFIIFWVRSKRSRLLHSDYMNMTPRRP |
| | | GPTRKHYQPYAPPRDFAAYRSRVKFSRSADAPAYKQGQNQLYNELNLGRRE |
| | | EYDVLDKRRGRDPEMGGKPRRKNPQEGLYNELQKDKMAEAYSEIGMKGER |
| | | RRGKGHDGLYQGLSTATKDTYDALHMQALPPR |
| 28 | 1566 | MALPVTALLLPLALLLHAARPDIQMTQSPSSLSASVGDRVTITCRASQGISNY |
| | | LAWYQQKPGKAPKLLIYTASTLQSGVPSRFSGSGSGTDFTLTISSLQPEDFAT |
| | | YYCQQSYSTPLTFGGGTKVEIKRTTTPAPRPPTPAPTIASQPLSLRPEACRPAA |
| | | GGAVHTRGLDFACDQVQLVQSGAEVKKPGASVKVSCKASGYSFTSHAISWV |
| | | RQAPGQGLEWMGWIKPNSGDTKYAQKFQGRVTMTRDTSTSTVYMELSSLR |
| | | SEDTAVYYCARGSDDYYGSYYFDYWGQGTLVTVSSGGGGSGGGGSGGGGS |
| | | GGGGSFWVLVVVGGVLACYSLLVTVAFIIFWVKRGRKKLLYIFKQPFMRPV |
| | | QTTQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMKGE |
| | | RRRGKGHDGLYQGLSTATKDTYDALHMQALPPR |
| 29 | 1567 | MALPVTALLLPLALLLHAARPDIQMTQSPSSLSASVGDRVTITCRASQGISNY |
| | | LAWYQQKPGKAPKLLIYTASTLQSGVPSRFSGSGSGTDFTLTISSLQPEDFAT |
| | | YYCQQSYSTPLTFGGGTKVEIKRTTTPAPRPPTPAPTIASQPLSLRPEACRPAA |
| | | GGAVHTRGLDFACDQVQLVQSGAEVKKPGASVKVSCKASGYSFTSHAISWV |
| | | RQAPGQGLEWMGWIKPNSGDTKYAQKFQGRVTMTRDTSTSTVYMELSSLR |
| | | SEDTAVYYCARGSDDYYGSYYFDYWGQGTLVTVSSGGGGSGGGGSGGGGS |
| | | GGGGSFWVLVVVGGVLACYSLLVTVAFIIFWVRSKRSRLLHSDYMNMTPRR |
| | | PGPTRKHYQPYAPPRDFAAYRSKRGRKKLLYIFKQPFMRPVQTTQEEDGCSC |
| | | RFPEEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGHDGL |
| | | YQGLSTATKDTYDALHMQALPPR |
| 30 | 1568 | MALPVTALLLPLALLLHAARPDIQMTQSPSSLSASVGDRVTITCRASQGISNY |
| | | LAWYQQKPGKAPKLLIYTASTLQSGVPSRFSGSGSGTDFTLTISSLQPEDFAT |
| | | YYCQQSYSTPLTFGGGTKVEIKRTTTPAPRPPTPAPTIASQPLSLRPEACRPAA |
| | | GGAVHTRGLDFACDQVQLVQSGAEVKKPGASVKVSCKASGYSFTSHAISWV |
| | | RQAPGQGLEWMGWIKPNSGDTKYAQKFQGRVTMTRDTSTSTVYMELSSLR |
| | | SEDTAVYYCARGSDDYYGSYYFDYWGQGTLVTVSSGGGGSGGGGSGGGGS |
| | | GGGGSFWVLVVVGGVLACYSLLVTVAFIIFWVRSKRSRLLHSDYMNMTPRR |
| | | PGPTRKHYQPYAPPRDFAAYRSRVKFSRSADAPAYKQGQNQLYNELNLGRR |
| | | EEYDVLDKRRGRDPEMGGKPRRKNPQEGLYNELQKDKMAEAYSEIGMKGE |
| | | RRRGKGHDGLYQGLSTATKDTYDALHMQALPPR |
| 31 | 1569 | MALPVTALLLPLALLLHAARPQVQLVQSGAEVKKPGASVKVSCKASGYSFT |
| | | GYYIHWVRQAPGQGLEWMGWINPNSGGTNYAQKFQGRVTMTRDTSTSTVY |
| | | MELSSLRSEDTAVYYCARDQWDGYNSGYFDYWGQGTLVTVSSTTTPAPRPP |
| | | TPAPTIASQPLSLRPEACRPAAGGAVHTRGLDFACDDIQMTQSPSSLSASVGD |
| | | RVTITCRASQTINDWLAWYQQKPGKAPKLLIYSASTLHSGVPSRFSGSGSGTD |
| | | FTLTISSLQPEDFATYYCQQAYSTPWTFGQGTKVEIKRGGGGSGGGGSGGGG |
| | | SGGGGSFWVLVVVGGVLACYSLLVTVAFIIFWVRSKRSRLLHSDYMNMTPR |
| | | RPGPTRKHYQPYAPPRDFAAYRSRVKFSRSADAPAYKQGQNQLYNELNLGR |
| | | REEYDVLDKRRGRDPEMGGKPRRKNPQEGLYNELQKDKMAEAYSEIGMKG |
| | | ERRRGKGHDGLYQGLSTATKDTYDALHMQALPPR |
| 32 | 1570 | MALPVTALLLPLALLLHAARPQVQLVQSGAEVKKPGASVKVSCKASGYSFT |
| | | GYYIHWVRQAPGQGLEWMGWINPNSGGTNYAQKFQGRVTMTRDTSTSTVY |
| | | MELSSLRSEDTAVYYCARDQWDGYNSGYFDYWGQGTLVTVSSTTTPAPRPP |
| | | TPAPTIASQPLSLRPEACRPAAGGAVHTRGLDFACDDIQMTQSPSSLSASVGD |
| | | RVTITCRASQTINDWLAWYQQKPGKAPKLLIYSASTLHSGVPSRFSGSGSGTD |
| | | FTLTISSLQPEDFATYYCQQAYSTPWTFGQGTKVEIKRGGGGSGGGGSGGGG |
| | | SGGGGSFWVLVVVGGVLACYSLLVTVAFIIFWVKRGRKKLLYIFKQPFMRPV |
| | | QTTQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMKGE |
| | | RRRGKGHDGLYQGLSTATKDTYDALHMQALPPR |
| 33 | 1571 | MALPVTALLLPLALLLHAARPQVQLVQSGAEVKKPGASVKVSCKASGYSFT |
| | | GYYIHWVRQAPGQGLEWMGWINPNSGGTNYAQKFQGRVTMTRDTSTSTVY |
| | | MELSSLRSEDTAVYYCARDQWDGYNSGYFDYWGQGTLVTVSSTTTPAPRPP |
| | | TPAPTIASQPLSLRPEACRPAAGGAVHTRGLDFACDDIQMTQSPSSLSASVGD |
| | | RVTITCRASQTINDWLAWYQQKPGKAPKLLIYSASTLHSGVPSRFSGSGSGTD |
| | | FTLTISSLQPEDFATYYCQQAYSTPWTFGQGTKVEIKRGGGGSGGGGSGGGG |
| | | SGGGGSFWVLVVVGGVLACYSLLVTVAFIIFWVRSKRSRLLHSDYMNMTPR |
| | | RPGPTRKHYQPYAPPRDFAAYRSKRGRKKLLYIFKQPFMRPVQTTQEEDGCS |
| | | CRFPEEEEGGCELRVKFSRSADAPAYKQGQNQLYNELNLGRREEYDVLDKR |
| | | RGRDPEMGGKPRRKNPQEGLYNELQKDKMAEAYSEIGMKGERRRGKGHDG |
| | | LYQGLSTATKDTYDALHMQALPPR |
| 34 | 1572 | MALPVTALLLPLALLLHAARPQVQLVQSGAEVKKPGASVKVSCKASGNTFT |
| | | SYGISWVRQAPGQGLEWMGWINPNSGGTKYAQKFQGRVTMTRDTSTSTVY |
| | | MELSSLRSEDTAVYYCARESWFGELYYGMDVWGKGTTVTVSSTTTPAPRPP |
| | | TPAPTIASQPLSLRPEACRPAAGGAVHTRGLDFACDDIVMTQSPLSLPVTPGE |
| | | PASISCRSSQSLLHSNGYNYLDWYLQKPGQSPQLLIYLGSDRASGVPDRESGS |
| | | GSGTDFTLKISRVEAEDVGVYYCMQGLQTPITFGQGTRLEIKRGGGGSGGGG |
| | | SGGGGSGGGGSFWVLVVVGGVLACYSLLVTVAFIIFWVRSKRSRLLHSDYM |
| | | NMTPRRPGPTRKHYQPYAPPRDFAAYRSRVKFSRSADAPAYKQGQNQLYNE |
| | | LNLGRREEYDVLDKRRGRDPEMGGKPRRKNPQEGLYNELQKDKMAEAYSEI |
| | | GMKGERRRGKGHDGLYQGLSTATKDTYDALHMQALPPR |
| 35 | 1573 | MALPVTALLLPLALLLHAARPQVQLVQSGAEVKKPGASVKVSCKASGNTFT |
| | | SYGISWVRQAPGQGLEWMGWINPNSGGTKYAQKFQGRVTMTRDTSTSTVY |
| | | MELSSLRSEDTAVYYCARESWFGELYYGMDVWGKGTTVTVSSTTTPAPRPP |
| | | TPAPTIASQPLSLRPEACRPAAGGAVHTRGLDFACDDIVMTQSPLSLPVTPGE |
| | | PASISCRSSQSLLHSNGYNYLDWYLQKPGQSPQLLIYLGSDRASGVPDRESGS |
| | | GSGTDFTLKISRVEAEDVGVYYCMQGLQTPITFGQGTRLEIKRGGGGSGGGG |
| | | SGGGGSGGGGSFWVLVVVGGVLACYSLLVTVAFIIFWVKRGRKKLLYIFKQP |
| | | FMRPVQTTQEEDGCSCRFPEEEEGGCELRVKFSRSADAPAYKQGQNQLYNEL |
| | | NLGRREEYDVLDKRRGRDPEMGGKPRRKNPQEGLYNELQKDKMAEAYSEI |
| | | GMKGERRRGKGHDGLYQGLSTATKDTYDALHMQALPPR |
| 36 | 1574 | MALPVTALLLPLALLLHAARPQVQLVQSGAEVKKPGASVKVSCKASGNTFT |
| | | SYGISWVRQAPGQGLEWMGWINPNSGGTKYAQKFQGRVTMTRDTSTSTVY |
| | | MELSSLRSEDTAVYYCARESWFGELYYGMDVWGKGTTVTVSSTTTPAPRPP |
| | | TPAPTIASQPLSLRPEACRPAAGGAVHTRGLDFACDDIVMTQSPLSLPVTPGE |
| | | PASISCRSSQSLLHSNGYNYLDWYLQKPGQSPQLLIYLGSDRASGVPDRESGS |
| | | GSGTDFTLKISRVEAEDVGVYYCMQGLQTPITFGQGTRLEIKRGGGGSGGGG |
| | | SGGGGSGGGGSFWVLVVVGGVLACYSLLVTVAFIIFWVRSKRSRLLHSDYM |
| | | NMTPRRPGPTRKHYQPYAPPRDFAAYRSKRGRKKLLYIFKQPFMRPVQTTQE |
| | | EDGCSCRFPEEEEGGCELRVKFSRSADAPAYKQGQNQLYNELNLGRREEYD |
| | | VLDKRRGRDPEMGGKPRRKNPQEGLYNELQKDKMAEAYSEIGMKGERRRG |
| | | KGHDGLYQGLSTATKDTYDALHMQALPPR |
| 37 | 1575 | MALPVTALLLPLALLLHAARPQVQLVQSGAEVKKPGASVKVSCKASGYTFT |
| | | AYYTHWVRQAPGQGLEWMGWMNPNSGHTSYAQKFQGRVTMTRDTSTSTV |
| | | YMELSSLRSEDTAVYYCAREAYDSFDYWGQGTLVTVSSTTTPAPRPPTPAPTI |
| | | ASQPLSLRPEACRPAAGGAVHTRGLDFACDDIQMTQSPSSLSASVGDRVTITC |
| | | RASQSISSYLNWYQQKPGKAPKLLIYEASTLETGVPSRFSGSGSGTDFTLTISS |
| | | LQPEDFATYYCQQANSFPFTFGPGTKVDIKRGGGGSGGGGSGGGGSGGGGSF |
| | | WVLVVVGGVLACYSLLVTVAFIIFWVRSKRSRLLHSDYMNMTPRRPGPTRK |
| | | HYQPYAPPRDFAAYRSRVKFSRSADAPAYKQGQNQLYNELNLGRREEYDVL |
| | | DKRRGRDPEMGGKPRRKNPQEGLYNELQKDKMAEAYSEIGMKGERRRGKG |
| | | HDGLYQGLSTATKDTYDALHMQALPPR |
| 38 | 1576 | MALPVTALLLPLALLLHAARPQVQLVQSGAEVKKPGASVKVSCKASGYTFT |
| | | AYYTHWVRQAPGQGLEWMGWMNPNSGHTSYAQKFQGRVTMTRDTSTSTV |
| | | YMELSSLRSEDTAVYYCAREAYDSFDYWGQGTLVTVSSTTTPAPRPPTPAPTI |
| | | ASQPLSLRPEACRPAAGGAVHTRGLDFACDDIQMTQSPSSLSASVGDRVTITC |
| | | RASQSISSYLNWYQQKPGKAPKLLIYEASTLETGVPSRFSGSGSGTDFTLTISS |
| | | LQPEDFATYYCQQANSFPFTFGPGTKVDIKRGGGGSGGGGSGGGGSGGGGSF |
| | | WVLVVVGGVLACYSLLVTVAFIIFWVKRGRKKLLYIFKQPFMRPVQTTQEED |
| | | GCSCRFPEEEEGGCELRVKFSRSADAPAYKQGQNQLYNELNLGRREEYDVL |
| | | DKRRGRDPEMGGKPRRKNPQEGLYNELQKDKMAEAYSEIGMKGERRRGKG |
| | | HDGLYQGLSTATKDTYDALHMQALPPR |
| 39 | 1577 | MALPVTALLLPLALLLHAARPQVQLVQSGAEVKKPGASVKVSCKASGYTFT |
| | | AYYTHWVRQAPGQGLEWMGWMNPNSGHTSYAQKFQGRVTMTRDTSTSTV |
| | | YMELSSLRSEDTAVYYCAREAYDSFDYWGQGTLVTVSSTTTPAPRPPTPAPTI |
| | | ASQPLSLRPEACRPAAGGAVHTRGLDFACDDIQMTQSPSSLSASVGDRVTITC |
| | | RASQSISSYLNWYQQKPGKAPKLLIYEASTLETGVPSRFSGSGSGTDFTLTISS |
| | | LQPEDFATYYCQQANSFPFTFGPGTKVDIKRGGGGSGGGGSGGGGSGGGGSF |
| | | WVLVVVGGVLACYSLLVTVAFIIFWVRSKRSRLLHSDYMNMTPRRPGPTRK |
| | | HYQPYAPPRDFAAYRSKRGRKKLLYIFKQPFMRPVQTTQEEDGCSCRFPEEE |
| | | EGGCELRVKFSRSADAPAYKQGQNQLYNELNLGRREEYDVLDKRRGRDPE |
| | | MGGKPRRKNPQEGLYNELQKDKMAEAYSEIGMKGERRRGKGHDGLYQGLS |
| | | TATKDTYDALHMQALPPR |
| 40 | 1578 | MALPVTALLLPLALLLHAARPQVQLVQSGAEVKKPGASVKVSCKASGYTFT |
| | | DYYMHWVRQAPGQGLEWMGWINPNSGGTNYAQKFQGRVTMTRDTSTSTV |
| | | YMELSSLRSEDTAVYYCARDSRIAVAASSFDYWGQGTLVTVSSTTTPAPRPP |
| | | TPAPTIASQPLSLRPEACRPAAGGAVHTRGLDFACDDIQMTQSPSSLSASVGD |
| | | RVTITCRASRGINNWLTWYQQKPGKAPKLLIYGASSLQSGVPSRFSGSGSGTD |
| | | FTLTISSLQPEDFATYYCQQSYRIPYTFGQGTKLEIKRGGGGSGGGGSGGGGS |
| | | GGGGSFWVLVVVGGVLACYSLLVTVAFIIFWVRSKRSRLLHSDYMNMTPRR |
| | | PGPTRKHYQPYAPPRDFAAYRSRVKFSRSADAPAYKQGQNQLYNELNLGRR |
| | | EEYDVLDKRRGRDPEMGGKPRRKNPQEGLYNELQKDKMAEAYSEIGMKGE |
| | | RRRGKGHDGLYQGLSTATKDTYDALHMQALPPR |
| 41 | 1579 | MALPVTALLLPLALLLHAARPQVQLVQSGAEVKKPGASVKVSCKASGYTFT |
| | | DYYMHWVRQAPGQGLEWMGWINPNSGGTNYAQKFQGRVTMTRDTSTSTV |
| | | YMELSSLRSEDTAVYYCARDSRIAVAASSFDYWGQGTLVTVSSTTTPAPRPP |
| | | TPAPTIASQPLSLRPEACRPAAGGAVHTRGLDFACDDIQMTQSPSSLSASVGD |
| | | RVTITCRASRGINNWLTWYQQKPGKAPKLLIYGASSLQSGVPSRFSGSGSGTD |
| | | FTLTISSLQPEDFATYYCQQSYRIPYTFGQGTKLEIKRGGGGSGGGGSGGGGS |
| | | GGGGSFWVLVVVGGVLACYSLLVTVAFIIFWVKRGRKKLLYIFKQPFMRPV |
| | | QTTQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMKGE |
| | | RRRGKGHDGLYQGLSTATKDTYDALHMQALPPR |
| 42 | 1580 | MALPVTALLLPLALLLHAARPQVQLVQSGAEVKKPGASVKVSCKASGYTFT |
| | | DYYMHWVRQAPGQGLEWMGWINPNSGGTNYAQKFQGRVTMTRDTSTSTV |
| | | YMELSSLRSEDTAVYYCARDSRIAVAASSFDYWGQGTLVTVSSTTTPAPRPP |
| | | TPAPTIASQPLSLRPEACRPAAGGAVHTRGLDFACDDIQMTQSPSSLSASVGD |
| | | RVTITCRASRGINNWLTWYQQKPGKAPKLLIYGASSLQSGVPSRFSGSGSGTD |
| | | FTLTISSLQPEDFATYYCQQSYRIPYTFGQGTKLEIKRGGGGSGGGGSGGGGS |
| | | GGGGSFWVLVVVGGVLACYSLLVTVAFIIFWVRSKRSRLLHSDYMNMTPRR |
| | | PGPTRKHYQPYAPPRDFAAYRSKRGRKKLLYIFKQPFMRPVQTTQEEDGCSC |
| | | RFPEEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGHDGL |
| | | YQGLSTATKDTYDALHMQALPPR |
| 43 | 1581 | MALPVTALLLPLALLLHAARPQVQLVQSGAEVKKPGASVKVSCKASGYTFT |
| | | GYYMHWVRQAPGQGLEWMGRINPNGGSTTYAQKFQGRVTMTRDTSTSTV |
| | | YMELSSLRSEDTAVYYCARDDFYYYYLDFWGKGTTVTVSSTTTPAPRPPTPA |
| | | PTIASQPLSLRPEACRPAAGGAVHTRGLDFACDDIQMTQSPSSLSASVGDRVT |
| | | ITCRASQSINDWLAWYQQKPGKAPKLLIYAASNLQSGVPSRFSGSGSGTDFTL |
| | | TISSLQPEDFATYYCQQGYSTPPTFGQGTKVEIKRGGGGSGGGGSGGGGSGG |
| | | GGSFWVLVVVGGVLACYSLLVTVAFIIFWVRSKRSRLLHSDYMNMTPRRPG |
| | | PTRKHYQPYAPPRDFAAYRSRVKFSRSADAPAYKQGQNQLYNELNLGRREE |
| | | YDVLDKRRGRDPEMGGKPRRKNPQEGLYNELQKDKMAEAYSEIGMKGERR |
| | | RGKGHDGLYQGLSTATKDTYDALHMQALPPR |
| 44 | 1582 | MALPVTALLLPLALLLHAARPQVQLVQSGAEVKKPGASVKVSCKASGYTFT |
| | | GYYMHWVRQAPGQGLEWMGRINPNGGSTTYAQKFQGRVTMTRDTSTSTV |
| | | YMELSSLRSEDTAVYYCARDDFYYYYLDFWGKGTTVTVSSTTTPAPRPPTPA |
| | | PTIASQPLSLRPEACRPAAGGAVHTRGLDFACDDIQMTQSPSSLSASVGDRVT |
| | | ITCRASQSINDWLAWYQQKPGKAPKLLIYAASNLQSGVPSRFSGSGSGTDFTL |
| | | TISSLQPEDFATYYCQQGYSTPPTFGQGTKVEIKRGGGGSGGGGSGGGGSGG |
| | | GGSFWVLVVVGGVLACYSLLVTVAFIIFWVKRGRKKLLYIFKQPFMRPVQTT |
| | | QEEDGCSCRFPEEEEGGCELRVKFSRSADAPAYKQGQNQLYNELNLGRREEY |
| | | DVLDKRRGRDPEMGGKPRRKNPQEGLYNELQKDKMAEAYSEIGMKGERRR |
| | | GKGHDGLYQGLSTATKDTYDALHMQALPPR |
| 45 | 1583 | MALPVTALLLPLALLLHAARPQVQLVQSGAEVKKPGASVKVSCKASGYTFT |
| | | GYYMHWVRQAPGQGLEWMGRINPNGGSTTYAQKFQGRVTMTRDTSTSTV |
| | | YMELSSLRSEDTAVYYCARDDFYYYYLDFWGKGTTVTVSSTTTPAPRPPTPA |
| | | PTIASQPLSLRPEACRPAAGGAVHTRGLDFACDDIQMTQSPSSLSASVGDRVT |
| | | ITCRASQSINDWLAWYQQKPGKAPKLLIYAASNLQSGVPSRFSGSGSGTDFTL |
| | | TISSLQPEDFATYYCQQGYSTPPTFGQGTKVEIKRGGGGSGGGGSGGGGSGG |
| | | GGSFWVLVVVGGVLACYSLLVTVAFIIFWVRSKRSRLLHSDYMNMTPRRPG |
| | | PTRKHYQPYAPPRDFAAYRSKRGRKKLLYIFKQPFMRPVQTTQEEDGCSCRF |
| | | PEEEEGGCELRVKFSRSADAPAYKQGQNQLYNELNLGRREEYDVLDKRRGR |
| | | DPEMGGKPRRKNPQEGLYNELQKDKMAEAYSEIGMKGERRRGKGHDGLYQ |
| | | GLSTATKDTYDALHMQALPPR |
| 46 | 1584 | MALPVTALLLPLALLLHAARPQVQLVQSGAEVKKPGASVKVSCKASGYTFS |
| | | NYYMHWVRQAPGQGLEWMGWMNPDSGTTGYAQKFQGRVTMTRDTSTST |
| | | VYMELSSLRSEDTAVYYCVRDGTMVQGIFDYWGQGTLVTVSSTTTPAPRPPT |
| | | PAPTIASQPLSLRPEACRPAAGGAVHTRGLDFACDDIQMTQSPSSLSASVGDR |
| | | VTITCRASQSISSWLAWYQQKPGKAPKLLIYGASSLQSGVPSRFSGSGSGTDF |
| | | TLTISSLQPEDFATYYCQQTYRTPLTFGPGTKVDIKRGGGGSGGGGSGGGGS |
| | | GGGGSFWVLVVVGGVLACYSLLVTVAFIIFWVRSKRSRLLHSDYMNMTPRR |
| | | PGPTRKHYQPYAPPRDFAAYRSRVKFSRSADAPAYKQGQNQLYNELNLGRR |
| | | EEYDVLDKRRGRDPEMGGKPRRKNPQEGLYNELQKDKMAEAYSEIGMKGE |
| | | RRRGKGHDGLYQGLSTATKDTYDALHMQALPPR |
| 47 | 1585 | MALPVTALLLPLALLLHAARPQVQLVQSGAEVKKPGASVKVSCKASGYTFS |
| | | NYYMHWVRQAPGQGLEWMGWMNPDSGTTGYAQKFQGRVTMTRDTSTST |
| | | VYMELSSLRSEDTAVYYCVRDGTMVQGIFDYWGQGTLVTVSSTTTPAPRPPT |
| | | PAPTIASQPLSLRPEACRPAAGGAVHTRGLDFACDDIQMTQSPSSLSASVGDR |
| | | VTITCRASQSISSWLAWYQQKPGKAPKLLIYGASSLQSGVPSRFSGSGSGTDF |
| | | TLTISSLQPEDFATYYCQQTYRTPLTFGPGTKVDIKRGGGGSGGGGSGGGGS |
| | | GGGGSFWVLVVVGGVLACYSLLVTVAFIIFWVKRGRKKLLYIFKQPFMRPV |
| | | QTTQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMKGE |
| | | RRRGKGHDGLYQGLSTATKDTYDALHMQALPPR |
| 48 | 1586 | MALPVTALLLPLALLLHAARPQVQLVQSGAEVKKPGASVKVSCKASGYTFS |
| | | NYYMHWVRQAPGQGLEWMGWMNPDSGTTGYAQKFQGRVTMTRDTSTST |
| | | VYMELSSLRSEDTAVYYCVRDGTMVQGIFDYWGQGTLVTVSSTTTPAPRPPT |
| | | PAPTIASQPLSLRPEACRPAAGGAVHTRGLDFACDDIQMTQSPSSLSASVGDR |
| | | VTITCRASQSISSWLAWYQQKPGKAPKLLIYGASSLQSGVPSRFSGSGSGTDF |
| | | TLTISSLQPEDFATYYCQQTYRTPLTFGPGTKVDIKRGGGGSGGGGSGGGGS |
| | | GGGGSFWVLVVVGGVLACYSLLVTVAFIIFWVRSKRSRLLHSDYMNMTPRR |
| | | PGPTRKHYQPYAPPRDFAAYRSKRGRKKLLYIFKQPFMRPVQTTQEEDGCSC |
| | | RFPEEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGHDGL |
| | | YQGLSTATKDTYDALHMQALPPR |
| 49 | 1587 | MALPVTALLLPLALLLHAARPQVQLVQSGAEVKKPGSSVKVSCKASGGTFST |
| | | YAITWVRQAPGQGLEWMGGIIPIVGRANYAQKFQGRVTITADESTSTAYMEL |
| | | SSLRSEDTAVYYCARSGGHDLDYWGQGTLVTVSSTTTPAPRPPTPAPTIASQP |
| | | LSLRPEACRPAAGGAVHTRGLDFACDDIQMTQSPSSLSASVGDRVTITCRASQ |
| | | GIGNDLGWYQQKPGKAPKLLIYGASSVQSGVPSRFSGSGSGTDFTLTISSLQP |
| | | EDFATYYCQQSYSTPITFGQGTRLEIKRGGGGSGGGGSGGGGSGGGGSFWVL |
| | | VVVGGVLACYSLLVTVAFIIFWVKRGRKKLLYIFKQPFMRPVQTTQEEDGCS |
| | | CRFPEEEEGGCELRVKFSRSADAPAYKQGQNQLYNELNLGRREEYDVLDKR |
| | | RGRDPEMGGKPRRKNPQEGLYNELQKDKMAEAYSEIGMKGERRRGKGHDG |
| | | LYQGLSTATKDTYDALHMQALPPR |
| 50 | 1588 | MALPVTALLLPLALLLHAARPQVQLVQSGAEVKKPGSSVKVSCKASGGTFST |
| | | YAITWVRQAPGQGLEWMGGIIPIVGRANYAQKFQGRVTITADESTSTAYMEL |
| | | SSLRSEDTAVYYCARSGGHDLDYWGQGTLVTVSSTTTPAPRPPTPAPTIASQP |
| | | LSLRPEACRPAAGGAVHTRGLDFACDDIQMTQSPSSLSASVGDRVTITCRASQ |
| | | GIGNDLGWYQQKPGKAPKLLIYGASSVQSGVPSRFSGSGSGTDFTLTISSLQP |
| | | EDFATYYCQQSYSTPITFGQGTRLEIKRGGGGSGGGGSGGGGSGGGGSFWVL |
| | | VVVGGVLACYSLLVTVAFIIFWVRSKRSRLLHSDYMNMTPRRPGPTRKHYQP |
| | | YAPPRDFAAYRSKRGRKKLLYIFKQPFMRPVQTTQEEDGCSCRFPEEEEGGC |
| | | ELRVKFSRSADAPAYKQGQNQLYNELNLGRREEYDVLDKRRGRDPEMGGK |
| | | PRRKNPQEGLYNELQKDKMAEAYSEIGMKGERRRGKGHDGLYQGLSTATK |
| | | DTYDALHMQALPPR |
| 51 | 1589 | MALPVTALLLPLALLLHAARPQVQLVQSGAEVKKPGSSVKVSCKASGGTFST |
| | | YAITWVRQAPGQGLEWMGGIIPIVGRANYAQKFQGRVTITADESTSTAYMEL |
| | | SSLRSEDTAVYYCARSGGHDLDYWGQGTLVTVSSTTTPAPRPPTPAPTIASQP |
| | | LSLRPEACRPAAGGAVHTRGLDFACDDIQMTQSPSSLSASVGDRVTITCRASQ |
| | | GIGNDLGWYQQKPGKAPKLLIYGASSVQSGVPSRFSGSGSGTDFTLTISSLQP |
| | | EDFATYYCQQSYSTPITFGQGTRLEIKRGGGGSGGGGSGGGGSGGGGSFWVL |
| | | VVVGGVLACYSLLVTVAFIIFWVRSKRSRLLHSDYMNMTPRRPGPTRKHYQP |
| | | YAPPRDFAAYRSRVKFSRSADAPAYKQGQNQLYNELNLGRREEYDVLDKRR |
| | | GRDPEMGGKPRRKNPQEGLYNELQKDKMAEAYSEIGMKGERRRGKGHDGL |
| | | YQGLSTATKDTYDALHMQALPPR |
| 52 | 1590 | MALPVTALLLPLALLLHAARPEVQLLESGGGLVQPGGSLRLSCAASGFTFSSY |
| | | GMHWVRQAPGKGLEWVSSISGSGDTTYYADSVKGRFTISRDNSKNTLYLQM |
| | | NSLRAEDTAVYYCARDNPYGDYGGSFDYWGQGTLVTVSSTTTPAPRPPTPA |
| | | PTIASQPLSLRPEACRPAAGGAVHTRGLDFACDEIVMTQSPATLSVSPGERAT |
| | | LSCRASQSVSSSYLAWYQQKPGQAPRLLIYATSTRATGIPARFSGSGSGTEFT |
| | | LTISSLQSEDFAVYYCQQYGSLPLTFGQGTKVEIKRGGGGSGGGGSGGGGSG |
| | | GGGSFWVLVVVGGVLACYSLLVTVAFIIFWVKRGRKKLLYIFKQPFMRPVQT |
| | | TQEEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGERR |
| | | RGKGHDGLYQGLSTATKDTYDALHMQALPPR |
| 53 | 1591 | MALPVTALLLPLALLLHAARPEVQLLESGGGLVQPGGSLRLSCAASGFTFSSY |
| | | GMHWVRQAPGKGLEWVSSISGSGDTTYYADSVKGRFTISRDNSKNTLYLQM |
| | | NSLRAEDTAVYYCARDNPYGDYGGSFDYWGQGTLVTVSSTTTPAPRPPTPA |
| | | PTIASQPLSLRPEACRPAAGGAVHTRGLDFACDEIVMTQSPATLSVSPGERAT |
| | | LSCRASQSVSSSYLAWYQQKPGQAPRLLIYATSTRATGIPARFSGSGSGTEFT |
| | | LTISSLQSEDFAVYYCQQYGSLPLTFGQGTKVEIKRGGGGSGGGGSGGGGSG |
| | | GGGSFWVLVVVGGVLACYSLLVTVAFIIFWVRSKRSRLLHSDYMNMTPRRP |
| | | GPTRKHYQPYAPPRDFAAYRSKRGRKKLLYIFKQPFMRPVQTTQEEDGCSCR |
| | | FPEEEEGGCELRVKFSRSADAPAYKQGQNQLYNELNLGRREEYDVLDKRRG |
| | | RDPEMGGKPRRKNPQEGLYNELQKDKMAEAYSEIGMKGERRRGKGHDGLY |
| | | QGLSTATKDTYDALHMQALPPR |
| 54 | 1592 | MALPVTALLLPLALLLHAARPEVQLLESGGGLVQPGGSLRLSCAASGFTFSSY |
| | | GMHWVRQAPGKGLEWVSSISGSGDTTYYADSVKGRFTISRDNSKNTLYLQM |
| | | NSLRAEDTAVYYCARDNPYGDYGGSFDYWGQGTLVTVSSTTTPAPRPPTPA |
| | | PTIASQPLSLRPEACRPAAGGAVHTRGLDFACDEIVMTQSPATLSVSPGERAT |
| | | LSCRASQSVSSSYLAWYQQKPGQAPRLLIYATSTRATGIPARFSGSGSGTEFT |
| | | LTISSLQSEDFAVYYCQQYGSLPLTFGQGTKVEIKRGGGGSGGGGSGGGGSG |
| | | GGGSFWVLVVVGGVLACYSLLVTVAFIIFWVRSKRSRLLHSDYMNMTPRRP |
| | | GPTRKHYQPYAPPRDFAAYRSRVKFSRSADAPAYKQGQNQLYNELNLGRRE |
| | | EYDVLDKRRGRDPEMGGKPRRKNPQEGLYNELQKDKMAEAYSEIGMKGER |
| | | RRGKGHDGLYQGLSTATKDTYDALHMQALPPR |
| 55 | 1593 | MALPVTALLLPLALLLHAARPQVQLVQSGAEVKKPGASVKVSCKASGYTFT |
| | | SYYMHWVRQAPGQGLEWMGIIDPSGGSTNYAQKFQGRVTMTRDTSTSTVY |
| | | MELSSLRSEDTAVYYCARDYYGSGSYYGLDYWGRGTLVTVSSTTTPAPRPPT |
| | | PAPTIASQPLSLRPEACRPAAGGAVHTRGLDFACDDIQMTQSPSSLSASVGDR |
| | | VTITCRASQGISNNLNWYQQKPGKAPKLLIYDASNLETGVPSRFSGSGSGTDF |
| | | TLTISSLQPEDFATYYCQQANSFPLTFGPGTKVDIKRGGGGSGGGGSGGGGSG |
| | | GGGSFWVLVVVGGVLACYSLLVTVAFIIFWVKRGRKKLLYIFKQPFMRPVQT |
| | | TQEEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGERR |
| | | RGKGHDGLYQGLSTATKDTYDALHMQALPPR |
| 56 | 1594 | MALPVTALLLPLALLLHAARPQVQLVQSGAEVKKPGASVKVSCKASGYTFT |
| | | SYYMHWVRQAPGQGLEWMGIIDPSGGSTNYAQKFQGRVTMTRDTSTSTVY |
| | | MELSSLRSEDTAVYYCARDYYGSGSYYGLDYWGRGTLVTVSSTTTPAPRPPT |
| | | PAPTIASQPLSLRPEACRPAAGGAVHTRGLDFACDDIQMTQSPSSLSASVGDR |
| | | VTITCRASQGISNNLNWYQQKPGKAPKLLIYDASNLETGVPSRFSGSGSGTDF |
| | | TLTISSLQPEDFATYYCQQANSFPLTFGPGTKVDIKRGGGGSGGGGSGGGGSG |
| | | GGGSFWVLVVVGGVLACYSLLVTVAFIIFWVRSKRSRLLHSDYMNMTPRRP |
| | | GPTRKHYQPYAPPRDFAAYRSKRGRKKLLYIFKQPFMRPVQTTQEEDGCSCR |
| | | FPEEEEGGCELRVKFSRSADAPAYKQGQNQLYNELNLGRREEYDVLDKRRG |
| | | RDPEMGGKPRRKNPQEGLYNELQKDKMAEAYSEIGMKGERRRGKGHDGLY |
| | | QGLSTATKDTYDALHMQALPPR |
| 57 | 1595 | MALPVTALLLPLALLLHAARPQVQLVQSGAEVKKPGASVKVSCKASGYTFT |
| | | SYYMHWVRQAPGQGLEWMGIIDPSGGSTNYAQKFQGRVTMTRDTSTSTVY |
| | | MELSSLRSEDTAVYYCARDYYGSGSYYGLDYWGRGTLVTVSSTTTPAPRPPT |
| | | PAPTIASQPLSLRPEACRPAAGGAVHTRGLDFACDDIQMTQSPSSLSASVGDR |
| | | VTITCRASQGISNNLNWYQQKPGKAPKLLIYDASNLETGVPSRFSGSGSGTDF |
| | | TLTISSLQPEDFATYYCQQANSFPLTFGPGTKVDIKRGGGGSGGGGSGGGGSG |
| | | GGGSFWVLVVVGGVLACYSLLVTVAFIIFWVRSKRSRLLHSDYMNMTPRRP |
| | | GPTRKHYQPYAPPRDFAAYRSRVKFSRSADAPAYKQGQNQLYNELNLGRRE |
| | | EYDVLDKRRGRDPEMGGKPRRKNPQEGLYNELQKDKMAEAYSEIGMKGER |
| | | RRGKGHDGLYQGLSTATKDTYDALHMQALPPR |
| 58 | 1596 | MALPVTALLLPLALLLHAARPQVQLVQSGAEVKKPGASVKVSCKASGYSFT |
| | | SHAISWVRQAPGQGLEWMGWIKPNSGDTKYAQKFQGRVTMTRDTSTSTVY |
| | | MELSSLRSEDTAVYYCARGSDDYYGSYYFDYWGQGTLVTVSSTTTPAPRPPT |
| | | PAPTIASQPLSLRPEACRPAAGGAVHTRGLDFACDDIQMTQSPSSLSASVGDR |
| | | VTITCRASQGISNYLAWYQQKPGKAPKLLIYTASTLQSGVPSRFSGSGSGTDF |
| | | TLTISSLQPEDFATYYCQQSYSTPLTFGGGTKVEIKRGGGGSGGGGSGGGGSG |
| | | GGGSFWVLVVVGGVLACYSLLVTVAFIIFWVKRGRKKLLYIFKQPFMRPVQT |
| | | TQEEDGCSCRFPEEEEGGCELRVKFSRSADAPAYKQGQNQLYNELNLGRREE |
| | | YDVLDKRRGRDPEMGGKPRRKNPQEGLYNELQKDKMAEAYSEIGMKGERR |
| | | RGKGHDGLYQGLSTATKDTYDALHMQALPPR |
| 59 | 1597 | MALPVTALLLPLALLLHAARPQVQLVQSGAEVKKPGASVKVSCKASGYSFT |
| | | SHAISWVRQAPGQGLEWMGWIKPNSGDTKYAQKFQGRVTMTRDTSTSTVY |
| | | MELSSLRSEDTAVYYCARGSDDYYGSYYFDYWGQGTLVTVSSTTTPAPRPPT |
| | | PAPTIASQPLSLRPEACRPAAGGAVHTRGLDFACDDIQMTQSPSSLSASVGDR |
| | | VTITCRASQGISNYLAWYQQKPGKAPKLLIYTASTLQSGVPSRFSGSGSGTDF |
| | | TLTISSLQPEDFATYYCQQSYSTPLTFGGGTKVEIKRGGGGSGGGGSGGGGSG |
| | | GGGSFWVLVVVGGVLACYSLLVTVAFIIFWVRSKRSRLLHSDYMNMTPRRP |
| | | GPTRKHYQPYAPPRDFAAYRSKRGRKKLLYIFKQPFMRPVQTTQEEDGCSCR |
| | | FPEEEEGGCELRVKFSRSADAPAYKQGQNQLYNELNLGRREEYDVLDKRRG |
| | | RDPEMGGKPRRKNPQEGLYNELQKDKMAEAYSEIGMKGERRRGKGHDGLY |
| | | QGLSTATKDTYDALHMQALPPR |
| 60 | 1598 | MALPVTALLLPLALLLHAARPQVQLVQSGAEVKKPGASVKVSCKASGYSFT |
| | | SHAISWVRQAPGQGLEWMGWIKPNSGDTKYAQKFQGRVTMTRDTSTSTVY |
| | | MELSSLRSEDTAVYYCARGSDDYYGSYYFDYWGQGTLVTVSSTTTPAPRPPT |
| | | PAPTIASQPLSLRPEACRPAAGGAVHTRGLDFACDDIQMTQSPSSLSASVGDR |
| | | VTITCRASQGISNYLAWYQQKPGKAPKLLIYTASTLQSGVPSRFSGSGSGTDF |
| | | TLTISSLQPEDFATYYCQQSYSTPLTFGGGTKVEIKRGGGGSGGGGSGGGGSG |
| | | GGGSFWVLVVVGGVLACYSLLVTVAFIIFWVRSKRSRLLHSDYMNMTPRRP |
| | | GPTRKHYQPYAPPRDFAAYRSRVKFSRSADAPAYKQGQNQLYNELNLGRRE |
| | | EYDVLDKRRGRDPEMGGKPRRKNPQEGLYNELQKDKMAEAYSEIGMKGER |
| | | RRGKGHDGLYQGLSTATKDTYDALHMQALPPR |

Similar CARs comprising scFvs with variations are possible as well.

For example, the CD34 tag may be included in the expression vector along with a P2A sequence (so that it is co-expressed as a discrete protein), or the (GGGGS)₄ linker may be substituted for a (GGGGS)₃, (GGGGS)₂, or (GGGGS)₁ linker. For example, also provided are:
CAR Examples 1a-60a which are identical to those above, except that they are accompanied by the CD34 tag;
CAR Examples 1b-60b which are identical to those above, except that they have a (GGGGS)₃ linker;
CAR Examples 1c-60c which are identical to those above, except that they have a (GGGGS)₃ linker and they are accompanied by the CD34 tag;
CAR Examples 1d-60d which are identical to those above, except that they have a (GGGGS)₂ linker;
CAR Examples 1e-60e which are identical to those above, except that they have a (GGGGS)₂ linker and they are accompanied by the CD34 tag;
CAR Examples 1f-60f which are identical to those above, except that they have a (GGGGS) linker; and
CAR Examples 1g-60g which are identical to those above, except that they have a (GGGGS) linker and they are accompanied by the CD34 tag.

### Example 7: CAR-Bearing Immune Effector Cells

CAR-bearing immune effector cells may be constructed, optionally with a genome editing step to effect deletion or suppression of one or more surface proteins. Such surface proteins many include, for example, those that form part of the TCR complex, which may induce GvHD if the cells are administered to patients in the allogeneic setting, or those that are the target antigen of the CAR, which may induce fratricide if expression of the antigen on CAR-T is not suppressed.

For example, in one protocol, on Day 0, CD4+ CD8+ T cells are thawed in a cell culture media. The required number of cells are centrifuged at 200×g for 10 minutes at room temperature. Supernatant is removed completely, cells resuspended cell culture media (TexMacs) supplemented with IL-7 (10 ng/ml) and IL-15 (10 ng/ml) at concentration of 1×10⁶/ml. T cells are stimulated with Miltenyi research grade TransAct™ (10 μl/ml).

On day 1, the required amount of viral vector comprising CAR is added to the activated cells at the required M.O.I (Multiplicity of Infection). Cells and virus are mixed and placed back in incubator at 37° C.

TABLE 12

| CAR-T | | | | | | |
|---|---|---|---|---|---|---|
| Exam- | | | Stimu- | | | |
| ple | Name | Media | lation | Cas9 p | gRNA | Virus |
| 1 | NTD | TexMacs | T Cell | — | — | — |
| | | | TransA | | | |
| | | | ct ™TM | | | |
| | | | (10 μl/ml) | | | |
| 2 | CART- | TexMacs | T Cell | | | CAR- |
| | CD33 | | TransA | | | CD33 |
| | | | ct ™ | | | |
| | | | (50 μl) | | | |
| 3 | CART- | TexMacs | T Cell | | | CAR- |
| | CD33^{G69} | | TransA | | | CD33^{G69} |
| | | | ct TM | | | |
| | | | (50 μl) | | | |
| 4 | CART- | TexMacs | T Cell | | | CAR- |
| | CD33^{R69} | | TransA | | | CD33^{R69} |
| | | | ct ™ | | | |
| | | | (50 μl) | | | |
| 5 | UCART- | TexMacs | T Cell | 10 μg | 20 μg | CAR- |
| | CD33 | | TransA | | TRAC | CD33 |
| | | | ct ™ | | | |
| | | | (50 μl) | | | |
| 6 | UCART- | TexMacs | T Cell | 10 μg | 20 μg | CAR- |
| | CD33^{G69} | | TransA | | TRAC | CD33^{G69} |
| | | | ct ™ | | | |
| | | | (50 μl) | | | |
| 7 | UCART- | TexMacs | T Cell | 10 μg | 20 μg | CAR- |
| | CD33^{R69} | | TransA | | TRAC | CD33^{R69} |
| | | | ct ™ | | | |
| | | | (50 μl) | | | |
| 8 | CART- | TexMacs | T Cell | | | CAR- |
| | CLL-1 | | TransA | | | CLL-1 |
| | | | ct ™ | | | |
| | | | (50 μl) | | | |
| 9 | CART- | TexMacs | T Cell | | | CAR- |
| | CLL-1^{K244} | | TransA | | | CLL-1^{K244} |
| | | | ct ™ | | | |
| | | | (50 μl) | | | |
| 10 | CART- | TexMacs | T Cell | | | CAR- |
| | CLL-^{1Q244} | | TransA | | | CLL-1^{Q244} |
| | | | ct ™ | | | |
| | | | (50 μl) | | | |
| 11 | UCART- | TexMacs | T Cell | 10 μg | 20 μg | CAR- |
| | CLL-1 | | TransA | | TRAC | CLL-1 |
| | | | ct ™ | | | |
| | | | (50 μl) | | | |
| 12 | UCART- | TexMacs | T Cell | 10 μg | 20 μg | CAR- |
| | CLL-1^{K244} | | TransA | | TRAC | CLL-1^{K244} |
| | | | ct ™ | | | |
| | | | (50 μl) | | | |
| 13 | UCART- | TexMacs | T Cell | 10 μg | 20 μg | CAR- |
| | CLL-1^{Q244} | | TransA | | TRAC | CLL-1^{Q244} |
| | | | ct ™ | | | |
| | | | (50 μl) | | | |

On day 3, activated cells are washed to remove stimulation.

If genome editing is desired, cells are harvested and counted. The required number of cells are centrifuged at 100×g for 10 minutes at room temperature. Supernatant is removed completely, cells resuspended in Electroporation buffer (1 ml) (e.g. Maxcyte EP buffer) and transferred to a microcentrifuge tube, and centrifuged at 100×g for 10 minutes at room temperature. Supernatant is removed completely, and cells then resuspended in electroporation buffer (e.g., MaxCyte EP buffer), at the desired concentration (e.g. 5×10⁷/ml).

Commercially available Cas9 Protein (10 μg) and commercially synthesized gRNA (20 μg) are complexed at room temperature for 10 minutes.

Cells (100 μl) are transferred to the tube containing complexed Cas9/gRNA, gently mixed, and everything transferred into a MaxCyte OC100 cuvette. Electroporation is thereafter commenced using Maxcyte program Expanded T cell 2. After this procedure, the activated cells may be transferred to 10 ml of pre-warmed media and returned to the incubator to expand for an additional 7-12 days.

FACS analysis may be used to show the purity of CAR-transduced cells (CAR expression and target gene deletion).

### Example 8: In Vitro Cell Killing Assay

Target AML cell lines may be obtained from commercially vendors (ATCC). Target expression was confirmed by FACS analysis and target cell genotype obtained through DNA sequencing. Cells were modified to express CBR-GFP (Click beetle luciferase and Green Fluorescent Protein). Jurkat cells (target negative) were engineered to over express either the CD33^{G69} variant or CD33^{R69} variant in conjunction with a CD90.1 marker to enable discrimination by FACS in a target protein independent manor. Target cells were co-incubated with:
CART33^{ARG69} comprising an antigen-recognition domain comprising the V_{H} and V_{L} domains disclosed in polypeptide no. 6, or
CART33^{GLY69} comprising an antigen-recognition domain comprising the V_{H} and V_{L} domains disclosed in polypeptide no. 30; or
positive control, variant nonspecific CART33,
   at a range of effector to target cell ratio ranging from, e.g., E:T 2:1 to E:T 1:32 for 24 hours prior to FACS analysis. Absolute cell counts of viable target cells were quantified by flow cytometry (attune using absolute counts in a defined volume). Percent cytotoxicity is defined as viable targets relative to tumor only controls. Data was analyzed using FlowJo V10.

Results are shown in FIG. 4 and FIG. 5. CART33^{ARG69} effectively kill CD33^{ARG69} targets but not CD33^{GLY69} targets. CART33^{GLY69} effectively kill CD33^{GLY69} targets but not CD33^{ARG69} targets. CART33 kill both CD33^{ARG69} and CD33^{GLY69} targets.

The above assay may be repeated with other CAR cells comprising alternate polypeptides and cells expressing the appropriate targets, and may be varied according to methods known in the art; for example, different ratios of effector to target may be used. It is expected that in further experiments of this type, cells expressing polymorphically selective CARS will kill cells expressing the selected target polymorph.

For example, CART33 will kill CD33+ targets independent of the CD33 genotype (CD33^{R69} or CD33^{G69}). CART-CD33^{G69} is expected to kill CD33^{G69} targets (e.g., HL60, KG1a, or Jurkat CD33^{G69}), but not kill CD33^{R69} targets (e.g., TF1, THP1, or Jurkat CD33^{R69}). CART-CD33^{R69} is expected to kill CD33^{R69} targets (e.g., TF1, THP1 or Jurkat CD33^{R69}), but not kill CD33^{G69} targets (e.g., HL60, KG1a, Jurkat CD33G69).

Similarly, cells expressing polymorphically selective CARS targeting polymorphisms of FLT3 and CLL1 will kill cells expressing the selected target polymorph, and will spare cells expressing the other polymorph. CART-FLT3 will kill FLT3+ targets independent of the FLT3 genotype (FLT3^{T227} or FLT3^{M227}). CART-FLT3^{M227} is expected to kill FLT3M227 targets (e.g., Jurkat FLT3M²²⁷), but not kill FLT3T²²⁷ targets (e.g., Jurkat FLT3T²²⁷). CART-FLT3T²²⁷ is expected to kill FLT3T²²⁷ targets (e.g., Jurkat FLT3T²²⁷), but not kill FLT3M227 targets (e.g., Jurkat FLT3^{M227}). CART-CLL1 will kill CLL1+ targets independent of the CLL1 genotype (CLL1^{K244} or CLL1^{Q244}). CART-CLL1^{Q244} is expected to kill CLL1^{Q244} targets (e.g., Jurkat CLL1^{Q244}), but not kill CLL1^{K244} targets (e.g., Jurkat CLL1^{K244}). CART-CLL1^{K244} is expected to kill CLL1^{K244} targets (e.g., Jurkat CLL1^{K244}), but not kill CLL1244 targets (e.g., Jurkat CLL1^{Q244}).

### Example 9: AML Cell Line Xenograft Model of CAR-T Activity

Six to ten week old immunodeficient NOD.Cg-Prkdcscid Il2rgtm1Wjl Tg(CMV-IL3,CSF2,KITLG)1Eav/MloySzJ (NSG-SGM3) mice may be used in murine patient-derived xenograft experiments. Both male and female mice may be used in experiments and randomly assigned to a treatment group.

Target AML cell lines may be obtained from commercially vendors (ATCC). Target expression is confirmed by FACS analysis and target cell genotype obtained through DNA sequencing. Cells were modified to express CBR-GFP (Click beetle luciferase and Green Fluorescent Protein).

Mice are engrafted with an appropriate amount, e.g., 1×10⁶ cells on day −7 followed by infusion of an appropriate amount, e.g., 2×10⁶ CAR-T cells and appropriate controls on day 0.

For example, a CD33^{G69} AML Cell line, KG1a, may be engrafted into mice and treated with either CD33^{G69} CAR-T cells or CD33^{R69} CAR-T cells, a positive control (CD33 CAR-T cells) or a negative control (e.g., CAR negative T cells).

Tumor burden may be monitored by bioluminescent imaging (BLI) weekly. Mice will be monitored for survival. Bone marrow may be extracted from mice and tumor burden assessed using FACS.

It is expected that CART33 (positive control) will kill CD33+ targets independent of the CD33 genotype (CD33^{R69} or CD33^{G69}), reduce tumor burden, and prolong survival. CART-CD33^{G69} is expected to kill CD33^{G69} targets (e.g., HL60 or KG1a), reduce tumor burden, and prolong survival of mice. CD33^{R69} targets (e.g., TF1, THP1, or Jurkat CD33^{R69}) would not be killed by CART-CD33^{G69} and thus CART-CD33^{G69} would not offer a survival advantage or reduce tumor burden. CART-CD33^{R69} is expected to kill CD33^{R69} targets (e.g., TF1 or THP1), reduce tumor burden, and prolong survival of mice. CART-CD33^{R69} is not expected to kill CD33^{G69} targets (e.g., HL60 or KG1a) and thus CART-CD33^{R69} would not offer a survival advantage or reduce tumor burden in mice bearing CD33^{G69} target cell lines.

### Example 10: AML Cell Line Humanized Xenograft Model of CAR-T Activity

Human CD34+ hematopoietic stem cell-engrafted NOD.Cg-Prkdcscid Il2rgtm1Wjl Tg(CMV-IL3,CSF2,KITLG)1Eav/MloySzJ (CD34+ hu-NSG-SGM3) mice may be used in patient-derived xenograft experiments. Both male and female mice may be used in experiments and randomly assigned to a treatment group.

Mice are bled and the engrafted human cells genotyped using PCR based sequencing to determine the phenotype of the polymorphic target.

Target AML cell lines may be obtained from commercially vendors (ATCC). Target expression is confirmed by FACS analysis and target cell genotype obtained through DNA sequencing. Cells were modified to express CBR-GFP (Click beetle luciferase and Green Fluorescent Protein).

Mice are engrafted with, an appropriate amount, e.g., 1×10⁶ AML cells 8-10 weeks following CD34 cord blood engraftment, followed by infusion of an appropriate amount, e.g., 2×10⁶ CAR-T cells and appropriate controls on day 0.

For example, a CD33^{G69} AML Cell line, KG1a, may be engrafted into humanized CD34+ CD33^{R69} mice and treated with either CD33^{G69} CAR-T cells or CD33^{R69} CAR-T cells, a positive control (CD33 CAR-T cells) or a negative control (e.g., CAR negative T cells).

Tumor burden may be monitored by bioluminescent imaging (BLI) weekly. Mice may be monitored for survival. Bone marrow may be extracted from mice and tumor burden assessed using FACS. CD33 expression on engrafted cord blood derived cells, obtained from the blood, spleen and bone marrow of mice will be analyzed by FACS. Red blood cells are lysed using Red Blood Cell Lysing Buffer (Sigma-Aldrich) and washed with ice cold PBS. Samples were prepared for flow cytometry by re-suspending cells in staining buffer (PBS supplemented with 0.5% bovine serum albumin and 2 mM EOTA) and incubating for 30 min at 4° C. with pre-titrated saturating dilutions of appropriate fluorochrome-labeled monoclonal antibodies. Data may be analyzed using FlowJo V10.

It is expected that CART33 (positive control) will kill CD33+ targets independent of the CD33 genotype (CD33^{R69}+ or CD33^{G69}) and reduce tumor burden, but also lose human engrafted hematopoietic cells. CART-CD33^{G69} is expected to kill CD33^{G69} targets (e.g., HL60 or KG1a) and not kill CD33^{R69} engrafted stem cells, prolonging survival by reducing tumor burden while maintaining human hematopoietic cells. CART-CD33^{R69} is expected to kill CD33^{R69} targets (e.g., TF1, THP) and not kill CD33^{G69} engrafted stem cells, prolonging survival by reducing tumor burden while maintaining human hematopoietic cells. Mice which have the same CD33 variant on both AML and engrafted stem cells would be expected have a reduced tumor burden but fail to maintain human hematopoietic cells.

### Example 11: Patient-Derived Xenograft Model of CAR-T Activity

Six to ten week old immunodeficient NOD.Cg-Prkdcscid Il2rgtm1Wjl Tg(CMV-IL3,CSF2,KITLG)1Eav/MloySzJ (NSG-SGM3) mice may be used in murine patient-derived xenograft experiments. Both male and female mice may be used in experiments and randomly assigned to a treatment group.

Xenografts of human hematologic cancers, e.g. AML, may be obtained from a variety of sources known in the art including, for example, the Public Repository of Xenografts (PRoXe, www.PRoXe.org). Mice are engrafted with an appropriate amount, e.g., 1×10⁶ cells on day −7 followed by infusion of an appropriate amount, e.g., 2×10⁶ CAR-T cells and appropriate controls on day 0.

For example, a CD33^{R69} AML xenograft may be engrafted into mice and treated with either CD33^{G69} CAR-T cells or CD33^{R69} CAR-T cells, or a negative control (e.g., CAR negative T cells).

Peripheral blood and spleens are analyzed by flow cytometry after two weeks, four weeks and six weeks post CAR-T infusion. Red blood cells are lysed using Red Blood Cell Lysing Buffer (Sigma-Aldrich) and washed with ice cold PBS. Samples were prepared for flow cytometry by re-suspending cells in staining buffer (PBS supplemented with 0.5% bovine serum albumin and 2 mM EDTA) and incubating for 30 min at 4° C. with pre-titrated saturating dilutions of appropriate fluorochrome-labeled monoclonal antibodies. Data may be analyzed using FlowJo V10.

It is expected that CD33^{R69} CAR-T would kill engrafted CD33^{R69} AML cells, reducing tumor burden and prolonging survival. It is expected that CD33^{G69} CAR-T would be unable to kill engrafted CD33^{R69} AML cells and would offer no survival advantage or reduction in tumor burden. If the engrafted AML was heterozygous, expressing both CD33^{R69} and CD33^{G69}, both CD33^{G69} CAR-T and CD33^{R69} CAR-T would be effective at killing the engrafted primary AML, prolonging survival of mice.

### Example 12: AML Cell Line In Vitro CAR-NK Activity

Target AML cell lines may be obtained from commercial vendors (ATCC). Target expression was confirmed by FACS analysis and target cell genotype obtained through DNA sequencing.

For example, a CD33^{G69} AML Cell line (such as KG1a or HL60), and CD33^{R69} AML cell lines (such as TF1 or THP1) may be cultured in vitro.

NK cells engineered to express scFv-CARs to CD33^{R69} would then be added to the CD33^{R69} or CD33^{G69} cells in culture for 4-24 hours. After culture, death of the CD33^{R69} cells would be expected to be enhanced, while death of CD33^{G69} cells would be no higher than background killing by unmodified NK cells. scFv-CAR NKs to CD33^{G69} would be expected to kill CD33^{G69} cells but would not be enhanced in killing CD33^{R69} cells. As a positive control, an anti-CD33 CAR could be used, and as a negative control, NK cells alone could be used.

Alternatively, NK cells could be cultured in the presence of CD33^{R69} or CD33^{G69} AML cell lines and in the presence of an antibody with a human IgG1 or IgG3 isotype targeting CD33^{R69}. After co-culture, the death of the AML cell lines would be assessed, and would be expected to be higher for CD33^{R69} AML cells. As a positive control, an anti-CD33 antibody could be used, and as a negative control, NK cells alone could be used.

It is expected that CARNK33 (positive control) will kill CD33+ targets independent of the CD33 genotype of the AML (CD33^{R69} or CD33^{G69}). CARNK-CD33^{G69} is expected to kill CD33^{G69} targets (e.g., HL60 or KG1a). CD33^{R69} targets (e.g., TF1 or THP1) would not be killed by CARNK-CD33^{G69} and thus CARNK-CD33^{G69} would not be enhanced in this in vitro assay. CARNK-CD33^{R69} is expected to kill CD33^{R69} targets (e.g., TF1 or THP1). CARNK-CD33^{R69} is not expected to kill CD33^{G69} targets (e.g., HL60 or KG1a) and thus CARNK-CD33^{R69} would not be enhanced in this in vitro assay.

It is expected that treatment comprising administration of NK cells together with an anti-CD33 antibody (positive control) will kill CD33+ targets independent of the CD33 genotype of the AML (CD33^{R69} or CD33^{G69}). NK cells cultured with an anti-CD33^{G69} antibody are expected to kill CD33^{G69} targets (e.g., HL60 or KG1a). CD33^{R69} targets (e.g., TF1 or THP1) would not be killed by NK cells cultured with an anti-CD33^{G69} antibody and thus NK cells administered with an anti-CD33^{G69} antibody would not increase AML cell death in this assay against CD33^{R69} target cell lines. NK cells administered with an anti-CD33^{R69} antibody are expected to kill CD33^{R69} targets (e.g., TF1 or THP1). NK cells cultured with an anti-CD33^{R69} antibody are not expected to kill CD33^{G69} targets (e.g., HL60 or KG1a) and thus NK cells administered with an anti-CD33^{R69} antibody would not increase AML cell death in this assay against CD33^{G69} target cell lines.

### Example 13: AML Cell Line Xenograft Model of CAR-NK Activity

Six to ten week old immunodeficient NOD.Cg-Prkdcscid Il2rgtm1Wjl Tg(CMV-IL3,CSF2,KITLG)1Eav/MloySzJ (NSG-SGM3) mice may be used in murine patient-derived xenograft experiments. Both male and female mice may be used in experiments and randomly assigned to a treatment group.

Target AML cell lines may be obtained from commercially vendors (ATCC). Target expression was confirmed by FACS analysis and target cell genotype obtained through DNA sequencing. Cells were modified to express CBR-GFP (Click beetle luciferase and Green Fluorescent Protein).

Mice are engrafted with an appropriate amount, e.g., 1×10⁶ cells on day −7 followed by infusion of an appropriate amount, e.g., 5×10⁶ CAR-NK or NK cells and appropriate controls on day 0.

For example, a CD33^{G69} AML Cell line, KG1a, may be engrafted into mice and treated with either CD33^{G69} CAR-NK cells or CD33^{R69} CAR-NK cells, a positive control (CD33 CAR-NK cells) or a negative control (e.g., CAR negative NK cells).

Alternatively, a CD33^{R69} AML Cell line, TF1 may be engrafted into mice and treated with either NK cells co-administered with CD33^{G69} or CD33^{R69}-directed antibodies of the human IgG1 or human IgG3 isotype, a positive control (NK cells with a general anti-CD33 antibody) or a negative control (e.g., NK cells only).

Tumor burden may be monitored by bioluminescent imaging (BLI) weekly and bone. Mice will be monitored for survival. Bone marrow may be extracted from mice and tumor burden assessed using FACS.

It is expected that CARNK33 (positive control) will kill CD33+ targets independent of the CD33 genotype of the AML (CD33^{R69} or CD33^{G69}), reduce tumor burden and prolong survival. CARNK-CD33^{G69} is expected to kill CD33^{G69} targets (e.g., HL60 or KG1a), reduce tumor burden, and prolong survival of mice. CD33^{R69} targets (e.g., TF1 or THP1) would not be killed by CARNK-CD33^{G69} and thus CARNK-CD33^{G69} would not offer a survival advantage or reduce tumor burden. CARNK-CD33^{R69} is expected to kill CD33^{R69} targets (e.g., TF1 or THP1), reduce tumor burden, and prolong survival of mice. CARNK-CD33^{R69} is not expected to kill CD33^{G69} targets (e.g., HL60 or KG1a) and thus CARNK-CD33^{R69} would not offer a survival advantage or reduce tumor burden in mice bearing CD33^{G69} target cell lines.

It is expected that treatment comprising administration of NK cells together with an anti-CD33 antibody (positive control) will kill CD33+ targets independent of the CD33 genotype of the AML (CD33^{R69} or CD33^{G69}), reduce tumor burden, and prolong survival. NK cells administered with an anti-CD33^{G69} antibody is expected to kill CD33^{G69} targets (e.g., HL60 or KG1a), reduce tumor burden, and prolong survival of mice. CD33^{R69} targets (e.g., TF1 or THP1,) would not be killed by NK cells administered with an anti-CD33^{G69} antibody and thus NK cells administered with an anti-CD33^{G69} antibody would not offer a survival advantage or reduce tumor burden. NK cells administered with an anti-CD33^{R69} antibody are expected to kill CD33^{R69} targets (e.g., TF1 or THP1), reduce tumor burden, and prolong survival of mice. NK cells administered with an anti-CD33^{R69} antibody are not expected to kill CD33^{G69} targets (e.g., HL60 or KG1a) and thus NK cells administered with an anti-CD33^{R69} antibody would not offer a survival advantage or reduce tumor burden in mice bearing CD33^{G69} target cell lines.

### Example 14: Antibodies Comprising scFvs

Antibodies may be constructed from the scFvs disclosed herein using methods known in the art. For example, antibodies disclosed herein may be generated from expression cassettes of the form:
I-[(leader)(scFv V_{H})(hCγ1)(hCγ2)(hCγ3)(Cγs)]-|
   in a pFuse IgG1 Fc-fusion protein expression plasmid (e.g., Invivogen) and
   |-[(leader)(scFv V_{L})(hC_{κ/λ})]-|
   in a pFuse IgK Fc-fusion protein expression plasmid (e.g., Invivogen).

Alternatively, an anti-CD33-R69 or anti-CD33-G69 antibody may be generated from an expression cassette of the form:
   |-[(leader)(scFv V_{H})(hCγ1)(hCγ2)(hCγ3)(Cγs)]-[P2A]-[(leader)(scFv V_{L})(C_{κ/λ})]-|.
   In either of the foregoing, Cγs may optionally be part of the hCγ3 domain.

The antibodies may be of various isotypes, the constant domains for which are known in the art. For example, for an IgG1 or IgG4, the sequence components may be as shown in Table 13:

TABLE 13
Human Antibody Fc Components

| | hIgG1 AA | hIgG1 Nucleotide | hIgG4 AA | hIgG4 Nucleotide |
|---|---|---|---|---|
| IL2 | MYRMQLLSCI | atgtacaggatgcaactcctgtcttgcat | MYRMQLLSCI | atgtacaggatgcaactcctgtctt |
| Leader | ALSLALVTNS | tgcactaagtcttgcacttgtcacgaatt | ALSLALVTNS | gcattgcactaagtcttgcacttgtc |
| | SEQ ID | cg | SEQ ID | acgaattcg |
| | NO: 1599 | SEQ ID NO: 1600 | NO: 1601 | SEQ ID NO: 1602 |
| Cy1 | STKGPSVFPLA | tccaccaagggcccatcggtcttcccc | STKGPSVFPL | tccaccaagggcccctccgtgttc |
| | PSSKSTSGGTA | ctggcaccctcctccaagagcacctct | APCSRSTSEST | cccctggccccctgctcccgctcc |
| | ALGCLVKDYF | gggggcacagcggccctgggctgcct | AALGCLVKD | acctccgagtccaccgccgccct |
| | PEPVTVSWNS | ggtcaaggactacttccccgaaccggt | YFPEPVTVSW | gggctgcctggtgaaggactactt |
| | GALTSGVHTF | gacggtgtcgtggaactcaggcgccct | NSGALTSGVH | ccccgagcccgtgaccgtgtcct |
| | PAVLQSSGLY | gaccagcggcgtgcacaccttcccgg | TFPAVLQSSG | ggaactccggcgccctgacctcc |
| | SLSSVVTVPSS | ctgtcctacagtcctcaggactctactcc | LYSLSSVVTV | ggcgtgcacaccttccccgccgt |
| | SLGTQTYICNV | ctcagcagcgtggtgaccgtgccctcc | PSSSLGTKTYT | gctgcagtcctccggcctgtactc |
| | NHKPSNTKVD | agcagcttgggcacccagacctacatc | CNVDHKPSNT | cctgtcctccgtggtgaccgtgcc |
| | KKV | tgcaacgtgaatcacaagcccagcaac | KVDKRV | ctcctcctccctgggcaccaagac |
| | SEQ ID | accaaggtggacaagaaagtt | SEQ ID | ctacacctgcaacgtggaccaca |
| | NO: 1603 | SEQ ID NO: 1604 | NO: 1605 | agccctccaacaccaaggtggac |
| | | | | aagcgcgtg |
| | | | | SEQ ID NO: 1606 |
| Hinge | EPKSCDKTHT | GAGCCCAAATCTTGTGA | SKYGPPCPSCP | tccaaatatggtcccccatgcccat |
| (CH) | CPPCP | CAAAACTCACACATGCC | SEQ ID | catgccca |
| | SEQ ID | CACCGTGCCCA | NO: 1609 | SEQ ID NO: 1610 |
| | NO: 1607 | SEQ ID NO: 1608 | | |
| Cy2 | PELLGGPSVFL | cctgaactcctggggggaccgtcagtc | PEFLGGPSVFL | cctgagttcctggggggaccatca |
| | FPPKPKDTLMI | ttcctcttccccccaaaacccaaggaca | FPPKPKDTLMI | gtcttcctgttccccccaaaaccca |
| | SRTPEVTCVV | ccctcatgatctcccggacccctgaggt | SRTPEVTCVV | aggacactctcatgatctcccgga |
| | VDVSHEDPEV | cacatgcgtggtggtggacgtgagcca | VDVSQEDPEV | cccctgaggtcacgtgcgtggtg |
| | KFNWYVDGV | cgaagaccctgaggtcaagttcaactg | QFNWYVDGV | gtggacgtgagccaggaagacc |
| | EVHNAKTKPR | gtacgtggacggcgtggaggtgcataa | EVHNAKTKPR | ccgaggtccagttcaactggtacg |
| | EEQYNSTYRV | tgccaagacaaagccgcgggaggag | EEQFNSTYRV | tggatggcgtggaggtgcataatg |
| | VSVLTVLHQD | cagtacaacagcacgtaccgtgtggtc | VSVLTVLHQD | ccaagacaaagccgcgggagga |
| | WLNGKEYKC | agcgtcctcaccgtcctgcaccaggac | WLNGKEYKC | gcagttcaacagcacgtaccgtgt |
| | KVSNKALPAPI | tggctgaatggcaaggagtacaagtgc | KVSNKGLPSSI | ggtcagcgtcctcaccgtcctgca |
| | EKTISKAK | aaggtctccaacaaagccctoccagcc | EKTISKAK | ccaggactggctgaacggcaag |
| | SEQ ID | cccatcgagaaaaccatctccaaagcc | SEQ ID | gagtacaagtgcaaggtctccaac |
| | NO: 1611 | aaa | NO: 1613 | aaaggcctcccgtcctccatcgag |
| | | SEQ ID NO: 1612 | | aaaaccatctccaaagccaaa |
| | | | | SEQ ID NO: 1614 |
| Cy3 | QPREPQVYTL | cagccccgagaaccacaggtgtacac | QPREPQVYTL | cagccccgagagccacaggtgta |
| | PPSREEMTKN | cctgcccccatcccgggaggagatga | PPSQEEMTKN | caccctgcccccatcccaggagg |
| | QVSLTCLVKG | ccaagaaccaggtcagcctgacctgcc | QVSLTCLVKG | agatgaccaagaaccaggtcagc |
| | FYPSDIAVEW | tggtcaaaggcttctatcccagcgacat | FYPSDIAVEW | ctgacctgcctggtcaaaggcttct |
| | ESNGQPENNY | cgccgtggagtgggagagcaatgggc | ESNGQPENNY | accccagcgacatcgccgtggag |
| | KTTPPVLDSD | agccggagaacaactacaagaccacg | KTTPPVLDSD | tgggagagcaatgggcagccgg |
| | GSFFLYSKLTV | cctcccgtgctggactccgacggctcct | GSFFLYSRLT | agaacaactacaagaccacgcct |
| | DKSRWQQGN | tcttcctctacagcaagctcaccgtgga | VDKSRWQEG | cccgtgctggactccgacggctc |
| | VFSCSVMHEA | caagagcaggtggcagcaggggaac | NVFSCSVMHE | cttcttcctctacagcaggctaacc |
| | LHNHYTQKSL | gtcttctcatgctccgtgatgcacgagg | ALHNHYTQKS | gtggacaagagcaggtggcagg |
| | SLSP | ctctgcacaaccactacacgcagaaga | LSLSL | aggggaatgtcttctcatgctccgt |
| | SEQ ID | gcctctccctgtctccg | SEQ ID | gatgcatgaggctctgcacaacca |
| | NO: 1615 | SEQ ID NO: 1616 | NO: 1617 | ctacacacagaagagcctctccct |
| | | | | gtctctg |
| | | | | SEQ ID NO: 1618 |
| Secre- | GK | ggtaaatga | GK | ggtaaatga |
| tion | SEQ ID | SEQ ID NO: 1620 | SEQ ID | SEQ ID NO: 1622 |
| (Cs) | NO: 1619 | | NO: 1621 | |
| IgK- | TVAAPSVFIFP | acggtggctgcaccatctgtcttcatctt | TVAAPSVFIFP | acggtggctgcaccatctgtcttca |
| Ck1 | PSDEQLKSGT | cccgccatctgatgagcagttgaaatct | PSDEQLKSGT | tcttcccgccatctgatgagcagtt |
| | ASVVCLLNNF | ggaactgcctctgttgtgtgcctgctga | ASVVCLLNNF | gaaatctggaactgcctctgttgtg |
| | YPREAKVQW | ataacttctatcccagagaggccaaagt | YPREAKVQW | tgcctgctgaataacttctatccca |
| | KVDNALQSGN | acagtggaaggtggataacgccctcca | KVDNALQSG | gagaggccaaagtacagtggaa |
| | SQESVTEQDS | atcgggtaactcccaggagagtgtcac | NSQESVTEQD | ggtggataacgccctccaatcgg |
| | KDSTYSLSSTL | agagcaggacagcaaggacagcacct | SKDSTYSLSST | gtaactcccaggagagtgtcaca |
| | TLSKADYEKH | acagcctcagcagcaccctgacgctga | LTLSKADYEK | gagcaggacagcaaggacagca |
| | KVYACEVTHQ | gcaaagcagactacgagaaacacaaa | HKVYACEVT | cctacagcctcagcagcaccctg |
| | GLSSPVTKSFN | gtctacgcctgcgaagtcacccatcag | HQGLSSPVTK | acgctgagcaaagcagactacga |
| | RGEC | ggcctgagctcgcccgtcacaaagag | SFNRGEC | gaaacacaaagtctacgcctgcg |
| | SEQ ID | cttcaacaggggagagtgttag | SEQ ID | aagtcacccatcagggcctgagct |
| | NO: 1623 | SEQ ID NO: 1624 | NO: 1625 | cgcccgtcacaaagagcttcaac |
| | | | | aggggagagtgttag |
| | | | | SEQ ID NO: 1626 |

The foregoing may be combined with, for example: a V_{H} domain which has a polypeptide sequence of any of SEQ ID NOs 151-175, and/or a V_{L} domain which has a polypeptide sequence of any of SEQ ID NOs 176-200; a V_{H} domain which has a polypeptide sequence of any of SEQ ID NOs 303-319, and/or a V_{L} domain which has a polypeptide sequence of any of SEQ ID NOs 320-336; a V_{H} domain which has a polypeptide sequence of any of SEQ ID NOs 481-504, and/or a V_{L} domain which has a polypeptide sequence of any of SEQ ID NOs 505-528; or a V_{H} domain which has a polypeptide sequence of any of SEQ ID NOs 661-682, or a nucleotide sequence encoding any of SEQ ID NOs 661-682, and/or a V_{L} domain which has a polypeptide sequence of any of SEQ ID NOs 683-704; or a nucleotide sequence encoding any of the foregoing.

Additional antibodies may be constructed from V_{H} and V_{L} domains which are nonselective for a particular polymorphism. For example, the elements in Table 13 may be combined, for example, with a V_{H} domain which has a polypeptide sequence of any of SEQ ID NOs 1035-1089, and/or a V_{L} domain which has a polypeptide sequence of any of SEQ ID NOs 1090-1144; or a V_{H} domain which has a polypeptide sequence of any of SEQ ID NOs 1427-1473, and/or a V_{L} domain which has a polypeptide sequence of any of SEQ ID NOs 1474-1520; or a nucleotide sequence encoding any of the foregoing.

A cloning vector, for example a plasmid, comprising sequence of the foregoing form may be expressed in an appropriate cell line, for example 293F cells; transient transfection is typically sufficient. The 293F cells are grown in IgG free FBS with agitation (e.g., roller bottles), and the supernatant harvested over the course of several (e.g., 5) days. Supernatant is purified using Protein A or G columns and the antibody is recovered using methods known on the art.

The antibody so generated may comprise V_{H} and V_{L} domains as shown below in Table 14. Antibody (mAb) Examples 1-42 target CD33, and 43-88 target CLL-1.

TABLE 14
IgG1 Antibodies

| | | SEQ | | |
|---|---|---|---|---|
| | | ID | | SEQ |
| mAb Ex | IgG1 Heavy Chain | NO | IgG1 Light Chain | ID NO |
| 1 | QVQLVQSGAEVKKPGASVKVSCKASGYS | 1627 | DIQMTQSPSSLSASV | 1715 |
| | FTGYYIHWVRQAPGQGLEWMGWINPNSG | | GDRVTITCRASQTIN | |
| | GTNYAQKFQGRVTMTRDTSTSTVYMELSS | | DWLAWYQQKPGKA | |
| | LRSEDTAVYYCARDQWDGYNSGYFDYW | | PKLLIYSASTLHSGV | |
| | GQGTLVTVSSSTKGPSVFPLAPSSKSTSGG | | PSRFSGSGSGTDFTL | |
| | TAALGCLVKDYFPEPVTVSWNSGALTSGV | | TISSLQPEDFATYYC | |
| | HTFPAVLQSSGLYSLSSVVTVPSSSLGTQT | | QQAYSTPWTFGQGT | |
| | YICNVNHKPSNTKVDKKVDKTHTCPPCPP | | KVEIKRTVAAPSVFI | |
| | ELLGGPSVFLFPPKPKDTLMISRTPEVTCV | | FPPSDEQLKSGTASV | |
| | VVDVSHEDPEVKFNWYVDGVEVHNAKTK | | VCLLNNFYPREAKV | |
| | PREEQYNSTYRVVSVLTVLHQDWLNGKE | | QWKVDNALQSGNS | |
| | YKCKVSNKALPAPIEKTISKAKQPREPQVY | | QESVTEQDSKDSTYS | |
| | TLPPSREEMTKNQVSLTCLVKGFYPSDIAV | | LSSTLTLSKADYEKH | |
| | EWESNGQPENNYKTTPPVLDSDGSFFLYS | | KVYACEVTHQGLSS | |
| | KLTVDKSRWQQGNVFSCSVMHEALHNHY | | PVTKSFNRGEC | |
| | TQKSLSLSPGK | | | |
| 2 | EVQLLESGGGLVQPGGSLRLSCAASGFTFS | 1628 | DIQMTQSPSSLSASV | 1716 |
| | DYYMSWVRQAPGKGLEWVSGISGSGYST | | GDRVTITCRASQSIS | |
| | YYADSVKGRFTISRDNSKNTLYLQMNSLR | | RYLNWYQQKPGKA | |
| | AEDTAVYYCARTFGRGPDWYFDLWGRGT | | PKLLIYTASTLQSGV | |
| | LVTVSSSTKGPSVFPLAPSSKSTSGGTAAL | | PSRFSGSGSGTDFTL | |
| | GCLVKDYFPEPVTVSWNSGALTSGVHTFP | | TISSLQPEDFATYYC | |
| | AVLQSSGLYSLSSVVTVPSSSLGTQTYICN | | QQYDDLPLTFGGGT | |
| | VNHKPSNTKVDKKVDKTHTCPPCPPELLG | | KVEIKRTVAAPSVFI | |
| | GPSVFLFPPKPKDTLMISRTPEVTCVVVDV | | FPPSDEQLKSGTASV | |
| | SHEDPEVKFNWYVDGVEVHNAKTKPREE | | VCLLNNFYPREAKV | |
| | QYNSTYRVVSVLTVLHQDWLNGKEYKCK | | QWKVDNALQSGNS | |
| | VSNKALPAPIEKTISKAKQPREPQVYTLPPS | | QESVTEQDSKDSTYS | |
| | REEMTKNQVSLTCLVKGFYPSDIAVEWES | | LSSTLTLSKADYEKH | |
| | NGQPENNYKTTPPVLDSDGSFFLYSKLTV | | KVYACEVTHQGLSS | |
| | DKSRWQQGNVFSCSVMHEALHNHYTQKS | | PVTKSFNRGEC | |
| | LSLSPGK | | | |
| 3 | EVQLLESGGGLVQPGGSLRLSCAASGFTFS | 1629 | DIQMTQSPSSLSASV | 1717 |
| | NSDMNWVRQAPGKGLEWVSAISGSGGST | | GDRVTITCRASQSISS | |
| | YYADSVKGRFTISRDNSKNTLYLQMNSLR | | YLNWYQQKPGKAP | |
| | AEDTAVYYCARGREDDYGDYVFDYWGQ | | KLLIYGASTLHSGVP | |
| | GTLVTVSSSTKGPSVFPLAPSSKSTSGGTA | | SRFSGSGSGTDFTLTI | |
| | ALGCLVKDYFPEPVTVSWNSGALTSGVHT | | SSLQPEDFATYYCQQ | |
| | FPAVLQSSGLYSLSSVVTVPSSSLGTQTYIC | | SYRIPYTFGQGTKLEI | |
| | NVNHKPSNTKVDKKVDKTHTCPPCPPELL | | KRTVAAPSVFIFPPS | |
| | GGPSVFLFPPKPKDTLMISRTPEVTCVVVD | | DEQLKSGTASVVCL | |
| | VSHEDPEVKFNWYVDGVEVHNAKTKPRE | | LNNFYPREAKVQWK | |
| | EQYNSTYRVVSVLTVLHQDWLNGKEYKC | | VDNALQSGNSQESV | |
| | KVSNKALPAPIEKTISKAKQPREPQVYTLP | | TEQDSKDSTYSLSST | |
| | PSREEMTKNQVSLTCLVKGFYPSDIAVEW | | LTLSKADYEKHKVY | |
| | ESNGQPENNYKTTPPVLDSDGSFFLYSKLT | | ACEVTHQGLSSPVT | |
| | VDKSRWQQGNVFSCSVMHEALHNHYTQ | | KSFNRGEC | |
| | KSLSLSPGK | | | |
| 4 | QVQLVQSGAEVKKPGASVKVSCKASGGT | 1630 | EIVMTQSPATLSVSP | 1718 |
| | FSSYAISWVRQAPGQGLEWMGWINPNSG | | GERATLSCRASQNIN | |
| | NTGYAQKFQGRVTMTRDTSTSTVYMELSS | | SDLAWYQQKPGQAP | |
| | LRSEDTAVYYCAREHGDMDVWGQGTTVT | | RLLIYGASTRATGIP | |
| | VSSSTKGPSVFPLAPSSKSTSGGTAALGCL | | ARFSGSGSGTEFTLTI | |
| | VKDYFPEPVTVSWNSGALTSGVHTFPAVL | | SSLQSEDFAVYYCQ | |
| | QSSGLYSLSSVVTVPSSSLGTQTYICNVNH | | QYDSLPFTFGPGTKV | |
| | KPSNTKVDKKVDKTHTCPPCPPELLGGPS | | DIKRTVAAPSVFIFPP | |
| | VFLFPPKPKDTLMISRTPEVTCVVVDVSHE | | SDEQLKSGTASVVC | |
| | DPEVKFNWYVDGVEVHNAKTKPREEQYN | | LLNNFYPREAKVQW | |
| | STYRVVSVLTVLHQDWLNGKEYKCKVSN | | KVDNALQSGNSQES | |
| | KALPAPIEKTISKAKQPREPQVYTLPPSREE | | VTEQDSKDSTYSLSS | |
| | MTKNQVSLTCLVKGFYPSDIAVEWESNGQ | | TLTLSKADYEKHKV | |
| | PENNYKTTPPVLDSDGSFFLYSKLTVDKSR | | YACEVTHQGLSSPV | |
| | WQQGNVFSCSVMHEALHNHYTQKSLSLS | | TKSFNRGEC | |
| | PGK | | | |
| 5 | QVQLVQSGAEVKKPGASVKVSCKASGNT | 1631 | DIVMTQSPLSLPVTP | 1719 |
| | FTSYGISWVRQAPGQGLEWMGWINPNSG | | GEPASISCRSSQSLLH | |
| | GTKYAQKFQGRVTMTRDTSTSTVYMELSS | | SNGYNYLDWYLQKP | |
| | LRSEDTAVYYCARESWFGELYYGMDVWG | | GQSPQLLIYLGSDRA | |
| | KGTTVTVSSSTKGPSVFPLAPSSKSTSGGT | | SGVPDRFSGSGSGTD | |
| | AALGCLVKDYFPEPVTVSWNSGALTSGVH | | FTLKISRVEAEDVGV | |
| | TFPAVLQSSGLYSLSSVVTVPSSSLGTQTYI | | YYCMQGLQTPITFG | |
| | CNVNHKPSNTKVDKKVDKTHTCPPCPPEL | | QGTRLEIKRTVAAPS | |
| | LGGPSVFLFPPKPKDTLMISRTPEVTCVVV | | VFIFPPSDEQLKSGT | |
| | DVSHEDPEVKFNWYVDGVEVHNAKTKPR | | ASVVCLLNNFYPRE | |
| | EEQYNSTYRVVSVLTVLHQDWLNGKEYK | | AKVQWKVDNALQS | |
| | CKVSNKALPAPIEKTISKAKQPREPQVYTL | | GNSQESVTEQDSKD | |
| | PPSREEMTKNQVSLTCLVKGFYPSDIAVE | | STYSLSSTLTLSKAD | |
| | WESNGQPENNYKTTPPVLDSDGSFFLYSK | | YEKHKVYACEVTHQ | |
| | LTVDKSRWQQGNVFSCSVMHEALHNHYT | | GLSSPVTKSFNRGEC | |
| | QKSLSLSPGK | | | |
| 6 | QVQLVQSGAEVKKPGASVKVSCKASGYT | 1632 | DIQMTQSPSSLSASV | 1720 |
| | FTAYYTHWVRQAPGQGLEWMGWMNPNS | | GDRVTITCRASQSISS | |
| | GHTSYAQKFQGRVTMTRDTSTSTVYMELS | | YLNWYQQKPGKAP | |
| | SLRSEDTAVYYCAREAYDSFDYWGQGTL | | KLLIYEASTLETGVP | |
| | VTVSSSTKGPSVFPLAPSSKSTSGGTAALG | | SRFSGSGSGTDFTLTI | |
| | CLVKDYFPEPVTVSWNSGALTSGVHTFPA | | SSLQPEDFATYYCQQ | |
| | VLQSSGLYSLSSVVTVPSSSLGTQTYICNV | | ANSFPFTFGPGTKVD | |
| | NHKPSNTKVDKKVDKTHTCPPCPPELLGG | | IKRTVAAPSVFIFPPS | |
| | PSVFLFPPKPKDTLMISRTPEVTCVVVDVS | | DEQLKSGTASVVCL | |
| | HEDPEVKFNWYVDGVEVHNAKTKPREEQ | | LNNFYPREAKVQWK | |
| | YNSTYRVVSVLTVLHQDWLNGKEYKCKV | | VDNALQSGNSQESV | |
| | SNKALPAPIEKTISKAKQPREPQVYTLPPSR | | TEQDSKDSTYSLSST | |
| | EEMTKNQVSLTCLVKGFYPSDIAVEWESN | | LTLSKADYEKHKVY | |
| | GQPENNYKTTPPVLDSDGSFFLYSKLTVD | | ACEVTHQGLSSPVT | |
| | KSRWQQGNVFSCSVMHEALHNHYTQKSL | | KSFNRGEC | |
| | SLSPGK | | | |
| 7 | QVQLVQSGAEVKKPGASVKVSCKASGYT | 1633 | DIQMTQSPSSLSASV | 1721 |
| | FTDYYMHWVRQAPGQGLEWMGWINPNS | | GDRVTITCRASRGIN | |
| | GGTNYAQKFQGRVTMTRDTSTSTVYMEL | | NWLTWYQQKPGKA | |
| | SSLRSEDTAVYYCARDSRIAVAASSFDYW | | PKLLIYGASSLQSGV | |
| | GQGTLVTVSSSTKGPSVFPLAPSSKSTSGG | | PSRFSGSGSGTDFTL | |
| | TAALGCLVKDYFPEPVTVSWNSGALTSGV | | TISSLQPEDFATYYC | |
| | HTFPAVLQSSGLYSLSSVVTVPSSSLGTQT | | QQSYRIPYTFGQGTK | |
| | YICNVNHKPSNTKVDKKVDKTHTCPPCPP | | LEIKRTVAAPSVFIFP | |
| | ELLGGPSVFLFPPKPKDTLMISRTPEVTCV | | PSDEQLKSGTASVVC | |
| | VVDVSHEDPEVKFNWYVDGVEVHNAKTK | | LLNNFYPREAKVQW | |
| | PREEQYNSTYRVVSVLTVLHQDWLNGKE | | KVDNALQSGNSQES | |
| | YKCKVSNKALPAPIEKTISKAKQPREPQVY | | VTEQDSKDSTYSLSS | |
| | TLPPSREEMTKNQVSLTCLVKGFYPSDIAV | | TLTLSKADYEKHKV | |
| | EWESNGQPENNYKTTPPVLDSDGSFFLYS | | YACEVTHQGLSSPV | |
| | KLTVDKSRWQQGNVFSCSVMHEALHNHY | | TKSFNRGEC | |
| | TQKSLSLSPGK | | | |
| 8 | EVQLLESGGGLVQPGGSLRLSCAASGFTFS | 1634 | DIQMTQSPSSLSASV | 1722 |
| | SYAMSWVRQAPGKGLEWVSDISGSGSGT | | GDRVTITCRASQSVS | |
| | YYADAVKGRFTISRDNSKNTLYLQMNSLR | | SFLNWYQQKPGKAP | |
| | AEDTAVYYCARPGSDGEFDYWGQGTLVT | | KLLIYAASSLQSGVP | |
| | VSSSTKGPSVFPLAPSSKSTSGGTAALGCL | | SRFSGSGSGTDFTLTI | |
| | VKDYFPEPVTVSWNSGALTSGVHTFPAVL | | SSLQPEDFATYYCQQ | |
| | QSSGLYSLSSVVTVPSSSLGTQTYICNVNH | | SYTTPLTFGQGTKVE | |
| | KPSNTKVDKKVDKTHTCPPCPPELLGGPS | | IKRTVAAPSVFIFPPS | |
| | VFLFPPKPKDTLMISRTPEVTCVVVDVSHE | | DEQLKSGTASVVCL | |
| | DPEVKFNWYVDGVEVHNAKTKPREEQYN | | LNNFYPREAKVQWK | |
| | STYRVVSVLTVLHQDWLNGKEYKCKVSN | | VDNALQSGNSQESV | |
| | KALPAPIEKTISKAKQPREPQVYTLPPSREE | | TEQDSKDSTYSLSST | |
| | MTKNQVSLTCLVKGFYPSDIAVEWESNGQ | | LTLSKADYEKHKVY | |
| | PENNYKTTPPVLDSDGSFFLYSKLTVDKSR | | ACEVTHQGLSSPVT | |
| | WQQGNVFSCSVMHEALHNHYTQKSLSLS | | KSFNRGEC | |
| | PGK | | | |
| 9 | QVQLVQSGAEVKKPGSSVKVSCKASGGTF | 1635 | DIQMTQSPSSLSASV | 1723 |
| | SSDAINWVRQAPGQGLEWMGGFDPEDGE | | GDRVTITCRSSRNIS | |
| | TIYAQKFQGRVTITADESTSTAYMELSSLR | | HWLAWYQQKPGKA | |
| | SEDTAVYYCARGPSGYDFEFDYWGQGTL | | PKLLIYKASSLESGV | |
| | VTVSSSTKGPSVFPLAPSSKSTSGGTAALG | | PSRFSGSGSGTDFTL | |
| | CLVKDYFPEPVTVSWNSGALTSGVHTFPA | | TISSLQPEDFATYYC | |
| | VLQSSGLYSLSSVVTVPSSSLGTQTYICNV | | QQAISFPLTFGGGTK | |
| | NHKPSNTKVDKKVDKTHTCPPCPPELLGG | | VEIKRTVAAPSVFIFP | |
| | PSVFLFPPKPKDTLMISRTPEVTCVVVDVS | | PSDEQLKSGTASVVC | |
| | HEDPEVKFNWYVDGVEVHNAKTKPREEQ | | LLNNFYPREAKVQW | |
| | YNSTYRVVSVLTVLHQDWLNGKEYKCKV | | KVDNALQSGNSQES | |
| | SNKALPAPIEKTISKAKQPREPQVYTLPPSR | | VTEQDSKDSTYSLSS | |
| | EEMTKNQVSLTCLVKGFYPSDIAVEWESN | | TLTLSKADYEKHKV | |
| | GQPENNYKTTPPVLDSDGSFFLYSKLTVD | | YACEVTHQGLSSPV | |
| | KSRWQQGNVFSCSVMHEALHNHYTQKSL | | TKSFNRGEC | |
| | SLSPGK | | | |
| 10 | QVQLVQSGAEVKKPGASVKVSCKASGDT | 1636 | DIVMTQSPDSLAVSL | 1724 |
| | FTTYAISWVRQAPGQGLEWMGWINPNSG | | GERATINCKSSQSVL | |
| | VATYANKFQGRVTMTRDTSTSTVYMELSS | | HSSKNKNYLAWYQ | |
| | LRSEDTAVYYCAREGIVGATDAFDIWGQG | | QKPGQPPKLLIYWAS | |
| | TMVTVSSSTKGPSVFPLAPSSKSTSGGTAA | | TRESGVPDRFSGSGS | |
| | LGCLVKDYFPEPVTVSWNSGALTSGVHTF | | GTDFTLTISSLQAED | |
| | PAVLQSSGLYSLSSVVTVPSSSLGTQTYIC | | VAVYYCQQYFTTPP | |
| | NVNHKPSNTKVDKKVDKTHTCPPCPPELL | | TFGPGTKVDIKRTVA | |
| | GGPSVFLFPPKPKDTLMISRTPEVTCVVVD | | APSVFIFPPSDEQLKS | |
| | VSHEDPEVKFNWYVDGVEVHNAKTKPRE | | GTASVVCLLNNFYP | |
| | EQYNSTYRVVSVLTVLHQDWLNGKEYKC | | REAKVQWKVDNAL | |
| | KVSNKALPAPIEKTISKAKQPREPQVYTLP | | QSGNSQESVTEQDS | |
| | PSREEMTKNQVSLTCLVKGFYPSDIAVEW | | KDSTYSLSSTLTLSK | |
| | ESNGQPENNYKTTPPVLDSDGSFFLYSKLT | | ADYEKHKVYACEVT | |
| | VDKSRWQQGNVFSCSVMHEALHNHYTQ | | HQGLSSPVTKSFNRG | |
| | KSLSLSPGK | | EC | |
| 11 | QVQLVQSGAEVKKPGASVKVSCKASGDT | 1637 | DIQMTQSPSSLSASV | 1725 |
| | FTNHYMHWVRQAPGQGLEWMGWINPNS | | GDRVTITCRASQSLG | |
| | GGTNYAQKFQGRVTMTRDTSTSTVYMEL | | SWLAWYQQKPGKA | |
| | SSLRSEDTAVYYCARDLVPAAVGGYFDY | | PKLLIYAASSLQSGV | |
| | WGQGTLVTVSSSTKGPSVFPLAPSSKSTSG | | PSRFSGSGSGTDFTL | |
| | GTAALGCLVKDYFPEPVTVSWNSGALTSG | | TISSLQPEDFATYYC | |
| | VHTFPAVLQSSGLYSLSSVVTVPSSSLGTQ | | QQANSFPLTFGQGT | |
| | TYICNVNHKPSNTKVDKKVDKTHTCPPCP | | KVEIKRTVAAPSVFI | |
| | PELLGGPSVFLFPPKPKDTLMISRTPEVTCV | | FPPSDEQLKSGTASV | |
| | VVDVSHEDPEVKFNWYVDGVEVHNAKTK | | VCLLNNFYPREAKV | |
| | PREEQYNSTYRVVSVLTVLHQDWLNGKE | | QWKVDNALQSGNS | |
| | YKCKVSNKALPAPIEKTISKAKQPREPQVY | | QESVTEQDSKDSTYS | |
| | TLPPSREEMTKNQVSLTCLVKGFYPSDIAV | | LSSTLTLSKADYEKH | |
| | EWESNGQPENNYKTTPPVLDSDGSFFLYS | | KVYACEVTHQGLSS | |
| | KLTVDKSRWQQGNVFSCSVMHEALHNHY | | PVTKSFNRGEC | |
| | TQKSLSLSPGK | | | |
| 12 | EVQLLESGGGLVQPGGSLRLSCAASGFTFS | 1638 | DIQMTQSPSSLSASV | 1726 |
| | SHWMSWVRQAPGKGLEWVSAISGSGGST | | GDRVTITCQASQDID | |
| | YYADSVKGRFTISRDNSKNTLYLQMNSLR | | NYLNWYQQKPGKA | |
| | AEDTAVYYCARDDNSGSQADWGQGTLVT | | PKLLIYDASNLETGV | |
| | VSSSTKGPSVFPLAPSSKSTSGGTAALGCL | | PSRFSGSGSGTDFTL | |
| | VKDYFPEPVTVSWNSGALTSGVHTFPAVL | | TISSLQPEDFATYYC | |
| | QSSGLYSLSSVVTVPSSSLGTQTYICNVNH | | QQSYSTPLTFGGGTK | |
| | KPSNTKVDKKVDKTHTCPPCPPELLGGPS | | LEIKRTVAAPSVFIFP | |
| | VFLFPPKPKDTLMISRTPEVTCVVVDVSHE | | PSDEQLKSGTASVVC | |
| | DPEVKFNWYVDGVEVHNAKTKPREEQYN | | LLNNFYPREAKVQW | |
| | STYRVVSVLTVLHQDWLNGKEYKCKVSN | | KVDNALQSGNSQES | |
| | KALPAPIEKTISKAKQPREPQVYTLPPSREE | | VTEQDSKDSTYSLSS | |
| | MTKNQVSLTCLVKGFYPSDIAVEWESNGQ | | TLTLSKADYEKHKV | |
| | PENNYKTTPPVLDSDGSFFLYSKLTVDKSR | | YACEVTHQGLSSPV | |
| | WQQGNVFSCSVMHEALHNHYTQKSLSLS | | TKSFNRGEC | |
| | PGK | | | |
| 13 | QVQLVQSGAEVKKPGASVKVSCKASGYS | 1639 | DIQMTQSPSSLSASV | 1727 |
| | FTGYYMHWVRQAPGQGLEWMGWINPNS | | GDRVTITCRASQGIR | |
| | GGTYFAQNFQGRVTMTRDTSTSTVYMELS | | NWLAWYQQKPGKA | |
| | SLRSEDTAVYYCVKDRGDRVVTSYLDYW | | PKLLIYAASSLQSGV | |
| | GQGTLVTVSSSTKGPSVFPLAPSSKSTSGG | | PSRFSGSGSGTDFTL | |
| | TAALGCLVKDYFPEPVTVSWNSGALTSGV | | TISSLQPEDFATYYC | |
| | HTFPAVLQSSGLYSLSSVVTVPSSSLGTQT | | QQSYRTPYTFGQGT | |
| | YICNVNHKPSNTKVDKKVDKTHTCPPCPP | | KLEIKRTVAAPSVFIF | |
| | ELLGGPSVFLFPPKPKDTLMISRTPEVTCV | | PPSDEQLKSGTASVV | |
| | VVDVSHEDPEVKFNWYVDGVEVHNAKTK | | CLLNNFYPREAKVQ | |
| | PREEQYNSTYRVVSVLTVLHQDWLNGKE | | WKVDNALQSGNSQE | |
| | YKCKVSNKALPAPIEKTISKAKQPREPQVY | | SVTEQDSKDSTYSLS | |
| | TLPPSREEMTKNQVSLTCLVKGFYPSDIAV | | STLTLSKADYEKHK | |
| | EWESNGQPENNYKTTPPVLDSDGSFFLYS | | VYACEVTHQGLSSP | |
| | KLTVDKSRWQQGNVFSCSVMHEALHNHY | | VTKSFNRGEC | |
| | TQKSLSLSPGK | | | |
| 14 | QVQLVQSGAEVKKPGASVKVSCKASGYT | 1640 | DIVMTQSPDSLAVSL | 1728 |
| | FTGYYMHWVRQAPGQGLEWMGIINPSGG | | GERATINCKSSQSVL | |
| | STSYAQKFQGRVTMTRDTSTSTVYMELSS | | YSSNNKNYLAWYQ | |
| | LRSEDTAVYYCARAAPYYYDSSGYYSGG | | QKPGQPPKLLIYWAS | |
| | YYFDYWGQGTLVTVSSSTKGPSVFPLAPS | | TRESGVPDRFSGSGS | |
| | SKSTSGGTAALGCLVKDYFPEPVTVSWNS | | GTDFTLTISSLQAED | |
| | GALTSGVHTFPAVLQSSGLYSLSSVVTVPS | | VAVYYCQQYYTTPL | |
| | SSLGTQTYICNVNHKPSNTKVDKKVDKTH | | TFGQGTKLEIKRTVA | |
| | TCPPCPPELLGGPSVFLFPPKPKDTLMISRT | | APSVFIFPPSDEQLKS | |
| | PEVTCVVVDVSHEDPEVKFNWYVDGVEV | | GTASVVCLLNNFYP | |
| | HNAKTKPREEQYNSTYRVVSVLTVLHQD | | REAKVQWKVDNAL | |
| | WLNGKEYKCKVSNKALPAPIEKTISKAKQ | | QSGNSQESVTEQDS | |
| | PREPQVYTLPPSREEMTKNQVSLTCLVKG | | KDSTYSLSSTLTLSK | |
| | FYPSDIAVEWESNGQPENNYKTTPPVLDS | | ADYEKHKVYACEVT | |
| | DGSFFLYSKLTVDKSRWQQGNVFSCSVM | | HQGLSSPVTKSFNRG | |
| | HEALHNHYTQKSLSLSPGK | | EC | |
| 15 | EVQLLESGGGLVQPGGSLRLSCAASGFTFS | 1641 | DIVMTQSPLSLPVTP | 1729 |
| | IYEIHWVRQAPGKGLEWVSAISGSGGSTY | | GEPASISCRSSQSLLH | |
| | YADSVKGRFTISRDNSKNTLYLQMNSLRA | | SNGYNYLDWYLQKP | |
| | EDTAVYYCARSYCGGDCWDYYYYYGMD | | GQSPQLLIYLASNRA | |
| | VWGQGTTVTVSSSTKGPSVFPLAPSSKSTS | | SGVPDRFSGSGSGTD | |
| | GGTAALGCLVKDYFPEPVTVSWNSGALTS | | FTLKISRVEAEDVGV | |
| | GVHTFPAVLQSSGLYSLSSVVTVPSSSLGT | | YYCKQTSHIPLTFGQ | |
| | QTYICNVNHKPSNTKVDKKVDKTHTCPPC | | GTKVEIKRTVAAPSV | |
| | PPELLGGPSVFLFPPKPKDTLMISRTPEVTC | | FIFPPSDEQLKSGTAS | |
| | VVVDVSHEDPEVKFNWYVDGVEVHNAKT | | VVCLLNNFYPREAK | |
| | KPREEQYNSTYRVVSVLTVLHQDWLNGK | | VQWKVDNALQSGN | |
| | EYKCKVSNKALPAPIEKTISKAKQPREPQV | | SQESVTEQDSKDSTY | |
| | YTLPPSREEMTKNQVSLTCLVKGFYPSDIA | | SLSSTLTLSKADYEK | |
| | VEWESNGQPENNYKTTPPVLDSDGSFFLY | | HKVYACEVTHQGLS | |
| | SKLTVDKSRWQQGNVFSCSVMHEALHNH | | SPVTKSFNRGEC | |
| | YTQKSLSLSPGK | | | |
| 16 | EVQLVESGGGLVKPGGSLRLSCAASGFTFS | 1642 | DIVMTQSPLSLPVTP | 1730 |
| | DNSMNWVRQAPGKGLEWVSYISSSGSTIY | | GEPASISCRSSQSLLH | |
| | YADSVKGRFTISRDDSKNTLYLQMNSLKT | | SNGYNYLDWYLQKP | |
| | EDTAVYYCARGRASSWPNWFDPWGQGTL | | GQSPQLLIYSASNLQ | |
| | VTVSSSTKGPSVFPLAPSSKSTSGGTAALG | | SGVPDRFSGSGSGTD | |
| | CLVKDYFPEPVTVSWNSGALTSGVHTFPA | | FTLKISRVEAEDVGV | |
| | VLQSSGLYSLSSVVTVPSSSLGTQTYICNV | | YYCMQALQTPPTFG | |
| | NHKPSNTKVDKKVDKTHTCPPCPPELLGG | | QGTKLEIKRTVAAPS | |
| | PSVFLFPPKPKDTLMISRTPEVTCVVVDVS | | VFIFPPSDEQLKSGT | |
| | HEDPEVKFNWYVDGVEVHNAKTKPREEQ | | ASVVCLLNNFYPRE | |
| | YNSTYRVVSVLTVLHQDWLNGKEYKCKV | | AKVQWKVDNALQS | |
| | SNKALPAPIEKTISKAKQPREPQVYTLPPSR | | GNSQESVTEQDSKD | |
| | EEMTKNQVSLTCLVKGFYPSDIAVEWESN | | STYSLSSTLTLSKAD | |
| | GQPENNYKTTPPVLDSDGSFFLYSKLTVD | | YEKHKVYACEVTHQ | |
| | KSRWQQGNVFSCSVMHEALHNHYTQKSL | | GLSSPVTKSFNRGEC | |
| | SLSPGK | | | |
| 17 | EVQLLESGGGLVQPGGSLRLSCAASGFTFS | 1643 | DIQMTQSPSSLSASV | 1731 |
| | SYAMSWVRQAPGKGLEWVSGISYDSDKI | | GDRVTITCRASQGIS | |
| | GYADAVKGRFTISRDNSKNTLYLQMNSLR | | NNLNWYQQKPGKA | |
| | AEDTAVYYCAREWEGFDYWGQGTLVTVS | | PKLLIYESSTLETGVP | |
| | SSTKGPSVFPLAPSSKSTSGGTAALGCLVK | | SRFSGSGSGTDFTLTI | |
| | DYFPEPVTVSWNSGALTSGVHTFPAVLQS | | SSLQPEDFATYYCQQ | |
| | SGLYSLSSVVTVPSSSLGTQTYICNVNHKP | | SYSAPLTFGGGTKVE | |
| | SNTKVDKKVDKTHTCPPCPPELLGGPSVFL | | IKRTVAAPSVFIFPPS | |
| | FPPKPKDTLMISRTPEVTCVVVDVSHEDPE | | DEQLKSGTASVVCL | |
| | VKFNWYVDGVEVHNAKTKPREEQYNSTY | | LNNFYPREAKVQWK | |
| | RVVSVLTVLHQDWLNGKEYKCKVSNKAL | | VDNALQSGNSQESV | |
| | PAPIEKTISKAKQPREPQVYTLPPSREEMTK | | TEQDSKDSTYSLSST | |
| | NQVSLTCLVKGFYPSDIAVEWESNGQPEN | | LTLSKADYEKHKVY | |
| | NYKTTPPVLDSDGSFFLYSKLTVDKSRWQ | | ACEVTHQGLSSPVT | |
| | QGNVFSCSVMHEALHNHYTQKSLSLSPGK | | KSFNRGEC | |
| 18 | QVQLVQSGAEVKKPGASVKVSCKASGYT | 1644 | DIVMTQSPLSLPVTP | 1732 |
| | FTDHYMHWVRQAPGQGLEWMGWINPNS | | GEPASISCRSSQSLLH | |
| | GGTNYAQKFQGRVTMTRDTSTSTVYMEL | | SNGYNYLDWYLQKP | |
| | SSLRSEDTAVYYCAKDKFGDEGSGWYGD | | GQSPQLLIYLGSNRA | |
| | FQHWGQGTLVTVSSSTKGPSVFPLAPSSKS | | SGVPDRFSGSGSGTD | |
| | TSGGTAALGCLVKDYFPEPVTVSWNSGAL | | FTLKISRVEAEDVGV | |
| | TSGVHTFPAVLQSSGLYSLSSVVTVPSSSL | | YYCMQTLRTPLTFG | |
| | GTQTYICNVNHKPSNTKVDKKVDKTHTCP | | GGTKVEIKRTVAAPS | |
| | PCPPELLGGPSVFLFPPKPKDTLMISRTPEV | | VFIFPPSDEQLKSGT | |
| | TCVVVDVSHEDPEVKFNWYVDGVEVHNA | | ASVVCLLNNFYPRE | |
| | KTKPREEQYNSTYRVVSVLTVLHQDWLN | | AKVQWKVDNALQS | |
| | GKEYKCKVSNKALPAPIEKTISKAKQPREP | | GNSQESVTEQDSKD | |
| | QVYTLPPSREEMTKNQVSLTCLVKGFYPS | | STYSLSSTLTLSKAD | |
| | DIAVEWESNGQPENNYKTTPPVLDSDGSF | | YEKHKVYACEVTHQ | |
| | FLYSKLTVDKSRWQQGNVFSCSVMHEAL | | GLSSPVTKSFNRGEC | |
| | HNHYTQKSLSLSPGK | | | |
| 19 | EVQLLESGGGLVQPGGSLRLSCAASGFTFS | 1645 | DIQMTQSPSSLSASV | 1733 |
| | SYWMHWVRQAPGKGLEWVSGFSGSART | | GDRVTITCRASQNIG | |
| | YYADSVKGRFTISRDNSKNTLYLQMNSLR | | PWLAWYQQKPGKA | |
| | AEDTAVYYCAREWSGFDYWGQGTLVTVS | | PKLLIYDAKDLHPGV | |
| | SSTKGPSVFPLAPSSKSTSGGTAALGCLVK | | PSRFSGSGSGTDFTL | |
| | DYFPEPVTVSWNSGALTSGVHTFPAVLQS | | TISSLQPEDFATYYC | |
| | SGLYSLSSVVTVPSSSLGTQTYICNVNHKP | | QQANTFPMTFGQGT | |
| | SNTKVDKKVDKTHTCPPCPPELLGGPSVFL | | RLEIKRTVAAPSVFIF | |
| | FPPKPKDTLMISRTPEVTCVVVDVSHEDPE | | PPSDEQLKSGTASVV | |
| | VKFNWYVDGVEVHNAKTKPREEQYNSTY | | CLLNNFYPREAKVQ | |
| | RVVSVLTVLHQDWLNGKEYKCKVSNKAL | | WKVDNALQSGNSQE | |
| | PAPIEKTISKAKQPREPQVYTLPPSREEMTK | | SVTEQDSKDSTYSLS | |
| | NQVSLTCLVKGFYPSDIAVEWESNGQPEN | | STLTLSKADYEKHK | |
| | NYKTTPPVLDSDGSFFLYSKLTVDKSRWQ | | VYACEVTHQGLSSP | |
| | QGNVFSCSVMHEALHNHYTQKSLSLSPGK | | VTKSFNRGEC | |
| 20 | QVQLVQSGAEVKKPGASVKVSCKASGYM | 1646 | DIQMTQSPSSLSASV | 1734 |
| | FTGYYIHWVRQAPGQGLEWMGWINPNSG | | GDRVTITCRASQSID | |
| | GTNYAQKFQGRVTMTRDTSTSTVYMELSS | | RWLAWYQQKPGKA | |
| | LRSEDTAVYYCAKDRFGSGNYGYMDVW | | PKLLIYGASSLQSGV | |
| | GKGTTVTVSSSTKGPSVFPLAPSSKSTSGG | | PSRFSGSGSGTDFTL | |
| | TAALGCLVKDYFPEPVTVSWNSGALTSGV | | TISSLQPEDFATYYC | |
| | HTFPAVLQSSGLYSLSSVVTVPSSSLGTQT | | QQSYSTPWTFGQGT | |
| | YICNVNHKPSNTKVDKKVDKTHTCPPCPP | | RLEIKRTVAAPSVFIF | |
| | ELLGGPSVFLFPPKPKDTLMISRTPEVTCV | | PPSDEQLKSGTASVV | |
| | VVDVSHEDPEVKFNWYVDGVEVHNAKTK | | CLLNNFYPREAKVQ | |
| | PREEQYNSTYRVVSVLTVLHQDWLNGKE | | WKVDNALQSGNSQE | |
| | YKCKVSNKALPAPIEKTISKAKQPREPQVY | | SVTEQDSKDSTYSLS | |
| | TLPPSREEMTKNQVSLTCLVKGFYPSDIAV | | STLTLSKADYEKHK | |
| | EWESNGQPENNYKTTPPVLDSDGSFFLYS | | VYACEVTHQGLSSP | |
| | KLTVDKSRWQQGNVFSCSVMHEALHNHY | | VTKSFNRGEC | |
| | TQKSLSLSPGK | | | |
| 21 | EVQLLESGGGLVQPGGSLRLSCAASGFTFS | 1647 | DIQMTQSPSSLSASV | 1735 |
| | SYAMSWVRQAPGKGLEWVSAISGSGGST | | GDRVTITCQASQDIS | |
| | YYADSVKGRFTISRDNSKNTLYLQMNSLR | | NNLNWYQQKPGKA | |
| | AEDTAVYYCARELSHDYGGNSDFDYWGQ | | PKLLIYAASGLQSGV | |
| | GTLVTVSSSTKGPSVFPLAPSSKSTSGGTA | | PSRFSGSGSGTDFTL | |
| | ALGCLVKDYFPEPVTVSWNSGALTSGVHT | | TISSLQPEDFATYYC | |
| | FPAVLQSSGLYSLSSVVTVPSSSLGTQTYIC | | QQANSFPLTFGGGT | |
| | NVNHKPSNTKVDKKVDKTHTCPPCPPELL | | KVEIKRTVAAPSVFI | |
| | GGPSVFLFPPKPKDTLMISRTPEVTCVVVD | | FPPSDEQLKSGTASV | |
| | VSHEDPEVKFNWYVDGVEVHNAKTKPRE | | VCLLNNFYPREAKV | |
| | EQYNSTYRVVSVLTVLHQDWLNGKEYKC | | QWKVDNALQSGNS | |
| | KVSNKALPAPIEKTISKAKQPREPQVYTLP | | QESVTEQDSKDSTYS | |
| | PSREEMTKNQVSLTCLVKGFYPSDIAVEW | | LSSTLTLSKADYEKH | |
| | ESNGQPENNYKTTPPVLDSDGSFFLYSKLT | | KVYACEVTHQGLSS | |
| | VDKSRWQQGNVFSCSVMHEALHNHYTQ | | PVTKSFNRGEC | |
| | KSLSLSPGK | | | |
| 22 | QVQLVQSGAEVKKPGASVKVSCKASGYT | 1648 | DIQMTQSPSSLSASV | 1736 |
| | FTDYYIHWVRQAPGQGLEWMGWINPNSG | | GDRVTITCRASRSIR | |
| | GTNYAQEFQGRVTMTRDTSTSTVYMELSS | | TWLAWYQQKPGKA | |
| | LRSEDTAVYYCARDHRIAVAGSYFDYWG | | PKLLIYAASSLQTGV | |
| | QGTLVTVSSSTKGPSVFPLAPSSKSTSGGT | | PSRFSGSGSGTDFTL | |
| | AALGCLVKDYFPEPVTVSWNSGALTSGVH | | TISSLQPEDFATYYC | |
| | TFPAVLQSSGLYSLSSVVTVPSSSLGTQTYI | | QQSYSTPYTFGQGT | |
| | CNVNHKPSNTKVDKKVDKTHTCPPCPPEL | | KLEIKRTVAAPSVFIF | |
| | LGGPSVFLFPPKPKDTLMISRTPEVTCVVV | | PPSDEQLKSGTASVV | |
| | DVSHEDPEVKFNWYVDGVEVHNAKTKPR | | CLLNNFYPREAKVQ | |
| | EEQYNSTYRVVSVLTVLHQDWLNGKEYK | | WKVDNALQSGNSQE | |
| | CKVSNKALPAPIEKTISKAKQPREPQVYTL | | SVTEQDSKDSTYSLS | |
| | PPSREEMTKNQVSLTCLVKGFYPSDIAVE | | STLTLSKADYEKHK | |
| | WESNGQPENNYKTTPPVLDSDGSFFLYSK | | VYACEVTHQGLSSP | |
| | LTVDKSRWQQGNVFSCSVMHEALHNHYT | | VTKSFNRGEC | |
| | QKSLSLSPGK | | | |
| 23 | QVQLVQSGAEVKKPGASVKVSCKASGYP | 1649 | DIQMTQSPSSLSASV | 1737 |
| | FTAHYIHWVRQAPGQGLEWMGWINPNSG | | GDRVTITCRASQGIN | |
| | GTNYAQKFQGRVTMTRDTSTSTVYMELSS | | NWLAWYQQKPGKA | |
| | LRSEDTAVYYCARDVEMATIGAYWYFDL | | PKLLIYDASNLETGV | |
| | WGRGTLVTVSSSTKGPSVFPLAPSSKSTSG | | PSRFSGSGSGTDFTL | |
| | GTAALGCLVKDYFPEPVTVSWNSGALTSG | | TISSLQPEDFATYYC | |
| | VHTFPAVLQSSGLYSLSSVVTVPSSSLGTQ | | QQANSFPPTFGQGTK | |
| | TYICNVNHKPSNTKVDKKVDKTHTCPPCP | | LEIKRTVAAPSVFIFP | |
| | PELLGGPSVFLFPPKPKDTLMISRTPEVTCV | | PSDEQLKSGTASVVC | |
| | VVDVSHEDPEVKFNWYVDGVEVHNAKTK | | LLNNFYPREAKVQW | |
| | PREEQYNSTYRVVSVLTVLHQDWLNGKE | | KVDNALQSGNSQES | |
| | YKCKVSNKALPAPIEKTISKAKQPREPQVY | | VTEQDSKDSTYSLSS | |
| | TLPPSREEMTKNQVSLTCLVKGFYPSDIAV | | TLTLSKADYEKHKV | |
| | EWESNGQPENNYKTTPPVLDSDGSFFLYS | | YACEVTHQGLSSPV | |
| | KLTVDKSRWQQGNVFSCSVMHEALHNHY | | TKSFNRGEC | |
| | TQKSLSLSPGK | | | |
| 24 | QVQLVQSGAEVKKPGSSVKVSCKASGYSF | 1650 | DIVMTQSPLSLPVTP | 1738 |
| | TSYGISWVRQAPGQGLEWLGWISAYNGN | | GEPASISCRSSQSLLH | |
| | TNYGQSLQGRVTITADESTSTAYMELSSLR | | SNGYNYLDWYLQKP | |
| | SEDTAVYYCARARGAGTFFDYWGQGTLV | | GQSPQLLIYDATNLP | |
| | TVSSSTKGPSVFPLAPSSKSTSGGTAALGC | | TGVPDRFSGSGSGTD | |
| | LVKDYFPEPVTVSWNSGALTSGVHTFPAV | | FTLKISRVEAEDVGV | |
| | LQSSGLYSLSSVVTVPSSSLGTQTYICNVN | | YYCMQALQTPFTFG | |
| | HKPSNTKVDKKVDKTHTCPPCPPELLGGP | | QGTKLEIKRTVAAPS | |
| | SVFLFPPKPKDTLMISRTPEVTCVVVDVSH | | VFIFPPSDEQLKSGT | |
| | EDPEVKFNWYVDGVEVHNAKTKPREEQY | | ASVVCLLNNFYPRE | |
| | NSTYRVVSVLTVLHQDWLNGKEYKCKVS | | AKVQWKVDNALQS | |
| | NKALPAPIEKTISKAKQPREPQVYTLPPSRE | | GNSQESVTEQDSKD | |
| | EMTKNQVSLTCLVKGFYPSDIAVEWESNG | | STYSLSSTLTLSKAD | |
| | QPENNYKTTPPVLDSDGSFFLYSKLTVDKS | | YEKHKVYACEVTHQ | |
| | RWQQGNVFSCSVMHEALHNHYTQKSLSL | | GLSSPVTKSFNRGEC | |
| | SPGK | | | |
| 25 | QVQLVQSGAEVKKPGASVKVSCKASGYT | 1651 | DIQMTQSPSSLSASV | 1739 |
| | FTGYYMHWVRQAPGQGLEWMGRINPNG | | GDRVTITCRASQSIN | |
| | GSTTYAQKFQGRVTMTRDTSTSTVYMELS | | DWLAWYQQKPGKA | |
| | SLRSEDTAVYYCARDDFYYYYLDFWGKG | | PKLLIYAASNLQSGV | |
| | TTVTVSSSTKGPSVFPLAPSSKSTSGGTAA | | PSRFSGSGSGTDFTL | |
| | LGCLVKDYFPEPVTVSWNSGALTSGVHTF | | TISSLQPEDFATYYC | |
| | PAVLQSSGLYSLSSVVTVPSSSLGTQTYIC | | QQGYSTPPTFGQGT | |
| | NVNHKPSNTKVDKKVDKTHTCPPCPPELL | | KVEIKRTVAAPSVFI | |
| | GGPSVFLFPPKPKDTLMISRTPEVTCVVVD | | FPPSDEQLKSGTASV | |
| | VSHEDPEVKFNWYVDGVEVHNAKTKPRE | | VCLLNNFYPREAKV | |
| | EQYNSTYRVVSVLTVLHQDWLNGKEYKC | | QWKVDNALQSGNS | |
| | KVSNKALPAPIEKTISKAKQPREPQVYTLP | | QESVTEQDSKDSTYS | |
| | PSREEMTKNQVSLTCLVKGFYPSDIAVEW | | LSSTLTLSKADYEKH | |
| | ESNGQPENNYKTTPPVLDSDGSFFLYSKLT | | KVYACEVTHQGLSS | |
| | VDKSRWQQGNVFSCSVMHEALHNHYTQ | | PVTKSFNRGEC | |
| | KSLSLSPGK | | | |
| 26 | QVQLVQSGAEVKKPGASVKVSCKASGYT | 1652 | DIQMTQSPSSLSASV | 1740 |
| | FTENEMHWVRQAPGQGLEWMGWMNPNS | | GDRVTITCQASQDIR | |
| | GNTGYAQKFQGRVTMTRDTSTSTVYMEL | | NYLNWYQQKPGKA | |
| | SSLRSEDTAVYYCAREGGDWPYYYMDV | | PKLLIYAASSLQSGV | |
| | WGKGTTVTVSSSTKGPSVFPLAPSSKSTSG | | PSRFSGSGSGTDFTL | |
| | GTAALGCLVKDYFPEPVTVSWNSGALTSG | | TISSLQPEDFATYYC | |
| | VHTFPAVLQSSGLYSLSSVVTVPSSSLGTQ | | QQTSSTPLTFGPGTK | |
| | TYICNVNHKPSNTKVDKKVDKTHTCPPCP | | VDIKRTVAAPSVFIFP | |
| | PELLGGPSVFLFPPKPKDTLMISRTPEVTCV | | PSDEQLKSGTASVVC | |
| | VVDVSHEDPEVKFNWYVDGVEVHNAKTK | | LLNNFYPREAKVQW | |
| | PREEQYNSTYRVVSVLTVLHQDWLNGKE | | KVDNALQSGNSQES | |
| | YKCKVSNKALPAPIEKTISKAKQPREPQVY | | VTEQDSKDSTYSLSS | |
| | TLPPSREEMTKNQVSLTCLVKGFYPSDIAV | | TLTLSKADYEKHKV | |
| | EWESNGQPENNYKTTPPVLDSDGSFFLYS | | YACEVTHQGLSSPV | |
| | KLTVDKSRWQQGNVFSCSVMHEALHNHY | | TKSFNRGEC | |
| | TQKSLSLSPGK | | | |
| 27 | QVQLVQSGAEVKKPGASVKVSCKASGYT | 1653 | DIQMTQSPSSLSASV | 1741 |
| | LTGYYMHWVRQAPGQGLEWMGWMNPSS | | GDRVTITCRASQDIR | |
| | GNTGYAQQFQGRVTMTRDTSTSTVYMEL | | NNLGWYQQKPGKA | |
| | SSLRSEDTAVYYCARASSDRYYYDGVWY | | PKLLIYGASSLQSGV | |
| | FDLWGRGTLVTVSSSTKGPSVFPLAPSSKS | | PSRFSGSGSGTDFTL | |
| | TSGGTAALGCLVKDYFPEPVTVSWNSGAL | | TISSLQPEDFATYYC | |
| | TSGVHTFPAVLQSSGLYSLSSVVTVPSSSL | | QQTYSSPPTFGQGTK | |
| | GTQTYICNVNHKPSNTKVDKKVDKTHTCP | | LEIKRTVAAPSVFIFP | |
| | PCPPELLGGPSVFLFPPKPKDTLMISRTPEV | | PSDEQLKSGTASVVC | |
| | TCVVVDVSHEDPEVKFNWYVDGVEVHNA | | LLNNFYPREAKVQW | |
| | KTKPREEQYNSTYRVVSVLTVLHQDWLN | | KVDNALQSGNSQES | |
| | GKEYKCKVSNKALPAPIEKTISKAKQPREP | | VTEQDSKDSTYSLSS | |
| | QVYTLPPSREEMTKNQVSLTCLVKGFYPS | | TLTLSKADYEKHKV | |
| | DIAVEWESNGQPENNYKTTPPVLDSDGSF | | YACEVTHQGLSSPV | |
| | FLYSKLTVDKSRWQQGNVFSCSVMHEAL | | TKSFNRGEC | |
| | HNHYTQKSLSLSPGK | | | |
| 28 | EVQLLESGGGLVQPGGSLRLSCAASGFTFS | 1654 | DIQMTQSPSSLSASV | 1742 |
| | TYAMHWVRQAPGKGLEWVSAISGSGGST | | GDRVTITCRASQGID | |
| | YYADSVKGRFTISRDNSKNTLYLQMNSLR | | NYLAWYQQKPGKA | |
| | AEDTAVYYCARDGYGDYPFDYWGQGTL | | PKLLIYQASTLESGV | |
| | VTVSSSTKGPSVFPLAPSSKSTSGGTAALG | | PSRFSGSGSGTDFTL | |
| | CLVKDYFPEPVTVSWNSGALTSGVHTFPA | | TISSLQPEDFATYYC | |
| | VLQSSGLYSLSSVVTVPSSSLGTQTYICNV | | QQSYSIPWTFGQGTK | |
| | NHKPSNTKVDKKVDKTHTCPPCPPELLGG | | VEIKRTVAAPSVFIFP | |
| | PSVFLFPPKPKDTLMISRTPEVTCVVVDVS | | PSDEQLKSGTASVVC | |
| | HEDPEVKFNWYVDGVEVHNAKTKPREEQ | | LLNNFYPREAKVQW | |
| | YNSTYRVVSVLTVLHQDWLNGKEYKCKV | | KVDNALQSGNSQES | |
| | SNKALPAPIEKTISKAKQPREPQVYTLPPSR | | VTEQDSKDSTYSLSS | |
| | EEMTKNQVSLTCLVKGFYPSDIAVEWESN | | TLTLSKADYEKHKV | |
| | GQPENNYKTTPPVLDSDGSFFLYSKLTVD | | YACEVTHQGLSSPV | |
| | KSRWQQGNVFSCSVMHEALHNHYTQKSL | | TKSFNRGEC | |
| | SLSPGK | | | |
| 29 | QVQLVQSGAEVKKPGASVKVSCKASGYT | 1655 | DIQMTQSPSSLSASV | 1743 |
| | FTGYYLHWVRQAPGQGLEWMGVINVRRG | | GDRVTITCRASQSIS | |
| | STRYAQNFQGRVTMTRDTSTSTVYMELSS | | RWLAWYQQKPGKA | |
| | LRSEDTAVYYCARVSGSYYQPWGQGTLV | | PKLLIYDASNLETGV | |
| | TVSSSTKGPSVFPLAPSSKSTSGGTAALGC | | PSRFSGSGSGTDFTL | |
| | LVKDYFPEPVTVSWNSGALTSGVHTFPAV | | TISSLQPEDFATYYC | |
| | LQSSGLYSLSSVVTVPSSSLGTQTYICNVN | | QQGNSFPPIFGGGTK | |
| | HKPSNTKVDKKVDKTHTCPPCPPELLGGP | | VEIKRTVAAPSVFIFP | |
| | SVFLFPPKPKDTLMISRTPEVTCVVVDVSH | | PSDEQLKSGTASVVC | |
| | EDPEVKFNWYVDGVEVHNAKTKPREEQY | | LLNNFYPREAKVQW | |
| | NSTYRVVSVLTVLHQDWLNGKEYKCKVS | | KVDNALQSGNSQES | |
| | NKALPAPIEKTISKAKQPREPQVYTLPPSRE | | VTEQDSKDSTYSLSS | |
| | EMTKNQVSLTCLVKGFYPSDIAVEWESNG | | TLTLSKADYEKHKV | |
| | QPENNYKTTPPVLDSDGSFFLYSKLTVDKS | | YACEVTHQGLSSPV | |
| | RWQQGNVFSCSVMHEALHNHYTQKSLSL | | TKSFNRGEC | |
| | SPGK | | | |
| 30 | QVQLVQSGAEVKKPGASVKVSCKASGYT | 1656 | DIQMTQSPSSLSASV | 1744 |
| | FSNYYMHWVRQAPGQGLEWMGWMNPD | | GDRVTITCRASQSISS | |
| | SGTTGYAQKFQGRVTMTRDTSTSTVYME | | WLAWYQQKPGKAP | |
| | LSSLRSEDTAVYYCVRDGTMVQGIFDYW | | KLLIYGASSLQSGVP | |
| | GQGTLVTVSSSTKGPSVFPLAPSSKSTSGG | | SRFSGSGSGTDFTLTI | |
| | TAALGCLVKDYFPEPVTVSWNSGALTSGV | | SSLQPEDFATYYCQQ | |
| | HTFPAVLQSSGLYSLSSVVTVPSSSLGTQT | | TYRTPLTFGPGTKVD | |
| | YICNVNHKPSNTKVDKKVDKTHTCPPCPP | | IKRTVAAPSVFIFPPS | |
| | ELLGGPSVFLFPPKPKDTLMISRTPEVTCV | | DEQLKSGTASVVCL | |
| | VVDVSHEDPEVKFNWYVDGVEVHNAKTK | | LNNFYPREAKVQWK | |
| | PREEQYNSTYRVVSVLTVLHQDWLNGKE | | VDNALQSGNSQESV | |
| | YKCKVSNKALPAPIEKTISKAKQPREPQVY | | TEQDSKDSTYSLSST | |
| | TLPPSREEMTKNQVSLTCLVKGFYPSDIAV | | LTLSKADYEKHKVY | |
| | EWESNGQPENNYKTTPPVLDSDGSFFLYS | | ACEVTHQGLSSPVT | |
| | KLTVDKSRWQQGNVFSCSVMHEALHNHY | | KSFNRGEC | |
| | TQKSLSLSPGK | | | |
| 31 | QVQLVQSGAEVKKPGSSVKVSCKASGGTF | 1657 | DIQMTQSPSSLSASV | 1745 |
| | STYAITWVRQAPGQGLEWMGGIIPIVGRA | | GDRVTITCRASQGIG | |
| | NYAQKFQGRVTITADESTSTAYMELSSLRS | | NDLGWYQQKPGKA | |
| | EDTAVYYCARSGGHDLDYWGQGTLVTVS | | PKLLIYGASSVQSGV | |
| | SSTKGPSVFPLAPSSKSTSGGTAALGCLVK | | PSRFSGSGSGTDFTL | |
| | DYFPEPVTVSWNSGALTSGVHTFPAVLQS | | TISSLQPEDFATYYC | |
| | SGLYSLSSVVTVPSSSLGTQTYICNVNHKP | | QQSYSTPITFGQGTR | |
| | SNTKVDKKVDKTHTCPPCPPELLGGPSVFL | | LEIKRTVAAPSVFIFP | |
| | FPPKPKDTLMISRTPEVTCVVVDVSHEDPE | | PSDEQLKSGTASVVC | |
| | VKFNWYVDGVEVHNAKTKPREEQYNSTY | | LLNNFYPREAKVQW | |
| | RVVSVLTVLHQDWLNGKEYKCKVSNKAL | | KVDNALQSGNSQES | |
| | PAPIEKTISKAKQPREPQVYTLPPSREEMTK | | VTEQDSKDSTYSLSS | |
| | NQVSLTCLVKGFYPSDIAVEWESNGQPEN | | TLTLSKADYEKHKV | |
| | NYKTTPPVLDSDGSFFLYSKLTVDKSRWQ | | YACEVTHQGLSSPV | |
| | QGNVFSCSVMHEALHNHYTQKSLSLSPGK | | TKSFNRGEC | |
| 32 | EVQLLESGGGLVQPGGSLRLSCAASGFTFS | 1658 | EIVMTQSPATLSVSP | 1746 |
| | SYGMHWVRQAPGKGLEWVSSISGSGDTT | | GERATLSCRASQSVS | |
| | YYADSVKGRFTISRDNSKNTLYLQMNSLR | | SSYLAWYQQKPGQA | |
| | AEDTAVYYCARDNPYGDYGGSFDYWGQ | | PRLLIYATSTRATGIP | |
| | GTLVTVSSSTKGPSVFPLAPSSKSTSGGTA | | ARFSGSGSGTEFTLTI | |
| | ALGCLVKDYFPEPVTVSWNSGALTSGVHT | | SSLQSEDFAVYYCQ | |
| | FPAVLQSSGLYSLSSVVTVPSSSLGTQTYIC | | QYGSLPLTFGQGTK | |
| | NVNHKPSNTKVDKKVDKTHTCPPCPPELL | | VEIKRTVAAPSVFIFP | |
| | GGPSVFLFPPKPKDTLMISRTPEVTCVVVD | | PSDEQLKSGTASVVC | |
| | VSHEDPEVKFNWYVDGVEVHNAKTKPRE | | LLNNFYPREAKVQW | |
| | EQYNSTYRVVSVLTVLHQDWLNGKEYKC | | KVDNALQSGNSQES | |
| | KVSNKALPAPIEKTISKAKQPREPQVYTLP | | VTEQDSKDSTYSLSS | |
| | PSREEMTKNQVSLTCLVKGFYPSDIAVEW | | TLTLSKADYEKHKV | |
| | ESNGQPENNYKTTPPVLDSDGSFFLYSKLT | | YACEVTHQGLSSPV | |
| | VDKSRWQQGNVFSCSVMHEALHNHYTQ | | TKSFNRGEC | |
| | KSLSLSPGK | | | |
| 33 | QVQLVQSGAEVKKPGASVKVSCKASGYT | 1659 | DIQMTQSPSSLSASV | 1747 |
| | FTSYYMHWVRQAPGQGLEWMGIIDPSGG | | GDRVTITCRASQGIS | |
| | STNYAQKFQGRVTMTRDTSTSTVYMELSS | | NNLNWYQQKPGKA | |
| | LRSEDTAVYYCARDYYGSGSYYGLDYWG | | PKLLIYDASNLETGV | |
| | RGTLVTVSSSTKGPSVFPLAPSSKSTSGGT | | PSRFSGSGSGTDFTL | |
| | AALGCLVKDYFPEPVTVSWNSGALTSGVH | | TISSLQPEDFATYYC | |
| | TFPAVLQSSGLYSLSSVVTVPSSSLGTQTYI | | QQANSFPLTFGPGTK | |
| | CNVNHKPSNTKVDKKVDKTHTCPPCPPEL | | VDIKRTVAAPSVFIFP | |
| | LGGPSVFLFPPKPKDTLMISRTPEVTCVVV | | PSDEQLKSGTASVVC | |
| | DVSHEDPEVKFNWYVDGVEVHNAKTKPR | | LLNNFYPREAKVQW | |
| | EEQYNSTYRVVSVLTVLHQDWLNGKEYK | | KVDNALQSGNSQES | |
| | CKVSNKALPAPIEKTISKAKQPREPQVYTL | | VTEQDSKDSTYSLSS | |
| | PPSREEMTKNQVSLTCLVKGFYPSDIAVE | | TLTLSKADYEKHKV | |
| | WESNGQPENNYKTTPPVLDSDGSFFLYSK | | YACEVTHQGLSSPV | |
| | LTVDKSRWQQGNVFSCSVMHEALHNHYT | | TKSFNRGEC | |
| | QKSLSLSPGK | | | |
| 34 | QVQLVQSGAEVKKPGASVKVSCKASGYT | 1660 | DIQMTQSPSSLSASV | 1748 |
| | FTDYYMHWVRQAPGQGLEWMGIINPSGG | | GDRVTITCRASQGIR | |
| | STRYAQKFQGRVTMTRDTSTSTVYMELSS | | NDLAWYQQKPGKA | |
| | LRSEDTAVYYCARVDGRRWLQSDYWGQ | | PKLLIYAASTLQNGV | |
| | GTLVTVSSSTKGPSVFPLAPSSKSTSGGTA | | PSRFSGSGSGTDFTL | |
| | ALGCLVKDYFPEPVTVSWNSGALTSGVHT | | TISSLQPEDFATYYC | |
| | FPAVLQSSGLYSLSSVVTVPSSSLGTQTYIC | | QQSYSTPWTFGQGT | |
| | NVNHKPSNTKVDKKVDKTHTCPPCPPELL | | KVEIKRTVAAPSVFI | |
| | GGPSVFLFPPKPKDTLMISRTPEVTCVVVD | | FPPSDEQLKSGTASV | |
| | VSHEDPEVKFNWYVDGVEVHNAKTKPRE | | VCLLNNFYPREAKV | |
| | EQYNSTYRVVSVLTVLHQDWLNGKEYKC | | QWKVDNALQSGNS | |
| | KVSNKALPAPIEKTISKAKQPREPQVYTLP | | QESVTEQDSKDSTYS | |
| | PSREEMTKNQVSLTCLVKGFYPSDIAVEW | | LSSTLTLSKADYEKH | |
| | ESNGQPENNYKTTPPVLDSDGSFFLYSKLT | | KVYACEVTHQGLSS | |
| | VDKSRWQQGNVFSCSVMHEALHNHYTQ | | PVTKSFNRGEC | |
| | KSLSLSPGK | | | |
| 35 | QVQLVQSGAEVKKPGASVKVSCKASGYT | 1661 | DIQMTQSPSSLSASV | 1749 |
| | FTDYYMHWVRQAPGQGLEWMGIINPSGG | | GDRVTITCRASQGIR | |
| | STRYAQKFQGRVTMTRDTSTSTVYMELSS | | NDLAWYQQKPGKA | |
| | LRSEDTAVYYCARVDGRRWLRSDYWGQ | | PKLLIYAASTLQNGV | |
| | GTLVTVSSSTKGPSVFPLAPSSKSTSGGTA | | PSRFSGSGSGTDFTL | |
| | ALGCLVKDYFPEPVTVSWNSGALTSGVHT | | TISSLQPEDFATYYC | |
| | FPAVLQSSGLYSLSSVVTVPSSSLGTQTYIC | | QQSYSTPWTFGQGT | |
| | NVNHKPSNTKVDKKVDKTHTCPPCPPELL | | KVEIKRTVAAPSVFI | |
| | GGPSVFLFPPKPKDTLMISRTPEVTCVVVD | | FPPSDEQLKSGTASV | |
| | VSHEDPEVKFNWYVDGVEVHNAKTKPRE | | VCLLNNFYPREAKV | |
| | EQYNSTYRVVSVLTVLHQDWLNGKEYKC | | QWKVDNALQSGNS | |
| | KVSNKALPAPIEKTISKAKQPREPQVYTLP | | QESVTEQDSKDSTYS | |
| | PSREEMTKNQVSLTCLVKGFYPSDIAVEW | | LSSTLTLSKADYEKH | |
| | ESNGQPENNYKTTPPVLDSDGSFFLYSKLT | | KVYACEVTHQGLSS | |
| | VDKSRWQQGNVFSCSVMHEALHNHYTQ | | PVTKSFNRGEC | |
| | KSLSLSPGK | | | |
| 36 | QVQLVQSGAEVKKPGASVKVSCKASGGT | 1662 | DIQMTQSPSSLSASV | 1750 |
| | FSSYAISWVRQAPGQGLEWLGIISPSGRSA | | GDRVTITCQASQGIN | |
| | GYGRKFQGRVTMTRDTSTSTVYMELSSLR | | NYLNWYQQKPGKA | |
| | SEDTAVYYCARTDYGGHKWYFDLWGRG | | PKLLIYAASTLQRGV | |
| | TLVTVSSSTKGPSVFPLAPSSKSTSGGTAA | | PSRFSGSGSGTDFTL | |
| | LGCLVKDYFPEPVTVSWNSGALTSGVHTF | | TISSLQPEDFATYYC | |
| | PAVLQSSGLYSLSSVVTVPSSSLGTQTYIC | | QQSYQTPLTFGGGT | |
| | NVNHKPSNTKVDKKVDKTHTCPPCPPELL | | KVEIKRTVAAPSVFI | |
| | GGPSVFLFPPKPKDTLMISRTPEVTCVVVD | | FPPSDEQLKSGTASV | |
| | VSHEDPEVKFNWYVDGVEVHNAKTKPRE | | VCLLNNFYPREAKV | |
| | EQYNSTYRVVSVLTVLHQDWLNGKEYKC | | QWKVDNALQSGNS | |
| | KVSNKALPAPIEKTISKAKQPREPQVYTLP | | QESVTEQDSKDSTYS | |
| | PSREEMTKNQVSLTCLVKGFYPSDIAVEW | | LSSTLTLSKADYEKH | |
| | ESNGQPENNYKTTPPVLDSDGSFFLYSKLT | | KVYACEVTHQGLSS | |
| | VDKSRWQQGNVFSCSVMHEALHNHYTQ | | PVTKSFNRGEC | |
| | KSLSLSPGK | | | |
| 37 | QVQLVQSGAEVKKPGASVKVSCKASGYT | 1663 | DIQMTQSPSSLSASV | 1751 |
| | FTGYYLHWVRQAPGQGLEWMGVISPSGG | | GDRVTITCRASQSISS | |
| | GTSYAQKFQGRVTMTRDTSTSTVYMELSS | | YLNWYQQKPGKAP | |
| | LRSEDTAVYYCARAGFGEGVFRHWGQGT | | KLLIYAASSLQSGVP | |
| | LVTVSSSTKGPSVFPLAPSSKSTSGGTAAL | | SRFSGSGSGTDFTLTI | |
| | GCLVKDYFPEPVTVSWNSGALTSGVHTFP | | SSLQPEDFATYYCQQ | |
| | AVLQSSGLYSLSSVVTVPSSSLGTQTYICN | | SYSTPLTFGGGTKVE | |
| | VNHKPSNTKVDKKVDKTHTCPPCPPELLG | | IKRTVAAPSVFIFPPS | |
| | GPSVFLFPPKPKDTLMISRTPEVTCVVVDV | | DEQLKSGTASVVCL | |
| | SHEDPEVKFNWYVDGVEVHNAKTKPREE | | LNNFYPREAKVQWK | |
| | QYNSTYRVVSVLTVLHQDWLNGKEYKCK | | VDNALQSGNSQESV | |
| | VSNKALPAPIEKTISKAKQPREPQVYTLPPS | | TEQDSKDSTYSLSST | |
| | REEMTKNQVSLTCLVKGFYPSDIAVEWES | | LTLSKADYEKHKVY | |
| | NGQPENNYKTTPPVLDSDGSFFLYSKLTV | | ACEVTHQGLSSPVT | |
| | DKSRWQQGNVFSCSVMHEALHNHYTQKS | | KSFNRGEC | |
| | LSLSPGK | | | |
| 38 | QVQLVQSGAEVKKPGASVKVSCKASGYS | 1664 | DIQMTQSPSSLSASV | 1752 |
| | FTSHAISWVRQAPGQGLEWMGWIKPNSG | | GDRVTITCRASQGIS | |
| | DTKYAQKFQGRVTMTRDTSTSTVYMELSS | | NYLAWYQQKPGKA | |
| | LRSEDTAVYYCARGSDDYYGSYYFDYWG | | PKLLIYTASTLQSGV | |
| | QGTLVTVSSSTKGPSVFPLAPSSKSTSGGT | | PSRFSGSGSGTDFTL | |
| | AALGCLVKDYFPEPVTVSWNSGALTSGVH | | TISSLQPEDFATYYC | |
| | TFPAVLQSSGLYSLSSVVTVPSSSLGTQTYI | | QQSYSTPLTFGGGTK | |
| | CNVNHKPSNTKVDKKVDKTHTCPPCPPEL | | VEIKRTVAAPSVFIFP | |
| | LGGPSVFLFPPKPKDTLMISRTPEVTCVVV | | PSDEQLKSGTASVVC | |
| | DVSHEDPEVKFNWYVDGVEVHNAKTKPR | | LLNNFYPREAKVQW | |
| | EEQYNSTYRVVSVLTVLHQDWLNGKEYK | | KVDNALQSGNSQES | |
| | CKVSNKALPAPIEKTISKAKQPREPQVYTL | | VTEQDSKDSTYSLSS | |
| | PPSREEMTKNQVSLTCLVKGFYPSDIAVE | | TLTLSKADYEKHKV | |
| | WESNGQPENNYKTTPPVLDSDGSFFLYSK | | YACEVTHQGLSSPV | |
| | LTVDKSRWQQGNVFSCSVMHEALHNHYT | | TKSFNRGEC | |
| | QKSLSLSPGK | | | |
| 39 | EVQLLESGGGLVQPGGSLRLSCAASGFTFR | 1665 | DIQMTQSPSSLSASV | 1753 |
| | NYGMGWVRQAPGKGLEWVSAISGSGGST | | GDRVTITCRASQGIS | |
| | YYADSVKGRFTISRDNSKNTLYLQMNSLR | | NDLAWYQQKPGKA | |
| | AEDTAVYYCARVKFYGMDVWGQGTTVT | | PKLLIYGASNLETGV | |
| | VSSSTKGPSVFPLAPSSKSTSGGTAALGCL | | PSRFSGSGSGTDFTL | |
| | VKDYFPEPVTVSWNSGALTSGVHTFPAVL | | TISSLQPEDFATYYC | |
| | QSSGLYSLSSVVTVPSSSLGTQTYICNVNH | | QQANSFPFTFGPGTK | |
| | KPSNTKVDKKVDKTHTCPPCPPELLGGPS | | VDIKRTVAAPSVFIFP | |
| | VFLFPPKPKDTLMISRTPEVTCVVVDVSHE | | PSDEQLKSGTASVVC | |
| | DPEVKFNWYVDGVEVHNAKTKPREEQYN | | LLNNFYPREAKVQW | |
| | STYRVVSVLTVLHQDWLNGKEYKCKVSN | | KVDNALQSGNSQES | |
| | KALPAPIEKTISKAKQPREPQVYTLPPSREE | | VTEQDSKDSTYSLSS | |
| | MTKNQVSLTCLVKGFYPSDIAVEWESNGQ | | TLTLSKADYEKHKV | |
| | PENNYKTTPPVLDSDGSFFLYSKLTVDKSR | | YACEVTHQGLSSPV | |
| | WQQGNVFSCSVMHEALHNHYTQKSLSLS | | TKSFNRGEC | |
| | PGK | | | |
| 40 | QVQLVQSGAEVKKPGASVKVSCKASGYT | 1666 | DIQMTQSPSSLSASV | 1754 |
| | FTDYHMHWVRQAPGQGLEWMGWMSPNS | | GDRVTITCRVSQGIS | |
| | GNTGYAQNFQGRVTMTRDTSTSTVYMEL | | SYLNWYQQKPGKAP | |
| | SSLRSEDTAVYYCARADYYGSDYVKFDY | | KLLIYEASTLESGVP | |
| | WGQGTLVTVSSSTKGPSVFPLAPSSKSTSG | | SRFSGSGSGTDFTLTI | |
| | GTAALGCLVKDYFPEPVTVSWNSGALTSG | | SSLQPEDFATYYCQQ | |
| | VHTFPAVLQSSGLYSLSSVVTVPSSSLGTQ | | GYSTPPTFGQGTKVE | |
| | TYICNVNHKPSNTKVDKKVDKTHTCPPCP | | IKRTVAAPSVFIFPPS | |
| | PELLGGPSVFLFPPKPKDTLMISRTPEVTCV | | DEQLKSGTASVVCL | |
| | VVDVSHEDPEVKFNWYVDGVEVHNAKTK | | LNNFYPREAKVQWK | |
| | PREEQYNSTYRVVSVLTVLHQDWLNGKE | | VDNALQSGNSQESV | |
| | YKCKVSNKALPAPIEKTISKAKQPREPQVY | | TEQDSKDSTYSLSST | |
| | TLPPSREEMTKNQVSLTCLVKGFYPSDIAV | | LTLSKADYEKHKVY | |
| | EWESNGQPENNYKTTPPVLDSDGSFFLYS | | ACEVTHQGLSSPVT | |
| | KLTVDKSRWQQGNVFSCSVMHEALHNHY | | KSFNRGEC | |
| | TQKSLSLSPGK | | | |
| 41 | QVQLVQSGAEVKKPGASVKVSCKASGYT | 1667 | DIVMTQSPLSLPVTP | 1755 |
| | FPNYGISWVRQAPGQGLEWMGWINPNSG | | GEPASISCRSSQSLLQ | |
| | GTKYAQRFQGRVTMTRDTSTSTVYMELSS | | SNGYNYLDWYLQKP | |
| | LRSEDTAVYYCARDRDILTGYYHFDYWG | | GQSPQLLIYLGSNRA | |
| | QGTLVTVSSSTKGPSVFPLAPSSKSTSGGT | | SGVPDRFSGSGSGTD | |
| | AALGCLVKDYFPEPVTVSWNSGALTSGVH | | FTLKISRVEAEDVGV | |
| | TFPAVLQSSGLYSLSSVVTVPSSSLGTQTYI | | YYCMQSTHWPLTFG | |
| | CNVNHKPSNTKVDKKVDKTHTCPPCPPEL | | QGTRLEIKRTVAAPS | |
| | LGGPSVFLFPPKPKDTLMISRTPEVTCVVV | | VFIFPPSDEQLKSGT | |
| | DVSHEDPEVKFNWYVDGVEVHNAKTKPR | | ASVVCLLNNFYPRE | |
| | EEQYNSTYRVVSVLTVLHQDWLNGKEYK | | AKVQWKVDNALQS | |
| | CKVSNKALPAPIEKTISKAKQPREPQVYTL | | GNSQESVTEQDSKD | |
| | PPSREEMTKNQVSLTCLVKGFYPSDIAVE | | STYSLSSTLTLSKAD | |
| | WESNGQPENNYKTTPPVLDSDGSFFLYSK | | YEKHKVYACEVTHQ | |
| | LTVDKSRWQQGNVFSCSVMHEALHNHYT | | GLSSPVTKSFNRGEC | |
| | QKSLSLSPGK | | | |
| 42 | QVQLVQSGAEVKKPGASVKVSCKASGYT | 1668 | DIQMTQSPSSLSASV | 1756 |
| | FTDYFMHWVRQAPGQGLEWMGWINPNS | | GDRVTITCRASQGIS | |
| | GNTGYAQKFQGRVTMTRDTSTSTVYMEL | | NNLNWYQQKPGKA | |
| | SSLRSEDTAVYYCARLNDYGDYGGPATLD | | PKLLIYAASSLQSGV | |
| | YWGQGTLVTVSSSTKGPSVFPLAPSSKSTS | | PSRFSGSGSGTDFTL | |
| | GGTAALGCLVKDYFPEPVTVSWNSGALTS | | TISSLQPEDFATYYC | |
| | GVHTFPAVLQSSGLYSLSSVVTVPSSSLGT | | QQSYSTPPTFGQGTK | |
| | QTYICNVNHKPSNTKVDKKVDKTHTCPPC | | LEIKRTVAAPSVFIFP | |
| | PPELLGGPSVFLFPPKPKDTLMISRTPEVTC | | PSDEQLKSGTASVVC | |
| | VVVDVSHEDPEVKFNWYVDGVEVHNAKT | | LLNNFYPREAKVQW | |
| | KPREEQYNSTYRVVSVLTVLHQDWLNGK | | KVDNALQSGNSQES | |
| | EYKCKVSNKALPAPIEKTISKAKQPREPQV | | VTEQDSKDSTYSLSS | |
| | YTLPPSREEMTKNQVSLTCLVKGFYPSDIA | | TLTLSKADYEKHKV | |
| | VEWESNGQPENNYKTTPPVLDSDGSFFLY | | YACEVTHQGLSSPV | |
| | SKLTVDKSRWQQGNVFSCSVMHEALHNH | | TKSFNRGEC | |
| | YTQKSLSLSPGK | | | |
| 43 | QVQLVQSGAEVKKPGASVKVSCKASGYT | 1669 | DIQMTQSPSSLSASV | 1757 |
| | FTNYYMHWVRQAPGQGLEWLGWISPYSG | | GDRVTITCRASQSIST | |
| | DTKYAQTLQGRVTMTRDTSTSTVYMELSS | | YLNWYQQKPGKAP | |
| | LRSEDTAVYYCARESMDRLDYWGQGTLV | | KLLIYDASNLETGVP | |
| | TVSSSTKGPSVFPLAPSSKSTSGGTAALGC | | SRFSGSGSGTDFTLTI | |
| | LVKDYFPEPVTVSWNSGALTSGVHTFPAV | | SSLQPEDFATYYCQQ | |
| | LQSSGLYSLSSVVTVPSSSLGTQTYICNVN | | SYSTPVLTFGGGTKV | |
| | HKPSNTKVDKKVDKTHTCPPCPPELLGGP | | EIKRTVAAPSVFIFPP | |
| | SVFLFPPKPKDTLMISRTPEVTCVVVDVSH | | SDEQLKSGTASVVC | |
| | EDPEVKFNWYVDGVEVHNAKTKPREEQY | | LLNNFYPREAKVQW | |
| | NSTYRVVSVLTVLHQDWLNGKEYKCKVS | | KVDNALQSGNSQES | |
| | NKALPAPIEKTISKAKQPREPQVYTLPPSRE | | VTEQDSKDSTYSLSS | |
| | EMTKNQVSLTCLVKGFYPSDIAVEWESNG | | TLTLSKADYEKHKV | |
| | QPENNYKTTPPVLDSDGSFFLYSKLTVDKS | | YACEVTHQGLSSPV | |
| | RWQQGNVFSCSVMHEALHNHYTQKSLSL | | TKSFNRGEC | |
| | SPGK | | | |
| 44 | EVQLLESGGGLVQPGGSLRLSCAASGFTFS | 1670 | DIVMTQSPLSLPVTP | 1758 |
| | SYAMHWVRQAPGKGLEWVADISGSGGLT | | GEPASISCRSSQSLLH | |
| | YYADSVKGRFTISRDNSKNTLYLQMNSLR | | SNGYNYLDWYLQKP | |
| | AEDTAVYYCAREGDQYSSSSFFDYWGQG | | GQSPQLLIYLGSNRA | |
| | TLVTVSSSTKGPSVFPLAPSSKSTSGGTAA | | SGVPDRFSGSGSGTD | |
| | LGCLVKDYFPEPVTVSWNSGALTSGVHTF | | FTLKISRVEAEDVGV | |
| | PAVLQSSGLYSLSSVVTVPSSSLGTQTYIC | | YYCMQALQPPPTFG | |
| | NVNHKPSNTKVDKKVDKTHTCPPCPPELL | | QGTRLEIKRTVAAPS | |
| | GGPSVFLFPPKPKDTLMISRTPEVTCVVVD | | VFIFPPSDEQLKSGT | |
| | VSHEDPEVKFNWYVDGVEVHNAKTKPRE | | ASVVCLLNNFYPRE | |
| | EQYNSTYRVVSVLTVLHQDWLNGKEYKC | | AKVQWKVDNALQS | |
| | KVSNKALPAPIEKTISKAKQPREPQVYTLP | | GNSQESVTEQDSKD | |
| | PSREEMTKNQVSLTCLVKGFYPSDIAVEW | | STYSLSSTLTLSKAD | |
| | ESNGQPENNYKTTPPVLDSDGSFFLYSKLT | | YEKHKVYACEVTHQ | |
| | VDKSRWQQGNVFSCSVMHEALHNHYTQ | | GLSSPVTKSFNRGEC | |
| | KSLSLSPGK | | | |
| 45 | EVQLVESGGGLVKPGGSLRLSCAASGFTF | 1671 | DIQMTQSPSSLSASV | 1759 |
| | DEFGMNWVRQAPGKGLEWISYISGDSGYT | | GDRVTITCQASQDID | |
| | NCADSVKGRFTISRDDSKNTLYLQMNSLK | | IYLNWYQQKPGKAP | |
| | TEDTAVYYCAAGYGGYYFDYWGQGTLV | | KLLIYAASTLESGVP | |
| | TVSSSTKGPSVFPLAPSSKSTSGGTAALGC | | SRFSGSGSGTDFTLTI | |
| | LVKDYFPEPVTVSWNSGALTSGVHTFPAV | | SSLQPEDFATYYCQQ | |
| | LQSSGLYSLSSVVTVPSSSLGTQTYICNVN | | SYSTPPTFGGGTKVE | |
| | HKPSNTKVDKKVDKTHTCPPCPPELLGGP | | IKRTVAAPSVFIFPPS | |
| | SVFLFPPKPKDTLMISRTPEVTCVVVDVSH | | DEQLKSGTASVVCL | |
| | EDPEVKFNWYVDGVEVHNAKTKPREEQY | | LNNFYPREAKVQWK | |
| | NSTYRVVSVLTVLHQDWLNGKEYKCKVS | | VDNALQSGNSQESV | |
| | NKALPAPIEKTISKAKQPREPQVYTLPPSRE | | TEQDSKDSTYSLSST | |
| | EMTKNQVSLTCLVKGFYPSDIAVEWESNG | | LTLSKADYEKHKVY | |
| | QPENNYKTTPPVLDSDGSFFLYSKLTVDKS | | ACEVTHQGLSSPVT | |
| | RWQQGNVFSCSVMHEALHNHYTQKSLSL | | KSFNRGEC | |
| | SPGK | | | |
| 46 | QVQLVQSGAEVKKPGASVKVSCKASGYT | 1672 | DIQMTQSPSSLSASV | 1760 |
| | FTSYYMHWVRQAPGQGLEWMGMINPSA | | GDRVTITCRASQSIST | |
| | GSTSYAQKFQGRVTMTRDTSTSTVYMELS | | YLNWYQQKPGKAP | |
| | SLRSEDTAVYYCASVDSSGWYAPFDYWG | | KLLIYDASNLETGVP | |
| | QGTLVTVSSSTKGPSVFPLAPSSKSTSGGT | | SRFSGSGSGTDFTLTI | |
| | AALGCLVKDYFPEPVTVSWNSGALTSGVH | | SSLQPEDFATYYCQQ | |
| | TFPAVLQSSGLYSLSSVVTVPSSSLGTQTYI | | ANSFPPTFGGGTKVE | |
| | CNVNHKPSNTKVDKKVDKTHTCPPCPPEL | | IKRTVAAPSVFIFPPS | |
| | LGGPSVFLFPPKPKDTLMISRTPEVTCVVV | | DEQLKSGTASVVCL | |
| | DVSHEDPEVKFNWYVDGVEVHNAKTKPR | | LNNFYPREAKVQWK | |
| | EEQYNSTYRVVSVLTVLHQDWLNGKEYK | | VDNALQSGNSQESV | |
| | CKVSNKALPAPIEKTISKAKQPREPQVYTL | | TEQDSKDSTYSLSST | |
| | PPSREEMTKNQVSLTCLVKGFYPSDIAVE | | LTLSKADYEKHKVY | |
| | WESNGQPENNYKTTPPVLDSDGSFFLYSK | | ACEVTHQGLSSPVT | |
| | LTVDKSRWQQGNVFSCSVMHEALHNHYT | | KSFNRGEC | |
| | QKSLSLSPGK | | | |
| 47 | EVQLLESGGGLVQPGGSLRLSCAASGFTF | 1673 | DIVMTQSPLSLPVTP | 1761 |
| | DEYAMHWVRQAPGKGLEWVSAIGAGGST | | GEPASISCRSSQSLLH | |
| | YYADSVKGRFTISRDNSKNTLYLQMNSLR | | SNGYNYLDWYLQKP | |
| | AEDTAVYYCASSLGPELRGVDYYYYGMD | | GQSPQLLIYAASSLQ | |
| | VWGQGTTVTVSSSTKGPSVFPLAPSSKSTS | | SGVPDRFSGSGSGTD | |
| | GGTAALGCLVKDYFPEPVTVSWNSGALTS | | FTLKISRVEAEDVGV | |
| | GVHTFPAVLQSSGLYSLSSVVTVPSSSLGT | | YYCMQGIQWPWTF | |
| | QTYICNVNHKPSNTKVDKKVDKTHTCPPC | | GQGTKVEIKRTVAA | |
| | PPELLGGPSVFLFPPKPKDTLMISRTPEVTC | | PSVFIFPPSDEQLKSG | |
| | VVVDVSHEDPEVKFNWYVDGVEVHNAKT | | TASVVCLLNNFYPRE | |
| | KPREEQYNSTYRVVSVLTVLHQDWLNGK | | AKVQWKVDNALQS | |
| | EYKCKVSNKALPAPIEKTISKAKQPREPQV | | GNSQESVTEQDSKD | |
| | YTLPPSREEMTKNQVSLTCLVKGFYPSDIA | | STYSLSSTLTLSKAD | |
| | VEWESNGQPENNYKTTPPVLDSDGSFFLY | | YEKHKVYACEVTHQ | |
| | SKLTVDKSRWQQGNVFSCSVMHEALHNH | | GLSSPVTKSFNRGEC | |
| | YTQKSLSLSPGK | | | |
| 48 | EVQLLESGGGLVQPGGSLRLSCAASGFNF | 1674 | DIQMTQSPSSLSASV | 1762 |
| | DDYAMHWVRQAPGKGLEWVSVIYSGGST | | GDRVTITCRASQSIST | |
| | YYADSVKGRFTISRDNSKNTLYLQMNSLR | | YVNWYQQKPGKAP | |
| | AEDTAVYYCTRHDFDYWGQGTLVTVSSS | | KLLIYAASSLQSGVP | |
| | TKGPSVFPLAPSSKSTSGGTAALGCLVKDY | | SRFSGSGSGTDFTLTI | |
| | FPEPVTVSWNSGALTSGVHTFPAVLQSSGL | | SSLQPEDFATYYCQQ | |
| | YSLSSVVTVPSSSLGTQTYICNVNHKPSNT | | DYSYPYTFGQGTKV | |
| | KVDKKVDKTHTCPPCPPELLGGPSVFLFPP | | EIKRTVAAPSVFIFPP | |
| | KPKDTLMISRTPEVTCVVVDVSHEDPEVK | | SDEQLKSGTASVVC | |
| | FNWYVDGVEVHNAKTKPREEQYNSTYRV | | LLNNFYPREAKVQW | |
| | VSVLTVLHQDWLNGKEYKCKVSNKALPA | | KVDNALQSGNSQES | |
| | PIEKTISKAKQPREPQVYTLPPSREEMTKN | | VTEQDSKDSTYSLSS | |
| | QVSLTCLVKGFYPSDIAVEWESNGQPENN | | TLTLSKADYEKHKV | |
| | YKTTPPVLDSDGSFFLYSKLTVDKSRWQQ | | YACEVTHQGLSSPV | |
| | GNVFSCSVMHEALHNHYTQKSLSLSPGK | | TKSFNRGEC | |
| 49 | EVQLVESGGGLVKPGGSLRLSCAASGFTFS | 1675 | DIQMTQSPSSLSASV | 1763 |
| | DYALHWVRQAPGKGLEWVSLISGDGGST | | GDRVTITCRASQSIST | |
| | YYADSVKGRFTISRDDSKNTLYLQMNSLK | | WLAWYQQKPGKAP | |
| | TEDTAVYYCARDLGGERSYWGQGTLVTV | | KLLIYAASTLQSGVP | |
| | SSSTKGPSVFPLAPSSKSTSGGTAALGCLV | | SRFSGSGSGTDFTLTI | |
| | KDYFPEPVTVSWNSGALTSGVHTFPAVLQ | | SSLQPEDFATYYCLQ | |
| | SSGLYSLSSVVTVPSSSLGTQTYICNVNHK | | DYSYPPTFGQGTKV | |
| | PSNTKVDKKVDKTHTCPPCPPELLGGPSVF | | EIKRTVAAPSVFIFPP | |
| | LFPPKPKDTLMISRTPEVTCVVVDVSHEDP | | SDEQLKSGTASVVC | |
| | EVKFNWYVDGVEVHNAKTKPREEQYNST | | LLNNFYPREAKVQW | |
| | YRVVSVLTVLHQDWLNGKEYKCKVSNKA | | KVDNALQSGNSQES | |
| | LPAPIEKTISKAKQPREPQVYTLPPSREEMT | | VTEQDSKDSTYSLSS | |
| | KNQVSLTCLVKGFYPSDIAVEWESNGQPE | | TLTLSKADYEKHKV | |
| | NNYKTTPPVLDSDGSFFLYSKLTVDKSRW | | YACEVTHQGLSSPV | |
| | QQGNVFSCSVMHEALHNHYTQKSLSLSPG | | TKSFNRGEC | |
| | K | | | |
| 50 | QVQLVQSGAEVKKPGASVKVSCKASGYT | 1676 | DIQMTQSPSSLSASV | 1764 |
| | FTDYYMHWVRQAPGQGLEWMGIINPSDG | | GDRVTITCRASQSISS | |
| | STTYAQSFQGRVTMTRDTSTSTVYMELSS | | WLAWYQQKPGKAP | |
| | LRSEDTAVYYCARDELPDSSGWYGYFQH | | KLLIYAASSLQSGVP | |
| | WGQGTLVTVSSSTKGPSVFPLAPSSKSTSG | | SRFSGSGSGTDFTLTI | |
| | GTAALGCLVKDYFPEPVTVSWNSGALTSG | | SSLQPEDFATYYCQQ | |
| | VHTFPAVLQSSGLYSLSSVVTVPSSSLGTQ | | SYDIPLTFGGGTKVE | |
| | TYICNVNHKPSNTKVDKKVDKTHTCPPCP | | IKRTVAAPSVFIFPPS | |
| | PELLGGPSVFLFPPKPKDTLMISRTPEVTCV | | DEQLKSGTASVVCL | |
| | VVDVSHEDPEVKFNWYVDGVEVHNAKTK | | LNNFYPREAKVQWK | |
| | PREEQYNSTYRVVSVLTVLHQDWLNGKE | | VDNALQSGNSQESV | |
| | YKCKVSNKALPAPIEKTISKAKQPREPQVY | | TEQDSKDSTYSLSST | |
| | TLPPSREEMTKNQVSLTCLVKGFYPSDIAV | | LTLSKADYEKHKVY | |
| | EWESNGQPENNYKTTPPVLDSDGSFFLYS | | ACEVTHQGLSSPVT | |
| | KLTVDKSRWQQGNVFSCSVMHEALHNHY | | KSFNRGEC | |
| | TQKSLSLSPGK | | | |
| 51 | QVQLVQSGAEVKKPGSSVKVSCKASGGTF | 1677 | DIQMTQSPSSLSASV | 1765 |
| | SSYAISWVRQAPGQGLEWMGEIIPFFGTAN | | GDRVTITCQASQDIS | |
| | YAQKFQGRVTITADESTSTAYMELSSLRSE | | NLLNWYQQKPGKAP | |
| | DTAVYYCARAEYGGDLDYWGQGTLVTVS | | KLLIYAASTLQSGVP | |
| | SSTKGPSVFPLAPSSKSTSGGTAALGCLVK | | SRFSGSGSGTDFTLTI | |
| | DYFPEPVTVSWNSGALTSGVHTFPAVLQS | | SSLQPEDFATYYCQQ | |
| | SGLYSLSSVVTVPSSSLGTQTYICNVNHKP | | SYNTPWTFGPGTKV | |
| | SNTKVDKKVDKTHTCPPCPPELLGGPSVFL | | DIKRTVAAPSVFIFPP | |
| | FPPKPKDTLMISRTPEVTCVVVDVSHEDPE | | SDEQLKSGTASVVC | |
| | VKFNWYVDGVEVHNAKTKPREEQYNSTY | | LLNNFYPREAKVQW | |
| | RVVSVLTVLHQDWLNGKEYKCKVSNKAL | | KVDNALQSGNSQES | |
| | PAPIEKTISKAKQPREPQVYTLPPSREEMTK | | VTEQDSKDSTYSLSS | |
| | NQVSLTCLVKGFYPSDIAVEWESNGQPEN | | TLTLSKADYEKHKV | |
| | NYKTTPPVLDSDGSFFLYSKLTVDKSRWQ | | YACEVTHQGLSSPV | |
| | QGNVFSCSVMHEALHNHYTQKSLSLSPGK | | TKSFNRGEC | |
| 52 | QVQLVQSGAEVKKPGASVKVSCKASGDT | 1678 | DIQMTQSPSSLSASV | 1766 |
| | FTRHYVHWVRQAPGQGLEWMGIINPRGG | | GDRVTITCQASQDIH | |
| | THYAQKFQGRVTMTRDTSTSTVYMELSSL | | NYLNWYQQKPGKA | |
| | RSEDTAVYYCARRDCSGGSCYSDLDYWG | | PKLLIYQASSLESGV | |
| | QGTLVTVSSSTKGPSVFPLAPSSKSTSGGT | | PSRFSGSGSGTDFTL | |
| | AALGCLVKDYFPEPVTVSWNSGALTSGVH | | TISSLQPEDFATYYC | |
| | TFPAVLQSSGLYSLSSVVTVPSSSLGTQTYI | | QQANSFPLTFGGGT | |
| | CNVNHKPSNTKVDKKVDKTHTCPPCPPEL | | KLEIKRTVAAPSVFIF | |
| | LGGPSVFLFPPKPKDTLMISRTPEVTCVVV | | PPSDEQLKSGTASVV | |
| | DVSHEDPEVKFNWYVDGVEVHNAKTKPR | | CLLNNFYPREAKVQ | |
| | EEQYNSTYRVVSVLTVLHQDWLNGKEYK | | WKVDNALQSGNSQE | |
| | CKVSNKALPAPIEKTISKAKQPREPQVYTL | | SVTEQDSKDSTYSLS | |
| | PPSREEMTKNQVSLTCLVKGFYPSDIAVE | | STLTLSKADYEKHK | |
| | WESNGQPENNYKTTPPVLDSDGSFFLYSK | | VYACEVTHQGLSSP | |
| | LTVDKSRWQQGNVFSCSVMHEALHNHYT | | VTKSFNRGEC | |
| | QKSLSLSPGK | | | |
| 53 | QVQLVQSGAEVKKPGASVKVSCKASGGT | 1679 | DIQMTQSPSSLSASV | 1767 |
| | FSSYAISWVRQAPGQGLEWMGWINPDSG | | GDRVTITCRASQNIG | |
| | DASYARKFQGRVTMTRDTSTSTVYMELSS | | SWLAWYQQKPGKA | |
| | LRSEDTAVYYCATFGEEAFDIWGQGTMVT | | PKLLIYGASILQSGVP | |
| | VSSSTKGPSVFPLAPSSKSTSGGTAALGCL | | SRFSGSGSGTDFTLTI | |
| | VKDYFPEPVTVSWNSGALTSGVHTFPAVL | | SSLQPEDFATYYCQQ | |
| | QSSGLYSLSSVVTVPSSSLGTQTYICNVNH | | ANSFPLTFGGGTKLE | |
| | KPSNTKVDKKVDKTHTCPPCPPELLGGPS | | IKRTVAAPSVFIFPPS | |
| | VFLFPPKPKDTLMISRTPEVTCVVVDVSHE | | DEQLKSGTASVVCL | |
| | DPEVKFNWYVDGVEVHNAKTKPREEQYN | | LNNFYPREAKVQWK | |
| | STYRVVSVLTVLHQDWLNGKEYKCKVSN | | VDNALQSGNSQESV | |
| | KALPAPIEKTISKAKQPREPQVYTLPPSREE | | TEQDSKDSTYSLSST | |
| | MTKNQVSLTCLVKGFYPSDIAVEWESNGQ | | LTLSKADYEKHKVY | |
| | PENNYKTTPPVLDSDGSFFLYSKLTVDKSR | | ACEVTHQGLSSPVT | |
| | WQQGNVFSCSVMHEALHNHYTQKSLSLS | | KSFNRGEC | |
| | PGK | | | |
| 54 | QVQLVQSGAEVKKPGASVKVSCKASGGT | 1680 | DIQMTQSPSSLSASV | 1768 |
| | FSSYAISWVRQAPGQGLEWMGWIDPKNG | | GDRVTITCRASQGIG | |
| | DTNYAQKFQGRVTMTRDTSTSTVYMELSS | | NWLAWYQQKPGKA | |
| | LRSEDTAVYYCATEGSHHPYYYYGMDVW | | PKLLIYEASTLQSGV | |
| | GQGTTVTVSSSTKGPSVFPLAPSSKSTSGG | | PSRFSGSGSGTDFTL | |
| | TAALGCLVKDYFPEPVTVSWNSGALTSGV | | TISSLQPEDFATYYC | |
| | HTFPAVLQSSGLYSLSSVVTVPSSSLGTQT | | HQYNAYPWTFGQGT | |
| | YICNVNHKPSNTKVDKKVDKTHTCPPCPP | | KVEIKRTVAAPSVFI | |
| | ELLGGPSVFLFPPKPKDTLMISRTPEVTCV | | FPPSDEQLKSGTASV | |
| | VVDVSHEDPEVKFNWYVDGVEVHNAKTK | | VCLLNNFYPREAKV | |
| | PREEQYNSTYRVVSVLTVLHQDWLNGKE | | QWKVDNALQSGNS | |
| | YKCKVSNKALPAPIEKTISKAKQPREPQVY | | QESVTEQDSKDSTYS | |
| | TLPPSREEMTKNQVSLTCLVKGFYPSDIAV | | LSSTLTLSKADYEKH | |
| | EWESNGQPENNYKTTPPVLDSDGSFFLYS | | KVYACEVTHQGLSS | |
| | KLTVDKSRWQQGNVFSCSVMHEALHNHY | | PVTKSFNRGEC | |
| | TQKSLSLSPGK | | | |
| 55 | QVQLVQSGAEVKKPGASVKVSCKASGYT | 1681 | DIQMTQSPSSLSASV | 1769 |
| | FTGYHMHWVRQAPGQGLEWMGWINPNT | | GDRVTITCQASQDIS | |
| | GGTNYAQKFQGRVTMTRDTSTSTVYMEL | | NYLNWYQQKPGKA | |
| | SSLRSEDTAVYYCARPNTAMVPPYYYYY | | PKLLIYAASSLQSGV | |
| | GMDVWGQGTLVTVSSSTKGPSVFPLAPSS | | PSRFSGSGSGTDFTL | |
| | KSTSGGTAALGCLVKDYFPEPVTVSWNSG | | TISSLQPEDFATYYC | |
| | ALTSGVHTFPAVLQSSGLYSLSSVVTVPSS | | QQYNSYPLTFGQGT | |
| | SLGTQTYICNVNHKPSNTKVDKKVDKTHT | | KLEIKRTVAAPSVFIF | |
| | CPPCPPELLGGPSVFLFPPKPKDTLMISRTP | | PPSDEQLKSGTASVV | |
| | EVTCVVVDVSHEDPEVKFNWYVDGVEVH | | CLLNNFYPREAKVQ | |
| | NAKTKPREEQYNSTYRVVSVLTVLHQDW | | WKVDNALQSGNSQE | |
| | LNGKEYKCKVSNKALPAPIEKTISKAKQPR | | SVTEQDSKDSTYSLS | |
| | EPQVYTLPPSREEMTKNQVSLTCLVKGFY | | STLTLSKADYEKHK | |
| | PSDIAVEWESNGQPENNYKTTPPVLDSDG | | VYACEVTHQGLSSP | |
| | SFFLYSKLTVDKSRWQQGNVFSCSVMHEA | | VTKSFNRGEC | |
| | LHNHYTQKSLSLSPGK | | | |
| 56 | QVQLVQSGAEVKKPGASVKVSCKASGYT | 1682 | DIQMTQSPSSLSASV | 1770 |
| | FTSYDINWVRQAPGQGLEWMGWMNPNS | | GDRVTITCRASHSISS | |
| | GNTGYAQKFQGRVTMTRDTSTSTVYMEL | | WLAWYQQKPGKAP | |
| | SSLRSEDTAVYYCARVSATGTYGLDYWG | | KLLIYDASNLETGVP | |
| | QGTLVTVSSSTKGPSVFPLAPSSKSTSGGT | | SRFSGSGSGTDFTLTI | |
| | AALGCLVKDYFPEPVTVSWNSGALTSGVH | | SSLQPEDFATYYCQQ | |
| | TFPAVLQSSGLYSLSSVVTVPSSSLGTQTYI | | ADSFPLTFGGGTKVE | |
| | CNVNHKPSNTKVDKKVDKTHTCPPCPPEL | | IKRTVAAPSVFIFPPS | |
| | LGGPSVFLFPPKPKDTLMISRTPEVTCVVV | | DEQLKSGTASVVCL | |
| | DVSHEDPEVKFNWYVDGVEVHNAKTKPR | | LNNFYPREAKVQWK | |
| | EEQYNSTYRVVSVLTVLHQDWLNGKEYK | | VDNALQSGNSQESV | |
| | CKVSNKALPAPIEKTISKAKQPREPQVYTL | | TEQDSKDSTYSLSST | |
| | PPSREEMTKNQVSLTCLVKGFYPSDIAVE | | LTLSKADYEKHKVY | |
| | WESNGQPENNYKTTPPVLDSDGSFFLYSK | | ACEVTHQGLSSPVT | |
| | LTVDKSRWQQGNVFSCSVMHEALHNHYT | | KSFNRGEC | |
| | QKSLSLSPGK | | | |
| 57 | QVQLVQSGAEVKKPGASVKVSCKASGYT | 1683 | DIQMTQSPSSLSASV | 1771 |
| | FNNYGITWVRQAPGQGLEWMGIINPITGV | | GDRVTITCQASQDIN | |
| | TTYAQNFQGRVTMTRDTSTSTVYMELSSL | | DYLNWYQQKPGKA | |
| | RSEDTAVYYCASGEQQLVLFDYWGQGTL | | PKLLIYGASNLQSGV | |
| | VTVSSSTKGPSVFPLAPSSKSTSGGTAALG | | PSRFSGSGSGTDFTL | |
| | CLVKDYFPEPVTVSWNSGALTSGVHTFPA | | TISSLQPEDFATYYC | |
| | VLQSSGLYSLSSVVTVPSSSLGTQTYICNV | | LQHNSYPLTFGQGT | |
| | NHKPSNTKVDKKVDKTHTCPPCPPELLGG | | KLEIKRTVAAPSVFIF | |
| | PSVFLFPPKPKDTLMISRTPEVTCVVVDVS | | PPSDEQLKSGTASVV | |
| | HEDPEVKFNWYVDGVEVHNAKTKPREEQ | | CLLNNFYPREAKVQ | |
| | YNSTYRVVSVLTVLHQDWLNGKEYKCKV | | WKVDNALQSGNSQE | |
| | SNKALPAPIEKTISKAKQPREPQVYTLPPSR | | SVTEQDSKDSTYSLS | |
| | EEMTKNQVSLTCLVKGFYPSDIAVEWESN | | STLTLSKADYEKHK | |
| | GQPENNYKTTPPVLDSDGSFFLYSKLTVD | | VYACEVTHQGLSSP | |
| | KSRWQQGNVFSCSVMHEALHNHYTQKSL | | VTKSFNRGEC | |
| | SLSPGK | | | |
| 58 | QVQLVQSGAEVKKPGASVKVSCKASGYT | 1684 | DIQMTQSPSSLSASV | 1772 |
| | FTDYYLHWVRQAPGQGLEWMGWMNPNS | | GDRVTITCRASQGIS | |
| | GNTGYAQKFQGRVTMTRDTSTSTVYMEL | | NYLAWYQQKPGKA | |
| | SSLRSEDTAVYYCAADVITAYGMDVWGQ | | PKLLIYDASNLETGV | |
| | GTMVTVSSSTKGPSVFPLAPSSKSTSGGTA | | PSRFSGSGSGTDFTL | |
| | ALGCLVKDYFPEPVTVSWNSGALTSGVHT | | TISSLQPEDFATYYC | |
| | FPAVLQSSGLYSLSSVVTVPSSSLGTQTYIC | | QQSYNVPPTFGQGT | |
| | NVNHKPSNTKVDKKVDKTHTCPPCPPELL | | KVEIKRTVAAPSVFI | |
| | GGPSVFLFPPKPKDTLMISRTPEVTCVVVD | | FPPSDEQLKSGTASV | |
| | VSHEDPEVKFNWYVDGVEVHNAKTKPRE | | VCLLNNFYPREAKV | |
| | EQYNSTYRVVSVLTVLHQDWLNGKEYKC | | QWKVDNALQSGNS | |
| | KVSNKALPAPIEKTISKAKQPREPQVYTLP | | QESVTEQDSKDSTYS | |
| | PSREEMTKNQVSLTCLVKGFYPSDIAVEW | | LSSTLTLSKADYEKH | |
| | ESNGQPENNYKTTPPVLDSDGSFFLYSKLT | | KVYACEVTHQGLSS | |
| | VDKSRWQQGNVFSCSVMHEALHNHYTQ | | PVTKSFNRGEC | |
| | KSLSLSPGK | | | |
| 59 | EVQLLESGGGLVQPGGSLRLSCAASGFTFS | 1685 | DIQMTQSPSSLSASV | 1773 |
| | NAWMSWVRQAPGKGLEWVADISYDGTN | | GDRVTITCRASQSISS | |
| | DYYADSVKGRFTISRDNSKNTLYLQMNSL | | YLNWYQQKPGKAP | |
| | RAEDTAVYYCTTEELRFGGFDYWGQGTL | | KLLIYDASNLETGVP | |
| | VTVSSSTKGPSVFPLAPSSKSTSGGTAALG | | SRFSGSGSGTDFTLTI | |
| | CLVKDYFPEPVTVSWNSGALTSGVHTFPA | | SSLQPEDFATYYCQQ | |
| | VLQSSGLYSLSSVVTVPSSSLGTQTYICNV | | ANSFPLTFGQGTKVE | |
| | NHKPSNTKVDKKVDKTHTCPPCPPELLGG | | IKRTVAAPSVFIFPPS | |
| | PSVFLFPPKPKDTLMISRTPEVTCVVVDVS | | DEQLKSGTASVVCL | |
| | HEDPEVKFNWYVDGVEVHNAKTKPREEQ | | LNNFYPREAKVQWK | |
| | YNSTYRVVSVLTVLHQDWLNGKEYKCKV | | VDNALQSGNSQESV | |
| | SNKALPAPIEKTISKAKQPREPQVYTLPPSR | | TEQDSKDSTYSLSST | |
| | EEMTKNQVSLTCLVKGFYPSDIAVEWESN | | LTLSKADYEKHKVY | |
| | GQPENNYKTTPPVLDSDGSFFLYSKLTVD | | ACEVTHQGLSSPVT | |
| | KSRWQQGNVFSCSVMHEALHNHYTQKSL | | KSFNRGEC | |
| | SLSPGK | | | |
| 60 | QVQLVQSGAEVKKPGSSVKVSCKASGGTF | 1686 | EIVMTQSPATLSVSP | 1774 |
| | SSYAISWVRQAPGQGLEWMGGIIPMFGTA | | GERATLSCRASQSIG | |
| | NYAQKFQGRVTITADESTSTAYMELSSLRS | | TYLAWYQQKPGQAP | |
| | EDTAVYYCARDLGYSNAGGTLHYWGQGT | | RLLIYDASSRATGIP | |
| | LVTVSSSTKGPSVFPLAPSSKSTSGGTAAL | | ARFSGSGSGTEFTLTI | |
| | GCLVKDYFPEPVTVSWNSGALTSGVHTFP | | SSLQSEDFAVYYCQ | |
| | AVLQSSGLYSLSSVVTVPSSSLGTQTYICN | | QYKSYPLTFGGGTK | |
| | VNHKPSNTKVDKKVDKTHTCPPCPPELLG | | VEIKRTVAAPSVFIFP | |
| | GPSVFLFPPKPKDTLMISRTPEVTCVVVDV | | PSDEQLKSGTASVVC | |
| | SHEDPEVKFNWYVDGVEVHNAKTKPREE | | LLNNFYPREAKVQW | |
| | QYNSTYRVVSVLTVLHQDWLNGKEYKCK | | KVDNALQSGNSQES | |
| | VSNKALPAPIEKTISKAKQPREPQVYTLPPS | | VTEQDSKDSTYSLSS | |
| | REEMTKNQVSLTCLVKGFYPSDIAVEWES | | TLTLSKADYEKHKV | |
| | NGQPENNYKTTPPVLDSDGSFFLYSKLTV | | YACEVTHQGLSSPV | |
| | DKSRWQQGNVFSCSVMHEALHNHYTQKS | | TKSFNRGEC | |
| | LSLSPGK | | | |
| 61 | QVQLVQSGAEVKKPGASVKVSCKASGYT | 1687 | DIQMTQSPSSLSASV | 1775 |
| | FTNYYMHWVRQAPGQGLEWMGIINPSGG | | GDRVTITCQASQDIS | |
| | STSYAQKFQGRVTMTRDTSTSTVYMELSS | | NYLNWYQQKPGKA | |
| | LRSEDTAVYYCARAEWDILTGYYIDYWG | | PKLLIYGASSLQSGV | |
| | QGTLVTVSSSTKGPSVFPLAPSSKSTSGGT | | PSRFSGSGSGTDFTL | |
| | AALGCLVKDYFPEPVTVSWNSGALTSGVH | | TISSLQPEDFATYYC | |
| | TFPAVLQSSGLYSLSSVVTVPSSSLGTQTYI | | QQHNSYPWTFGQGT | |
| | CNVNHKPSNTKVDKKVDKTHTCPPCPPEL | | KVEIKRTVAAPSVFI | |
| | LGGPSVFLFPPKPKDTLMISRTPEVTCVVV | | FPPSDEQLKSGTASV | |
| | DVSHEDPEVKFNWYVDGVEVHNAKTKPR | | VCLLNNFYPREAKV | |
| | EEQYNSTYRVVSVLTVLHQDWLNGKEYK | | QWKVDNALQSGNS | |
| | CKVSNKALPAPIEKTISKAKQPREPQVYTL | | QESVTEQDSKDSTYS | |
| | PPSREEMTKNQVSLTCLVKGFYPSDIAVE | | LSSTLTLSKADYEKH | |
| | WESNGQPENNYKTTPPVLDSDGSFFLYSK | | KVYACEVTHQGLSS | |
| | LTVDKSRWQQGNVFSCSVMHEALHNHYT | | PVTKSFNRGEC | |
| | QKSLSLSPGK | | | |
| 62 | QVQLVQSGAEVKKPGASVKVSCKASGYT | 1688 | DIQMTQSPSSLSASV | 1776 |
| | FTDHFVHWVRQAPGQGLEWMGWISAYN | | GDRVTITCRASQGIH | |
| | GNTNYAQKFQGRVTMTRDTSTSTVYMEL | | NYLAWYQQKPGKA | |
| | SSLRSEDTAVYYCARAEYSYGFDYWGQG | | PKLLIYDASNLETGV | |
| | TLVTVSSSTKGPSVFPLAPSSKSTSGGTAA | | PSRFSGSGSGTDFTL | |
| | LGCLVKDYFPEPVTVSWNSGALTSGVHTF | | TISSLQPEDFATYYC | |
| | PAVLQSSGLYSLSSVVTVPSSSLGTQTYIC | | QQTSSFPYTFGQGTK | |
| | NVNHKPSNTKVDKKVDKTHTCPPCPPELL | | LEIKRTVAAPSVFIFP | |
| | GGPSVFLFPPKPKDTLMISRTPEVTCVVVD | | PSDEQLKSGTASVVC | |
| | VSHEDPEVKFNWYVDGVEVHNAKTKPRE | | LLNNFYPREAKVQW | |
| | EQYNSTYRVVSVLTVLHQDWLNGKEYKC | | KVDNALQSGNSQES | |
| | KVSNKALPAPIEKTISKAKQPREPQVYTLP | | VTEQDSKDSTYSLSS | |
| | PSREEMTKNQVSLTCLVKGFYPSDIAVEW | | TLTLSKADYEKHKV | |
| | ESNGQPENNYKTTPPVLDSDGSFFLYSKLT | | YACEVTHQGLSSPV | |
| | VDKSRWQQGNVFSCSVMHEALHNHYTQ | | TKSFNRGEC | |
| | KSLSLSPGK | | | |
| 63 | QVQLVQSGAEVKKPGASVKVSCKASGYT | 1689 | DIQMTQSPSSLSASV | 1777 |
| | FTGYYVHWVRQAPGQGLEWMGVINPSGG | | GDRVTITCQASQDIS | |
| | GSPSYAQKFQGRVTMTRDTSTSTVYMELS | | NYLNWYQQKPGKA | |
| | SLRSEDTAVYYCARDRSDVDYGMDVWG | | PKLLIYDASNLQSGV | |
| | QGTTVTVSSSTKGPSVFPLAPSSKSTSGGT | | PSRFSGSGSGTDFTL | |
| | AALGCLVKDYFPEPVTVSWNSGALTSGVH | | TISSLQPEDFATYYC | |
| | TFPAVLQSSGLYSLSSVVTVPSSSLGTQTYI | | LQHNSYPLTFGGGT | |
| | CNVNHKPSNTKVDKKVDKTHTCPPCPPEL | | KVEIKRTVAAPSVFI | |
| | LGGPSVFLFPPKPKDTLMISRTPEVTCVVV | | FPPSDEQLKSGTASV | |
| | DVSHEDPEVKFNWYVDGVEVHNAKTKPR | | VCLLNNFYPREAKV | |
| | EEQYNSTYRVVSVLTVLHQDWLNGKEYK | | QWKVDNALQSGNS | |
| | CKVSNKALPAPIEKTISKAKQPREPQVYTL | | QESVTEQDSKDSTYS | |
| | PPSREEMTKNQVSLTCLVKGFYPSDIAVE | | LSSTLTLSKADYEKH | |
| | WESNGQPENNYKTTPPVLDSDGSFFLYSK | | KVYACEVTHQGLSS | |
| | LTVDKSRWQQGNVFSCSVMHEALHNHYT | | PVTKSFNRGEC | |
| | QKSLSLSPGK | | | |
| 64 | QVQLVQSGAEVKKPGASVKVSCKASGYT | 1690 | DIVMTQSPLSLPVTP | 1778 |
| | FTDYYMHWVRQAPGQGLEWMGLIDPSGG | | GEPASISCRSSQSLLH | |
| | STNSLQKFQGRVTMTRDTSTSTVYMELSS | | SNGYNYLDWYLQKP | |
| | LRSEDTAVYYCARDVGFGELSFDIWGQGT | | GQSPQLLIYAASTLQ | |
| | TVTVSSSTKGPSVFPLAPSSKSTSGGTAAL | | SGVPDRFSGSGSGTD | |
| | GCLVKDYFPEPVTVSWNSGALTSGVHTFP | | FTLKISRVEAEDVGV | |
| | AVLQSSGLYSLSSVVTVPSSSLGTQTYICN | | YYCMQGTHWPPTFG | |
| | VNHKPSNTKVDKKVDKTHTCPPCPPELLG | | PGTKVDIKRTVAAPS | |
| | GPSVFLFPPKPKDTLMISRTPEVTCVVVDV | | VFIFPPSDEQLKSGT | |
| | SHEDPEVKFNWYVDGVEVHNAKTKPREE | | ASVVCLLNNFYPRE | |
| | QYNSTYRVVSVLTVLHQDWLNGKEYKCK | | AKVQWKVDNALQS | |
| | VSNKALPAPIEKTISKAKQPREPQVYTLPPS | | GNSQESVTEQDSKD | |
| | REEMTKNQVSLTCLVKGFYPSDIAVEWES | | STYSLSSTLTLSKAD | |
| | NGQPENNYKTTPPVLDSDGSFFLYSKLTV | | YEKHKVYACEVTHQ | |
| | DKSRWQQGNVFSCSVMHEALHNHYTQKS | | GLSSPVTKSFNRGEC | |
| | LSLSPGK | | | |
| 65 | QVQLVQSGAEVKKPGASVKVSCKASGYT | 1691 | DIQMTQSPSSLSASV | 1779 |
| | FTGYYMHWVRQAPGQGLEWMGWINPNS | | GDRVTITCRASQSIG | |
| | GGTNYAQKFQGRVTMTRDTSTSTVYMEL | | TYLNWYQQKPGKAP | |
| | SSLRSEDTAVYYCAREIGGYDNYYYYGM | | KLLIYAASSLQSGVP | |
| | DVWGQGTTVTVSSSTKGPSVFPLAPSSKST | | SRFSGSGSGTDFTLTI | |
| | SGGTAALGCLVKDYFPEPVTVSWNSGALT | | SSLQPEDFATYYCQQ | |
| | SGVHTFPAVLQSSGLYSLSSVVTVPSSSLG | | SYTDPWTFGQGTKV | |
| | TQTYICNVNHKPSNTKVDKKVDKTHTCPP | | EIKRTVAAPSVFIFPP | |
| | CPPELLGGPSVFLFPPKPKDTLMISRTPEVT | | SDEQLKSGTASVVC | |
| | CVVVDVSHEDPEVKFNWYVDGVEVHNA | | LLNNFYPREAKVQW | |
| | KTKPREEQYNSTYRVVSVLTVLHQDWLN | | KVDNALQSGNSQES | |
| | GKEYKCKVSNKALPAPIEKTISKAKQPREP | | VTEQDSKDSTYSLSS | |
| | QVYTLPPSREEMTKNQVSLTCLVKGFYPS | | TLTLSKADYEKHKV | |
| | DIAVEWESNGQPENNYKTTPPVLDSDGSF | | YACEVTHQGLSSPV | |
| | FLYSKLTVDKSRWQQGNVFSCSVMHEAL | | TKSFNRGEC | |
| | HNHYTQKSLSLSPGK | | | |
| 66 | QVQLVQSGAEVKKPGASVKVSCKASGYT | 1692 | DIQMTQSPSSLSASV | 1780 |
| | FNTYYMHWVRQAPGQGLEWMGWMHPN | | GDRVTITCRASQSIFS | |
| | TGNTGYAQKFQGRVTMTRDTSTSTVYME | | YLNWYQQKPGKAP | |
| | LSSLRSEDTAVYYCARGTTSDAFDIWGQG | | KLLIYSASNLQSGVP | |
| | TMVTVSSSTKGPSVFPLAPSSKSTSGGTAA | | SRFSGSGSGTDFTLTI | |
| | LGCLVKDYFPEPVTVSWNSGALTSGVHTF | | SSLQPEDFATYYCQQ | |
| | PAVLQSSGLYSLSSVVTVPSSSLGTQTYIC | | SYSTPITFGQGTKVEI | |
| | NVNHKPSNTKVDKKVDKTHTCPPCPPELL | | KRTVAAPSVFIFPPS | |
| | GGPSVFLFPPKPKDTLMISRTPEVTCVVVD | | DEQLKSGTASVVCL | |
| | VSHEDPEVKFNWYVDGVEVHNAKTKPRE | | LNNFYPREAKVQWK | |
| | EQYNSTYRVVSVLTVLHQDWLNGKEYKC | | VDNALQSGNSQESV | |
| | KVSNKALPAPIEKTISKAKQPREPQVYTLP | | TEQDSKDSTYSLSST | |
| | PSREEMTKNQVSLTCLVKGFYPSDIAVEW | | LTLSKADYEKHKVY | |
| | ESNGQPENNYKTTPPVLDSDGSFFLYSKLT | | ACEVTHQGLSSPVT | |
| | VDKSRWQQGNVFSCSVMHEALHNHYTQ | | KSFNRGEC | |
| | KSLSLSPGK | | | |
| 67 | QVQLVQSGAEVKKPGASVKVSCKASGDT | 1693 | DIQMTQSPSSLSASV | 1781 |
| | FTRHYVHWVRQAPGQGLEWMGRVNPRD | | GDRVTITCRASQGIS | |
| | GRTNSAQKFQGRVTMTRDTSTSTVYMELS | | SYLAWYQQKPGKAP | |
| | SLRSEDTAVYYCAKDMFPTVTGTYYYYG | | KLLIYDASNLETGVP | |
| | MDVWGQGTTVTVSSSTKGPSVFPLAPSSK | | SRFSGSGSGTDFTLTI | |
| | STSGGTAALGCLVKDYFPEPVTVSWNSGA | | SSLQPEDFATYYCQQ | |
| | LTSGVHTFPAVLQSSGLYSLSSVVTVPSSS | | ASGFPYTFGQGTRLE | |
| | LGTQTYICNVNHKPSNTKVDKKVDKTHTC | | IKRTVAAPSVFIFPPS | |
| | PPCPPELLGGPSVFLFPPKPKDTLMISRTPE | | DEQLKSGTASVVCL | |
| | VTCVVVDVSHEDPEVKFNWYVDGVEVHN | | LNNFYPREAKVQWK | |
| | AKTKPREEQYNSTYRVVSVLTVLHQDWL | | VDNALQSGNSQESV | |
| | NGKEYKCKVSNKALPAPIEKTISKAKQPRE | | TEQDSKDSTYSLSST | |
| | PQVYTLPPSREEMTKNQVSLTCLVKGFYP | | LTLSKADYEKHKVY | |
| | SDIAVEWESNGQPENNYKTTPPVLDSDGS | | ACEVTHQGLSSPVT | |
| | FFLYSKLTVDKSRWQQGNVFSCSVMHEA | | KSFNRGEC | |
| | LHNHYTQKSLSLSPGK | | | |
| 68 | QVQLVQSGAEVKKPGASVKVSCKASGYT | 1694 | DIQMTQSPSSLSASV | 1782 |
| | FSSYDINWVRQAPGQGLEWVGWINPRNG | | GDRVTITCRASQSIS | |
| | GTDYAQKFQGRVTMTRDTSTSTVYMELSS | | NYLNWYQQKPGKA | |
| | LRSEDTAVYYCARHRWELDSFDYWGQGT | | PKLLIYATSSLQSGV | |
| | LVTVSSSTKGPSVFPLAPSSKSTSGGTAAL | | PSRFSGSGSGTDFTL | |
| | GCLVKDYFPEPVTVSWNSGALTSGVHTFP | | TISSLQPEDFATYYC | |
| | AVLQSSGLYSLSSVVTVPSSSLGTQTYICN | | QQGYNIPFTFGQGTK | |
| | VNHKPSNTKVDKKVDKTHTCPPCPPELLG | | LEIKRTVAAPSVFIFP | |
| | GPSVFLFPPKPKDTLMISRTPEVTCVVVDV | | PSDEQLKSGTASVVC | |
| | SHEDPEVKFNWYVDGVEVHNAKTKPREE | | LLNNFYPREAKVQW | |
| | QYNSTYRVVSVLTVLHQDWLNGKEYKCK | | KVDNALQSGNSQES | |
| | VSNKALPAPIEKTISKAKQPREPQVYTLPPS | | VTEQDSKDSTYSLSS | |
| | REEMTKNQVSLTCLVKGFYPSDIAVEWES | | TLTLSKADYEKHKV | |
| | NGQPENNYKTTPPVLDSDGSFFLYSKLTV | | YACEVTHQGLSSPV | |
| | DKSRWQQGNVFSCSVMHEALHNHYTQKS | | TKSFNRGEC | |
| | LSLSPGK | | | |
| 69 | QVQLVQSGAEVKKPGASVKVSCKASGYT | 1695 | DIQMTQSPSSLSASV | 1783 |
| | FTSYYIHWVRQAPGQGLEWMGWMNPND | | GDRVTITCRASESISG | |
| | GKTAYAQRFQGRVTMTRDTSTSTVYMEL | | WLAWYQQKPGKAP | |
| | SSLRSEDTAVYYCARDDDYGGYVAYWGQ | | KLLIYDASNLETGVP | |
| | GTLVTVSSSTKGPSVFPLAPSSKSTSGGTA | | SRFSGSGSGTDFTLTI | |
| | ALGCLVKDYFPEPVTVSWNSGALTSGVHT | | SSLQPEDFATYYCQQ | |
| | FPAVLQSSGLYSLSSVVTVPSSSLGTQTYIC | | YDTWPFTFGPGTKV | |
| | NVNHKPSNTKVDKKVDKTHTCPPCPPELL | | DIKRTVAAPSVFIFPP | |
| | GGPSVFLFPPKPKDTLMISRTPEVTCVVVD | | SDEQLKSGTASVVC | |
| | VSHEDPEVKFNWYVDGVEVHNAKTKPRE | | LLNNFYPREAKVQW | |
| | EQYNSTYRVVSVLTVLHQDWLNGKEYKC | | KVDNALQSGNSQES | |
| | KVSNKALPAPIEKTISKAKQPREPQVYTLP | | VTEQDSKDSTYSLSS | |
| | PSREEMTKNQVSLTCLVKGFYPSDIAVEW | | TLTLSKADYEKHKV | |
| | ESNGQPENNYKTTPPVLDSDGSFFLYSKLT | | YACEVTHQGLSSPV | |
| | VDKSRWQQGNVFSCSVMHEALHNHYTQ | | TKSFNRGEC | |
| | KSLSLSPGK | | | |
| 70 | EVQLLESGGGLVQPGGSLRLSCAASGMSV | 1696 | DIQMTQSPSSLSASV | 1784 |
| | TSNHMSWVRQAPGKGLEWVSSIYPDGKT | | GDRVTITCQASQSIS | |
| | YYADSVKGRFTISRDNSKNTLYLQMNSLR | | NWLAWYQQKPGKA | |
| | AEDTAVYYCARDEEDWFDPWGQGTLVTV | | PKLLIYAASTLQSGV | |
| | SSSTKGPSVFPLAPSSKSTSGGTAALGCLV | | PSRFSGSGSGTDFTL | |
| | KDYFPEPVTVSWNSGALTSGVHTFPAVLQ | | TISSLQPEDFATYYC | |
| | SSGLYSLSSVVTVPSSSLGTQTYICNVNHK | | QQSYSTPWTFGQGT | |
| | PSNTKVDKKVDKTHTCPPCPPELLGGPSVF | | KVEIKRTVAAPSVFI | |
| | LFPPKPKDTLMISRTPEVTCVVVDVSHEDP | | FPPSDEQLKSGTASV | |
| | EVKFNWYVDGVEVHNAKTKPREEQYNST | | VCLLNNFYPREAKV | |
| | YRVVSVLTVLHQDWLNGKEYKCKVSNKA | | QWKVDNALQSGNS | |
| | LPAPIEKTISKAKQPREPQVYTLPPSREEMT | | QESVTEQDSKDSTYS | |
| | KNQVSLTCLVKGFYPSDIAVEWESNGQPE | | LSSTLTLSKADYEKH | |
| | NNYKTTPPVLDSDGSFFLYSKLTVDKSRW | | KVYACEVTHQGLSS | |
| | QQGNVFSCSVMHEALHNHYTQKSLSLSPG | | PVTKSFNRGEC | |
| | K | | | |
| 71 | EVQLLESGGGLVQPGGSLRLSCAASGFTFS | 1697 | DIQMTQSPSSLSASV | 1785 |
| | NHYMSWVRQAPGKGLEWVAVIWPDGSK | | GDRVTITCQASQDIS | |
| | EYYADSVKGRFTISRDNSKNTLYLQMNSL | | NYLNWYQQKPGKA | |
| | RAEDTAVYYCAREDYYGSGMDYWGQGT | | PKLLIYGASTLQSGV | |
| | LVTVSSSTKGPSVFPLAPSSKSTSGGTAAL | | PSRFSGSGSGTDFTL | |
| | GCLVKDYFPEPVTVSWNSGALTSGVHTFP | | TISSLQPEDFATYYC | |
| | AVLQSSGLYSLSSVVTVPSSSLGTQTYICN | | QQYDSYPPTFGGGT | |
| | VNHKPSNTKVDKKVDKTHTCPPCPPELLG | | KVEIKRTVAAPSVFI | |
| | GPSVFLFPPKPKDTLMISRTPEVTCVVVDV | | FPPSDEQLKSGTASV | |
| | SHEDPEVKFNWYVDGVEVHNAKTKPREE | | VCLLNNFYPREAKV | |
| | QYNSTYRVVSVLTVLHQDWLNGKEYKCK | | QWKVDNALQSGNS | |
| | VSNKALPAPIEKTISKAKQPREPQVYTLPPS | | QESVTEQDSKDSTYS | |
| | REEMTKNQVSLTCLVKGFYPSDIAVEWES | | LSSTLTLSKADYEKH | |
| | NGQPENNYKTTPPVLDSDGSFFLYSKLTV | | KVYACEVTHQGLSS | |
| | DKSRWQQGNVFSCSVMHEALHNHYTQKS | | PVTKSFNRGEC | |
| | LSLSPGK | | | |
| 72 | QVQLVQSGAEVKKPGASVKVSCKASGGT | 1698 | DIQMTQSPSSLSASV | 1786 |
| | FSNYAISWVRQAPGQGLEWMGWISAYNG | | GDRVTITCQASEDIN | |
| | NSDYAQNLQGRVTMTRDTSTSTVYMELSS | | KYLNWYQQKPGKA | |
| | LRSEDTAVYYCAIGDYFDYWGQGTLVTVS | | PKLLIYDASNLETGV | |
| | SSTKGPSVFPLAPSSKSTSGGTAALGCLVK | | PSRFSGSGSGTDFTL | |
| | DYFPEPVTVSWNSGALTSGVHTFPAVLQS | | TISSLQPEDFATYYC | |
| | SGLYSLSSVVTVPSSSLGTQTYICNVNHKP | | QQANSFPLTFGQGT | |
| | SNTKVDKKVDKTHTCPPCPPELLGGPSVFL | | KVEIKRTVAAPSVFI | |
| | FPPKPKDTLMISRTPEVTCVVVDVSHEDPE | | FPPSDEQLKSGTASV | |
| | VKFNWYVDGVEVHNAKTKPREEQYNSTY | | VCLLNNFYPREAKV | |
| | RVVSVLTVLHQDWLNGKEYKCKVSNKAL | | QWKVDNALQSGNS | |
| | PAPIEKTISKAKQPREPQVYTLPPSREEMTK | | QESVTEQDSKDSTYS | |
| | NQVSLTCLVKGFYPSDIAVEWESNGQPEN | | LSSTLTLSKADYEKH | |
| | NYKTTPPVLDSDGSFFLYSKLTVDKSRWQ | | KVYACEVTHQGLSS | |
| | QGNVFSCSVMHEALHNHYTQKSLSLSPGK | | PVTKSFNRGEC | |
| 73 | EVQLLESGGGLVQPGGSLRLSCAASGFTV | 1699 | DIQMTQSPSSLSASV | 1787 |
| | SSNYMSWVRQAPGKGLEWVAVIYSDGKT | | GDRVTITCRASQSIST | |
| | YYADSVKGRFTISRDNSKNTLYLQMNSLR | | YLNWYQQKPGKAP | |
| | AEDTAVYYCAREDSSGSHFDYWGQGTLV | | KLLIYDASNLETGVP | |
| | TVSSSTKGPSVFPLAPSSKSTSGGTAALGC | | SRFSGSGSGTDFTLTI | |
| | LVKDYFPEPVTVSWNSGALTSGVHTFPAV | | SSLQPEDFATYYCQQ | |
| | LQSSGLYSLSSVVTVPSSSLGTQTYICNVN | | AHSFPPTFGQGTRLE | |
| | HKPSNTKVDKKVDKTHTCPPCPPELLGGP | | IKRTVAAPSVFIFPPS | |
| | SVFLFPPKPKDTLMISRTPEVTCVVVDVSH | | DEQLKSGTASVVCL | |
| | EDPEVKFNWYVDGVEVHNAKTKPREEQY | | LNNFYPREAKVQWK | |
| | NSTYRVVSVLTVLHQDWLNGKEYKCKVS | | VDNALQSGNSQESV | |
| | NKALPAPIEKTISKAKQPREPQVYTLPPSRE | | TEQDSKDSTYSLSST | |
| | EMTKNQVSLTCLVKGFYPSDIAVEWESNG | | LTLSKADYEKHKVY | |
| | QPENNYKTTPPVLDSDGSFFLYSKLTVDKS | | ACEVTHQGLSSPVT | |
| | RWQQGNVFSCSVMHEALHNHYTQKSLSL | | KSFNRGEC | |
| | SPGK | | | |
| 74 | QVQLVQSGAEVKKPGSSVKVSCKASGYTF | 1700 | DIQMTQSPSSLSASV | 1788 |
| | TKYEINWVRQAPGQGLEWMGGIIPIFGTA | | GDRVTITCRASQGIS | |
| | NYAQKFQGRVTITADESTSTAYMELSSLRS | | NNLNWYQQKPGKA | |
| | EDTAVYYCARGSGWYTPLFDYWGQGTLV | | PKLLIYDASYLETGV | |
| | TVSSSTKGPSVFPLAPSSKSTSGGTAALGC | | PSRFSGSGSGTDFTL | |
| | LVKDYFPEPVTVSWNSGALTSGVHTFPAV | | TISSLQPEDFATYYC | |
| | LQSSGLYSLSSVVTVPSSSLGTQTYICNVN | | QQSYSAPLTFGQGT | |
| | HKPSNTKVDKKVDKTHTCPPCPPELLGGP | | KVEIKRTVAAPSVFI | |
| | SVFLFPPKPKDTLMISRTPEVTCVVVDVSH | | FPPSDEQLKSGTASV | |
| | EDPEVKFNWYVDGVEVHNAKTKPREEQY | | VCLLNNFYPREAKV | |
| | NSTYRVVSVLTVLHQDWLNGKEYKCKVS | | QWKVDNALQSGNS | |
| | NKALPAPIEKTISKAKQPREPQVYTLPPSRE | | QESVTEQDSKDSTYS | |
| | EMTKNQVSLTCLVKGFYPSDIAVEWESNG | | LSSTLTLSKADYEKH | |
| | QPENNYKTTPPVLDSDGSFFLYSKLTVDKS | | KVYACEVTHQGLSS | |
| | RWQQGNVFSCSVMHEALHNHYTQKSLSL | | PVTKSFNRGEC | |
| | SPGK | | | |
| 75 | QVQLVQSGAEVKKPGASVKVSCKASGYT | 1701 | EIVMTQSPATLSVSP | 1789 |
| | FTDYYIHWVRQAPGQGLEWMGLIDPSGGS | | GERATLSCRASQSVS | |
| | TSIAQKFQGRVTMTRDTSTSTVYMELSSLR | | SYLAWYQQKPGQAP | |
| | SEDTAVYYCARDYDILTGSGFDPWGQGTL | | RLLIYDASARATGIP | |
| | VTVSSSTKGPSVFPLAPSSKSTSGGTAALG | | ARFSGSGSGTEFTLTI | |
| | CLVKDYFPEPVTVSWNSGALTSGVHTFPA | | SSLQSEDFAVYYCQ | |
| | VLQSSGLYSLSSVVTVPSSSLGTQTYICNV | | QYRSSVTFGQGTRLE | |
| | NHKPSNTKVDKKVDKTHTCPPCPPELLGG | | IKRTVAAPSVFIFPPS | |
| | PSVFLFPPKPKDTLMISRTPEVTCVVVDVS | | DEQLKSGTASVVCL | |
| | HEDPEVKFNWYVDGVEVHNAKTKPREEQ | | LNNFYPREAKVQWK | |
| | YNSTYRVVSVLTVLHQDWLNGKEYKCKV | | VDNALQSGNSQESV | |
| | SNKALPAPIEKTISKAKQPREPQVYTLPPSR | | TEQDSKDSTYSLSST | |
| | EEMTKNQVSLTCLVKGFYPSDIAVEWESN | | LTLSKADYEKHKVY | |
| | GQPENNYKTTPPVLDSDGSFFLYSKLTVD | | ACEVTHQGLSSPVT | |
| | KSRWQQGNVFSCSVMHEALHNHYTQKSL | | KSFNRGEC | |
| | SLSPGK | | | |
| 76 | QVQLVQSGAEVKKPGASVKVSCKASGYT | 1702 | DIQMTQSPSSLSASV | 1790 |
| | FTTYYMHWVRQAPGQGLEWMGIINVSAG | | GDRVTITCQASQDIN | |
| | TTSYAQKFQGRVTMTRDTSTSTVYMELSS | | NYLNWYQQKPGKA | |
| | LRSEDTAVYYCAKEPYPHQSGWFFDYWG | | PKLLIYDASNLETGV | |
| | QGTLVTVSSSTKGPSVFPLAPSSKSTSGGT | | PSRFSGSGSGTDFTL | |
| | AALGCLVKDYFPEPVTVSWNSGALTSGVH | | TISSLQPEDFATYYC | |
| | TFPAVLQSSGLYSLSSVVTVPSSSLGTQTYI | | QQANSFPLTFGGGT | |
| | CNVNHKPSNTKVDKKVDKTHTCPPCPPEL | | KVEIKRTVAAPSVFI | |
| | LGGPSVFLFPPKPKDTLMISRTPEVTCVVV | | FPPSDEQLKSGTASV | |
| | DVSHEDPEVKFNWYVDGVEVHNAKTKPR | | VCLLNNFYPREAKV | |
| | EEQYNSTYRVVSVLTVLHQDWLNGKEYK | | QWKVDNALQSGNS | |
| | CKVSNKALPAPIEKTISKAKQPREPQVYTL | | QESVTEQDSKDSTYS | |
| | PPSREEMTKNQVSLTCLVKGFYPSDIAVE | | LSSTLTLSKADYEKH | |
| | WESNGQPENNYKTTPPVLDSDGSFFLYSK | | KVYACEVTHQGLSS | |
| | LTVDKSRWQQGNVFSCSVMHEALHNHYT | | PVTKSFNRGEC | |
| | QKSLSLSPGK | | | |
| 77 | QVQLVQSGAEVKKPGASVKVSCKASGYT | 1703 | EIVMTQSPATLSVSP | 1791 |
| | FTGHYMHWVRQAPGQGLEWMGWISTDN | | GERATLSCSASQSVG | |
| | GNANYAQKFQGRVTMTRDTSTSTVYMEL | | SSYFAWYQQKPGQA | |
| | SSLRSEDTAVYYCARDTADYYFDYWGQG | | PRLLIYDVSTRATGIP | |
| | TLVTVSSSTKGPSVFPLAPSSKSTSGGTAA | | ARFSGSGSGTEFTLTI | |
| | LGCLVKDYFPEPVTVSWNSGALTSGVHTF | | SSLQSEDFAVYYCQ | |
| | PAVLQSSGLYSLSSVVTVPSSSLGTQTYIC | | QYYSTPLTFGPGTKV | |
| | NVNHKPSNTKVDKKVDKTHTCPPCPPELL | | DIKRTVAAPSVFIFPP | |
| | GGPSVFLFPPKPKDTLMISRTPEVTCVVVD | | SDEQLKSGTASVVC | |
| | VSHEDPEVKFNWYVDGVEVHNAKTKPRE | | LLNNFYPREAKVQW | |
| | EQYNSTYRVVSVLTVLHQDWLNGKEYKC | | KVDNALQSGNSQES | |
| | KVSNKALPAPIEKTISKAKQPREPQVYTLP | | VTEQDSKDSTYSLSS | |
| | PSREEMTKNQVSLTCLVKGFYPSDIAVEW | | TLTLSKADYEKHKV | |
| | ESNGQPENNYKTTPPVLDSDGSFFLYSKLT | | YACEVTHQGLSSPV | |
| | VDKSRWQQGNVFSCSVMHEALHNHYTQ | | TKSFNRGEC | |
| | KSLSLSPGK | | | |
| 78 | QVQLVQSGAEVKKPGSSVKVSCKASGGTF | 1704 | DIQMTQSPSSLSASV | 1792 |
| | SRYPFSWVRQAPGQGLEWMGWMNPNNG | | GDRVTITCQASQDIS | |
| | DTGYAQKFQGRVTITADESTSTAYMELSS | | NYLNWYQQKPGKA | |
| | LRSEDTAVYYCARGDYPYMDVWGKGTT | | PKLLIYDASNLETGV | |
| | VTVSSSTKGPSVFPLAPSSKSTSGGTAALG | | PSRFSGSGSGTDFTL | |
| | CLVKDYFPEPVTVSWNSGALTSGVHTFPA | | TISSLQPEDFATYYC | |
| | VLQSSGLYSLSSVVTVPSSSLGTQTYICNV | | QQSYSIPYTFGQGTK | |
| | NHKPSNTKVDKKVDKTHTCPPCPPELLGG | | LEIKRTVAAPSVFIFP | |
| | PSVFLFPPKPKDTLMISRTPEVTCVVVDVS | | PSDEQLKSGTASVVC | |
| | HEDPEVKFNWYVDGVEVHNAKTKPREEQ | | LLNNFYPREAKVQW | |
| | YNSTYRVVSVLTVLHQDWLNGKEYKCKV | | KVDNALQSGNSQES | |
| | SNKALPAPIEKTISKAKQPREPQVYTLPPSR | | VTEQDSKDSTYSLSS | |
| | EEMTKNQVSLTCLVKGFYPSDIAVEWESN | | TLTLSKADYEKHKV | |
| | GQPENNYKTTPPVLDSDGSFFLYSKLTVD | | YACEVTHQGLSSPV | |
| | KSRWQQGNVFSCSVMHEALHNHYTQKSL | | TKSFNRGEC | |
| | SLSPGK | | | |
| 79 | QVQLVQSGAEVKKPGASVKVSCKASGYT | 1705 | DIQMTQSPSSLSASV | 1793 |
| | FTSDYMHWVRQAPGQGLEWMGWMNPNS | | GDRVTITCRASQGIR | |
| | GGTNYAQKFQGRVTMTRDTSTSTVYMEL | | NDLGWYQQKPGKA | |
| | SSLRSEDTAVYYCARDYITGPSDWGQGTL | | PKLLIYAASSLQPGV | |
| | VTVSSSTKGPSVFPLAPSSKSTSGGTAALG | | PSRFSGSGSGTDFTL | |
| | CLVKDYFPEPVTVSWNSGALTSGVHTFPA | | TISSLQPEDFATYYC | |
| | VLQSSGLYSLSSVVTVPSSSLGTQTYICNV | | LQTNSFPWTFGQGT | |
| | NHKPSNTKVDKKVDKTHTCPPCPPELLGG | | KLEIKRTVAAPSVFIF | |
| | PSVFLFPPKPKDTLMISRTPEVTCVVVDVS | | PPSDEQLKSGTASVV | |
| | HEDPEVKFNWYVDGVEVHNAKTKPREEQ | | CLLNNFYPREAKVQ | |
| | YNSTYRVVSVLTVLHQDWLNGKEYKCKV | | WKVDNALQSGNSQE | |
| | SNKALPAPIEKTISKAKQPREPQVYTLPPSR | | SVTEQDSKDSTYSLS | |
| | EEMTKNQVSLTCLVKGFYPSDIAVEWESN | | STLTLSKADYEKHK | |
| | GQPENNYKTTPPVLDSDGSFFLYSKLTVD | | VYACEVTHQGLSSP | |
| | KSRWQQGNVFSCSVMHEALHNHYTQKSL | | VTKSFNRGEC | |
| | SLSPGK | | | |
| 80 | QVQLVQSGAEVKKPGASVKVSCKASGFTF | 1706 | DIQMTQSPSSLSASV | 1794 |
| | TSYYMHWVRQAPGQGLEWMGWMNPNS | | GDRVTITCRASQSISS | |
| | GNTGYAQRFQGRVTMTRDTSTSTVYMEL | | WLAWYQQKPGKAP | |
| | SSLRSEDTAVYYCARGHSRTDYGMDVWG | | KLLIYDTSSLQSGVP | |
| | QGTTVTVSSSTKGPSVFPLAPSSKSTSGGT | | SRFSGSGSGTDFTLTI | |
| | AALGCLVKDYFPEPVTVSWNSGALTSGVH | | SSLQPEDFATYYCQQ | |
| | TFPAVLQSSGLYSLSSVVTVPSSSLGTQTYI | | GYSTPLTFGQGTKVE | |
| | CNVNHKPSNTKVDKKVDKTHTCPPCPPEL | | IKRTVAAPSVFIFPPS | |
| | LGGPSVFLFPPKPKDTLMISRTPEVTCVVV | | DEQLKSGTASVVCL | |
| | DVSHEDPEVKFNWYVDGVEVHNAKTKPR | | LNNFYPREAKVQWK | |
| | EEQYNSTYRVVSVLTVLHQDWLNGKEYK | | VDNALQSGNSQESV | |
| | CKVSNKALPAPIEKTISKAKQPREPQVYTL | | TEQDSKDSTYSLSST | |
| | PPSREEMTKNQVSLTCLVKGFYPSDIAVE | | LTLSKADYEKHKVY | |
| | WESNGQPENNYKTTPPVLDSDGSFFLYSK | | ACEVTHQGLSSPVT | |
| | LTVDKSRWQQGNVFSCSVMHEALHNHYT | | KSFNRGEC | |
| | QKSLSLSPGK | | | |
| 81 | EVQLLESGGGLVQPGGSLRLSCAASGFTFS | 1707 | DIQMTQSPSSLSASV | 1795 |
| | DHYMSWVRQAPGKGLEWVSIIYPDGKTY | | GDRVTITCQASQDIS | |
| | YADSVKGRFTISRDNSKNTLYLQMNSLRA | | NYLNWYQQKPGKA | |
| | EDTAVYYCAREGSYGDYDGMDVWGQGT | | PKLLIYGASTLQSGV | |
| | TVTVSSSTKGPSVFPLAPSSKSTSGGTAAL | | PSRFSGSGSGTDFTL | |
| | GCLVKDYFPEPVTVSWNSGALTSGVHTFP | | TISSLQPEDFATYYC | |
| | AVLQSSGLYSLSSVVTVPSSSLGTQTYICN | | QQSYSTPWTFGQGT | |
| | VNHKPSNTKVDKKVDKTHTCPPCPPELLG | | KLEIKRTVAAPSVFIF | |
| | GPSVFLFPPKPKDTLMISRTPEVTCVVVDV | | PPSDEQLKSGTASVV | |
| | SHEDPEVKFNWYVDGVEVHNAKTKPREE | | CLLNNFYPREAKVQ | |
| | QYNSTYRVVSVLTVLHQDWLNGKEYKCK | | WKVDNALQSGNSQE | |
| | VSNKALPAPIEKTISKAKQPREPQVYTLPPS | | SVTEQDSKDSTYSLS | |
| | REEMTKNQVSLTCLVKGFYPSDIAVEWES | | STLTLSKADYEKHK | |
| | NGQPENNYKTTPPVLDSDGSFFLYSKLTV | | VYACEVTHQGLSSP | |
| | DKSRWQQGNVFSCSVMHEALHNHYTQKS | | VTKSFNRGEC | |
| | LSLSPGK | | | |
| 82 | QVQLVQSGAEVKKPGSSVKVSCKASGGTF | 1708 | EIVMTQSPATLSVSP | 1796 |
| | SNYDISWVRQAPGQGLEWMGGIIPIFGTAN | | GERATLSCRASQSVS | |
| | YAQKFQGRVTITADESTSTAYMELSSLRSE | | SYLAWYQQKPGQAP | |
| | DTAVYYCAREAEEGGWFDPWGQGTLVTV | | RLLIYGASTRATGIP | |
| | SSSTKGPSVFPLAPSSKSTSGGTAALGCLV | | ARFSGSGSGTEFTLTI | |
| | KDYFPEPVTVSWNSGALTSGVHTFPAVLQ | | SSLQSEDFAVYYCQ | |
| | SSGLYSLSSVVTVPSSSLGTQTYICNVNHK | | QYAFSPITFGQGTKL | |
| | PSNTKVDKKVDKTHTCPPCPPELLGGPSVF | | EIKRTVAAPSVFIFPP | |
| | LFPPKPKDTLMISRTPEVTCVVVDVSHEDP | | SDEQLKSGTASVVC | |
| | EVKFNWYVDGVEVHNAKTKPREEQYNST | | LLNNFYPREAKVQW | |
| | YRVVSVLTVLHQDWLNGKEYKCKVSNKA | | KVDNALQSGNSQES | |
| | LPAPIEKTISKAKQPREPQVYTLPPSREEMT | | VTEQDSKDSTYSLSS | |
| | KNQVSLTCLVKGFYPSDIAVEWESNGQPE | | TLTLSKADYEKHKV | |
| | NNYKTTPPVLDSDGSFFLYSKLTVDKSRW | | YACEVTHQGLSSPV | |
| | QQGNVFSCSVMHEALHNHYTQKSLSLSPG | | TKSFNRGEC | |
| | K | | | |
| 83 | QVQLVQSGAEVKKPGASVKVSCKASGYT | 1709 | DIQMTQSPSSLSASV | 1797 |
| | FTDYYMHWVRQAPGQGLEWMGWMNPN | | GDRVTITCRVSQGIS | |
| | SGYTAYAQKFQGRVTMTRDTSTSTVYME | | SYLNWYQQKPGKAP | |
| | LSSLRSEDTAVYYCAKDTPGSGWSSGMD | | KLLIYDASNLETGVP | |
| | VWGQGTTVTVSSSTKGPSVFPLAPSSKSTS | | SRFSGSGSGTDFTLTI | |
| | GGTAALGCLVKDYFPEPVTVSWNSGALTS | | SSLQPEDFATYYCQQ | |
| | GVHTFPAVLQSSGLYSLSSVVTVPSSSLGT | | SYSTPLTFGGGTKVE | |
| | QTYICNVNHKPSNTKVDKKVDKTHTCPPC | | IKRTVAAPSVFIFPPS | |
| | PPELLGGPSVFLFPPKPKDTLMISRTPEVTC | | DEQLKSGTASVVCL | |
| | VVVDVSHEDPEVKFNWYVDGVEVHNAKT | | LNNFYPREAKVQWK | |
| | KPREEQYNSTYRVVSVLTVLHQDWLNGK | | VDNALQSGNSQESV | |
| | EYKCKVSNKALPAPIEKTISKAKQPREPQV | | TEQDSKDSTYSLSST | |
| | YTLPPSREEMTKNQVSLTCLVKGFYPSDIA | | LTLSKADYEKHKVY | |
| | VEWESNGQPENNYKTTPPVLDSDGSFFLY | | ACEVTHQGLSSPVT | |
| | SKLTVDKSRWQQGNVFSCSVMHEALHNH | | KSFNRGEC | |
| | YTQKSLSLSPGK | | | |
| 84 | QVQLVQSGAEVKKPGASVKVSCKASGGT | 1710 | DIQMTQSPSSLSASV | 1798 |
| | FSNYAISWVRQAPGQGLEWMGWINPNSG | | GDRVTITCRASQSISS | |
| | GTNYAQKFQGRVTMTRDTSTSTVYMELSS | | WLAWYQQKPGKAP | |
| | LRSEDTAVYYCARVGYYDSSGGGMDVW | | KLLIYDASNLETGVP | |
| | GQGTTVTVSSSTKGPSVFPLAPSSKSTSGG | | SRFSGSGSGTDFTLTI | |
| | TAALGCLVKDYFPEPVTVSWNSGALTSGV | | SSLQPEDFATYYCLQ | |
| | HTFPAVLQSSGLYSLSSVVTVPSSSLGTQT | | THSFPLTFGPGTKVD | |
| | YICNVNHKPSNTKVDKKVDKTHTCPPCPP | | IKRTVAAPSVFIFPPS | |
| | ELLGGPSVFLFPPKPKDTLMISRTPEVTCV | | DEQLKSGTASVVCL | |
| | VVDVSHEDPEVKFNWYVDGVEVHNAKTK | | LNNFYPREAKVQWK | |
| | PREEQYNSTYRVVSVLTVLHQDWLNGKE | | VDNALQSGNSQESV | |
| | YKCKVSNKALPAPIEKTISKAKQPREPQVY | | TEQDSKDSTYSLSST | |
| | TLPPSREEMTKNQVSLTCLVKGFYPSDIAV | | LTLSKADYEKHKVY | |
| | EWESNGQPENNYKTTPPVLDSDGSFFLYS | | ACEVTHQGLSSPVT | |
| | KLTVDKSRWQQGNVFSCSVMHEALHNHY | | KSFNRGEC | |
| | TQKSLSLSPGK | | | |
| 85 | QVQLVQSGAEVKKPGASVKVSCKASGYT | 1711 | DIQMTQSPSSLSASV | 1799 |
| | FTGYYMHWVRQAPGQGLEWMGIINPIGG | | GDRVTITCRASQSVS | |
| | LTTYAQKFQGRVTMTRDTSTSTVYMELSS | | NWLAWYQQKPGKA | |
| | LRSEDTAVYYCASGAYGDYVDWYFDLW | | PKLLIYDASNLQTGV | |
| | GRGTLVTVSSSTKGPSVFPLAPSSKSTSGG | | PSRFSGSGSGTDFTL | |
| | TAALGCLVKDYFPEPVTVSWNSGALTSGV | | TISSLQPEDFATYYC | |
| | HTFPAVLQSSGLYSLSSVVTVPSSSLGTQT | | QQANSFPLTFGGGT | |
| | YICNVNHKPSNTKVDKKVDKTHTCPPCPP | | KLEIKRTVAAPSVFIF | |
| | ELLGGPSVFLFPPKPKDTLMISRTPEVTCV | | PPSDEQLKSGTASVV | |
| | VVDVSHEDPEVKFNWYVDGVEVHNAKTK | | CLLNNFYPREAKVQ | |
| | PREEQYNSTYRVVSVLTVLHQDWLNGKE | | WKVDNALQSGNSQE | |
| | YKCKVSNKALPAPIEKTISKAKQPREPQVY | | SVTEQDSKDSTYSLS | |
| | TLPPSREEMTKNQVSLTCLVKGFYPSDIAV | | STLTLSKADYEKHK | |
| | EWESNGQPENNYKTTPPVLDSDGSFFLYS | | VYACEVTHQGLSSP | |
| | KLTVDKSRWQQGNVFSCSVMHEALHNHY | | VTKSFNRGEC | |
| | TQKSLSLSPGK | | | |
| 86 | QVQLVQSGAEVKKPGASVKVSCKASGYT | 1712 | DIVMTQSPLSLPVTP | 1800 |
| | FTTYGISWVRQAPGQGLEWMGWINPNSG | | GEPASISCRSSRSLLH | |
| | DTNYAQKFQGRVTMTRDTSTSTVYMELSS | | SNGYNYLDWYLQKP | |
| | LRSEDTAVYYCARLTTATDSFDLWGRGTL | | GQSPQLLIYLGSYRA | |
| | VTVSSSTKGPSVFPLAPSSKSTSGGTAALG | | SGVPDRFSGSGSGTD | |
| | CLVKDYFPEPVTVSWNSGALTSGVHTFPA | | FTLKISRVEAEDVGV | |
| | VLQSSGLYSLSSVVTVPSSSLGTQTYICNV | | YYCMQGTHWPPTFG | |
| | NHKPSNTKVDKKVDKTHTCPPCPPELLGG | | QGTKLEIKRTVAAPS | |
| | PSVFLFPPKPKDTLMISRTPEVTCVVVDVS | | VFIFPPSDEQLKSGT | |
| | HEDPEVKFNWYVDGVEVHNAKTKPREEQ | | ASVVCLLNNFYPRE | |
| | YNSTYRVVSVLTVLHQDWLNGKEYKCKV | | AKVQWKVDNALQS | |
| | SNKALPAPIEKTISKAKQPREPQVYTLPPSR | | GNSQESVTEQDSKD | |
| | EEMTKNQVSLTCLVKGFYPSDIAVEWESN | | STYSLSSTLTLSKAD | |
| | GQPENNYKTTPPVLDSDGSFFLYSKLTVD | | YEKHKVYACEVTHQ | |
| | KSRWQQGNVFSCSVMHEALHNHYTQKSL | | GLSSPVTKSFNRGEC | |
| | SLSPGK | | | |
| 87 | QVQLVQSGAEVKKPGASVKVSCKASGYS | 1713 | DIQMTQSPSSLSASV | 1801 |
| | FTNYYIHWVRQAPGQGLEWMGWMNPYT | | GDRVTITCRASQSIS | |
| | GQTGYAQKFQGRVTMTRDTSTSTVYMEL | | RYLNWYQQKPGKA | |
| | SSLRSEDTAVYYCTTDEETMDFHLWGRGT | | PKLLIYDASNLETGV | |
| | LVTVSSSTKGPSVFPLAPSSKSTSGGTAAL | | PSRFSGSGSGTDFTL | |
| | GCLVKDYFPEPVTVSWNSGALTSGVHTFP | | TISSLQPEDFATYYC | |
| | AVLQSSGLYSLSSVVTVPSSSLGTQTYICN | | QQSYSTPWTFGQGT | |
| | VNHKPSNTKVDKKVDKTHTCPPCPPELLG | | KLEIKRTVAAPSVFIF | |
| | GPSVFLFPPKPKDTLMISRTPEVTCVVVDV | | PPSDEQLKSGTASVV | |
| | SHEDPEVKFNWYVDGVEVHNAKTKPREE | | CLLNNFYPREAKVQ | |
| | QYNSTYRVVSVLTVLHQDWLNGKEYKCK | | WKVDNALQSGNSQE | |
| | VSNKALPAPIEKTISKAKQPREPQVYTLPPS | | SVTEQDSKDSTYSLS | |
| | REEMTKNQVSLTCLVKGFYPSDIAVEWES | | STLTLSKADYEKHK | |
| | NGQPENNYKTTPPVLDSDGSFFLYSKLTV | | VYACEVTHQGLSSP | |
| | DKSRWQQGNVFSCSVMHEALHNHYTQKS | | VTKSFNRGEC | |
| | LSLSPGK | | | |
| 88 | QVQLVQSGAEVKKPGASVKVSCKASGYT | 1714 | DIVMTQSPLSLPVTP | 1802 |
| | FTGYHIHWVRQAPGQGLEWMGRINPNSG | | GEPASISCRSSRSLLH | |
| | GTDYAQKFQGRVTMTRDTSTSTVYMELSS | | SNGYNYLDWYLQKP | |
| | LRSEDTAVYYCARETYSGSYEESFDYWGQ | | GQSPQLLIYLGSDRA | |
| | GTLVTVSSSTKGPSVFPLAPSSKSTSGGTA | | SGVPDRFSGSGSGTD | |
| | ALGCLVKDYFPEPVTVSWNSGALTSGVHT | | FTLKISRVEAEDVGV | |
| | FPAVLQSSGLYSLSSVVTVPSSSLGTQTYIC | | YYCMQGTHWPPTFG | |
| | NVNHKPSNTKVDKKVDKTHTCPPCPPELL | | QGTKVEIKRTVAAPS | |
| | GGPSVFLFPPKPKDTLMISRTPEVTCVVVD | | VFIFPPSDEQLKSGT | |
| | VSHEDPEVKFNWYVDGVEVHNAKTKPRE | | ASVVCLLNNFYPRE | |
| | EQYNSTYRVVSVLTVLHQDWLNGKEYKC | | AKVQWKVDNALQS | |
| | KVSNKALPAPIEKTISKAKQPREPQVYTLP | | GNSQESVTEQDSKD | |
| | PSREEMTKNQVSLTCLVKGFYPSDIAVEW | | STYSLSSTLTLSKAD | |
| | ESNGQPENNYKTTPPVLDSDGSFFLYSKLT | | YEKHKVYACEVTHQ | |
| | VDKSRWQQGNVFSCSVMHEALHNHYTQ | | GLSSPVTKSFNRGEC | |
| | KSLSLSPGK | | | |

TABLE 15
IgG4 Antibodies
Antibody (mAb) Examples 89-130 target CD33, and 131-176
target CLL-1.

| mAb | | SEQ | | SEQ |
|---|---|---|---|---|
| Ex. | Heavy Chain | ID NO | Light Chain | ID NO |
| 89 | QVQLVQSGAEVKKPGASVKVSCKASGYS | 1803 | DIQMTQSPSSLSASVG | 1891 |
| | FTGYYIHWVRQAPGQGLEWMGWINPNS | | DRVTITCRASQTINDW | |
| | GGTNYAQKFQGRVTMTRDTSTSTVYMEL | | LAWYQQKPGKAPKLL | |
| | SSLRSEDTAVYYCARDQWDGYNSGYFD | | IYSASTLHSGVPSRFSG | |
| | YWGQGTLVTVSSSTKGPSVFPLAPCSRST | | SGSGTDFTLTISSLQPE | |
| | SESTAALGCLVKDYFPEPVTVSWNSGALT | | DFATYYCQQAYSTPW | |
| | SGVHTFPAVLQSSGLYSLSSVVTVPSSSLG | | TFGQGTKVEIKRTVAA | |
| | TKTYTCNVDHKPSNTKVDKRVSKYGPPC | | PSVFIFPPSDEQLKSGT | |
| | PSCPPEFLGGPSVFLFPPKPKDTLMISRTPE | | ASVVCLLNNFYPREA | |
| | VTCVVVDVSQEDPEVQFNWYVDGVEVH | | KVQWKVDNALQSGN | |
| | NAKTKPREEQFNSTYRVVSVLTVLHQDW | | SQESVTEQDSKDSTYS | |
| | LNGKEYKCKVSNKGLPSSIEKTISKAKQP | | LSSTLTLSKADYEKHK | |
| | REPQVYTLPPSQEEMTKNQVSLTCLVKGF | | VYACEVTHQGLSSPV | |
| | YPSDIAVEWESNGQPENNYKTTPPVLDSD | | TKSFNRGEC | |
| | GSFFLYSRLTVDKSRWQEGNVFSCSVMH | | | |
| | EALHNHYTQKSLSLSLGK | | | |
| 90 | EVQLLESGGGLVQPGGSLRLSCAASGFTF | 1804 | DIQMTQSPSSLSASVG | 1892 |
| | SDYYMSWVRQAPGKGLEWVSGISGSGYS | | DRVTITCRASQSISRYL | |
| | TYYADSVKGRFTISRDNSKNTLYLQMNS | | NWYQQKPGKAPKLLI | |
| | LRAEDTAVYYCARTFGRGPDWYFDLWG | | YTASTLQSGVPSRFSG | |
| | RGTLVTVSSSTKGPSVFPLAPCSRSTSEST | | SGSGTDFTLTISSLQPE | |
| | AALGCLVKDYFPEPVTVSWNSGALTSGV | | DFATYYCQQYDDLPL | |
| | HTFPAVLQSSGLYSLSSVVTVPSSSLGTKT | | TFGGGTKVEIKRTVAA | |
| | YTCNVDHKPSNTKVDKRVSKYGPPCPSC | | PSVFIFPPSDEQLKSGT | |
| | PPEFLGGPSVFLFPPKPKDTLMISRTPEVT | | ASVVCLLNNFYPREA | |
| | CVVVDVSQEDPEVQFNWYVDGVEVHNA | | KVQWKVDNALQSGN | |
| | KTKPREEQFNSTYRVVSVLTVLHQDWLN | | SQESVTEQDSKDSTYS | |
| | GKEYKCKVSNKGLPSSIEKTISKAKQPREP | | LSSTLTLSKADYEKHK | |
| | QVYTLPPSQEEMTKNQVSLTCLVKGFYPS | | VYACEVTHQGLSSPV | |
| | DIAVEWESNGQPENNYKTTPPVLDSDGSF | | TKSFNRGEC | |
| | FLYSRLTVDKSRWQEGNVFSCSVMHEAL | | | |
| | HNHYTQKSLSLSLGK | | | |
| 91 | EVQLLESGGGLVQPGGSLRLSCAASGFTF | 1805 | DIQMTQSPSSLSASVG | 1893 |
| | SNSDMNWVRQAPGKGLEWVSAISGSGGS | | DRVTITCRASQSISSYL | |
| | TYYADSVKGRFTISRDNSKNTLYLQMNS | | NWYQQKPGKAPKLLI | |
| | LRAEDTAVYYCARGREDDYGDYVFDYW | | YGASTLHSGVPSRFSG | |
| | GQGTLVTVSSSTKGPSVFPLAPCSRSTSES | | SGSGTDFTLTISSLQPE | |
| | TAALGCLVKDYFPEPVTVSWNSGALTSG | | DFATYYCQQSYRIPYT | |
| | VHTFPAVLQSSGLYSLSSVVTVPSSSLGTK | | FGQGTKLEIKRTVAAP | |
| | TYTCNVDHKPSNTKVDKRVSKYGPPCPS | | SVFIFPPSDEQLKSGTA | |
| | CPPEFLGGPSVFLFPPKPKDTLMISRTPEV | | SVVCLLNNFYPREAK | |
| | TCVVVDVSQEDPEVQFNWYVDGVEVHN | | VQWKVDNALQSGNS | |
| | AKTKPREEQFNSTYRVVSVLTVLHQDWL | | QESVTEQDSKDSTYSL | |
| | NGKEYKCKVSNKGLPSSIEKTISKAKQPR | | SSTLTLSKADYEKHKV | |
| | EPQVYTLPPSQEEMTKNQVSLTCLVKGFY | | YACEVTHQGLSSPVT | |
| | PSDIAVEWESNGQPENNYKTTPPVLDSDG | | KSFNRGEC | |
| | SFFLYSRLTVDKSRWQEGNVFSCSVMHE | | | |
| | ALHNHYTQKSLSLSLGK | | | |
| 92 | QVQLVQSGAEVKKPGASVKVSCKASGGT | 1806 | EIVMTQSPATLSVSPG | 1894 |
| | FSSYAISWVRQAPGQGLEWMGWINPNSG | | ERATLSCRASQNINSD | |
| | NTGYAQKFQGRVTMTRDTSTSTVYMELS | | LAWYQQKPGQAPRLL | |
| | SLRSEDTAVYYCAREHGDMDVWGQGTT | | IYGASTRATGIPARFSG | |
| | VTVSSSTKGPSVFPLAPCSRSTSESTAALG | | SGSGTEFTLTISSLQSE | |
| | CLVKDYFPEPVTVSWNSGALTSGVHTFP | | DFAVYYCQQYDSLPF | |
| | AVLQSSGLYSLSSVVTVPSSSLGTKTYTC | | TFGPGTKVDIKRTVAA | |
| | NVDHKPSNTKVDKRVSKYGPPCPSCPPEF | | PSVFIFPPSDEQLKSGT | |
| | LGGPSVFLFPPKPKDTLMISRTPEVTCVVV | | ASVVCLLNNFYPREA | |
| | DVSQEDPEVQFNWYVDGVEVHNAKTKP | | KVQWKVDNALQSGN | |
| | REEQFNSTYRVVSVLTVLHQDWLNGKEY | | SQESVTEQDSKDSTYS | |
| | KCKVSNKGLPSSIEKTISKAKQPREPQVYT | | LSSTLTLSKADYEKHK | |
| | LPPSQEEMTKNQVSLTCLVKGFYPSDIAV | | VYACEVTHQGLSSPV | |
| | EWESNGQPENNYKTTPPVLDSDGSFFLYS | | TKSFNRGEC | |
| | RLTVDKSRWQEGNVFSCSVMHEALHNH | | | |
| | YTQKSLSLSLGK | | | |
| 93 | QVQLVQSGAEVKKPGASVKVSCKASGNT | 1807 | DIVMTQSPLSLPVTPG | 1895 |
| | FTSYGISWVRQAPGQGLEWMGWINPNSG | | EPASISCRSSQSLLHSN | |
| | GTKYAQKFQGRVTMTRDTSTSTVYMELS | | GYNYLDWYLQKPGQS | |
| | SLRSEDTAVYYCARESWFGELYYGMDV | | PQLLIYLGSDRASGVP | |
| | WGKGTTVTVSSSTKGPSVFPLAPCSRSTS | | DRFSGSGSGTDFTLKIS | |
| | ESTAALGCLVKDYFPEPVTVSWNSGALTS | | RVEAEDVGVYYCMQ | |
| | GVHTFPAVLQSSGLYSLSSVVTVPSSSLGT | | GLQTPITFGQGTRLEIK | |
| | KTYTCNVDHKPSNTKVDKRVSKYGPPCP | | RTVAAPSVFIFPPSDEQ | |
| | SCPPEFLGGPSVFLFPPKPKDTLMISRTPE | | LKSGTASVVCLLNNF | |
| | VTCVVVDVSQEDPEVQFNWYVDGVEVH | | YPREAKVQWKVDNA | |
| | NAKTKPREEQFNSTYRVVSVLTVLHQDW | | LQSGNSQESVTEQDSK | |
| | LNGKEYKCKVSNKGLPSSIEKTISKAKQP | | DSTYSLSSTLTLSKAD | |
| | REPQVYTLPPSQEEMTKNQVSLTCLVKGF | | YEKHKVYACEVTHQG | |
| | YPSDIAVEWESNGQPENNYKTTPPVLDSD | | LSSPVTKSFNRGEC | |
| | GSFFLYSRLTVDKSRWQEGNVFSCSVMH | | | |
| | EALHNHYTQKSLSLSLGK | | | |
| 94 | QVQLVQSGAEVKKPGASVKVSCKASGYT | 1808 | DIQMTQSPSSLSASVG | 1896 |
| | FTAYYTHWVRQAPGQGLEWMGWMNPN | | DRVTITCRASQSISSYL | |
| | SGHTSYAQKFQGRVTMTRDTSTSTVYME | | NWYQQKPGKAPKLLI | |
| | LSSLRSEDTAVYYCAREAYDSFDYWGQG | | YEASTLETGVPSRFSG | |
| | TLVTVSSSTKGPSVFPLAPCSRSTSESTAA | | SGSGTDFTLTISSLQPE | |
| | LGCLVKDYFPEPVTVSWNSGALTSGVHT | | DFATYYCQQANSFPFT | |
| | FPAVLQSSGLYSLSSVVTVPSSSLGTKTYT | | FGPGTKVDIKRTVAAP | |
| | CNVDHKPSNTKVDKRVSKYGPPCPSCPPE | | SVFIFPPSDEQLKSGTA | |
| | FLGGPSVFLFPPKPKDTLMISRTPEVTCVV | | SVVCLLNNFYPREAK | |
| | VDVSQEDPEVQFNWYVDGVEVHNAKTK | | VQWKVDNALQSGNS | |
| | PREEQFNSTYRVVSVLTVLHQDWLNGKE | | QESVTEQDSKDSTYSL | |
| | YKCKVSNKGLPSSIEKTISKAKQPREPQV | | SSTLTLSKADYEKHKV | |
| | YTLPPSQEEMTKNQVSLTCLVKGFYPSDI | | YACEVTHQGLSSPVT | |
| | AVEWESNGQPENNYKTTPPVLDSDGSFFL | | KSFNRGEC | |
| | YSRLTVDKSRWQEGNVFSCSVMHEALHN | | | |
| | HYTQKSLSLSLGK | | | |
| 95 | QVQLVQSGAEVKKPGASVKVSCKASGYT | 1809 | DIQMTQSPSSLSASVG | 1897 |
| | FTDYYMHWVRQAPGQGLEWMGWINPNS | | DRVTITCRASRGINNW | |
| | GGTNYAQKFQGRVTMTRDTSTSTVYMEL | | LTWYQQKPGKAPKLL | |
| | SSLRSEDTAVYYCARDSRIAVAASSFDYW | | IYGASSLQSGVPSRFS | |
| | GQGTLVTVSSSTKGPSVFPLAPCSRSTSES | | GSGSGTDFTLTISSLQP | |
| | TAALGCLVKDYFPEPVTVSWNSGALTSG | | EDFATYYCQQSYRIPY | |
| | VHTFPAVLQSSGLYSLSSVVTVPSSSLGTK | | TFGQGTKLEIKRTVAA | |
| | TYTCNVDHKPSNTKVDKRVSKYGPPCPS | | PSVFIFPPSDEQLKSGT | |
| | CPPEFLGGPSVFLFPPKPKDTLMISRTPEV | | ASVVCLLNNFYPREA | |
| | TCVVVDVSQEDPEVQFNWYVDGVEVHN | | KVQWKVDNALQSGN | |
| | AKTKPREEQFNSTYRVVSVLTVLHQDWL | | SQESVTEQDSKDSTYS | |
| | NGKEYKCKVSNKGLPSSIEKTISKAKQPR | | LSSTLTLSKADYEKHK | |
| | EPQVYTLPPSQEEMTKNQVSLTCLVKGFY | | VYACEVTHQGLSSPV | |
| | PSDIAVEWESNGQPENNYKTTPPVLDSDG | | TKSFNRGEC | |
| | SFFLYSRLTVDKSRWQEGNVFSCSVMHE | | | |
| | ALHNHYTQKSLSLSLGK | | | |
| 96 | EVQLLESGGGLVQPGGSLRLSCAASGFTF | 1810 | DIQMTQSPSSLSASVG | 1898 |
| | SSYAMSWVRQAPGKGLEWVSDISGSGSG | | DRVTITCRASQSVSSF | |
| | TYYADAVKGRFTISRDNSKNTLYLQMNS | | LNWYQQKPGKAPKLL | |
| | LRAEDTAVYYCARPGSDGEFDYWGQGT | | IYAASSLQSGVPSRFS | |
| | LVTVSSSTKGPSVFPLAPCSRSTSESTAAL | | GSGSGTDFTLTISSLQP | |
| | GCLVKDYFPEPVTVSWNSGALTSGVHTF | | EDFATYYCQQSYTTPL | |
| | PAVLQSSGLYSLSSVVTVPSSSLGTKTYTC | | TFGQGTKVEIKRTVAA | |
| | NVDHKPSNTKVDKRVSKYGPPCPSCPPEF | | PSVFIFPPSDEQLKSGT | |
| | LGGPSVFLFPPKPKDTLMISRTPEVTCVVV | | ASVVCLLNNFYPREA | |
| | DVSQEDPEVQFNWYVDGVEVHNAKTKP | | KVQWKVDNALQSGN | |
| | REEQFNSTYRVVSVLTVLHQDWLNGKEY | | SQESVTEQDSKDSTYS | |
| | KCKVSNKGLPSSIEKTISKAKQPREPQVYT | | LSSTLTLSKADYEKHK | |
| | LPPSQEEMTKNQVSLTCLVKGFYPSDIAV | | VYACEVTHQGLSSPV | |
| | EWESNGQPENNYKTTPPVLDSDGSFFLYS | | TKSFNRGEC | |
| | RLTVDKSRWQEGNVFSCSVMHEALHNH | | | |
| | YTQKSLSLSLGK | | | |
| 97 | QVQLVQSGAEVKKPGSSVKVSCKASGGT | 1811 | DIQMTQSPSSLSASVG | 1899 |
| | FSSDAINWVRQAPGQGLEWMGGFDPEDG | | DRVTITCRSSRNISHW | |
| | ETIYAQKFQGRVTITADESTSTAYMELSSL | | LAWYQQKPGKAPKLL | |
| | RSEDTAVYYCARGPSGYDFEFDYWGQGT | | IYKASSLESGVPSRFSG | |
| | LVTVSSSTKGPSVFPLAPCSRSTSESTAAL | | SGSGTDFTLTISSLQPE | |
| | GCLVKDYFPEPVTVSWNSGALTSGVHTF | | DFATYYCQQAISFPLT | |
| | PAVLQSSGLYSLSSVVTVPSSSLGTKTYTC | | FGGGTKVEIKRTVAAP | |
| | NVDHKPSNTKVDKRVSKYGPPCPSCPPEF | | SVFIFPPSDEQLKSGTA | |
| | LGGPSVFLFPPKPKDTLMISRTPEVTCVVV | | SVVCLLNNFYPREAK | |
| | DVSQEDPEVQFNWYVDGVEVHNAKTKP | | VQWKVDNALQSGNS | |
| | REEQFNSTYRVVSVLTVLHQDWLNGKEY | | QESVTEQDSKDSTYSL | |
| | KCKVSNKGLPSSIEKTISKAKQPREPQVYT | | SSTLTLSKADYEKHKV | |
| | LPPSQEEMTKNQVSLTCLVKGFYPSDIAV | | YACEVTHQGLSSPVT | |
| | EWESNGQPENNYKTTPPVLDSDGSFFLYS | | KSFNRGEC | |
| | RLTVDKSRWQEGNVFSCSVMHEALHNH | | | |
| | YTQKSLSLSLGK | | | |
| 98 | QVQLVQSGAEVKKPGASVKVSCKASGDT | 1812 | DIVMTQSPDSLAVSLG | 1900 |
| | FTTYAISWVRQAPGQGLEWMGWINPNSG | | ERATINCKSSQSVLHS | |
| | VATYANKFQGRVTMTRDTSTSTVYMELS | | SKNKNYLAWYQQKP | |
| | SLRSEDTAVYYCAREGIVGATDAFDIWG | | GQPPKLLIYWASTRES | |
| | QGTMVTVSSSTKGPSVFPLAPCSRSTSEST | | GVPDRFSGSGSGTDFT | |
| | AALGCLVKDYFPEPVTVSWNSGALTSGV | | LTISSLQAEDVAVYYC | |
| | HTFPAVLQSSGLYSLSSVVTVPSSSLGTKT | | QQYFTTPPTFGPGTKV | |
| | YTCNVDHKPSNTKVDKRVSKYGPPCPSC | | DIKRTVAAPSVFIFPPS | |
| | PPEFLGGPSVFLFPPKPKDTLMISRTPEVT | | DEQLKSGTASVVCLL | |
| | CVVVDVSQEDPEVQFNWYVDGVEVHNA | | NNFYPREAKVQWKV | |
| | KTKPREEQFNSTYRVVSVLTVLHQDWLN | | DNALQSGNSQESVTE | |
| | GKEYKCKVSNKGLPSSIEKTISKAKQPREP | | QDSKDSTYSLSSTLTL | |
| | QVYTLPPSQEEMTKNQVSLTCLVKGFYPS | | SKADYEKHKVYACEV | |
| | DIAVEWESNGQPENNYKTTPPVLDSDGSF | | THQGLSSPVTKSFNRG | |
| | FLYSRLTVDKSRWQEGNVFSCSVMHEAL | | EC | |
| | HNHYTQKSLSLSLGK | | | |
| 99 | QVQLVQSGAEVKKPGASVKVSCKASGDT | 1813 | DIQMTQSPSSLSASVG | 1901 |
| | FTNHYMHWVRQAPGQGLEWMGWINPNS | | DRVTITCRASQSLGSW | |
| | GGTNYAQKFQGRVTMTRDTSTSTVYMEL | | LAWYQQKPGKAPKLL | |
| | SSLRSEDTAVYYCARDLVPAAVGGYFDY | | IYAASSLQSGVPSRFS | |
| | WGQGTLVTVSSSTKGPSVFPLAPCSRSTS | | GSGSGTDFTLTISSLQP | |
| | ESTAALGCLVKDYFPEPVTVSWNSGALTS | | EDFATYYCQQANSFPL | |
| | GVHTFPAVLQSSGLYSLSSVVTVPSSSLGT | | TFGQGTKVEIKRTVAA | |
| | KTYTCNVDHKPSNTKVDKRVSKYGPPCP | | PSVFIFPPSDEQLKSGT | |
| | SCPPEFLGGPSVFLFPPKPKDTLMISRTPE | | ASVVCLLNNFYPREA | |
| | VTCVVVDVSQEDPEVQFNWYVDGVEVH | | KVQWKVDNALQSGN | |
| | NAKTKPREEQFNSTYRVVSVLTVLHQDW | | SQESVTEQDSKDSTYS | |
| | LNGKEYKCKVSNKGLPSSIEKTISKAKQP | | LSSTLTLSKADYEKHK | |
| | REPQVYTLPPSQEEMTKNQVSLTCLVKGF | | VYACEVTHQGLSSPV | |
| | YPSDIAVEWESNGQPENNYKTTPPVLDSD | | TKSFNRGEC | |
| | GSFFLYSRLTVDKSRWQEGNVFSCSVMH | | | |
| | EALHNHYTQKSLSLSLGK | | | |
| 100 | EVQLLESGGGLVQPGGSLRLSCAASGFTF | 1814 | DIQMTQSPSSLSASVG | 1902 |
| | SSHWMSWVRQAPGKGLEWVSAISGSGGS | | DRVTITCQASQDIDNY | |
| | TYYADSVKGRFTISRDNSKNTLYLQMNS | | LNWYQQKPGKAPKLL | |
| | LRAEDTAVYYCARDDNSGSQADWGQGT | | IYDASNLETGVPSRFS | |
| | LVTVSSSTKGPSVFPLAPCSRSTSESTAAL | | GSGSGTDFTLTISSLQP | |
| | GCLVKDYFPEPVTVSWNSGALTSGVHTF | | EDFATYYCQQSYSTPL | |
| | PAVLQSSGLYSLSSVVTVPSSSLGTKTYTC | | TFGGGTKLEIKRTVAA | |
| | NVDHKPSNTKVDKRVSKYGPPCPSCPPEF | | PSVFIFPPSDEQLKSGT | |
| | LGGPSVFLFPPKPKDTLMISRTPEVTCVVV | | ASVVCLLNNFYPREA | |
| | DVSQEDPEVQFNWYVDGVEVHNAKTKP | | KVQWKVDNALQSGN | |
| | REEQFNSTYRVVSVLTVLHQDWLNGKEY | | SQESVTEQDSKDSTYS | |
| | KCKVSNKGLPSSIEKTISKAKQPREPQVYT | | LSSTLTLSKADYEKHK | |
| | LPPSQEEMTKNQVSLTCLVKGFYPSDIAV | | VYACEVTHQGLSSPV | |
| | EWESNGQPENNYKTTPPVLDSDGSFFLYS | | TKSFNRGEC | |
| | RLTVDKSRWQEGNVFSCSVMHEALHNH | | | |
| | YTQKSLSLSLGK | | | |
| 101 | QVQLVQSGAEVKKPGASVKVSCKASGYS | 1815 | DIQMTQSPSSLSASVG | 1903 |
| | FTGYYMHWVRQAPGQGLEWMGWINPNS | | DRVTITCRASQGIRNW | |
| | GGTYFAQNFQGRVTMTRDTSTSTVYMEL | | LAWYQQKPGKAPKLL | |
| | SSLRSEDTAVYYCVKDRGDRVVTSYLDY | | IYAASSLQSGVPSRFS | |
| | WGQGTLVTVSSSTKGPSVFPLAPCSRSTS | | GSGSGTDFTLTISSLQP | |
| | ESTAALGCLVKDYFPEPVTVSWNSGALTS | | EDFATYYCQQSYRTP | |
| | GVHTFPAVLQSSGLYSLSSVVTVPSSSLGT | | YTFGQGTKLEIKRTVA | |
| | KTYTCNVDHKPSNTKVDKRVSKYGPPCP | | APSVFIFPPSDEQLKSG | |
| | SCPPEFLGGPSVFLFPPKPKDTLMISRTPE | | TASVVCLLNNFYPREA | |
| | VTCVVVDVSQEDPEVQFNWYVDGVEVH | | KVQWKVDNALQSGN | |
| | NAKTKPREEQFNSTYRVVSVLTVLHQDW | | SQESVTEQDSKDSTYS | |
| | LNGKEYKCKVSNKGLPSSIEKTISKAKQP | | LSSTLTLSKADYEKHK | |
| | REPQVYTLPPSQEEMTKNQVSLTCLVKGF | | VYACEVTHQGLSSPV | |
| | YPSDIAVEWESNGQPENNYKTTPPVLDSD | | TKSFNRGEC | |
| | GSFFLYSRLTVDKSRWQEGNVFSCSVMH | | | |
| | EALHNHYTQKSLSLSLGK | | | |
| 102 | QVQLVQSGAEVKKPGASVKVSCKASGYT | 1816 | DIVMTQSPDSLAVSLG | 1904 |
| | FTGYYMHWVRQAPGQGLEWMGIINPSG | | ERATINCKSSQSVLYS | |
| | GSTSYAQKFQGRVTMTRDTSTSTVYMEL | | SNNKNYLAWYQQKP | |
| | SSLRSEDTAVYYCARAAPYYYDSSGYYS | | GQPPKLLIYWASTRES | |
| | GGYYFDYWGQGTLVTVSSSTKGPSVFPL | | GVPDRFSGSGSGTDFT | |
| | APCSRSTSESTAALGCLVKDYFPEPVTVS | | LTISSLQAEDVAVYYC | |
| | WNSGALTSGVHTFPAVLQSSGLYSLSSVV | | QQYYTTPLTFGQGTK | |
| | TVPSSSLGTKTYTCNVDHKPSNTKVDKR | | LEIKRTVAAPSVFIFPP | |
| | VSKYGPPCPSCPPEFLGGPSVFLFPPKPKD | | SDEQLKSGTASVVCLL | |
| | TLMISRTPEVTCVVVDVSQEDPEVQFNW | | NNFYPREAKVQWKV | |
| | YVDGVEVHNAKTKPREEQFNSTYRVVSV | | DNALQSGNSQESVTE | |
| | LTVLHQDWLNGKEYKCKVSNKGLPSSIE | | QDSKDSTYSLSSTLTL | |
| | KTISKAKQPREPQVYTLPPSQEEMTKNQV | | SKADYEKHKVYACEV | |
| | SLTCLVKGFYPSDIAVEWESNGQPENNYK | | THQGLSSPVTKSFNRG | |
| | TTPPVLDSDGSFFLYSRLTVDKSRWQEGN | | EC | |
| | VFSCSVMHEALHNHYTQKSLSLSLGK | | | |
| 103 | EVQLLESGGGLVQPGGSLRLSCAASGFTF | 1817 | DIVMTQSPLSLPVTPG | 1905 |
| | SIYEIHWVRQAPGKGLEWVSAISGSGGST | | EPASISCRSSQSLLHSN | |
| | YYADSVKGRFTISRDNSKNTLYLQMNSL | | GYNYLDWYLQKPGQS | |
| | RAEDTAVYYCARSYCGGDCWDYYYYYG | | PQLLIYLASNRASGVP | |
| | MDVWGQGTTVTVSSSTKGPSVFPLAPCS | | DRFSGSGSGTDFTLKIS | |
| | RSTSESTAALGCLVKDYFPEPVTVSWNSG | | RVEAEDVGVYYCKQT | |
| | ALTSGVHTFPAVLQSSGLYSLSSVVTVPSS | | SHIPLTFGQGTK VEIKR | |
| | SLGTKTYTCNVDHKPSNTKVDKRVSKYG | | TVAAPSVFIFPPSDEQL | |
| | PPCPSCPPEFLGGPSVFLFPPKPKDTLMISR | | KSGTASVVCLLNNFYP | |
| | TPEVTCVVVDVSQEDPEVQFNWYVDGVE | | REAKVQWKVDNALQ | |
| | VHNAKTKPREEQFNSTYRVVSVLTVLHQ | | SGNSQESVTEQDSKDS | |
| | DWLNGKEYKCKVSNKGLPSSIEKTISKAK | | TYSLSSTLTLSKADYE | |
| | QPREPQVYTLPPSQEEMTKNQVSLTCLVK | | KHKVYACEVTHQGLS | |
| | GFYPSDIAVEWESNGQPENNYKTTPPVLD | | SPVTKSFNRGEC | |
| | SDGSFFLYSRLTVDKSRWQEGNVFSCSV | | | |
| | MHEALHNHYTQKSLSLSLGK | | | |
| 104 | EVQLVESGGGLVKPGGSLRLSCAASGFTF | 1818 | DIVMTQSPLSLPVTPG | 1906 |
| | SDNSMNWVRQAPGKGLEWVSYISSSGSTI | | EPASISCRSSQSLLHSN | |
| | YYADSVKGRFTISRDDSKNTLYLQMNSL | | GYNYLDWYLQKPGQS | |
| | KTEDTAVYYCARGRASSWPNWFDPWGQ | | PQLLIYSASNLQSGVP | |
| | GTLVTVSSSTKGPSVFPLAPCSRSTSESTA | | DRFSGSGSGTDFTLKIS | |
| | ALGCLVKDYFPEPVTVSWNSGALTSGVH | | RVEAEDVGVYYCMQ | |
| | TFPAVLQSSGLYSLSSVVTVPSSSLGTKTY | | ALQTPPTFGQGTKLEI | |
| | TCNVDHKPSNTKVDKRVSKYGPPCPSCPP | | KRTVAAPSVFIFPPSDE | |
| | EFLGGPSVFLFPPKPKDTLMISRTPEVTCV | | QLKSGTASVVCLLNN | |
| | VVDVSQEDPEVQFNWYVDGVEVHNAKT | | FYPREAKVQWKVDN | |
| | KPREEQFNSTYRVVSVLTVLHQDWLNGK | | ALQSGNSQESVTEQDS | |
| | EYKCKVSNKGLPSSIEKTISKAKQPREPQV | | KDSTYSLSSTLTLSKA | |
| | YTLPPSQEEMTKNQVSLTCLVKGFYPSDI | | DYEKHKVYACEVTHQ | |
| | AVEWESNGQPENNYKTTPPVLDSDGSFFL | | GLSSPVTKSFNRGEC | |
| | YSRLTVDKSRWQEGNVFSCSVMHEALHN | | | |
| | HYTQKSLSLSLGK | | | |
| 105 | EVQLLESGGGLVQPGGSLRLSCAASGFTF | 1819 | DIQMTQSPSSLSASVG | 1907 |
| | SSYAMSWVRQAPGKGLEWVSGISYDSDK | | DRVTITCRASQGISNN | |
| | IGYADAVKGRFTISRDNSKNTLYLQMNSL | | LNWYQQKPGKAPKLL | |
| | RAEDTAVYYCAREWEGFDYWGQGTLVT | | IYESSTLETGVPSRFSG | |
| | VSSSTKGPSVFPLAPCSRSTSESTAALGCL | | SGSGTDFTLTISSLQPE | |
| | VKDYFPEPVTVSWNSGALTSGVHTFPAV | | DFATYYCQQSYSAPLT | |
| | LQSSGLYSLSSVVTVPSSSLGTKTYTCNV | | FGGGTKVEIKRTVAAP | |
| | DHKPSNTKVDKRVSKYGPPCPSCPPEFLG | | SVFIFPPSDEQLKSGTA | |
| | GPSVFLFPPKPKDTLMISRTPEVTCVVVD | | SVVCLLNNFYPREAK | |
| | VSQEDPEVQFNWYVDGVEVHNAKTKPR | | VQWKVDNALQSGNS | |
| | EEQFNSTYRVVSVLTVLHQDWLNGKEYK | | QESVTEQDSKDSTYSL | |
| | CKVSNKGLPSSIEKTISKAKQPREPQVYTL | | SSTLTLSKADYEKHKV | |
| | PPSQEEMTKNQVSLTCLVKGFYPSDIAVE | | YACEVTHQGLSSPVT | |
| | WESNGQPENNYKTTPPVLDSDGSFFLYSR | | KSFNRGEC | |
| | LTVDKSRWQEGNVFSCSVMHEALHNHY | | | |
| | TQKSLSLSLGK | | | |
| 106 | QVQLVQSGAEVKKPGASVKVSCKASGYT | 1820 | DIVMTQSPLSLPVTPG | 1908 |
| | FTDHYMHWVRQAPGQGLEWMGWINPNS | | EPASISCRSSQSLLHSN | |
| | GGTNYAQKFQGRVTMTRDTSTSTVYMEL | | GYNYLDWYLQKPGQS | |
| | SSLRSEDTAVYYCAKDKFGDEGSGWYGD | | PQLLIYLGSNRASGVP | |
| | FQHWGQGTLVTVSSSTKGPSVFPLAPCSR | | DRFSGSGSGTDFTLKIS | |
| | STSESTAALGCLVKDYFPEPVTVSWNSGA | | RVEAEDVGVYYCMQ | |
| | LTSGVHTFPAVLQSSGLYSLSSVVTVPSSS | | TLRTPLTFGGGTKVEI | |
| | LGTKTYTCNVDHKPSNTKVDKRVSKYGP | | KRTVAAPSVFIFPPSDE | |
| | PCPSCPPEFLGGPSVFLFPPKPKDTLMISRT | | QLKSGTASVVCLLNN | |
| | PEVTCVVVDVSQEDPEVQFNWYVDGVE | | FYPREAKVQWKVDN | |
| | VHNAKTKPREEQFNSTYRVVSVLTVLHQ | | ALQSGNSQESVTEQDS | |
| | DWLNGKEYKCKVSNKGLPSSIEKTISKAK | | KDSTYSLSSTLTLSKA | |
| | QPREPQVYTLPPSQEEMTKNQVSLTCLVK | | DYEKHKVYACEVTHQ | |
| | GFYPSDIAVEWESNGQPENNYKTTPPVLD | | GLSSPVTKSFNRGEC | |
| | SDGSFFLYSRLTVDKSRWQEGNVFSCSV | | | |
| | MHEALHNHYTQKSLSLSLGK | | | |
| 107 | EVQLLESGGGLVQPGGSLRLSCAASGFTF | 1821 | DIQMTQSPSSLSASVG | 1909 |
| | SSYWMHWVRQAPGKGLEWVSGFSGSAR | | DRVTITCRASQNIGPW | |
| | TYYADSVKGRFTISRDNSKNTLYLQMNS | | LAWYQQKPGKAPKLL | |
| | LRAEDTAVYYCAREWSGFDYWGQGTLV | | IYDAKDLHPGVPSRFS | |
| | TVSSSTKGPSVFPLAPCSRSTSESTAALGC | | GSGSGTDFTLTISSLQP | |
| | LVKDYFPEPVTVSWNSGALTSGVHTFPA | | EDFATYYCQQANTFP | |
| | VLQSSGLYSLSSVVTVPSSSLGTKTYTCN | | MTFGQGTRLEIKRTVA | |
| | VDHKPSNTKVDKRVSKYGPPCPSCPPEFL | | APSVFIFPPSDEQLKSG | |
| | GGPSVFLFPPKPKDTLMISRTPEVTCVVV | | TASVVCLLNNFYPREA | |
| | DVSQEDPEVQFNWYVDGVEVHNAKTKP | | KVQWKVDNALQSGN | |
| | REEQFNSTYRVVSVLTVLHQDWLNGKEY | | SQESVTEQDSKDSTYS | |
| | KCKVSNKGLPSSIEKTISKAKQPREPQVYT | | LSSTLTLSKADYEKHK | |
| | LPPSQEEMTKNQVSLTCLVKGFYPSDIAV | | VYACEVTHQGLSSPV | |
| | EWESNGQPENNYKTTPPVLDSDGSFFLYS | | TKSFNRGEC | |
| | RLTVDKSRWQEGNVFSCSVMHEALHNH | | | |
| | YTQKSLSLSLGK | | | |
| 108 | QVQLVQSGAEVKKPGASVKVSCKASGY | 1822 | DIQMTQSPSSLSASVG | 1910 |
| | MFTGYYIHWVRQAPGQGLEWMGWINPN | | DRVTITCRASQSIDRW | |
| | SGGTNYAQKFQGRVTMTRDTSTSTVYME | | LAWYQQKPGKAPKLL | |
| | LSSLRSEDTAVYYCAKDRFGSGNYGYMD | | IYGASSLQSGVPSRFS | |
| | VWGKGTTVTVSSSTKGPSVFPLAPCSRST | | GSGSGTDFTLTISSLQP | |
| | SESTAALGCLVKDYFPEPVTVSWNSGALT | | EDFATYYCQQSYSTP | |
| | SGVHTFPAVLQSSGLYSLSSVVTVPSSSLG | | WTFGQGTRLEIKRTV | |
| | TKTYTCNVDHKPSNTKVDKRVSKYGPPC | | AAPSVFIFPPSDEQLKS | |
| | PSCPPEFLGGPSVFLFPPKPKDTLMISRTPE | | GTASVVCLLNNFYPRE | |
| | VTCVVVDVSQEDPEVQFNWYVDGVEVH | | AKVQWKVDNALQSG | |
| | NAKTKPREEQFNSTYRVVSVLTVLHQDW | | NSQESVTEQDSKDSTY | |
| | LNGKEYKCKVSNKGLPSSIEKTISKAKQP | | SLSSTLTLSKADYEKH | |
| | REPQVYTLPPSQEEMTKNQVSLTCLVKGF | | KVYACEVTHQGLSSP | |
| | YPSDIAVEWESNGQPENNYKTTPPVLDSD | | VTKSFNRGEC | |
| | GSFFLYSRLTVDKSRWQEGNVFSCSVMH | | | |
| | EALHNHYTQKSLSLSLGK | | | |
| 109 | EVQLLESGGGLVQPGGSLRLSCAASGFTF | 1823 | DIQMTQSPSSLSASVG | 1911 |
| | SSYAMSWVRQAPGKGLEWVSAISGSGGS | | DRVTITCQASQDISNN | |
| | TYYADSVKGRFTISRDNSKNTLYLQMNS | | LNWYQQKPGKAPKLL | |
| | LRAEDTAVYYCARELSHDYGGNSDFDY | | IYAASGLQSGVPSRFS | |
| | WGQGTLVTVSSSTKGPSVFPLAPCSRSTS | | GSGSGTDFTLTISSLQP | |
| | ESTAALGCLVKDYFPEPVTVSWNSGALTS | | EDFATYYCQQANSFPL | |
| | GVHTFPAVLQSSGLYSLSSVVTVPSSSLGT | | TFGGGTKVEIKRTVAA | |
| | KTYTCNVDHKPSNTKVDKRVSKYGPPCP | | PSVFIFPPSDEQLKSGT | |
| | SCPPEFLGGPSVFLFPPKPKDTLMISRTPE | | ASVVCLLNNFYPREA | |
| | VTCVVVDVSQEDPEVQFNWYVDGVEVH | | KVQWKVDNALQSGN | |
| | NAKTKPREEQFNSTYRVVSVLTVLHQDW | | SQESVTEQDSKDSTYS | |
| | LNGKEYKCKVSNKGLPSSIEKTISKAKQP | | LSSTLTLSKADYEKHK | |
| | REPQVYTLPPSQEEMTKNQVSLTCLVKGF | | VYACEVTHQGLSSPV | |
| | YPSDIAVEWESNGQPENNYKTTPPVLDSD | | TKSFNRGEC | |
| | GSFFLYSRLTVDKSRWQEGNVFSCSVMH | | | |
| | EALHNHYTQKSLSLSLGK | | | |
| 110 | QVQLVQSGAEVKKPGASVKVSCKASGYT | 1824 | DIQMTQSPSSLSASVG | 1912 |
| | FTDYYIHWVRQAPGQGLEWMGWINPNS | | DRVTITCRASRSIRTW | |
| | GGTNYAQEFQGRVTMTRDTSTSTVYMEL | | LAWYQQKPGKAPKLL | |
| | SSLRSEDTAVYYCARDHRIAVAGSYFDY | | IYAASSLQTGVPSRFS | |
| | WGQGTLVTVSSSTKGPSVFPLAPCSRSTS | | GSGSGTDFTLTISSLQP | |
| | ESTAALGCLVKDYFPEPVTVSWNSGALTS | | EDFATYYCQQSYSTPY | |
| | GVHTFPAVLQSSGLYSLSSVVTVPSSSLGT | | TFGQGTKLEIKRTVAA | |
| | KTYTCNVDHKPSNTKVDKRVSKYGPPCP | | PSVFIFPPSDEQLKSGT | |
| | SCPPEFLGGPSVFLFPPKPKDTLMISRTPE | | ASVVCLLNNFYPREA | |
| | VTCVVVDVSQEDPEVQFNWYVDGVEVH | | KVQWKVDNALQSGN | |
| | NAKTKPREEQFNSTYRVVSVLTVLHQDW | | SQESVTEQDSKDSTYS | |
| | LNGKEYKCKVSNKGLPSSIEKTISKAKQP | | LSSTLTLSKADYEKHK | |
| | REPQVYTLPPSQEEMTKNQVSLTCLVKGF | | VYACEVTHQGLSSPV | |
| | YPSDIAVEWESNGQPENNYKTTPPVLDSD | | TKSFNRGEC | |
| | GSFFLYSRLTVDKSRWQEGNVFSCSVMH | | | |
| | EALHNHYTQKSLSLSLGK | | | |
| 111 | QVQLVQSGAEVKKPGASVKVSCKASGYP | 1825 | DIQMTQSPSSLSASVG | 1913 |
| | FTAHYIHWVRQAPGQGLEWMGWINPNS | | DRVTITCRASQGINNW | |
| | GGTNYAQKFQGRVTMTRDTSTSTVYMEL | | LAWYQQKPGKAPKLL | |
| | SSLRSEDTAVYYCARDVEMATIGAYWYF | | IYDASNLETGVPSRFS | |
| | DLWGRGTLVTVSSSTKGPSVFPLAPCSRS | | GSGSGTDFTLTISSLQP | |
| | TSESTAALGCLVKDYFPEPVTVSWNSGAL | | EDFATYYCQQANSFPP | |
| | TSGVHTFPAVLQSSGLYSLSSVVTVPSSSL | | TFGQGTKLEIKRTVAA | |
| | GTKTYTCNVDHKPSNTKVDKRVSKYGPP | | PSVFIFPPSDEQLKSGT | |
| | CPSCPPEFLGGPSVFLFPPKPKDTLMISRTP | | ASVVCLLNNFYPREA | |
| | EVTCVVVDVSQEDPEVQFNWYVDGVEV | | KVQWKVDNALQSGN | |
| | HNAKTKPREEQFNSTYRVVSVLTVLHQD | | SQESVTEQDSKDSTYS | |
| | WLNGKEYKCKVSNKGLPSSIEKTISKAKQ | | LSSTLTLSKADYEKHK | |
| | PREPQVYTLPPSQEEMTKNQVSLTCLVKG | | VYACEVTHQGLSSPV | |
| | FYPSDIAVEWESNGQPENNYKTTPPVLDS | | TKSFNRGEC | |
| | DGSFFLYSRLTVDKSRWQEGNVFSCSVM | | | |
| | HEALHNHYTQKSLSLSLGK | | | |
| 112 | QVQLVQSGAEVKKPGSSVKVSCKASGYS | 1826 | DIVMTQSPLSLPVTPG | 1914 |
| | FTSYGISWVRQAPGQGLEWLGWISAYNG | | EPASISCRSSQSLLHSN | |
| | NTNYGQSLQGRVTITADESTSTAYMELSS | | GYNYLDWYLQKPGQS | |
| | LRSEDTAVYYCARARGAGTFFDYWGQG | | PQLLIYDATNLPTGVP | |
| | TLVTVSSSTKGPSVFPLAPCSRSTSESTAA | | DRFSGSGSGTDFTLKIS | |
| | LGCLVKDYFPEPVTVSWNSGALTSGVHT | | RVEAEDVGVYYCMQ | |
| | FPAVLQSSGLYSLSSVVTVPSSSLGTKTYT | | ALQTPFTFGQGTKLEI | |
| | CNVDHKPSNTKVDKRVSKYGPPCPSCPPE | | KRTVAAPSVFIFPPSDE | |
| | FLGGPSVFLFPPKPKDTLMISRTPEVTCVV | | QLKSGTASVVCLLNN | |
| | VDVSQEDPEVQFNWYVDGVEVHNAKTK | | FYPREAKVQWKVDN | |
| | PREEQFNSTYRVVSVLTVLHQDWLNGKE | | ALQSGNSQESVTEQDS | |
| | YKCKVSNKGLPSSIEKTISKAKQPREPQV | | KDSTYSLSSTLTLSKA | |
| | YTLPPSQEEMTKNQVSLTCLVKGFYPSDI | | DYEKHKVYACEVTHQ | |
| | AVEWESNGQPENNYKTTPPVLDSDGSFFL | | GLSSPVTKSFNRGEC | |
| | YSRLTVDKSRWQEGNVFSCSVMHEALHN | | | |
| | HYTQKSLSLSLGK | | | |
| 113 | QVQLVQSGAEVKKPGASVKVSCKASGYT | 1827 | DIQMTQSPSSLSASVG | 1915 |
| | FTGYYMHWVRQAPGQGLEWMGRINPNG | | DRVTITCRASQSINDW | |
| | GSTTYAQKFQGRVTMTRDTSTSTVYMEL | | LAWYQQKPGKAPKLL | |
| | SSLRSEDTAVYYCARDDFYYYYLDFWGK | | IYAASNLQSGVPSRFS | |
| | GTTVTVSSSTKGPSVFPLAPCSRSTSESTA | | GSGSGTDFTLTISSLQP | |
| | ALGCLVKDYFPEPVTVSWNSGALTSGVH | | EDFATYYCQQGYSTPP | |
| | TFPAVLQSSGLYSLSSVVTVPSSSLGTKTY | | TFGQGTKVEIKRTVAA | |
| | TCNVDHKPSNTKVDKRVSKYGPPCPSCPP | | PSVFIFPPSDEQLKSGT | |
| | EFLGGPSVFLFPPKPKDTLMISRTPEVTCV | | ASVVCLLNNFYPREA | |
| | VVDVSQEDPEVQFNWYVDGVEVHNAKT | | KVQWKVDNALQSGN | |
| | KPREEQFNSTYRVVSVLTVLHQDWLNGK | | SQESVTEQDSKDSTYS | |
| | EYKCKVSNKGLPSSIEKTISKAKQPREPQV | | LSSTLTLSKADYEKHK | |
| | YTLPPSQEEMTKNQVSLTCLVKGFYPSDI | | VYACEVTHQGLSSPV | |
| | AVEWESNGQPENNYKTTPPVLDSDGSFFL | | TKSFNRGEC | |
| | YSRLTVDKSRWQEGNVFSCSVMHEALHN | | | |
| | HYTQKSLSLSLGK | | | |
| 114 | QVQLVQSGAEVKKPGASVKVSCKASGYT | 1828 | DIQMTQSPSSLSASVG | 1916 |
| | FTENEMHWVRQAPGQGLEWMGWMNPN | | DRVTITCQASQDIRNY | |
| | SGNTGYAQKFQGRVTMTRDTSTSTVYME | | LNWYQQKPGKAPKLL | |
| | LSSLRSEDTAVYYCAREGGDWPYYYMD | | IYAASSLQSGVPSRFS | |
| | VWGKGTTVTVSSSTKGPSVFPLAPCSRST | | GSGSGTDFTLTISSLQP | |
| | SESTAALGCLVKDYFPEPVTVSWNSGALT | | EDFATYYCQQTSSTPL | |
| | SGVHTFPAVLQSSGLYSLSSVVTVPSSSLG | | TFGPGTKVDIKRTVAA | |
| | TKTYTCNVDHKPSNTKVDKRVSKYGPPC | | PSVFIFPPSDEQLKSGT | |
| | PSCPPEFLGGPSVFLFPPKPKDTLMISRTPE | | ASVVCLLNNFYPREA | |
| | VTCVVVDVSQEDPEVQFNWYVDGVEVH | | KVQWKVDNALQSGN | |
| | NAKTKPREEQFNSTYRVVSVLTVLHQDW | | SQESVTEQDSKDSTYS | |
| | LNGKEYKCKVSNKGLPSSIEKTISKAKQP | | LSSTLTLSKADYEKHK | |
| | REPQVYTLPPSQEEMTKNQVSLTCLVKGF | | VYACEVTHQGLSSPV | |
| | YPSDIAVEWESNGQPENNYKTTPPVLDSD | | TKSFNRGEC | |
| | GSFFLYSRLTVDKSRWQEGNVFSCSVMH | | | |
| | EALHNHYTQKSLSLSLGK | | | |
| 115 | QVQLVQSGAEVKKPGASVKVSCKASGYT | 1829 | DIQMTQSPSSLSASVG | 1917 |
| | LTGYYMHWVRQAPGQGLEWMGWMNPS | | DRVTITCRASQDIRNN | |
| | SGNTGYAQQFQGRVTMTRDTSTSTVYME | | LGWYQQKPGKAPKLL | |
| | LSSLRSEDTAVYYCARASSDRYYYDGVW | | IYGASSLQSGVPSRFS | |
| | YFDLWGRGTLVTVSSSTKGPSVFPLAPCS | | GSGSGTDFTLTISSLQP | |
| | RSTSESTAALGCLVKDYFPEPVTVSWNSG | | EDFATYYCQQTYSSPP | |
| | ALTSGVHTFPAVLQSSGLYSLSSVVTVPSS | | TFGQGTKLEIKRTVAA | |
| | SLGTKTYTCNVDHKPSNTKVDKRVSKYG | | PSVFIFPPSDEQLKSGT | |
| | PPCPSCPPEFLGGPSVFLFPPKPKDTLMISR | | ASVVCLLNNFYPREA | |
| | TPEVTCVVVDVSQEDPEVQFNWYVDGVE | | KVQWKVDNALQSGN | |
| | VHNAKTKPREEQFNSTYRVVSVLTVLHQ | | SQESVTEQDSKDSTYS | |
| | DWLNGKEYKCKVSNKGLPSSIEKTISKAK | | LSSTLTLSKADYEKHK | |
| | QPREPQVYTLPPSQEEMTKNQVSLTCLVK | | VYACEVTHQGLSSPV | |
| | GFYPSDIAVEWESNGQPENNYKTTPPVLD | | TKSFNRGEC | |
| | SDGSFFLYSRLTVDKSRWQEGNVFSCSV | | | |
| | MHEALHNHYTQKSLSLSLGK | | | |
| 116 | EVQLLESGGGLVQPGGSLRLSCAASGFTF | 1830 | DIQMTQSPSSLSASVG | 1918 |
| | STYAMHWVRQAPGKGLEWVSAISGSGGS | | DRVTITCRASQGIDNY | |
| | TYYADSVKGRFTISRDNSKNTLYLQMNS | | LAWYQQKPGKAPKLL | |
| | LRAEDTAVYYCARDGYGDYPFDYWGQG | | IYQASTLESGVPSRFSG | |
| | TLVTVSSSTKGPSVFPLAPCSRSTSESTAA | | SGSGTDFTLTISSLQPE | |
| | LGCLVKDYFPEPVTVSWNSGALTSGVHT | | DFATYYCQQSYSIPWT | |
| | FPAVLQSSGLYSLSSVVTVPSSSLGTKTYT | | FGQGTKVEIKRTVAAP | |
| | CNVDHKPSNTKVDKRVSKYGPPCPSCPPE | | SVFIFPPSDEQLKSGTA | |
| | FLGGPSVFLFPPKPKDTLMISRTPEVTCVV | | SVVCLLNNFYPREAK | |
| | VDVSQEDPEVQFNWYVDGVEVHNAKTK | | VQWKVDNALQSGNS | |
| | PREEQFNSTYRVVSVLTVLHQDWLNGKE | | QESVTEQDSKDSTYSL | |
| | YKCKVSNKGLPSSIEKTISKAKQPREPQV | | SSTLTLSKADYEKHKV | |
| | YTLPPSQEEMTKNQVSLTCLVKGFYPSDI | | YACEVTHQGLSSPVT | |
| | AVEWESNGQPENNYKTTPPVLDSDGSFFL | | KSFNRGEC | |
| | YSRLTVDKSRWQEGNVFSCSVMHEALHN | | | |
| | HYTQKSLSLSLGK | | | |
| 117 | QVQLVQSGAEVKKPGASVKVSCKASGYT | 1831 | DIQMTQSPSSLSASVG | 1919 |
| | FTGYYLHWVRQAPGQGLEWMGVINVRR | | DRVTITCRASQSISRW | |
| | GSTRYAQNFQGRVTMTRDTSTSTVYMEL | | LAWYQQKPGKAPKLL | |
| | SSLRSEDTAVYYCARVSGSYYQPWGQGT | | IYDASNLETGVPSRFS | |
| | LVTVSSSTKGPSVFPLAPCSRSTSESTAAL | | GSGSGTDFTLTISSLQP | |
| | GCLVKDYFPEPVTVSWNSGALTSGVHTF | | EDFATYYCQQGNSFPP | |
| | PAVLQSSGLYSLSSVVTVPSSSLGTKTYTC | | IFGGGTKVEIKRTVAA | |
| | NVDHKPSNTKVDKRVSKYGPPCPSCPPEF | | PSVFIFPPSDEQLKSGT | |
| | LGGPSVFLFPPKPKDTLMISRTPEVTCVVV | | ASVVCLLNNFYPREA | |
| | DVSQEDPEVQFNWYVDGVEVHNAKTKP | | KVQWKVDNALQSGN | |
| | REEQFNSTYRVVSVLTVLHQDWLNGKEY | | SQESVTEQDSKDSTYS | |
| | KCKVSNKGLPSSIEKTISKAKQPREPQVYT | | LSSTLTLSKADYEKHK | |
| | LPPSQEEMTKNQVSLTCLVKGFYPSDIAV | | VYACEVTHQGLSSPV | |
| | EWESNGQPENNYKTTPPVLDSDGSFFLYS | | TKSFNRGEC | |
| | RLTVDKSRWQEGNVFSCSVMHEALHNH | | | |
| | YTQKSLSLSLGK | | | |
| 118 | QVQLVQSGAEVKKPGASVKVSCKASGYT | 1832 | DIQMTQSPSSLSASVG | 1920 |
| | FSNYYMHWVRQAPGQGLEWMGWMNPD | | DRVTITCRASQSISSW | |
| | SGTTGYAQKFQGRVTMTRDTSTSTVYME | | LAWYQQKPGKAPKLL | |
| | LSSLRSEDTAVYYCVRDGTMVQGIFDYW | | IYGASSLQSGVPSRFS | |
| | GQGTLVTVSSSTKGPSVFPLAPCSRSTSES | | GSGSGTDFTLTISSLQP | |
| | TAALGCLVKDYFPEPVTVSWNSGALTSG | | EDFATYYCQQTYRTP | |
| | VHTFPAVLQSSGLYSLSSVVTVPSSSLGTK | | LTFGPGTKVDIKRTVA | |
| | TYTCNVDHKPSNTKVDKRVSKYGPPCPS | | APSVFIFPPSDEQLKSG | |
| | CPPEFLGGPSVFLFPPKPKDTLMISRTPEV | | TASVVCLLNNFYPREA | |
| | TCVVVDVSQEDPEVQFNWYVDGVEVHN | | KVQWKVDNALQSGN | |
| | AKTKPREEQFNSTYRVVSVLTVLHQDWL | | SQESVTEQDSKDSTYS | |
| | NGKEYKCKVSNKGLPSSIEKTISKAKQPR | | LSSTLTLSKADYEKHK | |
| | EPQVYTLPPSQEEMTKNQVSLTCLVKGFY | | VYACEVTHQGLSSPV | |
| | PSDIAVEWESNGQPENNYKTTPPVLDSDG | | TKSFNRGEC | |
| | SFFLYSRLTVDKSRWQEGNVFSCSVMHE | | | |
| | ALHNHYTQKSLSLSLGK | | | |
| 119 | QVQLVQSGAEVKKPGSSVKVSCKASGGT | 1833 | DIQMTQSPSSLSASVG | 1921 |
| | FSTYAITWVRQAPGQGLEWMGGIIPIVGR | | DRVTITCRASQGIGND | |
| | ANYAQKFQGRVTITADESTSTAYMELSSL | | LGWYQQKPGKAPKLL | |
| | RSEDTAVYYCARSGGHDLDYWGQGTLV | | IYGASSVQSGVPSRFS | |
| | TVSSSTKGPSVFPLAPCSRSTSESTAALGC | | GSGSGTDFTLTISSLQP | |
| | LVKDYFPEPVTVSWNSGALTSGVHTFPA | | EDFATYYCQQSYSTPI | |
| | VLQSSGLYSLSSVVTVPSSSLGTKTYTCN | | TFGQGTRLEIKRTVAA | |
| | VDHKPSNTKVDKRVSKYGPPCPSCPPEFL | | PSVFIFPPSDEQLKSGT | |
| | GGPSVFLFPPKPKDTLMISRTPEVTCVVV | | ASVVCLLNNFYPREA | |
| | DVSQEDPEVQFNWYVDGVEVHNAKTKP | | KVQWKVDNALQSGN | |
| | REEQFNSTYRVVSVLTVLHQDWLNGKEY | | SQESVTEQDSKDSTYS | |
| | KCKVSNKGLPSSIEKTISKAKQPREPQVYT | | LSSTLTLSKADYEKHK | |
| | LPPSQEEMTKNQVSLTCLVKGFYPSDIAV | | VYACEVTHQGLSSPV | |
| | EWESNGQPENNYKTTPPVLDSDGSFFLYS | | TKSFNRGEC | |
| | RLTVDKSRWQEGNVFSCSVMHEALHNH | | | |
| | YTQKSLSLSLGK | | | |
| 120 | EVQLLESGGGLVQPGGSLRLSCAASGFTF | 1834 | EIVMTQSPATLSVSPG | 1922 |
| | SSYGMHWVRQAPGKGLEWVSSISGSGDT | | ERATLSCRASQSVSSS | |
| | TYYADSVKGRFTISRDNSKNTLYLQMNS | | YLAWYQQKPGQAPRL | |
| | LRAEDTAVYYCARDNPYGDYGGSFDYW | | LIYATSTRATGIPARFS | |
| | GQGTLVTVSSSTKGPSVFPLAPCSRSTSES | | GSGSGTEFTLTISSLQS | |
| | TAALGCLVKDYFPEPVTVSWNSGALTSG | | EDFAVYYCQQYGSLP | |
| | VHTFPAVLQSSGLYSLSSVVTVPSSSLGTK | | LTFGQGTKVEIKRTVA | |
| | TYTCNVDHKPSNTKVDKRVSKYGPPCPS | | APSVFIFPPSDEQLKSG | |
| | CPPEFLGGPSVFLFPPKPKDTLMISRTPEV | | TASVVCLLNNFYPREA | |
| | TCVVVDVSQEDPEVQFNWYVDGVEVHN | | KVQWKVDNALQSGN | |
| | AKTKPREEQFNSTYRVVSVLTVLHQDWL | | SQESVTEQDSKDSTYS | |
| | NGKEYKCKVSNKGLPSSIEKTISKAKQPR | | LSSTLTLSKADYEKHK | |
| | EPQVYTLPPSQEEMTKNQVSLTCLVKGFY | | VYACEVTHQGLSSPV | |
| | PSDIAVEWESNGQPENNYKTTPPVLDSDG | | TKSFNRGEC | |
| | SFFLYSRLTVDKSRWQEGNVFSCSVMHE | | | |
| | ALHNHYTQKSLSLSLGK | | | |
| 121 | QVQLVQSGAEVKKPGASVKVSCKASGYT | 1835 | DIQMTQSPSSLSASVG | 1923 |
| | FTSYYMHWVRQAPGQGLEWMGIIDPSGG | | DRVTITCRASQGISNN | |
| | STNYAQKFQGRVTMTRDTSTSTVYMELS | | LNWYQQKPGKAPKLL | |
| | SLRSEDTAVYYCARDYYGSGSYYGLDY | | IYDASNLETGVPSRFS | |
| | WGRGTLVTVSSSTKGPSVFPLAPCSRSTS | | GSGSGTDFTLTISSLQP | |
| | ESTAALGCLVKDYFPEPVTVSWNSGALTS | | EDFATYYCQQANSFPL | |
| | GVHTFPAVLQSSGLYSLSSVVTVPSSSLGT | | TFGPGTKVDIKRTVAA | |
| | KTYTCNVDHKPSNTKVDKRVSKYGPPCP | | PSVFIFPPSDEQLKSGT | |
| | SCPPEFLGGPSVFLFPPKPKDTLMISRTPE | | ASVVCLLNNFYPREA | |
| | VTCVVVDVSQEDPEVQFNWYVDGVEVH | | KVQWKVDNALQSGN | |
| | NAKTKPREEQFNSTYRVVSVLTVLHQDW | | SQESVTEQDSKDSTYS | |
| | LNGKEYKCKVSNKGLPSSIEKTISKAKQP | | LSSTLTLSKADYEKHK | |
| | REPQVYTLPPSQEEMTKNQVSLTCLVKGF | | VYACEVTHQGLSSPV | |
| | YPSDIAVEWESNGQPENNYKTTPPVLDSD | | TKSFNRGEC | |
| | GSFFLYSRLTVDKSRWQEGNVFSCSVMH | | | |
| | EALHNHYTQKSLSLSLGK | | | |
| 122 | QVQLVQSGAEVKKPGASVKVSCKASGYT | 1836 | DIQMTQSPSSLSASVG | 1924 |
| | FTDYYMHWVRQAPGQGLEWMGIINPSG | | DRVTITCRASQGIRND | |
| | GSTRYAQKFQGRVTMTRDTSTSTVYMEL | | LAWYQQKPGKAPKLL | |
| | SSLRSEDTAVYYCARVDGRRWLQSDYW | | IYAASTLQNGVPSRFS | |
| | GQGTLVTVSSSTKGPSVFPLAPCSRSTSES | | GSGSGTDFTLTISSLQP | |
| | TAALGCLVKDYFPEPVTVSWNSGALTSG | | EDFATYYCQQSYSTP | |
| | VHTFPAVLQSSGLYSLSSVVTVPSSSLGTK | | WTFGQGTKVEIKRTV | |
| | TYTCNVDHKPSNTKVDKRVSKYGPPCPS | | AAPSVFIFPPSDEQLKS | |
| | CPPEFLGGPSVFLFPPKPKDTLMISRTPEV | | GTASVVCLLNNFYPRE | |
| | TCVVVDVSQEDPEVQFNWYVDGVEVHN | | AKVQWKVDNALQSG | |
| | AKTKPREEQFNSTYRVVSVLTVLHQDWL | | NSQESVTEQDSKDSTY | |
| | NGKEYKCKVSNKGLPSSIEKTISKAKQPR | | SLSSTLTLSKADYEKH | |
| | EPQVYTLPPSQEEMTKNQVSLTCLVKGFY | | KVYACEVTHQGLSSP | |
| | PSDIAVEWESNGQPENNYKTTPPVLDSDG | | VTKSFNRGEC | |
| | SFFLYSRLTVDKSRWQEGNVFSCSVMHE | | | |
| | ALHNHYTQKSLSLSLGK | | | |
| 123 | QVQLVQSGAEVKKPGASVKVSCKASGYT | 1837 | DIQMTQSPSSLSASVG | 1925 |
| | FTDYYMHWVRQAPGQGLEWMGIINPSG | | DRVTITCRASQGIRND | |
| | GSTRYAQKFQGRVTMTRDTSTSTVYMEL | | LAWYQQKPGKAPKLL | |
| | SSLRSEDTAVYYCARVDGRRWLRSDYW | | IYAASTLQNGVPSRFS | |
| | GQGTLVTVSSSTKGPSVFPLAPCSRSTSES | | GSGSGTDFTLTISSLQP | |
| | TAALGCLVKDYFPEPVTVSWNSGALTSG | | EDFATYYCQQSYSTP | |
| | VHTFPAVLQSSGLYSLSSVVTVPSSSLGTK | | WTFGQGTKVEIKRTV | |
| | TYTCNVDHKPSNTKVDKRVSKYGPPCPS | | AAPSVFIFPPSDEQLKS | |
| | CPPEFLGGPSVFLFPPKPKDTLMISRTPEV | | GTASVVCLLNNFYPRE | |
| | TCVVVDVSQEDPEVQFNWYVDGVEVHN | | AKVQWKVDNALQSG | |
| | AKTKPREEQFNSTYRVVSVLTVLHQDWL | | NSQESVTEQDSKDSTY | |
| | NGKEYKCKVSNKGLPSSIEKTISKAKQPR | | SLSSTLTLSKADYEKH | |
| | EPQVYTLPPSQEEMTKNQVSLTCLVKGFY | | KVYACEVTHQGLSSP | |
| | PSDIAVEWESNGQPENNYKTTPPVLDSDG | | VTKSFNRGEC | |
| | SFFLYSRLTVDKSRWQEGNVFSCSVMHE | | | |
| | ALHNHYTQKSLSLSLGK | | | |
| 124 | QVQLVQSGAEVKKPGASVKVSCKASGGT | 1838 | DIQMTQSPSSLSASVG | 1926 |
| | FSSYAISWVRQAPGQGLEWLGIISPSGRSA | | DRVTITCQASQGINNY | |
| | GYGRKFQGRVTMTRDTSTSTVYMELSSL | | LNWYQQKPGKAPKLL | |
| | RSEDTAVYYCARTDYGGHKWYFDLWGR | | IYAASTLQRGVPSRFS | |
| | GTLVTVSSSTKGPSVFPLAPCSRSTSESTA | | GSGSGTDFTLTISSLQP | |
| | ALGCLVKDYFPEPVTVSWNSGALTSGVH | | EDFATYYCQQSYQTP | |
| | TFPAVLQSSGLYSLSSVVTVPSSSLGTKTY | | LTFGGGTKVEIKRTVA | |
| | TCNVDHKPSNTKVDKRVSKYGPPCPSCPP | | APSVFIFPPSDEQLKSG | |
| | EFLGGPSVFLFPPKPKDTLMISRTPEVTCV | | TASVVCLLNNFYPREA | |
| | VVDVSQEDPEVQFNWYVDGVEVHNAKT | | KVQWKVDNALQSGN | |
| | KPREEQFNSTYRVVSVLTVLHQDWLNGK | | SQESVTEQDSKDSTYS | |
| | EYKCKVSNKGLPSSIEKTISKAKQPREPQV | | LSSTLTLSKADYEKHK | |
| | YTLPPSQEEMTKNQVSLTCLVKGFYPSDI | | VYACEVTHQGLSSPV | |
| | AVEWESNGQPENNYKTTPPVLDSDGSFFL | | TKSFNRGEC | |
| | YSRLTVDKSRWQEGNVFSCSVMHEALHN | | | |
| | HYTQKSLSLSLGK | | | |
| 125 | QVQLVQSGAEVKKPGASVKVSCKASGYT | 1839 | DIQMTQSPSSLSASVG | 1927 |
| | FTGYYLHWVRQAPGQGLEWMGVISPSG | | DRVTITCRASQSISSYL | |
| | GGTSYAQKFQGRVTMTRDTSTSTVYMEL | | NWYQQKPGKAPKLLI | |
| | SSLRSEDTAVYYCARAGFGEGVFRHWGQ | | YAASSLQSGVPSRFSG | |
| | GTLVTVSSSTKGPSVFPLAPCSRSTSESTA | | SGSGTDFTLTISSLQPE | |
| | ALGCLVKDYFPEPVTVSWNSGALTSGVH | | DFATYYCQQSYSTPLT | |
| | TFPAVLQSSGLYSLSSVVTVPSSSLGTKTY | | FGGGTKVEIKRTVAAP | |
| | TCNVDHKPSNTKVDKRVSKYGPPCPSCPP | | SVFIFPPSDEQLKSGTA | |
| | EFLGGPSVFLFPPKPKDTLMISRTPEVTCV | | SVVCLLNNFYPREAK | |
| | VVDVSQEDPEVQFNWYVDGVEVHNAKT | | VQWKVDNALQSGNS | |
| | KPREEQFNSTYRVVSVLTVLHQDWLNGK | | QESVTEQDSKDSTYSL | |
| | EYKCKVSNKGLPSSIEKTISKAKQPREPQV | | SSTLTLSKADYEKHKV | |
| | YTLPPSQEEMTKNQVSLTCLVKGFYPSDI | | YACEVTHQGLSSPVT | |
| | AVEWESNGQPENNYKTTPPVLDSDGSFFL | | KSFNRGEC | |
| | YSRLTVDKSRWQEGNVFSCSVMHEALHN | | | |
| | HYTQKSLSLSLGK | | | |
| 126 | QVQLVQSGAEVKKPGASVKVSCKASGYS | 1840 | DIQMTQSPSSLSASVG | 1928 |
| | FTSHAISWVRQAPGQGLEWMGWIKPNSG | | DRVTITCRASQGISNY | |
| | DTKYAQKFQGRVTMTRDTSTSTVYMELS | | LAWYQQKPGKAPKLL | |
| | SLRSEDTAVYYCARGSDDYYGSYYFDY | | IYTASTLQSGVPSRFSG | |
| | WGQGTLVTVSSSTKGPSVFPLAPCSRSTS | | SGSGTDFTLTISSLQPE | |
| | ESTAALGCLVKDYFPEPVTVSWNSGALTS | | DFATYYCQQSYSTPLT | |
| | GVHTFPAVLQSSGLYSLSSVVTVPSSSLGT | | FGGGTKVEIKRTVAAP | |
| | KTYTCNVDHKPSNTKVDKRVSKYGPPCP | | SVFIFPPSDEQLKSGTA | |
| | SCPPEFLGGPSVFLFPPKPKDTLMISRTPE | | SVVCLLNNFYPREAK | |
| | VTCVVVDVSQEDPEVQFNWYVDGVEVH | | VQWKVDNALQSGNS | |
| | NAKTKPREEQFNSTYRVVSVLTVLHQDW | | QESVTEQDSKDSTYSL | |
| | LNGKEYKCKVSNKGLPSSIEKTISKAKQP | | SSTLTLSKADYEKHKV | |
| | REPQVYTLPPSQEEMTKNQVSLTCLVKGF | | YACEVTHQGLSSPVT | |
| | YPSDIAVEWESNGQPENNYKTTPPVLDSD | | KSFNRGEC | |
| | GSFFLYSRLTVDKSRWQEGNVFSCSVMH | | | |
| | EALHNHYTQKSLSLSLGK | | | |
| 127 | EVQLLESGGGLVQPGGSLRLSCAASGFTF | 1841 | DIQMTQSPSSLSASVG | 1929 |
| | RNYGMGWVRQAPGKGLEWVSAISGSGG | | DRVTITCRASQGISND | |
| | STYYADSVKGRFTISRDNSKNTLYLQMNS | | LAWYQQKPGKAPKLL | |
| | LRAEDTAVYYCARVKFYGMDVWGQGTT | | IYGASNLETGVPSRFS | |
| | VTVSSSTKGPSVFPLAPCSRSTSESTAALG | | GSGSGTDFTLTISSLQP | |
| | CLVKDYFPEPVTVSWNSGALTSGVHTFP | | EDFATYYCQQANSFPF | |
| | AVLQSSGLYSLSSVVTVPSSSLGTKTYTC | | TFGPGTKVDIKRTVAA | |
| | NVDHKPSNTKVDKRVSKYGPPCPSCPPEF | | PSVFIFPPSDEQLKSGT | |
| | LGGPSVFLFPPKPKDTLMISRTPEVTCVVV | | ASVVCLLNNFYPREA | |
| | DVSQEDPEVQFNWYVDGVEVHNAKTKP | | KVQWKVDNALQSGN | |
| | REEQFNSTYRVVSVLTVLHQDWLNGKEY | | SQESVTEQDSKDSTYS | |
| | KCKVSNKGLPSSIEKTISKAKQPREPQVYT | | LSSTLTLSKADYEKHK | |
| | LPPSQEEMTKNQVSLTCLVKGFYPSDIAV | | VYACEVTHQGLSSPV | |
| | EWESNGQPENNYKTTPPVLDSDGSFFLYS | | TKSFNRGEC | |
| | RLTVDKSRWQEGNVFSCSVMHEALHNH | | | |
| | YTQKSLSLSLGK | | | |
| 128 | QVQLVQSGAEVKKPGASVKVSCKASGYT | 1842 | DIQMTQSPSSLSASVG | 1930 |
| | FTDYHMHWVRQAPGQGLEWMGWMSPN | | DRVTITCRVSQGISSYL | |
| | SGNTGYAQNFQGRVTMTRDTSTSTVYME | | NWYQQKPGKAPKLLI | |
| | LSSLRSEDTAVYYCARADYYGSDYVKFD | | YEASTLESGVPSRFSG | |
| | YWGQGTLVTVSSSTKGPSVFPLAPCSRST | | SGSGTDFTLTISSLQPE | |
| | SESTAALGCLVKDYFPEPVTVSWNSGALT | | DFATYYCQQGYSTPPT | |
| | SGVHTFPAVLQSSGLYSLSSVVTVPSSSLG | | FGQGTKVEIKRTVAAP | |
| | TKTYTCNVDHKPSNTKVDKRVSKYGPPC | | SVFIFPPSDEQLKSGTA | |
| | PSCPPEFLGGPSVFLFPPKPKDTLMISRTPE | | SVVCLLNNFYPREAK | |
| | VTCVVVDVSQEDPEVQFNWYVDGVEVH | | VQWKVDNALQSGNS | |
| | NAKTKPREEQFNSTYRVVSVLTVLHQDW | | QESVTEQDSKDSTYSL | |
| | LNGKEYKCKVSNKGLPSSIEKTISKAKQP | | SSTLTLSKADYEKHKV | |
| | REPQVYTLPPSQEEMTKNQVSLTCLVKGF | | YACEVTHQGLSSPVT | |
| | YPSDIAVEWESNGQPENNYKTTPPVLDSD | | KSFNRGEC | |
| | GSFFLYSRLTVDKSRWQEGNVFSCSVMH | | | |
| | EALHNHYTQKSLSLSLGK | | | |
| 129 | QVQLVQSGAEVKKPGASVKVSCKASGYT | 1843 | DIVMTQSPLSLPVTPG | 1931 |
| | FPNYGISWVRQAPGQGLEWMGWINPNSG | | EPASISCRSSQSLLQSN | |
| | GTKYAQRFQGRVTMTRDTSTSTVYMELS | | GYNYLDWYLQKPGQS | |
| | SLRSEDTAVYYCARDRDILTGYYHFDYW | | PQLLIYLGSNRASGVP | |
| | GQGTLVTVSSSTKGPSVFPLAPCSRSTSES | | DRFSGSGSGTDFTLKIS | |
| | TAALGCLVKDYFPEPVTVSWNSGALTSG | | RVEAEDVGVYYCMQS | |
| | VHTFPAVLQSSGLYSLSSVVTVPSSSLGTK | | THWPLTFGQGTRLEIK | |
| | TYTCNVDHKPSNTKVDKRVSKYGPPCPS | | RTVAAPSVFIFPPSDEQ | |
| | CPPEFLGGPSVFLFPPKPKDTLMISRTPEV | | LKSGTASVVCLLNNF | |
| | TCVVVDVSQEDPEVQFNWYVDGVEVHN | | YPREAKVQWKVDNA | |
| | AKTKPREEQFNSTYRVVSVLTVLHQDWL | | LQSGNSQESVTEQDSK | |
| | NGKEYKCKVSNKGLPSSIEKTISKAKQPR | | DSTYSLSSTLTLSKAD | |
| | EPQVYTLPPSQEEMTKNQVSLTCLVKGFY | | YEKHKVYACEVTHQG | |
| | PSDIAVEWESNGQPENNYKTTPPVLDSDG | | LSSPVTKSFNRGEC | |
| | SFFLYSRLTVDKSRWQEGNVFSCSVMHE | | | |
| | ALHNHYTQKSLSLSLGK | | | |
| 130 | QVQLVQSGAEVKKPGASVKVSCKASGYT | 1844 | DIQMTQSPSSLSASVG | 1932 |
| | FTDYFMHWVRQAPGQGLEWMGWINPNS | | DRVTITCRASQGISNN | |
| | GNTGYAQKFQGRVTMTRDTSTSTVYMEL | | LNWYQQKPGKAPKLL | |
| | SSLRSEDTAVYYCARLNDYGDYGGPATL | | IYAASSLQSGVPSRFS | |
| | DYWGQGTLVTVSSSTKGPSVFPLAPCSRS | | GSGSGTDFTLTISSLQP | |
| | TSESTAALGCLVKDYFPEPVTVSWNSGAL | | EDFATYYCQQSYSTPP | |
| | TSGVHTFPAVLQSSGLYSLSSVVTVPSSSL | | TFGQGTKLEIKRTVAA | |
| | GTKTYTCNVDHKPSNTKVDKRVSKYGPP | | PSVFIFPPSDEQLKSGT | |
| | CPSCPPEFLGGPSVFLFPPKPKDTLMISRTP | | ASVVCLLNNFYPREA | |
| | EVTCVVVDVSQEDPEVQFNWYVDGVEV | | KVQWKVDNALQSGN | |
| | HNAKTKPREEQFNSTYRVVSVLTVLHQD | | SQESVTEQDSKDSTYS | |
| | WLNGKEYKCKVSNKGLPSSIEKTISKAKQ | | LSSTLTLSKADYEKHK | |
| | PREPQVYTLPPSQEEMTKNQVSLTCLVKG | | VYACEVTHQGLSSPV | |
| | FYPSDIAVEWESNGQPENNYKTTPPVLDS | | TKSFNRGEC | |
| | DGSFFLYSRLTVDKSRWQEGNVFSCSVM | | | |
| | HEALHNHYTQKSLSLSLGK | | | |
| 131 | QVQLVQSGAEVKKPGASVKVSCKASGYT | 1845 | DIQMTQSPSSLSASVG | 1933 |
| | FTNYYMHWVRQAPGQGLEWLGWISPYS | | DRVTITCRASQSISTYL | |
| | GDTKYAQTLQGRVTMTRDTSTSTVYMEL | | NWYQQKPGKAPKLLI | |
| | SSLRSEDTAVYYCARESMDRLDYWGQGT | | YDASNLETGVPSRFSG | |
| | LVTVSSSTKGPSVFPLAPCSRSTSESTAAL | | SGSGTDFTLTISSLQPE | |
| | GCLVKDYFPEPVTVSWNSGALTSGVHTF | | DFATYYCQQSYSTPVL | |
| | PAVLQSSGLYSLSSVVTVPSSSLGTKTYTC | | TFGGGTKVEIKRTVAA | |
| | NVDHKPSNTKVDKRVSKYGPPCPSCPPEF | | PSVFIFPPSDEQLKSGT | |
| | LGGPSVFLFPPKPKDTLMISRTPEVTCVVV | | ASVVCLLNNFYPREA | |
| | DVSQEDPEVQFNWYVDGVEVHNAKTKP | | KVQWKVDNALQSGN | |
| | REEQFNSTYRVVSVLTVLHQDWLNGKEY | | SQESVTEQDSKDSTYS | |
| | KCKVSNKGLPSSIEKTISKAKQPREPQVYT | | LSSTLTLSKADYEKHK | |
| | LPPSQEEMTKNQVSLTCLVKGFYPSDIAV | | VYACEVTHQGLSSPV | |
| | EWESNGQPENNYKTTPPVLDSDGSFFLYS | | TKSFNRGEC | |
| | RLTVDKSRWQEGNVFSCSVMHEALHNH | | | |
| | YTQKSLSLSLGK | | | |
| 132 | EVQLLESGGGLVQPGGSLRLSCAASGFTF | 1846 | DIVMTQSPLSLPVTPG | 1934 |
| | SSYAMHWVRQAPGKGLEWVADISGSGG | | EPASISCRSSQSLLHSN | |
| | LTYYADSVKGRFTISRDNSKNTLYLQMN | | GYNYLDWYLQKPGQS | |
| | SLRAEDTAVYYCAREGDQYSSSSFFDYW | | PQLLIYLGSNRASGVP | |
| | GQGTLVTVSSSTKGPSVFPLAPCSRSTSES | | DRFSGSGSGTDFTLKIS | |
| | TAALGCLVKDYFPEPVTVSWNSGALTSG | | RVEAEDVGVYYCMQ | |
| | VHTFPAVLQSSGLYSLSSVVTVPSSSLGTK | | ALQPPPTFGQGTRLEI | |
| | TYTCNVDHKPSNTKVDKRVSKYGPPCPS | | KRTVAAPSVFIFPPSDE | |
| | CPPEFLGGPSVFLFPPKPKDTLMISRTPEV | | QLKSGTASVVCLLNN | |
| | TCVVVDVSQEDPEVQFNWYVDGVEVHN | | FYPREAKVQWKVDN | |
| | AKTKPREEQFNSTYRVVSVLTVLHQDWL | | ALQSGNSQESVTEQDS | |
| | NGKEYKCKVSNKGLPSSIEKTISKAKQPR | | KDSTYSLSSTLTLSKA | |
| | EPQVYTLPPSQEEMTKNQVSLTCLVKGFY | | DYEKHKVYACEVTHQ | |
| | PSDIAVEWESNGQPENNYKTTPPVLDSDG | | GLSSPVTKSFNRGEC | |
| | SFFLYSRLTVDKSRWQEGNVFSCSVMHE | | | |
| | ALHNHYTQKSLSLSLGK | | | |
| 133 | EVQLVESGGGLVKPGGSLRLSCAASGFTF | 1847 | DIQMTQSPSSLSASVG | 1935 |
| | DEFGMNWVRQAPGKGLEWISYISGDSGY | | DRVTITCQASQDIDIYL | |
| | TNCADSVKGRFTISRDDSKNTLYLQMNSL | | NWYQQKPGKAPKLLI | |
| | KTEDTAVYYCAAGYGGYYFDYWGQGTL | | YAASTLESGVPSRFSG | |
| | VTVSSSTKGPSVFPLAPCSRSTSESTAALG | | SGSGTDFTLTISSLQPE | |
| | CLVKDYFPEPVTVSWNSGALTSGVHTFP | | DFATYYCQQSYSTPPT | |
| | AVLQSSGLYSLSSVVTVPSSSLGTKTYTC | | FGGGTKVEIKRTVAAP | |
| | NVDHKPSNTKVDKRVSKYGPPCPSCPPEF | | SVFIFPPSDEQLKSGTA | |
| | LGGPSVFLFPPKPKDTLMISRTPEVTCVVV | | SVVCLLNNFYPREAK | |
| | DVSQEDPEVQFNWYVDGVEVHNAKTKP | | VQWKVDNALQSGNS | |
| | REEQFNSTYRVVSVLTVLHQDWLNGKEY | | QESVTEQDSKDSTYSL | |
| | KCKVSNKGLPSSIEKTISKAKQPREPQVYT | | SSTLTLSKADYEKHKV | |
| | LPPSQEEMTKNQVSLTCLVKGFYPSDIAV | | YACEVTHQGLSSPVT | |
| | EWESNGQPENNYKTTPPVLDSDGSFFLYS | | KSFNRGEC | |
| | RLTVDKSRWQEGNVFSCSVMHEALHNH | | | |
| | YTQKSLSLSLGK | | | |
| 134 | QVQLVQSGAEVKKPGASVKVSCKASGYT | 1848 | DIQMTQSPSSLSASVG | 1936 |
| | FTSYYMHWVRQAPGQGLEWMGMINPSA | | DRVTITCRASQSISTYL | |
| | GSTSYAQKFQGRVTMTRDTSTSTVYMEL | | NWYQQKPGKAPKLLI | |
| | SSLRSEDTAVYYCASVDSSGWYAPFDYW | | YDASNLETGVPSRFSG | |
| | GQGTLVTVSSSTKGPSVFPLAPCSRSTSES | | SGSGTDFTLTISSLQPE | |
| | TAALGCLVKDYFPEPVTVSWNSGALTSG | | DFATYYCQQANSFPPT | |
| | VHTFPAVLQSSGLYSLSSVVTVPSSSLGTK | | FGGGTKVEIKRTVAAP | |
| | TYTCNVDHKPSNTKVDKRVSKYGPPCPS | | SVFIFPPSDEQLKSGTA | |
| | CPPEFLGGPSVFLFPPKPKDTLMISRTPEV | | SVVCLLNNFYPREAK | |
| | TCVVVDVSQEDPEVQFNWYVDGVEVHN | | VQWKVDNALQSGNS | |
| | AKTKPREEQFNSTYRVVSVLTVLHQDWL | | QESVTEQDSKDSTYSL | |
| | NGKEYKCKVSNKGLPSSIEKTISKAKQPR | | SSTLTLSKADYEKHKV | |
| | EPQVYTLPPSQEEMTKNQVSLTCLVKGFY | | YACEVTHQGLSSPVT | |
| | PSDIAVEWESNGQPENNYKTTPPVLDSDG | | KSFNRGEC | |
| | SFFLYSRLTVDKSRWQEGNVFSCSVMHE | | | |
| | ALHNHYTQKSLSLSLGK | | | |
| 135 | EVQLLESGGGLVQPGGSLRLSCAASGFTF | 1849 | DIVMTQSPLSLPVTPG | 1937 |
| | DEYAMHWVRQAPGKGLEWVSAIGAGGS | | EPASISCRSSQSLLHSN | |
| | TYYADSVKGRFTISRDNSKNTLYLQMNS | | GYNYLDWYLQKPGQS | |
| | LRAEDTAVYYCASSLGPELRGVDYYYYG | | PQLLIYAASSLQSGVP | |
| | MDVWGQGTTVTVSSSTKGPSVFPLAPCS | | DRFSGSGSGTDFTLKIS | |
| | RSTSESTAALGCLVKDYFPEPVTVSWNSG | | RVEAEDVGVYYCMQ | |
| | ALTSGVHTFPAVLQSSGLYSLSSVVTVPSS | | GIQWPWTFGQGTKVE | |
| | SLGTKTYTCNVDHKPSNTKVDKRVSKYG | | IKRTVAAPSVFIFPPSD | |
| | PPCPSCPPEFLGGPSVFLFPPKPKDTLMISR | | EQLKSGTASVVCLLN | |
| | TPEVTCVVVDVSQEDPEVQFNWYVDGVE | | NFYPREAKVQWKVD | |
| | VHNAKTKPREEQFNSTYRVVSVLTVLHQ | | NALQSGNSQESVTEQ | |
| | DWLNGKEYKCKVSNKGLPSSIEKTISKAK | | DSKDSTYSLSSTLTLS | |
| | QPREPQVYTLPPSQEEMTKNQVSLTCLVK | | KADYEKHKVYACEVT | |
| | GFYPSDIAVEWESNGQPENNYKTTPPVLD | | HQGLSSPVTKSFNRGE | |
| | SDGSFFLYSRLTVDKSRWQEGNVFSCSV | | C | |
| | MHEALHNHYTQKSLSLSLGK | | | |
| 136 | EVQLLESGGGLVQPGGSLRLSCAASGFNF | 1850 | DIQMTQSPSSLSASVG | 1938 |
| | DDYAMHWVRQAPGKGLEWVSVIYSGGS | | DRVTITCRASQSISTYV | |
| | TYYADSVKGRFTISRDNSKNTLYLQMNS | | NWYQQKPGKAPKLLI | |
| | LRAEDTAVYYCTRHDFDYWGQGTLVTV | | YAASSLQSGVPSRFSG | |
| | SSSTKGPSVFPLAPCSRSTSESTAALGCLV | | SGSGTDFTLTISSLQPE | |
| | KDYFPEPVTVSWNSGALTSGVHTFPAVL | | DFATYYCQQDYSYPY | |
| | QSSGLYSLSSVVTVPSSSLGTKTYTCNVD | | TFGQGTKVEIKRTVAA | |
| | HKPSNTKVDKRVSKYGPPCPSCPPEFLGG | | PSVFIFPPSDEQLKSGT | |
| | PSVFLFPPKPKDTLMISRTPEVTCVVVDVS | | ASVVCLLNNFYPREA | |
| | QEDPEVQFNWYVDGVEVHNAKTKPREE | | KVQWKVDNALQSGN | |
| | QFNSTYRVVSVLTVLHQDWLNGKEYKC | | SQESVTEQDSKDSTYS | |
| | KVSNKGLPSSIEKTISKAKQPREPQVYTLP | | LSSTLTLSKADYEKHK | |
| | PSQEEMTKNQVSLTCLVKGFYPSDIAVE | | VYACEVTHQGLSSPV | |
| | WESNGQPENNYKTTPPVLDSDGSFFLYSR | | TKSFNRGEC | |
| | LTVDKSRWQEGNVFSCSVMHEALHNHY | | | |
| | TQKSLSLSLGK | | | |
| 137 | EVQLVESGGGLVKPGGSLRLSCAASGFTF | 1851 | DIQMTQSPSSLSASVG | 1939 |
| | SDYALHWVRQAPGKGLEWVSLISGDGGS | | DRVTITCRASQSISTW | |
| | TYYADSVKGRFTISRDDSKNTLYLQMNS | | LAWYQQKPGKAPKLL | |
| | LKTEDTAVYYCARDLGGERSYWGQGTL | | IYAASTLQSGVPSRFS | |
| | VTVSSSTKGPSVFPLAPCSRSTSESTAALG | | GSGSGTDFTLTISSLQP | |
| | CLVKDYFPEPVTVSWNSGALTSGVHTFP | | EDFATYYCLQDYSYPP | |
| | AVLQSSGLYSLSSVVTVPSSSLGTKTYTC | | TFGQGTKVEIKRTVAA | |
| | NVDHKPSNTKVDKRVSKYGPPCPSCPPEF | | PSVFIFPPSDEQLKSGT | |
| | LGGPSVFLFPPKPKDTLMISRTPEVTCVVV | | ASVVCLLNNFYPREA | |
| | DVSQEDPEVQFNWYVDGVEVHNAKTKP | | KVQWKVDNALQSGN | |
| | REEQFNSTYRVVSVLTVLHQDWLNGKEY | | SQESVTEQDSKDSTYS | |
| | KCKVSNKGLPSSIEKTISKAKQPREPQVYT | | LSSTLTLSKADYEKHK | |
| | LPPSQEEMTKNQVSLTCLVKGFYPSDIAV | | VYACEVTHQGLSSPV | |
| | EWESNGQPENNYKTTPPVLDSDGSFFLYS | | TKSFNRGEC | |
| | RLTVDKSRWQEGNVFSCSVMHEALHNH | | | |
| | YTQKSLSLSLGK | | | |
| 138 | QVQLVQSGAEVKKPGASVKVSCKASGYT | 1852 | DIQMTQSPSSLSASVG | 1940 |
| | FTDYYMHWVRQAPGQGLEWMGIINPSD | | DRVTITCRASQSISSW | |
| | GSTTYAQSFQGRVTMTRDTSTSTVYMEL | | LAWYQQKPGKAPKLL | |
| | SSLRSEDTAVYYCARDELPDSSGWYGYF | | IYAASSLQSGVPSRFS | |
| | QHWGQGTLVTVSSSTKGPSVFPLAPCSRS | | GSGSGTDFTLTISSLQP | |
| | TSESTAALGCLVKDYFPEPVTVSWNSGAL | | EDFATYYCQQSYDIPL | |
| | TSGVHTFPAVLQSSGLYSLSSVVTVPSSSL | | TFGGGTKVEIKRTVAA | |
| | GTKTYTCNVDHKPSNTKVDKRVSKYGPP | | PSVFIFPPSDEQLKSGT | |
| | CPSCPPEFLGGPSVFLFPPKPKDTLMISRTP | | ASVVCLLNNFYPREA | |
| | EVTCVVVDVSQEDPEVQFNWYVDGVEV | | KVQWKVDNALQSGN | |
| | HNAKTKPREEQFNSTYRVVSVLTVLHQD | | SQESVTEQDSKDSTYS | |
| | WLNGKEYKCKVSNKGLPSSIEKTISKAKQ | | LSSTLTLSKADYEKHK | |
| | PREPQVYTLPPSQEEMTKNQVSLTCLVKG | | VYACEVTHQGLSSPV | |
| | FYPSDIAVEWESNGQPENNYKTTPPVLDS | | TKSFNRGEC | |
| | DGSFFLYSRLTVDKSRWQEGNVFSCSVM | | | |
| | HEALHNHYTQKSLSLSLGK | | | |
| 139 | QVQLVQSGAEVKKPGSSVKVSCKASGGT | 1853 | DIQMTQSPSSLSASVG | 1941 |
| | FSSYAISWVRQAPGQGLEWMGEIIPFFGT | | DRVTITCQASQDISNL | |
| | ANYAQKFQGRVTITADESTSTAYMELSSL | | LNWYQQKPGKAPKLL | |
| | RSEDTAVYYCARAEYGGDLDYWGQGTL | | IYAASTLQSGVPSRES | |
| | VTVSSSTKGPSVFPLAPCSRSTSESTAALG | | GSGSGTDFTLTISSLQP | |
| | CLVKDYFPEPVTVSWNSGALTSGVHTFP | | EDFATYYCQQSYNTP | |
| | AVLQSSGLYSLSSVVTVPSSSLGTKTYTC | | WTFGPGTKVDIKRTV | |
| | NVDHKPSNTKVDKRVSKYGPPCPSCPPEF | | AAPSVFIFPPSDEQLKS | |
| | LGGPSVFLFPPKPKDTLMISRTPEVTCVVV | | GTASVVCLLNNFYPRE | |
| | DVSQEDPEVQFNWYVDGVEVHNAKTKP | | AKVQWKVDNALQSG | |
| | REEQFNSTYRVVSVLTVLHQDWLNGKEY | | NSQESVTEQDSKDSTY | |
| | KCKVSNKGLPSSIEKTISKAKQPREPQVYT | | SLSSTLTLSKADYEKH | |
| | LPPSQEEMTKNQVSLTCLVKGFYPSDIAV | | KVYACEVTHQGLSSP | |
| | EWESNGQPENNYKTTPPVLDSDGSFFLYS | | VTKSFNRGEC | |
| | RLTVDKSRWQEGNVFSCSVMHEALHNH | | | |
| | YTQKSLSLSLGK | | | |
| 140 | QVQLVQSGAEVKKPGASVKVSCKASGDT | 1854 | DIQMTQSPSSLSASVG | 1942 |
| | FTRHYVHWVRQAPGQGLEWMGIINPRGG | | DRVTITCQASQDIHNY | |
| | THYAQKFQGRVTMTRDTSTSTVYMELSS | | LNWYQQKPGKAPKLL | |
| | LRSEDTAVYYCARRDCSGGSCYSDLDYW | | IYQASSLESGVPSRFSG | |
| | GQGTLVTVSSSTKGPSVFPLAPCSRSTSES | | SGSGTDFTLTISSLQPE | |
| | TAALGCLVKDYFPEPVTVSWNSGALTSG | | DFATYYCQQANSFPLT | |
| | VHTFPAVLQSSGLYSLSSVVTVPSSSLGTK | | FGGGTKLEIKRTVAAP | |
| | TYTCNVDHKPSNTKVDKRVSKYGPPCPS | | SVFIFPPSDEQLKSGTA | |
| | CPPEFLGGPSVFLFPPKPKDTLMISRTPEV | | SVVCLLNNFYPREAK | |
| | TCVVVDVSQEDPEVQFNWYVDGVEVHN | | VQWKVDNALQSGNS | |
| | AKTKPREEQFNSTYRVVSVLTVLHQDWL | | QESVTEQDSKDSTYSL | |
| | NGKEYKCKVSNKGLPSSIEKTISKAKQPR | | SSTLTLSKADYEKHKV | |
| | EPQVYTLPPSQEEMTKNQVSLTCLVKGFY | | YACEVTHQGLSSPVT | |
| | PSDIAVEWESNGQPENNYKTTPPVLDSDG | | KSFNRGEC | |
| | SFFLYSRLTVDKSRWQEGNVFSCSVMHE | | | |
| | ALHNHYTQKSLSLSLGK | | | |
| 141 | QVQLVQSGAEVKKPGASVKVSCKASGGT | 1855 | DIQMTQSPSSLSASVG | 1943 |
| | FSSYAISWVRQAPGQGLEWMGWINPDSG | | DRVTITCRASQNIGSW | |
| | DASYARKFQGRVTMTRDTSTSTVYMELS | | LAWYQQKPGKAPKLL | |
| | SLRSEDTAVYYCATFGEEAFDIWGQGTM | | IYGASILQSGVPSRFSG | |
| | VTVSSSTKGPSVFPLAPCSRSTSESTAALG | | SGSGTDFTLTISSLQPE | |
| | CLVKDYFPEPVTVSWNSGALTSGVHTFP | | DFATYYCQQANSFPLT | |
| | AVLQSSGLYSLSSVVTVPSSSLGTKTYTC | | FGGGTKLEIKRTVAAP | |
| | NVDHKPSNTKVDKRVSKYGPPCPSCPPEF | | SVFIFPPSDEQLKSGTA | |
| | LGGPSVFLFPPKPKDTLMISRTPEVTCVVV | | SVVCLLNNFYPREAK | |
| | DVSQEDPEVQFNWYVDGVEVHNAKTKP | | VQWKVDNALQSGNS | |
| | REEQFNSTYRVVSVLTVLHQDWLNGKEY | | QESVTEQDSKDSTYSL | |
| | KCKVSNKGLPSSIEKTISKAKQPREPQVYT | | SSTLTLSKADYEKHKV | |
| | LPPSQEEMTKNQVSLTCLVKGFYPSDIAV | | YACEVTHQGLSSPVT | |
| | EWESNGQPENNYKTTPPVLDSDGSFFLYS | | KSFNRGEC | |
| | RLTVDKSRWQEGNVFSCSVMHEALHNH | | | |
| | YTQKSLSLSLGK | | | |
| 142 | QVQLVQSGAEVKKPGASVKVSCKASGGT | 1856 | DIQMTQSPSSLSASVG | 1944 |
| | FSSYAISWVRQAPGQGLEWMGWIDPKNG | | DRVTITCRASQGIGNW | |
| | DTNYAQKFQGRVTMTRDTSTSTVYMELS | | LAWYQQKPGKAPKLL | |
| | SLRSEDTAVYYCATEGSHHPYYYYGMDV | | IYEASTLQSGVPSRFSG | |
| | WGQGTTVTVSSSTKGPSVFPLAPCSRSTS | | SGSGTDFTLTISSLQPE | |
| | ESTAALGCLVKDYFPEPVTVSWNSGALTS | | DFATYYCHQYNAYP | |
| | GVHTFPAVLQSSGLYSLSSVVTVPSSSLGT | | WTFGQGTKVEIKRTV | |
| | KTYTCNVDHKPSNTKVDKRVSKYGPPCP | | AAPSVFIFPPSDEQLKS | |
| | SCPPEFLGGPSVFLFPPKPKDTLMISRTPE | | GTASVVCLLNNFYPRE | |
| | VTCVVVDVSQEDPEVQFNWYVDGVEVH | | AKVQWKVDNALQSG | |
| | NAKTKPREEQFNSTYRVVSVLTVLHQDW | | NSQESVTEQDSKDSTY | |
| | LNGKEYKCKVSNKGLPSSIEKTISKAKQP | | SLSSTLTLSKADYEKH | |
| | REPQVYTLPPSQEEMTKNQVSLTCLVKGF | | KVYACEVTHQGLSSP | |
| | YPSDIAVEWESNGQPENNYKTTPPVLDSD | | VTKSFNRGEC | |
| | GSFFLYSRLTVDKSRWQEGNVFSCSVMH | | | |
| | EALHNHYTQKSLSLSLGK | | | |
| 143 | QVQLVQSGAEVKKPGASVKVSCKASGYT | 1857 | DIQMTQSPSSLSASVG | 1945 |
| | FTGYHMHWVRQAPGQGLEWMGWINPN | | DRVTITCQASQDISNY | |
| | TGGTNYAQKFQGRVTMTRDTSTSTVYME | | LNWYQQKPGKAPKLL | |
| | LSSLRSEDTAVYYCARPNTAMVPPYYYY | | IYAASSLQSGVPSRFS | |
| | YGMDVWGQGTLVTVSSSTKGPSVFPLAP | | GSGSGTDFTLTISSLQP | |
| | CSRSTSESTAALGCLVKDYFPEPVTVSWN | | EDFATYYCQQYNSYP | |
| | SGALTSGVHTFPAVLQSSGLYSLSSVVTV | | LTFGQGTKLEIKRTVA | |
| | PSSSLGTKTYTCNVDHKPSNTKVDKRVS | | APSVFIFPPSDEQLKSG | |
| | KYGPPCPSCPPEFLGGPSVFLFPPKPKDTL | | TASVVCLLNNFYPREA | |
| | MISRTPEVTCVVVDVSQEDPEVQFNWYV | | KVQWKVDNALQSGN | |
| | DGVEVHNAKTKPREEQFNSTYRVVSVLT | | SQESVTEQDSKDSTYS | |
| | VLHQDWLNGKEYKCKVSNKGLPSSIEKTI | | LSSTLTLSKADYEKHK | |
| | SKAKQPREPQVYTLPPSQEEMTKNQVSLT | | VYACEVTHQGLSSPV | |
| | CLVKGFYPSDIAVEWESNGQPENNYKTTP | | TKSFNRGEC | |
| | PVLDSDGSFFLYSRLTVDKSRWQEGNVFS | | | |
| | CSVMHEALHNHYTQKSLSLSLGK | | | |
| 144 | QVQLVQSGAEVKKPGASVKVSCKASGYT | 1858 | DIQMTQSPSSLSASVG | 1946 |
| | FTSYDINWVRQAPGQGLEWMGWMNPNS | | DRVTITCRASHSISSW | |
| | GNTGYAQKFQGRVTMTRDTSTSTVYMEL | | LAWYQQKPGKAPKLL | |
| | SSLRSEDTAVYYCARVSATGTYGLDYWG | | IYDASNLETGVPSRFS | |
| | QGTLVTVSSSTKGPSVFPLAPCSRSTSEST | | GSGSGTDFTLTISSLQP | |
| | AALGCLVKDYFPEPVTVSWNSGALTSGV | | EDFATYYCQQADSFPL | |
| | HTFPAVLQSSGLYSLSSVVTVPSSSLGTKT | | TFGGGTKVEIKRTVAA | |
| | YTCNVDHKPSNTKVDKRVSKYGPPCPSC | | PSVFIFPPSDEQLKSGT | |
| | PPEFLGGPSVFLFPPKPKDTLMISRTPEVT | | ASVVCLLNNFYPREA | |
| | CVVVDVSQEDPEVQFNWYVDGVEVHNA | | KVQWKVDNALQSGN | |
| | KTKPREEQFNSTYRVVSVLTVLHQDWLN | | SQESVTEQDSKDSTYS | |
| | GKEYKCKVSNKGLPSSIEKTISKAKQPREP | | LSSTLTLSKADYEKHK | |
| | QVYTLPPSQEEMTKNQVSLTCLVKGFYPS | | VYACEVTHQGLSSPV | |
| | DIAVEWESNGQPENNYKTTPPVLDSDGSF | | TKSFNRGEC | |
| | FLYSRLTVDKSRWQEGNVFSCSVMHEAL | | | |
| | HNHYTQKSLSLSLGK | | | |
| 145 | QVQLVQSGAEVKKPGASVKVSCKASGYT | 1859 | DIQMTQSPSSLSASVG | 1947 |
| | FNNYGITWVRQAPGQGLEWMGIINPITGV | | DRVTITCQASQDINDY | |
| | TTYAQNFQGRVTMTRDTSTSTVYMELSS | | LNWYQQKPGKAPKLL | |
| | LRSEDTAVYYCASGEQQLVLFDYWGQGT | | IYGASNLQSGVPSRFS | |
| | LVTVSSSTKGPSVFPLAPCSRSTSESTAAL | | GSGSGTDFTLTISSLQP | |
| | GCLVKDYFPEPVTVSWNSGALTSGVHTF | | EDFATYYCLQHNSYP | |
| | PAVLQSSGLYSLSSVVTVPSSSLGTKTYTC | | LTFGQGTKLEIKRTVA | |
| | NVDHKPSNTKVDKRVSKYGPPCPSCPPEF | | APSVFIFPPSDEQLKSG | |
| | LGGPSVFLFPPKPKDTLMISRTPEVTCVVV | | TASVVCLLNNFYPREA | |
| | DVSQEDPEVQFNWYVDGVEVHNAKTKP | | KVQWKVDNALQSGN | |
| | REEQFNSTYRVVSVLTVLHQDWLNGKEY | | SQESVTEQDSKDSTYS | |
| | KCKVSNKGLPSSIEKTISKAKQPREPQVYT | | LSSTLTLSKADYEKHK | |
| | LPPSQEEMTKNQVSLTCLVKGFYPSDIAV | | VYACEVTHQGLSSPV | |
| | EWESNGQPENNYKTTPPVLDSDGSFFLYS | | TKSFNRGEC | |
| | RLTVDKSRWQEGNVFSCSVMHEALHNH | | | |
| | YTQKSLSLSLGK | | | |
| 146 | QVQLVQSGAEVKKPGASVKVSCKASGYT | 1860 | DIQMTQSPSSLSASVG | 1948 |
| | FTDYYLHWVRQAPGQGLEWMGWMNPN | | DRVTITCRASQGISNY | |
| | SGNTGYAQKFQGRVTMTRDTSTSTVYME | | LAWYQQKPGKAPKLL | |
| | LSSLRSEDTAVYYCAADVITAYGMDVWG | | IYDASNLETGVPSRFS | |
| | QGTMVTVSSSTKGPSVFPLAPCSRSTSEST | | GSGSGTDFTLTISSLQP | |
| | AALGCLVKDYFPEPVTVSWNSGALTSGV | | EDFATYYCQQSYNVP | |
| | HTFPAVLQSSGLYSLSSVVTVPSSSLGTKT | | PTFGQGTKVEIKRTVA | |
| | YTCNVDHKPSNTKVDKRVSKYGPPCPSC | | APSVFIFPPSDEQLKSG | |
| | PPEFLGGPSVFLFPPKPKDTLMISRTPEVT | | TASVVCLLNNFYPREA | |
| | CVVVDVSQEDPEVQFNWYVDGVEVHNA | | KVQWKVDNALQSGN | |
| | KTKPREEQFNSTYRVVSVLTVLHQDWLN | | SQESVTEQDSKDSTYS | |
| | GKEYKCKVSNKGLPSSIEKTISKAKQPREP | | LSSTLTLSKADYEKHK | |
| | QVYTLPPSQEEMTKNQVSLTCLVKGFYPS | | VYACEVTHQGLSSPV | |
| | DIAVEWESNGQPENNYKTTPPVLDSDGSF | | TKSFNRGEC | |
| | FLYSRLTVDKSRWQEGNVFSCSVMHEAL | | | |
| | HNHYTQKSLSLSLGK | | | |
| 147 | EVQLLESGGGLVQPGGSLRLSCAASGFTF | 1861 | DIQMTQSPSSLSASVG | 1949 |
| | SNAWMSWVRQAPGKGLEWVADISYDGT | | DRVTITCRASQSISSYL | |
| | NDYYADSVKGRFTISRDNSKNTLYLQMN | | NWYQQKPGKAPKLLI | |
| | SLRAEDTAVYYCTTEELRFGGFDYWGQG | | YDASNLETGVPSRFSG | |
| | TLVTVSSSTKGPSVFPLAPCSRSTSESTAA | | SGSGTDFTLTISSLQPE | |
| | LGCLVKDYFPEPVTVSWNSGALTSGVHT | | DFATYYCQQANSFPLT | |
| | FPAVLQSSGLYSLSSVVTVPSSSLGTKTYT | | FGQGTKVEIKRTVAAP | |
| | CNVDHKPSNTKVDKRVSKYGPPCPSCPPE | | SVFIFPPSDEQLKSGTA | |
| | FLGGPSVFLFPPKPKDTLMISRTPEVTCVV | | SVVCLLNNFYPREAK | |
| | VDVSQEDPEVQFNWYVDGVEVHNAKTK | | VQWKVDNALQSGNS | |
| | PREEQFNSTYRVVSVLTVLHQDWLNGKE | | QESVTEQDSKDSTYSL | |
| | YKCKVSNKGLPSSIEKTISKAKQPREPQV | | SSTLTLSKADYEKHKV | |
| | YTLPPSQEEMTKNQVSLTCLVKGFYPSDI | | YACEVTHQGLSSPVT | |
| | AVEWESNGQPENNYKTTPPVLDSDGSFFL | | KSFNRGEC | |
| | YSRLTVDKSRWQEGNVFSCSVMHEALHN | | | |
| | HYTQKSLSLSLGK | | | |
| 148 | QVQLVQSGAEVKKPGSSVKVSCKASGGT | 1862 | EIVMTQSPATLSVSPG | 1950 |
| | FSSYAISWVRQAPGQGLEWMGGIIPMFGT | | ERATLSCRASQSIGTY | |
| | ANYAQKFQGRVTITADESTSTAYMELSSL | | LAWYQQKPGQAPRLL | |
| | RSEDTAVYYCARDLGYSNAGGTLHYWG | | IYDASSRATGIPARFSG | |
| | QGTLVTVSSSTKGPSVFPLAPCSRSTSEST | | SGSGTEFTLTISSLQSE | |
| | AALGCLVKDYFPEPVTVSWNSGALTSGV | | DFAVYYCQQYKSYPL | |
| | HTFPAVLQSSGLYSLSSVVTVPSSSLGTKT | | TFGGGTKVEIKRTVAA | |
| | YTCNVDHKPSNTKVDKRVSKYGPPCPSC | | PSVFIFPPSDEQLKSGT | |
| | PPEFLGGPSVFLFPPKPKDTLMISRTPEVT | | ASVVCLLNNFYPREA | |
| | CVVVDVSQEDPEVQFNWYVDGVEVHNA | | KVQWKVDNALQSGN | |
| | KTKPREEQFNSTYRVVSVLTVLHQDWLN | | SQESVTEQDSKDSTYS | |
| | GKEYKCKVSNKGLPSSIEKTISKAKQPREP | | LSSTLTLSKADYEKHK | |
| | QVYTLPPSQEEMTKNQVSLTCLVKGFYPS | | VYACEVTHQGLSSPV | |
| | DIAVEWESNGQPENNYKTTPPVLDSDGSF | | TKSFNRGEC | |
| | FLYSRLTVDKSRWQEGNVFSCSVMHEAL | | | |
| | HNHYTQKSLSLSLGK | | | |
| 149 | QVQLVQSGAEVKKPGASVKVSCKASGYT | 1863 | DIQMTQSPSSLSASVG | 1951 |
| | FTNYYMHWVRQAPGQGLEWMGIINPSG | | DRVTITCQASQDISNY | |
| | GSTSYAQKFQGRVTMTRDTSTSTVYMEL | | LNWYQQKPGKAPKLL | |
| | SSLRSEDTAVYYCARAEWDILTGYYIDY | | IYGASSLQSGVPSRFS | |
| | WGQGTLVTVSSSTKGPSVFPLAPCSRSTS | | GSGSGTDFTLTISSLQP | |
| | ESTAALGCLVKDYFPEPVTVSWNSGALTS | | EDFATYYCQQHNSYP | |
| | GVHTFPAVLQSSGLYSLSSVVTVPSSSLGT | | WTFGQGTKVEIKRTV | |
| | KTYTCNVDHKPSNTKVDKRVSKYGPPCP | | AAPSVFIFPPSDEQLKS | |
| | SCPPEFLGGPSVFLFPPKPKDTLMISRTPE | | GTASVVCLLNNFYPRE | |
| | VTCVVVDVSQEDPEVQFNWYVDGVEVH | | AKVQWKVDNALQSG | |
| | NAKTKPREEQFNSTYRVVSVLTVLHQDW | | NSQESVTEQDSKDSTY | |
| | LNGKEYKCKVSNKGLPSSIEKTISKAKQP | | SLSSTLTLSKADYEKH | |
| | REPQVYTLPPSQEEMTKNQVSLTCLVKGF | | KVYACEVTHQGLSSP | |
| | YPSDIAVEWESNGQPENNYKTTPPVLDSD | | VTKSFNRGEC | |
| | GSFFLYSRLTVDKSRWQEGNVFSCSVMH | | | |
| | EALHNHYTQKSLSLSLGK | | | |
| 150 | QVQLVQSGAEVKKPGASVKVSCKASGYT | 1864 | DIQMTQSPSSLSASVG | 1952 |
| | FTDHFVHWVRQAPGQGLEWMGWISAYN | | DRVTITCRASQGIHNY | |
| | GNTNYAQKFQGRVTMTRDTSTSTVYMEL | | LAWYQQKPGKAPKLL | |
| | SSLRSEDTAVYYCARAEYSYGFDYWGQG | | IYDASNLETGVPSRFS | |
| | TLVTVSSSTKGPSVFPLAPCSRSTSESTAA | | GSGSGTDFTLTISSLQP | |
| | LGCLVKDYFPEPVTVSWNSGALTSGVHT | | EDFATYYCQQTSSFPY | |
| | FPAVLQSSGLYSLSSVVTVPSSSLGTKTYT | | TFGQGTKLEIKRTVAA | |
| | CNVDHKPSNTKVDKRVSKYGPPCPSCPPE | | PSVFIFPPSDEQLKSGT | |
| | FLGGPSVFLFPPKPKDTLMISRTPEVTCVV | | ASVVCLLNNFYPREA | |
| | VDVSQEDPEVQFNWYVDGVEVHNAKTK | | KVQWKVDNALQSGN | |
| | PREEQFNSTYRVVSVLTVLHQDWLNGKE | | SQESVTEQDSKDSTYS | |
| | YKCKVSNKGLPSSIEKTISKAKQPREPQV | | LSSTLTLSKADYEKHK | |
| | YTLPPSQEEMTKNQVSLTCLVKGFYPSDI | | VYACEVTHQGLSSPV | |
| | AVEWESNGQPENNYKTTPPVLDSDGSFFL | | TKSFNRGEC | |
| | YSRLTVDKSRWQEGNVFSCSVMHEALHN | | | |
| | HYTQKSLSLSLGK | | | |
| 151 | QVQLVQSGAEVKKPGASVKVSCKASGYT | 1865 | DIQMTQSPSSLSASVG | 1953 |
| | FTGYYVHWVRQAPGQGLEWMGVINPSG | | DRVTITCQASQDISNY | |
| | GGSPSYAQKFQGRVTMTRDTSTSTVYME | | LNWYQQKPGKAPKLL | |
| | LSSLRSEDTAVYYCARDRSDVDYGMDV | | IYDASNLQSGVPSRFS | |
| | WGQGTTVTVSSSTKGPSVFPLAPCSRSTS | | GSGSGTDFTLTISSLQP | |
| | ESTAALGCLVKDYFPEPVTVSWNSGALTS | | EDFATYYCLQHNSYP | |
| | GVHTFPAVLQSSGLYSLSSVVTVPSSSLGT | | LTFGGGTKVEIKRTVA | |
| | KTYTCNVDHKPSNTKVDKRVSKYGPPCP | | APSVFIFPPSDEQLKSG | |
| | SCPPEFLGGPSVFLFPPKPKDTLMISRTPE | | TASVVCLLNNFYPREA | |
| | VTCVVVDVSQEDPEVQFNWYVDGVEVH | | KVQWKVDNALQSGN | |
| | NAKTKPREEQFNSTYRVVSVLTVLHQDW | | SQESVTEQDSKDSTYS | |
| | LNGKEYKCKVSNKGLPSSIEKTISKAKQP | | LSSTLTLSKADYEKHK | |
| | REPQVYTLPPSQEEMTKNQVSLTCLVKGF | | VYACEVTHQGLSSPV | |
| | YPSDIAVEWESNGQPENNYKTTPPVLDSD | | TKSFNRGEC | |
| | GSFFLYSRLTVDKSRWQEGNVFSCSVMH | | | |
| | EALHNHYTQKSLSLSLGK | | | |
| 152 | QVQLVQSGAEVKKPGASVKVSCKASGYT | 1866 | DIVMTQSPLSLPVTPG | 1954 |
| | FTDYYMHWVRQAPGQGLEWMGLIDPSG | | EPASISCRSSQSLLHSN | |
| | GSTNSLQKFQGRVTMTRDTSTSTVYMEL | | GYNYLDWYLQKPGQS | |
| | SSLRSEDTAVYYCARDVGFGELSFDIWGQ | | PQLLIYAASTLQSGVP | |
| | GTTVTVSSSTKGPSVFPLAPCSRSTSESTA | | DRFSGSGSGTDFTLKIS | |
| | ALGCLVKDYFPEPVTVSWNSGALTSGVH | | RVEAEDVGVYYCMQ | |
| | TFPAVLQSSGLYSLSSVVTVPSSSLGTKTY | | GTHWPPTFGPGTKVDI | |
| | TCNVDHKPSNTKVDKRVSKYGPPCPSCPP | | KRTVAAPSVFIFPPSDE | |
| | EFLGGPSVFLFPPKPKDTLMISRTPEVTCV | | QLKSGTASVVCLLNN | |
| | VVDVSQEDPEVQFNWYVDGVEVHNAKT | | FYPREAKVQWKVDN | |
| | KPREEQFNSTYRVVSVLTVLHQDWLNGK | | ALQSGNSQESVTEQDS | |
| | EYKCKVSNKGLPSSIEKTISKAKQPREPQV | | KDSTYSLSSTLTLSKA | |
| | YTLPPSQEEMTKNQVSLTCLVKGFYPSDI | | DYEKHKVYACEVTHQ | |
| | AVEWESNGQPENNYKTTPPVLDSDGSFFL | | GLSSPVTKSFNRGEC | |
| | YSRLTVDKSRWQEGNVFSCSVMHEALHN | | | |
| | HYTQKSLSLSLGK | | | |
| 153 | QVQLVQSGAEVKKPGASVKVSCKASGYT | 1867 | DIQMTQSPSSLSASVG | 1955 |
| | FTGYYMHWVRQAPGQGLEWMGWINPNS | | DRVTITCRASQSIGTY | |
| | GGTNYAQKFQGRVTMTRDTSTSTVYMEL | | LNWYQQKPGKAPKLL | |
| | SSLRSEDTAVYYCAREIGGYDNYYYYGM | | IYAASSLQSGVPSRFS | |
| | DVWGQGTTVTVSSSTKGPSVFPLAPCSRS | | GSGSGTDFTLTISSLQP | |
| | TSESTAALGCLVKDYFPEPVTVSWNSGAL | | EDFATYYCQQSYTDP | |
| | TSGVHTFPAVLQSSGLYSLSSVVTVPSSSL | | WTFGQGTKVEIKRTV | |
| | GTKTYTCNVDHKPSNTKVDKRVSKYGPP | | AAPSVFIFPPSDEQLKS | |
| | CPSCPPEFLGGPSVFLFPPKPKDTLMISRTP | | GTASVVCLLNNFYPRE | |
| | EVTCVVVDVSQEDPEVQFNWYVDGVEV | | AKVQWKVDNALQSG | |
| | HNAKTKPREEQFNSTYRVVSVLTVLHQD | | NSQESVTEQDSKDSTY | |
| | WLNGKEYKCKVSNKGLPSSIEKTISKAKQ | | SLSSTLTLSKADYEKH | |
| | PREPQVYTLPPSQEEMTKNQVSLTCLVKG | | KVYACEVTHQGLSSP | |
| | FYPSDIAVEWESNGQPENNYKTTPPVLDS | | VTKSFNRGEC | |
| | DGSFFLYSRLTVDKSRWQEGNVFSCSVM | | | |
| | HEALHNHYTQKSLSLSLGK | | | |
| 154 | QVQLVQSGAEVKKPGASVKVSCKASGYT | 1868 | DIQMTQSPSSLSASVG | 1956 |
| | FNTYYMHWVRQAPGQGLEWMGWMHPN | | DRVTITCRASQSIFSYL | |
| | TGNTGYAQKFQGRVTMTRDTSTSTVYME | | NWYQQKPGKAPKLLI | |
| | LSSLRSEDTAVYYCARGTTSDAFDIWGQ | | YSASNLQSGVPSRFSG | |
| | GTMVTVSSSTKGPSVFPLAPCSRSTSESTA | | SGSGTDFTLTISSLQPE | |
| | ALGCLVKDYFPEPVTVSWNSGALTSGVH | | DFATYYCQQSYSTPIT | |
| | TFPAVLQSSGLYSLSSVVTVPSSSLGTKTY | | FGQGTKVEIKRTVAAP | |
| | TCNVDHKPSNTKVDKRVSKYGPPCPSCPP | | SVFIFPPSDEQLKSGTA | |
| | EFLGGPSVFLFPPKPKDTLMISRTPEVTCV | | SVVCLLNNFYPREAK | |
| | VVDVSQEDPEVQFNWYVDGVEVHNAKT | | VQWKVDNALQSGNS | |
| | KPREEQFNSTYRVVSVLTVLHQDWLNGK | | QESVTEQDSKDSTYSL | |
| | EYKCKVSNKGLPSSIEKTISKAKQPREPQV | | SSTLTLSKADYEKHKV | |
| | YTLPPSQEEMTKNQVSLTCLVKGFYPSDI | | YACEVTHQGLSSPVT | |
| | AVEWESNGQPENNYKTTPPVLDSDGSFFL | | KSFNRGEC | |
| | YSRLTVDKSRWQEGNVFSCSVMHEALHN | | | |
| | HYTQKSLSLSLGK | | | |
| 155 | QVQLVQSGAEVKKPGASVKVSCKASGDT | 1869 | DIQMTQSPSSLSASVG | 1957 |
| | FTRHYVHWVRQAPGQGLEWMGRVNPRD | | DRVTITCRASQGISSYL | |
| | GRTNSAQKFQGRVTMTRDTSTSTVYMEL | | AWYQQKPGKAPKLLI | |
| | SSLRSEDTAVYYCAKDMFPTVTGTYYYY | | YDASNLETGVPSRFSG | |
| | GMDVWGQGTTVTVSSSTKGPSVFPLAPC | | SGSGTDFTLTISSLQPE | |
| | SRSTSESTAALGCLVKDYFPEPVTVSWNS | | DFATYYCQQASGFPY | |
| | GALTSGVHTFPAVLQSSGLYSLSSVVTVP | | TFGQGTRLEIKRTVAA | |
| | SSSLGTKTYTCNVDHKPSNTKVDKRVSK | | PSVFIFPPSDEQLKSGT | |
| | YGPPCPSCPPEFLGGPSVFLFPPKPKDTLM | | ASVVCLLNNFYPREA | |
| | ISRTPEVTCVVVDVSQEDPEVQFNWYVD | | KVQWKVDNALQSGN | |
| | GVEVHNAKTKPREEQFNSTYRVVSVLTV | | SQESVTEQDSKDSTYS | |
| | LHQDWLNGKEYKCKVSNKGLPSSIEKTIS | | LSSTLTLSKADYEKHK | |
| | KAKQPREPQVYTLPPSQEEMTKNQVSLT | | VYACEVTHQGLSSPV | |
| | CLVKGFYPSDIAVEWESNGQPENNYKTTP | | TKSFNRGEC | |
| | PVLDSDGSFFLYSRLTVDKSRWQEGNVFS | | | |
| | CSVMHEALHNHYTQKSLSLSLGK | | | |
| 156 | QVQLVQSGAEVKKPGASVKVSCKASGYT | 1870 | DIQMTQSPSSLSASVG | 1958 |
| | FSSYDINWVRQAPGQGLEWVGWINPRNG | | DRVTITCRASQSISNYL | |
| | GTDYAQKFQGRVTMTRDTSTSTVYMELS | | NWYQQKPGKAPKLLI | |
| | SLRSEDTAVYYCARHRWELDSFDYWGQ | | YATSSLQSGVPSRFSG | |
| | GTLVTVSSSTKGPSVFPLAPCSRSTSESTA | | SGSGTDFTLTISSLQPE | |
| | ALGCLVKDYFPEPVTVSWNSGALTSGVH | | DFATYYCQQGYNIPFT | |
| | TFPAVLQSSGLYSLSSVVTVPSSSLGTKTY | | FGQGTKLEIKRTVAAP | |
| | TCNVDHKPSNTKVDKRVSKYGPPCPSCPP | | SVFIFPPSDEQLKSGTA | |
| | EFLGGPSVFLFPPKPKDTLMISRTPEVTCV | | SVVCLLNNFYPREAK | |
| | VVDVSQEDPEVQFNWYVDGVEVHNAKT | | VQWKVDNALQSGNS | |
| | KPREEQFNSTYRVVSVLTVLHQDWLNGK | | QESVTEQDSKDSTYSL | |
| | EYKCKVSNKGLPSSIEKTISKAKQPREPQV | | SSTLTLSKADYEKHKV | |
| | YTLPPSQEEMTKNQVSLTCLVKGFYPSDI | | YACEVTHQGLSSPVT | |
| | AVEWESNGQPENNYKTTPPVLDSDGSFFL | | KSFNRGEC | |
| | YSRLTVDKSRWQEGNVFSCSVMHEALHN | | | |
| | HYTQKSLSLSLGK | | | |
| 157 | QVQLVQSGAEVKKPGASVKVSCKASGYT | 1871 | DIQMTQSPSSLSASVG | 1959 |
| | FTSYYIHWVRQAPGQGLEWMGWMNPND | | DRVTITCRASESISGW | |
| | GKTAYAQRFQGRVTMTRDTSTSTVYMEL | | LAWYQQKPGKAPKLL | |
| | SSLRSEDTAVYYCARDDDYGGYVAYWG | | IYDASNLETGVPSRFS | |
| | QGTLVTVSSSTKGPSVFPLAPCSRSTSEST | | GSGSGTDFTLTISSLQP | |
| | AALGCLVKDYFPEPVTVSWNSGALTSGV | | EDFATYYCQQYDTWP | |
| | HTFPAVLQSSGLYSLSSVVTVPSSSLGTKT | | FTFGPGTKVDIKRTVA | |
| | YTCNVDHKPSNTKVDKRVSKYGPPCPSC | | APSVFIFPPSDEQLKSG | |
| | PPEFLGGPSVFLFPPKPKDTLMISRTPEVT | | TASVVCLLNNFYPREA | |
| | CVVVDVSQEDPEVQFNWYVDGVEVHNA | | KVQWKVDNALQSGN | |
| | KTKPREEQFNSTYRVVSVLTVLHQDWLN | | SQESVTEQDSKDSTYS | |
| | GKEYKCKVSNKGLPSSIEKTISKAKQPREP | | LSSTLTLSKADYEKHK | |
| | QVYTLPPSQEEMTKNQVSLTCLVKGFYPS | | VYACEVTHQGLSSPV | |
| | DIAVEWESNGQPENNYKTTPPVLDSDGSF | | TKSFNRGEC | |
| | FLYSRLTVDKSRWQEGNVFSCSVMHEAL | | | |
| | HNHYTQKSLSLSLGK | | | |
| 158 | EVQLLESGGGLVQPGGSLRLSCAASGMS | 1872 | DIQMTQSPSSLSASVG | 1960 |
| | VTSNHMSWVRQAPGKGLEWVSSIYPDGK | | DRVTITCQASQSISNW | |
| | TYYADSVKGRFTISRDNSKNTLYLQMNS | | LAWYQQKPGKAPKLL | |
| | LRAEDTAVYYCARDEEDWFDPWGQGTL | | IYAASTLQSGVPSRFS | |
| | VTVSSSTKGPSVFPLAPCSRSTSESTAALG | | GSGSGTDFTLTISSLQP | |
| | CLVKDYFPEPVTVSWNSGALTSGVHTFP | | EDFATYYCQQSYSTP | |
| | AVLQSSGLYSLSSVVTVPSSSLGTKTYTC | | WTFGQGTKVEIKRTV | |
| | NVDHKPSNTKVDKRVSKYGPPCPSCPPEF | | AAPSVFIFPPSDEQLKS | |
| | LGGPSVFLFPPKPKDTLMISRTPEVTCVVV | | GTASVVCLLNNFYPRE | |
| | DVSQEDPEVQFNWYVDGVEVHNAKTKP | | AKVQWKVDNALQSG | |
| | REEQFNSTYRVVSVLTVLHQDWLNGKEY | | NSQESVTEQDSKDSTY | |
| | KCKVSNKGLPSSIEKTISKAKQPREPQVYT | | SLSSTLTLSKADYEKH | |
| | LPPSQEEMTKNQVSLTCLVKGFYPSDIAV | | KVYACEVTHQGLSSP | |
| | EWESNGQPENNYKTTPPVLDSDGSFFLYS | | VTKSFNRGEC | |
| | RLTVDKSRWQEGNVFSCSVMHEALHNH | | | |
| | YTQKSLSLSLGK | | | |
| 159 | EVQLLESGGGLVQPGGSLRLSCAASGFTF | 1873 | DIQMTQSPSSLSASVG | 1961 |
| | SNHYMSWVRQAPGKGLEWVAVIWPDGS | | DRVTITCQASQDISNY | |
| | KEYYADSVKGRFTISRDNSKNTLYLQMN | | LNWYQQKPGKAPKLL | |
| | SLRAEDTAVYYCAREDYYGSGMDYWGQ | | IYGASTLQSGVPSRFS | |
| | GTLVTVSSSTKGPSVFPLAPCSRSTSESTA | | GSGSGTDFTLTISSLQP | |
| | ALGCLVKDYFPEPVTVSWNSGALTSGVH | | EDFATYYCQQYDSYP | |
| | TFPAVLQSSGLYSLSSVVTVPSSSLGTKTY | | PTFGGGTKVEIKRTVA | |
| | TCNVDHKPSNTKVDKRVSKYGPPCPSCPP | | APSVFIFPPSDEQLKSG | |
| | EFLGGPSVFLFPPKPKDTLMISRTPEVTCV | | TASVVCLLNNFYPREA | |
| | VVDVSQEDPEVQFNWYVDGVEVHNAKT | | KVQWKVDNALQSGN | |
| | KPREEQFNSTYRVVSVLTVLHQDWLNGK | | SQESVTEQDSKDSTYS | |
| | EYKCKVSNKGLPSSIEKTISKAKQPREPQV | | LSSTLTLSKADYEKHK | |
| | YTLPPSQEEMTKNQVSLTCLVKGFYPSDI | | VYACEVTHQGLSSPV | |
| | AVEWESNGQPENNYKTTPPVLDSDGSFFL | | TKSFNRGEC | |
| | YSRLTVDKSRWQEGNVFSCSVMHEALHN | | | |
| | HYTQKSLSLSLGK | | | |
| 160 | QVQLVQSGAEVKKPGASVKVSCKASGGT | 1874 | DIQMTQSPSSLSASVG | 1962 |
| | FSNYAISWVRQAPGQGLEWMGWISAYN | | DRVTITCQASEDINKY | |
| | GNSDYAQNLQGRVTMTRDTSTSTVYMEL | | LNWYQQKPGKAPKLL | |
| | SSLRSEDTAVYYCAIGDYFDYWGQGTLV | | IYDASNLETGVPSRFS | |
| | TVSSSTKGPSVFPLAPCSRSTSESTAALGC | | GSGSGTDFTLTISSLQP | |
| | LVKDYFPEPVTVSWNSGALTSGVHTFPA | | EDFATYYCQQANSFPL | |
| | VLQSSGLYSLSSVVTVPSSSLGTKTYTCN | | TFGQGTKVEIKRTVAA | |
| | VDHKPSNTKVDKRVSKYGPPCPSCPPEFL | | PSVFIFPPSDEQLKSGT | |
| | GGPSVFLFPPKPKDTLMISRTPEVTCVVV | | ASVVCLLNNFYPREA | |
| | DVSQEDPEVQFNWYVDGVEVHNAKTKP | | KVQWKVDNALQSGN | |
| | REEQFNSTYRVVSVLTVLHQDWLNGKEY | | SQESVTEQDSKDSTYS | |
| | KCKVSNKGLPSSIEKTISKAKQPREPQVYT | | LSSTLTLSKADYEKHK | |
| | LPPSQEEMTKNQVSLTCLVKGFYPSDIAV | | VYACEVTHQGLSSPV | |
| | EWESNGQPENNYKTTPPVLDSDGSFFLYS | | TKSFNRGEC | |
| | RLTVDKSRWQEGNVFSCSVMHEALHNH | | | |
| | YTQKSLSLSLGK | | | |
| 161 | EVQLLESGGGLVQPGGSLRLSCAASGFTV | 1875 | DIQMTQSPSSLSASVG | 1963 |
| | SSNYMSWVRQAPGKGLEWVAVIYSDGK | | DRVTITCRASQSISTYL | |
| | TYYADSVKGRFTISRDNSKNTLYLQMNS | | NWYQQKPGKAPKLLI | |
| | LRAEDTAVYYCAREDSSGSHFDYWGQGT | | YDASNLETGVPSRFSG | |
| | LVTVSSSTKGPSVFPLAPCSRSTSESTAAL | | SGSGTDFTLTISSLQPE | |
| | GCLVKDYFPEPVTVSWNSGALTSGVHTF | | DFATYYCQQAHSFPPT | |
| | PAVLQSSGLYSLSSVVTVPSSSLGTKTYTC | | FGQGTRLEIKRTVAAP | |
| | NVDHKPSNTKVDKRVSKYGPPCPSCPPEF | | SVFIFPPSDEQLKSGTA | |
| | LGGPSVFLFPPKPKDTLMISRTPEVTCVVV | | SVVCLLNNFYPREAK | |
| | DVSQEDPEVQFNWYVDGVEVHNAKTKP | | VQWKVDNALQSGNS | |
| | REEQFNSTYRVVSVLTVLHQDWLNGKEY | | QESVTEQDSKDSTYSL | |
| | KCKVSNKGLPSSIEKTISKAKQPREPQVYT | | SSTLTLSKADYEKHKV | |
| | LPPSQEEMTKNQVSLTCLVKGFYPSDIAV | | YACEVTHQGLSSPVT | |
| | EWESNGQPENNYKTTPPVLDSDGSFFLYS | | KSFNRGEC | |
| | RLTVDKSRWQEGNVFSCSVMHEALHNH | | | |
| | YTQKSLSLSLGK | | | |
| 162 | QVQLVQSGAEVKKPGSSVKVSCKASGYT | 1876 | DIQMTQSPSSLSASVG | 1964 |
| | FTKYEINWVRQAPGQGLEWMGGIIPIFGT | | DRVTITCRASQGISNN | |
| | ANYAQKFQGRVTITADESTSTAYMELSSL | | LNWYQQKPGKAPKLL | |
| | RSEDTAVYYCARGSGWYTPLFDYWGQG | | IYDASYLETGVPSRFS | |
| | TLVTVSSSTKGPSVFPLAPCSRSTSESTAA | | GSGSGTDFTLTISSLQP | |
| | LGCLVKDYFPEPVTVSWNSGALTSGVHT | | EDFATYYCQQSYSAPL | |
| | FPAVLQSSGLYSLSSVVTVPSSSLGTKTYT | | TFGQGTKVEIKRTVAA | |
| | CNVDHKPSNTKVDKRVSKYGPPCPSCPPE | | PSVFIFPPSDEQLKSGT | |
| | FLGGPSVFLFPPKPKDTLMISRTPEVTCVV | | ASVVCLLNNFYPREA | |
| | VDVSQEDPEVQFNWYVDGVEVHNAKTK | | KVQWKVDNALQSGN | |
| | PREEQFNSTYRVVSVLTVLHQDWLNGKE | | SQESVTEQDSKDSTYS | |
| | YKCKVSNKGLPSSIEKTISKAKQPREPQV | | LSSTLTLSKADYEKHK | |
| | YTLPPSQEEMTKNQVSLTCLVKGFYPSDI | | VYACEVTHQGLSSPV | |
| | AVEWESNGQPENNYKTTPPVLDSDGSFFL | | TKSFNRGEC | |
| | YSRLTVDKSRWQEGNVFSCSVMHEALHN | | | |
| | HYTQKSLSLSLGK | | | |
| 163 | QVQLVQSGAEVKKPGASVKVSCKASGYT | 1877 | EIVMTQSPATLSVSPG | 1965 |
| | FTDYYIHWVRQAPGQGLEWMGLIDPSGG | | ERATLSCRASQSVSSY | |
| | STSIAQKFQGRVTMTRDTSTSTVYMELSS | | LAWYQQKPGQAPRLL | |
| | LRSEDTAVYYCARDYDILTGSGFDPWGQ | | IYDASARATGIPARFS | |
| | GTLVTVSSSTKGPSVFPLAPCSRSTSESTA | | GSGSGTEFTLTISSLQS | |
| | ALGCLVKDYFPEPVTVSWNSGALTSGVH | | EDFAVYYCQQYRSSV | |
| | TFPAVLQSSGLYSLSSVVTVPSSSLGTKTY | | TFGQGTRLEIKRTVAA | |
| | TCNVDHKPSNTKVDKRVSKYGPPCPSCPP | | PSVFIFPPSDEQLKSGT | |
| | EFLGGPSVFLFPPKPKDTLMISRTPEVTCV | | ASVVCLLNNFYPREA | |
| | VVDVSQEDPEVQFNWYVDGVEVHNAKT | | KVQWKVDNALQSGN | |
| | KPREEQFNSTYRVVSVLTVLHQDWLNGK | | SQESVTEQDSKDSTYS | |
| | EYKCKVSNKGLPSSIEKTISKAKQPREPQV | | LSSTLTLSKADYEKHK | |
| | YTLPPSQEEMTKNQVSLTCLVKGFYPSDI | | VYACEVTHQGLSSPV | |
| | AVEWESNGQPENNYKTTPPVLDSDGSFFL | | TKSFNRGEC | |
| | YSRLTVDKSRWQEGNVFSCSVMHEALHN | | | |
| | HYTQKSLSLSLGK | | | |
| 164 | QVQLVQSGAEVKKPGASVKVSCKASGYT | 1878 | DIQMTQSPSSLSASVG | 1966 |
| | FTTYYMHWVRQAPGQGLEWMGIINVSA | | DRVTITCQASQDINNY | |
| | GTTSYAQKFQGRVTMTRDTSTSTVYMEL | | LNWYQQKPGKAPKLL | |
| | SSLRSEDTAVYYCAKEPYPHQSGWFFDY | | IYDASNLETGVPSRFS | |
| | WGQGTLVTVSSSTKGPSVFPLAPCSRSTS | | GSGSGTDFTLTISSLQP | |
| | ESTAALGCLVKDYFPEPVTVSWNSGALTS | | EDFATYYCQQANSFPL | |
| | GVHTFPAVLQSSGLYSLSSVVTVPSSSLGT | | TFGGGTKVEIKRTVAA | |
| | KTYTCNVDHKPSNTKVDKRVSKYGPPCP | | PSVFIFPPSDEQLKSGT | |
| | SCPPEFLGGPSVFLFPPKPKDTLMISRTPE | | ASVVCLLNNFYPREA | |
| | VTCVVVDVSQEDPEVQFNWYVDGVEVH | | KVQWKVDNALQSGN | |
| | NAKTKPREEQFNSTYRVVSVLTVLHQDW | | SQESVTEQDSKDSTYS | |
| | LNGKEYKCKVSNKGLPSSIEKTISKAKQP | | LSSTLTLSKADYEKHK | |
| | REPQVYTLPPSQEEMTKNQVSLTCLVKGF | | VYACEVTHQGLSSPV | |
| | YPSDIAVEWESNGQPENNYKTTPPVLDSD | | TKSFNRGEC | |
| | GSFFLYSRLTVDKSRWQEGNVFSCSVMH | | | |
| | EALHNHYTQKSLSLSLGK | | | |
| 165 | QVQLVQSGAEVKKPGASVKVSCKASGYT | 1879 | EIVMTQSPATLSVSPG | 1967 |
| | FTGHYMHWVRQAPGQGLEWMGWISTD | | ERATLSCSASQSVGSS | |
| | NGNANYAQKFQGRVTMTRDTSTSTVYM | | YFAWYQQKPGQAPRL | |
| | ELSSLRSEDTAVYYCARDTADYYFDYWG | | LIYDVSTRATGIPARFS | |
| | QGTLVTVSSSTKGPSVFPLAPCSRSTSEST | | GSGSGTEFTLTISSLQS | |
| | AALGCLVKDYFPEPVTVSWNSGALTSGV | | EDFAVYYCQQYYSTP | |
| | HTFPAVLQSSGLYSLSSVVTVPSSSLGTKT | | LTFGPGTKVDIKRTVA | |
| | YTCNVDHKPSNTKVDKRVSKYGPPCPSC | | APSVFIFPPSDEQLKSG | |
| | PPEFLGGPSVFLFPPKPKDTLMISRTPEVT | | TASVVCLLNNFYPREA | |
| | CVVVDVSQEDPEVQFNWYVDGVEVHNA | | KVQWKVDNALQSGN | |
| | KTKPREEQFNSTYRVVSVLTVLHQDWLN | | SQESVTEQDSKDSTYS | |
| | GKEYKCKVSNKGLPSSIEKTISKAKQPREP | | LSSTLTLSKADYEKHK | |
| | QVYTLPPSQEEMTKNQVSLTCLVKGFYPS | | VYACEVTHQGLSSPV | |
| | DIAVEWESNGQPENNYKTTPPVLDSDGSF | | TKSFNRGEC | |
| | FLYSRLTVDKSRWQEGNVFSCSVMHEAL | | | |
| | HNHYTQKSLSLSLGK | | | |
| 166 | QVQLVQSGAEVKKPGSSVKVSCKASGGT | 1880 | DIQMTQSPSSLSASVG | 1968 |
| | FSRYPFSWVRQAPGQGLEWMGWMNPNN | | DRVTITCQASQDISNY | |
| | GDTGYAQKFQGRVTITADESTSTAYMEL | | LNWYQQKPGKAPKLL | |
| | SSLRSEDTAVYYCARGDYPYMDVWGKG | | IYDASNLETGVPSRFS | |
| | TTVTVSSSTKGPSVFPLAPCSRSTSESTAA | | GSGSGTDFTLTISSLQP | |
| | LGCLVKDYFPEPVTVSWNSGALTSGVHT | | EDFATYYCQQSYSIPY | |
| | FPAVLQSSGLYSLSSVVTVPSSSLGTKTYT | | TFGQGTKLEIKRTVAA | |
| | CNVDHKPSNTKVDKRVSKYGPPCPSCPPE | | PSVFIFPPSDEQLKSGT | |
| | FLGGPSVFLFPPKPKDTLMISRTPEVTCVV | | ASVVCLLNNFYPREA | |
| | VDVSQEDPEVQFNWYVDGVEVHNAKTK | | KVQWKVDNALQSGN | |
| | PREEQFNSTYRVVSVLTVLHQDWLNGKE | | SQESVTEQDSKDSTYS | |
| | YKCKVSNKGLPSSIEKTISKAKQPREPQV | | LSSTLTLSKADYEKHK | |
| | YTLPPSQEEMTKNQVSLTCLVKGFYPSDI | | VYACEVTHQGLSSPV | |
| | AVEWESNGQPENNYKTTPPVLDSDGSFFL | | TKSFNRGEC | |
| | YSRLTVDKSRWQEGNVFSCSVMHEALHN | | | |
| | HYTQKSLSLSLGK | | | |
| 167 | QVQLVQSGAEVKKPGASVKVSCKASGYT | 1881 | DIQMTQSPSSLSASVG | 1969 |
| | FTSDYMHWVRQAPGQGLEWMGWMNPN | | DRVTITCRASQGIRND | |
| | SGGTNYAQKFQGRVTMTRDTSTSTVYME | | LGWYQQKPGKAPKLL | |
| | LSSLRSEDTAVYYCARDYITGPSDWGQG | | IYAASSLQPGVPSRFS | |
| | TLVTVSSSTKGPSVFPLAPCSRSTSESTAA | | GSGSGTDFTLTISSLQP | |
| | LGCLVKDYFPEPVTVSWNSGALTSGVHT | | EDFATYYCLQTNSFP | |
| | FPAVLQSSGLYSLSSVVTVPSSSLGTKTYT | | WTFGQGTKLEIKRTV | |
| | CNVDHKPSNTKVDKRVSKYGPPCPSCPPE | | AAPSVFIFPPSDEQLKS | |
| | FLGGPSVFLFPPKPKDTLMISRTPEVTCVV | | GTASVVCLLNNFYPRE | |
| | VDVSQEDPEVQFNWYVDGVEVHNAKTK | | AKVQWKVDNALQSG | |
| | PREEQFNSTYRVVSVLTVLHQDWLNGKE | | NSQESVTEQDSKDSTY | |
| | YKCKVSNKGLPSSIEKTISKAKQPREPQV | | SLSSTLTLSKADYEKH | |
| | YTLPPSQEEMTKNQVSLTCLVKGFYPSDI | | KVYACEVTHQGLSSP | |
| | AVEWESNGQPENNYKTTPPVLDSDGSFFL | | VTKSFNRGEC | |
| | YSRLTVDKSRWQEGNVFSCSVMHEALHN | | | |
| | HYTQKSLSLSLGK | | | |
| 168 | QVQLVQSGAEVKKPGASVKVSCKASGFT | 1882 | DIQMTQSPSSLSASVG | 1970 |
| | FTSYYMHWVRQAPGQGLEWMGWMNPN | | DRVTITCRASQSISSW | |
| | SGNTGYAQRFQGRVTMTRDTSTSTVYME | | LAWYQQKPGKAPKLL | |
| | LSSLRSEDTAVYYCARGHSRTDYGMDV | | IYDTSSLQSGVPSRFSG | |
| | WGQGTTVTVSSSTKGPSVFPLAPCSRSTS | | SGSGTDFTLTISSLQPE | |
| | ESTAALGCLVKDYFPEPVTVSWNSGALTS | | DFATYYCQQGYSTPL | |
| | GVHTFPAVLQSSGLYSLSSVVTVPSSSLGT | | TFGQGTKVEIKRTVAA | |
| | KTYTCNVDHKPSNTKVDKRVSKYGPPCP | | PSVFIFPPSDEQLKSGT | |
| | SCPPEFLGGPSVFLFPPKPKDTLMISRTPE | | ASVVCLLNNFYPREA | |
| | VTCVVVDVSQEDPEVQFNWYVDGVEVH | | KVQWKVDNALQSGN | |
| | NAKTKPREEQFNSTYRVVSVLTVLHQDW | | SQESVTEQDSKDSTYS | |
| | LNGKEYKCKVSNKGLPSSIEKTISKAKQP | | LSSTLTLSKADYEKHK | |
| | REPQVYTLPPSQEEMTKNQVSLTCLVKGF | | VYACEVTHQGLSSPV | |
| | YPSDIAVEWESNGQPENNYKTTPPVLDSD | | TKSFNRGEC | |
| | GSFFLYSRLTVDKSRWQEGNVFSCSVMH | | | |
| | EALHNHYTQKSLSLSLGK | | | |
| 169 | EVQLLESGGGLVQPGGSLRLSCAASGFTF | 1883 | DIQMTQSPSSLSASVG | 1971 |
| | SDHYMSWVRQAPGKGLEWVSIIYPDGKT | | DRVTITCQASQDISNY | |
| | YYADSVKGRFTISRDNSKNTLYLQMNSL | | LNWYQQKPGKAPKLL | |
| | RAEDTAVYYCAREGSYGDYDGMDVWG | | IYGASTLQSGVPSRFS | |
| | QGTTVTVSSSTKGPSVFPLAPCSRSTSEST | | GSGSGTDFTLTISSLQP | |
| | AALGCLVKDYFPEPVTVSWNSGALTSGV | | EDFATYYCQQSYSTP | |
| | HTFPAVLQSSGLYSLSSVVTVPSSSLGTKT | | WTFGQGTKLEIKRTV | |
| | YTCNVDHKPSNTKVDKRVSKYGPPCPSC | | AAPSVFIFPPSDEQLKS | |
| | PPEFLGGPSVFLFPPKPKDTLMISRTPEVT | | GTASVVCLLNNFYPRE | |
| | CVVVDVSQEDPEVQFNWYVDGVEVHNA | | AKVQWKVDNALQSG | |
| | KTKPREEQFNSTYRVVSVLTVLHQDWLN | | NSQESVTEQDSKDSTY | |
| | GKEYKCKVSNKGLPSSIEKTISKAKQPREP | | SLSSTLTLSKADYEKH | |
| | QVYTLPPSQEEMTKNQVSLTCLVKGFYPS | | KVYACEVTHQGLSSP | |
| | DIAVEWESNGQPENNYKTTPPVLDSDGSF | | VTKSFNRGEC | |
| | FLYSRLTVDKSRWQEGNVFSCSVMHEAL | | | |
| | HNHYTQKSLSLSLGK | | | |
| 170 | QVQLVQSGAEVKKPGSSVKVSCKASGGT | 1884 | EIVMTQSPATLSVSPG | 1972 |
| | FSNYDISWVRQAPGQGLEWMGGIIPIFGT | | ERATLSCRASQSVSSY | |
| | ANYAQKFQGRVTITADESTSTAYMELSSL | | LAWYQQKPGQAPRLL | |
| | RSEDTAVYYCAREAEEGGWFDPWGQGT | | IYGASTRATGIPARFSG | |
| | LVTVSSSTKGPSVFPLAPCSRSTSESTAAL | | SGSGTEFTLTISSLQSE | |
| | GCLVKDYFPEPVTVSWNSGALTSGVHTF | | DFAVYYCQQYAFSPIT | |
| | PAVLQSSGLYSLSSVVTVPSSSLGTKTYTC | | FGQGTKLEIKRTVAAP | |
| | NVDHKPSNTKVDKRVSKYGPPCPSCPPEF | | SVFIFPPSDEQLKSGTA | |
| | LGGPSVFLFPPKPKDTLMISRTPEVTCVVV | | SVVCLLNNFYPREAK | |
| | DVSQEDPEVQFNWYVDGVEVHNAKTKP | | VQWKVDNALQSGNS | |
| | REEQFNSTYRVVSVLTVLHQDWLNGKEY | | QESVTEQDSKDSTYSL | |
| | KCKVSNKGLPSSIEKTISKAKQPREPQVYT | | SSTLTLSKADYEKHKV | |
| | LPPSQEEMTKNQVSLTCLVKGFYPSDIAV | | YACEVTHQGLSSPVT | |
| | EWESNGQPENNYKTTPPVLDSDGSFFLYS | | KSFNRGEC | |
| | RLTVDKSRWQEGNVFSCSVMHEALHNH | | | |
| | YTQKSLSLSLGK | | | |
| 171 | QVQLVQSGAEVKKPGASVKVSCKASGYT | 1885 | DIQMTQSPSSLSASVG | 1973 |
| | FTDYYMHWVRQAPGQGLEWMGWMNPN | | DRVTITCRVSQGISSYL | |
| | SGYTAYAQKFQGRVTMTRDTSTSTVYME | | NWYQQKPGKAPKLLI | |
| | LSSLRSEDTAVYYCAKDTPGSGWSSGMD | | YDASNLETGVPSRFSG | |
| | VWGQGTTVTVSSSTKGPSVFPLAPCSRST | | SGSGTDFTLTISSLQPE | |
| | SESTAALGCLVKDYFPEPVTVSWNSGALT | | DFATYYCQQSYSTPLT | |
| | SGVHTFPAVLQSSGLYSLSSVVTVPSSSLG | | FGGGTKVEIKRTVAAP | |
| | TKTYTCNVDHKPSNTKVDKRVSKYGPPC | | SVFIFPPSDEQLKSGTA | |
| | PSCPPEFLGGPSVFLFPPKPKDTLMISRTPE | | SVVCLLNNFYPREAK | |
| | VTCVVVDVSQEDPEVQFNWYVDGVEVH | | VQWKVDNALQSGNS | |
| | NAKTKPREEQFNSTYRVVSVLTVLHQDW | | QESVTEQDSKDSTYSL | |
| | LNGKEYKCKVSNKGLPSSIEKTISKAKQP | | SSTLTLSKADYEKHKV | |
| | REPQVYTLPPSQEEMTKNQVSLTCLVKGF | | YACEVTHQGLSSPVT | |
| | YPSDIAVEWESNGQPENNYKTTPPVLDSD | | KSFNRGEC | |
| | GSFFLYSRLTVDKSRWQEGNVFSCSVMH | | | |
| | EALHNHYTQKSLSLSLGK | | | |
| 172 | QVQLVQSGAEVKKPGASVKVSCKASGGT | 1886 | DIQMTQSPSSLSASVG | 1974 |
| | FSNYAISWVRQAPGQGLEWMGWINPNSG | | DRVTITCRASQSISSW | |
| | GTNYAQKFQGRVTMTRDTSTSTVYMELS | | LAWYQQKPGKAPKLL | |
| | SLRSEDTAVYYCARVGYYDSSGGGMDV | | IYDASNLETGVPSRFS | |
| | WGQGTTVTVSSSTKGPSVFPLAPCSRSTS | | GSGSGTDFTLTISSLQP | |
| | ESTAALGCLVKDYFPEPVTVSWNSGALTS | | EDFATYYCLQTHSFPL | |
| | GVHTFPAVLQSSGLYSLSSVVTVPSSSLGT | | TFGPGTKVDIKRTVAA | |
| | KTYTCNVDHKPSNTKVDKRVSKYGPPCP | | PSVFIFPPSDEQLKSGT | |
| | SCPPEFLGGPSVFLFPPKPKDTLMISRTPE | | ASVVCLLNNFYPREA | |
| | VTCVVVDVSQEDPEVQFNWYVDGVEVH | | KVQWKVDNALQSGN | |
| | NAKTKPREEQFNSTYRVVSVLTVLHQDW | | SQESVTEQDSKDSTYS | |
| | LNGKEYKCKVSNKGLPSSIEKTISKAKQP | | LSSTLTLSKADYEKHK | |
| | REPQVYTLPPSQEEMTKNQVSLTCLVKGF | | VYACEVTHQGLSSPV | |
| | YPSDIAVEWESNGQPENNYKTTPPVLDSD | | TKSFNRGEC | |
| | GSFFLYSRLTVDKSRWQEGNVFSCSVMH | | | |
| | EALHNHYTQKSLSLSLGK | | | |
| 173 | QVQLVQSGAEVKKPGASVKVSCKASGYT | 1887 | DIQMTQSPSSLSASVG | 1975 |
| | FTGYYMHWVRQAPGQGLEWMGIINPIGG | | DRVTITCRASQSVSNW | |
| | LTTYAQKFQGRVTMTRDTSTSTVYMELS | | LAWYQQKPGKAPKLL | |
| | SLRSEDTAVYYCASGAYGDYVDWYFDL | | IYDASNLQTGVPSRFS | |
| | WGRGTLVTVSSSTKGPSVFPLAPCSRSTS | | GSGSGTDFTLTISSLQP | |
| | ESTAALGCLVKDYFPEPVTVSWNSGALTS | | EDFATYYCQQANSFPL | |
| | GVHTFPAVLQSSGLYSLSSVVTVPSSSLGT | | TFGGGTKLEIKRTVAA | |
| | KTYTCNVDHKPSNTKVDKRVSKYGPPCP | | PSVFIFPPSDEQLKSGT | |
| | SCPPEFLGGPSVFLFPPKPKDTLMISRTPE | | ASVVCLLNNFYPREA | |
| | VTCVVVDVSQEDPEVQFNWYVDGVEVH | | KVQWKVDNALQSGN | |
| | NAKTKPREEQFNSTYRVVSVLTVLHQDW | | SQESVTEQDSKDSTYS | |
| | LNGKEYKCKVSNKGLPSSIEKTISKAKQP | | LSSTLTLSKADYEKHK | |
| | REPQVYTLPPSQEEMTKNQVSLTCLVKGF | | VYACEVTHQGLSSPV | |
| | YPSDIAVEWESNGQPENNYKTTPPVLDSD | | TKSFNRGEC | |
| | GSFFLYSRLTVDKSRWQEGNVFSCSVMH | | | |
| | EALHNHYTQKSLSLSLGK | | | |
| 174 | QVQLVQSGAEVKKPGASVKVSCKASGYT | 1888 | DIVMTQSPLSLPVTPG | 1976 |
| | FTTYGISWVRQAPGQGLEWMGWINPNSG | | EPASISCRSSRSLLHSN | |
| | DTNYAQKFQGRVTMTRDTSTSTVYMELS | | GYNYLDWYLQKPGQS | |
| | SLRSEDTAVYYCARLTTATDSFDLWGRG | | PQLLIYLGSYRASGVP | |
| | TLVTVSSSTKGPSVFPLAPCSRSTSESTAA | | DRFSGSGSGTDFTLKIS | |
| | LGCLVKDYFPEPVTVSWNSGALTSGVHT | | RVEAEDVGVYYCMQ | |
| | FPAVLQSSGLYSLSSVVTVPSSSLGTKTYT | | GTHWPPTFGQGTKLEI | |
| | CNVDHKPSNTKVDKRVSKYGPPCPSCPPE | | KRTVAAPSVFIFPPSDE | |
| | FLGGPSVFLFPPKPKDTLMISRTPEVTCVV | | QLKSGTASVVCLLNN | |
| | VDVSQEDPEVQFNWYVDGVEVHNAKTK | | FYPREAKVQWKVDN | |
| | PREEQFNSTYRVVSVLTVLHQDWLNGKE | | ALQSGNSQESVTEQDS | |
| | YKCKVSNKGLPSSIEKTISKAKQPREPQV | | KDSTYSLSSTLTLSKA | |
| | YTLPPSQEEMTKNQVSLTCLVKGFYPSDI | | DYEKHKVYACEVTHQ | |
| | AVEWESNGQPENNYKTTPPVLDSDGSFFL | | GLSSPVTKSFNRGEC | |
| | YSRLTVDKSRWQEGNVFSCSVMHEALHN | | | |
| | HYTQKSLSLSLGK | | | |
| 175 | QVQLVQSGAEVKKPGASVKVSCKASGYS | 1889 | DIQMTQSPSSLSASVG | 1977 |
| | FTNYYIHWVRQAPGQGLEWMGWMNPY | | DRVTITCRASQSISSYL | |
| | TGQTGYAQKFQGRVTMTRDTSTSTVYME | | NWYQQKPGKAPKLLI | |
| | LSSLRSEDTAVYYCTTDEETMDFHLWGR | | YDASNLETGVPSRFSG | |
| | GTLVTVSSSTKGPSVFPLAPCSRSTSESTA | | SGSGTDFTLTISSLQPE | |
| | ALGCLVKDYFPEPVTVSWNSGALTSGVH | | DFATYYCQQANTFPIT | |
| | TFPAVLQSSGLYSLSSVVTVPSSSLGTKTY | | FGQGTRLEIKRTVAAP | |
| | TCNVDHKPSNTKVDKRVSKYGPPCPSCPP | | SVFIFPPSDEQLKSGTA | |
| | EFLGGPSVFLFPPKPKDTLMISRTPEVTCV | | SVVCLLNNFYPREAK | |
| | VVDVSQEDPEVQFNWYVDGVEVHNAKT | | VQWKVDNALQSGNS | |
| | KPREEQFNSTYRVVSVLTVLHQDWLNGK | | QESVTEQDSKDSTYSL | |
| | EYKCKVSNKGLPSSIEKTISKAKQPREPQV | | SSTLTLSKADYEKHKV | |
| | YTLPPSQEEMTKNQVSLTCLVKGFYPSDI | | YACEVTHQGLSSPVT | |
| | AVEWESNGQPENNYKTTPPVLDSDGSFFL | | KSFNRGEC | |
| | YSRLTVDKSRWQEGNVFSCSVMHEALHN | | | |
| | HYTQKSLSLSLGK | | | |
| 176 | QVQLVQSGAEVKKPGASVKVSCKASGYT | 1890 | DIVMTQSPLSLPVTPG | 1978 |
| | FTGYHIHWVRQAPGQGLEWMGRINPNSG | | EPASISCRSSRSLLHSN | |
| | GTDYAQKFQGRVTMTRDTSTSTVYMELS | | GYNYLDWYLQKPGQS | |
| | SLRSEDTAVYYCARETYSGSYEESFDYW | | PQLLIYLGSDRASGVP | |
| | GQGTLVTVSSSTKGPSVFPLAPCSRSTSES | | DRFSGSGSGTDFTLKIS | |
| | TAALGCLVKDYFPEPVTVSWNSGALTSG | | RVEAEDVGVYYCMQ | |
| | VHTFPAVLQSSGLYSLSSVVTVPSSSLGTK | | GTHWPPTFGQGTKVEI | |
| | TYTCNVDHKPSNTKVDKRVSKYGPPCPS | | KRTVAAPSVFIFPPSDE | |
| | CPPEFLGGPSVFLFPPKPKDTLMISRTPEV | | QLKSGTASVVCLLNN | |
| | TCVVVDVSQEDPEVQFNWYVDGVEVHN | | FYPREAKVQWKVDN | |
| | AKTKPREEQFNSTYRVVSVLTVLHQDWL | | ALQSGNSQESVTEQDS | |
| | NGKEYKCKVSNKGLPSSIEKTISKAKQPR | | KDSTYSLSSTLTLSKA | |
| | EPQVYTLPPSQEEMTKNQVSLTCLVKGFY | | DYEKHKVYACEVTHQ | |
| | PSDIA VEWESNGQPENNYKTTPPVLDSDG | | GLSSPVTKSFNRGEC | |
| | SFFLYSRLTVDKSRWQEGNVFSCSVMHE | | | |
| | ALHNHYTQKSLSLSLGK | | | |

### Example 15: AML Cell Line Xenograft Model Using Antibody Drug Conjugate

Antibody drug conjugates may be generated by conjugating a biologically active compound to a variant specific antibody. Examples may include the conjugation of molecules such as saporin (a ribosome inactivating protein), MMAE, MMAF, DM1, or DM4 to an anti-CD33^{R69} antibody or anti-CD33^{G69} antibody, leading to cell death upon antigen binding and antibody mediated internalization of the drug.

Six to ten week old immunodeficient NOD.Cg-Prkdcscid Il2rgtm1Wjl Tg(CMV-IL3,CSF2,KITLG)1Eav/MloySzJ (NSG-SGM3) mice may be used in murine patient-derived xenograft experiments. Both male and female mice may be used in experiments and randomly assigned to a treatment group.

Target AML cell lines may be obtained from commercially vendors (ATCC). Target expression was confirmed by FACS analysis and target cell genotype obtained through DNA sequencing. Cells may be modified to express CBR-GFP (Click beetle luciferase and Green Fluorescent Protein).

Mice are engrafted with an appropriate amount, e.g., 1×10⁶ cells on day −7. On day 0 mice are treated with an appropriate amount of ADC (e.g., dosing ranging from 0.1 mg/kg to 5 mg/kg) on days 0, +7, and +14.

For example, a CD33^{R69} AML Cell line, KG1a, may be engrafted into mice and treated with either anti-CD33^{G69}-saporin or anti-CD33^{R69}-saporin, a positive control (anti-CD33-saporin) or a negative control (anti-CD33 and free saporin).

Tumor burden may be monitored by bioluminescent imaging (BLI) weekly. Mice will be monitored for survival Bone marrow may be extracted from mice and tumor burden assessed using FACS.

It is expected that anti-CD33-saporin (positive control) will kill CD33+ targets independent of the CD33 genotype (CD33^{R69} or CD33^{G69}), reduce tumor burden, and prolong survival. Anti-CD33^{R69}-saporin is expected to kill CD33^{R69} targets (example KG1a), reduce tumor burden, and prolong survival of mice. Anti-CD33^{G69}-saporin would not be expected to kill CD33^{R69} targets and would not offer a survival advantage or reduce tumor burden.

### Example 16: Clinical Applications

Several clinical applications of polymorphically selective treatment of subjects are given below. In the examples below, the polymorphic antigen may be, e.g., CD33, FLT3, or CLL-1; for illustrative purposes, CD33 will be used.

Scenario 1: No prior screening, screen patients upon relapse. In this scenario, a subject with cancer, e.g. MDS or AML, is conditioned and transplanted with HSC from a related or unrelated histocompatible donor, whether from a human leukocyte antigen (HLA)-identical sibling, a HLA-matched donor, a cord blood unit, or a haploidentical donor, screened for a low probability of allorejection and graft-versus-host disease (GvHD). Most HSCT recipients eventually relapse. If relapse occurs, the subject becomes eligible for therapy with polymorphically selective treatment such as CAR-bearing immune effector cells (e.g., TCR-deleted CAR-T or CAR-NK or CAR-iNKT), or NK cells in combination with an antibody that induces ADCC) that target an antigen expressed on the surface of the subject's malignant cells.

If the subject relapses post-transplant, the subject is then genotyped using either a protein- (e.g. FACS) or DNA- (PCR) based approach to ensure the HSC donor and patient express different variants of the target antigen, e.g., CD33. If the subject and donor do express different variants of target antigen, e.g. one expresses CD33^{R69} and the other expresses CD33^{G69}, the subject is eligible for polymorphic treatment. The subject is then conditioned (e.g., cyclophosphamide/fludarabine, 3 days) and treated with CAR-bearing immune effector cells (e.g., TCR-deleted CAR-T or CAR-NK or CAR-iNKT), or NK cells in combination with an antibody that induces ADCC targeting the patient specific target antigen. For example, the patient whose cells express CD33^{R69} and whose HSCT graft expresses may be treated with CD33^{G69} may be treated with TCR-deleted CD33^{R69}-CART, or CD33^{R69}-CAR-NK, or donor NK cells in combination with an anti-CD33^{R69} antibody that induces ADCC. The CD33^{R69}-selective therapy will kill the subject's cancerous cells and spare the CD33^{G69}-expressing HSCT cells. The reverse mismatched combination would also be effective. The subject may then be monitored and, optionally, retreated with one or more of these selective therapies.

Scenario 2: Prospective screening, screen patients upon relapse. In this scenario, HSCT donors are prospectively screened to assess the donor's expression of a polymorphic variant of a given target antigen, e.g., CD33, and identify a donor who expresses a different variant than the prospective recipient subject. This can be done by genotyping patient and donor using a (PCR) based genotyping approach. At this time, both HSC and immune effector cells (such as T cells, NK cells, and iNKT cells) may be harvested from the same donor and separated via leukapheresis. The HSC may be used for transplant into the target-mismatched recipient; and the immune effector cells may be transduced with a CAR that selectively binds the variant of the antigen (e.g., CD33^{R69} or CD33^{G69}) expressed by the recipient's, but not the donor's cells, or stored for later use if needed.

The subject is conditioned and transplanted with HSC from a target-mismatched donor, e.g. a donor who expresses CD33^{G69} and for a patient who expresses CD33^{R69}, or the reverse. The donor may be a related or unrelated histocompatible donor as above.

If relapse occurs, the subject is conditioned (e.g., cyclophosphamide/fludarabine, 3 days) and treated with polymorphically selective treatment such as CAR-bearing immune effector cells (e.g., CAR-T, TCR-deleted CAR-T, CAR-NK, or CAR-iNKT), or NK cells in combination with an antibody that induces ADCC) that target the variant of the antigen expressed on the surface of the subject's malignant cells and not the variant expressed on the surface of the donor's cells. For example, the patient whose cells express CD33^{R69} and whose HSCT graft expresses may be treated with CD33^{G69} may be treated with TCR-deleted CD33^{R69}-CART, or CD33^{R69}-CAR-NK, or donor NK cells in combination with an anti-CD33^{R69} antibody that induces ADCC. The CD33^{R69}-selective therapy will kill the subject's CD33^{R69}-expressing cancerous cells and spare the CD33^{G69}-expressing HSCT cells. The reverse mismatched combination would also be effective. The subject may then be monitored and, optionally, retreated with one or more of these selective therapies.

Scenario 3: Prospective screening, treat patients upon relapse. In this scenario, HSCT donors are prospectively screened to assess the donor's expression of a polymorphic variant of a given target antigen, e.g., CD33, and identify a donor who expresses a different variant than the prospective recipient subject. This can be done by genotyping patient and donor using a (PCR) based genotyping approach.

The subject is conditioned and transplanted with HSC from a target-mismatched donor, e.g. a donor who expresses CD33^{G69} and for a patient who expresses CD33^{R69}, or the reverse. The donor may be a related or unrelated histocompatible donor as above. If relapse occurs, the subject is conditioned (e.g., cyclophosphamide/fludarabine, 3 days) and treated with polymorphically selective treatment such as CAR-bearing immune effector cells (e.g., TCR-deleted CAR-T or CAR-NK or CAR-iNKT), or NK cells in combination with an antibody that induces ADCC, or an antibody-drug conjugate comprising an antibody that induces ADCC, that target the variant of the antigen expressed on the surface of the subject's malignant cells and not the variant expressed on the surface of the donor's cells. For example, the patient whose cells express CD33^{R69} and whose HSCT graft expresses may be treated with CD33^{G69} may be treated with TCR-deleted CD33^{R69}-CART, or CD33^{R69}-CAR-NK, or donor NK cells in combination with an anti-CD33^{R69} antibody that induces ADCC. The CD33^{R69}-selective therapy will kill the subject's CD33^{R69}-expressing cancerous cells and spare the CD33^{G69}-expressing HSCT cells. The reverse mismatched combination would also be effective. The subject may then be monitored and, optionally, retreated with one or more of these selective therapies.

Scenario 4: Prospective screening, treat at time of transplant. In this scenario, HSCT donors are prospectively screened to assess the donor's expression of a polymorphic variant of a given target antigen, e.g., CD33, and identify a donor who expresses a different variant than the prospective recipient subject. This can be done by genotyping patient and donor using a (PCR) based genotyping approach.

The subject is conditioned and transplanted with HSC from a target-mismatched donor, e.g. a donor who expresses CD33^{G69} and for a patient who expresses CD33^{R69}, or the reverse. The donor may be a related or unrelated histocompatible donor as above. The conditioning may be as for standard HSCT (fully myeloablative), or reduced intensity conditioning (RIC); or alternatively, could be a T cell depleted transplant.

Nearly concurrently with transplant—that is, within 1 day, 2 days, 3 days, or 10 days, etc. of HSCT, but in any event, not requiring relapse—the subject is treated with polymorphically selective treatment such as CAR-bearing immune effector cells (e.g., CAR-T, TCR-deleted CAR-T, CAR-NK, or CAR-iNKT), or NK cells in combination with an antibody that induces ADCC) that target the variant of the antigen expressed on the surface of the subject's malignant cells and not the variant expressed on the surface of the donor's cells. For example, the patient whose cells express CD33^{R69} and whose HSCT graft expresses may be treated with CD33^{G69} may be treated with TCR-deleted CD33^{R69}-CART, or CD33^{R69}-CAR-NK, or donor NK cells in combination with an anti-CD33^{R69} antibody that induces ADCC. The CD33^{R69}-selective therapy will kill the subject's CD33^{R69}-expressing cancerous cells and spare the CD33^{G69}-expressing HSCT cells. The reverse mismatched combination would also be effective. The subject may then be monitored and, optionally, retreated with one or more of these selective therapies.

The foregoing methods may be adapted to demonstrate the binding and polymorphic selectivity of other scFvs, antibodies, antibody-drug conjugates, and CARs against antigens such as cancer antigens. For example, the methods are expected to demonstrate anti-FLT3 scFvs that selectively bind either the T227 or M227 variants. The methods are also expected to demonstrate anti-CLL-1 scFvs that selectively bind either the K244 or Q244 variant.

### Example 17: Identification of Non-Selective Anti-Human CD33 scFv Clones

The methods above in Example 1 have been used to discover polymorphically non-selective anti-human CD33 scFv clones.

TABLE 16a
Sequences of Non-Selective Anti-CD33 Polypeptides (CDR Sequences)

| Poly- | | SEQ | | SEQ | | SEQ | | SEQ | | SEQ | | SEQ |
|---|---|---|---|---|---|---|---|---|---|---|---|---|
| peptide | | ID | | ID | | ID | | ID | | ID | | ID |
| No. | HCDR1 | NO | HCDR2 | NO | HCDR3 | NO | LCDR1 | NO | LCDR2 | NO | LCDR3 | NO |
| 89 | YTFTN | 705 | GWINP | 760 | CARDD | 815 | RASQG | 870 | SASNL | 925 | CQQSF | 980 |
| | YYMH | | NSGDT | | RIQLW | | ISNYLA | | QS | | STPFT | |
| | | | NYE | | VPLVF | | | | | | F | |
| | | | | | W | | | | | | | |
| 90 | FTFSNS | 706 | SYISGT | 761 | CAKDY | 816 | RASQSI | 871 | AASNL | 926 | CQQYG | 981 |
| | DMN | | GSTIY | | DSSYG | | YNYLN | | QS | | NAPLT | |
| | | | YA | | SGYYG | | | | | | F | |
| | | | | | MDVW | | | | | | | |
| 91 | GTFSS | 707 | GWMN | 762 | CARED | 817 | QASHD | 872 | DASNL | 927 | CQQAY | 982 |
| | YAIS | | PNSGN | | YYDSS | | INIHLN | | ET | | SLPWT | |
| | | | TGYA | | GNFDY | | | | | | F | |
| | | | | | W | | | | | | | |
| 92 | YTFTN | 708 | GIINPS | 763 | CASAE | 818 | RSSQS | 873 | AASSL | 928 | CMQAL | 983 |
| | HYMH | | GGSTS | | VGATH | | LLHSN | | QS | | HPPTF | |
| | | | YA | | YGMD | | GYNYL | | | | | |
| | | | | | VW | | D | | | | | |
| 93 | YSFTN | 709 | GWVN | 764 | CAKDA | 819 | RSSQS | 874 | AASSL | 929 | CMQAL | 984 |
| | YDIS | | PNSGN | | PYYYD | | LLHSN | | QS | | QTPLT | |
| | | | TGYA | | SSGYY | | GYNYL | | | | F | |
| | | | | | GVFDY | | D | | | | | |
| | | | | | W | | | | | | | |
| 94 | YTFTT | 710 | GWISG | 765 | CAKD | 820 | RSSQS | 875 | AASTL | 930 | CMQAL | 985 |
| | YDIN | | YNGNT | | MGYG | | LLHSN | | QS | | QIPIT | |
| | | | GYA | | DYPDA | | GYNYL | | | | F | |
| | | | | | FDIW | | D | | | | | |
| 95 | YSFTT | 711 | GRMNP | 766 | CARVV | 821 | RSSQS | 876 | LGSLR | 931 | CMQAL | 986 |
| | YDIN | | NSGNT | | HGMD | | LLHSN | | AP | | QTPWT | |
| | | | GYA | | VW | | GYNYL | | | | F | |
| | | | | | | | D | | | | | |
| 96 | GTFSS | 712 | GWMN | 767 | CAKDE | 822 | RASQSI | 877 | GVSTL | 932 | CQQSY | 987 |
| | YAIS | | PSSAN | | MELLT | | GSWLA | | HS | | STPPT | |
| | | | TGYA | | AFDIW | | | | | | F | |
| 97 | FTFRS | 713 | SVISGS | 768 | CARET | 823 | RASQSI | 878 | DASNL | 933 | CQQSSI | 988 |
| | YWMT | | GDNTY | | TWGM | | SSYLN | | ET | | IPLTF | |
| | | | YA | | DVW | | | | | | | |
| 98 | GSFSSS | 714 | GWMN | 769 | CARDR | 824 | RASQD | 879 | AASSL | 934 | CHQSY | 989 |
| | AIN | | PNSGN | | GIAVA | | IGSYLA | | QS | | STPFT | |
| | | | TGYA | | GASPY | | | | | | F | |
| | | | | | YYYG | | | | | | | |
| | | | | | MDVW | | | | | | | |
| 99 | YTFSD | 715 | GWMN | 770 | CARTH | 825 | RASQG | 880 | QASNK | 935 | CQQSY | 990 |
| | YHIH | | PNSGN | | SSGYY | | ISNNLN | | DT | | SSPPT | |
| | | | TGYA | | YWFDP | | | | | | F | |
| | | | | | W | | | | | | | |
| 100 | ATRSW | 716 | GIINPS | 771 | CAKEP | 826 | RASQSI | 881 | GASSL | 936 | CQQSY | 991 |
| | MH | | GDSTS | | YSSSP | | SSWLA | | QS | | TTPITF | |
| | | | YA | | YYFDY | | | | | | | |
| | | | | | W | | | | | | | |
| 101 | FTFSSY | 717 | SAISGD | 772 | CARDT | 827 | RASQN | 882 | AASTL | 937 | CQQYD | 992 |
| | GVH | | GGDTY | | WDYS | | INTFLN | | QS | | SFPLTF | |
| | | | YA | | NYGGI | | | | | | | |
| | | | | | DYW | | | | | | | |
| 102 | FTFSN | 718 | SGIGGS | 773 | CAREV | 828 | RSSQS | 883 | LGSNR | 938 | CMQAL | 993 |
| | | | GGTIY | | AAPLH | | LLHSN | | AS | | ETPITF | |
| | AWMS | | YA | | PFGYY | | GYNYL | | | | | |
| | | | | | YYMD | | D | | | | | |
| | | | | | VW | | | | | | | |
| 103 | YTFTG | 719 | GWMN | 774 | CATTR | 829 | RSSQS | 884 | LGSNR | 939 | CMQAT | 994 |
| | YYMH | | PDSGD | | QPHYG | | LLHSN | | AS | | HWPTF | |
| | | | TNYA | | MDVW | | GYNYL | | | | | |
| | | | | | | | D | | | | | |
| 104 | FTFSSS | 720 | AVISY | 775 | CARLT | 830 | RASQG | 885 | AASSL | 940 | CQQSY | 995 |
| | WMH | | DGSEE | | DYGDY | | ISSYLA | | QS | | SIPPTF | |
| | | | YYA | | VLGRY | | | | | | | |
| | | | | | LSDW | | | | | | | |
| 105 | FTFNN | 721 | AVISY | 776 | CARM | 831 | RASQSI | 886 | AASTL | 941 | CQQTY | 996 |
| | AWMT | | DGSNK | | AVAGK | | YSWLA | | QS | | STPVTF | |
| | | | YYA | | GAFDI | | | | | | | |
| | | | | | W | | | | | | | |
| 106 | YTFTG | 722 | GRIKP | 777 | CARGA | 832 | RASQSI | 887 | AASSL | 942 | CQQYG | 997 |
| | YYMH | | NSGGT | | YSGSY | | SWFLN | | QN | | SFPPTF | |
| | | | DYA | | YGPIE | | | | | | | |
| | | | | | YFQH | | | | | | | |
| | | | | | W | | | | | | | |
| 107 | YTFTD | 723 | GGIIPIF | 778 | CAREP | 833 | QASQD | 888 | AASTL | 943 | CQQSY | 998 |
| | YYIH | | GTANY | | LWFGE | | ISNYLN | | QS | | SSPPTF | |
| | | | A | | SSPHD | | | | | | | |
| | | | | | YYGM | | | | | | | |
| | | | | | DVW | | | | | | | |
| 108 | YTFTN | 724 | GWMN | 779 | CARG | 834 | RSSQS | 889 | WASTR | 944 | CQQYY | 999 |
| | YDIN | | PNSGN | | WGHG | | LLYSS | | ES | | SNPLTF | |
| | | | TGLV | | YGDYK | | NNLNY | | | | | |
| | | | | | FDYW | | LA | | | | | |
| 109 | YTFTT | 725 | GWMN | 780 | CAREG | 835 | RASQSI | 890 | GASTR | 945 | CQQYE | 1000 |
| | YGIS | | PNSGN | | GDGDY | | SSSSLA | | AT | | TAPYT | |
| | | | TGYA | | PDYW | | | | | | F | |
| 110 | YTFTD | 726 | GWMN | 781 | CARDF | 836 | RASQRI | 891 | AASSL | 946 | CQQSY | 1001 |
| | YYVH | | PNSGN | | IWVEG | | GNWL | | QS | | STPLTF | |
| | | | TGYA | | YLASP | | A | | | | | |
| | | | | | PPRFD | | | | | | | |
| | | | | | YW | | | | | | | |
| 111 | GTFTS | 727 | GWINP | 782 | CANEQ | 837 | RASQS | 892 | GASTR | 947 | CQQYY | 1002 |
| | YGIS | | NTGVT | | GGFDY | | VAGSY | | AT | | STPLTF | |
| | | | NYA | | W | | LA | | | | | |
| 112 | STLTG | 728 | GGIIPF | 783 | CARGG | 838 | RSSESI | 893 | SASTL | 948 | CQQSY | 1003 |
| | YDIH | | LGTAS | | GSGYD | | SSWLA | | QS | | STPVTF | |
| | | | YA | | LDYW | | | | | | | |
| 113 | GTFSS | 729 | GGLIP | 784 | CATGL | 839 | RASQG | 894 | AASTL | 949 | CQQTY | 1004 |
| | YDIN | | VFGTT | | GVTTS | | IRNDIG | | QS | | MMPYT | |
| | | | HYA | | NYYYG | | | | | | F | |
| | | | | | MDVW | | | | | | | |
| 114 | GTFSK | 730 | GWMN | 785 | CARDQ | 840 | RASQSI | 895 | KASSL | 950 | CQQSY | 1005 |
| | YAIS | | PNSGN | | GLTGY | | GNWL | | ES | | NTPPTF | |
| | | | TGYA | | FDLW | | A | | | | | |
| 115 | YTFTG | 731 | GIISPS | 786 | CAREG | 841 | RSSQS | 896 | LGSNR | 951 | CMQAL | 1006 |
| | YYMH | | GGSPT | | NGGM | | LLHSN | | AS | | QTPYT | |
| | | | YA | | DVW | | GYNYL | | | | F | |
| | | | | | | | D | | | | | |
| 116 | FTFSN | 732 | SAISGS | 787 | CAREG | 842 | RASQSI | 897 | ATSRL | 952 | CQQGF | 1007 |
| | YAMA | | GGGTY | | GYDPD | | SSYLN | | QS | | NFPPTF | |
| | | | YA | | YYYYG | | | | | | | |
| | | | | | MDVW | | | | | | | |
| 117 | FTFGD | 733 | AGISY | 788 | CARDR | 843 | RASQTI | 898 | DASSL | 953 | CQQSY | 1008 |
| | YPMS | | DGLNE | | DSGPS | | GTWLA | | ES | | STPPTF | |
| | | | HYA | | GFQH | | | | | | | |
| | | | | | W | | | | | | | |
| 118 | FTISNA | 734 | AHIWN | 789 | CARDG | 844 | RSSQS | 899 | AASSL | 954 | CMQGL | 1009 |
| | WMS | | DGSQK | | ALGVG | | LLHSN | | QS | | QTPHT | |
| | | | YYA | | PDDY | | GYNYL | | | | F | |
| | | | | | W | | D | | | | | |
| 119 | NTLTN | 735 | GWMN | 790 | CARAG | 845 | RSSQS | 900 | MGSNR | 955 | CMQAL | 1010 |
| | DHIH | | PNSGD | | VDTA | | LLHSN | | AS | | ETPTF | |
| | | | TGYA | | MVTY | | GYNYL | | | | | |
| | | | | | YYYG | | D | | | | | |
| | | | | | MDVW | | | | | | | |
| 120 | YTFTT | 736 | GWMN | 791 | CARGH | 846 | RASQSI | 901 | AISTLQ | 956 | CQQSY | 1011 |
| | YYMH | | PNSGN | | KVDSG | | GTYLH | | N | | SPPLTF | |
| | | | TGYA | | YDPYG | | | | | | | |
| | | | | | MDVW | | | | | | | |
| 121 | DSFTD | 737 | GWMN | 792 | CARDR | 847 | RASQN | 902 | AASTL | 957 | CQQSY | 1012 |
| | YYIH | | PNSGN | | EYSSSS | | IGNWL | | QS | | NSITF | |
| | | | TGYA | | RYFDL | | A | | | | | |
| | | | | | W | | | | | | | |
| 122 | YTFTD | 738 | GTINPS | 793 | CAKEE | 848 | RASQSI | 903 | AASSL | 958 | CQQSY | 1013 |
| | YWLH | | GGSTS | | EGFWS | | SSYLN | | QS | | STPLTF | |
| | | | YS | | GYAFD | | | | | | | |
| | | | | | YW | | | | | | | |
| 123 | FILGNA | 739 | ASVSG | 794 | CARDT | 849 | QASQD | 904 | AASNL | 959 | CQQTY | 1014 |
| | WMH | | DGSDE | | HDYGD | | INNYL | | QS | | SFPLTF | |
| | | | NYA | | YAPFD | | N | | | | | |
| | | | | | YW | | | | | | | |
| 124 | FTFSSY | 740 | AVIWY | 795 | CVRDG | 850 | RASQS | 905 | GASTR | 960 | CQQYY | 1015 |
| | WMH | | DGSNK | | ARSGM | | VSTYV | | AT | | DTPLTF | |
| | | | YYA | | DVW | | A | | | | | |
| 125 | YSFTT | 741 | GWMN | 796 | CATDH | 851 | QASQD | 906 | AASTL | 96 | CQQYS | 1016 |
| | YDIH | | PNSGN | | WVLG | | ISNYLN | | QS | | YLPVT | |
| | | | TGYA | | GFDY | | | | | | F | |
| | | | | | W | | | | | | | |
| 126 | FTFTTY | 742 | AGINW | 797 | CAKDL | 852 | RASQSI | 907 | AASSL | 962 | CQQSD | 1017 |
| | DMH | | NSVIID | | LYYYD | | STWLA | | QS | | TLPLTF | |
| | | | YA | | SIGAFD | | | | | | | |
| | | | | | IW | | | | | | | |
| 127 | YTFTN | 743 | GMINP | 798 | CARGR | 853 | QASQD | 908 | GASTL | 963 | CQQSY | 1018 |
| | HHMH | | SGGST | | PVDIV | | IRNFLN | | HS | | STPLTF | |
| | | | SYA | | ATYYF | | | | | | | |
| | | | | | DYW | | | | | | | |
| 128 | YTFTN | 744 | GWTNP | 799 | CAKEG | 854 | RASQG | 909 | AASSL | 964 | CQQSY | 1019 |
| | YYIH | | INGDT | | QLAW | | ISSALA | | QS | | STPLTF | |
| | | | GSA | | ADYYY | | | | | | | |
| | | | | | YMDV | | | | | | | |
| | | | | | W | | | | | | | |
| 129 | FSLRN | 745 | SGISGS | 800 | CARDY | 855 | RASQSI | 910 | AASSL | 965 | CQQSY | 1020 |
| | YWMH | | GGSTY | | TGVVD | | SSYLN | | QS | | STPLTF | |
| | | | YA | | YW | | | | | | | |
| 130 | GTFSN | 746 | AWMN | 801 | CARDG | 856 | RASQSI | 911 | GATRL | 966 | CQQSY | 1021 |
| | YAIS | | PNSGN | | FIGFGE | | GTWLA | | LS | | STPPTF | |
| | | | TGYA | | LFSAF | | | | | | | |
| | | | | | DIW | | | | | | | |
| 131 | GTFSN | 747 | GWINP | 802 | CARDS | 857 | QASQD | 912 | GASTL | 967 | CQQAY | 1022 |
| | YAIN | | NSGGT | | SLALS | | ISDHLN | | QS | | SFPWT | |
| | | | DSA | | YGGNS | | | | | | F | |
| | | | | | EYYYG | | | | | | | |
| | | | | | MDVW | | | | | | | |
| 132 | FTFNN | 748 | SAISGS | 803 | CAREY | 858 | RASQS | 913 | GASTR | 968 | CQQYG | 1023 |
| | YGMH | | GGSTY | | MQQPH | | VNSYL | | AT | | SSPLSF | |
| | | | YA | | GGMD | | A | | | | | |
| | | | | | VW | | | | | | | |
| 133 | FTFSSS | 749 | SAISSS | 804 | CAKFS | 859 | RASQG | 914 | TASSL | 969 | CQQYD | 1024 |
| | WMH | | GDATY | | DGGAG | | ISSYLA | | QS | | NLPITF | |
| | | | YA | | DSDY | | | | | | | |
| | | | | | W | | | | | | | |
| 134 | YTFDS | 750 | GMINP | 805 | CAKEG | 860 | RASQSI | 915 | AASSL | 970 | CQQSY | 1025 |
| | YLLH | | SGAGT | | SIAAG | | DSWLA | | QS | | TTPITF | |
| | | | TYA | | YYFDS | | | | | | | |
| | | | | | W | | | | | | | |
| 135 | YSFTT | 751 | GWINP | 806 | CASDL | 861 | KSSQS | 916 | WASTR | 97 | CQQYY | 1026 |
| | | | NSGNA | | AGYSS | | VLYGS | | | | | |
| | YGIT | | GYA | | GYFDL | | NNKNY | | ES | | STPLTF | |
| | | | | | W | | LA | | | | | |
| 136 | DTLTN | 752 | GWMN | 807 | CARDP | 862 | RASQRI | 917 | AASSL | 972 | CQQSY | 1027 |
| | HFVH | | PNSGN | | QMGA | | GNWL | | QS | | SPPLTF | |
| | | | TGYA | | VAGGF | | A | | | | | |
| | | | | | DYW | | | | | | | |
| 137 | YTFTD | 753 | GMVNP | 808 | CAKDS | 863 | RASQG | 918 | DASNL | 973 | CQQSY | 1028 |
| | YYIH | | SGGSA | | AWQEP | | ISSYLA | | DT | | STPLTF | |
| | | | NYA | | YYFDY | | | | | | | |
| | | | | | W | | | | | | | |
| 138 | YTFSS | 754 | GVINP | 809 | CARDE | 864 | RASQS | 919 | GASTR | 974 | CQQSH | 1029 |
| | YDMH | | GGGYT | | GWELL | | VGSNL | | AT | | SLPPTF | |
| | | | NYA | | LDYW | | A | | | | | |
| 139 | YTLSD | 755 | GWMN | 810 | CERDQ | 865 | RASQG | 920 | AASSL | 975 | CQQSY | 1030 |
| | HDIN | | PSTGN | | LRFGA | | IRNYL | | QS | | SIPLTF | |
| | | | TGYA | | WFDP | | A | | | | | |
| | | | | | W | | | | | | | |
| 140 | ITVSSS | 756 | SAIGT | 811 | CARDQ | 866 | RASQSI | 921 | DASTL | 976 | CQQSY | 1031 |
| | WMH | | GGGTH | | GGQID | | SSWLA | | QS | | SIPLTF | |
| | | | YA | | HW | | | | | | | |
| 141 | GTFSS | 757 | GVISPN | 812 | CARDR | 867 | RASQSI | 922 | AASSL | 977 | CQQSY | 1032 |
| | YAIS | | GDTTV | | GVAHS | | SSYLN | | HS | | SPPITF | |
| | | | YA | | YYYG | | | | | | | |
| | | | | | MDVW | | | | | | | |
| 142 | FTFSSY | 758 | AVISY | 813 | CARGL | 868 | RASQSI | 923 | AASSL | 978 | CQQSY | 1033 |
| | WMH | | DGSDK | | GGTTG | | SSYLN | | QS | | STPLTF | |
| | | | YYA | | TADFD | | | | | | | |
| | | | | | YW | | | | | | | |
| 143 | YTFTG | 759 | GWMN | 814 | CARDS | 869 | RASQSI | 924 | AASSL | 979 | CQQSY | 1034 |
| | YYMH | | ANSGN | | SSWLS | | GTYLS | | QS | | SSPITF | |
| | | | TGFA | | GGGW | | | | | | | |
| | | | | | FDPW | | | | | | | |
| 191 | GTFSS | 1979 | GWMN | 1982 | CARED | 1985 | QASHD | 1988 | DASNL | 1991 | CQQAY | 1994 |
| | YAIS | | PNSGN | | YYDSS | | INIHLN | | ET | | SLPWT | |
| | | | TGYA | | GNFDY | | | | | | F | |
| | | | | | W | | | | | | | |
| 192 | YTFTN | 1980 | GWINP | 1983 | CARDD | 1986 | RASQG | 1989 | SASNL | 1992 | CQQSF | 1995 |
| | YYMH | | NSGDT | | RIQLW | | ISNYLA | | QS | | STPFTF | |
| | | | NYE | | VPLVF | | | | | | | |
| | | | | | W | | | | | | | |
| 193 | FTFSNS | 1981 | SYISGT | 1984 | CAKDY | 1987 | RASQSI | 1990 | AASNL | 1993 | CQQYG | 1996 |
| | | | GSTIY | | DSSYG | | YNYLN | | QS | | NAPLT | |
| | DMN | | YA | | SGYYG | | | | | | F | |
| | | | | | MDVW | | | | | | | |

TABLE 16b
Sequences of Non-Selective Anti-CD33 Polypeptides
(VH and VL Sequences)

| Polypeptide | | SEQ ID | | SEQ ID |
|---|---|---|---|---|
| No. | Full VH | NO | Full VL | NO |
| 89 | QVQLVQSGAEVKKPGASVKV | 1035 | DIQMTQSPSSLSASVGDRVTI | 1090 |
| | SCKASGYTFTNYYMHWVRQA | | TCRASQGISNYLAWYQQKPG | |
| | PGQGLEWMGWINPNSGDTNY | | KAPKLLIYSASNLQSGVPSRF | |
| | EQKFQGRVTMTRDTSTSTVY | | SGSGSGTDFTLTISSLQPEDFA | |
| | MELSSLRSEDTAVYYCARDD | | TYYCQQSFSTPFTFGQGTKLE | |
| | RIQLWVPLVFWGQGTLVTVSS | | IKR | |
| 90 | EVQLLESGGGLVQPGGSLRLS | 1036 | DIQMTQSPSSLSASVGDRVTI | 1091 |
| | CAASGFTFSNSDMNWVRQAP | | TCRASQSIYNYLNWYQQKPG | |
| | GKGLEWVSYISGTGSTIYYAD | | KAPKLLIYAASNLQSGVPSRF | |
| | SVKGRFTISRDNSKNTLYLQM | | SGSGSGTDFTLTISSLQPEDFA | |
| | NSLRAEDTAVYYCAKDYDSS | | TYYCQQYGNAPLTFGQGTKV | |
| | YGSGYYGMDVWGQGTTVTV | | EIKR | |
| | SS | | | |
| 91 | QVQLVQSGAEVKKPGASVKV | 1037 | DIQMTQSPSSLSASVGDRVTI | 1092 |
| | SCKASGGTFSSYAISWVRQAP | | TCQASHDINIHLNWYQQKPG | |
| | GQGLEWMGWMNPNSGNTGY | | KAPKLLIYDASNLETGVPSRF | |
| | AQKFQGRVTMTRDTSTSTVY | | SGSGSGTDFTLTISSLQPEDFA | |
| | MELSSLRSEDTAVYYCAREDY | | TYYCQQAYSLPWTFGQGTKV | |
| | YDSSGNFDYWGQGTLVTVSS | | EIKR | |
| 92 | QVQLVQSGAEVKKPGASVKV | 1038 | DIVMTQSPLSLPVTPGEPASIS | 1093 |
| | SCKASGYTFTNHYMHWVRQA | | CRSSQSLLHSNGYNYLDWYL | |
| | PGQGLEWMGIINPSGGSTSYA | | QKPGQSPQLLIYAASSLQSGV | |
| | QKFQGRVTMTRDTSTSTVYM | | PDRFSGSGSGTDFTLKISRVE | |
| | ELSSLRSEDTAVYYCASAEVG | | AEDVGVYYCMQALHPPTFG | |
| | ATHYGMDVWGQGTTVTVSS | | QGTKVEIKR | |
| 93 | QVQLVQSGAEVKKPGASVKV | 1039 | DIVMTQSPLSLPVTPGEPASIS | 1094 |
| | SCKASGYSFTNYDISWVRQAP | | CRSSQSLLHSNGYNYLDWYL | |
| | GQGLEWMGWVNPNSGNTGY | | QKPGQSPQLLIYAASSLQSGV | |
| | AQKFQGRVTMTRDTSTSTVY | | PDRFSGSGSGTDFTLKISRVE | |
| | MELSSLRSEDTAVYYCAKDAP | | AEDVGVYYCMQALQTPLTFG | |
| | YYYDSSGYYGVFDYWGQGTL | | QGTRLEIKR | |
| | VTVSS | | | |
| 94 | QVQLVQSGAEVKKPGASVKV | 1040 | DIVMTQSPLSLPVTPGEPASIS | 1095 |
| | SCKASGYTFTTYDINWVRQAP | | CRSSQSLLHSNGYNYLDWYL | |
| | GQGLEWMGWISGYNGNTGY | | QKPGQSPQLLIYAASTLQSGV | |
| | AQKFQGRVTMTRDTSTSTVY | | PDRFSGSGSGTDFTLKISRVE | |
| | MELSSLRSEDTAVYYCAKDM | | AEDVGVYYCMQALQIPITFG | |
| | GYGDYPDAFDIWGQGTMVTV | | QGTKVEIKR | |
| | SS | | | |
| 95 | QVQLVQSGAEVKKPGSSVKV | 1041 | DIVMTQSPLSLPVTPGEPASIS | 1096 |
| | SCKASGYSFTTYDINWVRQAP | | CRSSQSLLHSNGYNYLDWYL | |
| | GQGLEWMGRMNPNSGNTGY | | QKPGQSPQLLIYLGSLRAPGV | |
| | AQKFQGRVTITADESTSTAYM | | PDRFSGSGSGTDFTLKISRVE | |
| | ELSSLRSEDTAVYYCARVVHG | | AEDVGVYYCMQALQTPWTF | |
| | MDVWGQGTTVTVSS | | GQGTKVEIKR | |
| 96 | QVQLVQSGAEVKKPGASVKV | 1042 | DIQMTQSPSSLSASVGDRVTI | 1097 |
| | SCKASGGTFSSYAISWVRQAP | | TCRASQSIGSWLAWYQQKPG | |
| | GQGLEWMGWMNPSSANTGY | | KAPKLLIYGVSTLHSGVPSRF | |
| | AQKFQGRVTMTRDTSTSTVY | | SGSGSGTDFTLTISSLQPEDFA | |
| | MELSSLRSEDTAVYYCAKDE | | TYYCQQSYSTPPTFGQGTKLE | |
| | MELLTAFDIWGQGTMVTVSS | | IKR | |
| 97 | EVQLLESGGGLVQPGGSLRLS | 1043 | DIQMTQSPSSLSASVGDRVTI | 1098 |
| | CAASGFTFRSYWMTWVRQAP | | TCRASQSISSYLNWYQQKPG | |
| | GKGLEWVSVISGSGDNTYYA | | KAPKLLIYDASNLETGVPSRF | |
| | DSVKGRFTISRDNSKNTLYLQ | | SGSGSGTDFTLTISSLQPEDFA | |
| | MNSLRAEDTAVYYCARETTW | | TYYCQQSSIIPLTFGGGTKVEI | |
| | GMDVWGQGTTVTVSS | | KR | |
| 98 | QVQLVQSGAEVKKPGASVKV | 1044 | DIQMTQSPSSLSASVGDRVTI | 1099 |
| | SCKASGGSFSSSAINWVRQAP | | TCRASQDIGSYLAWYQQKPG | |
| | GQGLEWMGWMNPNSGNTGY | | KAPKLLIYAASSLQSGVPSRF | |
| | AQKFQGRVTMTRDTSTSTVY | | SGSGSGTDFTLTISSLQPEDFA | |
| | MELSSLRSEDTAVYYCARDR | | TYYCHQSYSTPFTFGQGTKLE | |
| | GIAVAGASPYYYYGMDVWG | | IKR | |
| | QGTTVTVSS | | | |
| 99 | QVQLVQSGAEVKKPGSSVKV | 1045 | DIQMTQSPSSLSASVGDRVTI | 1100 |
| | SCKASGYTFSDYHIHWVRQAP | | TCRASQGISNNLNWYQQKPG | |
| | GQGLEWMGWMNPNSGNTGY | | KAPKLLIYQASNKDTGVPSRF | |
| | AQKFQGRVTITADESTSTAYM | | SGSGSGTDFTLTISSLQPEDFA | |
| | ELSSLRSEDTAVYYCARTHSS | | TYYCQQSYSSPPTFGQGTKVE | |
| | GYYYWFDPWGQGTLVTVSS | | IKR | |
| 100 | QVQLVQSGAEVKKPGASVKV | 1046 | DIQMTQSPSSLSASVGDRVTI | 1101 |
| | SCKASGATRSWMHWVRQAP | | TCRASQSISSWLAWYQQKPG | |
| | GQGLEWMGIINPSGDSTSYAQ | | KAPKLLIYGASSLQSGVPSRF | |
| | KFQGRVTMTRDTSTSTVYME | | SGSGSGTDFTLTISSLQPEDFA | |
| | LSSLRSEDTAVYYCAKEPYSS | | TYYCQQSYTTPITFGQGTRLE | |
| | SPYYFDYWGQGTLVTVSS | | IKR | |
| 101 | EVQLLESGGGLVQPGGSLRLS | 1047 | DIQMTQSPSSLSASVGDRVTI | 1102 |
| | CAASGFTFSSYGVHWVRQAP | | TCRASQNINTFLNWYQQKPG | |
| | GKGLEWVSAISGDGGDTYYA | | KAPKLLIYAASTLQSGVPSRF | |
| | DSVKGRFTISRDNSKNTLYLQ | | SGSGSGTDFTLTISSLQPEDFA | |
| | MNSLRAEDTAVYYCARDTW | | TYYCQQYDSFPLTFGGGTKV | |
| | DYSNYGGIDYWGQGTLVTVS | | EIKR | |
| | S | | | |
| 102 | EVQLLESGGGLVQPGGSLRLS | 1048 | DIVMTQSPLSLPVTPGEPASIS | 1103 |
| | CAASGFTFSNAWMSWVRQAP | | CRSSQSLLHSNGYNYLDWYL | |
| | GKGLEWVSGIGGSGGTIYYAD | | QKPGQSPQLLIYLGSNRASGV | |
| | SVKGRFTISRDNSKNTLYLQM | | PDRFSGSGSGTDFTLKISRVE | |
| | NSLRAEDTAVYYCAREVAAP | | AEDVGVYYCMQALETPITFG | |
| | LHPFGYYYYMDVWGKGTTV | | QGTRLEIKR | |
| | TVSS | | | |
| 103 | QVQLVQSGAEVKKPGASVKV | 1049 | DIVMTQSPLSLPVTPGEPASIS | 1104 |
| | SCKASGYTFTGYYMHWVRQA | | CRSSQSLLHSNGYNYLDWYL | |
| | PGQGLEWMGWMNPDSGDTN | | QKPGQSPQLLIYLGSNRASGV | |
| | YAQNFQGRVTMTRDTSTSTV | | PDRFSGSGSGTDFTLKISRVE | |
| | YMELSSLRSEDTAVYYCATTR | | AEDVGVYYCMQATHWPTFG | |
| | QPHYGMDVWGQGTTVTVSS | | QGTRLEIKR | |
| 104 | EVQLLESGGGLVQPGGSLRLS | 1050 | DIQMTQSPSSLSASVGDRVTI | 1105 |
| | CAASGFTFSSSWMHWVRQAP | | TCRASQGISSYLAWYQQKPG | |
| | GKGLEWVAVISYDGSEEYYA | | KAPKLLIYAASSLQSGVPSRF | |
| | DSVKGRFTISRDNSKNTLYLQ | | SGSGSGTDFTLTISSLQPEDFA | |
| | MNSLRAEDTAVYYCARLTDY | | TYYCQQSYSIPPTFGQGTKLEI | |
| | GDYVLGRYLSDWGQGTLVTV | | KR | |
| | SS | | | |
| 105 | EVQLLESGGGLVQPGGSLRLS | 1051 | DIQMTQSPSSLSASVGDRVTI | 1106 |
| | CAASGFTFNNAWMTWVRQA | | TCRASQSIYSWLAWYQQKPG | |
| | PGKGLEWVAVISYDGSNKYY | | KAPKLLIYAASTLQSGVPSRF | |
| | ADSVKGRFTISRDNSKNTLYL | | SGSGSGTDFTLTISSLQPEDFA | |
| | QMNSLRAEDTAVYYCARMA | | TYYCQQTYSTPVTFGQGTKV | |
| | VAGKGAFDIWGQGTMVTVSS | | EIKR | |
| 106 | QVQLVQSGAEVKKPGASVKV | 1052 | DIQMTQSPSSLSASVGDRVTI | 1107 |
| | SCKASGYTFTGYYMHWVRQA | | TCRASQSISWFLNWYQQKPG | |
| | PGQGLEWMGRIKPNSGGTDY | | KAPKLLIYAASSLQNGVPSRF | |
| | AQKFQGRVTMTRDTSTSTVY | | SGSGSGTDFTLTISSLQPEDFA | |
| | MELSSLRSEDTAVYYCARGA | | TYYCQQYGSFPPTFGGGTKV | |
| | YSGSYYGPIEYFQHWGQGTLV | | EIKR | |
| | TVSS | | | |
| 107 | QVQLVQSGAEVKKPGSSVKV | 1053 | DIQMTQSPSSLSASVGDRVTI | 1108 |
| | SCKASGYTFTDYYIHWVRQAP | | TCQASQDISNYLNWYQQKPG | |
| | GQGLEWMGGIIPIFGTANYAQ | | KAPKLLIYAASTLQSGVPSRF | |
| | KFQGRVTITADESTSTAYMEL | | SGSGSGTDFTLTISSLQPEDFA | |
| | SSLRSEDTAVYYCAREPLWFG | | TYYCQQSYSSPPTFGQGTKVE | |
| | ESSPHDYYGMDVWGQGTLVT | | IKR | |
| | VSS | | | |
| 108 | QVQLVQSGAEVKKPGASVKV | 1054 | DIVMTQSPDSLAVSLGERATI | 1109 |
| | SCKASGYTFTNYDINWVRQAP | | NCRSSQSLLYSSNNLNYLAW | |
| | GQGLEWMGWMNPNSGNTGL | | YQQKPGQPPKLLIYWASTRES | |
| | VEKFQGRVTMTRDTSTSTVY | | GVPDRFSGSGSGTDFTLTISSL | |
| | MELSSLRSEDTAVYYCARGW | | QAEDVAVYYCQQYYSNPLTF | |
| | GHGYGDYKFDYWGQGTLVT | | GQGTKVEIKR | |
| | VSS | | | |
| 109 | QVQLVQSGAEVKKPGSSVKV | 1055 | EIVMTQSPATLSVSPGERATL | 1110 |
| | SCKASGYTFTTYGISWVRQAP | | SCRASQSISSSSLAWYQQKPG | |
| | GQGLEWMGWMNPNSGNTGY | | QAPRLLIYGASTRATGIPARFS | |
| | AQKFQGRVTITADESTSTAYM | | GSGSGTEFTLTISSLQSEDFAV | |
| | ELSSLRSEDTAVYYCAREGGD | | YYCQQYETAPYTFGQGTKLE | |
| | GDYPDYWGQGTLVTVSS | | IKR | |
| 110 | QVQLVQSGAEVKKPGASVKV | 1056 | DIQMTQSPSSLSASVGDRVTI | 1111 |
| | SCKASGYTFTDYYVHWVRQA | | TCRASQRIGNWLAWYQQKP | |
| | PGQGLEWMGWMNPNSGNTG | | GKAPKLLIYAASSLQSGVPSR | |
| | YAQKFQGRVTMTRDTSTSTV | | FSGSGSGTDFTLTISSLQPEDF | |
| | YMELSSLRSEDTAVYYCARDF | | ATYYCQQSYSTPLTFGQGTK | |
| | IWVEGYLASPPPRFDYWGQGT | | LEIKR | |
| | LVTVSS | | | |
| 111 | QVQLVQSGAEVKKPGASVKV | 1057 | EIVMTQSPATLSVSPGERATL | 1112 |
| | SCKASGGTFTSYGISWVRQAP | | SCRASQSVAGSYLAWYQQKP | |
| | GQGLEWMGWINPNTGVTNY | | GQAPRLLIYGASTRATGIPAR | |
| | AQDFQGRVTMTRDTSTSTVY | | FSGSGSGTEFTLTISSLQSEDF | |
| | MELSSLRSEDTAVYYCANEQ | | AVYYCQQYYSTPLTFGGGTK | |
| | GGFDYWGQGTLVTVSS | | VEIKR | |
| 112 | QVQLVQSGAEVKKPGSSVKV | 1058 | DIQMTQSPSSLSASVGDRVTI | 1113 |
| | SCKASGSTLTGYDIHWVRQAP | | TCRSSESISSWLAWYQQKPG | |
| | GQGLEWMGGIIPFLGTASYAQ | | KAPKLLIYSASTLQSGVPSRFS | |
| | EFQGRVTITADESTSTAYMEL | | GSGSGTDFTLTISSLQPEDFAT | |
| | SSLRSEDTAVYYCARGGGSGY | | YYCQQSYSTPVTFGQGTRLEI | |
| | DLDYWGQGTLVTVSS | | KR | |
| 113 | QVQLVQSGAEVKKPGSSVKV | 1059 | DIQMTQSPSSLSASVGDRVTI | 1114 |
| | SCKASGGTFSSYDINWVRQAP | | TCRASQGIRNDIGWYQQKPG | |
| | GQGLEWMGGLIPVFGTTHYA | | KAPKLLIYAASTLQSGVPSRF | |
| | QNFQGRVTITADESTSTAYME | | SGSGSGTDFTLTISSLQPEDFA | |
| | LSSLRSEDTAVYYCATGLGVT | | TYYCQQTYMMPYTFGQGTK | |
| | TSNYYYGMDVWGQGTLVTV | | LEIKR | |
| | SS | | | |
| 114 | QVQLVQSGAEVKKPGASVKV | 1060 | DIQMTQSPSSLSASVGDRVTI | 1115 |
| | SCKASGGTFSKYAISWVRQAP | | TCRASQSIGNWLAWYQQKPG | |
| | GQGLEWMGWMNPNSGNTGY | | KAPKLLIYKASSLESGVPSRFS | |
| | AQKFQGRVTMTRDTSTSTVY | | GSGSGTDFTLTISSLQPEDFAT | |
| | MELSSLRSEDTAVYYCARDQ | | YYCQQSYNTPPTFGPGTKVDI | |
| | GLTGYFDLWGRGTLVTVSS | | KR | |
| 115 | QVQLVQSGAEVKKPGASVKV | 1061 | DIVMTQSPLSLPVTPGEPASIS | 1116 |
| | SCKASGYTFTGYYMHWVRQA | | CRSSQSLLHSNGYNYLDWYL | |
| | PGQGLEWMGIISPSGGSPTYA | | QKPGQSPQLLIYLGSNRASGV | |
| | QKFQGRVTMTRDTSTSTVYM | | PDRFSGSGSGTDFTLKISRVE | |
| | ELSSLRSEDTAVYYCAREGNG | | AEDVGVYYCMQALQTPYTF | |
| | GMDVWGQGTTVTVSS | | GQGTKLEIKR | |
| 116 | EVQLLESGGGLVQPGGSLRLS | 1062 | DIQMTQSPSSLSASVGDRVTI | 1117 |
| | CAASGFTFSNYAMAWVRQAP | | TCRASQSISSYLNWYQQKPG | |
| | GKGLEWVSAISGSGGGTYYA | | KAPKLLIYATSRLQSGVPSRF | |
| | DSVKGRFTISRDNSKNTLYLQ | | SGSGSGTDFTLTISSLQPEDFA | |
| | MNSLRAEDTAVYYCAREGGY | | TYYCQQGFNFPPTFGGGTKV | |
| | DPDYYYYGMDVWGQGTTVT | | EIKR | |
| | VSS | | | |
| 117 | EVQLLESGGGLVQPGGSLRLS | 1063 | DIQMTQSPSSLSASVGDRVTI | 1118 |
| | CAASGFTFGDYPMSWVRQAP | | TCRASQTIGTWLAWYQQKPG | |
| | GKGLEWVAGISYDGLNEHYA | | KAPKLLIYDASSLESGVPSRFS | |
| | DSVKGRFTISRDNSKNTLYLQ | | GSGSGTDFTLTISSLQPEDFAT | |
| | MNSLRAEDTAVYYCARDRDS | | YYCQQSYSTPPTFGQGTKVEI | |
| | GPSGFQHWGQGTLVTVSS | | KR | |
| 118 | EVQLVESGGGLVKPGGSLRLS | 1064 | DIVMTQSPLSLPVTPGEPASIS | 1119 |
| | CAASGFTISNAWMSWVRQAP | | CRSSQSLLHSNGYNYLDWYL | |
| | GKGLEWVAHIWNDGSQKYY | | QKPGQSPQLLIYAASSLQSGV | |
| | ADSVKGRFTISRDDSKNTLYL | | PDRFSGSGSGTDFTLKISRVE | |
| | QMNSLKTEDTAVYYCARDGA | | AEDVGVYYCMQGLQTPHTF | |
| | LGVGPDDYWGQGTLVTVSS | | GGGTKVEIKR | |
| 119 | QVQLVQSGAEVKKPGASVKV | 1065 | DIVMTQSPLSLPVTPGEPASIS | 1120 |
| | SCKASGNTLTNDHIHWVRQA | | CRSSQSLLHSNGYNYLDWYL | |
| | PGQGLEWMGWMNPNSGDTG | | QKPGQSPQLLIYMGSNRASG | |
| | YAQKFQGRVTMTRDTSTSTV | | VPDRFSGSGSGTDFTLKISRV | |
| | YMELSSLRSEDTAVYYCARA | | EAEDVGVYYCMQALETPTFG | |
| | GVDTAMVTYYYYGMDVWG | | QGTRLEIKR | |
| | QGTTVTVSS | | | |
| 120 | QVQLVQSGAEVKKPGASVKV | 1066 | DIQMTQSPSSLSASVGDRVTI | 1121 |
| | SCKASGYTFTTYYMHWVRQA | | TCRASQSIGTYLHWYQQKPG | |
| | PGQGLEWMGWMNPNSGNTG | | KAPKLLIYAISTLQNGVPSRFS | |
| | YAQKFQGRVTMTRDTSTSTV | | GSGSGTDFTLTISSLQPEDFAT | |
| | YMELSSLRSEDTAVYYCARG | | YYCQQSYSPPLTFGGGTKVEI | |
| | HKVDSGYDPYGMDVWGQGT | | KR | |
| | TVTVSS | | | |
| 121 | QVQLVQSGAEVKKPGASVKV | 1067 | DIQMTQSPSSLSASVGDRVTI | 1122 |
| | SCKASGDSFTDYYIHWVRQAP | | TCRASQNIGNWLAWYQQKP | |
| | GQGLEWMGWMNPNSGNTGY | | GKAPKLLIYAASTLQSGVPSR | |
| | AQQFQGRVTMTRDTSTSTVY | | FSGSGSGTDFTLTISSLQPEDF | |
| | MELSSLRSEDTAVYYCARDRE | | ATYYCQQSYNSITFGPGTKV | |
| | YSSSSRYFDLWGRGTLVTVSS | | DIKR | |
| 122 | QVQLVQSGAEVKKPGASVKV | 1068 | DIQMTQSPSSLSASVGDRVTI | 1123 |
| | SCKASGYTFTDYWLHWVRQA | | TCRASQSISSYLNWYQQKPG | |
| | PGQGLEWMGTINPSGGSTSYS | | KAPKLLIYAASSLQSGVPSRF | |
| | HKFQGRVTMTRDTSTSTVYM | | SGSGSGTDFTLTISSLQPEDFA | |
| | ELSSLRSEDTAVYYCAKEEEG | | TYYCQQSYSTPLTFGQGTKV | |
| | FWSGYAFDYWGQGTLVTVSS | | EIKR | |
| 123 | EVQLLESGGGLVQPGGSLRLS | 1069 | DIQMTQSPSSLSASVGDRVTI | 1124 |
| | CAASGFILGNAWMHWVRQAP | | TCQASQDINNYLNWYQQKPG | |
| | GKGLEWVASVSGDGSDENYA | | KAPKLLIYAASNLQSGVPSRF | |
| | DSVKGRFTISRDNSKNTLYLQ | | SGSGSGTDFTLTISSLQPEDFA | |
| | MNSLRAEDTAVYYCARDTHD | | TYYCQQTYSFPLTFGGGTKV | |
| | YGDYAPFDYWGQGTLVTVSS | | EIKR | |
| 124 | EVQLVESGGGLVKPGGSLRLS | 1070 | EIVMTQSPATLSVSPGERATL | 1125 |
| | CAASGFTFSSYWMHWVRQAP | | SCRASQSVSTYVAWYQQKPG | |
| | GKGLEWVAVIWYDGSNKYY | | QAPRLLIYGASTRATGIPARFS | |
| | ADSVKGRFTISRDDSKNTLYL | | GSGSGTEFTLTISSLQSEDFAV | |
| | QMNSLKTEDTAVYYCVRDGA | | YYCQQYYDTPLTFGGGTKVE | |
| | RSGMDVWGQGTTVTVSS | | IKR | |
| 125 | QVQLVQSGAEVKKPGASVKV | 1071 | DIQMTQSPSSLSASVGDRVTI | 1126 |
| | SCKASGYSFTTYDIHWVRQAP | | TCQASQDISNYLNWYQQKPG | |
| | GQGLEWMGWMNPNSGNTGY | | KAPKLLIYAASTLQSGVPSRF | |
| | AQKFQGRVTMTRDTSTSTVY | | SGSGSGTDFTLTISSLQPEDFA | |
| | MELSSLRSEDTAVYYCATDH | | TYYCQQYSYLPVTFGQGTKL | |
| | WVLGGFDYWGQGTLVTVSS | | EIKR | |
| 126 | EVQLLESGGGLVQPGGSLRLS | 1072 | DIQMTQSPSSLSASVGDRVTI | 1127 |
| | CAASGFTFTTYDMHWVRQAP | | TCRASQSISTWLAWYQQKPG | |
| | GKGLEWVAGINWNSVIIDYA | | KAPKLLIYAASSLQSGVPSRF | |
| | DSVKGRFTISRDNSKNTLYLQ | | SGSGSGTDFTLTISSLQPEDFA | |
| | MNSLRAEDTAVYYCAKDLLY | | TYYCQQSDTLPLTFGGGTKV | |
| | YYDSIGAFDIWGQGTMVTVSS | | EIKR | |
| 127 | QVQLVQSGAEVKKPGASVKV | 1073 | DIQMTQSPSSLSASVGDRVTI | 1128 |
| | SCKASGYTFTNHHMHWVRQA | | TCQASQDIRNFLNWYQQKPG | |
| | PGQGLEWMGMINPSGGSTSY | | KAPKLLIYGASTLHSGVPSRF | |
| | AQKFQGRVTMTRDTSTSTVY | | SGSGSGTDFTLTISSLQPEDFA | |
| | MELSSLRSEDTAVYYCARGRP | | TYYCQQSYSTPLTFGGGTKV | |
| | VDIVATYYFDYWGQGTLVTV | | EIKR | |
| | SS | | | |
| 128 | QVQLVQSGAEVKKPGASVKV | 1074 | DIQMTQSPSSLSASVGDRVTI | 1129 |
| | SCKASGYTFTNYYIHWVRQAP | | TCRASQGISSALAWYQQKPG | |
| | GQGLEWLGWTNPINGDTGSA | | KAPKLLIYAASSLQSGVPSRF | |
| | QKFQGRVTMTRDTSTSTVYM | | SGSGSGTDFTLTISSLQPEDFA | |
| | ELSSLRSEDTAVYYCAKEGQL | | TYYCQQSYSTPLTFGGGTKV | |
| | AWADYYYYMDVWGKGTTVT | | EIKR | |
| | VSS | | | |
| 129 | EVQLVESGGGLVKPGGSLRLS | 1075 | DIQMTQSPSSLSASVGDRVTI | 1130 |
| | CAASGFSLRNYWMHWVRQA | | TCRASQSISSYLNWYQQKPG | |
| | PGKGLEWVSGISGSGGSTYYA | | KAPKLLIYAASSLQSGVPSRF | |
| | DSVKGRFTISRDDSKNTLYLQ | | SGSGSGTDFTLTISSLQPEDFA | |
| | MNSLKTEDTAVYYCARDYTG | | TYYCQQSYSTPLTFGGGTKV | |
| | VVDYWGQGTLVTVSS | | EIKR | |
| 130 | QVQLVQSGAEVKKPGASVKV | 1076 | DIQMTQSPSSLSASVGDRVTI | 1131 |
| | SCKASGGTFSNYAISWVRQAP | | TCRASQSIGTWLAWYQQKPG | |
| | GQGLEWMAWMNPNSGNTGY | | KAPKLLIYGATRLLSGVPSRF | |
| | AQKFQGRVTMTRDTSTSTVY | | SGSGSGTDFTLTISSLQPEDFA | |
| | MELSSLRSEDTAVYYCARDGF | | TYYCQQSYSTPPTFGQGTKLE | |
| | IGFGELFSAFDIWGQGTMVTV | | IKR | |
| | SS | | | |
| 131 | QVQLVQSGAEVKKPGASVKV | 1077 | DIQMTQSPSSLSASVGDRVTI | 1132 |
| | SCKASGGTFSNYAINWVRQAP | | TCQASQDISDHLNWYQQKPG | |
| | GQGLEWMGWINPNSGGTDSA | | KAPKLLIYGASTLQSGVPSRF | |
| | QKFQGRVTMTRDTSTSTVYM | | SGSGSGTDFTLTISSLQPEDFA | |
| | ELSSLRSEDTAVYYCARDSSL | | TYYCQQAYSFPWTFGQGTKL | |
| | ALSYGGNSEYYYGMDVWGQ | | EIKR | |
| | GTTVTVSS | | | |
| 132 | EVQLLESGGGLVQPGGSLRLS | 1078 | EIVMTQSPATLSVSPGERATL | 1133 |
| | CAASGFTFNNYGMHWVRQAP | | SCRASQSVNSYLAWYQQKPG | |
| | GKGLEWVSAISGSGGSTYYAD | | QAPRLLIYGASTRATGIPARFS | |
| | SVKGRFTISRDNSKNTLYLQM | | GSGSGTEFTLTISSLQSEDFAV | |
| | NSLRAEDTAVYYCAREYMQQ | | YYCQQYGSSPLSFGGGTKVEI | |
| | PHGGMDVWGQGTTVTVSS | | KR | |
| 133 | EVQLLESGGGLVQPGGSLRLS | 1079 | DIQMTQSPSSLSASVGDRVTI | 1134 |
| | CAASGFTFSSSWMHWVRQAP | | TCRASQGISSYLAWYQQKPG | |
| | GKGLEWVSAISSSGDATYYAD | | KAPKLLIYTASSLQSGVPSRFS | |
| | SVKGRFTISRDNSKNTLYLQM | | GSGSGTDFTLTISSLQPEDFAT | |
| | NSLRAEDTAVYYCAKFSDGG | | YYCQQYDNLPITFGQGTRLEI | |
| | AGDSDYWGQGTLVTVSS | | KR | |
| 134 | QVQLVQSGAEVKKPGASVKV | 1080 | DIQMTQSPSSLSASVGDRVTI | 1135 |
| | SCKASGYTFDSYLLHWVRQA | | TCRASQSIDSWLAWYQQKPG | |
| | PGQGLEWMGMINPSGAGTTY | | KAPKLLIYAASSLQSGVPSRF | |
| | AQKFQGRVTMTRDTSTSTVY | | SGSGSGTDFTLTISSLQPEDFA | |
| | MELSSLRSEDTAVYYCAKEGS | | TYYCQQSYTTPITFGGGTKVE | |
| | IAAGYYFDSWGQGTLVTVSS | | IKR | |
| 135 | QVQLVQSGAEVKKPGASVKV | 1081 | DIVMTQSPDSLAVSLGERATI | 1136 |
| | SCKASGYSFTTYGITWVRQAP | | NCKSSQSVLYGSNNKNYLA | |
| | GQGLEWMGWINPNSGNAGY | | WYQQKPGQPPKLLIYWASTR | |
| | AQKFQGRVTMTRDTSTSTVY | | ESGVPDRFSGSGSGTDFTLTIS | |
| | MELSSLRSEDTAVYYCASDLA | | SLQAEDVAVYYCQQYYSTPL | |
| | GYSSGYFDLWGRGTLVTVSS | | TFGGGTKVEIKR | |
| 136 | QVQLVQSGAEVKKPGASVKV | 1082 | DIQMTQSPSSLSASVGDRVTI | 1137 |
| | SCKASGDTLTNHFVHWVRQA | | TCRASQRIGNWLAWYQQKP | |
| | PGQGLEWMGWMNPNSGNTG | | GKAPKLLIYAASSLQSGVPSR | |
| | YAQKFQGRVTMTRDTSTSTV | | FSGSGSGTDFTLTISSLQPEDF | |
| | YMELSSLRSEDTAVYYCARDP | | ATYYCQQSYSPPLTFGPGTKV | |
| | QMGAVAGGFDYWGQGTLVT | | DIKR | |
| | VSS | | | |
| 137 | QVQLVQSGAEVKKPGASVKV | 1083 | DIQMTQSPSSLSASVGDRVTI | 1138 |
| | SCKASGYTFTDYYIHWVRQAP | | TCRASQGISSYLAWYQQKPG | |
| | GQGLEWMGMVNPSGGSANY | | KAPKLLIYDASNLDTGVPSRF | |
| | AQKFQGRVTMTRDTSTSTVY | | SGSGSGTDFTLTISSLQPEDFA | |
| | MELSSLRSEDTAVYYCAKDSA | | TYYCQQSYSTPLTFGGGTKV | |
| | WQEPYYFDYWGQGTLVTVSS | | EIKR | |
| 138 | QVQLVQSGAEVKKPGASVKV | 1084 | EIVMTQSPATLSVSPGERATL | 1139 |
| | SCKASGYTFSSYDMHWVRQA | | SCRASQSVGSNLAWYQQKPG | |
| | PGQGLEWMGVINPGGGYTNY | | QAPRLLIYGASTRATGIPARFS | |
| | AQKFQGRVTMTRDTSTSTVY | | GSGSGTEFTLTISSLQSEDFAV | |
| | MELSSLRSEDTAVYYCARDEG | | YYCQQSHSLPPTFGQGTRLEI | |
| | WELLLDYWGQGTLVTVSS | | KR | |
| 139 | QVQLVQSGAEVKKPGASVKV | 1085 | DIQMTQSPSSLSASVGDRVTI | 1140 |
| | SCKASGYTLSDHDINWVRQAP | | TCRASQGIRNYLAWYQQKPG | |
| | GQGLEWMGWMNPSTGNTGY | | KAPKLLIYAASSLQSGVPSRF | |
| | AQKFQGRVTMTRDTSTSTVY | | SGSGSGTDFTLTISSLQPEDFA | |
| | MELSSLRSEDTAVYYCERDQL | | TYYCQQSYSIPLTFGGGTKVE | |
| | RFGAWFDPWGQGTLVTVSS | | IKR | |
| 140 | EVQLVESGGGLVKPGGSLRLS | 1086 | DIQMTQSPSSLSASVGDRVTI | 1141 |
| | CAASGITVSSSWMHWVRQAP | | TCRASQSISSWLAWYQQKPG | |
| | GKGLEWVSAIGTGGGTHYAD | | KAPKLLIYDASTLQSGVPSRF | |
| | SVKGRFTISRDDSKNTLYLQM | | SGSGSGTDFTLTISSLQPEDFA | |
| | NSLKTEDTAVYYCARDQGGQ | | TYYCQQSYSIPLTFGGGTKVE | |
| | IDHWGQGTLVTVSS | | IKR | |
| 141 | QVQLVQSGAEVKKPGASVKV | 1087 | DIQMTQSPSSLSASVGDRVTI | 1142 |
| | SCKASGGTFSSYAISWVRQAP | | TCRASQSISSYLNWYQQKPG | |
| | GQGLEWMGVISPNGDTTVYA | | KAPKLLIYAASSLHSGVPSRF | |
| | QKFQGRVTMTRDTSTSTVYM | | SGSGSGTDFTLTISSLQPEDFA | |
| | ELSSLRSEDTAVYYCARDRGV | | TYYCQQSYSPPITFGQGTRLEI | |
| | AHSYYYGMDVWGQGTLVTV | | KR | |
| | SS | | | |
| 142 | EVQLLESGGGLVQPGGSLRLS | 1088 | DIQMTQSPSSLSASVGDRVTI | 1143 |
| | CAASGFTFSSYWMHWVRQAP | | TCRASQSISSYLNWYQQKPG | |
| | GKGLEWVAVISYDGSDKYYA | | KAPKLLIYAASSLQSGVPSRF | |
| | DSVKGRFTISRDNSKNTLYLQ | | SGSGSGTDFTLTISSLQPEDFA | |
| | MNSLRAEDTAVYYCARGLGG | | TYYCQQSYSTPLTFGGGTKV | |
| | TTGTADFDYWGQGTLVTVSS | | EIKR | |
| 143 | QVQLVQSGAEVKKPGASVKV | 1089 | DIQMTQSPSSLSASVGDRVTI | 1144 |
| | SCKASGYTFTGYYMHWVRQA | | TCRASQSIGTYLSWYQQKPG | |
| | PGQGLEWMGWMNANSGNTG | | KAPKLLIYAASSLQSGVPSRF | |
| | FAQKFQGRVTMTRDTSTSTVY | | SGSGSGTDFTLTISSLQPEDFA | |
| | MELSSLRSEDTAVYYCARDSS | | TYYCQQSYSSPITFGQGTKLEI | |
| | SWLSGGGWFDPWGQGTLVTV | | KR | |
| | SS | | | |
| 191 | QVQLVQSGAEVKKPGASVKV | 1997 | DIQMTQSPSSLSASVGDRVTI | 2000 |
| | SCKASGGTFSSYAISWVRQAP | | TCQASHDINIHLNWYQQKPG | |
| | GQGLEWMGWMNPNSGNTGY | | KAPKLLIYDASNLETGVPSRF | |
| | AQKFQGRVTMTRDTSTSTVY | | SGSGSGTDFTLTISSLQPEDFA | |
| | MELSSLRSEDTAVYYCAREDY | | TYYCQQAYSLPWTFGQGTKV | |
| | YDSSGNFDYWGQGTLVTVSS | | EIKR | |
| 192 | QVQLVQSGAEVKKPGASVKV | 1998 | DIQMTQSPSSLSASVGDRVTI | 2001 |
| | SCKASGYTFTNYYMHWVRQA | | TCRASQGISNYLAWYQQKPG | |
| | PGQGLEWMGWINPNSGDTNY | | KAPKLLIYSASNLQSGVPSRF | |
| | EQKFQGRVTMTRDTSTSTVY | | SGSGSGTDFTLTISSLQPEDFA | |
| | MELSSLRSEDTAVYYCARDD | | TYYCQQSFSTPFTFGQGTKLE | |
| | RIQLWVPLVFWGQGTLVTVSS | | IKR | |
| 193 | EVQLLESGGGLVQPGGSLRLS | 1999 | DIQMTQSPSSLSASVGDRVTI | 2002 |
| | CAASGFTFSNSDMNWVRQAP | | TCRASQSIYNYLNWYQQKPG | |
| | GKGLEWVSYISGTGSTIYYAD | | KAPKLLIYAASNLQSGVPSRF | |
| | SVKGRFTISRDNSKNTLYLQM | | SGSGSGTDFTLTISSLQPEDFA | |
| | NSLRAEDTAVYYCAKDYDSS | | TYYCQQYGNAPLTFGQGTKV | |
| | YGSGYYGMDVWGQGTTVTV | | EIKR | |
| | SS | | | |

The polypeptides above were tested as disclosed above in Examples 4 and 5. Data is disclosed below in Table 16c, reporting FACS fold change over parental as (−), indicating <2 fold; (+), indicating 2-10 fold; (++), indicating 10-30 fold; and (+++), indicating >30 fold.

TABLE 16c
Polypeptide Activity (FACS and BLI)

| | CD33 Mutant | CD33 WT | | |
|---|---|---|---|---|
| | Geometric | Geometric | CD33 R69 | CD33 R69G |
| | Mean Fold | Mean Fold | BLI/Octet | BLI/Octet |
| Polypeptide | Change over | Change over | Binding Summary | Binding Summary |
| No. | Jurkat Parental | Jurkat Parental | (Yes/No/Ambiguous) | (Yes/No/Ambiguous) |
| 89 | ++ | ++ | Yes | Yes |
| 90 | +++ | +++ | Yes | Yes |
| 91 | ++ | +++ | Yes | Yes |
| 92 | + | + | Yes | Yes |
| 93 | + | + | Yes | Yes |
| 94 | ++ | + | Yes | Yes |
| 95 | + | + | Yes | Yes |
| 96 | +++ | +++ | Yes | Yes |
| 97 | + | ++ | Yes | Yes |
| 98 | +++ | ++ | Yes | Yes |
| 99 | ++ | +++ | Yes | Yes |
| 100 | ++ | ++ | Yes | Yes |
| 101 | +++ | +++ | Yes | Yes |
| 102 | ++ | ++ | Yes | Yes |
| 103 | ++ | ++ | Yes | Yes |
| 104 | +++ | +++ | Yes | Yes |
| 105 | ++ | ++ | Yes | Yes |
| 106 | + | + | Yes | Yes |
| 107 | ++ | ++ | Yes | Yes |
| 108 | ++ | ++ | Yes | Yes |
| 109 | ++ | ++ | Yes | Yes |
| 110 | +++ | +++ | Yes | Yes |
| 111 | ++ | +++ | Yes | Yes |
| 112 | + | + | Yes | Yes |
| 113 | ++ | +++ | Yes | Yes |
| 114 | ++ | +++ | Yes | Yes |
| 115 | ++ | ++ | Yes | Yes |
| 116 | +++ | +++ | Yes | Yes |
| 117 | + | + | Yes | Yes |
| 118 | + | ++ | Yes | Yes |
| 119 | +++ | ++ | Yes | Yes |
| 120 | +++ | +++ | Yes | Yes |
| 121 | ++ | ++ | Yes | Yes |
| 122 | +++ | ++ | Yes | Yes |
| 123 | +++ | +++ | Yes | Yes |
| 124 | +++ | +++ | Yes | Yes |
| 125 | +++ | +++ | Yes | Yes |
| 126 | ++ | +++ | Yes | Yes |
| 127 | ++ | ++ | Yes | Yes |
| 128 | ++ | ++ | Yes | Yes |
| 129 | ++ | ++ | Yes | Yes |
| 130 | ++ | ++ | Yes | Yes |
| 131 | ++ | +++ | Yes | Yes |
| 132 | ++ | + | Yes | Yes |
| 133 | + | + | Yes | Yes |
| 134 | +++ | +++ | Yes | Yes |
| 135 | +++ | ++ | Yes | Yes |
| 136 | ++ | ++ | Yes | Yes |
| 137 | +++ | +++ | Yes | Yes |
| 138 | ++ | ++ | Yes | Yes |
| 139 | +++ | +++ | Yes | Yes |
| 140 | + | + | Yes | Yes |
| 141 | +++ | ++ | Yes | Yes |
| 142 | + | + | Yes | Yes |
| 143 | + | + | Yes | Yes |
| 191 | ++ | +++ | Yes | Yes |
| 192 | ++ | ++ | Yes | Yes |
| 193 | +++ | +++ | Yes | Yes |

### Example 18: Identification of Non-Selective Anti-Human CLL-1 scFv Clones

The methods above in Example 1 have been used to discover non-selective anti-human CLL-1 scFv clones.

TABLE 17a
Sequences of Non-Selective Anti-CLL-1 Polypeptides (CDR Sequences)

| Poly- | | | | | | | | | | | | |
|---|---|---|---|---|---|---|---|---|---|---|---|---|
| peptide | | SEQ ID | | SEQ ID | | SEQ ID | | SEQ ID | | SEQ ID | | SEQ ID |
| No. | HCDR1 | NO | HCDR2 | NO | HCDR3 | NO | LCDR1 | NO | LCDR2 | NO | LCDR3 | NO |
| 144 | YTFTA | 1145 | GIIDPS | 1192 | CARGD | 1239 | RASQG | 1286 | DASSL | 1333 | CQQSY | 1380 |
| | YYMH | | GGSTS | | YGDYH | | ISSYLA | | QS | | STPITF | |
| | | | YA | | TLW | | | | | | | |
| 145 | GTFSTS | 1146 | GWIHP | 1193 | CARDL | 1240 | RVSQG | 1287 | DASNL | 1334 | CQQSY | 1381 |
| | YMH | | DDGNT | | GDYDT | | ISSYLN | | QA | | STPPTF | |
| | | | DYA | | FDIW | | | | | | | |
| 146 | YTFSG | 1147 | GWIDP | 1194 | CARDY | 1241 | RVSQG | 1288 | EASSL | 1335 | CQQSY | 1382 |
| | HYMH | | NSGGT | | PFYGD | | ISSYLN | | ES | | SIPFTF | |
| | | | NYA | | NDAFD | | | | | | | |
| | | | | | IW | | | | | | | |
| 147 | YTFTS | 1148 | GGIIPS | 1195 | CARGT | 1242 | RASQG | 1289 | DASNL | 1336 | CQQSY | 1383 |
| | YHIH | | GGSTS | | NDDHY | | ISNYLA | | ET | | STPLTF | |
| | | | YA | | DYW | | | | | | | |
| 148 | GTFTT | 1149 | GWMN | 1196 | CARGT | 1243 | RASQSI | 1290 | AASSL | 1337 | CQQSF | 1384 |
| | YGIS | | PFSDN | | GDDAF | | STWVA | | QS | | SIPLTF | |
| | | | TDYA | | DIW | | | | | | | |
| 149 | YTFTS | 1150 | GWMN | 1197 | CAREL | 1244 | RASQSI | 1291 | SASNL | 1338 | CQQAI | 1385 |
| | YDIN | | PNSGN | | EGEWF | | SSYLN | | QS | | SFPLTF | |
| | | | TGYA | | DPW | | | | | | | |
| 150 | YIFTSQ | 1151 | GWINP | 1198 | CARDP | 1245 | RASQG | 1292 | DASHL | 1339 | CQQNY | 1386 |
| | YIH | | NSGGT | | WGAY | | ISNNLN | | DT | | SPPPTF | |
| | | | NYA | | GGDAF | | | | | | | |
| | | | | | DIW | | | | | | | |
| 151 | YTFTD | 1152 | GWMN | 1199 | CARVD | 1246 | RASQG | 1293 | DASNL | 1340 | CQQSY | 1387 |
| | YYIH | | PNSGN | | TADY | | ISSWL | | QT | | STPLTF | |
| | | | TGYA | | MDVW | | A | | | | | |
| 152 | GTFST | 1153 | GWMN | 1200 | CAKED | 1247 | RASQSI | 1294 | DASNL | 1341 | CQQSH | 1388 |
| | NAIS | | PNSGN | | YGGNF | | GPWLA | | QA | | SLPLTF | |
| | | | TGYA | | DYW | | | | | | | |
| 153 | GAFSS | 1154 | GWMN | 1201 | CAAD | 1248 | RASQSI | 1295 | DASRL | 1342 | CQQSY | 1389 |
| | YALS | | PNSGN | | WMIGG | | SSWLA | | QS | | GIPLTF | |
| | | | TGYA | | DAFDI | | | | | | | |
| | | | | | W | | | | | | | |
| 154 | GTFSS | 1155 | GWINP | 1202 | CAGEV | 1249 | RASQG | 1296 | AASSL | 1343 | CQQSY | 1390 |
| | YGVT | | NTGGT | | GVGGY | | ISNWL | | QS | | SIPLTF | |
| | | | DYA | | DAFDI | | A | | | | | |
| | | | | | W | | | | | | | |
| 155 | YTFTS | 1156 | GWMN | 1203 | CARPE | 1250 | RASQSI | 1297 | DASNL | 1344 | CQQSF | 1391 |
| | YDIN | | PSSGD | | RSDAF | | GPWLA | | EA | | SSPLTF | |
| | | | SGYA | | DIW | | | | | | | |
| 156 | YTFTG | 1157 | GWMN | 1204 | CARGD | 1251 | RASQSI | 1298 | DAFTL | 1345 | CQQSY | 1392 |
| | YFIH | | PNSGN | | YADW | | STWLA | | ET | | STPLTF | |
| | | | TGYA | | FDPW | | | | | | | |
| 157 | YTFSD | 1158 | GIINPS | 1205 | CARG | 1252 | RASQG | 1299 | DASNL | 1346 | CQQTY | 1393 |
| | YYIH | | GGSTS | | MTDD | | ISSWL | | ET | | AIPLTF | |
| | | | YA | | AFDIW | | A | | | | | |
| 158 | DSFSS | 1159 | GWINP | 1206 | CARST | 1253 | RASQSI | 1300 | DASNL | 1347 | CQQSY | 1394 |
| | YGIS | | KSGAT | | AFDAF | | SSWLA | | ET | | STPLTF | |
| | | | TSA | | DIW | | | | | | | |
| 159 | YSFTA | 1160 | GIINPS | 1207 | CARGN | 1254 | RASQSI | 1301 | DASNL | 1348 | CQQSY | 1395 |
| | NYIH | | GGSTS | | YGDYV | | SSWLA | | ET | | GTPLTF | |
| | | | YA | | EDW | | | | | | | |
| 160 | GTFTS | 1161 | GWINP | 1208 | CARLV | 1255 | RASQSI | 1302 | AASSL | 1349 | CQQGY | 1396 |
| | YDIN | | HSGGT | | GGDAF | | SSWLA | | QG | | TTPLTF | |
| | | | NYA | | DIW | | | | | | | |
| 161 | YTFTS | 1162 | GMINP | 1209 | CAREL | 1256 | RASQG | 1303 | GASIL | 1350 | CQQSY | 1397 |
| | YDIN | | NSGGT | | LGESF | | ISSYLA | | QS | | STSFTF | |
| | | | SYA | | DYW | | | | | | | |
| 162 | YTFTN | 1163 | GWINP | 1210 | CARGT | 1257 | RASQSI | 1304 | AASTL | 1351 | CQQSY | 1398 |
| | YGIS | | NSGGT | | NGDEL | | SSYLA | | QS | | STPLTF | |
| | | | NFA | | DYW | | | | | | | |
| 163 | YTFTS | 1164 | GWMN | 1211 | CARAL | 1258 | RASQPI | 1305 | DTSSL | 1352 | CQQSY | 1399 |
| | YYMQ | | PNSGN | | YGDYL | | ATWLA | | QS | | SLPLGF | |
| | | | TGYA | | DIW | | | | | | | |
| 164 | YTFTA | 1165 | GIINPN | 1212 | CARDS | 1259 | QASQD | 1306 | ATSTL | 1353 | CQQSY | 1400 |
| | HYIH | | GGRTT | | DFWSG | | ISNFLN | | QS | | TTEWT | |
| | | | YA | | YYSDY | | | | | | F | |
| | | | | | YYGM | | | | | | | |
| | | | | | DVW | | | | | | | |
| 165 | YTFTS | 1166 | GWMN | 1213 | CARLS | 1260 | RASQFI | 1307 | DASSL | 1354 | CQQSY | 1401 |
| | YDIN | | PNSGN | | SGYYP | | ANWL | | ES | | STPLTF | |
| | | | TGYA | | DYW | | A | | | | | |
| 166 | YTFES | 1167 | GWIDP | 1214 | CARAD | 1261 | RASQG | 1308 | DASNL | 1355 | CQQSY | 1402 |
| | YDMN | | HSGDT | | YGGNA | | ISNWL | | ET | | STPYTF | |
| | | | NFA | | DYW | | A | | | | | |
| 167 | YTFTS | 1168 | GWINP | 1215 | CARGT | 1262 | RASQD | 1309 | AASSL | 1356 | CQQSY | 1403 |
| | YYMH | | NSGGT | | TGDDF | | ISTWL | | QS | | SIPPTF | |
| | | | NYA | | DYW | | A | | | | | |
| 168 | YTFTN | 1169 | GWINP | 1216 | CARVR | 1263 | RASQS | 1310 | AASTL | 1357 | CQQSY | 1404 |
| | YGIS | | NSGGT | | SDDFF | | VNHW | | QS | | SLPLTF | |
| | | | NYA | | DYW | | LA | | | | | |
| 169 | YTFTN | 1170 | GWMS | 1217 | CAKDN | 1264 | RVSQG | 1311 | DASNL | 1358 | CQQYD | 1405 |
| | DYIH | | PNSGK | | SSGWY | | ISSYLA | | ET | | TLPITF | |
| | | | TGFA | | FDLW | | | | | | | |
| 170 | GSFSN | 1171 | GWMN | 1218 | CARPR | 1265 | RASQSI | 1312 | EASTL | 1359 | CQQSY | 1406 |
| | HGVS | | PNSGD | | KDDAF | | SSWLA | | QS | | STPLTF | |
| | | | TGYA | | AIW | | | | | | | |
| 171 | YTFTD | 1172 | GMVDP | 1219 | CTSGS | 1266 | RASQSI | 1313 | EASNL | 1360 | CQQSY | 1407 |
| | YYIH | | NTGNI | | TNDAF | | GPWLA | | AS | | STPLTF | |
| | | | NYA | | DIW | | | | | | | |
| 172 | YTFSD | 1173 | GWMN | 1220 | CARGL | 1267 | RASQSI | 1314 | AASSL | 1361 | CQQSY | 1408 |
| | YYVH | | PNSGN | | TGDQF | | SSYLN | | QS | | STPLTF | |
| | | | TGYA | | DYW | | | | | | | |
| 173 | YTFNG | 1174 | GWINP | 1221 | CASLD | 1268 | RASQSI | 1315 | DASSL | 1362 | CQQSY | 1409 |
| | YNMH | | NSGDT | | YGDYA | | STWLA | | RS | | STPITF | |
| | | | NYA | | VYW | | | | | | | |
| 174 | FIFRDH | 1175 | SSIDFS | 1222 | CARDP | 1269 | RASQSI | 1316 | AASSL | 1363 | CQQTY | 1410 |
| | WMH | | TGYIY | | WGDG | | SSWLA | | QS | | TTPYTF | |
| | | | YA | | DFDY | | | | | | | |
| | | | | | W | | | | | | | |
| 175 | YTFTS | 1176 | GWINP | 1223 | CAGGP | 1270 | QASQD | 1317 | DASNL | 1364 | CQQAD | 1411 |
| | YDIH | | NSGNT | | DVDAA | | ISNYLN | | ET | | GFPPTF | |
| | | | GYA | | MVLD | | | | | | | |
| | | | | | YW | | | | | | | |
| 176 | GSFTS | 1177 | GWMN | 1224 | CARGA | 1271 | RASQN | 1318 | DGSNL | 1365 | CQQSY | 1412 |
| | YYIH | | PNSGN | | TDDAF | | IDTWL | | EA | | NTPITF | |
| | | | TGYA | | DIW | | A | | | | | |
| 177 | YTFTS | 1178 | GWMN | 1225 | CARST | 1272 | RASQSI | 1319 | DASNL | 1366 | CQQSY | 1413 |
| | YYMH | | PNSGN | | YSDSF | | SNWLA | | ET | | STPLTF | |
| | | | TGYA | | DYW | | | | | | | |
| 178 | FTFSSS | 1179 | SSITGS | 1226 | CIRDW | 1273 | RVSQG | 1320 | DASNL | 1367 | CQQGY | 1414 |
| | DMS | | GDGTY | | EGIYQ | | ISSYLN | | ET | | STPWT | |
| | | | YA | | W | | | | | | F | |
| 179 | GTFSS | 1180 | GTINPS | 1227 | CAIGG | 1274 | QASQD | 1321 | DASNL | 1368 | CQQGY | 1415 |
| | YAIS | | GGSTN | | YDSPY | | ISNYLN | | ET | | SPPWT | |
| | | | YA | | MDVW | | | | | | F | |
| 180 | YTFTSL | 1181 | GSMNP | 1228 | CAKSD | 1275 | RASQSI | 1322 | DASNL | 1369 | CQQSY | 1416 |
| | DIN | | RSGST | | YGDYL | | SPWLA | | QS | | STPLTF | |
| | | | AYA | | DYW | | | | | | | |
| 181 | YTFTG | 1182 | GVINPS | 1229 | CARGR | 1276 | RASQTI | 1323 | AASTL | 1370 | CQQSY | 1417 |
| | YYMH | | GGSTS | | TDDAF | | SSWLA | | QS | | SIPLTF | |
| | | | YA | | DIW | | | | | | | |
| 182 | YTFTD | 1183 | GIINTG | 1230 | CARGL | 1277 | RASQN | 1324 | EAFTL | 1371 | CQQSD | 1418 |
| | YYMH | | AGTTN | | TSDHF | | IGPWL | | QS | | NIPITF | |
| | | | YA | | DYW | | A | | | | | |
| 183 | GTFSS | 1184 | GGIIPK | 1231 | CARNS | 1278 | RASQSI | 1325 | AASSL | 1372 | CQQSY | 1419 |
| | YAIS | | FGPPN | | YGDDF | | SSWLA | | QR | | STPLTF | |
| | | | YA | | DYW | | | | | | | |
| 184 | GTFGN | 1185 | GVINPS | 1232 | CARSL | 1279 | RASQSI | 1326 | DASNL | 1373 | CQQSY | 1420 |
| | YGIN | | SGGTN | | GWPSP | | SRYLN | | ET | | STPWT | |
| | | | LA | | YMDV | | | | | | F | |
| | | | | | W | | | | | | | |
| 185 | FTFSNS | 1186 | SAISGS | 1233 | CARDD | 1280 | RASQD | 1327 | DASNL | 1374 | CQQSY | 1421 |
| | DMY | | DGTTY | | YGDQG | | IRNDL | | QT | | NMPYT | |
| | | | YA | | FDLW | | G | | | | F | |
| 186 | YTFTK | 1187 | GWINP | 1234 | CARDI | 1281 | QASQD | 1328 | DATNL | 1375 | CQQSY | 1422 |
| | YYMH | | NSGNT | | AVAGS | | ISNYLN | | ET | | STPPTF | |
| | | | GYA | | TYYYY | | | | | | | |
| | | | | | GMDV | | | | | | | |
| | | | | | W | | | | | | | |
| 187 | FTFSSY | 1188 | SSISSS | 1235 | CARDI | 1282 | QASQD | 1329 | GASSL | 1376 | CQQSY | 1423 |
| | DMH | | SSYIYY | | DDVAG | | ISNYLN | | QS | | STPFTF | |
| | | | A | | DYW | | | | | | | |
| 188 | FTFSSY | 1189 | SYTSSS | 1236 | CARGN | 1283 | QASQD | 1330 | DASNL | 1377 | CQQTY | 1424 |
| | GMH | | SSTIYY | | VGDN | | ISNYLN | | ET | | DTPYT | |
| | | | A | | WNDD | | | | | | F | |
| | | | | | EAFLG | | | | | | | |
| | | | | | W | | | | | | | |
| 189 | LTAGS | 1190 | SAISDD | 1237 | CVKDD | 1284 | RASQG | 1331 | GASSL | 1378 | CQQSY | 1425 |
| | NYMS | | GHWT | | GEGSG | | ISDYLA | | QS | | STPWT | |
| | | | DYA | | IDW | | | | | | F | |
| 190 | FTFSSS | 1191 | STINTN | 1238 | CARDT | 1285 | RASQS | 1332 | GASTR | 1379 | CQQYG | 1426 |
| | WMH | | GDAAY | | VLDDY | | VSSSY | | AA | | SSPFTF | |
| | | | YA | | GDYDD | | LA | | | | | |
| | | | | | YGMD | | | | | | | |
| | | | | | VW | | | | | | | |
| 194 | VTFSN | 2003 | GWMN | 2010 | CARGE | 2017 | RASQSI | 2024 | DASSL | 2031 | CQQSH | 2038 |
| | SGIN | | PASGD | | YGAEY | | SSWLA | | ES | | SLPPTF | |
| | | | TGYA | | FQHW | | | | | | | |
| 195 | YTFTN | 2004 | GIINPS | 2011 | CAKPT | 2018 | RASQG | 2025 | DASNL | 2032 | CQQSY | 2039 |
| | SYIH | | GDSTT | | TGDG | | ISNWL | | ET | | STPLTF | |
| | | | YA | | MDVW | | A | | | | | |
| 196 | YTLTN | 2005 | GWISP | 2012 | CTTDL | 2019 | RASRSI | 2026 | DASNL | 2033 | CQQSY | 2040 |
| | YYMH | | TDGKT | | LGDWF | | RSYLN | | ET | | NTPWT | |
| | | | KYA | | DPW | | | | | | F | |
| 197 | YTLTN | 2006 | GWMN | 2013 | CATAT | 2020 | RASQSI | 2027 | GASSL | 2034 | CQQSY | 2041 |
| | NWMH | | PNSGN | | ADDAF | | STWLA | | QS | | DIPITF | |
| | | | TGYA | | DIW | | | | | | | |
| 198 | FTFSTF | 2007 | ATISY | 2014 | CARLE | 2021 | RASQSI | 2028 | DASNL | 2035 | CQQAN | 2042 |
| | WMS | | DGSNQ | | LHEGR | | SSYLN | | ET | | SFPFTF | |
| | | | YYA | | FDYW | | | | | | | |
| 199 | DTFTG | 2008 | GWMN | 2015 | CTTDR | 2022 | RASQS | 2029 | DASSL | 2036 | CQQAN | 2043 |
| | YHIH | | PDSGS | | LYGDY | | VSSWL | | QS | | SFPFTF | |
| | | | TGYA | | FDYW | | A | | | | | |
| 200 | YTFTD | 2009 | PNSGN | 2016 | CAADD | 2023 | ISNYLN | 2030 | EASSL | 2037 | CQQTY | 2044 |
| | YYMH | | GWMN | | TKSPY | | QASQD | | QS | | SPPPTF | |
| | | | TGYA | | GMDV | | | | | | | |
| | | | | | W | | | | | | | |

TABLE 17b
Sequences of Non-Selective Anti-CLL-1 Polypeptides (VH and VL
Sequences)

| Polypeptide | VH | SEQ ID | | SEQ |
|---|---|---|---|---|
| No. | Full | NO | Full VL | ID NO |
| 144 | QVQLVQSGAEVKKPGASVKV | 1427 | DIQMTQSPSSLSASVGDRVTI | 1474 |
| | SCKASGYTFTAYYMHWVRQA | | TCRASQGISSYLAWYQQKPG | |
| | PGQGLEWMGIIDPSGGSTSYA | | KAPKLLIYDASSLQSGVPSRF | |
| | QQFQGRVTMTRDTSTSTVYM | | SGSGSGTDFTLTISSLQPEDFA | |
| | ELSSLRSEDTAVYYCARGDYG | | TYYCQQSYSTPITFGPGTKVD | |
| | DYHTLWGQGTLVTVSS | | IKR | |
| 145 | QVQLVQSGAEVKKPGASVKV | 1428 | DIQMTQSPSSLSASVGDRVTI | 1475 |
| | SCKASGGTFSTSYMHWVRQA | | TCRVSQGISSYLNWYQQKPG | |
| | PGQGLEWMGWIHPDDGNTDY | | KAPKLLIYDASNLQAGVPSRF | |
| | APKFQGRVTMTRDTSTSTVY | | SGSGSGTDFTLTISSLQPEDFA | |
| | MELSSLRSEDTAVYYCARDLG | | TYYCQQSYSTPPTFGQGTKV | |
| | DYDTFDIWGQGTMVTVSS | | EIKR | |
| 146 | QVQLVQSGAEVKKPGASVKV | 1429 | DIQMTQSPSSLSASVGDRVTI | 1476 |
| | SCKASGYTFSGHYMHWVRQA | | TCRVSQGISSYLNWYQQKPG | |
| | PGQGLEWMGWIDPNSGGTNY | | KAPKLLIYEASSLESGVPSRFS | |
| | AQKFQGRVTMTRDTSTSTVY | | GSGSGTDFTLTISSLQPEDFAT | |
| | MELSSLRSEDTAVYYCARDYP | | YYCQQSYSIPFTFGPGTKVDI | |
| | FYGDNDAFDIWGQGTTVTVSS | | KR | |
| 147 | QVQLVQSGAEVKKPGASVKV | 1430 | DIQMTQSPSSLSASVGDRVTI | 1477 |
| | SCKASGYTFTSYHIHWVRQAP | | TCRASQGISNYLAWYQQKPG | |
| | GQGLEWVGGIIPSGGSTSYAQ | | KAPKLLIYDASNLETGVPSRF | |
| | KFQGRVTMTRDTSTSTVYME | | SGSGSGTDFTLTISSLQPEDFA | |
| | LSSLRSEDTAVYYCARGTNDD | | TYYCQQSYSTPLTFGGGTKV | |
| | HYDYWGQGTLVTVSS | | EIKR | |
| 148 | QVQLVQSGAEVKKPGASVKV | 1431 | DIQMTQSPSSLSASVGDRVTI | 1478 |
| | SCKASGGTFTTYGISWVRQAP | | TCRASQSISTWVAWYQQKPG | |
| | GQGLEWMGWMNPFSDNTDY | | KAPKLLIYAASSLQSGVPSRF | |
| | AQNFQGRVTMTRDTSTSTVY | | SGSGSGTDFTLTISSLQPEDFA | |
| | MELSSLRSEDTAVYYCARGTG | | TYYCQQSFSIPLTFGQGTKVEI | |
| | DDAFDIWGQGTMVTVSS | | KR | |
| 149 | QVQLVQSGAEVKKPGASVKV | 1432 | DIQMTQSPSSLSASVGDRVTI | 1479 |
| | SCKASGYTFTSYDINWVRQAP | | TCRASQSISSYLNWYQQKPG | |
| | GQGLEWMGWMNPNSGNTGY | | KAPKLLIYSASNLQSGVPSRF | |
| | AQKFQGRVTMTRDTSTSTVY | | SGSGSGTDFTLTISSLQPEDFA | |
| | MELSSLRSEDTAVYYCARELE | | TYYCQQAISFPLTFGQGTKVE | |
| | GEWFDPWGQGTLVTVSS | | IKR | |
| 150 | QVQLVQSGAEVKKPGASVKV | 1433 | DIQMTQSPSSLSASVGDRVTI | 1480 |
| | SCKASGYIFTSQYIHWVRQAP | | TCRASQGISNNLNWYQQKPG | |
| | GQGLEWMGWINPNSGGTNYA | | KAPKLLIYDASHLDTGVPSRF | |
| | QKFQGRVTMTRDTSTSTVYM | | SGSGSGTDFTLTISSLQPEDFA | |
| | ELSSLRSEDTAVYYCARDPWG | | TYYCQQNYSPPPTFGQGTRLE | |
| | AYGGDAFDIWGQGTMVTVSS | | IKR | |
| 151 | QVQLVQSGAEVKKPGASVKV | 1434 | DIQMTQSPSSLSASVGDRVTI | 1481 |
| | SCKASGYTFTDYYIHWVRQAP | | TCRASQGISSWLAWYQQKPG | |
| | GQGLEWMGWMNPNSGNTGY | | KAPKLLIYDASNLQTGVPSRF | |
| | AQKFQGRVTMTRDTSTSTVY | | SGSGSGTDFTLTISSLQPEDFA | |
| | MELSSLRSEDTAVYYCARVDT | | TYYCQQSYSTPLTFGQGTKV | |
| | ADYMDVWGKGTLVTVSS | | EIKR | |
| 152 | QVQLVQSGAEVKKPGASVKV | 1435 | DIQMTQSPSSLSASVGDRVTI | 1482 |
| | SCKASGGTFSTNAISWVRQAP | | TCRASQSIGPWLAWYQQKPG | |
| | GQGLEWMGWMNPNSGNTGY | | KAPKLLIYDASNLQAGVPSRF | |
| | AQKFQGRVTMTRDTSTSTVY | | SGSGSGTDFTLTISSLQPEDFA | |
| | MELSSLRSEDTAVYYCAKED | | TYYCQQSHSLPLTFGPGTKV | |
| | YGGNFDYWGQGTLVTVSS | | DIKR | |
| 153 | QVQLVQSGAEVKKPGASVKV | 1436 | DIQMTQSPSSLSASVGDRVTI | 1483 |
| | SCKASGGAFSSYALSWVRQAP | | TCRASQSISSWLAWYQQKPG | |
| | GQGLEWMGWMNPNSGNTGY | | KAPKLLIYDASRLQSGVPSRF | |
| | AQKFQGRVTMTRDTSTSTVY | | SGSGSGTDFTLTISSLQPEDFA | |
| | MELSSLRSEDTAVYYCAADW | | TYYCQQSYGIPLTFGGGTKVE | |
| | MIGGDAFDIWGQGTTVTVSS | | IKR | |
| 154 | QVQLVQSGAEVKKPGASVKV | 1437 | DIQMTQSPSSLSASVGDRVTI | 1484 |
| | SCKASGGTFSSYGVTWVRQA | | TCRASQGISNWLAWYQQKPG | |
| | PGQGLEWMGWINPNTGGTDY | | KAPKLLIYAASSLQSGVPSRF | |
| | AQNFQGRVTMTRDTSTSTVY | | SGSGSGTDFTLTISSLQPEDFA | |
| | MELSSLRSEDTAVYYCAGEV | | TYYCQQSYSIPLTFGGGTKVE | |
| | GVGGYDAFDIWGQGTTVTVS | | IKR | |
| | S | | | |
| 155 | QVQLVQSGAEVKKPGASVKV | 1438 | DIQMTQSPSSLSASVGDRVTI | 1485 |
| | SCKASGYTFTSYDINWVRQAP | | TCRASQSIGPWLAWYQQKPG | |
| | GQGLEWMGWMNPSSGDSGY | | KAPKLLIYDASNLEAGVPSRF | |
| | AQKFQGRVTMTRDTSTSTVY | | SGSGSGTDFTLTISSLQPEDFA | |
| | MELSSLRSEDTAVYYCARPER | | TYYCQQSFSSPLTFGGGTKVE | |
| | SDAFDIWGQGTTVTVSS | | IKR | |
| 156 | QVQLVQSGAEVKKPGASVKV | 1439 | DIQMTQSPSSLSASVGDRVTI | 1486 |
| | SCKASGYTFTGYFIHWVRQAP | | TCRASQSISTWLAWYQQKPG | |
| | GQGLEWMGWMNPNSGNTGY | | KAPKLLIYDAFTLETGVPSRF | |
| | AQKFQGRVTMTRDTSTSTVY | | SGSGSGTDFTLTISSLQPEDFA | |
| | MELSSLRSEDTAVYYCARGD | | TYYCQQSYSTPLTFGQGTKV | |
| | YADWFDPWGQGTLVTVSS | | EIKR | |
| 157 | QVQLVQSGAEVKKPGASVKV | 1440 | DIQMTQSPSSLSASVGDRVTI | 1487 |
| | SCKASGYTFSDYYIHWVRQAP | | TCRASQGISSWLAWYQQKPG | |
| | GQGLEWMGIINPSGGSTSYAQ | | KAPKLLIYDASNLETGVPSRF | |
| | KFQGRVTMTRDTSTSTVYME | | SGSGSGTDFTLTISSLQPEDFA | |
| | LSSLRSEDTAVYYCARGMTD | | TYYCQQTYAIPLTFGGGTKLE | |
| | DAFDIWGQGTMVTVSS | | IKR | |
| 158 | QVQLVQSGAEVKKPGASVKV | 1441 | DIQMTQSPSSLSASVGDRVTI | 1488 |
| | SCKASGDSFSSYGISWVRQAP | | TCRASQSISSWLAWYQQKPG | |
| | GQGLEWMGWINPKSGATTSA | | KAPKLLIYDASNLETGVPSRF | |
| | QKFQGRVTMTRDTSTSTVYM | | SGSGSGTDFTLTISSLQPEDFA | |
| | ELSSLRSEDTAVYYCARSTAF | | TYYCQQSYSTPLTFGQGTKV | |
| | DAFDIWGQGTTVTVSS | | EIKR | |
| 159 | QVQLVQSGAEVKKPGASVKV | 1442 | DIQMTQSPSSLSASVGDRVTI | 1489 |
| | SCKASGYSFTANYIHWVRQAP | | TCRASQSISSWLAWYQQKPG | |
| | GQGLEWMGIINPSGGSTSYAQ | | KAPKLLIYDASNLETGVPSRF | |
| | KFQGRVTMTRDTSTSTVYME | | SGSGSGTDFTLTISSLQPEDFA | |
| | LSSLRSEDTAVYYCARGNYG | | TYYCQQSYGTPLTFGGGTKV | |
| | DYVEDWGQGTLVTVSS | | EIKR | |
| 160 | QVQLVQSGAEVKKPGASVKV | 1443 | DIQMTQSPSSLSASVGDRVTI | 1490 |
| | SCKASGGTFTSYDINWVRQAP | | TCRASQSISSWLAWYQQKPG | |
| | GQGLEWMGWINPHSGGTNYA | | KAPKLLIYAASSLQGGVPSRF | |
| | QKFQGRVTMTRDTSTSTVYM | | SGSGSGTDFTLTISSLQPEDFA | |
| | ELSSLRSEDTAVYYCARLVGG | | TYYCQQGYTTPLTFGPGTKV | |
| | DAFDIWGQGTMVTVSS | | DIKR | |
| 161 | QVQLVQSGAEVKKPGASVKV | 1444 | DIQMTQSPSSLSASVGDRVTI | 1491 |
| | SCKASGYTFTSYDINWVRQAP | | TCRASQGISSYLAWYQQKPG | |
| | GQGLEWMGMINPNSGGTSYA | | KAPKLLIYGASILQSGVPSRFS | |
| | QKFQGRVTMTRDTSTSTVYM | | GSGSGTDFTLTISSLQPEDFAT | |
| | ELSSLRSEDTAVYYCARELLG | | YYCQQSYSTSFTFGPGTKVDI | |
| | ESFDYWGQGTLVTVSS | | KR | |
| 162 | QVQLVQSGAEVKKPGASVKV | 1445 | DIQMTQSPSSLSASVGDRVTI | 1492 |
| | SCKASGYTFTNYGISWVRQAP | | TCRASQSISSYLAWYQQKPG | |
| | GQGLEWMGWINPNSGGTNFA | | KAPKLLIYAASTLQSGVPSRF | |
| | QKFQGRVTMTRDTSTSTVYM | | SGSGSGTDFTLTISSLQPEDFA | |
| | ELSSLRSEDTAVYYCARGTNG | | TYYCQQSYSTPLTFGQGTRLE | |
| | DELDYWGQGTLVTVSS | | IKR | |
| 163 | QVQLVQSGAEVKKPGASVKV | 1446 | DIQMTQSPSSLSASVGDRVTI | 1493 |
| | SCKASGYTFTSYYMQWVRQA | | TCRASQPIATWLAWYQQKPG | |
| | PGQGLEWMGWMNPNSGNTG | | KAPKLLIYDTSSLQSGVPSRFS | |
| | YAQKFQGRVTMTRDTSTSTV | | GSGSGTDFTLTISSLQPEDFAT | |
| | YMELSSLRSEDTAVYYCARAL | | YYCQQSYSLPLGFGQGTKVEI | |
| | YGDYLDIWGQGTTVTVSS | | KR | |
| 164 | QVQLVQSGAEVKKPGASVKV | 1447 | DIQMTQSPSSLSASVGDRVTI | 1494 |
| | SCKASGYTFTAHYIHWVRQAP | | TCQASQDISNFLNWYQQKPG | |
| | GQGLEWMGIINPNGGRTTYA | | KAPKLLIYATSTLQSGVPSRF | |
| | QKFQGRVTMTRDTSTSTVYM | | SGSGSGTDFTLTISSLQPEDFA | |
| | ELSSLRSEDTAVYYCARDSDF | | TYYCQQSYTTEWTFGQGTKV | |
| | WSGYYSDYYYGMDVWGQGT | | EIKR | |
| | TVTVSS | | | |
| 165 | QVQLVQSGAEVKKPGASVKV | 1448 | DIQMTQSPSSLSASVGDRVTI | 1495 |
| | SCKASGYTFTSYDINWVRQAP | | TCRASQFIANWLAWYQQKPG | |
| | GQGLEWMGWMNPNSGNTGY | | KAPKLLIYDASSLESGVPSRFS | |
| | AQKFQGRVTMTRDTSTSTVY | | GSGSGTDFTLTISSLQPEDFAT | |
| | MELSSLRSEDTAVYYCARLSS | | YYCQQSYSTPLTFGGGTKVEI | |
| | GYYPDYWGQGTLVTVSS | | KR | |
| 166 | QVQLVQSGAEVKKPGASVKV | 1449 | DIQMTQSPSSLSASVGDRVTI | 1496 |
| | SCKASGYTFESYDMNWVRQA | | TCRASQGISNWLAWYQQKPG | |
| | PGQGLEWMGWIDPHSGDTNF | | KAPKLLIYDASNLETGVPSRF | |
| | AQKFQGRVTMTRDTSTSTVY | | SGSGSGTDFTLTISSLQPEDFA | |
| | MELSSLRSEDTAVYYCARAD | | TYYCQQSYSTPYTFGQGTKV | |
| | YGGNADYWGQGTLVTVSS | | EIKR | |
| 167 | QVQLVQSGAEVKKPGASVKV | 1450 | DIQMTQSPSSLSASVGDRVTI | 1497 |
| | SCKASGYTFTSYYMHWVRQA | | TCRASQDISTWLAWYQQKPG | |
| | PGQGLEWMGWINPNSGGTNY | | KAPKLLIYAASSLQSGVPSRF | |
| | AQKFQGRVTMTRDTSTSTVY | | SGSGSGTDFTLTISSLQPEDFA | |
| | MELSSLRSEDTAVYYCARGTT | | TYYCQQSYSIPPTFGPGTKVD | |
| | GDDFDYWGQGTLVTVSS | | IKR | |
| 168 | QVQLVQSGAEVKKPGASVKV | 1451 | DIQMTQSPSSLSASVGDRVTI | 1498 |
| | SCKASGYTFTNYGISWVRQAP | | TCRASQSVNHWLAWYQQKP | |
| | GQGLEWMGWINPNSGGTNYA | | GKAPKLLIYAASTLQSGVPSR | |
| | QKFQGRVTMTRDTSTSTVYM | | FSGSGSGTDFTLTISSLQPEDF | |
| | ELSSLRSEDTAVYYCARVRSD | | ATYYCQQSYSLPLTFGGGTK | |
| | DFFDYWGQGTLVTVSS | | VEIKR | |
| 169 | QVQLVQSGAEVKKPGASVKV | 1452 | DIQMTQSPSSLSASVGDRVTI | 1499 |
| | SCKASGYTFTNDYIHWVRQAP | | TCRVSQGISSYLAWYQQKPG | |
| | GQGLEWMGWMSPNSGKTGF | | KAPKLLIYDASNLETGVPSRF | |
| | AQKFQGRVTMTRDTSTSTVY | | SGSGSGTDFTLTISSLQPEDFA | |
| | MELSSLRSEDTAVYYCAKDNS | | TYYCQQYDTLPITFGQGTRLE | |
| | SGWYFDLWGRGTLVTVSS | | IKR | |
| 170 | QVQLVQSGAEVKKPGASVKV | 1453 | DIQMTQSPSSLSASVGDRVTI | 1500 |
| | SCKASGGSFSNHGVSWVRQA | | TCRASQSISSWLAWYQQKPG | |
| | PGQGLEWMGWMNPNSGDTG | | KAPKLLIYEASTLQSGVPSRF | |
| | YAQKFQGRVTMTRDTSTSTV | | SGSGSGTDFTLTISSLQPEDFA | |
| | YMELSSLRSEDTAVYYCARPR | | TYYCQQSYSTPLTFGQGTKV | |
| | KDDAFAIWGQGTLVTVSS | | EIKR | |
| 171 | QVQLVQSGAEVKKPGASVKV | 1454 | DIQMTQSPSSLSASVGDRVTI | 1501 |
| | SCKASGYTFTDYYIHWVRQAP | | TCRASQSIGPWLAWYQQKPG | |
| | GQGLEWMGMVDPNTGNINY | | KAPKLLIYEASNLASGVPSRF | |
| | AQTFQGRVTMTRDTSTSTVY | | SGSGSGTDFTLTISSLQPEDFA | |
| | MELSSLRSEDTAVYYCTSGST | | TYYCQQSYSTPLTFGGGTKV | |
| | NDAFDIWGQGTMVTVSS | | EIKR | |
| 172 | QVQLVQSGAEVKKPGASVKV | 1455 | DIQMTQSPSSLSASVGDRVTI | 1502 |
| | SCKASGYTFSDYYVHWVRQA | | TCRASQSISSYLNWYQQKPG | |
| | PGQGLEWMGWMNPNSGNTG | | KAPKLLIYAASSLQSGVPSRF | |
| | YAQKFQGRVTMTRDTSTSTV | | SGSGSGTDFTLTISSLQPEDFA | |
| | YMELSSLRSEDTAVYYCARGL | | TYYCQQSYSTPLTFGGGTKV | |
| | TGDQFDYWGQGTLVTVSS | | EIKR | |
| 173 | QVQLVQSGAEVKKPGASVKV | 1456 | DIQMTQSPSSLSASVGDRVTI | 1503 |
| | SCKASGYTFNGYNMHWVRQ | | TCRASQSISTWLAWYQQKPG | |
| | APGQGLEWMGWINPNSGDTN | | KAPKLLIYDASSLRSGVPSRF | |
| | YAQKFQGRVTMTRDTSTSTV | | SGSGSGTDFTLTISSLQPEDFA | |
| | YMELSSLRSEDTAVYYCASLD | | TYYCQQSYSTPITFGQGTKVE | |
| | YGDYAVYWGQGTLVTVSS | | IKR | |
| 174 | EVQLLESGGGLVQPGGSLRLS | 1457 | DIQMTQSPSSLSASVGDRVTI | 1504 |
| | CAASGFIFRDHWMHWVRQAP | | TCRASQSISSWLAWYQQKPG | |
| | GKGLEWVSSIDFSTGYIYYAD | | KAPKLLIYAASSLQSGVPSRF | |
| | SVKGRFTISRDNSKNTLYLQM | | SGSGSGTDFTLTISSLQPEDFA | |
| | NSLRAEDTAVYYCARDPWGD | | TYYCQQTYTTPYTFGQGTRL | |
| | GDFDYWGRGTLVTVSS | | EIKR | |
| 175 | QVQLVQSGAEVKKPGASVKV | 1458 | DIQMTQSPSSLSASVGDRVTI | 1505 |
| | SCKASGYTFTSYDIHWVRQAP | | TCQASQDISNYLNWYQQKPG | |
| | GQGLEWMGWINPNSGNTGYA | | KAPKLLIYDASNLETGVPSRF | |
| | QKFQGRVTMTRDTSTSTVYM | | SGSGSGTDFTLTISSLQPEDFA | |
| | ELSSLRSEDTAVYYCAGGPDV | | TYYCQQADGFPPTFGGGTKV | |
| | DAAMVLDYWGQGTLVTVSS | | EIKR | |
| 176 | QVQLVQSGAEVKKPGASVKV | 1459 | DIQMTQSPSSLSASVGDRVTI | 1506 |
| | SCKASGGSFTSYYIHWVRQAP | | TCRASQNIDTWLAWYQQKP | |
| | GQGLEWVGWMNPNSGNTGY | | GKAPKLLIYDGSNLEAGVPSR | |
| | AQKFQGRVTMTRDTSTSTVY | | FSGSGSGTDFTLTISSLQPEDF | |
| | MELSSLRSEDTAVYYCARGAT | | ATYYCQQSYNTPITFGQGTRL | |
| | DDAFDIWGQGTMVTVSS | | EIKR | |
| 177 | QVQLVQSGAEVKKPGASVKV | 1460 | DIQMTQSPSSLSASVGDRVTI | 1507 |
| | SCKASGYTFTSYYMHWVRQA | | TCRASQSISNWLAWYQQKPG | |
| | PGQGLEWMGWMNPNSGNTG | | KAPKLLIYDASNLETGVPSRF | |
| | YAQKFQGRVTMTRDTSTSTV | | SGSGSGTDFTLTISSLQPEDFA | |
| | YMELSSLRSEDTAVYYCARST | | TYYCQQSYSTPLTFGQGTKLE | |
| | YSDSFDYWGQGTLVTVSS | | IKR | |
| 178 | EVQLLESGGGLVQPGGSLRLS | 1461 | DIQMTQSPSSLSASVGDRVTI | 1508 |
| | CAASGFTFSSSDMSWVRQAPG | | TCRVSQGISSYLNWYQQKPG | |
| | KGLEWVSSITGSGDGTYYADS | | KAPKLLIYDASNLETGVPSRF | |
| | VKGRFTISRDNSKNTLYLQMN | | SGSGSGTDFTLTISSLQPEDFA | |
| | SLRAEDTAVYYCIRDWEGIYQ | | TYYCQQGYSTPWTFGQGTKL | |
| | WGQGTLVTVSS | | EIKR | |
| 179 | QVQLVQSGAEVKKPGASVKV | 1462 | DIQMTQSPSSLSASVGDRVTI | 1509 |
| | SCKASGGTFSSYAISWVRQAP | | TCQASQDISNYLNWYQQKPG | |
| | GQGLEWMGTINPSGGSTNYA | | KAPKLLIYDASNLETGVPSRF | |
| | QKFQGRVTMTRDTSTSTVYM | | SGSGSGTDFTLTISSLQPEDFA | |
| | ELSSLRSEDTAVYYCAIGGYD | | TYYCQQGYSPPWTFGGGTKV | |
| | SPYMDVWGKGTTVTVSS | | EIKR | |
| 180 | QVQLVQSGAEVKKPGASVKV | 1463 | DIQMTQSPSSLSASVGDRVTI | 1510 |
| | SCKASGYTFTSLDINWVRQAP | | TCRASQSISPWLAWYQQKPG | |
| | GQGLEWMGSMNPRSGSTAYA | | KAPKLLIYDASNLQSGVPSRF | |
| | QSFQGRVTMTRDTSTSTVYM | | SGSGSGTDFTLTISSLQPEDFA | |
| | ELSSLRSEDTAVYYCAKSDYG | | TYYCQQSYSTPLTFGQGTKLE | |
| | DYLDYWGQGTLVTVSS | | IKR | |
| 181 | QVQLVQSGAEVKKPGASVKV | 1464 | DIQMTQSPSSLSASVGDRVTI | 1511 |
| | SCKASGYTFTGYYMHWVRQA | | TCRASQTISSWLAWYQQKPG | |
| | PGQGLEWMGVINPSGGSTSYA | | KAPKLLIYAASTLQSGVPSRF | |
| | QKFQGRVTMTRDTSTSTVYM | | SGSGSGTDFTLTISSLQPEDFA | |
| | ELSSLRSEDTAVYYCARGRTD | | TYYCQQSYSIPLTFGGGTKLE | |
| | DAFDIWGQGTLVTVSS | | IKR | |
| 182 | QVQLVQSGAEVKKPGASVKV | 1465 | DIQMTQSPSSLSASVGDRVTI | 1512 |
| | SCKASGYTFTDYYMHWVRQA | | TCRASQNIGPWLAWYQQKPG | |
| | PGQGLEWLGIINTGAGTTNYA | | KAPKLLIYEAFTLQSGVPSRF | |
| | PKFQGRVTMTRDTSTSTVYM | | SGSGSGTDFTLTISSLQPEDFA | |
| | ELSSLRSEDTAVYYCARGLTS | | TYYCQQSDNIPITFGQGTKVE | |
| | DHFDYWGQGTLVTVSS | | IKR | |
| 183 | QVQLVQSGAEVKKPGSSVKV | 1466 | DIQMTQSPSSLSASVGDRVTI | 1513 |
| | SCKASGGTFSSYAISWVRQAP | | TCRASQSISSWLAWYQQKPG | |
| | GQGLEWMGGIIPKFGPPNYAP | | KAPKLLIYAASSLQRGVPSRF | |
| | KFQGRVTITADESTSTAYMEL | | SGSGSGTDFTLTISSLQPEDFA | |
| | SSLRSEDTAVYYCARNSYGDD | | TYYCQQSYSTPLTFGQGTKV | |
| | FDYWGQGTLVTVSS | | EIKR | |
| 184 | QVQLVQSGAEVKKPGASVKV | 1467 | DIQMTQSPSSLSASVGDRVTI | 1514 |
| | SCKASGGTFGNYGINWVRQA | | TCRASQSISRYLNWYQQKPG | |
| | PGQGLEWMGVINPSSGGTNL | | KAPKLLIYDASNLETGVPSRF | |
| | AQKFQGRVTMTRDTSTSTVY | | SGSGSGTDFTLTISSLQPEDFA | |
| | MELSSLRSEDTAVYYCARSLG | | TYYCQQSYSTPWTFGQGTKL | |
| | WPSPYMDVWGQGTMVTVSS | | EIKR | |
| 185 | EVQLLESGGGLVQPGGSLRLS | 1468 | DIQMTQSPSSLSASVGDRVTI | 1515 |
| | CAASGFTFSNSDMYWVRQAP | | TCRASQDIRNDLGWYQQKPG | |
| | GKGLEWVSAISGSDGTTYYA | | KAPKLLIYDASNLQTGVPSRF | |
| | DSVKGRFTISRDNSKNTLYLQ | | SGSGSGTDFTLTISSLQPEDFA | |
| | MNSLRAEDTAVYYCARDDYG | | TYYCQQSYNMPYTFGQGTKL | |
| | DQGFDLWGRGTLVTVSS | | EIKR | |
| 186 | QVQLVQSGAEVKKPGASVKV | 1469 | DIQMTQSPSSLSASVGDRVTI | 1516 |
| | SCKASGYTFTKYYMHWVRQA | | TCQASQDISNYLNWYQQKPG | |
| | PGQGLEWMGWINPNSGNTGY | | KAPKLLIYDATNLETGVPSRF | |
| | AQKFQGRVTMTRDTSTSTVY | | SGSGSGTDFTLTISSLQPEDFA | |
| | MELSSLRSEDTAVYYCARDIA | | TYYCQQSYSTPPTFGGGTKV | |
| | VAGSTYYYYGMDVWGQGTT | | EIKR | |
| | VTVSS | | | |
| 187 | EVQLLESGGGLVQPGGSLRLS | 1470 | DIQMTQSPSSLSASVGDRVTI | 1517 |
| | CAASGFTFSSYDMHWVRQAP | | TCQASQDISNYLNWYQQKPG | |
| | GKGLEWVSSISSSSSYIYYADS | | KAPKLLIYGASSLQSGVPSRF | |
| | VKGRFTISRDNSKNTLYLQMN | | SGSGSGTDFTLTISSLQPEDFA | |
| | SLRAEDTAVYYCARDIDDVA | | TYYCQQSYSTPFTFGQGTKLE | |
| | GDYWGQGTLVTVSS | | IKR | |
| 188 | EVQLLESGGGLVQPGGSLRLS | 1471 | DIQMTQSPSSLSASVGDRVTI | 1518 |
| | CAASGFTFSSYGMHWVRQAP | | TCQASQDISNYLNWYQQKPG | |
| | GKGLEWVSYTSSSSSTIYYAD | | KAPKLLIYDASNLETGVPSRF | |
| | SVKGRFTISRDNSKNTLYLQM | | SGSGSGTDFTLTISSLQPEDFA | |
| | NSLRAEDTAVYYCARGNVGD | | TYYCQQTYDTPYTFGQGTKL | |
| | NWNDDEAFLGWGQGTLVTVS | | EIKR | |
| | S | | | |
| 189 | EVQLLESGGGLVQPGGSLRLS | 1472 | DIQMTQSPSSLSASVGDRVTI | 1519 |
| | CAASGLTAGSNYMSWVRQAP | | TCRASQGISDYLAWYQQKPG | |
| | GKGLEWVSAISDDGHWTDYA | | KAPKLLIYGASSLQSGVPSRF | |
| | DSVKGRFTISRDNSKNTLYLQ | | SGSGSGTDFTLTISSLQPEDFA | |
| | MNSLRAEDTAVYYCVKDDGE | | TYYCQQSYSTPWTFGPGTKV | |
| | GSGIDWGQGTLVTVSS | | DIKR | |
| 190 | EVQLLESGGGLVQPGGSLRLS | 1473 | EIVMTQSPATLSVSPGERATL | 1520 |
| | CAASGFTFSSSWMHWVRQAP | | SCRASQSVSSSYLAWYQQKP | |
| | GKGLEWVSTINTNGDAAYYA | | GQAPRLLIYGASTRAAGIPAR | |
| | DSVKGRFTISRDNSKNTLYLQ | | FSGSGSGTEFTLTISSLQSEDF | |
| | MNSLRAEDTAVYYCARDTVL | | AVYYCQQYGSSPFTFGPGTK | |
| | DDYGDYDDYGMDVWGQGTT | | VDIKR | |
| | VTVSS | | | |
| 194 | QVQLVQSGAEVKKPGASVKV | 2045 | DIQMTQSPSSLSASVGDRVTI | 2052 |
| | SCKASGVTFSNSGINWVRQAP | | TCRASQSISSWLAWYQQKPG | |
| | GQGLEWMGWMNPASGDTGY | | KAPKLLIYDASSLESGVPSRFS | |
| | AQKFQGRVTMTRDTSTSTVY | | GSGSGTDFTLTISSLQPEDFAT | |
| | MELSSLRSEDTAVYYCARGEY | | YYCQQSHSLPPTFGQGTRLEI | |
| | GAEYFQHWGQGTLVTVSS | | KR | |
| 195 | QVQLVQSGAEVKKPGASVKV | 2046 | DIQMTQSPSSLSASVGDRVTI | 2053 |
| | SCKASGYTFTNSYIHWVRQAP | | TCRASQGISNWLAWYQQKPG | |
| | GQGLEWMGIINPSGDSTTYAQ | | KAPKLLIYDASNLETGVPSRF | |
| | KFQGRVTMTRDTSTSTVYME | | SGSGSGTDFTLTISSLQPEDFA | |
| | LSSLRSEDTAVYYCAKPTTGD | | TYYCQQSYSTPLTFGGGTKV | |
| | GMDVWGQGTTVTVSS | | EIKR | |
| 196 | QVQLVQSGAEVKKPGASVKV | 2047 | DIQMTQSPSSLSASVGDRVTI | 2054 |
| | SCKASGYTLTNYYMHWVRQ | | TCRASRSIRSYLNWYQQKPG | |
| | APGQGLEWMGWISPTDGKTK | | KAPKLLIYDASNLETGVPSRF | |
| | YAQKFQGRVTMTRDTSTSTV | | SGSGSGTDFTLTISSLQPEDFA | |
| | YMELSSLRSEDTAVYYCTTDL | | TYYCQQSYNTPWTFGQGTKV | |
| | LGDWFDPWGQGTLVTVSS | | EIKR | |
| 197 | QVQLVQSGAEVKKPGASVKV | 2048 | DIQMTQSPSSLSASVGDRVTI | 2055 |
| | SCKASGYTLTNNWMHWVRQ | | TCRASQSISTWLAWYQQKPG | |
| | APGQGLEWMGWMNPNSGNT | | KAPKLLIYGASSLQSGVPSRF | |
| | GYAQKFQGRVTMTRDTSTST | | SGSGSGTDFTLTISSLQPEDFA | |
| | VYMELSSLRSEDTAVYYCAT | | TYYCQQSYDIPITFGPGTKVDI | |
| | ATADDAFDIWGQGTMVTVSS | | KR | |
| 198 | EVQLLESGGGLVQPGGSLRLS | 2049 | DIQMTQSPSSLSASVGDRVTI | 2056 |
| | CAASGFTFSTFWMSWVRQAP | | TCRASQSISSYLNWYQQKPG | |
| | GKGLEWVATISYDGSNQYYA | | KAPKLLIYDASNLETGVPSRF | |
| | DSVKGRFTISRDNSKNTLYLQ | | SGSGSGTDFTLTISSLQPEDFA | |
| | MNSLRAEDTAVYYCARLELH | | TYYCQQANSFPFTFGPGTKV | |
| | EGRFDYWGQGTLVTVSS | | DIKR | |
| 199 | QVQLVQSGAEVKKPGASVKV | 2050 | DIQMTQSPSSLSASVGDRVTI | 2057 |
| | SCKASGDTFTGYHIHWVRQAP | | TCRASQSVSSWLAWYQQKP | |
| | GQGLEWMGWMNPDSGSTGY | | GKAPKLLIYDASSLQSGVPSR | |
| | AQKFQGRVTMTRDTSTSTVY | | FSGSGSGTDFTLTISSLQPEDF | |
| | MELSSLRSEDTAVYYCTTDRL | | ATYYCQQANSFPFTFGPGTK | |
| | YGDYFDYWGQGTLVTVSS | | VDIKR | |
| 200 | QVQLVQSGAEVKKPGASVKV | 2051 | DIQMTQSPSSLSASVGDRVTI | 2058 |
| | SCKASGYTFTDYYMHWVRQA | | TCQASQDISNYLNWYQQKPG | |
| | PGQGLEWMGWMNPNSGNTG | | KAPKLLIYEASSLQSGVPSRFS | |
| | YAQKFQGRVTMTRDTSTSTV | | GSGSGTDFTLTISSLQPEDFAT | |
| | YMELSSLRSEDTAVYYCAAD | | YYCQQTYSPPPTFGQGTKLEI | |
| | DTKSPYGMDVWGQGTMVTV | | KR | |
| | SS | | | |

The polypeptides above were tested as disclosed above in Example 4. Data is disclosed below in Table 17c, reporting FACS fold change over parental as (−), indicating <2 fold; (+), indicating 2-10 fold; (++), indicating 10-30 fold; and (+++), indicating >30 fold.

TABLE 17c
Polypeptide Activity (FACS)

| | CLL-1 K244 | CLL-1 Q244 |
|---|---|---|
| | BLI/Octet | BLI/Octet |
| | Binding Summary | Binding Summary |
| Polypeptide No. | (Yes/No/Ambiguous) | (Yes/No/Ambiguous) |
| 144 | Yes | Yes |
| 145 | Yes | Yes |
| 146 | Yes | Yes |
| 147 | Yes | Yes |
| 148 | Yes | Yes |
| 149 | Yes | Yes |
| 150 | Yes | Yes |
| 151 | Yes | Yes |
| 152 | Yes | Yes |
| 153 | Yes | Yes |
| 154 | Yes | Yes |
| 155 | Yes | Yes |
| 156 | Yes | Yes |
| 157 | Yes | Yes |
| 158 | Yes | Yes |
| 159 | Yes | Yes |
| 160 | Yes | Yes |
| 161 | Yes | Yes |
| 162 | Yes | Yes |
| 163 | Yes | Yes |
| 164 | Yes | Yes |
| 165 | Yes | Yes |
| 166 | Yes | Yes |
| 167 | Yes | Yes |
| 168 | Yes | Yes |
| 169 | Yes | Yes |
| 170 | Yes | Yes |
| 171 | Yes | Yes |
| 172 | Yes | Yes |
| 173 | Yes | Yes |
| 174 | Yes | Yes |
| 175 | Yes | Yes |
| 176 | Yes | Yes |
| 177 | Yes | Yes |
| 178 | Yes | Yes |
| 179 | Yes | Yes |
| 180 | Yes | Yes |
| 181 | Yes | Yes |
| 182 | Yes | Yes |
| 183 | Yes | Yes |
| 184 | Yes | Yes |
| 185 | Yes | Yes |
| 186 | Yes | Yes |
| 187 | Yes | Yes |
| 188 | Yes | Yes |
| 189 | Yes | Yes |
| 190 | Yes | Yes |
| 194 | Yes | Yes |
| 195 | Yes | Yes |
| 196 | No | Yes |
| 197 | Yes | Yes |
| 198 | Yes | No |
| 199 | Yes | No |
| 200 | Yes | No |

### Example 19: Identification of Non-Selective Anti-Human FLT3 scFv Clones

The methods above in Example 1 have been used to discover non-selective anti-human FLT3 scFv clones. Anti-human FLT-3 scFv clones were discovered by standard screening methodologies of a human antibody library using two recombinant polymorphic forms of human FLT3 extracellular domain antigens (huFLT3-T227 and huFLT3-M227). Using these antigens various panning tactics were employed to encourage enrichment of thermostable clones of desired affinity range. The scFvs were screened for binding to two single nucleotide polymorphism (SNP) variants of human FLT-3 (Threonine 227 and Methionine 227) by flow cytometry and bio-layer interferometry (BLI).

TABLE 18a
Sequences of Non-Selective Anti-FLT3 Polypeptides (CDR Sequences)

| Poly- | | | | | | | | | | | | |
|---|---|---|---|---|---|---|---|---|---|---|---|---|
| peptide | | SEQ ID | | SEQ ID | | SEQ ID | | SEQ ID | | SEQ ID | | SEQ ID |
| No. | HCDR1 | NO | HCDR2 | NO | HCDR3 | NO | LCDR1 | NO | LCDR2 | NO | LCDR3 | NO |
| 201 | GTFSS | 2059 | GWISA | 2153 | CARGG | 2247 | RASHN | 2341 | GATTL | 2435 | CQQAN | 2529 |
| | DGIS | | YHGHT | | KHSGS | | IGNKL | | QS | | SFPRTF | |
| | | | NYA | | HRSYY | | A | | | | | |
| | | | | | YGMD | | | | | | | |
| | | | | | VW | | | | | | | |
| 202 | YTFTN | 2060 | GVINPS | 2154 | CARAG | 2248 | RASQSI | 2342 | DASNL | 2436 | CQQIY | 2530 |
| | YYMH | | GGSTN | | VGAFH | | RSWLA | | ET | | SLPRTF | |
| | | | YA | | IW | | | | | | | |
| 203 | YTLTE | 2061 | GRIIPIS | 2155 | CARAA | 2249 | KSSQS | 2343 | WASTR | 2437 | CQQYY | 2531 |
| | LSMH | | GTANY | | RYCSS | | VLYSS | | AS | | STPQTF | |
| | | | A | | TSCYW | | NNKNY | | | | | |
| | | | | | RDGM | | LA | | | | | |
| | | | | | DVW | | | | | | | |
| 204 | FDFST | 2062 | SGISGS | 2156 | CARVY | 2250 | RASQSI | 2344 | DASSL | 2438 | CQQSY | 2532 |
| | YNMF | | GRSKY | | YDSSG | | GSNLD | | QS | | STPYTF | |
| | | | YA | | YYPYY | | | | | | | |
| | | | | | FDYW | | | | | | | |
| 205 | GSFISH | 2063 | GGIIPIS | 2157 | CAKGR | 2251 | RSSQS | 2345 | AASSL | 2439 | CMQAL | 2533 |
| | TFS | | GTANY | | GQLVG | | LLHSN | | QS | | QTPLTF | |
| | | | A | | GYFQH | | GYNYL | | | | | |
| | | | | | W | | D | | | | | |
| 206 | YTFTS | 2064 | GGIIPIF | 2158 | CARED | 2252 | RASQA | 2346 | AASNL | 2440 | CQQSY | 2534 |
| | YYLH | | GKAEY | | FWSGP | | ISSYLA | | QG | | STPLTF | |
| | | | S | | YGMD | | | | | | | |
| | | | | | VW | | | | | | | |
| 207 | YTFTN | 2065 | GWMN | 2159 | CAKSH | 2253 | QASHD | 2347 | AASIL | 2441 | CQQSY | 2535 |
| | YYMH | | PNSGN | | YYYFY | | ISKYLN | | QS | | STPYTF | |
| | | | TGYA | | GMDV | | | | | | | |
| | | | | | W | | | | | | | |
| 208 | GTFSS | 2066 | GIINPS | 2160 | CAKD | 2254 | RASQS | 2348 | DVSSR | 2442 | CQQYA | 2536 |
| | RSIS | | GGGTL | | MDGW | | LSNIYL | | AA | | TSPLTF | |
| | | | YA | | SDAFDI | | A | | | | | |
| | | | | | W | | | | | | | |
| 209 | FAFSS | 2067 | AGIWV | 2161 | CAREF | 2255 | RASQSI | 2349 | DASKL | 2443 | CQQSY | 2537 |
| | YVLH | | DGHNK | | GAAGS | | STWLA | | ET | | TTPYTF | |
| | | | DYA | | FQHW | | | | | | | |
| 210 | YTLTE | 2068 | GGIIPIS | 2162 | CARAS | 2256 | KSSQS | 2350 | WASTR | 2444 | CHQYY | 2538 |
| | LSMH | | GTTKY | | PRYYM | | VFYSS | | AS | | SKPPTF | |
| | | | A | | DVW | | NNKNY | | | | | |
| | | | | | | | LA | | | | | |
| 211 | FTFSN | 2069 | SAIGA | 2163 | CAKSS | 2257 | RASQG | 2351 | AASTL | 2445 | CQQYG | 2539 |
| | HYMS | | GGGTY | | GYSYG | | ISNNLA | | HN | | RSPKT | |
| | | | YA | | RRPFD | | | | | | F | |
| | | | | | YW | | | | | | | |
| 212 | YTFTG | 2070 | GGIIPIL | 2164 | CARAP | 2258 | RASQS | 2352 | DTSTR | 2446 | CQQYD | 2540 |
| | YYMH | | GTANN | | WGTFD | | VSSSQ | | AT | | NSLWT | |
| | | | A | | YW | | LA | | | | F | |
| 213 | YTFSR | 2071 | GWMN | 2165 | CARTR | 2259 | RASQSI | 2353 | DASNL | 2447 | CQQSY | 2541 |
| | YYMS | | PNSGN | | FAAQP | | SSYLN | | KT | | STPLTF | |
| | | | TGYA | | HNWH | | | | | | | |
| | | | | | FDLW | | | | | | | |
| 214 | YTFTT | 2072 | GIINPS | 2166 | CARDR | 2260 | RASQG | 2354 | AASSL | 2448 | CQQSY | 2542 |
| | YYMH | | GGSTS | | AARPR | | IRNHL | | QS | | STPTF | |
| | | | YA | | GGFDY | | A | | | | | |
| | | | | | W | | | | | | | |
| 215 | YTFTG | 2073 | GWINP | 2167 | CAKGG | 2261 | RASQG | 2355 | AASTL | 2449 | CQQSY | 2543 |
| | YRMH | | NSGGT | | LDWR | | ISSYLA | | QS | | STPLTF | |
| | | | NYA | | NWYF | | | | | | | |
| | | | | | DLW | | | | | | | |
| 216 | NTFTM | 2074 | GAIIPIS | 2168 | CARLS | 2262 | RSSQS | 2356 | LGSNR | 2450 | CMQAL | 2544 |
| | YYMH | | GTVIY | | GGRM | | LLHSN | | AS | | QTPLTF | |
| | | | A | | YDAFD | | GYNYL | | | | | |
| | | | | | IW | | D | | | | | |
| 217 | YTFTTF | 2075 | GGIIPM | 2169 | CASGG | 2263 | KSSQS | 2357 | WASTR | 2451 | CQQYY | 2545 |
| | YLH | | SGTAN | | ENGM | | VLYSV | | ES | | SAPPTF | |
| | | | YA | | DVW | | NNKNY | | | | | |
| | | | | | | | LA | | | | | |
| 218 | YSFTT | 2076 | GIINPS | 2170 | CARGS | 2264 | RASQSI | 2358 | AASSL | 2452 | CQQAI | 2546 |
| | HYMH | | GGSTR | | SYYYY | | STWLA | | QS | | SFPLTF | |
| | | | YA | | GVDV | | | | | | | |
| | | | | | W | | | | | | | |
| 219 | YTFTN | 2077 | GIINPS | 2171 | CARDR | 2265 | RASQG | 2359 | KASSL | 2453 | CLQHN | 2547 |
| | YYMH | | SGSAS | | STLVP | | IRSELS | | ES | | SYPLTF | |
| | | | YT | | LDYW | | | | | | | |
| 220 | YTFTG | 2078 | GIINPR | 2172 | CARGS | 2266 | RASQG | 2360 | AASSL | 2454 | CQQAN | 2548 |
| | YYMY | | GGITS | | TSSGW | | IRNDL | | QS | | RFPPTF | |
| | | | YA | | PNGDM | | G | | | | | |
| | | | | | DVW | | | | | | | |
| 221 | YTFTD | 2079 | GWMT | 2173 | CALGD | 2267 | RSSQS | 2361 | GASYL | 2455 | CMQAL | 2549 |
| | NYMH | | PDSNN | | GPFGM | | LLHSN | | QS | | QGPITF | |
| | | | TGFA | | DVW | | GYNYL | | | | | |
| | | | | | | | D | | | | | |
| 222 | YSFTA | 2080 | GIINPS | 2174 | CARVA | 2268 | RASQD | 2362 | AASTL | 2456 | CQQSY | 2550 |
| | YYMH | | GGSTS | | GINGE | | ISNYLA | | QS | | RTPYT | |
| | | | YA | | MAYW | | | | | | F | |
| 223 | YTFTN | 2081 | GIINPS | 2175 | CAKAL | 2269 | RASQG | 2363 | DGSNL | 2457 | CQQSY | 2551 |
| | SFIH | | GGSTS | | ERRYY | | ISNYLA | | ET | | STPLTF | |
| | | | YA | | YGMD | | | | | | | |
| | | | | | VW | | | | | | | |
| 224 | YTFTG | 2082 | GVINPI | 2176 | CAKAI | 2270 | RASQS | 2364 | DTSSR | 2458 | CQQYA | 2552 |
| | YYMH | | YGTAN | | SSGWS | | VSSDF | | AS | | GPPTF | |
| | | | YA | | NDAFD | | LA | | | | | |
| | | | | | IW | | | | | | | |
| 225 | YTLTE | 2083 | GGIVP | 2177 | CARRR | 2271 | KSSQS | 2365 | SSSTRE | 2459 | CQQYY | 2553 |
| | LSIH | | MSGTA | | DGYNS | | LLYGS | | S | | TTPYTF | |
| | | | SYA | | W | | KNYIS | | | | | |
| 226 | YTFTG | 2084 | GIIDPS | 2178 | CARDR | 2272 | RASQG | 2366 | GASNL | 2460 | CQQSY | 2554 |
| | YYMH | | GGSTS | | SLLWS | | ISNYLA | | QS | | GTPYT | |
| | | | YA | | GVGG | | | | | | F | |
| | | | | | MDVW | | | | | | | |
| 227 | YTLNE | 2085 | GGIIPM | 2179 | CAKGV | 2273 | RASQS | 2367 | GASNL | 2461 | CQQSY | 2555 |
| | LFMH | | SGTTF | | RQYSY | | VSSYL | | QS | | TTPWT | |
| | | | YA | | GRYYY | | N | | | | F | |
| | | | | | GMDV | | | | | | | |
| | | | | | W | | | | | | | |
| 228 | GTFSS | 2086 | GWINP | 2180 | CAKDS | 2274 | RASQSI | 2368 | AASRL | 2462 | CQQSY | 2556 |
| | HAIS | | GSGGT | | YDFWS | | YTHLN | | QT | | SFPFTF | |
| | | | NYA | | GYYID | | | | | | | |
| | | | | | YW | | | | | | | |
| 229 | YTFTN | 2087 | GIINPS | 2181 | CARGV | 2275 | RASQG | 2369 | AASSL | 2463 | CQQSH | 2557 |
| | YYMH | | GGSTS | | GYSGY | | ISNSLA | | QS | | SPPYTF | |
| | | | YA | | GADL | | | | | | | |
| | | | | | W | | | | | | | |
| 230 | GTFSS | 2088 | GGIIPL | 2182 | CARVL | 2276 | RASQS | 2370 | DVSTR | 2464 | CQHYG | 2558 |
| | NAIN | | FGTTN | | SGWY | | VSADY | | AS | | SSQVT | |
| | | | YA | | GTYYF | | IA | | | | F | |
| | | | | | DYW | | | | | | | |
| 231 | YTFTS | 2089 | GLINPS | 2183 | CARGL | 2277 | RASQSI | 2371 | AASNL | 2465 | CQQSY | 2559 |
| | YYIH | | GGSTT | | GWGV | | STYLN | | QS | | SSPLTF | |
| | | | YA | | VVPAA | | | | | | | |
| | | | | | ELDYW | | | | | | | |
| 232 | YTFTS | 2090 | GGIIPIF | 2184 | CTRSN | 2278 | RASQN | 2372 | AASSL | 2466 | CQQYS | 2560 |
| | YGIH | | GTASY | | GIAAA | | IANSLN | | QS | | SYPPTF | |
| | | | A | | GTHW | | | | | | | |
| | | | | | YFDLW | | | | | | | |
| 233 | YTFTS | 2091 | GVINPS | 2185 | CARGI | 2279 | RSSQS | 2373 | AASSL | 2467 | CMQGL | 2561 |
| | | | GGSTT | | GYGGY | | LLHSN | | | | RTPHT | |
| | YYLH | | YA | | FDYW | | GYNYL | | QS | | F | |
| | | | | | | | D | | | | | |
| 234 | HTFTA | 2092 | GWMS | 2186 | CARAT | 2280 | KSSQS | 2374 | WASIR | 2468 | CQQYY | 2562 |
| | YYMH | | PYSGN | | RGTIQ | | VLYSS | | ES | | TTPITF | |
| | | | TGYA | | HW | | NNKNY | | | | | |
| | | | | | | | LA | | | | | |
| 235 | YTFTG | 2093 | GIINPS | 2187 | CARDP | 2281 | RASQS | 2375 | GASTR | 2469 | CQQYG | 2563 |
| | YYMH | | GGSTS | | GRLGE | | VSSNL | | AT | | SSPLTF | |
| | | | YA | | LDYW | | A | | | | | |
| 236 | GTFSS | 2094 | GGIIPIL | 2188 | CAHVD | 2282 | RASQS | 2376 | DVSSR | 2470 | CQQLD | 2564 |
| | YAIS | | GIANY | | GYGM | | VSSNL | | AT | | AYPLT | |
| | | | A | | DVW | | A | | | | F | |
| 237 | YTFTS | 2095 | GLINPS | 2189 | CARSG | 2283 | RSSQS | 2377 | AASTL | 2471 | CMQAL | 2565 |
| | YYMH | | SGSTS | | SGGSY | | LLHSN | | QS | | QTPLTF | |
| | | | YA | | FLFDY | | GYNYL | | | | | |
| | | | | | W | | D | | | | | |
| 238 | YTFTIY | 2096 | GIINPS | 2190 | CARGI | 2284 | RSSQS | 2378 | LGSNR | 2472 | CMQGL | 2566 |
| | YMH | | GGSTV | | GSKGA | | LLHSN | | AS | | QTPYT | |
| | | | YA | | FDIW | | GYNYL | | | | F | |
| | | | | | | | D | | | | | |
| 239 | GTFSS | 2097 | GGIIPIL | 2191 | CARTM | 2285 | RASQS | 2379 | DVSTR | 2473 | CHQYG | 2567 |
| | YAIS | | GTANY | | TTVTY | | VGSSY | | AA | | SSPYTF | |
| | | | A | | YDAFD | | LA | | | | | |
| | | | | | IW | | | | | | | |
| 240 | YTFTS | 2098 | GIINPS | 2192 | CARGL | 2286 | RASQSI | 2380 | AASSL | 2474 | CQQSY | 2568 |
| | YYMH | | SGSTT | | GKSAI | | SSYLN | | QS | | STPLTF | |
| | | | YA | | DYW | | | | | | | |
| 241 | YTFTR | 2099 | GIINPS | 2193 | CARSY | 2287 | RASQSI | 2381 | AASSL | 2475 | CQQSY | 2569 |
| | HYVH | | GGSTS | | HHYYY | | SNYLA | | QG | | STPWT | |
| | | | YA | | GMDV | | | | | | F | |
| | | | | | W | | | | | | | |
| 242 | GTFSS | 2100 | GGIIPM | 2194 | CARDA | 2288 | RASES | 2382 | GASTR | 2476 | CQQYG | 2570 |
| | ATIS | | FGTAN | | YGDST | | VSSAL | | AT | | NSVTF | |
| | | | YA | | W | | A | | | | | |
| 243 | GTFSS | 2101 | GGISP | 2195 | CARAP | 2289 | RASQT | 2383 | DTSSR | 2477 | CHHYG | 2571 |
| | HAFN | | MFGTP | | DYGDD | | LTGGL | | AA | | SSPYTF | |
| | | | NYA | | WYFDL | | LA | | | | | |
| | | | | | W | | | | | | | |
| 244 | YTFTS | 2102 | GRINPS | 2196 | CARVP | 2290 | RASQD | 2384 | AASSL | 2478 | CQQSY | 2572 |
| | YYMH | | GGSTS | | GLYGG | | IRNDL | | QS | | SSPFTF | |
| | | | YA | | AIDYW | | G | | | | | |
| 245 | YTFTG | 2103 | GGVIPF | 2197 | CAYGA | 2291 | RASQS | 2385 | GASTR | 2479 | CQQYS | 2573 |
| | FYIH | | FSRTIY | | NGHLY | | VSSSY | | AT | | SSPLTF | |
| | | | A | | GMDA | | LA | | | | | |
| | | | | | W | | | | | | | |
| 246 | GTFTS | 2104 | GGIIPM | 2198 | CAAGL | 2292 | QATQD | 2386 | GASNL | 2480 | CQQSY | 2574 |
| | YFMH | | FGAPV | | DFWSG | | ISNYLN | | PS | | SDLLTF | |
| | | | YA | | PDNYY | | | | | | | |
| | | | | | MDVW | | | | | | | |
| 247 | GTLMS | 2105 | GIINPR | 2199 | CARSE | 2293 | RGSQSI | 2387 | DTSAR | 2481 | CQQYN | 2575 |
| | YAIS | | GGTTR | | DSGYD | | SGNYL | | AA | | SYPLTF | |
| | | | YA | | YLDY | | A | | | | | |
| | | | | | W | | | | | | | |
| 248 | YTFTG | 2106 | GVINP | 2200 | CAREG | 2294 | RASQD | 2388 | AASSL | 2482 | CQQYY | 2576 |
| | YYMH | | NGGSIS | | WFGED | | LDRYL | | QT | | STPYTF | |
| | | | YA | | GMDV | | A | | | | | |
| | | | | | W | | | | | | | |
| 249 | YTFTS | 2107 | GWMN | 2201 | CATAV | 2295 | RASQSI | 2389 | DTSAH | 2483 | CQHYG | 2577 |
| | DGIS | | PNSGN | | AGTDA | | GNNLK | | TT | | NSLTF | |
| | | | AGYA | | FDIW | | A | | | | | |
| 250 | YTLTSF | 2108 | GRIIPM | 2202 | CASTS | 2296 | RASQS | 2390 | GASTR | 2484 | CQQYG | 2578 |
| | AMH | | SGTAN | | PDQYY | | VGSSS | | AT | | SSPYTF | |
| | | | YA | | YGMD | | LA | | | | | |
| | | | | | VW | | | | | | | |
| 251 | GTFSS | 2109 | GGIIPI | 2203 | CAKGL | 2297 | RASQS | 2391 | DVSTR | 2485 | CQQYG | 2579 |
| | DAIN | | VGTPT | | AFGVF | | VSSNY | | AT | | SSTLTF | |
| | | | YA | | DGLDV | | LA | | | | | |
| | | | | | W | | | | | | | |
| 252 | YTLTD | 2110 | GGIIPM | 2204 | CARSS | 2298 | RASQSI | 2392 | AASSL | 2486 | CQQSY | 2580 |
| | LSIH | | SGTAN | | SSWPK | | SSYLN | | QS | | STPLTF | |
| | | | YA | | YFQH | | | | | | | |
| | | | | | W | | | | | | | |
| 253 | YTFTT | 2111 | GGIVP | 2205 | CASSA | 2299 | RASQD | 2393 | AASSL | 2487 | CQQYD | 2581 |
| | YFMH | | VFGTT | | VGWF | | ISRWL | | QS | | NFPLTF | |
| | | | KYA | | DPW | | A | | | | | |
| 254 | YTFTS | 2112 | GWISP | 2206 | CARGE | 2300 | RASQS | 2394 | DTSTR | 2488 | CQQYG | 2582 |
| | HYMH | | YNGNT | | SNSGW | | VSSSSL | | AT | | TSPITF | |
| | | | NYA | | INFDY | | A | | | | | |
| | | | | | W | | | | | | | |
| 255 | YTLTE | 2113 | GGIIPIS | 2207 | CANKG | 2301 | QASHD | 2395 | ATSSL | 2489 | CQQSY | 2583 |
| | LSMH | | GTVTY | | QQLVR | | IRNSV | | QS | | NTPFTF | |
| | | | A | | GYFQH | | N | | | | | |
| | | | | | W | | | | | | | |
| 256 | YTFAT | 2114 | GMINP | 2208 | CARSS | 2302 | RASHD | 2396 | DASNL | 2490 | CQQAD | 2584 |
| | YYLH | | SGGSTI | | GYDFF | | INNYL | | ET | | SFPLTF | |
| | | | YA | | DYW | | N | | | | | |
| 257 | YTFTN | 2115 | GIINPS | 2209 | CARAH | 2303 | RASQSI | 2397 | AASTL | 2491 | CQQSY | 2585 |
| | YFMH | | GGSTS | | TVYYY | | SSWLA | | QS | | STPWT | |
| | | | YA | | GMDV | | | | | | F | |
| | | | | | W | | | | | | | |
| 258 | GTFGS | 2116 | GWINP | 2210 | CARVG | 2304 | RASQSI | 2398 | SASNL | 2492 | CQQYN | 2586 |
| | YAIS | | NTGGA | | AAAGY | | KGALA | | QS | | SYPLTF | |
| | | | HYA | | QHW | | | | | | | |
| 259 | YTFTSS | 2117 | GGIHP | 2211 | CARAR | 2305 | KSSQS | 2399 | WASTR | 2493 | CQQYY | 2587 |
| | EIN | | MFGTT | | LMVY | | LFYSS | | ES | | SIPYTF | |
| | | | NYA | | APSDY | | NNRNY | | | | | |
| | | | | | W | | LA | | | | | |
| 260 | YTFTN | 2118 | GMINP | 2212 | CARVS | 2306 | RSSQSI | 2400 | GASNL | 2494 | CQQVI | 2588 |
| | YYVH | | SGGST | | GWKR | | STYLN | | QS | | SYPITF | |
| | | | NYA | | GWFDP | | | | | | | |
| | | | | | W | | | | | | | |
| 261 | YTFTR | 2119 | GIINPS | 2213 | CARDL | 2307 | KSSQSI | 2401 | WASTR | 2495 | CQQYY | 2589 |
| | YYMH | | GGSAS | | GGAAA | | SHSPN | | ES | | SSPFTF | |
| | | | YA | | GYFDY | | TRDYL | | | | | |
| | | | | | W | | A | | | | | |
| 262 | FTFSD | 2120 | GWMD | 2214 | CAKDI | 2308 | RASQR | 2402 | DVSAR | 2496 | CQQYL | 2590 |
| | YGYY | | PSSGH | | GWGA | | VGNTY | | AS | | SPPLTF | |
| | MH | | TGYA | | FDIW | | LA | | | | | |
| 263 | GTFSS | 2121 | GGIIPI | 2215 | CAKDI | 2309 | RASQS | 2403 | DVSTR | 2497 | CQQYG | 2591 |
| | YAIS | | VGVAN | | GGYPS | | VSSSY | | AT | | SSPITF | |
| | | | YA | | DAFDI | | LA | | | | | |
| | | | | | W | | | | | | | |
| 264 | YTLTE | 2122 | GGIIPIS | 2216 | CARGA | 2310 | RASQD | 2404 | AASSL | 2498 | CQQYY | 2592 |
| | LSMH | | SATSIP | | LYSSSP | | ISNYLA | | QS | | SYPLTF | |
| | | | | | VRVVA | | | | | | | |
| | | | | | GTKG | | | | | | | |
| | | | | | WFDP | | | | | | | |
| | | | | | W | | | | | | | |
| 265 | HTFTS | 2123 | GRIIPIF | 2217 | CARDD | 2311 | RASQS | 2405 | DTSSR | 2499 | CQQYG | 2593 |
| | DYMH | | GTADY | | SSGIFD | | VNSEH | | AT | | SSPVTF | |
| | | | A | | YW | | LA | | | | | |
| 266 | YSLTE | 2124 | GGINPI | 2218 | CARGT | 2312 | RASQS | 2406 | GASTR | 2500 | CQQSF | 2594 |
| | LSIH | | SGTAN | | VRLN | | VGSQL | | AT | | STPLTF | |
| | | | YA | | WFDP | | G | | | | | |
| | | | | | W | | | | | | | |
| 267 | YTLTSF | 2125 | GMIIPL | 2219 | CANLY | 2313 | RASQD | 2407 | AASSL | 2501 | CQQSF | 2595 |
| | GIS | | SGTTH | | GGNAY | | ISNFVA | | QS | | DTPYT | |
| | | | YA | | YYYG | | | | | | F | |
| | | | | | MDVW | | | | | | | |
| 268 | GTFST | 2126 | GGVIP | 2220 | CASMII | 2314 | RASQS | 2408 | DTSSR | 2502 | CQQYD | 2596 |
| | YALS | | VFGTT | | FGAGG | | VNNNQ | | AT | | TSPYTF | |
| | | | DYA | | WDAY | | LA | | | | | |
| | | | | | YFQEW | | | | | | | |
| 269 | GSFSS | 2127 | GLINPS | 2221 | CARDE | 2315 | RASQSI | 2409 | DVSAR | 2503 | CQQYY | 2597 |
| | YALH | | GGRTS | | GYATF | | SSSYL | | AT | | STPLTF | |
| | | | YA | | DYW | | A | | | | | |
| 270 | GTFSS | 2128 | GWISA | 2222 | CAKD | 2316 | RASQSI | 2410 | DASNL | 2504 | CQQTY | 2598 |
| | YYMH | | YNGNT | | MGYY | | SSYLN | | ET | | TTPLTF | |
| | | | NYA | | YDSSG | | | | | | | |
| | | | | | GFDY | | | | | | | |
| | | | | | W | | | | | | | |
| 271 | GTFSS | 2129 | GGIIPIF | 2223 | CARDL | 2317 | RASQS | 2411 | DISSRA | 2505 | CQQYG | 2599 |
| | YAIS | | GTANY | | SIGYY | | VSYNQ | | A | | GLPAT | |
| | | | A | | GDAFD | | LA | | | | F | |
| | | | | | IW | | | | | | | |
| 272 | YIFTNY | 2130 | GGIIPIF | 2224 | CARGR | 2318 | QASQY | 2412 | DASSL | 2506 | CQQSY | 2600 |
| | YIQ | | GTVGY | | IGGGN | | ISNYLN | | ES | | STPYTF | |
| | | | A | | DYW | | | | | | | |
| 273 | DTFNS | 2131 | GGIIPS | 2225 | CASVS | 2319 | RASQS | 2413 | DASTR | 2507 | CQQYN | 2601 |
| | YAVN | | FGTPT | | YGSFD | | VSSSSL | | AS | | RLPYT | |
| | | | YA | | YW | | A | | | | F | |
| 274 | YTFTY | 2132 | GRITPI | 2226 | CAKDS | 2320 | KSSQS | 2414 | WASTR | 2508 | CQQYY | 2602 |
| | RYLH | | SGTTN | | GQLAH | | VLYSS | | ES | | KTPLTF | |
| | | | YA | | YGMD | | NNKNY | | | | | |
| | | | | | VW | | LA | | | | | |
| 275 | YTFTS | 2133 | GWMN | 2227 | CARVG | 2321 | RASQSI | 2415 | AASSL | 2509 | CQQSY | 2603 |
| | YYMH | | PYSGN | | SGWYS | | SSYLN | | QS | | STPLTF | |
| | | | TGYA | | DYW | | | | | | | |
| 276 | YTFTR | 2134 | GWLNP | 2228 | CASSSS | 2322 | RASQS | 2416 | DTSTR | 2510 | CQQYH | 2604 |
| | FNIH | | FTGNT | | WYGW | | VSSYL | | AT | | SSPWT | |
| | | | GYA | | FDPW | | A | | | | F | |
| 277 | YTFTG | 2135 | GWIDP | 2229 | CARDV | 2323 | RASQS | 2417 | DISSRA | 2511 | CQQYG | 2605 |
| | | | NSGGT | | DTAM | | VDNLV | | T | | RSPITF | |
| | YYMH | | NYA | | VTDY | | G | | | | | |
| | | | | | W | | | | | | | |
| 278 | YTFTS | 2136 | GIINPS | 2230 | CARSV | 2324 | KSSQS | 2418 | WASTR | 2512 | CQQYY | 2606 |
| | YYMH | | SGSTT | | GATSA | | VLYSS | | ES | | SLPVTF | |
| | | | YA | | FDIW | | NNENY | | | | | |
| | | | | | | | LA | | | | | |
| 279 | YTFTK | 2137 | GIINPS | 2231 | CARGR | 2325 | RASQSI | 2419 | KASSL | 2513 | CQQYY | 2607 |
| | YYMH | | GGSTS | | GYSYG | | SSSLN | | ES | | SYPPTF | |
| | | | YA | | YLDY | | | | | | | |
| | | | | | W | | | | | | | |
| 280 | YTFTR | 2138 | GIINPS | 2232 | CARGE | 2326 | QASQD | 2420 | QASNK | 2514 | CQQSY | 2608 |
| | YYMH | | GGSTS | | TRSYA | | ISNYLN | | DT | | STPPTF | |
| | | | YA | | PYGMD | | | | | | | |
| | | | | | VW | | | | | | | |
| 281 | YTFNS | 2139 | GIINPT | 2233 | CAKDP | 2327 | RASQS | 2421 | DASAR | 2515 | CQQYY | 2609 |
| | YGIS | | GGSTT | | FVMDV | | VSSSY | | AA | | STPYTF | |
| | | | YA | | W | | LA | | | | | |
| 282 | GTFSS | 2140 | GWMN | 2234 | CARDF | 2328 | RASRSI | 2422 | DASSR | 2516 | CQQYY | 2610 |
| | YAIS | | PNSGD | | EGGG | | SDYLA | | AT | | TTPLTF | |
| | | | TGYA | | WFDP | | | | | | | |
| | | | | | W | | | | | | | |
| 283 | YTFTS | 2141 | GRINPS | 2235 | CARTP | 2329 | RASQSI | 2423 | AASSL | 2517 | CQQSY | 2611 |
| | YYMD | | SGSTT | | SGSYS | | SSYLN | | QS | | STPWT | |
| | | | YV | | DFDY | | | | | | F | |
| | | | | | W | | | | | | | |
| 284 | YTFTS | 2142 | GVINPS | 2236 | CARVP | 2330 | RASHN | 2424 | AASSL | 2518 | CQQSY | 2612 |
| | YYMH | | GGSTT | | GVSPG | | ISTWL | | QS | | STPPTF | |
| | | | YA | | DYGM | | A | | | | | |
| | | | | | DVW | | | | | | | |
| 285 | YSFTN | 2143 | GGIIPV | 2237 | CARES | 2331 | KSSQS | 2425 | WASTR | 2519 | CQQYY | 2613 |
| | YYMH | | FGTTT | | QDGDF | | VLYSS | | ES | | SSPLTF | |
| | | | YS | | DYW | | NNKNY | | | | | |
| | | | | | | | LA | | | | | |
| 286 | YTFTS | 2144 | GWISP | 2238 | CVSDD | 2332 | RASQS | 2426 | DVSTR | 2520 | CQQYN | 2614 |
| | YGIS | | NSGVT | | YGAFD | | VSSSY | | AS | | NWPYT | |
| | | | NYA | | YW | | LA | | | | F | |
| 287 | YTFTR | 2145 | GIINPS | 2239 | CARDR | 2333 | RASQSI | 2427 | AASSL | 2521 | CQQSY | 2615 |
| | HYVH | | SGSAS | | LRSRF | | SSSLA | | QS | | TIPPTF | |
| | | | YA | | DYW | | | | | | | |
| 288 | YTFTT | 2146 | GWMN | 2240 | CARED | 2334 | RASQG | 2428 | KASTL | 2522 | CQQSY | 2616 |
| | YDIN | | PSSGN | | YYDSS | | ISNNLN | | ES | | STPITF | |
| | | | SGFA | | GYYN | | | | | | | |
| | | | | | W | | | | | | | |
| 289 | YTFTS | 2147 | GWMN | 2241 | CVVER | 2335 | QASQG | 2429 | KASSL | 2523 | CQQGY | 2617 |
| | YGIS | | PISGNT | | RREVG | | ITSYLN | | ES | | STPLTF | |
| | | | DYA | | MDVW | | | | | | | |
| 290 | GTFTS | 2148 | GWISA | 2242 | CARDQ | 2336 | RASQSI | 2430 | DASNL | 2524 | CQQTY | 2618 |
| | YYMH | | YNGKT | | GYYYD | | SSYLN | | ET | | SAPPTF | |
| | | | DYA | | SSGAF | | | | | | | |
| | | | | | DIW | | | | | | | |
| 291 | YTFTS | 2149 | GIINPS | 2243 | CARGI | 2337 | RSSQS | 2431 | LGSNR | 2525 | CMQGL | 2619 |
| | YYMH | | GGSTV | | GSKGA | | LLHSN | | AS | | QTPYT | |
| | | | YA | | FDIW | | GYNYL | | | | F | |
| | | | | | | | D | | | | | |
| 292 | YTFTS | 2150 | GWINP | 2244 | CARQG | 2338 | RASQSI | 2432 | DTSSL | 2526 | CQQSFI | 2620 |
| | YGIS | | NSGGT | | GLRDF | | STYVN | | QS | | TPPTF | |
| | | | NYA | | DYW | | | | | | | |
| 293 | YMFTT | 2151 | GVINPI | 2245 | CANDR | 2339 | RSSQS | 2433 | LGSNR | 2527 | CMQAL | 2621 |
| | PYIH | | SGTTT | | HYDF | | LLHSN | | AS | | QTPTF | |
| | | | YA | | WSGY | | GYNYL | | | | | |
| | | | | | YKEEW | | D | | | | | |
| | | | | | EYFQH | | | | | | | |
| | | | | | W | | | | | | | |
| 294 | YTFTS | 2152 | GWINL | 2246 | CAKAI | 2340 | RASQG | 2434 | QASSL | 2528 | CQQAY | 2622 |
| | NNMH | | NSGGT | | DYYY | | IRNDL | | EN | | SLPWT | |
| | | | NYA | | MDVW | | G | | | | F | |

TABLE 18b
Sequences of Non-Selective Anti-FLT3 Polypeptides (VH and VL
Sequences)

| Polypeptide | | SEQ ID | | SEQ ID |
|---|---|---|---|---|
| No. | Full VH | NO | Full VL | NO |
| 201 | QVQLVQSGAEVKKPGASVKV | 2623 | DIQMTQSPSSLSASVGDRVTI | 2717 |
| | SCKASGGTFSSDGISWVRQAP | | TCRASHNIGNKLAWYQQKPG | |
| | GQGLEWMGWISAYHGHTNY | | KAPKLLIYGATTLQSGVPSRF | |
| | AQKFQGRVTMTRDTSTSTVY | | SGSGSGTDFTLTISSLQPEDFA | |
| | MELSSLRSEDTAVYYCARGG | | TYYCQQANSFPRTFGPGTKV | |
| | KHSGSHRSYYYGMDVWGQG | | DIKR | |
| | TTVTVSS | | | |
| 202 | QVQLVQSGAEVKKPGASVKV | 2624 | DIQMTQSPSSLSASVGDRVTI | 2718 |
| | SCKASGYTFTNYYMHWVRQA | | TCRASQSIRSWLAWYQQKPG | |
| | PGQGLEWMGVINPSGGSTNY | | KAPKLLIYDASNLETGVPSRF | |
| | AQKFQGRVTMTRDTSTSTVY | | SGSGSGTDFTLTISSLQPEDFA | |
| | MELSSLRSEDTAVYYCARAG | | TYYCQQIYSLPRTFGQGTKVE | |
| | VGAFHIWGQGTMVTVSS | | IKR | |
| 203 | QVQLVQSGAEVKKPGSSVKV | 2625 | DIVMTQSPDSLAVSLGERATI | 2719 |
| | SCKASGYTLTELSMHWVRQA | | NCKSSQSVLYSSNNKNYLAW | |
| | PGQGLEWMGRIIPISGTANYA | | YQQKPGQPPKLLIYWASTRA | |
| | QKFQGRVTITADESTSTAYME | | SGVPDRFSGSGSGTDFTLTISS | |
| | LSSLRSEDTAVYYCARAARYC | | LQAEDVAVYYCQQYYSTPQT | |
| | SSTSCYWRDGMDVWGQGTT | | FGQGTKLEIKR | |
| | VTVSS | | | |
| 204 | EVQLLESGGGLVQPGGSLRLS | 2626 | DIQMTQSPSSLSASVGDRVTI | 2720 |
| | CAASGFDFSTYNMFWVRQAP | | TCRASQSIGSNLDWYQQKPG | |
| | GKGLEWVSGISGSGRSKYYA | | KAPKLLIYDASSLQSGVPSRF | |
| | DSVKGRFTISRDNSKNTLYLQ | | SGSGSGTDFTLTISSLQPEDFA | |
| | MNSLRAEDTAVYYCARVYYD | | TYYCQQSYSTPYTFGQGTKL | |
| | SSGYYPYYFDYWGQGTLVTV | | EIKR | |
| | SS | | | |
| 205 | QVQLVQSGAEVKKPGSSVKV | 2627 | DIVMTQSPLSLPVTPGEPASIS | 2721 |
| | SCKASGGSFISHTFSWVRQAP | | CRSSQSLLHSNGYNYLDWYL | |
| | GQGLEWMGGIIPISGTANYAQ | | QKPGQSPQLLIYAASSLQSGV | |
| | KFQGRVTITADESTSTAYMEL | | PDRFSGSGSGTDFTLKISRVE | |
| | SSLRSEDTAVYYCAKGRGQL | | AEDVGVYYCMQALQTPLTFG | |
| | VGGYFQHWGQGTLVTVSS | | QGTRLEIKR | |
| 206 | QVQLVQSGAEVKKPGSSVKV | 2628 | DIQMTQSPSSLSASVGDRVTI | 2722 |
| | SCKASGYTFTSYYLHWVRQA | | TCRASQAISSYLAWYQQKPG | |
| | PGQGLEWMGGIIPIFGKAEYS | | KAPKLLIYAASNLQGGVPSRF | |
| | QRFQGRVTITADESTSTAYME | | SGSGSGTDFTLTISSLQPEDFA | |
| | LSSLRSEDTAVYYCAREDFWS | | TYYCQQSYSTPLTFGGGTKV | |
| | GPYGMDVWGQGTTVTVSS | | EIKR | |
| 207 | QVQLVQSGAEVKKPGASVKV | 2629 | DIQMTQSPSSLSASVGDRVTI | 2723 |
| | SCKASGYTFTNYYMHWVRQA | | TCQASHDISKYLNWYQQKPG | |
| | PGQGLEWMGWMNPNSGNTG | | KAPKLLIYAASILQSGVPSRFS | |
| | YAQRFQGRVTMTRDTSTSTV | | GSGSGTDFTLTISSLQPEDFAT | |
| | YMELSSLRSEDTAVYYCAKSH | | YYCQQSYSTPYTFGQGTKLEI | |
| | YYYFYGMDVWGQGTTVTVSS | | KR | |
| 208 | QVQLVQSGAEVKKPGASVKV | 2630 | EIVMTQSPATLSVSPGERATL | 2724 |
| | SCKASGGTFSSRSISWVRQAP | | SCRASQSLSNIYLAWYQQKP | |
| | GQGLEWMGIINPSGGGTLYAQ | | GQAPRLLIYDVSSRAAGIPAR | |
| | KFQGRVTMTRDTSTSTVYME | | FSGSGSGTEFTLTISSLQSEDF | |
| | LSSLRSEDTAVYYCAKDMDG | | AVYYCQQYATSPLTFGGGTK | |
| | WSDAFDIWGQGTTVTVSS | | VEIKR | |
| 209 | EVQLLESGGGLVQPGGSLRLS | 2631 | DIQMTQSPSSLSASVGDRVTI | 2725 |
| | CAASGFAFSSYVLHWVRQAP | | TCRASQSISTWLAWYQQKPG | |
| | GKGLEWVAGIWVDGHNKDY | | KAPKLLIYDASKLETGVPSRF | |
| | ADSVKGRFTISRDNSKNTLYL | | SGSGSGTDFTLTISSLQPEDFA | |
| | QMNSLRAEDTAVYYCAREFG | | TYYCQQSYTTPYTFGQGTKV | |
| | AAGSFQHWGQGTLVTVSS | | EIKR | |
| 210 | QVQLVQSGAEVKKPGSSVKV | 2632 | DIVMTQSPDSLAVSLGERATI | 2726 |
| | SCKASGYTLTELSMHWVRQA | | NCKSSQSVFYSSNNKNYLAW | |
| | PGQGLEWMGGIIPISGTTKYA | | YQQKPGQPPKLLIYWASTRA | |
| | QKFQGRVTITADESTSTAYME | | SGVPDRFSGSGSGTDFTLTISS | |
| | LSSLRSEDTAVYYCARASPRY | | LQAEDVAVYYCHQYYSKPPT | |
| | YMDVWGKGTTVTVSS | | FGQGTKVEIKR | |
| 211 | EVQLLESGGGLVQPGGSLRLS | 2633 | DIQMTQSPSSLSASVGDRVTI | 2727 |
| | CAASGFTFSNHYMSWVRQAP | | TCRASQGISNNLAWYQQKPG | |
| | GKGLEWVSAIGAGGGTYYAD | | KAPKLLIYAASTLHNGVPSRF | |
| | SVKGRFTISRDNSKNTLYLQM | | SGSGSGTDFTLTISSLQPEDFA | |
| | NSLRAEDTAVYYCAKSSGYS | | TYYCQQYGRSPKTFGQGTKV | |
| | YGRRPFDYWGQGTLVTVSS | | EIKR | |
| 212 | QVQLVQSGAEVKKPGSSVKV | 2634 | EIVMTQSPATLSVSPGERATL | 2728 |
| | SCKASGYTFTGYYMHWVRQA | | SCRASQSVSSSQLAWYQQKP | |
| | PGQGLEWMGGIIPILGTANNA | | GQAPRLLIYDTSTRATGIPAR | |
| | QKFQGRVTITADESTSTAYME | | FSGSGSGTEFTLTISSLQSEDF | |
| | LSSLRSEDTAVYYCARAPWGT | | AVYYCQQYDNSLWTFGQGT | |
| | FDYWGQGTLVTVSS | | RLEIKR | |
| 213 | QVQLVQSGAEVKKPGASVKV | 2635 | DIQMTQSPSSLSASVGDRVTI | 2729 |
| | SCKASGYTFSRYYMSWVRQA | | TCRASQSISSYLNWYQQKPG | |
| | PGQGLEWMGWMNPNSGNTG | | KAPKLLIYDASNLKTGVPSRF | |
| | YAQKFQGRVTMTRDTSTSTV | | SGSGSGTDFTLTISSLQPEDFA | |
| | YMELSSLRSEDTAVYYCARTR | | TYYCQQSYSTPLTFGPGTKV | |
| | FAAQPHNWHFDLWGRGTLVT | | DIKR | |
| | VSS | | | |
| 214 | QVQLVQSGAEVKKPGASVKV | 2636 | DIQMTQSPSSLSASVGDRVTI | 2730 |
| | SCKASGYTFTTYYMHWVRQA | | TCRASQGIRNHLAWYQQKPG | |
| | PGQGLEWMGIINPSGGSTSYA | | KAPKLLIYAASSLQSGVPSRF | |
| | QKFQGRVTMTRDTSTSTVYM | | SGSGSGTDFTLTISSLQPEDFA | |
| | ELSSLRSEDTAVYYCARDRAA | | TYYCQQSYSTPTFGQGTKVEI | |
| | RPRGGFDYWGQGTLVTVSS | | KR | |
| 215 | QVQLVQSGAEVKKPGASVKV | 2637 | DIQMTQSPSSLSASVGDRVTI | 2731 |
| | SCKASGYTFTGYRMHWVRQA | | TCRASQGISSYLAWYQQKPG | |
| | PGQGLEWMGWINPNSGGTNY | | KAPKLLIYAASTLQSGVPSRF | |
| | AQKFQGRVTMTRDTSTSTVY | | SGSGSGTDFTLTISSLQPEDFA | |
| | MELSSLRSEDTAVYYCAKGG | | TYYCQQSYSTPLTFGGGTKV | |
| | LDWRNWYFDLWGRGTLVTV | | EIKR | |
| | SS | | | |
| 216 | QVQLVQSGAEVKKPGSSVKV | 2638 | DIVMTQSPLSLPVTPGEPASIS | 2732 |
| | SCKASGNTFTMYYMHWVRQ | | CRSSQSLLHSNGYNYLDWYL | |
| | APGQGLEWVGAIIPISGTVIYA | | QKPGQSPQLLIYLGSNRASGV | |
| | RKFQGRVTITADESTSTAYME | | PDRFSGSGSGTDFTLKISRVE | |
| | LSSLRSEDTAVYYCARLSGGR | | AEDVGVYYCMQALQTPLTFG | |
| | MYDAFDIWGQGTTVTVSS | | QGTKVEIKR | |
| 217 | QVQLVQSGAEVKKPGSSVKV | 2639 | DIVMTQSPDSLAVSLGERATI | 2733 |
| | SCKASGYTFTTFYLHWVRQAP | | NCKSSQSVLYSVNNKNYLA | |
| | GQGLEWIGGIIPMSGTANYAQ | | WYQQKPGQPPKLLIYWASTR | |
| | KFQGRVTITADESTSTAYMEL | | ESGVPDRFSGSGSGTDFTLTIS | |
| | SSLRSEDTAVYYCASGGENG | | SLQAEDVAVYYCQQYYSAPP | |
| | MDVWGQGTTVTVSS | | TFGQGTKVEIKR | |
| 218 | QVQLVQSGAEVKKPGASVKV | 2640 | DIQMTQSPSSLSASVGDRVTI | 2734 |
| | SCKASGYSFTTHYMHWVRQA | | TCRASQSISTWLAWYQQKPG | |
| | PGQGLEWMGIINPSGGSTRYA | | KAPKLLIYAASSLQSGVPSRF | |
| | QKFQGRVTMTRDTSTSTVYM | | SGSGSGTDFTLTISSLQPEDFA | |
| | ELSSLRSEDTAVYYCARGSSY | | TYYCQQAISFPLTFGGGTKVE | |
| | YYYGVDVWGKGTTVTVSS | | IKR | |
| 219 | QVQLVQSGAEVKKPGASVKV | 2641 | DIQMTQSPSSLSASVGDRVTI | 2735 |
| | SCKASGYTFTNYYMHWVRQA | | TCRASQGIRSELSWYQQKPG | |
| | PGQGLEWMGIINPSSGSASYT | | KAPKLLIYKASSLESGVPSRFS | |
| | QKLQGRVTMTRDTSTSTVYM | | GSGSGTDFTLTISSLQPEDFAT | |
| | ELSSLRSEDTAVYYCARDRST | | YYCLQHNSYPLTFGGGTKVEI | |
| | LVPLDYWGQGTLVTVSS | | KR | |
| 220 | QVQLVQSGAEVKKPGASVKV | 2642 | DIQMTQSPSSLSASVGDRVTI | 2736 |
| | SCKASGYTFTGYYMYWVRQA | | TCRASQGIRNDLGWYQQKPG | |
| | PGQGLEWMGIINPRGGITSYA | | KAPKLLIYAASSLQSGVPSRF | |
| | QRFQGRVTMTRDTSTSTVYM | | SGSGSGTDFTLTISSLQPEDFA | |
| | ELSSLRSEDTAVYYCARGSTS | | TYYCQQANRFPPTFGQGTKV | |
| | SGWPNGDMDVWGKGTTVTV | | EIKR | |
| | SS | | | |
| 221 | QVQLVQSGAEVKKPGASVKV | 2643 | DIVMTQSPLSLPVTPGEPASIS | 2737 |
| | SCKASGYTFTDNYMHWVRQA | | CRSSQSLLHSNGYNYLDWYL | |
| | PGQGLEWMGWMTPDSNNTG | | QKPGQSPQLLIYGASYLQSGV | |
| | FAQNFQGRVTMTRDTSTSTVY | | PDRFSGSGSGTDFTLKISRVE | |
| | MELSSLRSEDTAVYYCALGD | | AEDVGVYYCMQALQGPITFG | |
| | GPFGMDVWGQGTTVTVSS | | QGTKVEIKR | |
| 222 | QVQLVQSGAEVKKPGASVKV | 2644 | DIQMTQSPSSLSASVGDRVTI | 2738 |
| | SCKASGYSFTAYYMHWVRQA | | TCRASQDISNYLAWYQQKPG | |
| | PGQGLEWMGIINPSGGSTSYA | | KAPKLLIYAASTLQSGVPSRF | |
| | QKFQGRVTMTRDTSTSTVYM | | SGSGSGTDFTLTISSLQPEDFA | |
| | ELSSLRSEDTAVYYCARVAGI | | TYYCQQSYRTPYTFGQGTKV | |
| | NGEMAYWGQGTLVTVSS | | EIKR | |
| 223 | QVQLVQSGAEVKKPGASVKV | 2645 | DIQMTQSPSSLSASVGDRVTI | 2739 |
| | SCKASGYTFTNSFIHWVRQAP | | TCRASQGISNYLAWYQQKPG | |
| | GQGLEWMGIINPSGGSTSYAQ | | KAPKLLIYDGSNLETGVPSRF | |
| | KFQGRVTMTRDTSTSTVYME | | SGSGSGTDFTLTISSLQPEDFA | |
| | LSSLRSEDTAVYYCAKALERR | | TYYCQQSYSTPLTFGQGTKV | |
| | YYYGMDVWGQGTTVTVSS | | EIKR | |
| 224 | QVQLVQSGAEVKKPGSSVKV | 2646 | EIVMTQSPATLSVSPGERATL | 2740 |
| | SCKASGYTFTGYYMHWVRQA | | SCRASQSVSSDFLAWYQQKP | |
| | PGQGLEWMGVINPIYGTANY | | GQAPRLLIYDTSSRASGIPARF | |
| | ALKFQGRVTITADESTSTAYM | | SGSGSGTEFTLTISSLQSEDFA | |
| | ELSSLRSEDTAVYYCAKAISSG | | VYYCQQYAGPPTFGQGTRLE | |
| | WSNDAFDIWGQGTMVTVSS | | IKR | |
| 225 | QVQLVQSGAEVKKPGSSVKV | 2647 | DIVMTQSPDSLAVSLGERATI | 2741 |
| | SCKASGYTLTELSIHWVRQAP | | NCKSSQSLLYGSKNYISWYQ | |
| | GQGLEWMGGIVPMSGTASYA | | QKPGQPPKLLIYSSSTRESGVP | |
| | QKFQGRVTITADESTSTAYME | | DRFSGSGSGTDFTLTISSLQAE | |
| | LSSLRSEDTAVYYCARRRDGY | | DVAVYYCQQYYTTPYTFGQ | |
| | NSWGQGTLVTVSS | | GTKVEIKR | |
| 226 | QVQLVQSGAEVKKPGASVKV | 2648 | DIQMTQSPSSLSASVGDRVTI | 2742 |
| | SCKASGYTFTGYYMHWVRQA | | TCRASQGISNYLAWYQQKPG | |
| | PGQGLEWMGIIDPSGGSTSYA | | KAPKLLIYGASNLQSGVPSRF | |
| | QKFQGRVTMTRDTSTSTVYM | | SGSGSGTDFTLTISSLQPEDFA | |
| | ELSSLRSEDTAVYYCARDRSL | | TYYCQQSYGTPYTFGQGTKL | |
| | LWSGVGGMDVWGQGTTVTV | | EIKR | |
| | SS | | | |
| 227 | QVQLVQSGAEVKKPGSSVKV | 2649 | DIQMTQSPSSLSASVGDRVTI | 2743 |
| | SCKASGYTLNELFMHWVRQA | | TCRASQSVSSYLNWYQQKPG | |
| | PGQGLEWVGGIIPMSGTTFYA | | KAPKLLIYGASNLQSGVPSRF | |
| | QTFQGRVTITADESTSTAYME | | SGSGSGTDFTLTISSLQPEDFA | |
| | LSSLRSEDTAVYYCAKGVRQ | | TYYCQQSYTTPWTFGQGTRL | |
| | YSYGRYYYGMDVWGQGTLV | | EIKR | |
| | TVSS | | | |
| 228 | QVQLVQSGAEVKKPGASVKV | 2650 | DIQMTQSPSSLSASVGDRVTI | 2744 |
| | SCKASGGTFSSHAISWVRQAP | | TCRASQSIYTHLNWYQQKPG | |
| | GQGLEWMGWINPGSGGTNYA | | KAPKLLIYAASRLQTGVPSRF | |
| | QKFQGRVTMTRDTSTSTVYM | | SGSGSGTDFTLTISSLQPEDFA | |
| | ELSSLRSEDTAVYYCAKDSYD | | TYYCQQSYSFPFTFGPGTKVD | |
| | FWSGYYIDYWGQGTLVTVSS | | IKR | |
| 229 | QVQLVQSGAEVKKPGASVKV | 2651 | DIQMTQSPSSLSASVGDRVTI | 2745 |
| | SCKASGYTFTNYYMHWVRQA | | TCRASQGISNSLAWYQQKPG | |
| | PGQGLEWMGIINPSGGSTSYA | | KAPKLLIYAASSLQSGVPSRF | |
| | QKFQGRVTMTRDTSTSTVYM | | SGSGSGTDFTLTISSLQPEDFA | |
| | ELSSLRSEDTAVYYCARGVGY | | TYYCQQSHSPPYTFGQGTKL | |
| | SGYGADLWGRGTLVTVSS | | EIKR | |
| 230 | QVQLVQSGAEVKKPGSSVKV | 2652 | EIVMTQSPATLSVSPGERATL | 2746 |
| | SCKASGGTFSSNAINWVRQAP | | SCRASQSVSADYIAWYQQKP | |
| | GQGLEWMGGIIPLFGTTNYAQ | | GQAPRLLIYDVSTRASGIPAR | |
| | KFQGRVTITADESTSTAYMEL | | FSGSGSGTEFTLTISSLQSEDF | |
| | SSLRSEDTAVYYCARVLSGW | | AVYYCQHYGSSQVTFGQGTK | |
| | YGTYYFDYWGQGTLVTVSS | | VEIKR | |
| 231 | QVQLVQSGAEVKKPGASVKV | 2653 | DIQMTQSPSSLSASVGDRVTI | 2747 |
| | SCKASGYTFTSYYIHWVRQAP | | TCRASQSISTYLNWYQQKPG | |
| | GQGLEWMGLINPSGGSTTYA | | KAPKLLIYAASNLQSGVPSRF | |
| | QSFQGRVTMTRDTSTSTVYM | | SGSGSGTDFTLTISSLQPEDFA | |
| | ELSSLRSEDTAVYYCARGLG | | TYYCQQSYSSPLTFGPGTKVD | |
| | WGVVVPAAELDYWGQGTLV | | IKR | |
| | TVSS | | | |
| 232 | QVQLVQSGAEVKKPGASVKV | 2654 | DIQMTQSPSSLSASVGDRVTI | 2748 |
| | SCKASGYTFTSYGIHWVRQAP | | TCRASQNIANSLNWYQQKPG | |
| | GQGLEWMGGIIPIFGTASYAQ | | KAPKLLIYAASSLQSGVPSRF | |
| | KFQGRVTMTRDTSTSTVYME | | SGSGSGTDFTLTISSLQPEDFA | |
| | LSSLRSEDTAVYYCTRSNGIA | | TYYCQQYSSYPPTFGPGTKV | |
| | AAGTHWYFDLWGRGTLVTVS | | DIKR | |
| | S | | | |
| 233 | QVQLVQSGAEVKKPGASVKV | 2655 | DIVMTQSPLSLPVTPGEPASIS | 2749 |
| | SCKASGYTFTSYYLHWVRQA | | CRSSQSLLHSNGYNYLDWYL | |
| | PGQGLEWIGVINPSGGSTTYA | | QKPGQSPQLLIYAASSLQSGV | |
| | QRFQGRVTMTRDTSTSTVYM | | PDRFSGSGSGTDFTLKISRVE | |
| | ELSSLRSEDTAVYYCARGIGY | | AEDVGVYYCMQGLRTPHTF | |
| | GGYFDYWGQGTLVTVSS | | GGGTKVEIKR | |
| 234 | QVQLVQSGAEVKKPGSSVKV | 2656 | DIVMTQSPDSLAVSLGERATI | 2750 |
| | SCKASGHTFTAYYMHWVRQA | | NCKSSQSVLYSSNNKNYLAW | |
| | PGQGLEWMGWMSPYSGNTG | | YQQKPGQPPKLLIYWASIRES | |
| | YAQNFQGRVTITADESTSTAY | | GVPDRFSGSGSGTDFTLTISSL | |
| | MELSSLRSEDTAVYYCARATR | | QAEDVAVYYCQQYYTTPITF | |
| | GTIQHWGQGTLVTVSS | | GQGTRLEIKR | |
| 235 | QVQLVQSGAEVKKPGASVKV | 2657 | EIVMTQSPATLSVSPGERATL | 2751 |
| | SCKASGYTFTGYYMHWVRQA | | SCRASQSVSSNLAWYQQKPG | |
| | PGQGLEWMGIINPSGGSTSYA | | QAPRLLIYGASTRATGIPARFS | |
| | QKFQGRVTMTRDTSTSTVYM | | GSGSGTEFTLTISSLQSEDFAV | |
| | ELSSLRSEDTAVYYCARDPGR | | YYCQQYGSSPLTFGGGTKVEI | |
| | LGELDYWGQGTLVTVSS | | KR | |
| 236 | QVQLVQSGAEVKKPGSSVKV | 2658 | EIVMTQSPATLSVSPGERATL | 2752 |
| | SCKASGGTFSSYAISWVRQAP | | SCRASQSVSSNLAWYQQKPG | |
| | GQGLEWMGGIIPILGIANYAQ | | QAPRLLIYDVSSRATGIPARFS | |
| | KFQGRVTITADESTSTAYMEL | | GSGSGTEFTLTISSLQSEDFAV | |
| | SSLRSEDTAVYYCAHVDGYG | | YYCQQLDAYPLTFGGGTKVE | |
| | MDVWGQGTTVTVSS | | IKR | |
| 237 | QVQLVQSGAEVKKPGASVKV | 2659 | DIVMTQSPLSLPVTPGEPASIS | 2753 |
| | SCKASGYTFTSYYMHWVRQA | | CRSSQSLLHSNGYNYLDWYL | |
| | PGQGLEWMGLINPSSGSTSYA | | QKPGQSPQLLIYAASTLQSGV | |
| | RNFQGRVTMTRDTSTSTVYM | | PDRFSGSGSGTDFTLKISRVE | |
| | ELSSLRSEDTAVYYCARSGSG | | AEDVGVYYCMQALQTPLTFG | |
| | GSYFLFDYWGQGTLVTVSS | | GGTKVEIKR | |
| 238 | QVQLVQSGAEVKKPGASVKV | 2660 | DIVMTQSPLSLPVTPGEPASIS | 2754 |
| | SCKASGYTFTIYYMHWVRQA | | CRSSQSLLHSNGYNYLDWYL | |
| | PGQGLEWMGIINPSGGSTVYA | | QKPGQSPQLLIYLGSNRASGV | |
| | QTFQGRVTMTRDTSTSTVYM | | PDRFSGSGSGTDFTLKISRVE | |
| | ELSSLRSEDTAVYYCARGIGS | | AEDVGVYYCMQGLQTPYTF | |
| | KGAFDIWGQGTMVTVSS | | GQGTRLEIKR | |
| 239 | QVQLVQSGAEVKKPGSSVKV | 2661 | EIVMTQSPATLSVSPGERATL | 2755 |
| | SCKASGGTFSSYAISWVRQAP | | SCRASQSVGSSYLAWYQQKP | |
| | GQGLEWMGGIIPILGTANYAQ | | GQAPRLLIYDVSTRAAGIPAR | |
| | KFQGRVTITADESTSTAYMEL | | FSGSGSGTEFTLTISSLQSEDF | |
| | SSLRSEDTAVYYCARTMTTVT | | AVYYCHQYGSSPYTFGQGTK | |
| | YYDAFDIWGQGTMVTVSS | | VEIKR | |
| 240 | QVQLVQSGAEVKKPGASVKV | 2662 | DIQMTQSPSSLSASVGDRVTI | 2756 |
| | SCKASGYTFTSYYMHWVRQA | | TCRASQSISSYLNWYQQKPG | |
| | PGQGLEWMGIINPSSGSTTYA | | KAPKLLIYAASSLQSGVPSRF | |
| | LKFQGRVTMTRDTSTSTVYM | | SGSGSGTDFTLTISSLQPEDFA | |
| | ELSSLRSEDTAVYYCARGLGK | | TYYCQQSYSTPLTFGQGTRLE | |
| | SAIDYWGQGTLVTVSS | | IKR | |
| 241 | QVQLVQSGAEVKKPGASVKV | 2663 | DIQMTQSPSSLSASVGDRVTI | 2757 |
| | SCKASGYTFTRHYVHWVRQA | | TCRASQSISNYLAWYQQKPG | |
| | PGQGLEWMGIINPSGGSTSYA | | KAPKLLIYAASSLQGGVPSRF | |
| | QKFQGRVTMTRDTSTSTVYM | | SGSGSGTDFTLTISSLQPEDFA | |
| | ELSSLRSEDTAVYYCARSYHH | | TYYCQQSYSTPWTFGQGTKV | |
| | YYYGMDVWGQGTTVTVSS | | EIKR | |
| 242 | QVQLVQSGAEVKKPGSSVKV | 2664 | EIVMTQSPATLSVSPGERATL | 2758 |
| | SCKASGGTFSSATISWVRQAP | | SCRASESVSSALAWYQQKPG | |
| | GQGLEWMGGIIPMFGTANYA | | QAPRLLIYGASTRATGIPARFS | |
| | QKFQGRVTITADESTSTAYME | | GSGSGTEFTLTISSLQSEDFAV | |
| | LSSLRSEDTAVYYCARDAYG | | YYCQQYGNSVTFGGGTKVEI | |
| | DSTWGQGTLVTVSS | | KR | |
| 243 | QVQLVQSGAEVKKPGSSVKV | 2665 | EIVMTQSPATLSVSPGERATL | 2759 |
| | SCKASGGTFSSHAFNWVRQAP | | SCRASQTLTGGLLAWYQQKP | |
| | GQGLEWMGGISPMFGTPNYA | | GQAPRLLIYDTSSRAAGIPAR | |
| | QKFQGRVTITADESTSTAYME | | FSGSGSGTEFTLTISSLQSEDF | |
| | LSSLRSEDTAVYYCARAPDYG | | AVYYCHHYGSSPYTFGQGTK | |
| | DDWYFDLWGRGTLVTVSS | | VEIKR | |
| 244 | QVQLVQSGAEVKKPGASVKV | 2666 | DIQMTQSPSSLSASVGDRVTI | 2760 |
| | SCKASGYTFTSYYMHWVRQA | | TCRASQDIRNDLGWYQQKPG | |
| | PGQGLEWMGRINPSGGSTSYA | | KAPKLLIYAASSLQSGVPSRF | |
| | QSFQGRVTMTRDTSTSTVYM | | SGSGSGTDFTLTISSLQPEDFA | |
| | ELSSLRSEDTAVYYCARVPGL | | TYYCQQSYSSPFTFGQGTKVE | |
| | YGGAIDYWGQGTLVTVSS | | IKR | |
| 245 | QVQLVQSGAEVKKPGSSVKV | 2667 | EIVMTQSPATLSVSPGERATL | 2761 |
| | SCKASGYTFTGFYIHWVRQAP | | SCRASQSVSSSYLAWYQQKP | |
| | GQGLEWMGGVIPFFSRTIYAQ | | GQAPRLLIYGASTRATGIPAR | |
| | KFQGRVTITADESTSTAYMEL | | FSGSGSGTEFTLTISSLQSEDF | |
| | SSLRSEDTAVYYCAYGANGH | | AVYYCQQYSSSPLTFGQGTK | |
| | LYGMDAWGQGTTVTVSS | | VEIKR | |
| 246 | QVQLVQSGAEVKKPGSSVKV | 2668 | DIQMTQSPSSLSASVGDRVTI | 2762 |
| | SCKASGGTFTSYFMHWVRQA | | TCQATQDISNYLNWYQQKPG | |
| | PGQGLEWMGGIIPMFGAPVY | | KAPKLLIYGASNLPSGVPSRF | |
| | AQDFQGRVTITADESTSTAYM | | SGSGSGTDFTLTISSLQPEDFA | |
| | ELSSLRSEDTAVYYCAAGLDF | | TYYCQQSYSDLLTFGPGTKV | |
| | WSGPDNYYMDVWGKGTTVT | | DIKR | |
| | VSS | | | |
| 247 | QVQLVQSGAEVKKPGASVKV | 2669 | EIVMTQSPATLSVSPGERATL | 2763 |
| | SCKASGGTLMSYAISWVRQAP | | SCRGSQSISGNYLAWYQQKP | |
| | GQGLEWMGIINPRGGTTRYA | | GQAPRLLIYDTSARAAGIPAR | |
| | QKFQGRVTMTRDTSTSTVYM | | FSGSGSGTEFTLTISSLQSEDF | |
| | ELSSLRSEDTAVYYCARSEDS | | AVYYCQQYNSYPLTFGGGTK | |
| | GYDYLDYWGQGTLVTVSS | | VEIKR | |
| 248 | QVQLVQSGAEVKKPGASVKV | 2670 | DIQMTQSPSSLSASVGDRVTI | 2764 |
| | SCKASGYTFTGYYMHWVRQA | | TCRASQDLDRYLAWYQQKP | |
| | PGQGLEWMGVINPNGGSISYA | | GKAPKLLIYAASSLQTGVPSR | |
| | QKFQGRVTMTRDTSTSTVYM | | FSGSGSGTDFTLTISSLQPEDF | |
| | ELSSLRSEDTAVYYCAREGWF | | ATYYCQQYYSTPYTFGQGTK | |
| | GEDGMDVWGQGTTVTVSS | | LEIKR | |
| 249 | QVQLVQSGAEVKKPGASVKV | 2671 | EIVMTQSPATLSVSPGERATL | 2765 |
| | SCKASGYTFTSDGISWVRQAP | | SCRASQSIGNNLKAWYQQKP | |
| | GQGLEWMGWMNPNSGNAGY | | GQAPRLLIYDTSAHTTGIPAR | |
| | AQKFQGRVTMTRDTSTSTVY | | FSGSGSGTEFTLTISSLQSEDF | |
| | MELSSLRSEDTAVYYCATAV | | AVYYCQHYGNSLTFGQGTK | |
| | AGTDAFDIWGQGTMVTVSS | | VEIK | |
| 250 | QVQLVQSGAEVKKPGSSVKV | 2672 | EIVMTQSPATLSVSPGERATL | 2766 |
| | SCKASGYTLTSFAMHWVRQA | | SCRASQSVGSSSLAWYQQKP | |
| | PGQGLEWMGRIIPMSGTANY | | GQAPRLLIYGASTRATGIPAR | |
| | AQKFQGRVTITADESTSTAYM | | FSGSGSGTEFTLTISSLQSEDF | |
| | ELSSLRSEDTAVYYCASTSPD | | AVYYCQQYGSSPYTFGQGTK | |
| | QYYYGMDVWGQGTTVTVSS | | VEIKR | |
| 251 | QVQLVQSGAEVKKPGSSVKV | 2673 | EIVMTQSPATLSVSPGERATL | 2767 |
| | SCKASGGTFSSDAINWVRQAP | | SCRASQSVSSNYLAWYQQKP | |
| | GQGLEWMGGIIPIVGTPTYAQ | | GQAPRLLIYDVSTRATGIPAR | |
| | KFQGRVTITADESTSTAYMEL | | FSGSGSGTEFTLTISSLQSEDF | |
| | SSLRSEDTAVYYCAKGLAFGV | | AVYYCQQYGSSTLTFGGGTK | |
| | FDGLDVWGQGTTVTVSS | | VEIKR | |
| 252 | QVQLVQSGAEVKKPGSSVKV | 2674 | DIQMTQSPSSLSASVGDRVTI | 2768 |
| | SCKASGYTLTDLSIHWVRQAP | | TCRASQSISSYLNWYQQKPG | |
| | GQGLEWVGGIIPMSGTANYA | | KAPKLLIYAASSLQSGVPSRF | |
| | QKFQGRVTITADESTSTAYME | | SGSGSGTDFTLTISSLQPEDFA | |
| | LSSLRSEDTAVYYCARSSSSW | | TYYCQQSYSTPLTFGGGTKV | |
| | PKYFQHWGQGTLVTVSS | | EIKR | |
| 253 | QVQLVQSGAEVKKPGSSVKV | 2675 | DIQMTQSPSSLSASVGDRVTI | 2769 |
| | SCKASGYTFTTYFMHWVRQA | | TCRASQDISRWLAWYQQKPG | |
| | PGQGLEWMGGIVPVFGTTKY | | KAPKLLIYAASSLQSGVPSRF | |
| | AQKFQGRVTITADESTSTAYM | | SGSGSGTDFTLTISSLQPEDFA | |
| | ELSSLRSEDTAVYYCASSAVG | | TYYCQQYDNFPLTFGGGTKL | |
| | WFDPWGQGTLVTVSS | | EIKR | |
| 254 | QVQLVQSGAEVKKPGASVKV | 2676 | EIVMTQSPATLSVSPGERATL | 2770 |
| | SCKASGYTFTSHYMHWVRQA | | SCRASQSVSSSSLAWYQQKP | |
| | PGQGLEWMGWISPYNGNTNY | | GQAPRLLIYDTSTRATGIPAR | |
| | AQKLQGRVTMTRDTSTSTVY | | FSGSGSGTEFTLTISSLQSEDF | |
| | MELSSLRSEDTAVYYCARGES | | AVYYCQQYGTSPITFGQGTR | |
| | NSGWINFDYWGQGTLVTVSS | | LEIKR | |
| 255 | QVQLVQSGAEVKKPGSSVKV | 2677 | DIQMTQSPSSLSASVGDRVTI | 2771 |
| | SCKASGYTLTELSMHWVRQA | | TCQASHDIRNSVNWYQQKPG | |
| | PGQGLEWMGGIIPISGTVTYA | | KAPKLLIYATSSLQSGVPSRFS | |
| | QKFQGRVTITADESTSTAYME | | GSGSGTDFTLTISSLQPEDFAT | |
| | LSSLRSEDTAVYYCANKGQQL | | YYCQQSYNTPFTFGQGTKLEI | |
| | VRGYFQHWGQGTLVTVSS | | KR | |
| 256 | QVQLVQSGAEVKKPGASVKV | 2678 | DIQMTQSPSSLSASVGDRVTI | 2772 |
| | SCKASGYTFATYYLHWVRQA | | TCRASHDINNYLNWYQQKPG | |
| | PGQGLEWMGMINPSGGSTIYA | | KAPKLLIYDASNLETGVPSRF | |
| | QKFQGRVTMTRDTSTSTVYM | | SGSGSGTDFTLTISSLQPEDFA | |
| | ELSSLRSEDTAVYYCARSSGY | | TYYCQQADSFPLTFGGGTKV | |
| | DFFDYWGQGTLVTVSS | | EIKR | |
| 257 | QVQLVQSGAEVKKPGASVKV | 2679 | DIQMTQSPSSLSASVGDRVTI | 2773 |
| | SCKASGYTFTNYFMHWVRQA | | TCRASQSISSWLAWYQQKPG | |
| | PGQGLEWMGIINPSGGSTSYA | | KAPKLLIYAASTLQSGVPSRF | |
| | QKFQGRVTMTRDTSTSTVYM | | SGSGSGTDFTLTISSLQPEDFA | |
| | ELSSLRSEDTAVYYCARAHTV | | TYYCQQSYSTPWTFGQGTKL | |
| | YYYGMDVWGQGTMVTVSS | | EIKR | |
| 258 | QVQLVQSGAEVKKPGASVKV | 2680 | DIQMTQSPSSLSASVGDRVTI | 2774 |
| | SCKASGGTFGSYAISWVRQAP | | TCRASQSIKGALAWYQQKPG | |
| | GQGLEWMGWINPNTGGAHY | | KAPKLLIYSASNLQSGVPSRF | |
| | AQKFQGRVTMTRDTSTSTVY | | SGSGSGTDFTLTISSLQPEDFA | |
| | MELSSLRSEDTAVYYCARVG | | TYYCQQYNSYPLTFGGGTKV | |
| | AAAGYQHWGQGTLVTVSS | | EIKR | |
| 259 | QVQLVQSGAEVKKPGSSVKV | 2681 | DIVMTQSPDSLAVSLGERATI | 2775 |
| | SCKASGYTFTSSEINWVRQAP | | NCKSSQSLFYSSNNRNYLAW | |
| | GQGLEWMGGIHPMFGTTNYA | | YQQKPGQPPKLLIYWASTRES | |
| | QKFQGRVTITADESTSTAYME | | GVPDRFSGSGSGTDFTLTISSL | |
| | LSSLRSEDTAVYYCARARLM | | QAEDVAVYYCQQYYSIPYTF | |
| | VYAPSDYWGQGTLVTVSS | | GQGTKVEIKR | |
| 260 | QVQLVQSGAEVKKPGASVKV | 2682 | DIQMTQSPSSLSASVGDRVTI | 2776 |
| | SCKASGYTFTNYYVHWVRQA | | TCRSSQSISTYLNWYQQKPG | |
| | PGQGLEWMGMINPSGGSTNY | | KAPKLLIYGASNLQSGVPSRF | |
| | AQKFQGRVTMTRDTSTSTVY | | SGSGSGTDFTLTISSLQPEDFA | |
| | MELSSLRSEDTAVYYCARVSG | | TYYCQQVISYPITFGGGTKVE | |
| | WKRGWFDPWGQGTLVTVSS | | IKR | |
| 261 | QVQLVQSGAEVKKPGASVKV | 2683 | DIVMTQSPDSLAVSLGERATI | 2777 |
| | SCKASGYTFTRYYMHWVRQA | | NCKSSQSISHSPNTRDYLAWY | |
| | PGQGLEWMGIINPSGGSASYA | | QQKPGQPPKLLIYWASTRESG | |
| | QKFQGRVTMTRDTSTSTVYM | | VPDRFSGSGSGTDFTLTISSLQ | |
| | ELSSLRSEDTAVYYCARDLGG | | AEDVAVYYCQQYYSSPFTFG | |
| | AAAGYFDYWGQGTLVTVSS | | PGTKVDIKR | |
| 262 | QVQLVQSGAEVKKPGASVKV | 2684 | EIVMTQSPATLSVSPGERATL | 2778 |
| | SCKASGFTFSDYGYYMHWVR | | SCRASQRVGNTYLAWYQQK | |
| | QAPGQGLEWMGWMDPSSGH | | PGQAPRLLIYDVSARASGIPA | |
| | TGYAQRFQGRVTMTRDTSTST | | RFSGSGSGTEFTLTISSLQSED | |
| | VYMELSSLRSEDTAVYYCAK | | FAVYYCQQYLSPPLTFGGGT | |
| | DIGWGAFDIWGQGTTVTVSS | | KVEIKR | |
| 263 | QVQLVQSGAEVKKPGSSVKV | 2685 | EIVMTQSPATLSVSPGERATL | 2779 |
| | SCKASGGTFSSYAISWVRQAP | | SCRASQSVSSSYLAWYQQKP | |
| | GQGLEWMGGIIPIVGVANYAQ | | GQAPRLLIYDVSTRATGIPAR | |
| | KLQGRVTITADESTSTAYMEL | | FSGSGSGTEFTLTISSLQSEDF | |
| | SSLRSEDTAVYYCAKDIGGYP | | AVYYCQQYGSSPITFGQGTK | |
| | SDAFDIWGQGTTVTVSS | | VEIKR | |
| 264 | QVQLVQSGAEVKKPGSSVKV | 2686 | DIQMTQSPSSLSASVGDRVTI | 2780 |
| | SCKASGYTLTELSMHWVRQA | | TCRASQDISNYLAWYQQKPG | |
| | PGQGLEWMGGIIPISSATSIPQ | | KAPKLLIYAASSLQSGVPSRF | |
| | KFQGRVTITADESTSTAYMEL | | SGSGSGTDFTLTISSLQPEDFA | |
| | SSLRSEDTAVYYCARGALYSS | | TYYCQQYYSYPLTFGGGTKV | |
| | SPVRVVAGTKGWFDPWGQGT | | EIKR | |
| | LVTVSS | | | |
| 265 | QVQLVQSGAEVKKPGSSVKV | 2687 | EIVMTQSPATLSVSPGERATL | 2781 |
| | SCKASGHTFTSDYMHWVRQA | | SCRASQSVNSEHLAWYQQKP | |
| | PGQGLEWMGRIIPIFGTADYA | | GQAPRLLIYDTSSRATGIPARF | |
| | QKFQGRVTITADESTSTAYME | | SGSGSGTEFTLTISSLQSEDFA | |
| | LSSLRSEDTAVYYCARDDSSG | | VYYCQQYGSSPVTFGQGTKV | |
| | IFDYWGQGTLVTVSS | | EIKR | |
| 266 | QVQLVQSGAEVKKPGSSVKV | 2688 | EIVMTQSPATLSVSPGERATL | 2782 |
| | SCKASGYSLTELSIHWVRQAP | | SCRASQSVGSQLGWYQQKPG | |
| | GQGLEWMGGINPISGTANYA | | QAPRLLIYGASTRATGIPARFS | |
| | QKFQGRVTITADESTSTAYME | | GSGSGTEFTLTISSLQSEDFAV | |
| | LSSLRSEDTAVYYCARGTVRL | | YYCQQSFSTPLTFGGGTKVEI | |
| | NWFDPWGQGTLVTVSS | | KR | |
| 267 | QVQLVQSGAEVKKPGSSVKV | 2689 | DIQMTQSPSSLSASVGDRVTI | 2783 |
| | SCKASGYTLTSFGISWVRQAP | | TCRASQDISNFVAWYQQKPG | |
| | GQGLEWVGMIIPLSGTTHYAQ | | KAPKLLIYAASSLQSGVPSRF | |
| | KFQGRVTITADESTSTAYMEL | | SGSGSGTDFTLTISSLQPEDFA | |
| | SSLRSEDTAVYYCANLYGGN | | TYYCQQSFDTPYTFGQGTKL | |
| | AYYYYGMDVWGQGTTVTVS | | EIKR | |
| | S | | | |
| 268 | QVQLVQSGAEVKKPGSSVKV | 2690 | EIVMTQSPATLSVSPGERATL | 2784 |
| | SCKASGGTESTYALSWVRQAP | | SCRASQSVNNNQLAWYQQK | |
| | GQGLEWMGGVIPVFGTTDYA | | PGQAPRLLIYDTSSRATGIPAR | |
| | HKFQGRVTITADESTSTAYME | | FSGSGSGTEFTLTISSLQSEDF | |
| | LSSLRSEDTAVYYCASMIIFGA | | AVYYCQQYDTSPYTFGQGTK | |
| | GGWDAYYFQEWGQGTLVTV | | VEIKR | |
| | SS | | | |
| 269 | QVQLVQSGAEVKKPGASVKV | 2691 | EIVMTQSPATLSVSPGERATL | 2785 |
| | SCKASGGSFSSYALHWVRQAP | | SCRASQSISSSYLAWYQQKPG | |
| | GQGLEWMGLINPSGGRTSYA | | QAPRLLIYDVSARATGIPARF | |
| | QKFQGRVTMTRDTSTSTVYM | | SGSGSGTEFTLTISSLQSEDFA | |
| | ELSSLRSEDTAVYYCARDEGY | | VYYCQQYYSTPLTFGPGTKV | |
| | ATFDYWGQGTLVTVSS | | DIKR | |
| 270 | QVQLVQSGAEVKKPGASVKV | 2692 | DIQMTQSPSSLSASVGDRVTI | 2786 |
| | SCKASGGTFSSYYMHWVRQA | | TCRASQSISSYLNWYQQKPG | |
| | PGQGLEWMGWISAYNGNTNY | | KAPKLLIYDASNLETGVPSRF | |
| | AQKLQGRVTMTRDTSTSTVY | | SGSGSGTDFTLTISSLQPEDFA | |
| | MELSSLRSEDTAVYYCAKDM | | TYYCQQTYTTPLTFGQGTKV | |
| | GYYYDSSGGFDYWGQGTLVT | | EIKR | |
| | VSS | | | |
| 271 | QVQLVQSGAEVKKPGSSVKV | 2693 | EIVMTQSPATLSVSPGERATL | 2787 |
| | SCKASGGTFSSYAISWVRQAP | | SCRASQSVSYNQLAWYQQKP | |
| | GQGLEWMGGIIPIFGTANYAQ | | GQAPRLLIYDISSRAAGIPARF | |
| | KFQGRVTITADESTSTAYMEL | | SGSGSGTEFTLTISSLQSEDFA | |
| | SSLRSEDTAVYYCARDLSIGY | | VYYCQQYGGLPATFGQGTRL | |
| | YGDAFDIWGQGTMVTVSS | | EIKR | |
| 272 | QVQLVQSGAEVKKPGSSVKV | 2694 | DIQMTQSPSSLSASVGDRVTI | 2788 |
| | SCKASGYIFTNYYIQWVRQAP | | TCQASQYISNYLNWYQQKPG | |
| | GQGLEWMGGIIPIFGTVGYAQ | | KAPKLLIYDASSLESGVPSRFS | |
| | KFQGRVTITADESTSTAYMEL | | GSGSGTDFTLTISSLQPEDFAT | |
| | SSLRSEDTAVYYCARGRIGGG | | YYCQQSYSTPYTFGQGTKLEI | |
| | NDYWGQGTLVTVSS | | KR | |
| 273 | QVQLVQSGAEVKKPGSSVKV | 2695 | EIVMTQSPATLSVSPGERATL | 2789 |
| | SCKASGDTFNSYAVNWVRQA | | SCRASQSVSSSSLAWYQQKP | |
| | PGQGLEWMGGIIPSFGTPTYA | | GQAPRLLIYDASTRASGIPAR | |
| | WKFQGRVTITADESTSTAYME | | FSGSGSGTEFTLTISSLQSEDF | |
| | LSSLRSEDTAVYYCASVSYGS | | AVYYCQQYNRLPYTFGQGTK | |
| | FDYWGQGTLVTVSS | | LEIKR | |
| 274 | QVQLVQSGAEVKKPGSSVKV | 2696 | DIVMTQSPDSLAVSLGERATI | 2790 |
| | SCKASGYTFTYRYLHWVRQA | | NCKSSQSVLYSSNNKNYLAW | |
| | PGQGLEWMGRITPISGTTNYA | | YQQKPGQPPKLLIYWASTRES | |
| | QKFQGRVTITADESTSTAYME | | GVPDRFSGSGSGTDFTLTISSL | |
| | LSSLRSEDTAVYYCAKDSGQL | | QAEDVAVYYCQQYYKTPLTF | |
| | AHYGMDVWGQGTTVTVSS | | GGGTKLEIKR | |
| 275 | QVQLVQSGAEVKKPGSSVKV | 2697 | DIQMTQSPSSLSASVGDRVTI | 2791 |
| | SCKASGYTFTSYYMHWVRQA | | TCRASQSISSYLNWYQQKPG | |
| | PGQGLEWMGWMNPYSGNTG | | KAPKLLIYAASSLQSGVPSRF | |
| | YAQKFQGRVTITADESTSTAY | | SGSGSGTDFTLTISSLQPEDFA | |
| | MELSSLRSEDTAVYYCARVGS | | TYYCQQSYSTPLTFGGGTKV | |
| | GWYSDYWGQGTLVTVSS | | EIKR | |
| 276 | QVQLVQSGAEVKKPGSSVKV | 2698 | EIVMTQSPATLSVSPGERATL | 2792 |
| | SCKASGYTFTRFNIHWVRQAP | | SCRASQSVSSYLAWYQQKPG | |
| | GQGLEWMGWLNPFTGNTGY | | QAPRLLIYDTSTRATGIPARFS | |
| | ARKFQGRVTITADESTSTAYM | | GSGSGTEFTLTISSLQSEDFAV | |
| | ELSSLRSEDTAVYYCASSSSW | | YYCQQYHSSPWTFGQGTKVE | |
| | YGWFDPWGQGTLVTVSS | | IKR | |
| 277 | QVQLVQSGAEVKKPGSSVKV | 2699 | EIVMTQSPATLSVSPGERATL | 2793 |
| | SCKASGYTFTGYYMHWVRQA | | SCRASQSVDNLVGWYQQKP | |
| | PGQGLEWMGWIDPNSGGTNY | | GQAPRLLIYDISSRATGIPARF | |
| | AQKFQGRVTITADESTSTAYM | | SGSGSGTEFTLTISSLQSEDFA | |
| | ELSSLRSEDTAVYYCARDVDT | | VYYCQQYGRSPITFGQGTRLE | |
| | AMVTDYWGRGTLVTVSS | | IKR | |
| 278 | QVQLVQSGAEVKKPGASVKV | 2700 | DIVMTQSPDSLAVSLGERATI | 2794 |
| | SCKASGYTFTSYYMHWVRQA | | NCKSSQSVLYSSNNENYLAW | |
| | PGQGLEWMGIINPSSGSTTYA | | YQQKPGQPPKLLIYWASTRES | |
| | QKFQGRVTMTRDTSTSTVYM | | GVPDRFSGSGSGTDFTLTISSL | |
| | ELSSLRSEDTAVYYCARSVGA | | QAEDVAVYYCQQYYSLPVTF | |
| | TSAFDIWGQGTMVTVSS | | GQGTKLEIKR | |
| 279 | QVQLVQSGAEVKKPGASVKV | 2701 | DIQMTQSPSSLSASVGDRVTI | 2795 |
| | SCKASGYTFTKYYMHWVRQA | | TCRASQSISSSLNWYQQKPGK | |
| | PGQGLEWMGIINPSGGSTSYA | | APKLLIYKASSLESGVPSRFSG | |
| | QKFQGRVTMTRDTSTSTVYM | | SGSGTDFTLTISSLQPEDFATY | |
| | ELSSLRSEDTAVYYCARGRGY | | YCQQYYSYPPTFGGGTKVEI | |
| | SYGYLDYWGQGTLVTVSS | | KR | |
| 280 | QVQLVQSGAEVKKPGASVKV | 2702 | DIQMTQSPSSLSASVGDRVTI | 2796 |
| | SCKASGYTFTRYYMHWVRQA | | TCQASQDISNYLNWYQQKPG | |
| | PGQGLEWMGIINPSGGSTSYA | | KAPKLLIYQASNKDTGVPSRF | |
| | QKFQGRVTMTRDTSTSTVYM | | SGSGSGTDFTLTISSLQPEDFA | |
| | ELSSLRSEDTAVYYCARGETR | | TYYCQQSYSTPPTFGQGTKLE | |
| | SYAPYGMDVWGQGTTVTVSS | | IKR | |
| 28 | QVQLVQSGAEVKKPGASVKV | 2703 | EIVMTQSPATLSVSPGERATL | 2797 |
| | SCKASGYTFNSYGISWVRQAP | | SCRASQSVSSSYLAWYQQKP | |
| | GQGLEWMGIINPTGGSTTYAQ | | GQAPRLLIYDASARAAGIPAR | |
| | KFQGRVTMTRDTSTSTVYME | | FSGSGSGTEFTLTISSLQSEDF | |
| | LSSLRSEDTAVYYCAKDPFVM | | AVYYCQQYYSTPYTFGQGTK | |
| | DVWGQGTTVTVSS | | LEIKR | |
| 282 | QVQLVQSGAEVKKPGASVKV | 2704 | EIVMTQSPATLSVSPGERATL | 2798 |
| | SCKASGGTFSSYAISWVRQAP | | SCRASRSISDYLAWYQQKPG | |
| | GQGLEWMGWMNPNSGDTGY | | QAPRLLIYDASSRATGIPARFS | |
| | AQKFQGRVTMTRDTSTSTVY | | GSGSGTEFTLTISSLQSEDFAV | |
| | MELSSLRSEDTAVYYCARDFE | | YYCQQYYTTPLTFGQGTKVE | |
| | GGGWFDPWGQGTLVTVSS | | IKR | |
| 283 | QVQLVQSGAEVKKPGASVKV | 2705 | DIQMTQSPSSLSASVGDRVTI | 2799 |
| | SCKASGYTFTSYYMDWVRQA | | TCRASQSISSYLNWYQQKPG | |
| | PGQGLEWMGRINPSSGSTTYV | | KAPKLLIYAASSLQSGVPSRF | |
| | QKFQGRVTMTRDTSTSTVYM | | SGSGSGTDFTLTISSLQPEDFA | |
| | ELSSLRSEDTAVYYCARTPSG | | TYYCQQSYSTPWTFGQGTKV | |
| | SYSDFDYWGQGTLVTVSS | | EIKR | |
| 284 | QVQLVQSGAEVKKPGASVKV | 2706 | DIQMTQSPSSLSASVGDRVTI | 2800 |
| | SCKASGYTFTSYYMHWVRQA | | TCRASHNISTWLAWYQQKPG | |
| | PGQGLEWMGVINPSGGSTTY | | KAPKLLIYAASSLQSGVPSRF | |
| | AQKFQGRVTMTRDTSTSTVY | | SGSGSGTDFTLTISSLQPEDFA | |
| | MELSSLRSEDTAVYYCARVPG | | TYYCQQSYSTPPTFGQGTRLE | |
| | VSPGDYGMDVWGQGTTVTVS | | IKR | |
| | S | | | |
| 285 | QVQLVQSGAEVKKPGSSVKV | 2707 | DIVMTQSPDSLAVSLGERATI | 2801 |
| | SCKASGYSFTNYYMHWVRQA | | NCKSSQSVLYSSNNKNYLAW | |
| | PGQGLEWMGGIIPVFGTTTYS | | YQQKPGQPPKLLIYWASTRES | |
| | QTFQGRVTITADESTSTAYME | | GVPDRFSGSGSGTDFTLTISSL | |
| | LSSLRSEDTAVYYCARESQDG | | QAEDVAVYYCQQYYSSPLTF | |
| | DFDYWGQGTLVTVSS | | GGGTKVEIKR | |
| 286 | QVQLVQSGAEVKKPGASVKV | 2708 | EIVMTQSPATLSVSPGERATL | 2802 |
| | SCKASGYTFTSYGISWVRQAP | | SCRASQSVSSSYLAWYQQKP | |
| | GQGLEWMGWISPNSGVTNYA | | GQAPRLLIYDVSTRASGIPAR | |
| | QKFQGRVTMTRDTSTSTVYM | | FSGSGSGTEFTLTISSLQSEDF | |
| | ELSSLRSEDTAVYYCVSDDYG | | AVYYCQQYNNWPYTFGQGT | |
| | AFDYWGQGTLVTVSS | | KLEIKR | |
| 287 | QVQLVQSGAEVKKPGASVKV | 2709 | DIQMTQSPSSLSASVGDRVTI | 2803 |
| | SCKASGYTFTRHYVHWVRQA | | TCRASQSISSSLAWYQQKPGK | |
| | PGQGLEWVGIINPSSGSASYA | | APKLLIYAASSLQSGVPSRFS | |
| | QKFQGRVTMTRDTSTSTVYM | | GSGSGTDFTLTISSLQPEDFAT | |
| | ELSSLRSEDTAVYYCARDRLR | | YYCQQSYTIPPTFGQGTKLEI | |
| | SRFDYWGQGTLVTVSS | | KR | |
| 288 | QVQLVQSGAEVKKPGASVKV | 2710 | DIQMTQSPSSLSASVGDRVTI | 2804 |
| | SCKASGYTFTTYDINWVRQAP | | TCRASQGISNNLNWYQQKPG | |
| | GQGLEWMGWMNPSSGNSGF | | KAPKLLIYKASTLESGVPSRF | |
| | AQQFQGRVTMTRDTSTSTVY | | SGSGSGTDFTLTISSLQPEDFA | |
| | MELSSLRSEDTAVYYCAREDY | | TYYCQQSYSTPITFGQGTKVE | |
| | YDSSGYYNWGQGTLVTVSS | | IKR | |
| 289 | QVQLVQSGAEVKKPGASVKV | 2711 | DIQMTQSPSSLSASVGDRVTI | 2805 |
| | SCKASGYTFTSYGISWVRQAP | | TCQASQGITSYLNWYQQKPG | |
| | GQGLEWMGWMNPISGNTDY | | KAPKLLIYKASSLESGVPSRFS | |
| | APNFQGRVTMTRDTSTSTVY | | GSGSGTDFTLTISSLQPEDFAT | |
| | MELSSLRSEDTAVYYCVVERR | | YYCQQGYSTPLTFGGGTKVEI | |
| | REVGMDVWGQGTTVTVSS | | KR | |
| 290 | QVQLVQSGAEVKKPGASVKV | 2712 | DIQMTQSPSSLSASVGDRVTI | 2806 |
| | SCKASGGTFTSYYMHWVRQA | | TCRASQSISSYLNWYQQKPG | |
| | PGQGLEWMGWISAYNGKTDY | | KAPKLLIYDASNLETGVPSRF | |
| | AQKFQGRVTMTRDTSTSTVY | | SGSGSGTDFTLTISSLQPEDFA | |
| | MELSSLRSEDTAVYYCARDQ | | TYYCQQTYSAPPTFGQGTKL | |
| | GYYYDSSGAFDIWGQGTLVT | | EIKR | |
| | VSS | | | |
| 291 | QVQLVQSGAEVKKPGASVKV | 2713 | DIVMTQSPLSLPVTPGEPASIS | 2807 |
| | SCKASGYTFTSYYMHWVRQA | | CRSSQSLLHSNGYNYLDWYL | |
| | PGQGLEWMGIINPSGGSTVYA | | QKPGQSPQLLIYLGSNRASGV | |
| | QTFQGRVTMTRDTSTSTVYM | | PDRFSGSGSGTDFTLKISRVE | |
| | ELSSLRSEDTAVYYCARGIGS | | AEDVGVYYCMQGLQTPYTF | |
| | KGAFDIWGQGTMVTVSS | | GQGTRLEIKR | |
| 292 | QVQLVQSGAEVKKPGASVKV | 2714 | DIQMTQSPSSLSASVGDRVTI | 2808 |
| | SCKASGYTFTSYGISWVRQAP | | TCRASQSISTYVNWYQQKPG | |
| | GQGLEWMGWINPNSGGTNYA | | KAPKLLIYDTSSLQSGVPSRFS | |
| | QKFQGRVTMTRDTSTSTVYM | | GSGSGTDFTLTISSLQPEDFAT | |
| | ELSSLRSEDTAVYYCARQGGL | | YYCQQSFITPPTFGQGTKLEIK | |
| | RDFDYWGQGTLVTVSS | | R | |
| 293 | QVQLVQSGAEVKKPGSSVKV | 2715 | DIVMTQSPLSLPVTPGEPASIS | 2809 |
| | SCKASGYMFTTPYIHWVRQAP | | CRSSQSLLHSNGYNYLDWYL | |
| | GQGLEWMGVINPISGTTTYAQ | | QKPGQSPQLLIYLGSNRASGV | |
| | KFQGRVTITADESTSTAYMEL | | PDRFSGSGSGTDFTLKISRVE | |
| | SSLRSEDTAVYYCANDRHYDF | | AEDVGVYYCMQALQTPTFG | |
| | WSGYYKEEWEYFQHWGQGT | | GGTKVEIKR | |
| | LVTVSS | | | |
| 294 | QVQLVQSGAEVKKPGASVKV | 2716 | DIQMTQSPSSLSASVGDRVTI | 2810 |
| | SCKASGYTFTSNNMHWVRQA | | TCRASQGIRNDLGWYQQKPG | |
| | PGQGLEWMGWINLNSGGTNY | | KAPKLLIYQASSLENGVPSRF | |
| | AQKFQGRVTMTRDTSTSTVY | | SGSGSGTDFTLTISSLQPEDFA | |
| | MELSSLRSEDTAVYYCAKAID | | TYYCQQAYSLPWTFGQGTKL | |
| | YYYMDVWGKGTTVTVSS | | EIKR | |

The polypeptides above were tested as disclosed above in Examples 4 and 5. Data is disclosed below in Table 18c, reporting FACS fold change over parental as (−), indicating <2 fold; (+), indicating 2-10 fold; (++), indicating 10-30 fold; and (+++), indicating >30 fold.

TABLE 18c
Polypeptide Activity (FACS & BLI)

| | FLT3 Mutant | FLT3 WT | FLT3 | |
|---|---|---|---|---|
| | Geometric Mean | Geometric Mean | Mutant BLI/Octet | FLT3 WT BLI/Octet |
| Polypeptide | Fold Change over | Fold Change over | Binding Summary | Binding Summary |
| No. | Jurkat Parental | Jurkat Parental | (Yes/No/Ambiguous) | (Yes/No/Ambiguous) |
| 201 | − | − | Yes | Yes |
| 202 | + | − | Yes | No |
| 203 | − | − | Yes | No |
| 204 | − | − | Ambiguous | Ambiguous |
| 205 | − | − | Yes | Yes |
| 206 | − | − | Yes | Yes |
| 207 | ++ | ++ | Yes | Yes |
| 208 | ++ | ++ | Yes | Yes |
| 209 | − | − | Yes | Yes |
| 210 | + | − | Yes | Yes |
| 211 | − | − | Ambiguous | Ambiguous |
| 212 | ++ | ++ | Yes | Yes |
| 213 | ++ | ++ | Yes | Yes |
| 214 | +++ | +++ | Yes | Yes |
| 215 | ++ | ++ | Yes | Yes |
| 216 | − | − | Yes | Yes |
| 217 | − | − | Yes | Yes |
| 218 | ++ | ++ | Yes | Yes |
| 219 | ++ | ++ | Yes | Yes |
| 220 | − | − | Yes | No |
| 221 | + | − | Yes | No |
| 222 | +++ | +++ | Yes | Yes |
| 223 | +++ | +++ | Yes | Yes |
| 224 | + | − | Yes | Yes |
| 225 | + | − | Yes | Yes |
| 226 | + | − | Yes | Yes |
| 227 | − | − | Yes | Yes |
| 228 | +++ | +++ | Yes | Yes |
| 229 | +++ | +++ | Yes | Yes |
| 230 | − | − | Yes | Yes |
| 231 | +++ | +++ | Yes | Yes |
| 232 | − | − | Yes | Yes |
| 233 | +++ | +++ | Yes | Yes |
| 234 | − | − | Yes | Yes |
| 235 | +++ | +++ | Yes | Yes |
| 236 | − | − | Yes | Yes |
| 237 | +++ | +++ | Yes | Yes |
| 238 | +++ | +++ | Yes | Yes |
| 239 | − | − | Yes | Yes |
| 240 | +++ | +++ | Yes | Yes |
| 241 | ++ | ++ | Yes | Yes |
| 242 | +++ | +++ | Yes | Yes |
| 243 | + | + | Yes | Yes |
| 244 | +++ | +++ | Yes | Yes |
| 245 | + | − | Yes | Yes |
| 246 | + | ++ | Yes | Yes |
| 247 | + | + | Yes | Yes |
| 248 | + | + | Yes | Yes |
| 249 | + | + | Yes | Yes |
| 250 | + | − | Yes | Yes |
| 251 | − | − | Yes | Yes |
| 252 | − | − | Yes | Yes |
| 253 | + | + | Yes | Yes |
| 254 | + | ++ | Yes | Yes |
| 255 | − | − | Yes | Yes |
| 256 | +++ | +++ | Yes | Yes |
| 257 | ++ | ++ | Yes | Yes |
| 258 | − | − | Ambiguous | Ambiguous |
| 259 | ++ | ++ | Yes | Yes |
| 260 | +++ | +++ | Yes | Yes |
| 261 | +++ | +++ | Yes | Yes |
| 262 | ++ | ++ | Yes | Yes |
| 263 | + | − | Yes | Yes |
| 264 | − | − | Yes | Yes |
| 265 | ++ | ++ | Yes | Yes |
| 266 | − | − | Yes | Yes |
| 267 | − | − | Yes | Yes |
| 268 | + | − | Yes | Yes |
| 269 | ++ | ++ | Yes | Yes |
| 270 | ++ | ++ | Yes | Yes |
| 271 | − | − | Yes | Yes |
| 272 | − | − | Yes | Yes |
| 273 | ++ | ++ | Yes | Yes |
| 274 | − | − | Yes | Yes |
| 275 | − | − | Yes | Yes |
| 276 | + | − | Yes | Yes |
| 277 | − | − | Yes | Yes |
| 278 | +++ | +++ | Yes | Yes |
| 279 | +++ | +++ | Yes | Yes |
| 280 | +++ | +++ | Yes | Yes |
| 281 | + | − | Yes | Yes |
| 282 | + | − | Yes | Yes |
| 283 | +++ | +++ | Yes | Yes |
| 284 | +++ | +++ | Yes | Yes |
| 285 | − | − | Yes | Yes |
| 286 | + | − | Yes | Yes |
| 287 | + | ++ | Yes | Yes |
| 288 | ++ | + | Yes | Yes |
| 289 | − | − | Yes | Yes |
| 290 | ++ | ++ | Yes | Yes |
| 291 | +++ | +++ | Yes | Yes |
| 292 | + | + | Yes | Yes |
| 293 | + | + | Yes | Yes |
| 294 | ++ | ++ | Yes | Yes |

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

1. A polypeptide which selectively binds a first polymorphic variant of a human cancer cell antigen over a second polymorphic variant of the human cancer cell antigen; or selectively binds the second polymorphic variant of the antigen over the first polymorphic variant of the antigen.

2. The polypeptide of claim 1, wherein the antigen is chosen from CD33, CLL-1, and FLT3.

3. (canceled)

4. A polypeptide which selectively binds a first polymorphic variant of CD33 over a second polymorphic variant of CD33; or selectively binds the second polymorphic variant of CD33 over the first polymorphic variant of CD33; wherein the binding is at least 2-fold selective.

5. (canceled)

6. (canceled)

7. The polypeptide of claim 4, wherein the first polymorphic variant of CD33 is R69 and the second polymorphic variant of CD33 is G69; or the first polymorphic variant of CD33 is G69 and the second polymorphic variant of CD33 is R69.

8. (canceled)

9. (canceled)

10. The polypeptide of claim 7, comprising three heavy chain variable (V_{H}) domain CDRs HCDR1, HCDR2, and HCDR3; wherein:
HCDR1 comprises an amino acid sequence with at least 95% sequence identity to an amino acid sequence chosen from SEQ ID NOs:1-25 and 201-217,
HCDR2 comprises an amino acid sequence with at least 95% sequence identity to an amino acid sequence chosen from SEQ ID NOs:26-50 and 218-234,
HCDR3 comprises an amino acid sequence with at least 95% sequence identity to an amino acid sequence chosen from SEQ ID NOs:51-75 and 235-251.

11. (canceled)

12. (canceled)

13. (canceled)

14. (canceled)

15. The polypeptide of claim 7, comprising three light chain variable (V_{L}) domain CDRs LCDR1, LCDR2, and LCDR3, wherein:
LCDR1 comprises an amino acid sequence with at least 95% sequence identity to an amino acid sequence chosen from SEQ ID NOs:76-100 and 252-268,
LCDR2 comprises an amino acid sequence with at least 95% sequence identity to an amino acid sequence chosen from SEQ ID NOs:101-125 and 269-285, and
LCDR3 comprises an amino acid sequence with at least 95% sequence identity to an amino acid sequence chosen from SEQ ID NOs:126-150 and 286-302.

16. (canceled)

17. (canceled)

18. (canceled)

19. (canceled)

20. The polypeptide of claim 7, comprising a V_{H} domain having an amino acid sequence exhibiting at least 95% sequence identity to a sequence chosen from any of SEQ ID NOs 151-175 and 303-319.

21. (canceled)

22. (canceled)

23. (canceled)

24. (canceled)

25. The polypeptide of claim 7, comprising a V_{L} domain having an amino acid sequence exhibiting at least 95% sequence identity to a sequence chosen from any of SEQ ID NOs 176-200 and 320-336.

26. (canceled)

27. (canceled)

28. (canceled)

29. (canceled)

30. (canceled)

31. (canceled)

32. (canceled)

33. (canceled)

34. (canceled)

35. A polypeptide which selectively binds a first polymorphic variant of FLT3 over a second polymorphic variant of FLT3; or selectively binds the second polymorphic variant of FLT3 over the first polymorphic variant; wherein the binding is at least 2-fold selective.

36. (canceled)

37. (canceled)

38. The polypeptide of claim 35, wherein the first polymorphic variant of FLT3 is T227 and the second polymorphic variant of FLT3 is M227; or first polymorphic variant of FLT3 is M227 and the second polymorphic variant of FLT3 is T227.

39. (canceled)

40. A polypeptide which selectively binds a first polymorphic variant of CLL-1 over a second polymorphic variant of CLL-1; or selectively binds the second polymorphic variant of CLL-1 over the first polymorphic variant; wherein the binding is at least 2-fold selective.

41. (canceled)

42. (canceled)

43. The polypeptide of claim 40, wherein the first polymorphic variant of CLL-1 is K224 and the second polymorphic variant of CLL-1 is Q244; or first polymorphic variant of CLL-1 is Q224 and the second polymorphic variant of CLL-1 is K244.

44. (canceled)

45. (canceled)

46. The polypeptide of claim 43, comprising three heavy chain variable (V_{H}) domain CDRs HCDR1, HCDR2, and HCDR3, wherein:
HCDR1 comprises an amino acid sequence with at least 95% sequence identity to an amino acid sequence chosen from SEQ ID NOs:337-360- and 529-550,
HCDR2 comprises an amino acid sequence with at least 95% sequence identity to an amino acid sequence chosen from SEQ ID NOs:361-384 and 551-572, and
HCDR3 comprises an amino acid sequence with at least 95% sequence identity to an amino acid sequence chosen from SEQ ID NOs:385-408 and 573-594.

47. (canceled)

48. (canceled)

49. (canceled)

50. (canceled)

51. The polypeptide of claim 43, comprising three light chain variable (V_{L}) domain CDRs LCDR1, LCDR2, and LCDR3, wherein:
LCDR1 comprises an amino acid sequence with at least 95% sequence identity to an amino acid sequence chosen from SEQ ID NOs:409-432 and 595-616,
LCDR2 comprises an amino acid sequence with at least 95% sequence identity to an amino acid sequence chosen from SEQ ID NOs:433-456 and 617-638, and
LCDR3 comprises an amino acid sequence with at least 95% sequence identity to an amino acid sequence chosen from SEQ ID NOs:457-480 and 639-660.

52. (canceled)

53. (canceled)

54. (canceled)

55. (canceled)

56. The polypeptide of claim 43, comprising a V_{H} domain having an amino acid sequence exhibiting at least 95% sequence identity to a sequence chosen from any of SEQ ID NOs 151-175 and 303-319.

57. (canceled)

58. (canceled)

59. (canceled)

60. (canceled)

61. The polypeptide of claim 43, comprising a V_{L} domain having an amino acid sequence exhibiting at least 95% sequence identity to a sequence chosen from any of SEQ ID NOs 176-200 and 320-336.

62.-69. (canceled)

70. A single-chain variable fragment (scFv) comprising the polypeptide of claim 1.

71. A monoclonal antibody (mAb), or an antigen-binding fragment thereof, comprising the polypeptide of claim 1.

72.-77. (canceled)

78. An antibody-drug conjugate (ADC) comprising the mAb, or antigen-binding fragment thereof, of claim 71.

79. (canceled)

80. (canceled)

81. A chimeric antigen receptor (CAR) comprising an extracellular ligand binding domain comprising a polypeptide of claim 1.

82.-94. (canceled)

95. A nucleotide sequence encoding any of the polypeptides, scFvs, mAbs, or CARs of claim 1.

96. A vector comprising the nucleotide sequence of claim 95.

97. (canceled)

98. (canceled)

99. An engineered immune effector cell expressing at the cell surface a CAR of claim 81.

100.-113. (canceled)

114. A method of treatment of a subject in need thereof, who has a first polymorphic variant of an antigen on the surface of a target cell, comprising:
a. optionally, treating the subject with one or more conditioning regimens to deplete the subject of target cells bearing the first polymorphic variant of the antigen;
b. administering to the subject either:
a population of engineered immune effector cells that express a chimeric antigen receptor (CAR) that selectively binds the first polymorphic variant of the antigen on the surface of the target cell; or
a monoclonal antibody (mAb), or antigen-binding fragment thereof, that selectively binds the first polymorphic variant of the antigen on the surface of the target cell; or
an antibody-drug conjugate (ADC) comprising monoclonal antibody (mAb), or antigen-binding fragment thereof, that selectively binds the first polymorphic variant of the antigen on the surface of the target cell; and
c. administering to the subject a population of donor hematopoietic cells, a plurality of which comprise a second polymorphic variant of the antigen;
wherein the administering of the hematopoietic cells, and the administering of the CAR-expressing cells, mAb, or ADC, may be done concurrently, or sequentially in either order.

115. A method of immunotherapy of a human subject in need thereof, who has a first polymorphic variant of an antigen on the surface of a target cell, comprising:
a. optionally, treating the subject with one or more conditioning regimens to deplete the subject of target cells bearing the first polymorphic variant of the antigen;
b. administering to the subject a population of donor hematopoietic cells, a plurality of which comprise a second polymorphic variant of the antigen; and
c. administering to the subject:
a population of engineered immune effector cells that express a chimeric antigen receptor (CAR) that specifically binds the first polymorphic variant of an antigen on the surface of a target cell; or
a monoclonal antibody (mAb) or antigen-binding fragment thereof, that selectively binds the first polymorphic variant of the antigen on the surface of the target cell; or
an antibody-drug conjugate (ADC) comprising a monoclonal antibody (mAb), or antigen-binding fragment thereof, that selectively binds the first polymorphic variant of the antigen on the surface of the target cell.

116. A method of treatment of a subject in need thereof, who has a first polymorphic variant of an antigen on the surface of a target cell, comprising:
a. administering to the subject:
a population of engineered immune effector cells that express a chimeric antigen receptor (CAR) which binds the antigen on the surface of the target cell; or
a monoclonal antibody (mAb) which binds the antigen on the surface of the target cell; or
an antibody-drug conjugate (ADC) comprising a monoclonal antibody (mAb) which binds the antigen on the surface of the target cell; and
b. optionally, treating the subject with one or more conditioning regimens to deplete the subject of target cells bearing the first polymorphic variant of the antigen;
c. administering to the subject either:
a population of engineered immune effector cells that express a chimeric antigen receptor (CAR) that selectively binds the first polymorphic variant of the antigen on the surface of the target cell; or
a monoclonal antibody (mAb), or antigen-binding fragment thereof, that selectively binds the first polymorphic variant of the antigen on the surface of the target cell; or
an antibody-drug conjugate (ADC) comprising a monoclonal antibody (mAb), or antigen-binding fragment thereof, that selectively binds the first polymorphic variant of the antigen on the surface of the target cell; and
d. administering to the subject a population of donor hematopoietic cells, a plurality of which comprise a second polymorphic variant of the antigen;
wherein the administering of the hematopoietic cells, and the administering of the CAR-expressing cells, mAb, or ADC, may be done concurrently, or sequentially in either order.

117.-153. (canceled)
